(19) United States
(12) Patent Application Publication
(10) Pub. No.: US 2014/0057895 A1
(43) Pub. Date: Feb. 27, 2014

(54) INDAZOLE- AND PYRROLOPYRIDINE-DERIVATIVE AND PHARMACEUTICAL USE THEREOF

(71) Applicants: Kazuhiro Mizuno, Osaka-shi (JP); Junya Ikeda, Osaka-shi (JP); Takanori Nakamura, Osaka-shi (JP); Masato Iwata, Osaka-shi (JP); Hiromichi Otaka, Suita-shi (JP); Nana Goto, Suita-shi (JP)

(72) Inventors: Kazuhiro Mizuno, Osaka-shi (JP); Junya Ikeda, Osaka-shi (JP); Takanori Nakamura, Osaka-shi (JP); Masato Iwata, Osaka-shi (JP); Hiromichi Otaka, Suita-shi (JP); Nana Goto, Suita-shi (JP)

(21) Appl. No.: 14/005,659

(22) PCT Filed: Jun. 6, 2012
(86) PCT No.: PCT/JP2012/065052

§ 371 Date: Oct. 16, 2013

(30) Foreign Application Priority Data
Jun. 7, 2011 (JP) 2011-127688

(51) Int. Cl. C07D 471/04; C07D 413/04; C07D 417/14; C07D 413/14

(52) U.S. Cl. CPC C07D 471/04; C07D 413/14; C07D 413/04; C07D 417/14
USPC 514/210.21; 546/199; 514/322; 546/187; 514/316; 544/138; 514/234.5; 548/133; 514/364; 546/113; 514/300

(57) ABSTRACT

The present invention relates to a novel indazole- or pyrrolopyridine-derivative comprising a 5 membered heterocyclic substituent at 1 position thereof which has an agonistic action or a partial agonistic action against serotonin-4 receptor, and a pharmaceutical composition comprising the same.

### TECHNICAL FIELD

The present invention relates to a novel indazole- or pyrrolopyridine-derivative which has an agonistic action or a partial agonistic action against serotonin-4 receptor (hereinafter, optionally referred to as 5-HT₄ receptor), and a pharmaceutical composition comprising the same.

### BACKGROUND ART

5-HT₄ receptor which is a subtype of serotonin receptor has been found in an action mechanism study of metoclopramide [i.e. 4-amino-5-chloro-N-(2-diethylamino-ethyl)-2-methoxybenzamide] which is an enterokinesis-promoting agent or a digestive tract function-improving agent in widespread clinical use (see, Non-patent Reference 1). It has been known that 5-HT₄ receptor agonists promote enterokinesis in the peripheral part, and for example, mosapride, cisapride and tegaserod have already been marketed (provided that the sale of cisapride was stopped after marketing). On the other hand, it has been reported that in the central nerve system, 5-HT₄ receptor agonists are effective in improving cognitive function by enhancing the acetylcholine release, and in increasing soluble APP α via the activation of α secretase to lower the amount of beta-amyloid protein (Aβ) relatively (see, Non-patent Reference 2). PRX-03140 which acts as a partial agonist to 5-HT₄ receptor has been reported to be efficacious for improving cognitive function and lowering Aβ in an animal experiment using rats (see, Non-patent Reference 1). Furthermore, it has been reported that PRX-03140 shows the effect for improving cognitive function in a phase II clinical trial with AD patients (see, Non-patent Reference 2). Thus, 5-HT₄ receptor agonists are expected to be a medicament having a novel mechanism for treating various dementia caused by Alzheimer-type dementia (AD) and neurodegenerative diseases. Meanwhile, a super-aging society is coming in the near future, and the number of patients suffering from Alzheimer-type dementia (AD) is increasing rapidly. Hence, it has been strongly desired to develop an efficacious medicament for treating Alzheimer-type dementia.

It has also been known that an amide derivative having an indazole is useful as an enterokinesis-promoting agent or a digestive tract function-improving agent (see, Patent References 1 and 2).

However, there are no reports on an indazole or pyrrolopyridine compound wherein the nitrogen atom at 1-position of the indazole or pyrrolopyridine ring binds to an oxadiazole ring and the like.

### PRIOR ART DOCUMENT

### Patent Reference

Patent REFERENCE 1: US 2005/197335 A1
Patent Reference 2: US 2006/135764 A1

### Non-Patent Reference

Non-patent Reference 1: 37th SFN Meeting (2007), presentation abstract (poster presentation number 745.10/CCC12)
Non-patent Reference 2: International Conference on Alzheimer's Disease (ICAD) 2008, presentation abstract, poster presentation number HT-01-07

### DISCLOSURE OF INVENTION

### Problems to Be Solved by Invention

The problem to be solved by the present invention is to provide a serotonin-4 receptor agonist useful as a medicament for treating Alzheimer-type dementia and other similar diseases.

### Means of Solving Problems

The present inventors have extensively studied the problem and have found that a group of compounds comprising an aromatic-ring moiety of indazole or pyrrolopyridine and a bioisosteric structure of amide bond as a linker moiety to bind the aromatic-ring moiety and an amine side chain (typically, oxadiazole ring) shows an excellent agonistic activity against 5-HT₄ receptors, and thus useful as a medicament for treating Alzheimer-type dementia and similar diseases. Based upon the new findings, the present invention has been completed. The present invention can provide indazole derivatives and pyrrolopyridine derivatives of the following Formula (1) (hereinafter, optionally referred to as “the present compound”).

### Term 1

A compound of Formula (1):

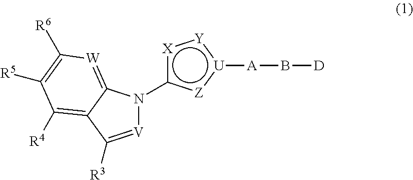

or a pharmaceutically acceptable salt thereof wherein
A is the following Formula (A-1), Formula (A-2), Formula (A-3), or Formula (A-4):

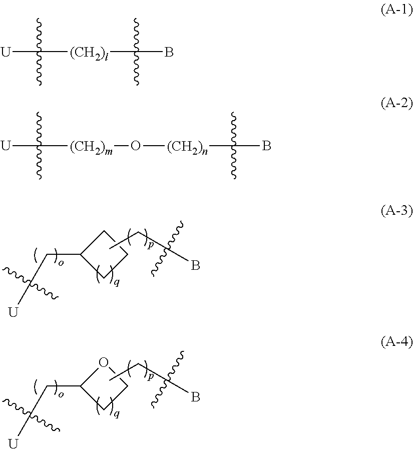

wherein
l is an integer of 0 to 4,
m is an integer of 0 to 2,
n is an integer of 0 to 2,
o and p are independently an integer of 0 or 1,
q is an integer of 0 to 5,
(A-1) to (A-4) may be independently and optionally substituted with one or more substituents independently-selected from the group consisting of C₁₋₆ alkyl group, C₂₋₆ alkenyl group, C₂₋₆ alkynyl group, hydroxy group, C₁₋₆ alkoxy group, and halogen atom at each substitutable position thereof,
B is the following Formula (B-1), Formula (B-2), or Formula (B-3):

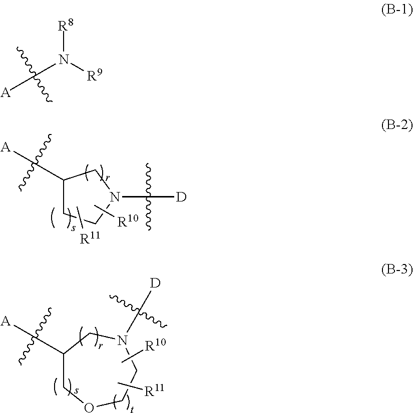

wherein (B-2) and (B-3) may optionally include an unsaturated bond(s) at an acceptable position(s) of the ring,
R⁸, R⁹ and D are independently a group selected from the group consisting of the following (1) and (2):
(1) hydrogen atom, an optionally-substituted C₁₋₆ alkyl group, an optionally-substituted C₃₋₆ alkenyl group, an optionally-substituted C₃₋₆ alkynyl group, an optionally-substituted C₃₋₆ monocyclic, C₇₋₇₀ bicyclic or C₇₋₁₂ tricyclic cycloalkyl group, and an optionally-substituted C₅₋₆ monocyclic or C₇₋₁₀ bicyclic cycloalkenyl group

wherein the C₁₋₆ alkyl group, C₃₋₆ alkenyl group, C₃₋₆ alkynyl group, C₃₋₆ monocyclic, C₇₋₁₀ bicyclic or C₇₋₁₂ tricyclic cycloalkyl group, and C₅₋₆ monocyclic or C₇₋₇₀ bicyclic cycloalkenyl group may be independently and optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, aryl group, heteroaryl group, aryloxy group, C₂₋₆ alkanoyl group, phenacyl group, and halogen atom at each substitutable position thereof;

(2) —(CH₂)ᵤ—R¹²

wherein u is an integer of 0 to 4 provided that when u is an integer of 1 to 4, the alkylene chain may be optionally substituted with one or more substituents independently-selected from the group consisting of C₁₋₆ alkyl group, C₂₋₆ alkenyl group, C₂₋₆ alkynyl group, hydroxy group, C₁₋₆ alkoxy group, oxo group, and halogen atom,

R¹² is the following Formula (R¹²-1), Formula (R¹²-2), Formula (R¹²-3), Formula (R¹²-4), Formula (R¹²-5), Formula (R¹²-6) Formula (R¹²-7), or Formula (R¹²-8):

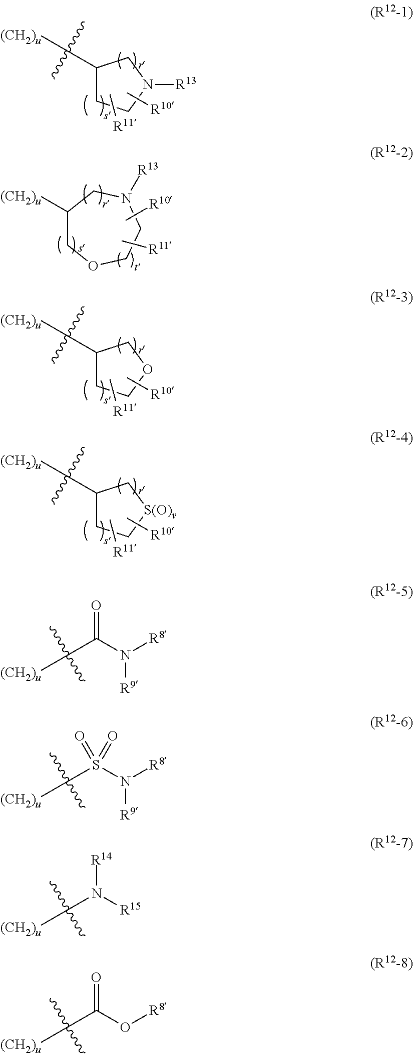

wherein R¹³ is a group selected from the group consisting of the following (1) to (5):
(1) hydrogen atom and formyl group;
(2) an optionally-substituted C₁₋₆ alkyl group, an optionally-substituted C₃₋₆ alkenyl group, an optionally-substituted C₃₋₆ alkynyl group, an optionally-substituted C₃₋₈ cycloalkyl group, and an optionally-substituted C₅₋₈ cycloalkenyl group

wherein the C₁₋₆ alkyl group, C₃₋₆ alkenyl group, C₃₋₆ alkynyl group, C₃₋₈ cycloalkyl group, and C₅₋₈ cycloalkenyl group may be independently and optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, and halogen atom at each substitutable position thereof;
(3) —COR¹⁶, —CSR¹⁶, —SO₂R¹⁶, —CO—COR¹⁶, —COOR¹⁶, and —CO—COOR¹⁶

wherein R¹⁶ is an optionally-substituted C₁₋₆ alkyl group, an optionally-substituted C₃₋₆ alkenyl group, an optionally-substituted C₃₋₆ alkynyl group, an optionally-substituted C₃₋₈ cycloalkyl group, an optionally-substituted C₅₋₈ cycloalkenyl group, an optionally-substituted aryl group, an optionally-substituted heteroaryl group, an optionally-substituted 5- to 9-membered monocyclic or 7- to 10-membered bicyclic non-aromatic unsaturated heterocyclic group (wherein the binding site is any one carbon atom in the heterocyclic ring), or an optionally-substituted 4- to 9-membered monocyclic or 7- to 10-membered bicyclic saturated heterocyclic group (wherein the binding site is any one carbon atom in the heterocyclic ring),

wherein the C₁₋₆ alkyl group, C₃₋₆ alkenyl group, C₃₋₆ alkynyl group, C₃₋₆ cycloalkyl group, C₆₋₆ cycloalkenyl group, 5- to 9-membered monocyclic or 7- to 10-membered bicyclic non-aromatic unsaturated heterocyclic group, and 4- to 9-membered monocyclic or 7- to 10-membered bicyclic saturated heterocyclic group may be independently and optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, aryl group, heteroaryl group, and halogen atom at each substitutable position thereof; and the aryl group and heteroaryl group may be independently and optionally substituted with one or more substituents independently-selected from the group consisting of halogen atom, hydroxy group, C₁₋₄ alkyl group, C₁₋₄ alkoxy group, C₁₋₄ haloalkyl group, C₁₋₄ haloalkoxy group, cyano group, nitro group, C₂₋₆ alkanoyl group, and an optionally-substituted amino group at each substitutable position thereof;
(4) —CONR¹⁷—OR¹⁸

wherein R¹⁷ and R¹⁸ are independently hydrogen atom, C₁₋₆ alkyl group, C₃₋₆ alkenyl group or C₃₋₆ alkynyl group;

### (5) —CONR¹⁸R²⁰, CSNR¹⁹R²⁰ and —SO₂NR¹⁹R²⁰

wherein R¹⁰ and R²⁰ are independently hydrogen atom or any group defined in the said R¹⁶, or

R¹⁹ and R²⁰ may be taken together with the adjacent nitrogen atom to form a saturated or unsaturated 4- to 8-membered monocyclic nitrogen-containing heterocyclic group comprising additional 0 to 2 heteroatoms independently-selected from the group consisting of 1 to 2 nitrogen atoms, 1 oxygen atom and 1 sulfur atom wherein the heterocyclic group may be optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, and halogen atom at each substitutable position thereof,

R¹⁴ and R¹⁵ are independently hydrogen atom, an optionally-substituted C₁₋₆ alkyl group, an optionally-substituted C₃₋₆ alkenyl group, an optionally-substituted C₃₋₆ alkynyl group, an optionally-substituted C₃₋₈ cycloalkyl group, an optionally-substituted C₅₋₈ cycloalkenyl group, an optionally-substituted aryl group, an optionally-substituted heteroaryl group, an optionally-substituted 5- to 9-membered monocyclic or 7- to 10-membered bicyclic non-aromatic unsaturated heterocyclic group (which is attached to the adjacent nitrogen atom via any one carbon atom in the heterocyclic group), an optionally-substituted 4- to 9-membered monocyclic or 7- to 10-membered bicyclic saturated heterocyclic group (which is attached to the adjacent nitrogen atom via any one carbon atom in the heterocyclic group), C₂₋₆ alkanoyl group, C₁₋₆ alkoxycarbonyl group, carbamoyl group, sulfamoyl group, or C₁₋₆ alkylsulfonyl group,

wherein the C₁₋₆ alkyl group, C₃₋₆ alkenyl group, C₃₋₆ alkynyl group, C₃₋₈ cycloalkyl group, C₅₋₈ cycloalkenyl group, 5- to 9-membered monocyclic or 7- to 10-membered bicyclic non-aromatic unsaturated heterocyclic group, 4- to 9-membered monocyclic or 7- to 10-membered bicyclic saturated heterocyclic group, C₂₋₆ alkanoyl group, C₁₋₆ alkoxycarbonyl group, and C₁₋₆ alkylsulfonyl group may be independently and optionally substituted with one or more substituents independently-selected from the group consisting of C₁₋₄ alkyl group, hydroxy group, C₁₋₄ alkoxy group, cyano group, oxo group, aryl group, heteroaryl group, and halogen atom at each substitutable position thereof; and the aryl group and heteroaryl group may be independently and optionally substituted with one or more substituents independently-selected from the group consisting of halogen atom, hydroxy group, C₁₋₄ alkyl group, C₁₋₄ alkoxy group, cyano group, nitro group, C₂₋₆ alkanoyl group, and an optionally-substituted amino group at each substitutable position thereof, or

R¹⁴ and R¹⁵ may be taken together with the adjacent nitrogen atom to form a saturated or unsaturated 4- to 9-membered monocyclic or 7- to 10-membered bicyclic nitrogen-containing heterocyclic group comprising additional 0 to 2 heteroatoms independently-selected from the group consisting of 1 to 2 nitrogen atoms, 1 oxygen atom and 1 sulfur atom wherein the heterocyclic group may be optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, and halogen atom at each substitutable position thereof,
(R¹²-1) to (R¹²-4) may optionally include an unsaturated bond(s) at an acceptable position(s) of the ring,
R⁸′ and R⁹′ are independently hydrogen atom, an optionally-substituted C₁₋₈ alkyl group, an optionally-substituted C₃₋₆ alkenyl group, an optionally-substituted C₃₋₈ alkynyl group, an optionally-substituted C₃₋₈ cycloalkyl group, an optionally-substituted C₅₋₈ cycloalkenyl group, an optionally-substituted aryl group, an optionally-substituted heteroaryl group, an optionally-substituted 5- to 9-membered monocyclic or 7- to 10-membered bicyclic non-aromatic unsaturated heterocyclic group (which is attached to the adjacent nitrogen atom via any one carbon atom in the heterocyclic group), or an optionally-substituted 4- to 9-membered monocyclic or 7- to 10-membered bicyclic saturated heterocyclic group (which is attached to the adjacent nitrogen atom via any one carbon atom in the heterocyclic group),

wherein the C₁₋₆ alkyl group, C₃₋₆ alkenyl group, C₃₋₆ alkynyl group, C₃₋₈ cycloalkyl group, C₅₋₈ cycloalkenyl group, 5- to 9-membered monocyclic or 7- to 10-membered bicyclic non-aromatic unsaturated heterocyclic group, and 4- to 9-membered monocyclic or 7- to 10-membered bicyclic saturated heterocyclic group may be independently and optionally substituted with one or more substituents independently-selected from the group consisting of C₁₋₄ alkyl group, hydroxy group, C₁₋₄ alkoxy group, C₁₋₄ haloalkoxy group, cyano group, oxo group, aryl group, heteroaryl group, aryloxy group, C₂₋₆ alkanoyl group, phenacyl group, and halogen atom at each substitutable position thereof; and the aryl group and heteroaryl group may be independently and optionally substituted with one or more substituents independently-selected from the group consisting of halogen atom, hydroxy group, C₁₋₄ alkyl group, C₁₋₄ alkoxy group, C₁₋₄ haloalkyl group, C₁₋₄ haloalkoxy group, cyano group, nitro group, C₂₋₆ alkanoyl group, and an optionally-substituted amino group at each substitutable position thereof, or a pair of R⁸ and R⁹, and a pair of R⁸′ and R⁹′ may be independently taken together with the adjacent nitrogen atom to form a saturated or unsaturated 4- to 9-membered monocyclic or 7- to 10-membered bicyclic nitrogen-containing heterocyclic group comprising additional 0 to 2 heteroatoms independently-selected from the group consisting of 1 to 2 nitrogen atoms, 1 oxygen atom and 1 sulfur atom wherein the nitrogen-containing heterocyclic group may be optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, and halogen atom at each substitutable position thereof,

R¹⁰, R¹⁰′, R¹¹ and R¹¹′ are independently hydrogen atom, halogen atom, hydroxy group, an optionally-substituted C₁₋₆ alkyl group, an optionally-substituted C₂₋₆ alkenyl group, an optionally-substituted C₂₋₆ alkynyl group, an optionally-substituted C₁₋₆ alkoxy group, cyano group, or an oxo group,

wherein the C₁₋₆ alkyl group, C₂₋₆ alkenyl group, C₂₋₆ alkynyl group, and C₁₋₆ alkoxy group may be independently and optionally substituted with one or more substituents independently-selected from the group consisting of C₁₋₄ alkyl group, hydroxy group, C₁₋₄ alkoxy group, C₁₋₄ haloalkoxy group, cyano group, oxo group, aryl group, heteroaryl group, aryloxy group, C₂₋₆ alkanoyl group, phenacyl group, and halogen atom at each substitutable position thereof, or

a pair of R¹⁰ and R¹¹, and a pair of R¹⁰′ and R¹¹′ may be independently taken together to form an optionally-substituted saturated or unsaturated 3- to 8-membered ring that may comprise 1 oxygen atom, which may be a bicyclic or a spiro compound with the ring to which the pair of R¹⁰ and R¹¹, or R¹⁰′ and R¹¹′ is attached,

wherein the saturated or unsaturated 3- to 8-membered ring may be optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, aryl group, heteroaryl group, aryloxy group, C₂₋₆ alkanoyl group, phenacyl group, and halogen atom at each substitutable position thereof,

r and r′ are independently an integer of 0 to 3,
s and s′ are independently an integer of 0 to 3,
t and t′ are independently 1 or 2,
v is an integer of 0 to 2,
provided that not both r and s are 0,
V is nitrogen atom or C—R¹ wherein R¹ is hydrogen atom, halogen atom, an optionally-substituted C₁₋₆ alkyl group, an optionally-substituted C₂₋₆ alkenyl group, an optionally-substituted C₂₋₆ alkynyl group, an optionally-substituted C₃₋₈ cycloalkyl group, an optionally-substituted C₅₋₈ cycloalkenyl group, an optionally-substituted aryl group, or an optionally-substituted heteroaryl group,

wherein the C₁₋₆ alkyl group, C₂₋₆ alkenyl group, C₂₋₆ alkynyl group, C₃₋₈ cycloalkyl group, and C₅₋₈ cycloalkenyl group may be independently and optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, aryl group, heteroaryl group, aryloxy group, C₂₋₆ alkanoyl group, phenacyl group, and halogen atom at each substitutable position thereof; and the aryl group and heteroaryl group may be independently and optionally substituted with one or more substituents independently-selected from the group consisting of halogen atom, hydroxy group, C₁₋₄ alkyl group, C₁₋₄ alkoxy group, C₁₋₄ haloalkyl group, C₁₋₄ haloalkoxy group, cyano group, nitro group, C₂₋₆ alkanoyl group, and an optionally-substituted amino group at each substitutable position thereof,

W is nitrogen atom or C—R² wherein R² is hydrogen atom, halogen atom, hydroxy group, an optionally-substituted C₁₋₆ alkyl group, an optionally-substituted C₂₋₆ alkenyl group, an optionally-substituted C₂₋₆ alkynyl group, an optionally-substituted C₃₋₈ cycloalkyl group, an optionally-substituted C₅₋₈ cycloalkenyl group, an optionally-substituted C₁₋₆ alkoxy group, an optionally-substituted C₁₋₄ haloalkyl group, an optionally-substituted C₁₋₄ haloalkoxy group, cyano group, nitro group, an optionally-substituted aryl group, an optionally-substituted heteroaryl group, or an optionally-substituted amino group,

wherein the C₁₋₆ alkyl group, C₂₋₆ alkenyl group, C₂₋₆ alkynyl group, C₃₋₈ cycloalkyl group, C₅₋₈ cycloalkenyl group, C₁₋₆ alkoxy group, C₁₋₄ haloalkyl group, and C₁₋₄ haloalkoxy group may be independently and optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, aryl group, heteroaryl group, aryloxy group, C₂₋₆ alkanoyl group, phenacyl group, and halogen atom at each substitutable position thereof; and the aryl group and heteroaryl group may be independently and optionally substituted with one or more substituents independently-selected from the group consisting of halogen atom, hydroxy group, C₁₋₄ alkyl group, C₁₋₄ alkoxy group, C₁₋₄ haloalkyl group, C₁₋₄ haloalkoxy group, cyano group, nitro group, C₂₋₆ alkanoyl group, and an optionally-substituted amino group at each substitutable position thereof,

provided that when V is C—R¹, W is nitrogen atom, and when V is nitrogen atom, W is C—R²,

U is carbon atom or nitrogen atom,
X, Y and Z are independently selected from the group consisting of oxygen atom, nitrogen atom, sulfur atom and carbon atom, provided that at least one of X, Y and Z is oxygen atom, sulfur atom, or nitrogen atom,
R³ is hydrogen atom, halogen atom, an optionally-substituted C₁₋₆ alkyl group, an optionally-substituted C₂₋₆ alkenyl group, an optionally-substituted C₂₋₆ alkynyl group, an optionally-substituted C₃₋₈ cycloalkyl group, an optionally-substituted C₅₋₈ cycloalkenyl group, an optionally-substituted C₁₋₆ alkoxy group, an optionally-substituted C₁₋₄ haloalkyl group, an optionally-substituted C₁₋₄ haloalkoxy group, cyano group, nitro group, an optionally-substituted aryl group, an optionally-substituted heteroaryl group, an optionally-substituted 5- to 9-membered monocyclic or 7- to 10-membered bicyclic non-aromatic unsaturated heterocyclic group, or an optionally-substituted 4- to 9-membered monocyclic or 7- to 10-membered bicyclic saturated heterocyclic group,

wherein the C₁₋₆ alkyl group, C₂₋₆ alkenyl group, C₂₋₆ alkynyl group, C₃₋₆ cycloalkyl group, C₅₋₆ cycloalkenyl group, alkoxy group, C₁₋₄ haloalkyl group, C₁₋₄ haloalkoxy group, 5- to 9-membered monocyclic or 7- to 10-membered bicyclic non-aromatic unsaturated heterocyclic group, and 4- to 9-membered monocyclic or 7- to 10-membered bicyclic saturated heterocyclic group may be independently and optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, aryl group, heteroaryl group, aryloxy group, C₂₋₆ alkanoyl group, phenacyl group, and halogen atom at each substitutable position thereof; and the aryl group and heteroaryl group may be independently and optionally substituted with one or more substituents independently-selected from the group consisting of halogen atom, hydroxy group, C₁₋₄ alkyl group, C₁₋₄ alkoxy group, C₁₋₄ haloalkyl group, C₁₋₄ haloalkoxy group, cyano group, nitro group, C₂₋₆ alkanoyl group, and an optionally-substituted amino group at each substitutable position thereof,

R⁴ is hydrogen atom, halogen atom, hydroxy group, an optionally-substituted C₁₋₆ alkyl group, an optionally-substituted C₂₋₆ alkenyl group, an optionally-substituted C₂₋₆ alkynyl group, an optionally-substituted C₃₋₈ cycloalkyl group, an optionally-substituted C₅₋₈ cycloalkenyl group, an optionally-substituted C₁₋₆ alkoxy group, an optionally-substituted C₁₋₄ haloalkyl group, an optionally-substituted C₁₋₄ haloalkoxy group, cyano group, nitro group, an optionally-substituted aryl group, an optionally-substituted heteroaryl group, or an optionally-substituted amino group,

wherein the C₁₋₆ alkyl group, C₂₋₆ alkenyl group, C₂₋₆ alkynyl group, C₃₋₈ cycloalkyl group, C₅₋₈ cycloalkenyl group, C₁₋₆ alkoxy group, C₁₋₄ haloalkyl group, and C₁₋₄ haloalkoxy group may be independently and optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, aryl group, heteroaryl group, aryloxy group, C₂₋₆ alkanoyl group, phenacyl group, and halogen atom at each substitutable position thereof; and the aryl group and heteroaryl group may be independently and optionally substituted with one or more substituents independently-selected from the group consisting of halogen atom, hydroxy group, C₁₋₄ alkyl group, C₁₋₄ alkoxy group, C₁₋₄ haloalkyl group, C₁₋₄ haloalkoxy group, cyano group, nitro group, C₂₋₆ alkanoyl group, and an optionally-substituted amino group at each substitutable position thereof, or

R³ and R⁴ may be taken together to form a saturated or unsaturated 6- to 9-membered ring optionally comprising 1 oxygen atom wherein the ring may be optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, and halogen atom at each substitutable position thereof, and
R⁵ and R⁶ are independently hydrogen atom, halogen atom, hydroxy group, an optionally-substituted C₁₋₆ alkyl group, an optionally-substituted C₂₋₆ alkenyl group, an optionally-substituted C₂₋₆ alkynyl group, an optionally-substituted C₃₋₈ cycloalkyl group, an optionally-substituted C₈₋₈ cycloalkenyl group, an optionally-substituted C₁₋₆ alkoxy group, an optionally-substituted C₁₋₄ haloalkyl group, an optionally-substituted C₁₋₄ haloalkoxy group, cyano group, nitro group, an optionally-substituted aryl group, an optionally-substituted heteroaryl group, or an optionally-substituted amino group,

wherein the C₁₋₆ alkyl group, C₂₋₆ alkenyl group, C₂₋₆ alkynyl group, C₃₋₈ cycloalkyl group, C₈₋₈ cycloalkenyl group, C₁₋₆ alkoxy group, C₁₋₄ haloalkyl group, and C₁₋₄ haloalkoxy group may be independently and optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, aryl group, heteroaryl group, aryloxy group, C₂₋₆ alkanoyl group, phenacyl group, and halogen atom at each substitutable position thereof; and the aryl group and heteroaryl group may be independently and optionally substituted with one or more substituents independently-selected from the group consisting of halogen atom, hydroxy group, C₁₋₄ alkyl group, C₁₋₄ alkoxy group, C₁₋₄ haloalkyl group, C₁₋₄ haloalkoxy group, cyano group, nitro group, C₂₋₆ alkanoyl group, and an optionally-substituted amino group at each substitutable position thereof.

### Term 2

A compound of Formula (1):

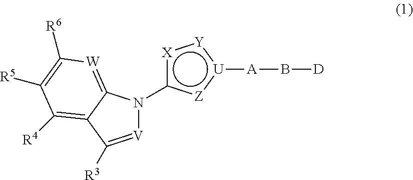

or a pharmaceutically acceptable salt thereof wherein
A is the following Formula (A-1), Formula (A-2), Formula (A-3), or Formula (A-4):

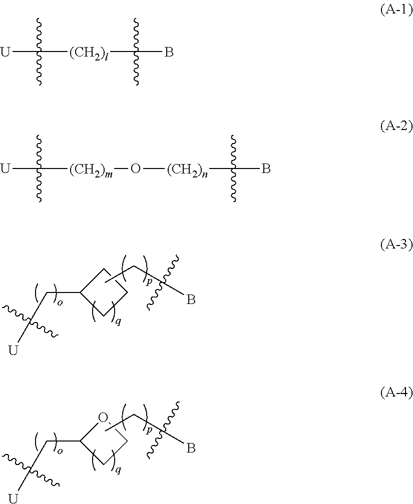

wherein
l is an integer of 0 to 4,
m is an integer of 0 to 2,
n is an integer of 0 to 2,
o and p are independently an integer of 0 or 1,
q is an integer of 0 to 5,
(A-1) to (A-4) may be independently and optionally substituted with one or more substituents independently-selected from the group consisting of C₁₋₆ alkyl group, C₂₋₆ alkenyl group, C₂₋₆ alkynyl group, hydroxy group, C₁₋₆ alkoxy group, oxo group and halogen atom at each substitutable position thereof,
B is the following Formula (B-1), Formula (B-2), or Formula (B-3):

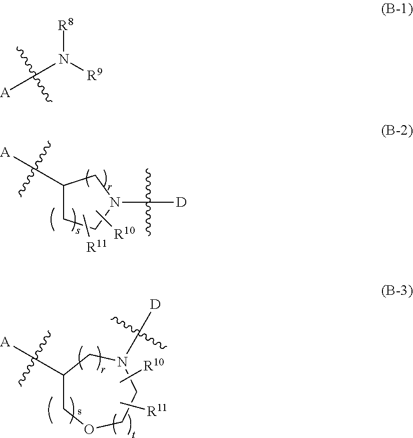

wherein (B-2) and (B-3) may optionally include an unsaturated bond(s) at an acceptable position(s) of the ring, and D is absent when B is Formula (B-1),
D is independently a group selected from the group consisting of the following (1) and (2):
(1) hydrogen atom, an optionally-substituted C₁₋₆ alkyl group, an optionally-substituted C₃₋₆ alkenyl group, an optionally-substituted C₃₋₆ alkynyl group, an optionally-substituted C₃₋₈ monocyclic, C₇₋₁₀ bicyclic or C₇₋₁₂ tricyclic cycloalkyl group, and an optionally-substituted C₅₋₈ monocyclic or C₇₋₁₀ bicyclic cycloalkenyl group

wherein the C₁₋₆ alkyl group, C₃₋₆ alkenyl group, C₃₋₆ alkynyl group, C₃₋₈ monocyclic, C₇₋₁₀ bicyclic or C₇₋₁₂ tricyclic cycloalkyl group, and C₅₋₈ monocyclic or C₇₋₁₀ bicyclic cycloalkenyl group may be independently and optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, aryl group, heteroaryl group, aryloxy group, C₂₋₆ alkanoyl group, phenacyl group, and halogen atom at each substitutable position thereof;

(2) —(CH₂)ᵤ—R¹²

wherein u is an integer of 0 to 4 provided that when u is an integer of 1 to 4, the alkylene chain may be optionally substituted with one or more substituents independently-selected from the group consisting of C₁₋₆ alkyl group, C₂₋₆ alkenyl group, C₂₋₆ alkynyl group, hydroxy group, C₁₋₆ alkoxy group, oxo group, and halogen atom,

R¹² is the following Formula (R¹²-1), Formula (R¹²-2), Formula (R¹²-3), Formula (R¹²-4), Formula (R¹²-5), Formula (R¹²-6), Formula (R¹²-7), or Formula (R¹²-8):

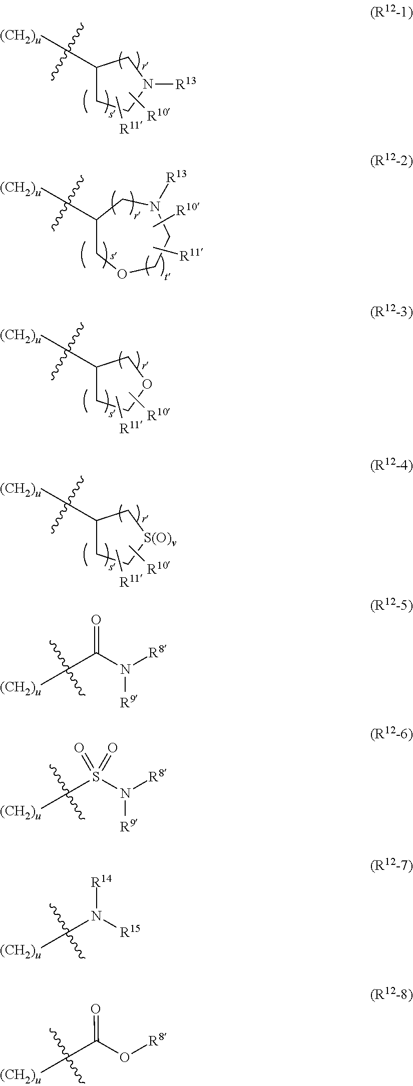

wherein R¹³ is a group selected from the group consisting of the following (1) to (5):
(1) hydrogen atom and formyl group;
(2) an optionally-substituted C₁₋₆ alkyl group, an optionally-substituted C₃₋₆ alkenyl group, an optionally-substituted C₃₋₆ alkynyl group, an optionally-substituted C₃₋₈ cycloalkyl group, and an optionally-substituted C₅₋₈ cycloalkenyl group

wherein the C₁₋₆ alkyl group, C₃₋₆ alkenyl group, C₃₋₆ alkynyl group, C₃₋₈ cycloalkyl group, and C₅₋₈ cycloalkenyl group may be independently and optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, and halogen atom at each substitutable position thereof;
(3) —COR¹⁶, —CSR¹⁶, —SO₂R¹⁶, —CO—COR¹⁶, COOR¹⁶, and —CO—COOR¹⁶

wherein R¹⁶ is an optionally-substituted C₁₋₆ alkyl group, an optionally-substituted C₃₋₆ alkenyl group, an optionally-substituted C₃₋₆ alkynyl group, an optionally-substituted C₃₋₈ cycloalkyl group, an optionally-substituted C₅₋₈ cycloalkenyl group, an optionally-substituted aryl group, an optionally-substituted heteroaryl group, an optionally-substituted 5- to 9-membered monocyclic or 7- to 10-membered bicyclic non-aromatic unsaturated heterocyclic group (wherein the binding site is any one carbon atom in the heterocyclic ring), or an optionally-substituted 4- to 9-membered monocyclic or 7- to 10-membered bicyclic saturated heterocyclic group (wherein the binding site is any one carbon atom in the heterocyclic ring),

wherein the C₁₋₆ alkyl group, C₃₋₆ alkenyl group, C₃₋₆ alkynyl group, C₃₋₈ cycloalkyl group, C₅₋₈ cycloalkenyl group, 5- to 9-membered monocyclic or 7- to 10-membered bicyclic non-aromatic unsaturated heterocyclic group, and 4- to 9-membered monocyclic or 7- to 10-membered bicyclic saturated heterocyclic group may be independently and optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, aryl group, heteroaryl group, and halogen atom at each substitutable position thereof; and the aryl group and heteroaryl group may be independently and optionally substituted with one or more substituents independently-selected from the group consisting of halogen atom, hydroxy group, C₁₋₄ alkyl group, C₁₋₄ alkoxy group, C₁₋₄ haloalkyl group, C₁₋₄ haloalkoxy group, cyano group, nitro group, C₂₋₆ alkanoyl group, and an optionally-substituted amino group at each substitutable position thereof;

### (4) —CONR¹⁷—OR¹⁸

wherein R¹⁷ and R¹⁸ are independently hydrogen atom, C₁₋₆ alkyl group, C₃₋₆ alkenyl group or C₃₋₆ alkynyl group;

### (5) —CONR¹⁹R²⁰CSNR¹⁹R²⁰ and —SO₂NR¹⁹R²⁰

wherein R¹⁸ and R²⁰ are independently hydrogen atom or any group defined in the said R¹⁸, or

R¹⁸ and R²⁰ may be taken together with the adjacent nitrogen atom to form a saturated or unsaturated 4- to 8-membered monocyclic nitrogen-containing heterocyclic group comprising additional 0 to 2 heteroatoms independently-selected from the group consisting of 1 to 2 nitrogen atoms, 1 oxygen atom and 1 sulfur atom wherein the heterocyclic group may be optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, and halogen atom at each substitutable position thereof,

R¹⁴ and R¹⁵ are independently hydrogen atom, an optionally-substituted C₁₋₆ alkyl group, an optionally-substituted C₃₋₆ alkenyl group, an optionally-substituted C₃₋₆ alkynyl group, an optionally-substituted C₃₋₈ cycloalkyl group, an optionally-substituted C₅₋₈ cycloalkenyl group, an optionally-substituted aryl group, an optionally-substituted heteroaryl group, an optionally-substituted 5- to 9-membered monocyclic or 7- to 10-membered bicyclic non-aromatic unsaturated heterocyclic group (which is attached to the adjacent nitrogen atom via any one carbon atom in the heterocyclic group), an optionally-substituted 4- to 9-membered monocyclic or 7- to 10-membered bicyclic saturated heterocyclic group (which is attached to the adjacent nitrogen atom via any one carbon atom in the heterocyclic group), C₂₋₆ alkanoyl group, C₁₋₆ alkoxycarbonyl group, carbamoyl group, sulfamoyl group, or C₁₋₆ alkylsulfonyl group,

wherein the C₁₋₆ alkyl group, C₃₋₆ alkenyl group, C₃₋₆ alkynyl group, C₃₋₈ cycloalkyl group, C₅₋₈ cycloalkenyl group, 5- to 9-membered monocyclic or 7- to 10-membered bicyclic non-aromatic unsaturated heterocyclic group, 4- to 9-membered monocyclic or 7- to 10-membered bicyclic saturated heterocyclic group, C₂₋₆ alkanoyl group, C₁₋₆ alkoxycarbonyl group, and C₁₋₆ alkylsulfonyl group may be independently and optionally substituted with one or more substituents independently-selected from the group consisting of C₁₋₄ alkyl group, hydroxy group, C₁₋₄ alkoxy group, cyano group, oxo group, aryl group, heteroaryl group, and halogen atom at each substitutable position thereof; and the aryl group and heteroaryl group may be independently and optionally substituted with one or more substituents independently-selected from the group consisting of halogen atom, hydroxy group, C₁₋₄ alkyl group, C₁₋₄ alkoxy group, cyano group, nitro group, C₂₋₆ alkanoyl group, and an optionally-substituted amino group at each substitutable position thereof, or

R¹⁴ and R¹⁵ may be taken together with the adjacent nitrogen atom to form a saturated or unsaturated 4- to 9-membered monocyclic or 7- to 10-membered bicyclic nitrogen-containing heterocyclic group comprising additional 0 to 2 heteroatoms independently-selected from the group consisting of 1 to 2 nitrogen atoms, 1 oxygen atom and 1 sulfur atom wherein the heterocyclic group may be optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, and halogen atom at each substitutable position thereof,
(R¹²-1) to (R¹²-4) may optionally include an unsaturated bond(s) at an acceptable position(s) of the ring,
R⁸, R⁸′, R⁹ and R⁹′ are independently hydrogen atom, an optionally-substituted C₁₋₆ alkyl group, an optionally-substituted C₃₋₆ alkenyl group, an optionally-substituted C₃₋₆ alkynyl group, an optionally-substituted C₃₋₈ cycloalkyl group, an optionally-substituted C₅₋₈ cycloalkenyl group, an optionally-substituted aryl group, an optionally-substituted heteroaryl group, an optionally-substituted 5- to 9-membered monocyclic or 7- to 10-membered bicyclic non-aromatic unsaturated heterocyclic group (which is attached to the adjacent nitrogen atom via any one carbon atom in the heterocyclic group), or an optionally-substituted 4- to 9-membered monocyclic or 7- to 10-membered bicyclic saturated heterocyclic group (which is attached to the adjacent nitrogen atom via any one carbon atom in the heterocyclic group),

wherein the C₁₋₆ alkyl group, C₃₋₆ alkenyl group, C₃₋₆ alkynyl group, C₃₋₈ cycloalkyl group, C₅₋₈ cycloalkenyl group, 5- to 9-membered monocyclic or 7- to 10-membered bicyclic non-aromatic unsaturated heterocyclic group, and 4- to 9-membered monocyclic or 7- to 10-membered bicyclic saturated heterocyclic group may be independently and optionally substituted with one or more substituents independently-selected from the group consisting of C₁₋₄ alkyl group, hydroxy group, C₁₋₄ alkoxy group, C₁₋₄ haloalkoxy group, cyano group, oxo group, aryl group, heteroaryl group, aryloxy group, C₂₋₆ alkanoyl group, phenacyl group, and halogen atom at each substitutable position thereof; and the aryl group and heteroaryl group may be independently and optionally substituted with one or more substituents independently-selected from the group consisting of halogen atom, hydroxy group, C₁₋₄ alkyl group, C₁₋₄ alkoxy group, C₁₋₄ haloalkyl group, C₁₋₄ haloalkoxy group, cyano group, nitro group, C₂₋₆ alkanoyl group, and an optionally-substituted amino group at each substitutable position thereof, or a pair of R⁸ and R⁹, and a pair of R⁸′ and R⁹′ may be independently taken together with the adjacent nitrogen atom to form a saturated or unsaturated 4- to 9-membered monocyclic or 7- to 10-membered bicyclic nitrogen-containing heterocyclic group comprising additional 0 to 2 heteroatoms independently-selected from the group consisting of 1 to 2 nitrogen atoms, 1 oxygen atom and 1 sulfur atom wherein the nitrogen-containing heterocyclic group may be optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, and halogen atom at each substitutable position thereof,

R¹⁰, R¹⁰′, R¹¹ and R¹¹′ are independently hydrogen atom, halogen atom, hydroxy group, an optionally-substituted C₁₋₆ alkyl group, an optionally-substituted C₂₋₆ alkenyl group, an optionally-substituted C₂₋₆ alkynyl group, an optionally-substituted C₁₋₆ alkoxy group, cyano group, or an oxo group,

wherein the C₁₋₆ alkyl group, C₂₋₆ alkenyl group, C₂₋₆ alkynyl group, and C₁₋₆ alkoxy group may be independently and optionally substituted with one or more substituents independently-selected from the group consisting of C₁₋₄ alkyl group, hydroxy group, C₁₋₄ alkoxy group, C₁₋₄ haloalkoxy group, cyano group, oxo group, aryl group, heteroaryl group, aryloxy group, C₂₋₆ alkanoyl group, phenacyl group, and halogen atom at each substitutable position thereof, or

a pair of R¹⁰ and R¹¹, and a pair of R¹⁰′ and R¹¹′ may be independently taken together to form an optionally-substituted saturated or unsaturated 3- to 8-membered ring that may comprise 1 oxygen atom, which may be a bicyclic or a spiro compound with the ring to which the pair of R¹⁰ and R¹¹ is attached,

wherein the saturated or unsaturated 3- to 8-membered ring may be optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, aryl group, heteroaryl group, aryloxy group, C₂₋₆ alkanoyl group, phenacyl group, and halogen atom at each substitutable position thereof,

r and r′ are independently an integer of 0 to 3,
s and s′ are independently an integer of 0 to 3,
t and t′ are independently 1 or 2,
v is an integer of 0 to 2,
provided that not both r and s are 0,
V is nitrogen atom or C—R¹ wherein R¹ is hydrogen atom, halogen atom, an optionally-substituted C₁₋₆ alkyl group, an optionally-substituted C₂₋₆ alkenyl group, an optionally-substituted C₂₋₆ alkynyl group, an optionally-substituted C₃₋₈ cycloalkyl group, an optionally-substituted C₅₋₈ cycloalkenyl group, an optionally-substituted aryl group, or an optionally-substituted heteroaryl group,

wherein the C₁₋₆ alkyl group, C₂₋₆ alkenyl group, C₂₋₆ alkynyl group, C₃₋₈ cycloalkyl group, and C₅₋₈ cycloalkenyl group may be independently and optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, aryl group, heteroaryl group, aryloxy group, C₂₋₆ alkanoyl group, phenacyl group, and halogen atom at each substitutable position thereof; and the aryl group and heteroaryl group may be independently and optionally substituted with one or more substituents independently-selected from the group consisting of halogen atom, hydroxy group, C₁₋₄ alkyl group, C₁₋₄ alkoxy group, C₁₋₄ haloalkyl group, C₁₋₄ haloalkoxy group, cyano group, nitro group, C₂₋₆ alkanoyl group, and an optionally-substituted amino group at each substitutable position thereof,

W is nitrogen atom or C—R² wherein R² is hydrogen atom, halogen atom, hydroxy group, an optionally-substituted C₁₋₆ alkyl group, an optionally-substituted C₂₋₆ alkenyl group, an optionally-substituted C₂₋₆ alkynyl group, an optionally-substituted C₃₋₈ cycloalkyl group, an optionally-substituted C₅₋₈ cycloalkenyl group, an optionally-substituted C₁₋₆ alkoxy group, an optionally-substituted C₁₋₄ haloalkyl group, an optionally-substituted C₁₋₄ haloalkoxy group, cyano group, nitro group, an optionally-substituted aryl group, an optionally-substituted heteroaryl group, or an optionally-substituted amino group,

wherein the C₁₋₆ alkyl group, C₂₋₆ alkenyl group, C₂₋₆ alkynyl group, C₃₋₈ cycloalkyl group, C₅₋₈ cycloalkenyl group, C₁₋₆ alkoxy group, C₁₋₄ haloalkyl group, and C₁₋₄ haloalkoxy group may be independently and optionally substituted with one or more substituents independently-selected from the group consisting of C₁₋₄ alkyl group, hydroxy group, C₁₋₄ alkoxy group, haloalkyl group, C₁₋₄ haloalkoxy group, cyano group, oxo group, aryl group, heteroaryl group, aryloxy group, C₂₋₆ alkanoyl group, phenacyl group, and halogen atom at each substitutable position thereof; and the aryl group and heteroaryl group may be independently and optionally substituted with one or more substituents independently-selected from the group consisting of halogen atom, hydroxy group, C₁₋₄ alkyl group, C₁₋₄ alkoxy group, C₁₋₄ haloalkyl group, C₁₋₄ haloalkoxy group, cyano group, nitro group, C₂₋₆ alkanoyl group, and an optionally-substituted amino group at each substitutable position thereof,

provided that when V is C—R¹, W is nitrogen atom, and when V is nitrogen atom, W is C—R²,

U is carbon atom or nitrogen atom,
X, Y and Z are independently selected from the group consisting of oxygen atom, nitrogen atom, sulfur atom and carbon atom, provided that at least one of X, Y and Z is oxygen atom, sulfur atom, or nitrogen atom,
R³ is hydrogen atom, halogen atom, an optionally-substituted C₁₋₆ alkyl group, an optionally-substituted C₂₋₆ alkenyl group, an optionally-substituted C₂₋₆ alkynyl group, an optionally-substituted C₃₋₈ cycloalkyl group, an optionally-substituted C₅₋₈ cycloalkenyl group, an optionally-substituted C₁₋₆ alkoxy group, an optionally-substituted C₁₋₄ haloalkyl group, an optionally-substituted C₁₋₄ haloalkoxy group, cyano group, nitro group, an optionally-substituted aryl group, an optionally-substituted heteroaryl group, an optionally-substituted 5- to 9-membered monocyclic or 7- to 10-membered bicyclic non-aromatic unsaturated heterocyclic group, or an optionally-substituted 4- to 9-membered monocyclic or 7- to 10-membered bicyclic saturated heterocyclic group,

wherein the C₁₋₆ alkyl group, C₂₋₆ alkenyl group, C₂₋₆ alkynyl group, C₃₋₈ cycloalkyl group, C₅₋₈ cycloalkenyl group, C₁₋₆ alkoxy group, C₁₋₄ haloalkyl group, C₁₋₄ haloalkoxy group, 5- to 9-membered monocyclic or 7- to 10-membered bicyclic non-aromatic unsaturated heterocyclic group, and 4- to 9-membered monocyclic or 7- to 10-membered bicyclic saturated heterocyclic group may be independently and optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, aryl group, heteroaryl group, aryloxy group, C₂₋₆ alkanoyl group, phenacyl group, and halogen atom at each substitutable position thereof; and the aryl group and heteroaryl group may be independently and optionally substituted with one or more substituents independently-selected from the group consisting of halogen atom, hydroxy group, C₁₋₄ alkyl group, C₁₋₄ alkoxy group, C₁₋₄ haloalkyl group, C₁₋₄ haloalkoxy group, cyano group, nitro group, C₂₋₆ alkanoyl group, and an optionally-substituted amino group at each substitutable position thereof,

R⁴ is hydrogen atom, halogen atom, hydroxy group, an optionally-substituted C₁₋₆ alkyl group, an optionally-substituted C₂₋₆ alkenyl group, an optionally-substituted C₂₋₆ alkynyl group, an optionally-substituted C₃₋₈ cycloalkyl group, an optionally-substituted C₅₋₈ cycloalkenyl group, an optionally-substituted C₁₋₆ alkoxy group, an optionally-substituted C₁₋₄ haloalkyl group, an optionally-substituted C₁₋₄ haloalkoxy group, cyano group, nitro group, an optionally-substituted aryl group, an optionally-substituted heteroaryl group, or an optionally-substituted amino group,

wherein the C₁₋₆ alkyl group, C₂₋₆ alkenyl group, C₂₋₆ alkynyl group, C₃₋₈ cycloalkyl group, C₅₋₈ cycloalkenyl group, C₁₋₆ alkoxy group, C₁₋₄ haloalkyl group, and C₁₋₄ haloalkoxy group may be independently and optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, aryl group, heteroaryl group, aryloxy group, C₂₋₆ alkanoyl group, phenacyl group, and halogen atom at each substitutable position thereof; and the aryl group and heteroaryl group may be independently and optionally substituted with one or more substituents independently-selected from the group consisting of halogen atom, hydroxy group, C₁₋₄ alkyl group, C₁₋₄ alkoxy group, C₁₋₄ haloalkyl group, C₁₋₄ haloalkoxy group, cyano group, nitro group, C₂₋₆ alkanoyl group, and an optionally-substituted amino group at each substitutable position thereof, or

R³ and R⁴ may be taken together to form a saturated or unsaturated 6- to 9-membered ring optionally comprising 1 oxygen atom wherein the ring may be optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, and halogen atom at each substitutable position thereof, and
R⁵ and R⁶ are independently hydrogen atom, halogen atom, hydroxy group, an optionally-substituted C₁₋₆ alkyl group, an optionally-substituted C₂₋₆ alkenyl group, an optionally-substituted C₂₋₆ alkynyl group, an optionally-substituted C₃₋₈ cycloalkyl group, an optionally-substituted C₅₋₈ cycloalkenyl group, an optionally-substituted C₁₋₆ alkoxy group, an optionally-substituted C₁₋₄ haloalkyl group, an optionally-substituted C₁₋₄ haloalkoxy group, cyano group, nitro group, an optionally-substituted aryl group, an optionally-substituted heteroaryl group, or an optionally-substituted amino group,

wherein the C₁₋₆ alkyl group, C₂₋₆ alkenyl group, C₂₋₆ alkynyl group, C₂₋₈ cycloalkyl group, C₆₋₈ cycloalkenyl group, C₁₋₆ alkoxy group, haloalkyl group, and C₁₋₄ haloalkoxy group may be independently and optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, aryl group, heteroaryl group, halogen atom at each substitutable position thereof; and the aryl group and heteroaryl group may be independently and optionally substituted with one or more substituents independently-selected from the group consisting of halogen atom, hydroxy group, C₁₋₄ alkyl group, C₁₋₄ alkoxy group, C₁₋₄ haloalkyl group, C₁₋₄ haloalkoxy group, cyano group, nitro group, C₂₋₆ alkanoyl group, and an optionally-substituted amino group at each substitutable position thereof.

### Term 3

The compound of Term 2 or a pharmaceutically acceptable salt thereof wherein the Formulae (A-1) to (A-4) may be independently and optionally substituted with one or more substituents independently-selected from the group consisting of C₁₋₆ alkyl group, C₂₋₆ alkenyl group, C₂₋₆ alkynyl group, hydroxy group, C₁₋₆ alkoxy group, and halogen atom at each substitutable position thereof.

### Term 4

The compound of any one of Terms 1 to 3 or a pharmaceutically acceptable salt thereof wherein V is nitrogen atom and W is C—R².

### Term 5

The compound of any one of Terms 1 to 4 or a pharmaceutically acceptable salt thereof wherein R³ is hydrogen atom, halogen atom, an optionally-substituted C₁₋₆ alkyl group, an optionally-substituted C₂₋₆ alkenyl group, an optionally-substituted C₂₋₆ alkynyl group, an optionally-substituted C₃₋₈ cycloalkyl group, or an optionally-substituted C₅₋₈ cycloalkenyl group.

### Term 6

The compound of any one of Terms 1 to 5 or a pharmaceutically acceptable salt thereof wherein R⁴ and R⁵ are hydrogen atom, and R² and R⁶ are independently hydrogen atom, halogen atom, an optionally-substituted C₁₋₆ alkyl group, an optionally-substituted C₁₋₆ alkoxy group, an optionally-substituted C₁₋₄ haloalkyl group, an optionally-substituted C₁₋₄ haloalkoxy group, or cyano group.

### Term 7

The compound of any one of Terms 1 to 6 or a pharmaceutically acceptable salt thereof wherein U is carbon atom.

### Term 8

The compound of any one of Terms 1 to 7 or a pharmaceutically acceptable salt thereof wherein X is nitrogen atom, Y is oxygen atom, and Z is nitrogen atom.

### Term 9

The compound of any one of Terms 1 to 8 or a pharmaceutically acceptable salt thereof wherein A is (A-1), and 1 is an integer of 0 or 1.

### Term 10

The compound of any one of Terms 1 to 9 or a pharmaceutically acceptable salt thereof wherein B is (B-2), s is an integer of 1, and r is an integer of 1 or 2.

### Term 11

The compound of any one of Terms 1 to 10 which has a chemical structure of Formula (12):

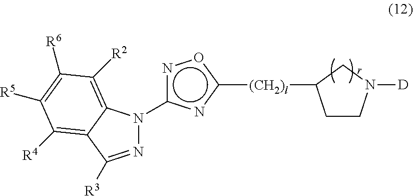

or a pharmaceutically acceptable salt thereof.

### Term 12

The compound of any one of Terms 1 to 11 or a pharmaceutically acceptable salt thereof wherein D is hydrogen atom, an optionally-substituted C₁₋₆ alkyl group, or an optionally-substituted C₃₋₈ monocyclic, C₇₋₁₀ bicyclic or C₇₋₁₂ tricyclic cycloalkyl group.

### Term 13

The compound of any one of Terms 1 to 11 or a pharmaceutically acceptable salt thereof wherein D is —(CH₂)ᵤ—R¹², and R¹² is Formula (R¹²-3).

### Term 14

The compound of any one of Terms 1 to 11 or a pharmaceutically acceptable salt thereof wherein D is —(CH₂)ᵤ—R¹², and R¹² is Formula (R¹²-1).

### Term 15

The compound of any one of Terms 1 to 8 or a pharmaceutically acceptable salt thereof wherein A is (A-3), is an integer of 0, p is an integer of 0, q is an integer of either 1 or 3, and B is (B-1).

### Term 16

The compound of any one of Terms 1 to 8 and 15 which has a chemical structure of Formula (13):

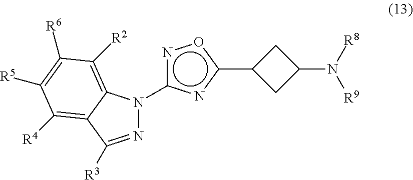

or a pharmaceutically acceptable salt thereof.

### Term 17

The compound of any one of Terms 1 to 11 and 14 which has a chemical structure of Formula (11):

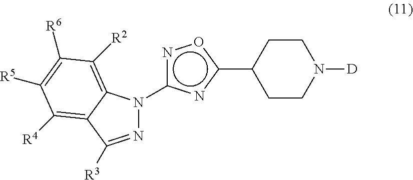

or a pharmaceutically acceptable salt thereof.

### Term 18

The compound of Term 1 which is selected from the group consisting of the following compounds or a pharmaceutically acceptable salt thereof:
(01) 1-{5-[1-(3-methoxypropyl)piperidin-4-yl]-1,2,4-oxadiazol-3-yl}-3-(propan-2-yl)-1H-indazole,
(02) 3-ethyl-1-{5-[1-(3-methoxypropyl)piperidin-4-yl]-1,2,4-oxadiazol-3-yl}-1H-indazole,
(03) 3-cyclopropyl-1-{5-[1-(3-methoxypropyl)piperidin-4-yl]-1,2,4-oxadiazol-3-yl}-1H-indazole,
(04) 3-ethyl-6-fluoro-1-{5-[1-(3-methoxypropyl)piperidin-4-yl]-1,2,4-oxadiazol-3-yl}-1H-indazole,
(05) 3-ethyl-7-fluoro-1-{5-[1-(3-methoxypropyl)piperidin-4-yl]-1,2,4-oxadiazol-3-yl}-1H-indazole,
(06) 1-{5-[1-(2-methylpropyl)piperidin-4-yl]-1,2,4-oxadiazol-3-yl}-3-(propan-2-yl)-1H-indazole,
(07) 1-{5-[1-(butan-2-yl)piperidin-4-yl]-1,2,4-oxadiazol-3-yl}-3-ethyl-1H-indazole,
(08) 1-{5-[1-(butan-2-yl)piperidin-4-yl]-1,2,4-oxadiazol-3-yl}-3-cyclopropyl-1H-indazole,
(09) 3-ethyl-1-{5-[1-(2-methylpropyl)piperidin-4-yl]-1,2,4-oxadiazol-3-yl}-1H-indazole,
(10) 1-{5-[1-(cyclopropylmethyl)piperidin-4-yl]-1,2,4-oxadiazol-3-yl}-3-ethyl-1H-indazole,
(11) 1-{5-[1-(butan-2-yl)piperidin-4-yl]-1,2,4-oxadiazol-3-yl}-3-cyclobutyl-1H-indazole,
(12) 3-cyclobutyl-1-{5-[1-(2-methylpropyl)piperidin-4-yl]-1,2,4-oxadiazol-3-yl}-1H-indazole,
(13) 3-(propan-2-yl)-1-[5-(1-propylpiperidin-4-yl)-1,2,4-oxadiazol-3-yl]-1H-indazole,
(14) 3-ethyl-6-fluoro-1-(5-{1-[2-(tetrahydrofuran-2-yl)ethyl]piperidin-4-yl}-1,2,4-oxadiazol-3-yl)-1H-indazole,
(15) 3-ethyl-1-{5-[1-(tetrahydrofuran-2-ylmethyl)piperidin-4-yl]-1,2,4-oxadiazol-3-yl}-1H-indazole,
(16) 3-ethyl-6-fluoro-1-{5-[1-(tetrahydro-2H-pyran-4-ylmethyl)piperidin-4-yl]-1,2,4-oxadiazol-3-yl}-1H-indazole,
(17) 3-ethyl-6-fluoro-1-(5-{1-[2-(tetrahydro-2H-pyran-4-yl)ethyl]piperidin-4-yl}-1,2,4-oxadiazol-3-yl)-1H-indazole,
(18) 3-ethyl-6-fluoro-1-{5-[1-(tetrahydrofuran-3-yl)piperidin-4-yl]-1,2,4-oxadiazol-3-yl}-1H-indazole,
(19) 3-ethyl-6-fluoro-1-{5-[1-(propan-2-yl)piperidin-4-yl]-1,2,4-oxadiazol-3-yl}-1H-indazole,
(20) methyl 4-({4-[3-(3-ethyl-6-fluoro-1H-indazol-1-yl)-1,2,4-oxadiazol-5-yl]piperidin-1-yl}methyl)piperidine-1-carboxylate,
(21) methyl (2S)-2-({4-[3-(3-ethyl-1H-indazol-1-yl)-1,2,4-oxadiazol-5-yl]piperidin-1-yl}methyl)pyrrolidine-1-carboxylate,
(22) 2-fluoroethyl (2S)-2-({4-[3-(3-ethyl-7-fluoro-1H-indazol-1-yl)-1,2,4-oxadiazol-5-yl]piperidin-1-yl}methyl)pyrrolidine-1-carboxylate,
(23) 2-fluoroethyl (3S)-3-({4-[3-(3-ethyl-1H-indazol-1-yl)-1,2,4-oxadiazol-5-yl]piperidin-1-yl}methyl)pyrrolidine-1-carboxylate,
(24) 1-[3-({4-[3-(3-ethyl-7-fluoro-1H-indazol-1-yl)-1,2,4-oxadiazol-5-yl]piperidin-1-yl}methyl)azetidin-1-yl]-2-methoxyethanone,
(25) 1-{4-[3-(3-ethyl-6-fluoro-1H-indazol-1-yl)-1,2,4-oxadiazol-5-yl]-1,4′-bipiperidin-1′-yl}ethanone,
(26) 1-{4-[3-(3-ethyl-7-fluoro-1H-indazol-1-yl)-1,2,4-oxadiazol-5-yl]-1,4′-bipiperidin-1′-yl}ethanone,
(27) methyl 4-[3-(3-ethyl-6-fluoro-1H-indazol-1-yl)-1,2,4-oxadiazol-5-yl]-1,4′-bipiperidine-1′-carboxylate,
(28) 1-(4-{3-[7-fluoro-3-(propan-2-yl)-1H-indazol-1-yl]-1,2,4-oxadiazol-5-yl}-1,4′-bipiperidin-1′-yl)ethanone,
(29) 1-(4-{3-[7-fluoro-3-(propan-2-yl)-1H-indazol-1-yl]-1,2,4-oxadiazol-5-yl}-1,4′-bipiperidin-1′-yl)-2-hydroxyethanone,
(30) methyl 4-{3-[3-(3-ethyl-1,4-indazol-1-yl)-1,2,4-oxadiazol-5-yl]azetidin-1-yl}piperidine-1-carboxylate,
(31) 3-{4-[3-(3-ethyl-1H-indazol-1-yl)-1,2,4-oxadiazol-5-yl]piperidin-1-yl}propan-1-ol,
(32) cis-N-ethyl-3-[3-(3-ethyl-6-fluoro-1H-indazol-1-yl)-1,2,4-oxadiazol-5-yl]cyclobutanamine,
(33) 1-[(3R)-3-({4-[3-(3-ethyl-7-fluoro-1H-indazol-1-yl)-1,2,4-oxadiazol-5-yl]piperidin-1-yl}methyl)pyrrolidin-1-yl]ethanone,
(34) 1-[(3R)-3-({4-[3-(3-ethyl-7-fluoro-1H-indazol-1-yl)-1,2,4-oxadiazol-5-yl]piperidin-1-yl}methyl)pyrrolidin-1-yl]-2-methoxyethanone,
(35) 1-[(3R)-3-({4-[3-(3-ethyl-7-fluoro-1H-indazol-1-yl)-1,2,4-oxadiazol-5-yl]piperidin-1-yl}methyl)pyrrolidin-1-yl]-2-hydroxyethanone,
(36) 1-{4-[3-(3-ethyl-7-fluoro-1H-indazol-1-yl)-1,2,4-oxadiazol-5-yl]-1,4′-bipiperidin-1H-yl}-2-hydroxyethanone,

(37) 1-{4-[3-(3-ethyl-7-fluoro-1H-indazol-1-yl)-1,2,4-oxadiazol-5-yl]-1,4′-bipiperidin-1′-yl}-2-methoxyethanone,
(38) 4-[3-(3-ethyl-7-fluoro-1H-indazol-1-yl)-1,2,4-oxadiazol-5-yl]-1′-(methylsulfonyl)-1,4′-bipiperidine,
(39) 1-(4-{3-[7-fluoro-3-(propan-2-yl)-1H-indazol-1-yl]-1,2,4-oxadiazol-5-yl}-1,4′-bipiperidin-1′-yl)-2-methoxyethanone,
(40) 1-[(3S)-3-({4-[3-(3-ethyl-7-fluoro-1H-indazol-1-yl)-1,2,4-oxadiazol-5-yl]piperidin-1-yl}methyl)pyrrolidin-1-yl]ethanone,
(41) 1-[(3S)-3-({4-[3-(3-ethyl-7-fluoro-1H-indazol-1-yl)-1,2,4-oxadiazol-5-yl]piperidin-1-yl}methyl)pyrrolidin-1-yl]-2-methoxyethanone,
(42) 3-ethyl-7-fluoro-1-[5-(1-{[(3S)-1-(methylsulfonyl)pyrrolidin-3-yl]methyl}piperidin-4-yl)-1,2,4-oxadiazol-3-yl]-1,4-indazole,
(43) 3-ethyl-7-fluoro-1-[5-(1-{[(3R)-1-(methylsulfonyl)pyrrolidin-3-yl]methyl}piperidin-4-yl)-1,2,4-oxadiazol-3-yl]-1H-indazole,
(44) 1-[4-({4-[3-(3-ethyl-7-fluoro-1H-indazol-1-yl)-1,2,4-oxadiazol-5-yl]piperidin-1-yl}methyl)piperidin-1-yl]-2-hydroxyethanone,
(45) 1-[3-({4-[3-(3-ethyl-7-fluoro-1H-indazol-1-yl)-1,2,4-oxadiazol-5-yl]piperidin-1-yl}methyl)azetidin-1-yl]-2-hydroxyethanone,
(46) 1-{3-[(4-{3-[7-fluoro-3-(propan-2-yl)-1H-indazol-1-yl]-1,2,4-oxadiazol-5-yl}piperidin-1-yl)methyl]azetidin-1-yl}-2-methoxyethanone,
(47) 1-{3-[(4-{3-[7-fluoro-3-(propan-2-yl)-1H-indazol-1-yl]-1,2,4-oxadiazol-5-yl}piperidin-1-yl)methyl]azetidin-1-yl}ethanone,
(48) methyl 3-[(4-{3-[7-fluoro-3-(propan-2-yl)-1H-indazol-1-yl]-1,2,4-oxadiazol-5-yl}piperidin-1-yl)methyl]azetidine-1-carboxylate,
(49) 1-[3-({4-[3-(3-ethyl-7-fluoro-1H-indazol-1-yl)-1,2,4-oxadiazol-5-yl]piperidin-1-yl}methyl)azetidin-1-yl]ethanone
(50) 1-{(2R)-2-[(4-{3-[7-fluoro-3-(propan-2-yl)-1H-indazol-1-yl]-1,2,4-oxadiazol-5-yl}piperidin-1-yl)methyl]pyrrolidin-1-yl}-2-hydroxyethanone,
(51) 1-(4-{3-[7-fluoro-3-(propan-2-yl)-1H-indazol-1-yl]-1,2,4-oxadiazol-5-yl}-3′-methyl-1,4′-bipiperidin-1′-yl)-2-hydroxyethanone,
(52) 1-(3-{[(3R)-3-({3-[7-fluoro-3-(propan-2-yl)-1H-indazol-1-yl]-1,2,4-oxadiazol-5-yl}methyl)pyrrolidin-1-yl]methyl}azetidin-1-yl)ethanone,
(53) 1-(3-{[(3R)-3-({3-[7-fluoro-3-(propan-2-yl)-1H-indazol-1-yl]-1,2,4-oxadiazol-5-yl}methyl)pyrrolidin-1-yl]methyl}azetidin-1-yl)-2-hydroxyethanone,
(54) 1-[(3S)-3-{[(3R)-3-({3-[7-fluoro-3-(propan-2-yl)-1H-indazol-1-yl]-1,2,4-oxadiazol-5-yl}methyl)pyrrolidin-1-yl]methyl}pyrrolidin-1-yl]ethanone,
(55) 1-[(3S)-3-{[(3R)-3-({3-[7-fluoro-3-(propan-2-yl)-1H-indazol-1-yl]-1,2,4-oxadiazol-5-yl}methyl)pyrrolidin-1-yl]methyl}pyrrolidin-1-yl]-2-hydroxyethanone,
(56) 1-[(3R)-3-{[(3R)-3-({3-[7-fluoro-3-(propan-2-yl)-1H-indazol-1-yl]-1,2,4-oxadiazol-5-yl}methyl)pyrrolidin-1-yl]methyl}pyrrolidin-1-yl]-2-hydroxyethanone,
(57) 1-[(2S)-2-{[(3S)-3-({3-[7-fluoro-3-(propan-2-yl)-1H-indazol-1-yl]-1,2,4-oxadiazol-5-yl}methyl)pyrrolidin-1-yl]methyl}pyrrolidin-1-yl]-2-hydroxyethanone,
(58) 1-[(2R)-2-{[(3S)-3-({3-[7-fluoro-3-(propan-2-yl)-1,4-indazol-1-yl]-1,2,4-oxadiazol-5-yl}methyl)pyrrolidin-1-yl]methyl}pyrrolidin-1-yl]-2-hydroxyethanone,
(59) 1-[(3S)-3-{[(3S)-3-({3-[7-fluoro-3-(propan-2-yl)-1H-indazol-1-yl]-1,2,4-oxadiazol-5-yl}methyl)pyrrolidin-1-yl]methyl}pyrrolidin-1-yl]-2-hydroxyethanone,
(60) 1-[(3R)-3-{[(3S)-3-({3-[7-fluoro-3-(propan-2-yl)-1H-indazol-1-yl]-1,2,4-oxadiazol-5-yl}methyl)pyrrolidin-1-yl]methyl}pyrrolidin-1-yl]-2-hydroxyethanone,
(61) 1-{4-[3-(3-ethyl-7-fluoro-1H-indazol-1-yl)-1,2,4-oxadiazol-5-yl]-4′-methyl-1,4′-bipiperidin-1′-yl}-2-hydroxyethanon,
(62) 1-{4-[3-(3-ethyl-7-fluoro-1H-indazol-1-yl)-1,2,4-oxadiazol-5-yl]-4′-methyl-1,4′-bipiperidin-1′-yl}-2-methoxyethanone,
(63) (2S)-1-{4-[3-(3-ethyl-7-fluoro-1H-indazol-1-yl)-1,2,4-oxadiazol-5-yl]-4′-methyl-1,4′-bipiperidin-1′-yl}-2-hydroxypropan-1-one,
(64) 1-[(3S)-3-({4-[3-(3-ethyl-7-fluoro-1,4-indazol-1-yl)-1,2,4-oxadiazol-5-yl]piperidin-1-yl}methyl)pyrrolidin-1-yl]-2-hydroxyethanone,
(65) 1-[(2S)-2-({4-[3-(3-ethyl-7-fluoro-1H-indazol-1-yl)-1,2,4-oxadiazol-5-yl]piperidin-1-yl}methyl)pyrrolidin-1-yl]-2-hydroxyethanone,
(66) 1-{4-[(3S)-3-{[3-(3-ethyl-7-fluoro-1H-indazol-1-yl)-1,2,4-oxadiazol-5-yl]methyl}pyrrolidin-1-yl]piperidin-1-yl}ethanone,
(67) 1-{4-[(3R)-3-({3-[7-fluoro-3-(propan-2-yl)-1H-indazol-1-yl]-1,2,4-oxadiazol-5-yl}methyl)pyrrolidin-1-yl]piperidin-1-yl}-2-methoxyethanone,
(68) 1-(3-{[(3R)-3-({3-[7-fluoro-3-(propan-2-yl)-1H-indazol-1-yl]-1,2,4-oxadiazol-5-yl}methyl)pyrrolidin-1-yl]methyl}azetidin-1-yl)-2-methoxyethanone,
(69) 1-[(3S)-3-{[(3R)-3-({3-[7-fluoro-3-(propan-2-yl)-1H-indazol-1-yl]-1,2,4-oxadiazol-5-yl}methyl)pyrrolidin-1-yl]methyl}pyrrolidin-1-yl]-2-methoxyethanone,
(70) 1-[(3R)-3-{[(3R)-3-({3-[7-fluoro-3-(propan-2-yl)-1H-indazol-1-yl]-1,2,4-oxadiazol-5-yl}methyl)pyrrolidin-1-yl]methyl}pyrrolidin-1-yl]-2-methoxyethanone,
(71) 1-{4-[(3S)-3-({3-[7-fluoro-3-(propan-2-yl)-1H-indazol-1-yl]-1,2,4-oxadiazol-5-yl}methyl)pyrrolidin-1-yl]piperidin-1-yl}-2-methoxyethanone,
(72) 1-(3-{[(3S)-3-({3-[7-fluoro-3-(propan-2-yl)-1H-indazol-1-yl]-1,2,4-oxadiazol-5-yl}methyl)pyrrolidin-1-yl]methyl}azetidin-1-yl)-2-methoxyethanone,
(73) 1-[(3S)-3-{[(3S)-3-({3-[7-fluoro-3-(propan-2-yl)-1H-indazol-1-yl]-1,2,4-oxadiazol-5-yl}methyl)pyrrolidin-1-yl]methyl}pyrrolidin-1-yl]-2-methoxyethanone, and
(74) 1-(4-{3-[7-fluoro-3-(propan-2-yl)-1H-indazol-1-yl]-1,2,4-oxadiazol-5-yl}-3′-methyl-1,4′-bipiperidin-1′-yl)ethanone.

### Term 19

A pharmaceutical composition comprising the compound of any one of Terms 1 to 18 or a pharmaceutically acceptable salt thereof.

### Term 20

A serotonin-4 receptor agonist comprising the compound of any one of Terms 1 to 18 or a pharmaceutically acceptable salt thereof as an active ingredient.

### Term 21

A medicament for treating Alzheimer-type dementia comprising the compound of any one of Terms 1 to 18 or a pharmaceutically acceptable salt thereof as an active ingredient.

### Term 22

A method for treating a disease associated with serotonin-4 receptor comprising administering a therapeutically effective amount of the compound of any one of Terms 1 to 18 or a pharmaceutically acceptable salt thereof to a patient in need thereof.

### Term 23

A method for treating Alzheimer-type dementia comprising administering a therapeutically effective amount of the compound of any one of Terms 1 to 18 or a pharmaceutically acceptable salt thereof to a patient in need thereof.

### Effects of Invention

The present invention can provide compounds which act as an agonist or a partial agonist to a serotonin-4 receptor (hereinafter, optionally referred to as a 5-HT₄ receptor), and thus can provide a medicament for treating or preventing diseases or symptoms associated with serotonin-4 receptor. The diseases or symptoms suggested to be associated with serotonin-4 receptor include the following (i) to (v):

(i) neuropsychiatric diseases such as Alzheimer-type dementia, Lewy body dementia, vascular dementia, depression, posttraumatic stress disorder (PTSD), memory impairment, anxiety, and schizophrenia;
(ii) digestive system diseases such as irritable bowel syndrome, atonic constipation, habitual constipation, chronic constipation, constipation induced by drugs (e.g. morphine and antipsychotic drugs), constipation associated with Parkinson's disease, constipation associated with multiple sclerosis, constipation associated with diabetes mellitus, and constipation or dyschezia caused by contrast materials taken as a pretreatment for endoscopic examinations or barium enema X-ray examinations;
(iii) digestive system diseases such as functional dyspepsia, acute/chronic gastritis, reflux esophagitis, gastric ulcer, duodenal ulcer, gastric neurosis, postoperative paralytic ileus, senile ileus, non-erosive reflux disease, NSAID ulcer, diabetic gastroparesis, postgastrectomy syndrome, and intestinal pseudo-obstruction;
(iv) digestive system symptoms such as the digestive system diseases mentioned in the above (ii) and (iii), scleroderma, diabetes mellitus, anorexia in esophageal/biliary-tract diseases, nausea, emesis, bloating, epigastric discomfort, abdominal pain, heartburn, and belching; and
(v) urinary system diseases associated with dysuria such as urinary tract obstruction and prostatic hyperplasia.

The present compound is useful as a medicament for treating or preventing especially the neuropsychiatric diseases such as Alzheimer-type dementia mentioned in the above (i) because the compound shows an excellent 5-HT₄ receptor agonist activity and brain penetration.

### DESCRIPTION OF EMBODIMENTS

Hereinafter, the present invention is explained in more detail.

The “optionally substituted” or “substituted” group defined herein means that, unless otherwise indicated, the number of substituents is unlimited as long as possible, i.e. one or more substituents. Furthermore, unless otherwise noted, the definitions for each group may be also applied to a part of other groups or a substituent of other groups.

The terms used herein are set forth as below.

The “C₁₋₆ alkyl group” used herein includes a straight- or branched-chain alkyl group having 1 to 6 carbon atoms; and specifically methyl group, ethyl group, propyl group, isopropyl group, butyl group, isobutyl group, sec-butyl group, tert-butyl group, pentyl group, hexyl group, etc. The O₁₋₆ alkyl group includes preferably C₁₋₄ alkyl group; and specifically methyl group, ethyl group, propyl group, isopropyl group, butyl group, isobutyl group, sec-butyl group, and tert-butyl group.

The “C₂₋₆ alkenyl group” used herein includes a straight- or branched-chain alkenyl group having 2 to 6 carbon atoms and 1 to 2 double bonds. The C₂₋₆ alkenyl group includes specifically ethenyl group, 1-propenyl group, 1-methylvinyl group, 2-propenyl group, 1-butenyl group, 2-butenyl group, 3-butenyl group, 2-methyl-1-propenyl group, 2-methyl-2-propenyl group, 1-pentenyl group, 2-pentenyl group, 3-pentenyl group, 4-pentenyl group, 2-methyl-1-butenyl group, 2-methyl-2-butenyl group, 2-methyl-3-butenyl group, 1-hexenyl group, 2-hexenyl group, 3-hexenyl group, 4-hexenyl group, 5-hexenyl group, 2-methyl-1-pentenyl group, 2-propyl-2-propenyl group, 1-ethyl-2-methyl-2-propenyl group, 1-methyl-3-methyl-3-butenyl group, 4-methyl-4-pentenyl group, 1,3-butadienyl group, 1,5-hexadienyl group, etc.; and preferably ethenyl group, 1-propenyl group, 1-methylvinyl group, 2-propenyl group, 1-butenyl group, 2-butenyl group, 3-butenyl group, 2-methyl-1-propenyl group, and 2-methyl-2-propenyl group.

The “C₂₋₆ alkynyl group” used herein includes a straight- or branched-chain alkynyl group having 2 to 6 carbon atoms and 1 to 2 triple bonds, and more preferably 1 triple bond. The C₂₋₆ alkynyl group includes specifically ethynyl group, 1-propynyl group, 2-propynyl group, 1-butynyl group, 1-methyl-2-propynyl group, 3-butynyl group, 2-butynyl group, 1-pentynyl group, 1-ethyl-2-propynyl group, 4-pentynyl group, 3-pentynyl group, 2-pentynyl group, 1-methyl-2-butynyl group, 1-hexynyl group, 2-hexynyl group, 3-hexynyl group, 4-hexynyl group, 5-hexynyl group, etc; and preferably ethynyl group, 1-propynyl group, 2-propynyl group, 1-butynyl group, 1-methyl-2-propynyl group, 3-butynyl group, 2-butynyl group, 1-pentynyl group, 1-ethyl-2-propynyl group, 4-pentynyl group, 3-pentynyl group, 2-pentynyl group, and 1-methyl-2-butynyl group.

The “C₁₋₆ alkoxy group” used herein includes a straight- or branched-chain alkoxy group having 1 to 6 carbon atoms. The C₁₋₆ alkoxy group includes specifically methoxy group, ethoxy group, propoxy group, isopropoxy group, butoxy group, isobutoxy group, sec-butoxy group, tert-butoxy group, pentyloxy group, hexyloxy group etc.; and preferably methoxy group, ethoxy group, propoxy group, isopropoxy group, butoxy group, isobutoxy group, sec-butoxy group, and tert-butoxy group.

The “halogen atom” used herein includes fluorine atom, chlorine atom, bromine atom and iodine atom; preferably fluorine atom and chlorine atom; and more preferably fluorine atom.

The “C₃₋₆ alkenyl group” used herein includes a straight- or branched-chain alkenyl group having 3 to 6 carbon atoms and 1 to 2 double bonds. The C₃₋₆ alkenyl group includes specifically 1-propenyl group, 1-methylvinyl group, 2-propenyl group, 1-butenyl group, 2-butenyl group, 3-butenyl group, 2-methyl-1-propenyl group, 2-methyl-2-propenyl group, 1-pentenyl group, 2-pentenyl group, 3-pentenyl group, 4-pentenyl group, 2-methyl-1-butenyl group, 2-methyl-2-butenyl group, 2-methyl-3-butenyl group, 1-hexenyl group, 2-hexenyl group, 3-hexenyl group, 4-hexenyl group, 5-hexenyl group, 2-methyl-1-pentenyl group, 2-propyl-2-propenyl group, 1-ethyl-2-methyl-2-propenyl group, 1-methyl-3-methyl-3-butenyl group, 4-methyl-4-pentenyl group, 1,3-butadienyl group, 1,5-hexadienyl group, etc.; and preferably 1-propenyl group, 1-methylvinyl group, 2-propenyl group, 1-butenyl group, 2-butenyl group, 3-butenyl group, 2-methyl-1-propenyl group, 2-methyl-2-propenyl group, 1-pentenyl group, 2-pentenyl group, 3-pentenyl group, 4-pentenyl group, 2-methyl-1-butenyl group, 2-methyl-2-butenyl group, and 2-methyl-3-butenyl group.

The “C₃₋₆ alkynyl group” used herein includes a straight- or branched-chain alkynyl group having 3 to 6 carbon atoms and 1 to 2 triple bonds, and more preferably 1 triple bond. The C₃₋₆ alkynyl group includes specifically 1-propynyl group, 2-propynyl group, 1-butynyl group, 1-methyl-2-propynyl group, 3-butynyl group, 2-butynyl group, 1-pentynyl group, 1-ethyl-2-propynyl group, 4-pentynyl group, 3-pentynyl group, 2-pentynyl group, 1-methyl-2-butynyl group, 1-hexynyl group, 2-hexynyl group, 3-hexynyl group, 4-hexynyl group, 5-hexynyl group, etc; and preferably 1-propynyl group, 2-propynyl group, 1-butynyl group, 1-methyl-2-propynyl group, 3-butynyl group, 2-butynyl group, 1-pentynyl group, 1-ethyl-2-propynyl group, 4-pentynyl group, 3-pentynyl group, 2-pentynyl group, and 1-methyl-2-butynyl group.

The “C₃₋₈ cycloalkyl group” used herein includes a 3- to 8-membered cycloalkyl group; specifically cyclopropyl group, cyclobutyl group, cyclopentyl group, cyclohexyl group, cycloheptyl group, cyclooctyl group, etc.; and preferably cyclopropyl group, cyclobutyl group, cyclopentyl group, and cyclohexyl group.

The “C₅₋₈ cycloalkenyl group” used herein includes a 5- to 8-membered cycloalkenyl group; specifically cyclopentenyl group, 3-cyclopentenyl group, 4-cyclopentenyl group, 1-cyclohexenyl group, 3-cyclohexenyl group, 4-cyclohexenyl group, 1-cycloheptenyl group, 3-cycloheptenyl group, 4-cycloheptenyl group, 5-cycloheptenyl group, 1-cyclooctenyl group, 3-cyclooctenyl group, 4-cyclooctenyl group, 5-cyclooctenyl group, etc.; and preferably 1-cyclopentenyl group, 3-cyclopentenyl group, 4-cyclopentenyl group, 1-cyclohexenyl group, 3-cyclohexenyl group, and 4-cyclohexenyl group.

The “aryl group” used herein includes a 6- to 10-membered monocyclic or bicyclic aryl group; and specifically phenyl group, 1-naphthyl group, 2-naphthyl group, etc.

The “heteroaryl group” used herein includes a 5- to 10-membered monocyclic or bicyclic heteroaryl group comprising 1 to 4 heteroatoms selected from the group consisting of 1 to 3 nitrogen atoms, 1 oxygen atom and 1 sulfur atom. The monocyclic heteroaryl group includes specifically pyrrolyl group, imidazolyl group, triazolyl group, tetrazolyl group, furyl group, thienyl group, oxazolyl group, thiazolyl group, pyridyl group, pyrimidinyl group, pyrazinyl group, pyridazinyl group, triazinyl group, etc.; and preferably pyrrolyl group, imidazolyl group, triazolyl group, tetrazolyl group, furyl group, thienyl group, oxazolyl group, pyridyl group, pyrimidinyl group, pyrazinyl group, and pyridazinyl group.

The bicyclic heteroaryl group includes indolyl group, benzofuryl group, benzothienyl group, quinolinyl group, benzisoxazolyl group, etc. The binding site of the heteroaryl group is not limited and may be any carbon atom or nitrogen atom therein as long as the bond is chemically stable. The heteroaryl group includes preferably indolyl group and quinolinyl group.

The “5- to 9-membered monocyclic or 7- to 10-membered bicyclic non-aromatic unsaturated heterocyclic group” used herein includes a 5- to 9-membered monocyclic or 7- to 10-membered bicyclic non-aromatic unsaturated heterocyclic group comprising 1 to 4 heteroatoms selected from the group consisting of 1 to 3 nitrogen atoms, 1 oxygen atom and 1 sulfur atom. The monocyclic non-aromatic unsaturated heterocyclic group includes a 5-membered non-aromatic unsaturated heterocyclic group having 1 double bond and a 6- or 7-membered non-aromatic unsaturated heterocyclic group having 1 or 2 double bonds; and specifically pyrrolinyl group, 2,5-dihydrofuryl group, etc.

The bicyclic non-aromatic unsaturated heterocyclic group includes a 7- to 10-membered non-aromatic unsaturated heterocyclic group which can be obtained by replacing one or more double bonds of the bicyclic heteroaryl group with single bonds; and specifically 2,3-dihydrobenzofuryl group, 2,3-dihydrobenzothienyl group, etc.

The binding site of the non-aromatic unsaturated heterocyclic group is not limited and may be any carbon atom or nitrogen atom therein as long as the bond is chemically stable.

The “4- to 9-membered monocyclic or 7- to 10-membered bicyclic saturated heterocyclic group” used herein includes a 4- to 9-membered monocyclic or 7- to 10-membered bicyclic saturated heterocyclic group comprising 1 to 4 heteroatoms selected from the group consisting of 1 to 4 nitrogen atoms, 1 oxygen atom and 1 sulfur atom. The monocyclic saturated heterocyclic group includes specifically azetidinyl group, pyrrolidinyl group, tetrahydrofuryl group, tetrahydrothienyl group, piperazinyl group, piperidinyl group, morpholinyl group, thiomorpholinyl group, tetrahydropyranyl group, hexahydroazepinyl group, 1,4-hexahydrooxazepinyl group, 1,4-hexahydrodiazepinyl group, etc.; and preferably azetidinyl group, pyrrolidinyl group, tetrahydrofuryl group, piperazinyl group, piperidinyl group, morpholinyl group, and tetrahydropyranyl group. The bicyclic saturated heterocyclic group includes a 7- to 10-membered saturated heterocyclic group; and specifically quinuclidinyl group, etc.

Any carbon atom in the saturated heterocyclic group may be substituted with oxo group. The saturated heterocyclic group substituted with oxo group includes specifically 2-oxopyrrolidinyl group, 2-oxotetrahydrofuryl group, etc.

The binding site of the saturated heterocyclic group is not limited and may be any carbon atom or nitrogen atom therein as long as the bond is chemically stable.

The “C₁₋₄ alkyl group” used herein includes a straight- or branched-chain alkyl group having 1 to 4 carbon atoms; specifically methyl group, ethyl group, propyl group, isopropyl group, butyl group, isobutyl group, sec-butyl group, tert-butyl group etc.; and preferably methyl group, ethyl group, propyl group, and isopropyl group.

The “C₁₋₄ alkoxy group” used herein includes a straight- or branched-chain alkoxy group having 1 to 4 carbon atoms; specifically methoxy group, ethoxy group, propoxy group, isopropoxy group, butoxy group, isobutoxy group, sec-butoxy group, tert-butoxy group etc.; and preferably methoxy group, ethoxy group, propoxy group, isopropoxy group, and tert-butoxy group.

The “C₁₋₄ haloalkoxy group” used herein includes an alkoxy group having 1 to 4 carbon atoms which is substituted with the same or a different 1 to 5 halogen atoms; specifically fluoromethoxy group, difluoromethoxy group, trifluoromethoxy group, pentafluoroethoxy group, 2-fluoroethoxy group, 2,2-difluoroethoxy group, etc.; and preferably trifluoromethoxy group and pentafluoroethoxy group.

The “C₁₋₄ haloalkyl group” used herein includes an alkyl group having 1 to 4 carbon atoms which is substituted with the same or a different 1 to 5 halogen atoms; specifically fluoromethyl group, difluoromethyl group, trifluoromethyl group, 2-fluoroethyl group, 2,2-difluoroethyl group, 4-fluoro butyl group, etc.; and preferably fluoromethyl group, difluoromethyl group, and trifluoromethyl group.

The “aryloxy group” used herein includes an aryloxy group having 6 to 10 carbon atoms; and specifically phenoxy group, naphthoxy group etc. straight- or branched-chain alkanoyl group having 2 to 6 carbon atoms; specifically acetyl group, propanoyl group, butanoyl group, 2-methylpropanoyl group, pentanoyl group, 3-methylbutanoyl group, 2-methylbutanoyl group, hexanoyl group, etc.; and preferably acetyl group, propanoyl group, butanoyl group, and 2-methylpropanoyl group.

The “optionally-substituted amino group” used herein includes, for example, amino, mono- or di-substituted amino, and 4- to 7-membered cyclic amino. The substituents of the “mono- or di-substituted amino” includes, for example, “C₁₋₆ alkyl”, “C₃₋₇ cycloalkyl”, “C₃₋₇ cycloalkyl C₁₋₄ alkyl”, etc.

The “monosubstituted amino” includes, for example, “mono C₁₋₆ alkylamino” such as methylamino, ethylamino, propylamino, 1-methylethylamino, butylamino, 2-methylpropylamino, 1-methylpropylamino, and 1,1-dimethylethylamino; “C₃₋₇ cycloalkyl amino” such as cyclopropylamino, cyclobutylamino, cyclopentylamino, cyclohexylamino, and cycloheptylamino; and “(C₃₋₇ cycloalkyl C₁₋₄ alkyl)amino” such as cyclopropylmethyl-amino, cyclobutylmethylamino, cyclopentylmethylamino, cyclohexylmethylamino, and cycloheptylmethylamino.

The “di-substituted amino” includes, for example, “di-C₁₋₆ alkylamino” such as dimethylamino, diethylamino, dipropylamino, di-1-methylethylamino, dibutylamino, di-2-methylpropylamino, di-1-methylpropylamino, and di-1,1-dimethylethylamino; and “N”-(C₁₋₆ alkyl)-N—(C₃₋₇ cycloalkyl)amino” such as methylcyclopropylamino, methyl-cyclobutylamino, methylcyclopentylamino, methylcyclo-hexylamino, and methylcycloheptylamino.

The “4- to 7-membered cyclic amino group” includes, for example, a 4- to 7-membered monocyclic amino group comprising additional 0 to 2 heteroatoms independently-selected from the group consisting of nitrogen atom, oxygen atom and sulfur atom; and the binding site thereof is the nitrogen atom in the ring. The optionally-substituted amino group includes, for example, azetidino, pyrrolidino, piperazino, piperidino, morpholino, thiomorpholino, azepano, and oxoazepano; preferably amino, methylamino, ethylamino, cyclopropylamino, cyclobutylamino, dimethylamino, di-1-methylethylamino, methylcyclopropylamino, azetidino, pyrrolidino, piperazino, piperidino, and morpholino; and more preferably amino, methylamino, dimethylamino, azetidino, pyrrolidino, and piperidino.

The “saturated or unsaturated 4- to 9-membered monocyclic or 7- to 10-membered bicyclic nitrogen-containing heterocyclic group comprising the adjacent nitrogen atom and additional 0 to 2 heteroatoms independently-selected from the group consisting of 1 to 2 nitrogen atoms, 1 oxygen atom and 1 sulfur atom” used herein includes specifically azetidinyl group, pyrrolidinyl group, piperazinyl group, piperidinyl group, morpholinyl group, thiomorpholinyl group, hexahydroazepinyl group, 1,4-hexahydrooxazepinyl group, 1,4-hexahydrodiazepinyl group, indolinyl group, isoindolinyl group, 1,2,3,4-tetrahydroquinolinyl group, 1,2,3,4-tetrahydroisoquinolinyl group, 1,2,3,4-tetrahydroquinoxalinyl group, 3,4-dihydrobenzo-1,4-oxadinyl group, 3,4-dihydrobenzo-1,4-thiadinyl group, 3-azabicyclo[3.2.0]heptanyl group, octahydroisoindolyl group, octahydroindolyl group, decahydroquinolinyl group, decahydroisoquinolinyl group, decahydroquinoxalinyl group, octahydrobenzo-1,4-oxadinyl group, octahydrobenzo-1,4-thiadinyl group, etc.; preferably azetidinyl group, pyrrolidinyl group, piperazinyl group, piperidinyl group, morpholinyl group, hexahydroazepinyl group, 1,4-hexahydrooxazepinyl group, indolinyl group, isoindolinyl group, 1,2,3,4-tetrahydroquinolinyl group, 1,2,3,4-tetrahydroisoquinolinyl group, and 3,4-dihydrobenzo-1,4-oxadinyl group; and more preferably pyrrolidinyl group, piperazinyl group, piperidinyl group, and morpholinyl group.

The “saturated or unsaturated 3- to 8-membered ring that may comprise 1 oxygen atom” used herein includes specifically cyclopropane ring, cyclobutane ring, cyclopentane ring, cyclohexane ring, cycloheptane ring, cyclooctane ring, oxetane ring, tetrahydrofuran ring, tetrahydropyran ring, oxepane ring, benzene ring, etc.; and preferably cyclopropane ring, cyclobutane ring, cyclopentane ring, and cyclohexane ring.

The “bicyclic or a spiro compound in which the above-mentioned ring is attached with the pair of R¹⁰ and R¹¹, or R¹⁰′ and R¹¹′” used herein includes specifically indoline, isoindoline, 1,2,3,4-tetrahydroquinoline, 1,2,3,4-tetra-hydroisoquinoline, 3-azabicyclo[3.2.0]heptane, 7-azabicyclo[2.2.1]heptane, 6-azabicyclo[3.1.1]heptane, 2-azabicyclo[2.2.1]heptane, 3-azabicyclo[3.1.1]heptane, 8-azabicyclo[3.2.1]octane, 2-azabicyclo[2.2.2]octane, 3-azabicyclo[3.2.1]octane, octahydroisoindone, octahydroindoline, decahydroquinoline, decahydroisoquinoline, octahydrocyclopenta[b]pyrrole, octahydrocyclopenta[c]pyrrole, 2-oxa-7-azaspiro[3.5]nonane, 2-oxa-8-azaspiro[4.5]decane, etc.; preferably indoline, isoindoline, 1,2,3,4-tetrahydroquinoline, 1,2,3,4-tetrahydroisoquinoline, 3-azabicyclo-[3.2.0]heptane, 7-azabicyclo[2.2.1]heptane, 6-azabicyclo-[3.1.1]heptane, 2-azabicyclo[2.2.1]heptane, 3-azabicyclo-[3.1.1]heptane, 8-azabicyclo[3.2.1]octane, and 2-azabicyclo[2.2.2]octane, 3-azabicyclo[3.2.1]octane; and more preferably 7-azabicyclo[2.2.1]heptane, 8-azabicyclo[3.2.1]-octane, and 3-azabicyclo[3.2.1]octane.

The “C₃₋₈ monocyclic, C₇₋₁₀ bicyclic or C₇₋₁₂ tricyclic cycloalkyl group” used herein includes 3- to 8-membered monocyclic cycloalkyl group, 7- to 10-membered bicyclic cycloalkyl group, or 7- to 12-membered tricyclic cycloalkyl group, respectively.

The monocyclic cycloalkyl group used herein includes specifically cyclopropyl group, cyclobutyl group, cyclopentyl group, cyclohexyl group, cycloheptyl group, cyclooctyl group, etc.; and preferably cyclopropyl group, cyclobutyl group, cyclopentyl group, and cyclohexyl group.

The bicyclic cycloalkyl group used herein includes specifically octahydropentalenyl group, octahydro-1H-indenyl group, bicyclo[2.2.1]heptyl group, bicyclo[2.2.2]octyl group, bicyclo[4.2.0]octyl group, decahydronaphthalenyl group, etc.; and preferably bicyclo[2.2.1]heptyl group and bicyclo[2.2.2]octyl group.

The tricyclic cycloalkyl group used herein includes specifically adamantyl group, etc.

The “C₅₋₈ monocyclic or C₇₋₁₀ bicyclic cycloalkenyl group” used herein includes 5- to 8-membered monocyclic cycloalkenyl group or 7- to 10-membered bicyclic cycloalkenyl group, respectively.

The monocyclic cycloalkenyl group used herein includes specifically 1-cyclopentenyl group, 3-cyclopentenyl group, 4-cyclopentenyl group, 1-cyclohexenyl group, 3-cyclohexenyl group, 4-cyclohexenyl group, 1-cycloheptenyl group, 3-cycloheptenyl group, 4-cycloheptenyl group, 5-cycloheptenyl group, 1-cyclooctenyl group, 3-cyclooctenyl group, 4-cyclooctenyl group, 5-cyclooctenyl group, etc; preferably 1-cyclopentenyl group, 3-cyclopentenyl group, 4-cyclopentenyl group, 1-cyclohexenyl group, 3-cyclohexenyl group, and 4-cyclohexenyl group.

The bicyclic cycloalkenyl group used herein includes specifically bicyclo[2.2.1]hept-2-enyl group, bicyclo-[2.2.2]oct-2-enyl group, etc.

Regarding the “saturated or unsaturated 4- to 8-membered monocyclic nitrogen-containing heterocyclic group comprising the adjacent nitrogen atom and additional 0 to 2 heteroatoms independently-selected from the group consisting of 1 to 2 nitrogen atoms, 1 oxygen atom and 1 sulfur atom” used herein, the saturated monocyclic nitrogen-containing heterocyclic group includes specifically azetidinyl group, pyrrolidinyl group, piperazinyl group, piperidinyl group, morpholinyl group, thiomorpholinyl group, hexahydroazepinyl group, 1,4-hexahydrooxazepinyl group, etc.; and preferably azetidinyl group, pyrrolidinyl group, piperazinyl group, piperidinyl group, and morpholinyl group.

The unsaturated monocyclic nitrogen-containing heterocyclic group includes specifically pyrrolyl group, imidazolyl group, triazolyl group, tetrazolyl group, 1,2,3,6-tetrahydropyridyl group, 2,5-dihydro-1H-pyrrolyl group, etc.

The “C₁₋₆ alkoxycarbonyl group” used herein includes a carbonyl group having a straight- or branched-chain alkoxy group having 1 to 6 carbon atoms; specifically methoxycarbonyl group, ethoxycarbonyl group, propoxycarbonyl group, isopropoxycarbonyl group, butoxycarbonyl group, isobutoxycarbonyl group, sec-butoxycarbonyl group, tert-butoxycarbonyl group, pentyloxycarbonyl group, hexyloxycarbonyl group etc.; and preferably methoxycarbonyl group, ethoxycarbonyl group, propoxycarbonyl group, isopropoxycarbonyl group, butoxycarbonyl group, isobutoxycarbonyl group, sec-butoxycarbonyl group, and tert-butoxycarbonyl group.

The “C₁₋₆ alkylsulfonyl group” used herein includes a straight- or branched-chain alkylsulfonyl group having 1 to 6 carbon atoms; specifically methylsulfonyl group, ethylsulfonyl group, propylsulfonyl group, isopropyl sulfonyl group, butylsulfonyl group, isobutylsulfonyl group, sec-butylsulfonyl group, tert-butylsulfonyl group, pentylsulfonyl group, hexylsulfonyl group, etc.; and preferably methylsulfonyl group, ethylsulfonyl group, propylsulfonyl group, isopropyl sulfonyl group, butylsulfonyl group, isobutylsulfonyl group, sec-butylsulfonyl group, and tert-butylsulfonyl group.

The “saturated or unsaturated 6- to 9-membered ring optionally comprising 1 oxygen atom formed by taking R³ and R⁴ together” used herein includes specifically the 6- to 9-membered ring of the following Formulae (E-1) to (E-16):

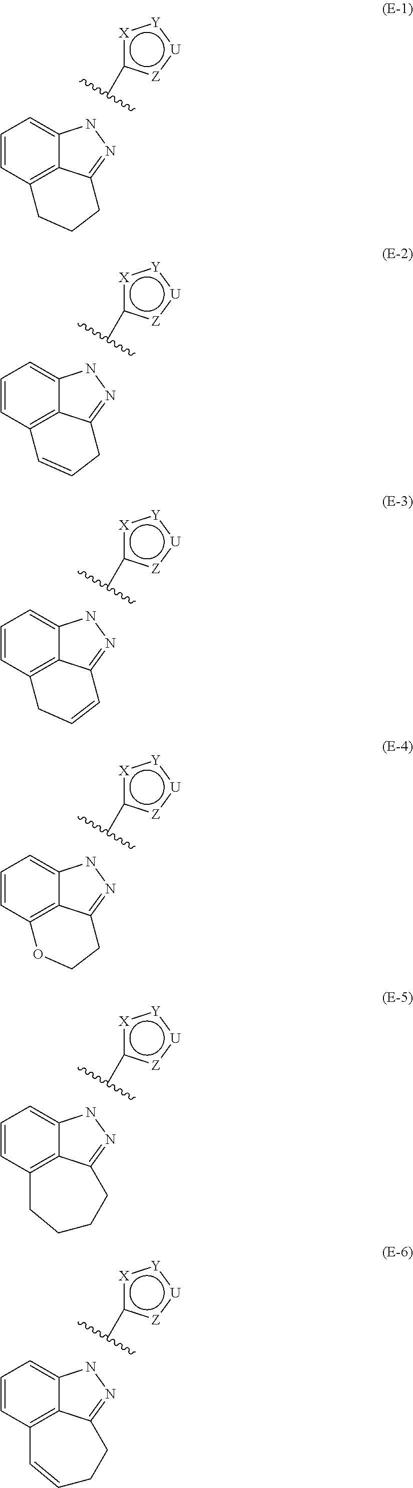

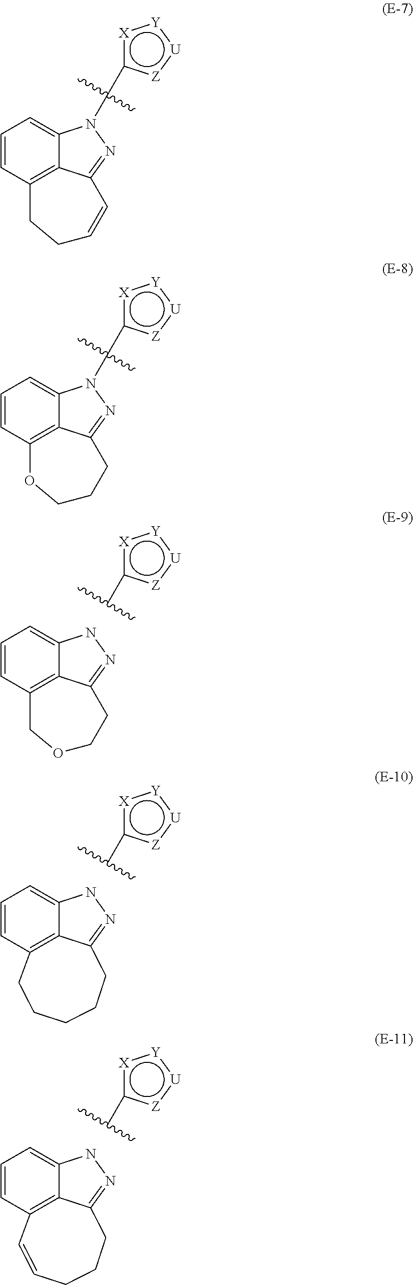

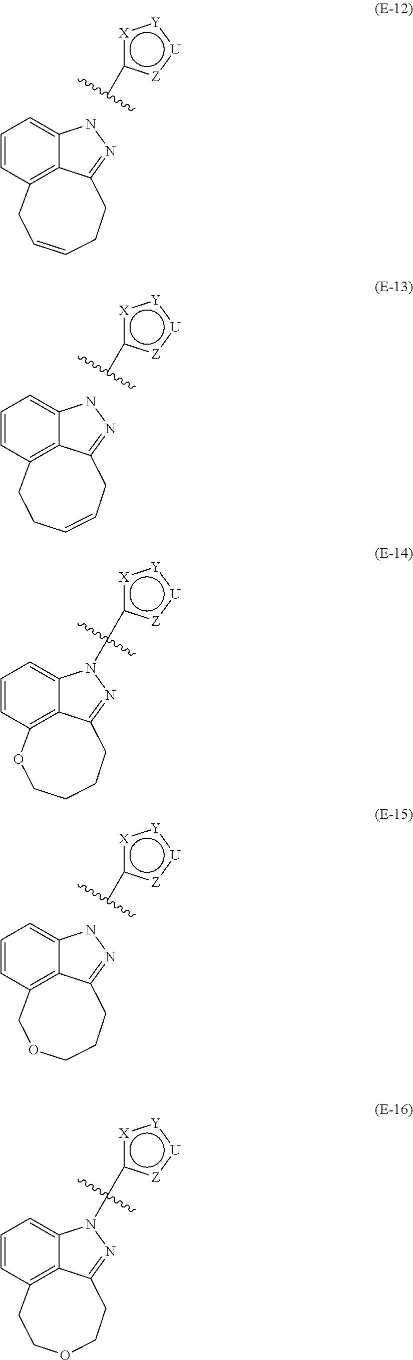

and the like; preferably Formulae (E-1), (E-4), (E-5), (E-8), (E-9), (E-10), and (E-14).

The “5-membered heteroaryl which is the substructure of Formula (1), i.e. the following Formula (F):

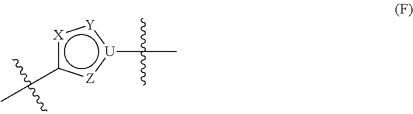

wherein U is carbon atom or nitrogen atom; and X, Y and Z are independently selected from the group consisting of oxygen atom, nitrogen atom, sulfur atom and carbon atom, provided that at least one of X, Y and Z is oxygen atom, sulfur atom, or nitrogen atom” includes heteroaryl of the following Formulae (F-1) to (F-16):

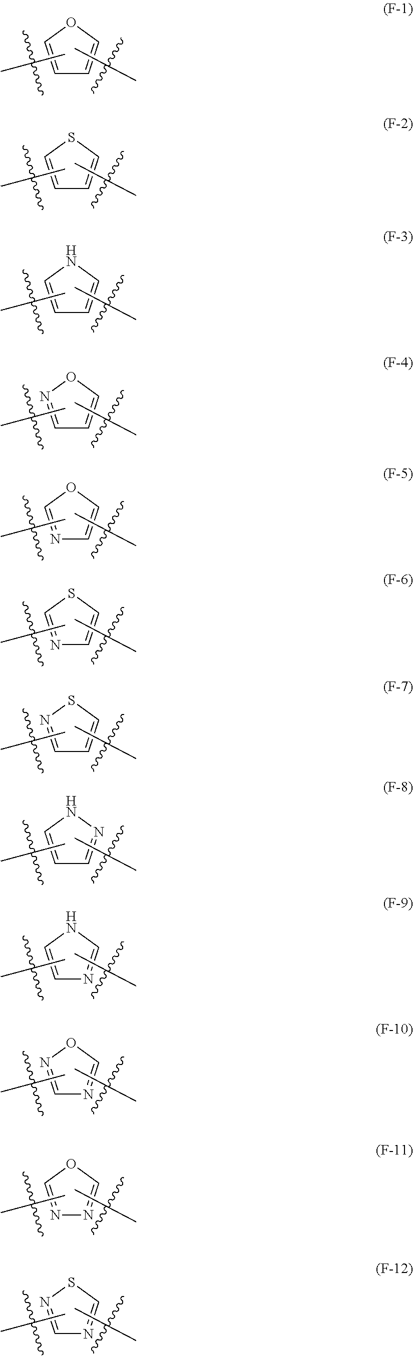

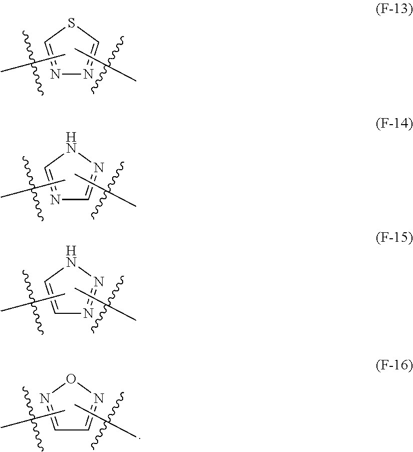

The binding site of the heteroaryl is not limited and may be any carbon atom or nitrogen atom therein as long as the bond is chemically stable. The heteroaryl includes preferably Formulae (F-10) to (F-13), and more preferably Formulae (F-10) to (F-11).

Hereinafter, each group of the present invention is explained.

The “A” used herein includes preferably Formula (A-1) and Formula (A-3), and more preferably Formula (A-1).

The “B” used herein includes preferably Formula (B-1) and Formula (B-2), and more preferably Formula (B-2).

The “R⁸, R⁹ and D” used herein independently include preferably hydrogen atom, an optionally-substituted C₁₋₆ alkyl group, an optionally-substituted C₃₋₈ monocyclic, C₇₋₁₀ bicyclic or C₇₋₁₂ tricyclic cycloalkyl group, and —(CH₂)ᵤ—R¹².

The “R¹³” used herein includes preferably Formula (R¹²-1), Formula (R¹²-3), and Formula (R¹²-5).

The “R¹³” used herein includes preferably hydrogen atom, an optionally-substituted C₁₋₆ alkyl group, an optionally-substituted C₃₋₈ cycloalkyl group, —COR¹⁶, SO₂R¹⁶, COOR¹⁶, and —CONR¹⁹R²⁰; more preferably an optionally-substituted C₁₋₆ alkyl group, an optionally-substituted C₃₋₈ cycloalkyl group, —COR¹⁶, —SO₂R¹⁶, and —COOR¹⁶; and even more preferably —COR¹⁶, —SO₂R¹⁶, and —COOR¹⁶.

The “R¹⁶” used herein includes preferably an optionally-substituted C₁₋₆ alkyl group, an optionally-substituted C₃₋₈ cycloalkyl group, an optionally-substituted aryl group, and an optionally-substituted heteroaryl group; and more preferably an optionally-substituted C₁₋₆ alkyl group and an optionally-substituted C₃₋₈ cycloalkyl group.

The “R¹⁴ and R¹⁵” used herein independently include preferably hydrogen atom, an optionally-substituted C₁₋₆ alkyl group, an optionally-substituted C₃₋₈ cycloalkyl group, an optionally-substituted aryl group, and an optionally-substituted heteroaryl group; and more preferably an optionally-substituted C₁₋₆ alkyl group and an optionally-substituted C₃₋₈ cycloalkyl group.

The “R¹” used herein includes preferably hydrogen atom, halogen atom, an optionally-substituted C₁₋₆ alkyl group, and an optionally-substituted C₃₋₈ cycloalkyl group; and more preferably hydrogen atom.

The “R²” used herein includes preferably hydrogen atom, halogen atom, hydroxy group, an optionally-substituted C₁₋₆ alkyl group, an optionally-substituted C₃₋₈ cycloalkyl group, an optionally-substituted C₁₋₆ alkoxy group, an optionally-substituted haloalkyl group, an optionally-substituted C₁₋₄ haloalkoxy group, cyano group, nitro group, an optionally-substituted aryl group, an optionally-substituted heteroaryl group, and an optionally-substituted amino group; more preferably hydrogen atom, halogen atom, an optionally-substituted C₁₋₆ alkyl group, an optionally-substituted C₁₋₆ alkoxy group, an optionally-substituted C₁₋₄ haloalkyl group, and an optionally-substituted C₁₋₄ haloalkoxy group; and even more preferably hydrogen atom, halogen atom, and an optionally-substituted C₁₋₆ alkyl group.

The “R³” used herein includes preferably hydrogen atom, halogen atom, an optionally-substituted C₁₋₆ alkyl group, and an optionally-substituted C₃₋₈ cycloalkyl group; and more preferably hydrogen atom, halogen atom, and an optionally-substituted C₁₋₆ alkyl group.

The “R⁴” used herein includes preferably hydrogen atom, halogen atom, hydroxy group, an optionally-substituted C₁₋₆ alkyl group, an optionally-substituted C₃₋₈ cycloalkyl group, an optionally-substituted C₁₋₆ alkoxy group, an optionally-substituted C₁₋₄ haloalkyl group, an optionally-substituted C₁₋₄ haloalkoxy group, cyano group, nitro group, an optionally-substituted aryl group, an optionally-substituted heteroaryl group, and an optionally-substituted amino group; more preferably hydrogen atom, halogen atom, an optionally-substituted C₁₋₆ alkyl group, an optionally-substituted C₁₋₆ alkoxy group, an optionally-substituted C₁₋₄ haloalkyl group, and an optionally-substituted C₁₋₄ haloalkoxy group; and even more preferably hydrogen atom, halogen atom, and an optionally-substituted C₁₋₆ alkyl group.

The “R⁵” used herein includes preferably hydrogen atom, halogen atom, hydroxy group, an optionally-substituted C₁₋₆ alkyl group, an optionally-substituted O₃₋₈ cycloalkyl group, an optionally-substituted C₁₋₆ alkoxy group, an optionally-substituted C₁₋₄ haloalkyl group, an optionally-substituted C₁₋₄ haloalkoxy group, cyano group, nitro group, an optionally-substituted aryl group, an optionally-substituted heteroaryl group, and an optionally-substituted amino group; more preferably hydrogen atom, halogen atom, an optionally-substituted C₁₋₆ alkyl group, an optionally-substituted C₁₋₆ alkoxy group, an optionally-substituted C₁₋₄ haloalkyl group, and an optionally-substituted C₁₋₄ haloalkoxy group; and even more preferably hydrogen atom, halogen atom, and an optionally-substituted C₁₋₆ alkyl group.

The “R⁶” used herein includes preferably hydrogen atom, halogen atom, hydroxy group, an optionally-substituted C₁₋₆ alkyl group, an optionally-substituted C₃₋₈ cycloalkyl group, an optionally-substituted C₁₋₆ alkoxy group, an optionally-substituted C₁₋₄ haloalkyl group, an optionally-substituted C₁₋₄ haloalkoxy group, cyano group, nitro group, an optionally-substituted aryl group, an optionally-substituted heteroaryl group, and an optionally-substituted amino group; more preferably hydrogen atom, halogen atom, an optionally-substituted C₁₋₆ alkyl group, an optionally-substituted C₁₋₆ alkoxy group, an optionally-substituted C₁₋₄ haloalkyl group, and an optionally-substituted C₁₋₄ haloalkoxy group; and even more preferably hydrogen atom, halogen atom, and an optionally-substituted C₁₋₆ alkyl group.

The “R⁸′ and R⁹′” used herein independently include preferably hydrogen atom, an optionally-substituted C₁₋₆ alkyl group, an optionally-substituted C₃₋₈ cycloalkyl group, an optionally-substituted C₅₋₈ cycloalkenyl group, an optionally-substituted aryl group, and an optionally-substituted heteroaryl group; and more preferably an optionally-substituted C₁₋₆ alkyl group and an optionally-substituted C₃₋₈ cycloalkyl group.

The “R¹⁰, R¹⁰′, R¹¹ and R¹¹′” used herein independently include preferably hydrogen atom, halogen atom, hydroxy group, an optionally-substituted C₁₋₆ alkyl group, and an optionally-substituted C₁₋₆ alkoxy group; and more preferably hydrogen atom, an optionally-substituted C₁₋₆ alkyl group, and an optionally-substituted C₁₋₆ alkoxy group.

The “l” used herein includes an integer of preferably 0 and 1.

The “m” used herein includes an integer of preferably 0 and 1.

The “n” used herein includes an integer of preferably 0 and 1.

The “o” used herein includes an integer of preferably 0 and 1.

The “q” used herein includes an integer of preferably 1 to 3.

The “r and r′” used herein independently include an integer of preferably 1 to 2.

The “s and s′” used herein independently include an integer of preferably 0 and 1.

The “t and t′” used herein independently include an integer of preferably 1.

The “u” used herein includes an integer of preferably 0 to 2, and more preferably 0 and 1.

The “v” used herein includes an integer of preferably 1 and 2.

The “Formulae (A-1) to (A-4)” used herein may be independently and optionally substituted with one or more substituents independently-selected from the group consisting of preferably C₁₋₆ alkyl group, hydroxy group, and C₁₋₆ alkoxy group at each substitutable position thereof.

In case that R⁸, R⁹ and D are independently C₁₋₆ alkyl group, C₃₋₆ alkenyl group, C₃₋₆ alkynyl group, C₃₋₈ monocyclic, C₇₋₁₀ bicyclic or C₇₋₁₂ tricyclic cycloalkyl group, or C₅₋₈ monocyclic or C₇₋₁₀ bicyclic cycloalkenyl group; the R⁸, R⁹ and D may be independently and optionally substituted with one or more substituents independently-selected from the group consisting of preferably alkyl group, hydroxy group, C₁₋₄ alkoxy group, C₁₋₄ haloalkyl group, and aryl group at each substitutable position thereof.

In case that R⁸, R⁹ and D are independently —(CH₂)ᵤ—R¹² wherein u is an integer of 1 to 4; the alkylene chain may be optionally substituted with one or more substituents independently-selected from the group consisting of preferably C₁₋₆ alkyl group, hydroxy group, and C₁₋₆ alkoxy group at each substitutable position thereof.

In case that R¹³ is C₁₋₆ alkyl group, C₃₋₆ alkenyl group, C₃₋₆ alkynyl group, C₃₋₈ cycloalkyl group, or C₅₋₈ cycloalkenyl group; the R¹³ may be independently and optionally substituted with one or more substituents independently-selected from the group consisting of preferably C₁₋₄ alkyl group, hydroxy group, C₁₋₄ alkoxy group, C₁₋₄ haloalkyl group, C₁₋₄ haloalkoxy group, and halogen atom at each substitutable position thereof.

In case that R¹⁶ is C₁₋₆ alkyl group, C₃₋₆ alkenyl group, C₃₋₆ alkynyl group, C₃₋₈ cycloalkyl group, C₅₋₈ cycloalkenyl group, 5- to 9-membered monocyclic or 7- to 10-membered bicyclic non-aromatic unsaturated heterocyclic group, or 4- to 9-membered monocyclic or 7- to 10-membered bicyclic saturated heterocyclic group; the R¹⁶ may be independently and optionally substituted with one or more substituents independently-selected from the group consisting of preferably C₁₋₄ alkyl group, hydroxy group, C₁₋₄ alkoxy group, C₁₋₄ haloalkyl group, C₁₋₄ haloalkoxy group, oxo group, aryl group, heteroaryl group, and halogen atom; and more preferably C₁₋₄ alkyl group, hydroxy group, C₁₋₄ alkoxy group, C₁₋₄ haloalkyl group, and C₁₋₄ haloalkoxy group at each substitutable position thereof.

In case that R¹⁶ is aryl group or heteroaryl group; the R¹⁶ may be independently and optionally substituted with one or more substituents independently-selected from the group consisting of preferably halogen atom, hydroxy group, C₁₋₄ alkyl group, C₁₋₄ alkoxy group, C₁₋₄ haloalkyl group, C₁₋₄ haloalkoxy group, cyano group, and an optionally-substituted amino group; more preferably, halogen atom, C₁₋₄ alkyl group, C₁₋₄ alkoxy group, C₁₋₄ haloalkyl group, C₁₋₄ haloalkoxy group, and an optionally-substituted amino group; and even more preferably halogen atom, C₁₋₄ alkyl group, C₁₋₄ alkoxy group, and an optionally-substituted amino group at each substitutable position thereof.

In case that R¹⁹ and R²⁰ are taken together with the adjacent nitrogen atom to form a saturated or unsaturated 4- to 8-membered monocyclic nitrogen-containing heterocyclic group comprising additional 0 to 2 heteroatoms independently-selected from the group consisting of 1 to 2 nitrogen atoms, 1 oxygen atom and 1 sulfur atom; the formed ring may be optionally substituted with one or more substituents independently-selected from the group consisting of preferably C₁₋₄ alkyl group, hydroxy group, alkoxy group, oxo group and halogen atom at each substitutable position thereof.

In case that R¹⁴ and R¹⁵ are independently C₁₋₆ alkyl group, C₃₋₆ alkenyl group, C₃₋₆ alkynyl group, C₃₋₈ cycloalkyl group, C₅₋₈ cycloalkenyl group, 5- to 9-membered monocyclic or 7- to 10-membered bicyclic non-aromatic unsaturated heterocyclic group, 4- to 9-membered monocyclic or 7- to 10-membered bicyclic saturated heterocyclic group, C₂₋₆ alkanoyl group, C₁₋₆ alkoxycarbonyl group, or C₁₋₆ alkylsulfonyl group; the R¹⁴ and R¹⁵ may be independently and optionally substituted with one or more substituents independently-selected from the group consisting of preferably C₁₋₄ alkyl group, hydroxy group, C₁₋₄ alkoxy group, oxo group, aryl group, heteroaryl group, and halogen atom; and more preferably C₁₋₄ alkyl group, hydroxy group, C₁₋₄ alkoxy group, and halogen atom at each substitutable position thereof.

In case that R¹⁴ and R¹⁵ are taken together with the adjacent nitrogen atom to form a saturated or unsaturated 4- to 9-membered monocyclic or 7- to 10-membered bicyclic nitrogen-containing heterocyclic group comprising additional 0 to 2 heteroatoms independently-selected from the group consisting of 1 to 2 nitrogen atoms, 1 oxygen atom and 1 sulfur atom; the formed ring may be optionally substituted with one or more substituents independently-selected from the group consisting of preferably C₁₋₄ alkyl group, hydroxy group, C₁₋₄ alkoxy group, C₁₋₄ haloalkyl group, C₁₋₄ haloalkoxy group, oxo group, and halogen atom; and more preferably, C₁₋₄ alkyl group, hydroxy group, C₁₋₄ alkoxy group, oxo group, and halogen atom at each substitutable position thereof.

In case that R⁸′ and R⁹′ are independently C₁₋₆ alkyl group, C₃₋₆ alkenyl group, C₃₋₆ alkynyl group, C₃₋₆ cycloalkyl group, C₅₋₆ cycloalkenyl group, 5- to 9-membered monocyclic or 7- to 10-membered bicyclic non-aromatic unsaturated heterocyclic group, or 4- to 9-membered monocyclic or 7- to 10-membered bicyclic saturated heterocyclic group; the R⁸′ and R⁹′ may be independently and optionally substituted with one or more substituents independently-selected from the group consisting of preferably C₁₋₄ alkyl group, hydroxy group, C₁₋₄ alkoxy group, C₁₋₄ haloalkoxy group, oxo group, aryl group, heteroaryl group, aryloxy group, and halogen atom; and more preferably C₁₋₄ alkyl group, hydroxy group, C₁₋₄ alkoxy group, oxo group, and halogen atom at each substitutable position thereof.

In case that a pair of R⁸ and R⁹, and a pair of R^{8′} and R^{9′} are independently taken together with the adjacent nitrogen atom to form a saturated or unsaturated 4- to 9-membered monocyclic or 7- to 10-membered bicyclic nitrogen-containing heterocyclic group comprising additional 0 to 2 heteroatoms independently-selected from the group consisting of 1 to 2 nitrogen atoms, 1 oxygen atom and 1 sulfur atom; the formed rings may be independently and optionally substituted with one or more substituents independently-selected from the group consisting of preferably C₁₋₄ alkyl group, and oxo group at each substitutable position thereof.

In case that R¹⁰, R¹⁰′, R¹¹ and R^{11′} are independently C₁₋₆ alkyl group, C₂₋₆ alkenyl group, C₂₋₆ alkynyl group, or C₁₋₆ alkoxy group; the R¹⁰, R¹⁰′, R¹¹ and R¹¹′ may be independently and optionally substituted with one or more substituents independently-selected from the group consisting of preferably C₁₋₄ alkyl group, hydroxy group, C₁₋₄ alkoxy group, C₁₋₄ haloalkoxy group, oxo group, aryl group, heteroaryl group, aryloxy group, and halogen atom; and more preferably, C₁₋₄ alkyl group, hydroxy group, C₁₋₄ alkoxy group, and halogen atom at each substitutable position thereof.

In case that a pair of R¹⁰ and R¹¹, and a pair of R¹⁰′ and R¹¹′ are independently taken together to form an optionally-substituted saturated or unsaturated 3- to 8-membered ring that may comprise 1 oxygen atom, which may be a bicyclic or a spiro compound with the ring to which the pair of R¹⁰ and R¹¹, or R¹⁰′ and R¹¹′ is attached; the formed rings may be independently and optionally substituted with one or more substituents independently-selected from the group consisting of preferably C₁₋₄ alkyl group, hydroxy group, C₁₋₄ alkoxy group, oxo group, and halogen atom at each substitutable position thereof.

In case that R¹ is C₁₋₆ alkyl group, C₂₋₆ alkenyl group, C₂₋₆ alkynyl group, C₃₋₈ cycloalkyl group, or C₅₋₈ cycloalkenyl group; the R¹ may be independently and optionally substituted with one or more substituents independently-selected from the group consisting of preferably C₁₋₄ alkyl group, hydroxy group, C₁₋₄ alkoxy group, C₁₋₄ haloalkyl group, C₁₋₄ haloalkoxy group, and halogen atom; and more preferably C₁₋₄ alkyl group, hydroxy group, and C₁₋₄ alkoxy group at each substitutable position thereof.

In case that R² is C₁₋₆ alkyl group, C₂₋₆ alkenyl group, C₂₋₆ alkynyl group, C₃₋₈ cycloalkyl group, C₅₋₈ cycloalkenyl group, C₁₋₆ alkoxy group, C₁₋₄ haloalkyl group, or C₁₋₄ haloalkoxy group; the R² may be independently and optionally substituted with one or more substituents independently-selected from the group consisting of preferably C₁₋₄ alkyl group, hydroxy group, C₁₋₄ alkoxy group, C₁₋₄ haloalkyl group, C₁₋₄ haloalkoxy group, and halogen atom; and more preferably C₁₋₄ alkyl group, hydroxy group, and C₁₋₄ alkoxy group at each substitutable position thereof.

In case that R³ is C₁₋₆ alkyl group, C₂₋₆ alkenyl group, C₂₋₆ alkynyl group, C₃₋₈ cycloalkyl group, C₅₋₈ cycloalkenyl group, C₁₋₆ alkoxy group, C₁₋₄ haloalkyl group, haloalkoxy group, 5- to 9-membered monocyclic or 7- to 10-membered bicyclic non-aromatic unsaturated heterocyclic group, or 4- to 9-membered monocyclic or 7- to 10-membered bicyclic saturated heterocyclic group; the R³ may be independently and optionally substituted with one or more substituents independently-selected from the group consisting of preferably C₁₋₄ alkyl group, hydroxy group, alkoxy group, C₁₋₄ haloalkyl group, C₁₋₄ haloalkoxy group, and halogen atom; and more preferably C₁₋₄ alkyl group, hydroxy group, and C₁₋₄ alkoxy group at each substitutable position thereof.

In case that R⁴ is C₁₋₆ alkyl group, C₂₋₆ alkenyl group, C₂₋₆ alkynyl group, C₃₋₈ cycloalkyl group, C₅₋₈ cycloalkenyl group, C₁₋₆ alkoxy group, C₁₋₄ haloalkyl group, or C₁₋₄ haloalkoxy group; the R⁴ may be independently and optionally substituted with one or more substituents independently-selected from the group consisting of preferably C₁₋₄ alkyl group, hydroxy group, C₁₋₄ alkoxy group, C₁₋₄ haloalkyl group, C₁₋₄ haloalkoxy group, and halogen atom; and more preferably C₁₋₄ alkyl group, hydroxy group, and C₁₋₄ alkoxy group at each substitutable position thereof.

In case that R³ and R⁴ are taken together to form a saturated or unsaturated 6- to 9-membered ring optionally comprising 1 oxygen atom; the formed ring may be optionally substituted with one or more substituents independently-selected from the group consisting of preferably C₁₋₄ alkyl group, hydroxy group, alkoxy group, oxo group, and halogen atom at each substitutable position thereof.

In case that R⁵ and R⁶ are independently C₁₋₆ alkyl group, C₂₋₆ alkenyl group, C₂₋₆ alkynyl group, C₃₋₈ cycloalkyl group, C₅₋₈ cycloalkenyl group, C₁₋₆ alkoxy group, C₁₋₄ haloalkyl group, or C₁₋₄ haloalkoxy group; the R⁵ and R⁶ may be independently and optionally substituted with one or more substituents independently-selected from the group consisting of preferably C₁₋₄ alkyl group, hydroxy group, alkoxy group, C₁₋₄ haloalkyl group, C₁₋₄ haloalkoxy group, and halogen atom; and more preferably C₁₋₄ alkyl group, hydroxy group, and C₁₋₄ alkoxy group at each substitutable position thereof.

Hereinafter, the compound of Formula (1) in the present invention is explained in more detail.

The compound of Formula (1) may encompass all tautomers, geometric isomers, stereoisomers and a mixture thereof depending on the types of substituents.

To be more specific, the compound of Formula (1) with one or more chiral carbon atoms exists in the form of a diastereomer or optical isomer, and the present invention encompasses a mixture or an isolated one of the diastereomer or optical isomer.

The present invention also includes an isotope-labeled compound of Formula (1) and a pharmaceutically acceptable salt thereof, wherein the isotope-labeled compound is the same as the compound of Formula (1) except that one or more atoms in the compound have an atomic mass or a mass number which is different from the typical atomic mass or mass number present in nature. The present compound includes an isotope of, for example, hydrogen, carbon, nitrogen, oxygen, phosphorus, fluorine, bromine, and chlorine. In specific, the present compound includes isotopes such as ²H, ³H, ¹¹C, ¹³C, ¹⁴C, ¹³N, ¹⁵N, ¹⁸O, ¹⁷O, ¹⁵O, ¹⁸F, ⁷⁵Br, ⁷⁶Br, ⁷⁷Br, ⁸² Br, and ³⁶Cl. The present invention also includes the present compounds which comprise the above-mentioned isotopes and/or other isotopes of other atoms, and pharmaceutically acceptable salts thereof.

A particular isotope-labeled compound of the present invention (e.g. a compound comprising radioisotopes such as ¹¹C, ³H and ¹⁸F) is useful, for example, in a tissue distribution assay of the medicament and/or substrate, and especially useful as a diagnostic agent to find out the localization of the 5-HT₄ receptor subtype which is a serotonin receptor. The isotopes of tritium (i. ³H), carbon-11 (i.e. ¹¹C), and ¹⁸F are especially preferable because they can be easily manufactured and detected. Thus, these compounds are also useful to assess the density of the said receptor in each region of the central nervous system, and to assess the receptor occupancy obtained by using a certain concentration of these compounds. The results of the assessment are likely to be helpful in determining the dosage and dose of these compounds. Furthermore, from this viewpoint, these isotope-labeled compounds can also be used for studying the characteristics of diseases which could have not been diagnosed in the past.

In addition, the substitution with heavy isotopes such as deuterium, i.e. ²H can provide some therapeutic benefits owing to increased metabolic stability (such as prolongation of in vivo half-life and decrease of the required dosage), and thus the compound having heavy isotopes may be preferable in some situations.

The pharmaceutically acceptable salt used herein includes an acid addition salt and a base addition salt. For example, the acid addition salt includes an inorganic acid salt such as hydrochloride, hydrobromide, sulfate, hydrogen sulfate, hydroiodide, nitrate, and phosphate; and an organic acid salt such as citrate, oxalate, acetate, formate, propionate, benzoate, trifluoroacetate, fumarate, maleate, malonate, succinate, tartrate, hydrogen tartrate, lactate, malate, pyruvate, gluconate, saccharate, methanesulfonate, ethanesulfonate, benzenesulfonate, p-toluenesulfonate, and pamoate [i.e. 1,1′-methylene-bis-(2-hydroxy-3-naphthoate)]. The base addition salt includes an inorganic base salt such as sodium salt, potassium salt, calcium salt, magnesium salt, and ammonium salt; and an organic base salt such as triethylammonium salt, triethanolammonium salt, pyridinium salt, and diisopropylammonium salt. The pharmaceutically acceptable salt may also include a basic amino acid salt such as alginate, aspartate, and glutamate; and an acidic amino acid salt. The salt used herein includes preferably hydrochloride, hydrobromide, sulfate, phosphate, citrate, fumarate, maleate, malonate, succinate, tartrate, lactate, malate, pyruvate, methanesulfonate, and benzenesulfonate.

The compound of Formula (1) and a pharmaceutically acceptable salt thereof may be a solvate such as a hydrate or an ethanolate, and the hydrate and/or solvate are also included in the present compound.

### Process of the Present Compound

Hereinafter, several processes of the present compound of Formula (1):

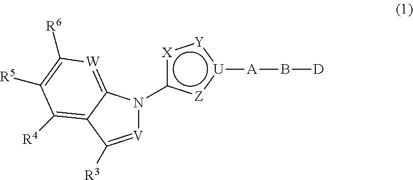

are explained with examples, but the present invention should not be limited thereto. The compound of Formula (1) can be synthesized from a well-known compound by combining several well-known processes. For example, the compound can be prepared as follows.

### (Process 1)

The compound of Formula (1) in which, for example, D is (CH₂)ᵤ—(R¹²-1) [i.e. Compound (1′)] can be prepared by the following process:

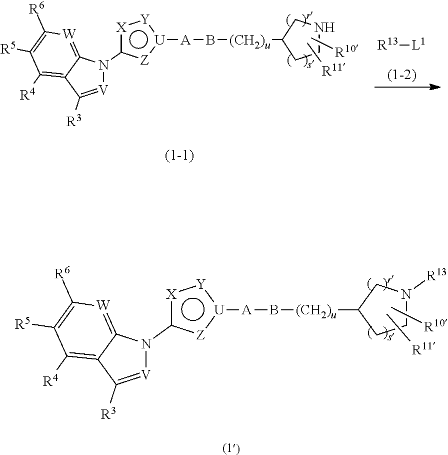

wherein r′, s′, u, A, B, U, V, W, X, Y, Z, R³, R⁴, R⁵, R⁶, R¹⁰, R¹¹′ and R¹³ are as defined above, and L¹ is a leaving group.

In specific, the compound of Formula (1′) can be prepared by reacting the compound of Formula (1-1) with the reactive derivative of Formula (1-2) in the presence of an appropriate additive such as a base.

In case that —R¹³ is —COR¹⁶ wherein R¹⁶ is as defined above, the reactive derivative of Formula (1-2) wherein L¹ is hydroxy group may include the carboxylic acid compound of Formula (1-3):

R¹⁶—COOH wherein R¹⁶ is as defined above, and an alkyl ester thereof (in particular, a methyl ester), an active ester thereof, an acid anhydride thereof, and a carboxylic halide thereof (in particular, a carboxylic chloride).

The carboxylic acid compound of Formula (1-3) may be reacted in the presence of a condensing agent such as 1,3-dicyclohexylcarbodiimide, 1-ethyl-3-(3-dimethylamino-propyl)carbodiimide hydrochloride, N,N′-carbonyldiimidazole, benzotriazol-1-yloxytris(dimethylamino)phosphonium hexa-fluorophosphate, N,N′-carbonyldisuccinimide, 1-ethoxycarbonyl-2-ethoxy-1,2-dihydroquinoline, diphenylphosphoryl azide, and propanephosphonic anhydride. In case that 1,3-dicyclohexylcarbodiimide or 1-ethyl-3-(3-dimethylamino-propyl)carbodiimide hydrochloride is used as the condensing agent, N-hydroxysuccinimide, 1-hydroxybenzotriazole, 3-hydroxy-1,2,3-benzotriazin-4(3H)-one, N-hydroxy-5-norbornene-2,3-dicarboxylmide, etc. may be added to the reaction.

The active ester of the carboxylic acid compound of Formula (1-3) specifically includes p-nitrophenyl ester, pentachlorophenyl ester, pentafluorophenyl ester, N-hydroxysuccinimide ester, N-hydroxyphthalimide ester, 1-hydroxybenzotriazole ester, 8-hydroxyquinoline ester, 2-hydroxyphenyl ester, etc.

The acid anhydride of carboxylic acid compound of Formula (1-3) used herein may include a symmetrical acid anhydride or a mixed acid anhydride; and the mixed acid anhydride specifically includes a mixed acid anhydride with an alkyl chlorocarbonate such as ethyl chlorocarbonate and isobutyl chlorocarbonate, a mixed acid anhydride with an aralkyl chlorocarbonate such as benzyl chlorocarbonate, a mixed acid anhydride with an aryl chlorocarbonate such as phenyl chlorocarbonate, and a mixed acid anhydride with an alkanoic acid such as isovaleric acid and pivalic acid.

In case that —R¹³ of Formula (1′) is —COOR¹⁶ wherein R¹⁶ is as defined above, the reactive derivative of Formula (1-2) may include the compound of Formula (1-4):

R¹⁶O—CO-L¹ wherein L¹ and R¹⁶ are defined as above. The compound of Formula (1-4) wherein L¹ is chlorine atom is commercially available, or can be prepared by reacting R¹⁶OH and phosgene, diphosgene or a phosgene equivalent such as triphosgene.

In case that —R¹³ of Formula (1′) is —SO₂—R¹⁶ wherein R¹⁶ is as defined above, the reactive derivative of Formula (1-2) may include the compound of Formula (1-5):

R¹⁶—SO₂-L¹ wherein L¹ and R¹⁶ are defined as above.

In case that —R¹³ of Formula (1′) is —CONR¹⁹R²⁰ wherein R¹⁹ and R²⁰ are as defined above, the reactive derivative of Formula (1-2) may include the compound of Formula (1-6):

R¹⁹R²⁰N—CO-L¹ wherein L¹, R¹⁹ and R²⁰ are defined as above.

The reaction of the compound of Formula (1-1) and the reactive derivative of Formula (1-2) can be carried out in the presence or absence of a solvent. The solvent used herein should be optionally selected depending on the types of starting compounds and other factors, and includes, for example, aromatic hydrocarbons such as benzene, toluene, and xylene; ethers such as diethyl ether, tetrahydrofuran, dioxane, cyclopentyl methyl ether; halogenated hydrocarbons such as methylene chloride and chloroform; ketones such as acetone and methyl ethyl ketone; ethyl acetate; acetonitrile; N,N-dimethylformamide; and dimethylsulfoxide. These solvents may be used alone or in a mixture of two or more.

The reaction may be optionally carried out in the presence of a base. The base used herein includes specifically alkali hydroxides such as sodium hydroxide and potassium hydroxide; alkaline carbonates such as sodium carbonate and potassium carbonate; alkaline bicarbonates such as sodium bicarbonate and potassium bicarbonate; and organic bases such as triethylamine, tributylamine, diisopropylethylamine, and N-methylmorpholine. In order to also use the compound of Formula (1-1) as a base, an excess amount of the compound may be used.

The reaction temperature depends on the types of the starting compound used herein or other factors; and it is typically about −30° C. to about 200° C., and preferably about −10° C. to about 150° C.

The leaving group of L¹ used herein includes, for example, halogen atoms such as chlorine, bromine, and iodine; alkylsulfonyloxy groups such as methanesulfonyloxy group; and arylsulfonyloxy groups such as benzenesulfonyloxy group and p-toluenesulfonyloxy group; and preferably halogen atoms (in particular, chlorine and bromine), methanesulfonyloxy, and p-toluenesulfonyloxy.

The compound of Formula (1-1) described in Process 1 in which, for example, B is (B-2), D is (CH₂)ᵤ—(R¹²-1), and u is 1 [i.e. Compound (1-1′)] can be prepared by the following Process 2.

Furthermore, in case that B is (B-2), D is (CH₂)ᵤ—(R¹²-1), and u is 0 [i.e. Compound (1-1″)], the compound can be prepared by the following Process 3.

### (Process 2)

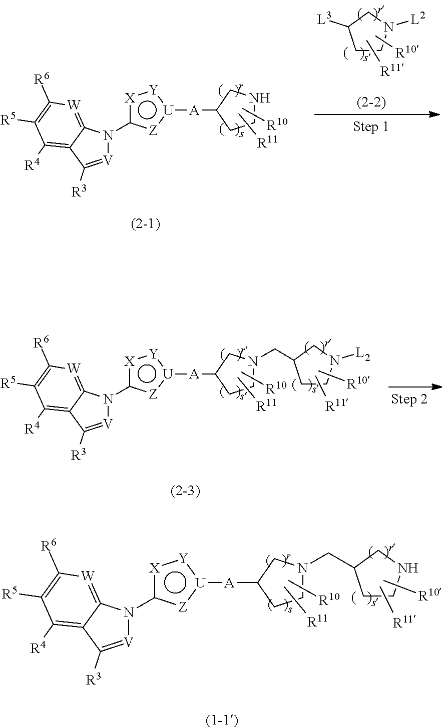

wherein

r, s, r′, s′, A, U, V, W, X, Y, Z, R³, R⁴, R⁵, R⁶, R¹⁶, R¹¹, R¹⁰′ and R¹¹′ are as defined above,

L² is a protecting group which may be eliminated by hydrolysis or hydrogenolysis, and

L³ is —CH₂-L⁴ (wherein L⁴ is a leaving group) or formyl group.

### (Process 3)

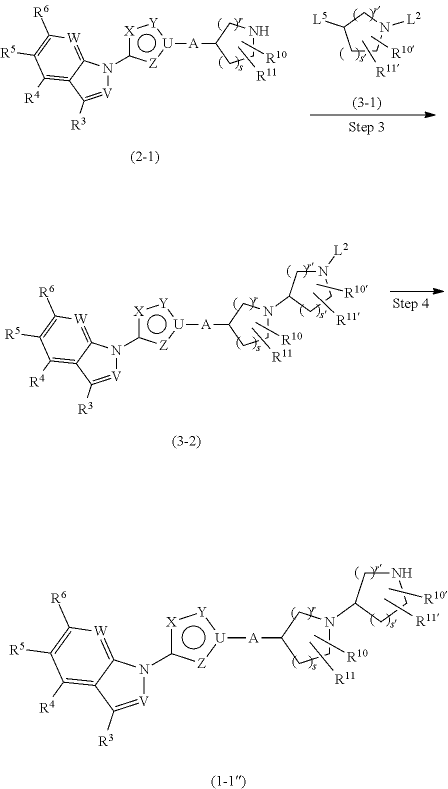

wherein

r, s, r′, s′, A, U, V, W, X, Y, Z, R³, R⁴, R⁵, R⁶, R¹⁰, R¹¹, R¹⁰′ and R¹¹′ are as defined above,

L² is a protecting group which may be eliminated by hydrolysis or hydrogenolysis, and

L⁵ is oxo group or a leaving group.

Hereinafter, Steps 1 to 4 of the above Processes 2 and 3 are explained.

1) Alkylation step by substitution reaction (Step 1, Step 3)

When L³ is —CH₂-L⁴ (wherein L⁴ is a leaving group) in the compound of Formula (2-2) which is an intermediate of Process 2 and when L⁵ is a leaving group in the compound of Formula (3-1) which is an intermediate of Process 3, Step 1 and Step 3 are an alkylation step carried out by a substitution reaction in the presence or absence of a solvent. The solvent used herein should be optionally selected depending on the types of starting compounds, etc., and includes for example, aromatic hydrocarbons such as benzene, toluene, and xylene; ethers such as diethyl ether, tetrahydrofuran, cyclopentyl methyl ether, and dioxane; halogenated hydrocarbons such as methylene chloride and chloroform; alcohols such as ethanol, isopropanol, and ethylene glycol; ketones such as acetone and methyl ethyl ketone; ethyl acetate, acetonitrile; N,N-dimethylformamide; and dimethylsulfoxide. These solvents may be used alone or in a mixture of two or more.

The reaction can be carried out in the presence of a base as appropriate, and the base used herein includes alkali hydroxides such as sodium hydroxide and potassium hydroxide; alkaline carbonates such as sodium carbonate and potassium carbonate; alkaline bicarbonates such as sodium bicarbonate and potassium bicarbonate; and organic bases such as triethylamine, tributylamine, diisopropylethylamine, and N-methylmorpholine. In order to also use the compound of Formula (2-1) as a base, an excess amount of the compound may be used.

The leaving groups of L⁴ and L⁵ include, for example, halogen atoms such as chlorine, bromine, and iodine; alkylsulfonyloxy groups such as methanesulfonyloxy group; and arylsulfonyloxy groups such as benzenesulfonyloxy group and p-toluenesulfonyloxy group; and preferably halogen atoms (in particular, chlorine and bromine), methanesulfonyloxy, and p-toluenesulfonyloxy. In case that L⁴ and L⁵ are chlorine or bromine, the reaction smoothly proceeds by adding alkali metal iodides such as sodium iodide and potassium iodide.

The reaction temperature depends on the types of the starting compound used herein or other factors; and it is typically about 0° C. to about 200° C., preferably about 20° C. to about 150° C.

The compounds of Formula (2-2) and Formula (3-1) are commercially available, or may be prepared according to known methods. In specific, the compounds of Formula (2-2) and Formula (3-1) wherein L⁴ and L⁵ are a leaving group can be prepared from the corresponding alcohol derivatives of Formula (2-2a) and Formula (3-1a) by converting the corresponding group into a leaving group according to conventional methods:

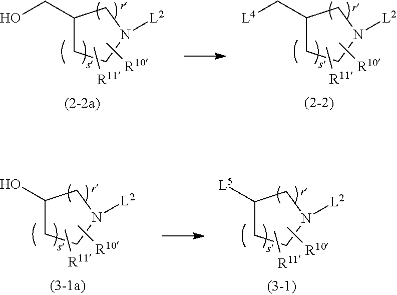

wherein r′, s′, R¹⁰′, R¹¹′ and L² are as defined above; and L⁴ and L⁵ are a leaving group.

For example, the compound of Formula (2-2a) can be reacted with carbon tetrachloride or carbon tetrabromide and triphenylphosphine to give a compound wherein L⁴ is chlorine atom or bromine atom. Alternatively, the compound of Formula (2-2a) can be reacted with sulfonyl chloride compounds such as benzenesulfonyl chloride in the presence of a base to give a compound wherein L⁴ is arylsulfonyloxy group or alkylsulfonyloxy group.

### 2) Reductive Alkylation Step (Step 1, Step 3)

When L³ is formyl group in the compound of Formula (2-2) which is an intermediate of Process 2 and when L⁵ is oxo group in the compound of Formula (3-1) which is an intermediate of Process 3, Step 1 and Step 3 are a reductive alkylation step and can be, for example, carried out under the following conditions:

1. a catalytic reduction using platinum oxide or palladium carbon as a catalyst in the presence of, if necessary, a catalytic amount of acid
2. a reduction using borane complex such as pyridine borane and triethylamine borane, sodium borohydride, sodium triacetoxyhydroborate, or sodium cyanoborohydride in the presence of, if necessary, a catalytic or excess amount of acid. The solvent used herein includes the solvents mentioned in the above-mentioned 1). The acid used herein includes, for example, p-toluenesulfonic acid, hydrogen chloride, and titanium tetraisopropoxide. The reaction temperature is usually about 0° C. to about 100° C., and preferably about 20° C. to about 80° C.

The compounds of Formula (2-2) and Formula (3-1) used herein are commercially available, or may be prepared according to known methods. In specific, the compounds of Formula (2-2) wherein L³ is formyl group and Formula (3-1) wherein L⁵ is oxo group can be prepared by oxidizing the corresponding alcohol derivatives of Formula (2-2) and Formula (3-1a) according to conventional methods. For example, the compounds of Formula (2-2a) and Formula (3-1a) can be oxidized with phosgene, dimethylsulfoxide and triethylamine.

Alternatively, the compound of Formula (2-2) can also be prepared by reducing the corresponding carboxylic acid or an ester thereof according to conventional methods, and for example, by reducing the compound of Formula (2-2b) with DIBAH (i.e. diisobutylaluminium hydride).

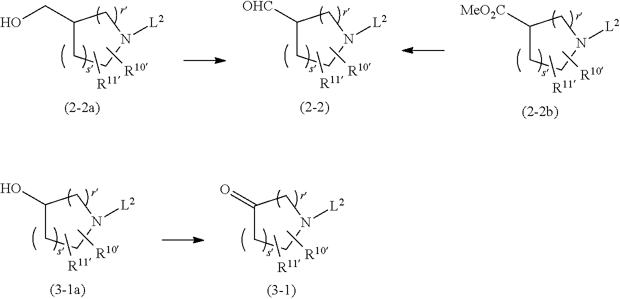

wherein r′, s′, R¹⁰′, R¹¹′ and L² are as defined above.

In addition, the compound of Formula (2-2b) used herein is commercially available, or may be prepared according to known methods.

### 3) Deprotection Step (Step 2, Step 4)

Step 2 and Step 4 are a deprotection reaction. Among the protecting groups of L² used in Processes 2 and 3, protecting groups which may be eliminated by hydrolysis include, for example, ethoxycarbonyl group, tert-butoxycarbonyl group, acetyl group, benzoyl group, trifluoroacetyl group, benzyloxycarbonyl group, 3- or 4-chlorobenzyloxycarbonyl group, triphenylmethyl group, methanesulfonyl group, and p-toluenesulfonyl group.

The deprotection by hydrolysis can be carried out according to conventional methods, and for example, it may be carried out by contacting the protecting group with water in a suitable solvent under an acidic or basic condition. The solvent used herein includes, for example, alcohols such as methanol, ethanol, and isopropanol; acetonitrile; dioxane; water; and a mixture thereof. The acid used herein specifically includes mineral acids such as hydrochloric acid, hydrobromic acid, hydroiodic acid, and sulfuric acid; and organic acids such as formic acid, acetic acid, trifluoroacetic acid, p-toluenesulfonic acid, and methanesulfonic acid. The base used herein specifically includes alkali hydroxides such as sodium hydroxide and potassium hydroxide; and alkaline carbonates such as sodium carbonate and potassium carbonate. The reaction temperature is usually about 0° C. to about 150° C.

Among the protecting groups of L², protecting groups which may be eliminated by hydrogenolysis include, for example, benzyloxycarbonyl group, 3- or 4-chlorobenzyloxycarbonyl group, benzyl group, and 4-methoxybenzyl group. The deprotection by hydrogenolysis can be carried out according to conventional methods, and for example, it may be carried out by reacting the protecting group in a suitable solvent in the presence of a catalyst (such as palladium carbon and Raney nickel), and in the presence of hydrogen or a hydrogen donor (such as ammonium formate and cyclohexene). The solvent used herein includes, for example, alcohols such as ethanol and methanol, water, acetic acid, dioxane, tetrahydrofuran, ethyl acetate, and N,N-dimethylformamide. The reaction is carried out at a temperature of usually about 0° C. to about 80° C., under normal or high pressure.

The compound of Formula (2-1) described in Processes 2 and 3 can be prepared by the methods of the following Processes 4 to 6.

### (Process 4)

The compound of Formula (2-1′) wherein, for example, X is nitrogen atom, Z is nitrogen atom, Y is oxygen atom, U is carbon atom, A is Formula (A-1), and B is Formula (B-2) can be prepared by the following process:

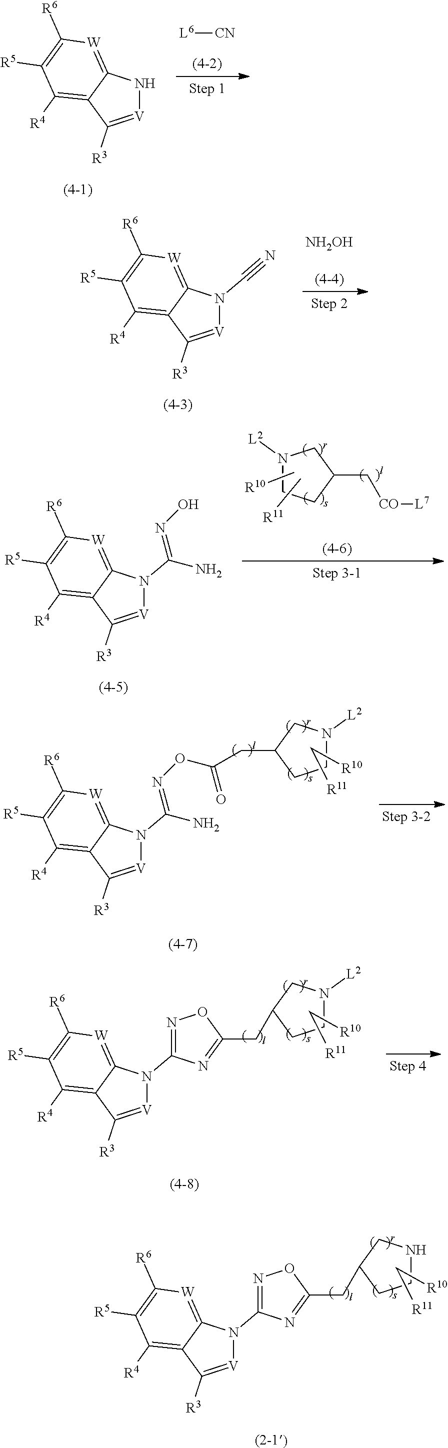

wherein l, r, s, V, W, R³, R⁴, R⁵, R⁶, R¹⁰, R¹¹ and L² are as defined above, L⁶ is a leaving group, and L⁷ is hydroxy group or a leaving group.

Step 1 is a cyanation step. The leaving group of L⁶ used herein includes, for example, bromine and p-toluenesulfonyl group. The base used herein is one or a mixture of two or more bases selected from the group consisting of, for example, trimethylamine, triethylamine, DMAP (i.e. 4-N,N-dimethylaminopyridine), pyridine, potassium tert-butoxide, butyllithium, sodium hydride, lithium hexamethyldisilazide, and cesium carbonate. The reaction temperature is usually about −80° C. to about 100° C., and preferably about 0° C. to about 80° C. The solvent used herein includes, for example, aromatic hydrocarbons such as benzene, toluene, and xylene; ethers such as diethyl ether, tetrahydrofuran, cyclopentyl methyl ether and dioxane; halogenated hydrocarbons such as methylene chloride and chloroform; alcohols such as ethanol, isopropanol, and ethylene glycol; ketones such as acetone and methyl ethyl ketone; ethyl acetate; acetonitrile; N,N-dimethylformamide; and dimethylsulfoxide. These solvents may be used alone or in a mixture of two or more.

Step 2 is a reaction to obtain an amidinoxime compound by reacting cyano group with hydroxylamine. The reaction can be carried out in the presence of a base as appropriate, and the base specifically includes alkali hydroxides such as sodium hydroxide and potassium hydroxide; alkaline carbonates such as sodium carbonate and potassium carbonate; alkaline bicarbonates such as sodium bicarbonate and potassium bicarbonate; and organic bases such as triethylamine, tributylamine, diisopropylethylamine, and N-methylmorpholine. The solvent used herein includes, for example, aromatic hydrocarbons such as benzene, toluene, and xylene; ethers such as diethyl ether, tetrahydrofuran, cyclopentyl methyl ether, and dioxane; halogenated hydrocarbons such as methylene chloride and chloroform; alcohols such as ethanol, isopropanol, and ethylene glycol; ketones such as acetone and methyl ethyl ketone; ethyl acetate; acetonitrile; N,N-dimethylformamide; dimethylsulfoxide; and water. These solvents may be used alone or in a mixture of two or more. The reaction temperature is usually about 0° C. to about 150° C., and preferably 20° C. to about 80° C.

Step 3 is a condensation step (Step 3-1) followed by a cyclization step (Step 3-2). In specific, the compound of Formula (4-5) can be reacted with the reactive derivative of Formula (4-6) in the presence of a suitable additive agent such as a base to give the compound of Formula (4-7), and then the compound of Formula (4-7) can be cyclized to give the compound of Formula (4-8).

### Condensation Step (Step 3-1)

The reactive derivative of (4-6) includes a carboxylic acid compound, and an alkyl ester thereof (in particular, methyl ester), an active ester thereof, an acid anhydride thereof and an acid halide thereof (including an acid derivative wherein the halide is replaced with another leaving group which is a halide equivalent). In case that the derivative (4-6) is a carboxylic acid compound (i.e. L⁷ is hydroxy group), the reaction can be carried out in the presence of a condensing agent such as 1,3-dicyclohexylcarbodiimide, 1-ethyl-3-(3-dimethylamino-propyl)carbodiimide hydrochloride, N,N′-carbonyldiimidazole, benzotriazol-1-yloxytris(dimethylamino)phosphonium hexa-fluorophosphate, N,N′-carbonyldisuccinimide, 1-ethoxycarbonyl-2-ethoxy-1,2-dihydroquinoline, diphenylphosphoryl azide, and propanephosphonic anhydride. In addition, in case that 1,3-dicyclohexylcarbodiimide or 1-ethyl-3-(3-dimethylaminopropyl)carbodiimide hydrochloride is used as the condensing agent, N-hydroxysuccinimide, 1-hydroxybenzotriazole, 3-hydroxy-1,2,3-benzotriazin-4(3H)-one, N-hydroxy-5-norbornene-2,3-dicarboxylmide, etc. may be added to the reaction.

In case that the derivative (4-6) is an active ester, the active ester specifically includes p-nitrophenyl ester, pentachlorophenyl ester, pentafluorophenyl ester, N-hydroxysuccinimide ester, N-hydroxyphthalimide ester, 1-hydroxybenzotriazole ester, 8-hydroxyquinoline ester, 2-hydroxyphenyl ester, etc.

In case that the derivative (4-6) is an acid anhydride, the acid anhydride specifically includes a symmetrical acid anhydride and a mixed acid anhydride. The mixed acid anhydride specifically includes a mixed acid anhydride with an alkyl chlorocarbonate such as ethyl chlorocarbonate and isobutyl chlorocarbonate, a mixed acid anhydride with an aralkyl chlorocarbonate such as benzyl chlorocarbonate, a mixed acid anhydride with an aryl chlorocarbonate such as phenyl chlorocarbonate, and a mixed acid anhydride with an alkanoic acid such as isovaleric acid and pivalic acid.

The present reaction can be carried out in the presence or absence of a solvent. The solvent used herein should be optionally selected depending on the types of starting compounds, etc., and for example, aromatic hydrocarbons such as benzene, toluene, and xylene; ethers such as diethyl ether, tetrahydrofuran, dioxane, and cyclopentyl methyl ether; halogenated hydrocarbons such as methylene chloride and chloroform; ketones such as acetone and methyl ethyl ketone; ethyl acetate; acetonitrile; dimethylformamide; and dimethylsulfoxide. These solvents may be used alone or in a mixture of two or more.

The reaction can be carried out in the presence of a base as appropriate, and the base includes alkali hydroxides such as sodium hydroxide and potassium hydroxide; alkaline carbonates such as sodium carbonate and potassium carbonate; alkaline bicarbonates such as sodium bicarbonate and potassium bicarbonate; and organic bases such as triethylamine, tributylamine, diisopropylethylamine, and N-methylmorpholine. In order to also use the compound of Formula (4-5) as a base, an excess amount of the compound may be used.

The reaction temperature depends on the types of the starting compound used herein or other factors; and it is typically about −30° C. to about 200° C., and preferably about −10° C. to about 150° C.

In case that the derivative (4-6) is an acid halide (including an acid derivative wherein the halide is replaced with another leaving group which is a halide equivalent), L⁷ includes, for example, halogen atoms (such as chlorine, bromine, and iodine) and detachable groups like halogen atoms (e.g. alkylsulfonyloxy groups such as methanesulfonyloxy group, and arylsulfonyloxy groups such as benzenesulfonyloxy group and p-toluenesulfonyloxy group). L⁷ is preferably halogen atoms (in particular, chlorine and bromine), methanesulfonyloxy group or trifluoromethane-sulfonyloxy group.

The present reaction is carried out in the presence or absence of a solvent. The solvent used herein should be optionally selected depending on the types of starting compounds, etc., and for example, aromatic hydrocarbons such as benzene, toluene, and xylene; ethers such as diethyl ether, tetrahydrofuran, dioxane, and cyclopentyl methyl ether; halogenated hydrocarbons such as methylene chloride and chloroform; ketones such as acetone and methyl ethyl ketone; ethyl acetate; acetonitrile; N,N-dimethylformamide; and dimethylsulfoxide. These solvents may be used alone or in a mixture of two or more.

The reaction can be carried out in the presence of a base as appropriate, and the base includes alkali hydroxides such as sodium hydroxide and potassium hydroxide; alkaline carbonates such as sodium carbonate and potassium carbonate; alkaline bicarbonates such as sodium bicarbonate and potassium bicarbonate; and organic bases such as triethylamine, tributylamine, diisopropylethylamine, and N-methylmorpholine. In order to also use the compound of Formula (4-5) as a base, an excess amount of the compound may be used.

The reaction temperature depends on the types of the starting compound used herein or other factors; and it is typically about 0° C. to about 200° C., and preferably about 20° C. to about 150° C.

### Cyclization Step (Step 3-2)

According to the disclosure of, for example, Current Organic Chemistry, (2008), 12(10), 850, the compound of Formula (4-7) can be reacted in the presence or absence of a suitable additive agent such as a base to give the compound of Formula (4-8).

The present reaction can be carried out in the presence or absence of a solvent. The solvent used herein should be optionally selected depending on the types of starting compounds, etc., and includes, for example, aromatic hydrocarbons such as benzene, toluene, and xylene; ethers such as diethyl ether, tetrahydrofuran, dioxane, and cyclopentyl methyl ether; halogenated hydrocarbons such as methylene chloride and chloroform; ketones such as acetone and methyl ethyl ketone; ethyl acetate; acetonitrile; N,N-dimethylformamide; dimethylsulfoxide; and acetic acid. These solvents may be used alone or in a mixture of two or more.

The base used herein includes, for example, alkaline carbonates such as sodium carbonate and potassium carbonate; alkaline bicarbonates such as sodium bicarbonate and potassium bicarbonate; alkali acetates such as sodium acetate and potassium acetate; and organic bases such as triethylamine, tributylamine, diisopropylethylamine, methylmorpholine, tetrabutylammonium fluoride, and quaternary ammonium hydroxide salts (e.g. tetramethylammonium hydroxide). The reaction temperature depends on the types of the starting compound used herein or other factors; and it is typically about 0° C. to about 200° C., preferably about 20° C. to about 110° C.

Step 4 is a deprotection reaction. The compound of Formula (4-8) can be deprotected in the same manner as in the above-described L² to give the compound of Formula (2-1′).

### (Process 5)

The compound of Formula (4-1) described in Process 4 is commercially available, or may be prepared according to known methods. The compound of Formula (4-1) wherein, for example, V is nitrogen atom and W is carbon atom [i.e. the compound of (4-1′)] can be prepared by the following process:

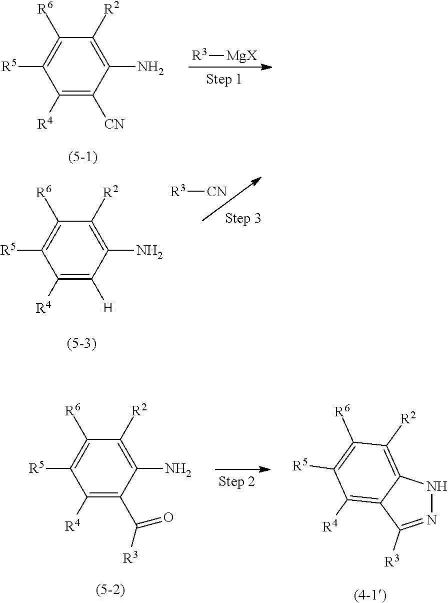

wherein R², R³, R⁴, R⁵, and R⁶ are as defined above, X is a halogen atom (for example, when R³ is methyl group, R³—MgX means methyl Grignard reagent).

Step 1 is an addition reaction of Grignard reagent to nitrile group. In specific, the compound of Formula (5-1) can be reacted with R³—MgX, and the resultant imine can be hydrolyzed by an acid to give the compound of Formula (5-2).

The solvent used herein should be optionally selected depending on the types of starting compounds, etc., and for example, hydrocarbons such as hexane and n-heptane; aromatic hydrocarbons such as benzene, toluene, and xylene; and ethers such as diethyl ether, tetrahydrofuran, dioxane, and cyclopentyl methyl ether. These solvents may be used alone or in a mixture of two or more.

The acid used herein includes mineral acids such as hydrochloric acid, hydrobromic acid, hydroiodic acid, and sulfuric acid; and preferably hydrochloric acid. The reaction temperature is usually about −80° C. to about 120° C., and preferably about −40° C. to about 60° C.

In Step 2, the amino group of the compound of Formula (5-2) can be diazotized in the presence of an acid, and the resultant diazonium salt can be reduced to make an indazole ring to give the compound of Formula (4-1′).

The acid used herein includes, for example, hydrochloric acid, hydrobromic acid, hydroiodic acid, sulfuric acid, and tetrafluoroboric acid; and preferably hydrochloric acid, sulfuric acid, and tetrafluoroboric acid.

The diazotization agent used herein includes, for example, nitrite salts such as sodium nitrite and potassium nitrite, and nitrite esters such as pentyl nitrite and isoamyl nitrite; and preferably sodium nitrite.

The reducing agent used herein includes, for example, tin (II) chloride, sodium sulfite, sodium nitrite, sodium dithionite, and sodium thiosulfate.

The reaction temperature is usually about −40° C. to about 80° C., and preferably about −20° C. to about 20° C.

The solvent used herein includes the above-mentioned acids, and additionally includes, for example, aromatic hydrocarbons such as benzene, toluene, and xylene; ethers such as diethyl ether, tetrahydrofuran, cyclopentyl methyl ether, and dioxane; halogenated hydrocarbons such as methylene chloride and chloroform; alcohols such as methanol, ethanol, isopropanol, and ethylene glycol; ethyl acetate; acetonitrile; and water. These solvents may be used alone or in a mixture of two or more.

Step 3 is Sugasawa reaction. The compound of Formula (5-3) can be reacted with a nitrile derivative (defined as R³—CN) in the presence of Lewis acid to give the compound of Formula (5-2).

The Lewis acid used herein includes, for example, zinc chloride, tin (IV) chloride, titanic chloride, aluminum chloride, boron trichloride, and gallium trichloride. These Lewis acids may be used alone or in a mixture of two or more. The Lewis acid used herein is preferably a combination of boron trichloride and aluminum chloride, or a combination of boron trichloride and gallium trichloride.

The reaction temperature is usually about −20° C. to about 200° C., preferably about −10° C. to about 150° C.

The solvent used herein includes, for example, aromatic hydrocarbons such as benzene, toluene, and xylene; ethers such as diethyl ether, tetrahydrofuran, cyclopentyl methyl ether and dioxane; halogenated hydrocarbons such as methylene chloride, chloroform, and 1,2-dichloroethane; ethyl acetate; acetonitrile; and N,N-dimethylformamide. These solvents may be used alone or in a mixture of two or more.

### (Process 6)

The compound of Formula (1) can also be prepared by the following process in case that, for example, B is Formula (B-2); and D is an optionally-substituted C₁₋₆ alkyl group, an optionally-substituted C₃₋₆ alkenyl group, an optionally-substituted C₃₋₆ alkynyl group, an optionally-substituted C₃₋₈ monocyclic, C₇₋₁₀ bicyclic or C₇₋₁₂ tricyclic cycloalkyl group, or an optionally-substituted. C₅₋₈ monocyclic or C₇₋₁₀ bicyclic cycloalkenyl group [i.e. Compound (1″)]:

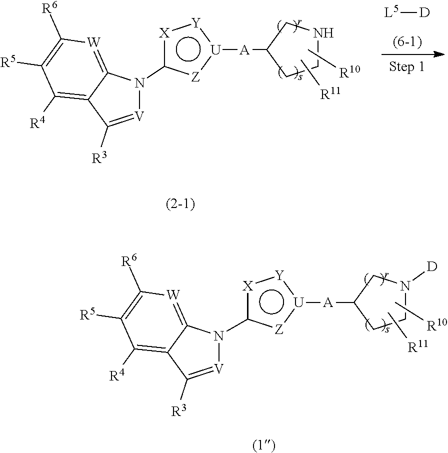

wherein r, s, A, U, V, W, X, Y, Z, R³, R⁴, R⁵, R⁶, R¹⁰ and R¹¹ are as defined above, and L⁵ is oxo group (provided that when L⁵ is attached to the primary carbon atom of D, L⁵ forms a formyl group with the attached carbon atom) or a leaving group.

In case that L⁵ is a leaving group, Step 1 is an alkylation reaction. The compound of Formula (2-1) and the compound of Formula (6-1) can be reacted in the same manner as in 1) Alkylation step of Processes 2 and 3 to give the compound of Formula (1″).

In case that L⁵ is oxo group, Step 1 is a reductive alkylation reaction. The compound of Formula (2-1) and the compound of Formula (6-1) can be reacted in the same manner as in 2) Reductive alkylation step of Processes 2 and 3 to give the compound of Formula (1″).

### (Process 7)

The compound of Formula (1) can also be prepared by the following process in case that, for example, B is Formula (B-2) and D is Formula (R¹²-3) [i.e. Compound (1′″)]:

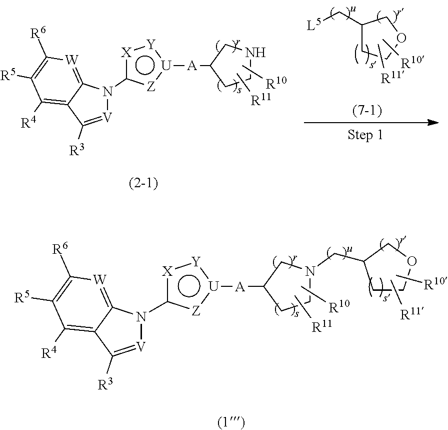

wherein r, s, r′, s′, u, A, U, V, W, X, Y, Z, R², R⁴, R⁵, R⁶, R¹⁰, R¹¹, R¹⁰′, and ¹¹′ are as defined above, and L⁵ is oxo group [provided that when L⁵ is attached to the primary carbon atom in Formula (7-1), L⁵ forms a formyl group with the attached carbon atom] or a leaving group.

In case that L⁵ is a leaving group, Step 1 is an alkylation reaction. The compound of Formula (2-1) and the compound of Formula (7-1) can be reacted in the same manner as in 1) Alkylation step of Processes 2 and 3 to give the compound of Formula (1′″).

In case that L⁵ is oxo group, Step 1 is a reductive alkylation reaction. The compound of Formula (2-1) and the compound of Formula (7-1) can be reacted in the same manner as in 2) Reductive alkylation step of Processes 2 and 3 to give the compound of Formula (1′″).

### (Process 8)

The compound of Formula (1) can also be prepared by the following process in case that, for example, X is nitrogen atom, Z is nitrogen atom, Y is oxygen atom, and U is carbon atom [i.e. Compound (1′″)]:

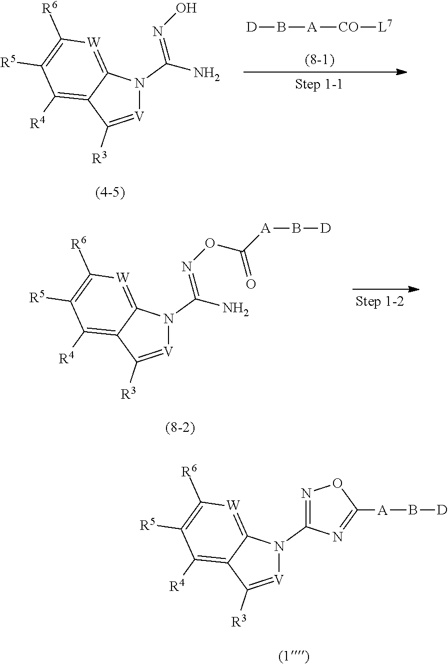

wherein A, B, D, V, W, R³, R⁴, R⁵, R⁶ and L⁷ are as defined above.

Step 1-1 is a condensation reaction, and Step 1-2 is a subsequent cyclization reaction. In the same manner as in Steps 3-1 and 3-2 of Process 4, the compound of Formula (4-5) and the compound of Formula (8-1) can be condensed and then cyclized to give the compound of Formula (1′″).

### (Process 9)

The compound of Formula (1) can also be prepared by the following process in case that, for example, X is nitrogen atom, Z is nitrogen atom, Y is oxygen atom, U is carbon atom, A is Formula (A-3), and B is Formula (B-1) [i.e. the compound of Formula (1′″″)]:

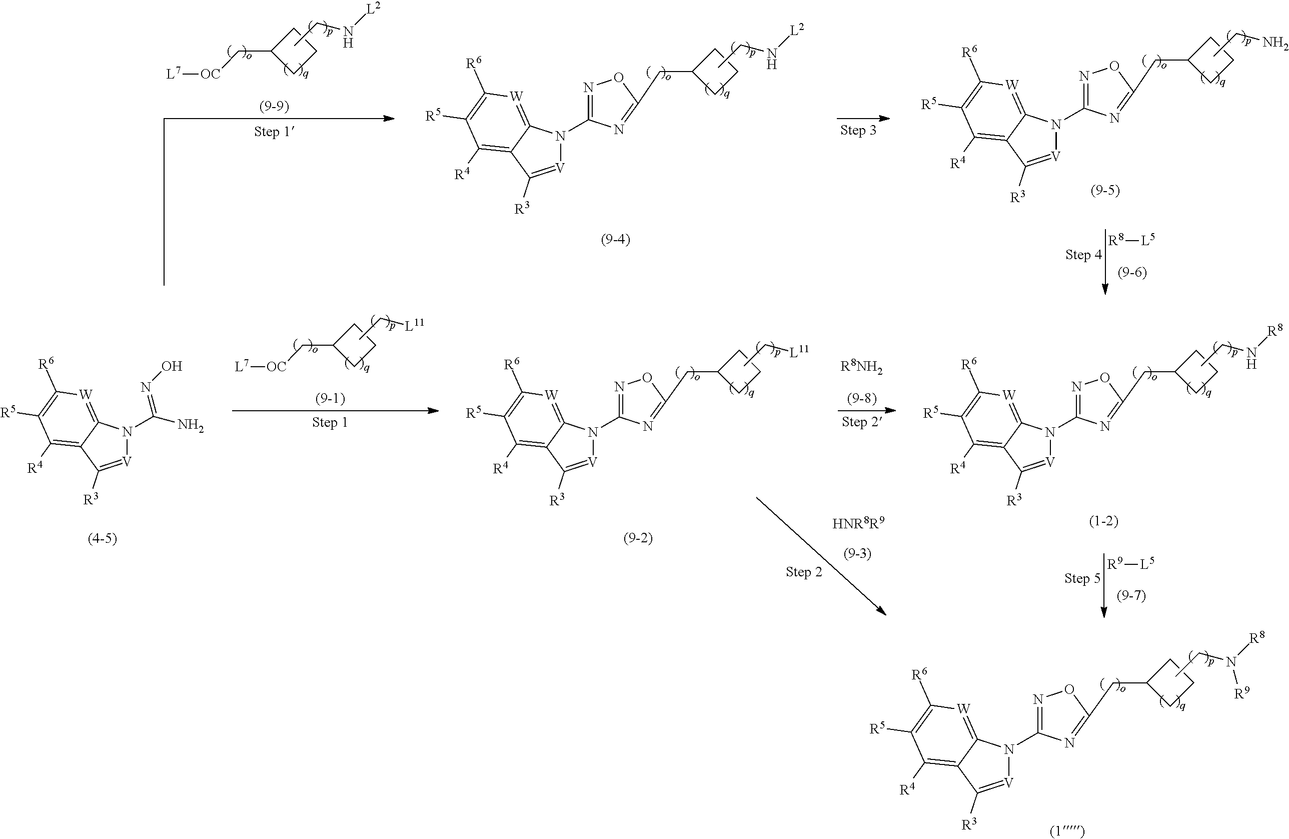

wherein o, p, q, V, W, R³, R⁴, R⁵, R⁶, R⁸, R⁹, L², and L⁷ are as defined above; and L⁵ and L¹¹ are independently oxo group (provided that when L⁵ or L¹¹ is attached to the primary carbon atom, L⁵ or L¹¹ forms a formyl group with the attached carbon atom) or a leaving group.

Step 1 and Step 1′ are a condensation reaction followed by a cyclization reaction. In the same manner as in Steps 3-1 and 3-2 of Process 4, the compound of Formula (4-5) can be reacted with the compound of Formula (9-1) or Formula (9-9) to give the compound of Formula (9-2) or (9-4), respectively.

In case that L¹¹ is a leaving group, Step 2 and Step 2′ is an alkylation reaction. In the same manner as in 1) Alkylation step of Processes 2 and 3, the compound of Formula (9-2) can be reacted with the compound of Formula (9-3) or Formula (9-8) to give the compound of Formula (1′″″) or Formula (1-2), respectively.

In case that L¹¹ is oxo group, Step 2 and Step 2′ are a reductive alkylation reaction. In the same manner as in 2) Reductive alkylation step of Processes 2 and 3, the compound of Formula (9-2) can be reacted with the compound of Formula (9-3) or Formula (9-8) to give the compound of Formula (1′″″) or Formula (1-2), respectively.

Step 3 is a deprotection reaction. The compound of Formula (9-4) is deprotected in the same manner as in the above-mentioned L² to give the compound of Formula (9-5).

In case that L⁵ is a leaving group, Step 4 and Step 5 are an alkylation reaction. In the same manner as in 1) Alkylation step of Processes 2 and 3, the compounds of Formula (9-5) and Formula (9-6), or the compounds of Formula (1-2) and Formula (9-7) can be reacted to give the compound of Formula (1-2) or Formula (1′″″), respectively.

In case that L⁵ is oxo group, Step 4 and Step 5 is a reductive alkylation reaction. In the same manner as in 2) Reductive alkylation step of Processes 2 and 3, the compounds of Formula (9-5) and (9-6), or the compounds of Formula (1-2) and Formula (9-7) can be reacted to give the compound of Formula (1-2) or Formula (1′″″), respectively.

### (Process 10)

The compound of Formula (2-1):

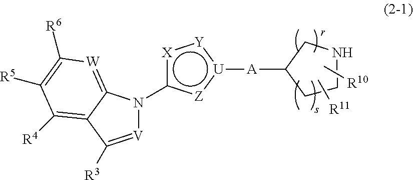

wherein r, s, A, V, W, R², R⁴, R⁵, R⁶, R¹⁰, and R¹¹ are as defined above
can be prepared, for example, in the same manner as in Reference Example 062 in case that X and Y are nitrogen atom, Z is oxygen atom, and U is carbon atom; in the same manner as in Reference Example 064 in case that X is oxygen atom, Y and Z are nitrogen atom, and U is carbon atom; and
in the same manner as in Reference Example 063 in case that X is oxygen atom, Y is nitrogen atom, and Z and U are carbon atom.

In the above-explained processes, when any functional groups other than the reactive site may be reacted under the explained conditions or be unsuitable to carry out the explained processes, the desired compound can be obtained by protecting the groups except the reactive site, carrying out the reaction, and then deprotecting it. The protecting group used herein includes, for example, typical protecting groups described in the above-mentioned Protective Groups in Organic Synthesis and the like. In specific, the protecting group of amine includes, for example, ethoxycarbonyl, tert-butoxycarbonyl, acetyl, and benzyl; and that of hydroxy group includes, for example, tri(loweralkyl)silyl, acetyl, and benzyl.

The protecting groups can be introduced and deprotected according to commonly used methods in synthetic organic chemistry (for example, see, the above-mentioned Protective Groups in Organic Synthesis) or other similar methods.

In addition, when the functional groups of the intermediates and the desired compounds in each process mentioned above are modified appropriately, different compounds in the present invention can be prepared. The functional group can be modified according to conventional general-methods (for example, see, Comprehensive Organic Transformations, R. C. Larock, 1989).

The starting materials and the intermediates in each of the above processes are well-known compounds or can be synthesized from well-known compounds according to well-known methods.

The intermediates and the desired compounds in each of the above processes can be isolated and purified according to commonly-used purification methods in synthetic organic chemistry such as neutralization, filtration, extraction, washing, drying, concentration, recrystallization, and various types of chromatography. In addition, the intermediates may be used in the next reaction without purification.

The optical isomers such as enantiomers, planar-chiral forms, and axially chiral forms used herein can be resolved/isolated by using well-known resolving steps (e.g. methods using an optically active column, and fractionated crystallization) in a suitable step in the above processes. In addition, optically active substances may also be used as a starting material herein.

In order to optically resolve the present compound or an intermediate thereof having a basic group, the compound may, for example, form a salt with an optically active acid (e.g. monocarboxylic acids such as mandelic acid, N-benzyloxyalanine, and lactic acid; dicarboxylic acids such as tartaric acid, o-diisopropylidene tartrate, and malic acid; and sulfonic acids such as camphorsulfonic acid and bromocamphorsulfonic acid) in an inert solvent (e.g. alcohol solvents such as methanol, ethanol, and 2-propanol; ether solvents such as diethyl ether; ester solvents such as ethyl acetate; aromatic hydrocarbon solvents such as toluene; acetonitrile; and a mixed solvent thereof).

In case that the present compound or an intermediate thereof has an acidic substituent such as carboxyl group, the compound may be optically resolved by forming a salt thereof with an optically active amine (e.g. organic amines such as α-phenethylamine, kinin, quinidine, cinchonidine, cinchonine, and strychnine).

The temperature for forming the salt may be in the range of room temperature to boiling point of the solvent. In order to improve the optical purity, it is desirable to once raise the temperature to around the boiling point of the solvent. The solvent containing the crystallized salt can be optionally cooled before the filtration to raise the yield thereof. The amount of the optically active acid or amine used herein is in the range of about 0.5 equivalent to about 2.0 equivalents, and preferably around 1 equivalent per the substrate. The crystal can be optionally recrystallized in an inert solvent (e.g. alcohol solvents such as methanol, ethanol, and 2-propanol; ether solvents such as diethyl ether; ester solvents such as ethyl acetate; aromatic hydrocarbon solvents such as toluene; acetonitrile; and a mixed solvent thereof) to obtain an optically active salt with high purity. If necessary, the resultant salt can be treated with an acid or base in a conventional method to obtain a free form thereof.

The compound of Formula (1) can be obtained in the form of a free base or acid addition salt, depending on the types of the functional group in the formula, selection of the starting compound, and treatments/conditions of the reaction. Such free base or acid addition salt can be transformed into the compound of Formula (1) according to conventional methods. Meanwhile, the compound of Formula (1) can be treated with various acids by using conventional methods to obtain an acid addition salt thereof.

When it is necessary to obtain a salt of the present compound, if the present compound is given in the form of a salt, the resultant salt can be directly purified. On the other hand, if the present compound is given in a free form, the compound can be transformed to a salt thereof according to a conventional method by dissolving or suspending the free form in a suitable organic solvent, and then adding an acid or base thereto.

Furthermore, the present compound and a pharmaceutically acceptable salt thereof may exist in an addition form with water or various solvents, which are also comprised in the present invention. Moreover, the present invention may encompass all tautomers of the present compound, all possible stereoisomers of the present compound, all optical isomers of the present compound, and all aspects of crystals of the present compound.

The present compound or a pharmaceutically acceptable salt thereof has a strong affinity and agonistic activity for serotonin-4 receptor, which is explained below, and thus expected to be a useful medicament for patients suffering from diseases or symptoms which are desired and/or required to be treated with an agonistic action or partial agonistic action for serotonin-4 receptor.

The diseases or symptoms which are desired and/or required to be treated with an agonistic action or partial agonistic action for serotonin-4 receptor include, for example, the following (i) to (v):

(i) neuropsychiatric diseases such as Alzheimer-type dementia, Lewy body dementia, vascular dementia, depression, posttraumatic stress disorder (PTSD), memory impairment, anxiety, and schizophrenia;
(ii) digestive system diseases such as irritable bowel syndrome, atonic constipation, habitual constipation, chronic constipation, constipation induced by drugs (e.g. morphine and antipsychotic drugs), constipation associated with Parkinson's disease, constipation associated with multiple sclerosis, constipation associated with diabetes mellitus, and constipation or dyschezia caused by contrast materials taken as a pretreatment for endoscopic examinations or barium enema X-ray examinations;
(iii) digestive system diseases such as functional dyspepsia, acute/chronic gastritis, reflux esophagitis, gastric ulcer, duodenal ulcer, gastric neurosis, postoperative paralytic ileus, senile ileus, non-erosive reflux disease, NSAID ulcer, diabetic gastroparesis, postgastrectomy syndrome, and intestinal pseudo-obstruction;
(iv) digestive system symptoms such as the digestive system diseases mentioned in the above (ii) and (iii), scleroderma, diabetes mellitus, anorexia in esophageal/biliary-tract diseases, nausea, emesis, bloating, epigastric discomfort, abdominal pain, heartburn, and belching; and
(v) urinary system diseases associated with dysuria such as urinary tract obstruction and prostatic hyperplasia.

The present compound or a pharmaceutically acceptable salt thereof is useful as a medicament for treating or preventing especially the neuropsychiatric diseases such as Alzheimer-type dementia mentioned in the above (i) because the compound shows an excellent 5-HT₄ receptor agonist activity and brain penetration.

The present compound or a pharmaceutically acceptable salt thereof may be orally or parenterally administered (e.g. intravenous or subcutaneous administration; infusions; intramuscular injections; subcutaneous injections; intranasal formulations; eye-drops, suppositories; and transdermal formulations such as ointments, creams, and lotions) for medical use. A formulation for oral administration includes, for example, tablets, capsules, pills, granules, powders, liquids, syrups and suspensions; and a formulation for parenteral administration includes, for example, injectable aqueous or oleaginous suspensions, ointments, creams, lotions, aerosols, suppositories, and adhesive skin patches.

These formulations can be formulated by using conventionally-known techniques, and may comprise conventionally-acceptable carriers, excipients, binders, stabilizers, lubricants, disintegrants, etc. Moreover, the formulation for injection may further comprise an acceptable buffer, solubilizing agent, isotonic agent, etc. The formulation may also optionally comprise flavoring agent.

The excipient used herein includes, for example, organic excipients such as sugar derivative (e.g. lactose, white soft sugar, glucose, mannitol, and sorbitol); starch derivatives (e.g. corn starch, potato starch, α-starch, dextrin, and carboxymethyl starch); cellulose derivatives (e.g. crystalline cellulose, low-substituted hydroxy-propylcellulose, hydroxypropyl methylcellulose, carboxymethylcellulose, carboxymethyl cellulose calcium, and internally-cross-linked carboxymethylcellulose sodium); acacia; dextran; and pullulan; and inorganic excipients such as silicate derivatives (e.g. light anhydrous silicic acid, synthetic aluminum silicate, and magnesium aluminometasilicate); phosphates (e.g. calcium phosphate); carbonates (e.g. calcium carbonate); and sulfates (e.g. calcium sulfate).

The lubricant used herein includes, for example, stearic acid; metallic stearate such as calcium stearate, and magnesium stearate; talc; colloid silica; waxes such as VEEGUM and spermaceti; boric acid; adipic acid; sulfates such as sodium sulfate; glycol; fumaric acid; sodium benzoate; DL-leucine; fatty acid sodium salt; lauryl sulfates such as sodium lauryl sulfate and magnesium lauryl sulfate; silicates such as anhydrous silicic acid and silicic acid hydrate; and the above-mentioned starch derivatives.

The binder used herein includes, for example, polyvinylpyrrolidone, macrogol, and the substances defined in the above-mentioned excipient.

The disintegrant used herein includes, for example, the substances defined in the above-mentioned excipient, and chemically-modified starches/celluloses such as croscarmellose sodium, sodium carboxymethyl starch, and cross-linked polyvinylpyrrolidone.

The stabilizer used herein includes, for example, p-hydroxybenzoates such as methylparaben and propylparaben; alcohols such as chlorobutanol, benzyl alcohol, and phenylethyl alcohol; benzalkonium chloride; phenols such as phenol and cresol; thimerosal; dehydroacetic acid; and sorbic acid.

The flavoring agent used herein includes, for example, commonly-used sweeteners, acidulants, and flavors.

A tablet for oral administration may comprise an excipient together with various disintegrants as well as granulating binders. Furthermore, a lubricant is often very useful for tablet formulation. A similar type of the solid composition may be used as a bulking agent of a gelatin capsule which may be combined by various ingredients, preferably lactose (milk sugar) or high-molecular-weight polyethylene glycol.

The active ingredient of aqueous suspension and/or elixir for oral administration may be combined with a diluent together with various sweetening agents, flavoring agents, coloring agents or dyes, or if desired, emulsifiers and/or suspending agents. The diluent includes water, ethanol, propylene glycol, glycerin and a mixture thereof. The diluent is conveniently included in feed or drinking water for animal in a concentration of 5 ppm to 5000 ppm, and preferably 25 ppm to 5000 ppm.

A solution of the active ingredient for sterile injection may be typically prepared for parenteral administration (e.g. intramuscular, intraperitoneal, subcutaneous and intravenous use). A solution of the present compound in, for example, sesame oil, peanut oil or aqueous propylene glycol may be used. If necessary, the aqueous solution may be appropriately adjusted or buffered to a suitable pH, or prepared into an isotonic solution with a liquid diluent. The aqueous solution can also be used for intravenous injection. The oil solution can be also used for intra-articular, intramuscular and subcutaneous injections. All of these solutions may be prepared under sterile conditions by using conventional formulation techniques known to those skilled in the art.

The present compound or a pharmaceutically acceptable salt thereof for the intranasal or inhalation administration may be provided in the solution or suspension form squeezed out or released by a patient from a pump spray vessel, or as an aerosol spray from a pressurized vessel or a nebulizer with using an appropriate propellant including, for example, dichlorodifluoromethane, trichlorofluoromethane, dichlorotetrafluoroethane, carbon dioxide and other appropriate gases. A dosage unit in the pressurized aerosol can be determined by a bulb which provides a certain measured amount of the active ingredient. A solution or suspension of the active compound may be contained in the pressurized vessel or nebulizer.

A capsule and cartridge for an inhaler or insufflator (e.g., prepared from gelatin) may be formulated to contain the present compound and a powder composition of appropriate powder bases including, for example, lactose and starch.

The present compound or a pharmaceutically acceptable salt thereof may be also formulated in a composition for the anus such as a suppository or retention enema comprising conventional suppository bases including, for example, cacao butter and other glycerides.

A dosage of the present compound or a pharmaceutically acceptable salt thereof depends on conditions, ages, administration methods, etc., and for example, the dosage is 0.01 mg (preferably 1 mg) as a lower limit and 5000 mg (preferably 500 mg) as an upper limit per day at one time or in several divided doses for adults for oral administration, preferably depending on conditions. It is expected to be effective in 0.01 mg (preferably 0.1 mg) as a lower limit and 1000 mg (preferably 30 mg) as an upper limit per day at one time or in several divided doses for adults for intravenous administration depending on conditions.

The present compound or a pharmaceutically acceptable salt thereof, or a pharmaceutical composition or formulation containing the present compound may be optionally administered in combination with other medicaments in order to treat the diseases defined herein which are required to be treated with an agonistic action or partial agonistic action for serotonin-4 receptor.

In specific, the present compound or a pharmaĉeutically acceptable salt thereof, or a pharmaceutical composition or formulation containing the present compound is expected to show further efficacy in treating the various neuropsychiatric diseases mentioned in the above (i), especially Alzheimer-type dementia, by combining at least one of the following medicaments:

acetylcholinesterase inhibitors such as donepezil, galantamine, rivastigmine, SNX-001 and NP-61; cholinesterase inhibitors such as huperzine A; NMDA receptor antagonist such as memantine, dimebon and neramexane; 5-HT6 receptor antagonists such as PF-5212365 (SAM-531), SB-742457, LU-AE58054, AVN-322, PF-05212377 (SAM-760) and AVN101; α7nAChR agonists such as TC-5619, EVP-6124 and GTS-21; α4β2nACh receptor agonists such as AZD-1446 and CHANTIX (varenicline); nAChR agonists such as ABT-089; AMPA receptor agonists such as CX-717 and LY-451395; histamine H3 antagonists such as ABT-288, SAR-110894 and PF-03654746; muscarinic M1 receptor agonists such as MCD-386 and GSK-1034702; PDE4 inhibitors such as etazolate; PDE9 inhibitors such as PF-04447943; histone deacetylase inhibitors such as EVP-0334; σ1 receptor agonists such as Anavex-2-73; γ-secretase inhibitors (GSI) such as BMS-708163, NIC5-15, ELND-006, and MK-0752; γ-secretase modulators (GSM) such as E-2212 and CHF-5074; Aβ human monoclonal antibodies such as bapineuzumab, solanezumab, PF-4360365 (ponezumab), gantenerumab (R-1450), BAN-2401, MABT-5102A, RG-7412 and GSK-933776A; Aβ vaccines such as ACC-001 (PF-05236806), AD-02, CAD-106, V-950, UB-311 and ACI-24; human immunoglobulins such as GAMMAGARD; Aβ aggregation inhibitors such as ELND-005 (AZD-103), PBT-2, NRM-8499 and Exebryl-1; tau aggregation inhibitors such as TRx-0014 and LMTX; BACE inhibitors such as ACI-91, posiphen, CTS-21166, HPP-854 and LY-2886721; tyrosine kinase inhibitors such as masitinib; GSK-3β inhibitors/tau kinase inhibitors such as NP-12; RAGE fusion proteins such as TTP-4000; ApoA-I/HDL-C elevations such as RVX-208; other various agents showing neuroprotective action such as SK-PC-B70M, T-817MA, davunetide, HF-0220, PF-4494700, PYM-50028, CERE-110, ASP-0777, TAK-065, and AAD-2004; and other medicaments used for treating various types of dementia.

### EXAMPLE

Hereinafter, the present inventions are illustrated in more detail with Reference Examples and Examples, but the technical scope of the present inventions should not be construed to be limited thereto. The compounds were identified by proton NMR spectrum (¹H-NMR), LC-MS, etc. Tetramethylsilane was used as an internal standard for the NMR spectrum.

In addition, the compound names shown in the following Reference Examples and Examples do not necessarily correspond to those of IUPAC nomenclature.

The following abbreviations may be optionally used in Reference Examples and Examples.

### THF: Tetrahydrofuran

NaBH(OAc)₃: Triacetoxysodium borohydride

### (Boc)₂O: Di-tert-butyldicarbonate

Pd(OH)₂: Palladium hydroxide

### DMF: N,N-dimethylformamide

WSCI.HC1: 1-(3-Dimethylaminopropyl)-3-ethylcarbodiimide hydrochloride
HOBt.H₂O: 1-hydroxybenzotriazole monohydrate
NMP: 1-Methyl-2-pyrrolidone
TFA: Trifluoroacetic acid
FA: Formic acid
CDCl₃: Deuterated chloroform
CD₃OD: Deuterated methanol
DMSO-d₆: Deuterated dimethylsulfoxide

### Me: Methyl

### Et: Ethyl

ⁿPr: Normal propyl
ⁱPr: Isopropyl
^{c}Pr: Cyclopropyl
ⁿBu: Normal butyl
ⁱBu: Isobutyl
^{c}Bu: Cyclobutyl

### Ph: Phenyl

### Ac: Acetyl

### Ms: Mesyl

### Ts: Tosyl

Boc: tert-butoxycarbonyl

### Pd—C: Palladium-carbon

NaBH₃(CN): Sodium cyanoborohydride

### Cbz or Z: Benzyloxycarbonyl

CH₂Cl₂:Methylene chloride
Ns: Nosyl(2-nitrobenzenesulfonyl)

### SEM: 2-(Trimethylsilyl)ethoxymethyl

### NEt₃: Triethylamine

CDI: N,N′-carbonylimidazole
TBAF: Tetrabutylammonium fluoride

### MeO or OMe: Methoxy

BBr₃: Boron tribromide
LiHMDS: Lithium hexamethyldisilazide
BINAP: 2,2′-Bis(diphenylphosphino)-1,1′-binaphthyl
DMAP: N,N-dimethyl-4-aminopyridine
p.o.: Peroral administration

### s: Singlet

### d: Doublet

### t: Triplet

### q: Quartet

### m: Multiplet

### br: Broad

dd: Double doublet
td: Triple doublet
J: Coupling constant

### Hz: Hertz

N: Normal (e.g. 2 N HCl means 2 normal of HCl)
M: Molar concentration (mol/L) (e.g. 2 M methylamine means
2 mol/L of methylamine solution)

### min: Minute

### atm: Atmosphere

The isolation/purification by a reverse-phase HPLC herein was carried out under the following conditions:

Condition FA: (TFA or FA as an additive)

### Instrument: Shimadzu & Gilson215

### Column: Grace Vydac C18, 200×25 mm, 5 μm

Flow rate: 30 ml/min
Column temperature: 40° C.
Moving bed A1: Distilled water (containing 0.075% TFA, v/v)
Moving bed A2: Distilled water (containing 0.2% FA, v/v)

### Moving bed B: Acetonitrile

Condition FB: (Basic or neutral condition)

### Instrument: Shimadzu & Gilson215

### Column: Durashell C18, 200×25 mm, 5 μm

Flow rate: 30 ml/min
Column temperature: 30° C.
Moving bed A1: Distilled water (containing 0.05% ammonia, v/v)
Moving bed A2: Distilled water

### Moving bed B: Acetonitrile

### Condition FC: (TFA)

### Instrument: Shimadzu & Gilson281

### Column: YMC ODS-AQ, 150×30 mm, 5 μm

Flow rate: 28 ml/min
Column temperature: 40° C.
Moving bed A: Distilled water (containing 0.075% TFA, v/v)
Moving bed B: Acetonitrile (containing 0.025% TFA, v/v)

### Condition FD: (TFA)

### Instrument: Gilson215

### Column: YMC ODS-AQ, 150×30 mm, 5 μm

Flow rate: 28 ml/min
Column temperature: 40° C.
Moving bed A: Distilled water (containing 0.075% TFA, v/v)
Moving bed B: Acetonitrile (containing 0.025% TFA, v/v)

### Condition FE: (TFA)

### Instrument: Gilson281

### Column: Synergi max RP, 150×30 mm, 5 μm

Flow rate: 25 ml/min
Column temperature: 40° C.
Moving bed A: Distilled water (containing 0.075% TFA, v/v)
Moving bed B: Acetonitrile (containing 0.025% TFA, v/v)

LC/MS analytic conditions for identifying compounds are as follows.

High performance liquid chromatograph mass spectrometer;
Measuring conditions of LCMS are as follows. The observed values of mass spectrometry, i.e. [MS (m/z)], are shown as [M+H]⁺. In case that the analyzed compound is a salt thereof, unless otherwise noted, M means a mass number of the free base thereof.

### Measurement Method A:

Detection instrument: API Agilent 1100 Series (manufactured by Applied Biosystems)
HPLC: API150EX LC/MS system (manufactured by Applied Biosystems)

### Column: YMC CombiScreen ODS-A (S-5 μm, 12 nm, 4.6×50 mm)

### Solvent: Solution A: 0.05% TFA/H₂O,

Solution B: 0.035% TFA/MeOH

### Gradient Condition:

### 0.0-1.0 min A 75% (B 25%)

1.0-4.7 min Linear gradient from A 75% to 1% (B 25% to 99%)

### 4.7-5.7 min A 1% (B 99%)

5.7-6.1 min Linear gradient from A 1% to 75% (B 99% to 25%)

### 6.1-7.1 min A 75% (B 25%)

7.1-7.2 min Linear gradient from A 75% to 100% (B 25% to 0%)
Flow rate: 2.4 mL/min

### UV: 220 nm

### Measurement Method B:

LC-MS: Waters ACQUITY™ UltraPerformance LC

### Column: Waters ACQUITY UPLC BEH Phenyl 1.7 μm, 2.1×50 mm

Solvent: Solution A: 0.05% formic acid/H₂O,
Solution B: 0.05% formic acid/CH₃CN

### Gradient Condition:

0.0 min; A/B=90:10
0.0-1.3 min; A/B=90:10-1:99 (linear gradient)
1.3-1.5 min; A/B=1:99
1.5-2.0 min; A/B=90:10
Flow rate: 0.75 mL/min

### UV: 220, 254 nm

Column temperature: 40° C.

### Measurement Method C:

LCMS: Shimadzu LCMS-2010EV

Column: Shiseido CAPCELL PAK C18 MGII (4.6 mm×50 mm)

### Solvent: Solution A: MeOH, Solution B: 0.05% TFA/H₂O

### Gradient Condition:

0.0-1.0 min; A/B=30:70
1.0-7.0 min; A/B=99:1
7.1-12.0 min; A/B=30:70
Flow rate: 2.8 mL/min

### UV: 220 nm

Column temperature: 40° C.

### Measurement Method D:

LCMS: Shimadzu LCMS-2010EV

Column: Ximate C18 3.0 m 2.1 mm×30 mm

### Solvent: Solution A: 0.019% TFA/H₂O,

Solution B: 0.038% TFA/CH₃CN

### Gradient Condition:

0.0 min; A/B=90:10
0.0-1.35 min; A/B=20:80
1.35-2.25 min; A/B=20:80
2.25-2.26 min; A/B=90:10
2.26-3.00 min; A/B=90:10
Flow rate: 0.8 mL/min

### UV: 220 nm

Column temperature: 50° C.

### Measurement Method E:

LCMS: Shimadzu LCMS-2020

Column: Phenomenex Kinetex 1.7 μm C18 (50 mm×2.10 mm)

### Solvent: Solution A: MeOH,

Solution B: 0.05% TFA/H₂O

### Gradient Condition:

0.0 min; A/B=30:70
0.0-1.90 min; A/B=99:1
1.91-3.00 min; A/B=30:70
Flow rate: 0.5 mL/min

### UV: 220 nm

Column temperature: 40° C.

The NMR measurements herein were carried out by using JEOL JNM-AL LA 300 and AL 400.

The starting compounds, reagents, and solvents used herein are commercially available, unless otherwise noted.

### Reference Example 001

### Preparation of 3-(propan-2-yl)-1H-indazole

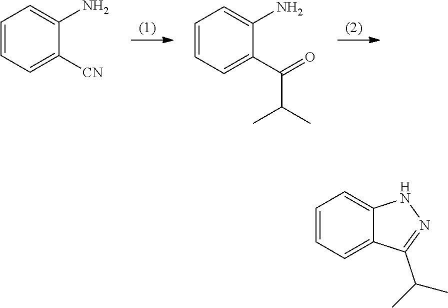

(1) 2-Aminobenzonitrile (6.5 g) was dissolved in diethyl ether (25 ml). To the solution was added dropwise 2 N isopropylmagnesium chloride in diethyl ether (76 ml) with stirring at 0° C., and then the solution was further stirred at 0° C. for 5 hours. To the reaction solution was added dropwise 10% aqueous hydrochloric acid (115 ml) with stirring at 0° C., and then the solution was further stirred for 1 hour. The reaction solution was basified by adding sodium hydroxide (19.3 g) thereto with stirring at 0° C., and the resultant solution was extracted with diethyl ether. The organic layer was washed with brine, dried over sodium sulfate and filtered, and then the filtrate was concentrated under reduced pressure to give 1-(2-aminophenyl)-2-methylpropan-1-one (8.85 g) as a brownish-red oil.

LC-MS, m/z; 164 [M+H]+

(2) The above-prepared compound (5 g) was dissolved in concentrated hydrochloric acid (25 ml). To the solution was added dropwise sodium nitrite (2.4 g) dissolved in water (12 ml) with stirring at 0° C., and then the mixed solution was further stirred at 0° C. for 1 hour. To the reaction solution was added dropwise tin (II) chloride dihydrate (16.5 g) dissolved in concentrated hydrochloric acid (12 ml), and then the solution was stirred at 0° C. for hours. The reaction solution was extracted with dichloromethane, the organic layer was washed with brine, dired over sodium sulfate and filtered, and then the filtrate was concentrated under reduced pressure. The residue was purified by silica-gel chromatography (developing solvent: hexane/ethyl acetate=2:1) to give the title compound (4.3 g) as a white solid.

LC-MS, m/z; 161 [M+H]+

The compounds in the following table (i.e. Reference Examples 002 to 012) were prepared in the same manner as in Reference Example 001 except that the 2-aminobenzonitrile and the isopropylmagnesium chloride were replaced with the corresponding starting compound and Grignard reagent defined as R³MgX wherein X is halogen atom, respectively.

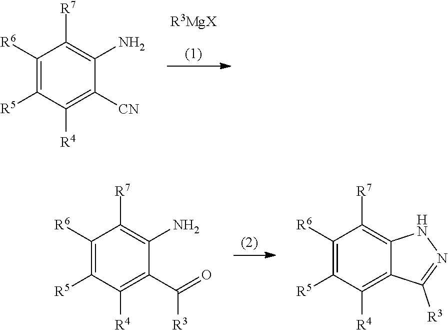

TABLE 1

| Ref. | | | | | | Compound | |
|---|---|---|---|---|---|---|---|
| Ex. | R³ | R⁴ | R⁵ | R⁶ | R⁷ | Name | LC-MS, m/z |
| 002 | Et | H | H | H | H | 3-ethyl-1H- | LC-MS, m/z; |
| | | | | | | indazole | 147 [M + H]+ |
| 003 | Et | F | H | H | H | 3-ethyl-4- | LC-MS, m/z; |
| | | | | | | fluoro-1H- | 165 [M + H]+ |
| | | | | | | indazole | |
| 004 | Et | H | F | H | H | 3-ethyl-5- | LC-MS, m/z; |
| | | | | | | fluoro-1H- | 165 [M + H]+ |
| | | | | | | indazole | |
| 005 | Et | H | H | F | H | 3-ethyl-6- | LC-MS, m/z; |
| | | | | | | fluoro-1H- | 165 [M + H]+ |
| | | | | | | indazole | |
| 006 | Et | H | H | H | F | 3-ethyl-7- | LC-MS, m/z; |
| | | | | | | fluoro-1H- | 165 [M + H]+ |
| | | | | | | indazole | |
| 007 | 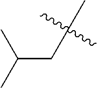 [SMILES: *CC(C)C] | H | H | H | H | 3-(2- methylpropyl)- 1H-indazole | LC-MS, m/z; 175 [M + H]+ |
| 008 | Me | H | H | H | H | 3-methyl-1H- | LC-MS, m/z; |
| | | | | | | indazole | 133 [M + H]+ |
| 009¹⁾ | 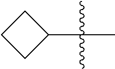 [SMILES: *C1CCC1] | H | H | H | H | 3-cyclobutyl- 1H-indazole | LC-MS, m/z; 173 [M + H]+ |
| 010 | ⁱPr | H | H | H | F | 7-fluoro-3- | LC-MS, m/z; |
| | | | | | | (propan-2- | 179 [M + H]+ |
| | | | | | | yl)-1H- | |
| | | | | | | indazole | |
| 011 | Et | H | H | MeO | H | 3-ethyl-6- | LC-MS, m/z; |
| | | | | | | methoxy-1H- | 177 [M + H]+ |
| | | | | | | indazole | |
| 012 | Et | H | H | H | Cl | 7-chloro-3- | LC-MS, m/z; |
| | | | | | | ethyl-1H- | 225 [M + HCOO]− |
| | | | | | | indazole | |

¹⁾The Grignard reagent used herein was prepared from bromocyclobutane and magnesium.

### Reference Example 013

### Preparation of 3-cyclopropyl-1H-indazole

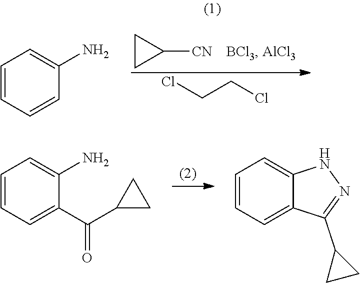

(1) To 1N boron trichloride/methylene chloride solution (100 ml) was added 1,2-dichloroethane (100 ml). The mixed solution was cooled to 0° C. to 5° C., and aniline (9.3 g) was added thereto. To the reaction solution was added cyclopropylcyanide (10 g) and aluminum chloride (14.4 g). The mixture was warmed to room temperature and methylene chloride was removed out at 70° C. The reaction solution was refluxed for 18 hours and then cooled in an ice bath, water was added thereto, and the mixture was extracted with methylene chloride (100 ml). The organic layer was dried over sodium sulfate and filtered, and the filtrate was concentrated under reduced pressure to give (2-aminophenyl)(cyclopropyl)methanone (5.0 g).

(2) The above-prepared compound was treated in the same manner as in Reference Example 001 (2) to give the title compound.

LC-MS, m/z; 159 [M+H]+

### Reference Example 014

### Preparation of 3-(methoxymethyl)-1H-indazole

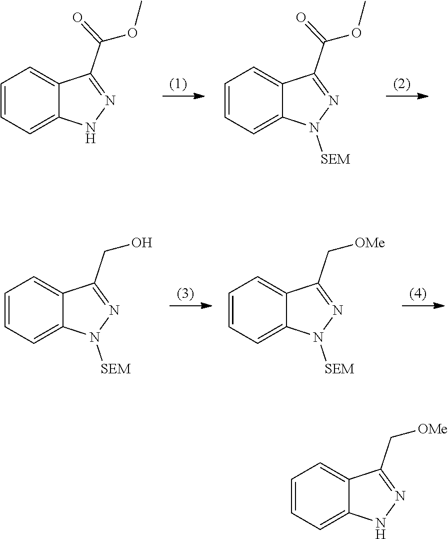 [SMILES: COC(=O)c1n[nH]c2ccccc12 ; COC(=O)c1nn(COCC[Si](C)(C)C)c2ccccc12 ; C[Si](C)(C)CCOCn1nc(CO)c2ccccc21 ; COCc1nn(COCC[Si](C)(C)C)c2ccccc12 ; COCc1n[nH]c2ccccc12]

### [SEM: 2-(Trimethylsilyl)ethoxymethyl]

(1) 1-{[2-(Trimethylsilyl)ethoxy]methyl}-1H-indazole-3-carboxylic methyl ester

To a suspension of sodium hydride (2.23 g, 55% in silicone oil) in THF (70 ml) was added dropwise indazole-3-carboxylic methyl ester (3.0 g) in THF (30 ml) at 0° C., and the mixture was stirred at the same temperature for 1 hour. To the reaction solution was added dropwise 2-(trimethylsilyl)ethoxymethyl chloride (3.62 ml) at 0° C., and the mixture was further stirred at the same temperature for 1.5 hours. To the reaction solution was added water (200 ml), and the solution was extracted with ethyl acetate (200 ml). The organic layer was washed with brine (100 ml), dried over sodium sulfate, and concentrated under reduced pressure. The residue was purified by silica-gel chromatography (column; Hi-Flash™, developing solvent: hexane/ethyl acetate) to give the title compound (5.06 g).

LC-MS, m/z; 307 [M+H]+
(2) (1-{[2-(Trimethylsilyl)ethoxy]methyl}-1H-indazole-3-yl)methanol

Under nitrogen atmosphere, lithium aluminium hydride (1.57 g) was suspended in tetrahydrofuran (70 ml). To the suspension was added dropwise 1-{[2-(trimethylsilyl)ethoxy]methyl}-1H-indazole-3-carboxylic methyl ester (5.06 g) in tetrahydrofuran (30 ml) at −40° C., and the mixture was stirred at the same temperature for 2 hours. To the reaction solution was added sodium fluoride (6.93 g), added dropwise water (2.97 ml), and then added dichloromethane (150 ml). The insoluble residue was removed by Celite filtration, and the filtrate was concentrated under reduced pressure to give the title compound (3.61 g) as an oil.
(3) 3-(Methoxymethyl)-1-{[2-(trimethylsilyl)ethoxy]methyl}-1H-indazole

(1-{[2-(Trimethylsilyl)ethoxy]methyl}-1,4-indazole-3-yl)methanol (2.0 g) was dissolved in tetrahydrofuran (40 ml). To the solution was added sodium hydride (0.53 mg, 55% in silicone oil) at 0° C., and then the mixture was stirred for 1 hour at room temperature. To the reaction solution was added dropwise methyl iodide (805 μl) at 0° C., and the solution was stirred at room temperature overnight. To the reaction solution was added saturated sodium bicarbonate aqueous solution (200 ml). The mixed solution was extracted with ethyl acetate (200 ml). The organic layer was further washed with brine (100 ml), dried over magnesium sulfate, and concentrated under reduced pressure. The residue was purified by silica-gel chromatography (column; Hi-Flash™, developing solvent: hexane/ethyl acetate) to give the title compound (1.35 g).
(4) 3-(Methoxymethyl)-1H-indazole

To 3-(methoxymethyl)-1-{[2-(trimethylsilyl)ethoxy]methyl}-1H-indazole (2.35 g) in tetrahydrofuran (10 ml) were added 1M tetrabutylammonium fluoride/tetrahydrofuran (121 ml) and ethylenediamine (4.05 ml), and the mixture was stirred under reflux for 5 days. The reaction solution was cooled to room temperature, water was added thereto, and the resultant solution was extracted with ethyl acetate (×3). The organic layer was dried over sodium sulfate and concentrated under reduced pressure, and the residue was purified by silica-gel chromatography (column; Hi-Flash™, developing solvent: hexane/ethyl acetate) to give the title compound (0.96 g).

LC-MS, m/z; 163 [M+H]+

### Reference Example 015

### Preparation of 3-(difluoromethyl)-1H-indazole

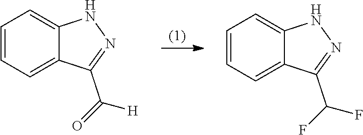 [SMILES: O=Cc1n[nH]c2ccccc12 ; FC(F)c1n[nH]c2ccccc12]

To a mixed solution of Deoxo-Fluor (1.57 ml) and dichloromethane (2.0 ml) were added 1H-indazole-3-carboaldehyde (0.73 g) in dichloromethane (2.0 ml) and ethanol (58 μl) at 0° C., and the solution was stirred for 1 hour at room temperature. To the reaction solution was added saturated sodium bicarbonate aqueous solution (50 ml) at 0° C., and the mixed solution was extracted with ethyl acetate (50 ml) and then further washed with water (50 ml).

The organic layer was dried over sodium sulfate and concentrated under reduced pressure, and then the residue was purified by silica-gel chromatography (column; Hi-Flash™, developing solvent: hexane/ethyl acetate) to give the title compound (0.27 g).

LC-MS, m/z; 167 [M-E]

### Reference Example 016

### Preparation of 3-ethyl-6-fluoro-N′-hydroxy-1H-indazole-1-carboximidamide

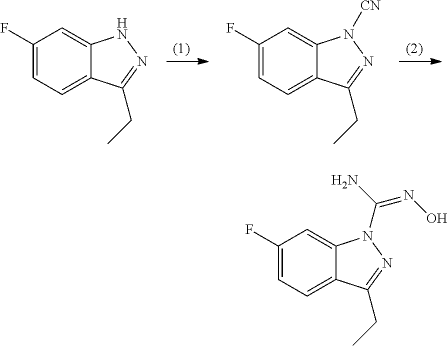

(1) 3-Ethyl-6-fluoro-1H-indazole (0.95 g) was dissolved in dichloromethane (15 ml). To the solution were added triethylamine (1.21 ml), N,N-dimethyl-4-aminopyridine (170 mg) and cyanogen bromide (674 mg), and the mixture was stirred at room temperature for 3.5 hours. The reaction solution was concentrated under reduced pressure, and the resultant crude-product was used for the next reaction.

(2) The above crude-product was suspended in a mixed solvent of THF/water (10/1) (15 ml). To the suspension were added hydroxylamine hydrochloride (523 mg) and triethylamine (1.61 ml), and the mixture was stirred for 1.5 hours with heating at 60° C. and then cooled to room temperature. To the reaction solution was added water (50 ml), and the mixture was extracted with ethyl acetate (50 ml) and then washed with brine (50 ml). The organic layer was dried over sodium sulfate and concentrated under reduced pressure to give the crude product of the title compound (1.29 g).

LC-MS, m/z; 223 [M+H]+

The compounds in the following table (i.e. Reference Examples 017 to 032) were prepared in the same manner as in Reference Example 016 except that the 3-ethyl-6-fluoro-1H-indazole was replaced with the corresponding starting compound (which is commercially available or described in Reference Examples 001 to 015).

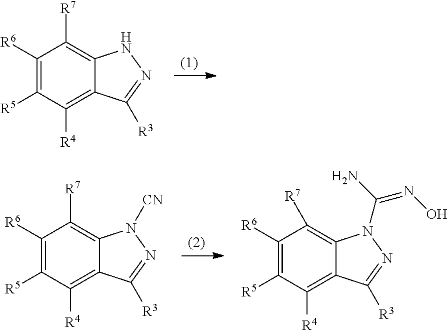

TABLE 2

| Ref. | | | | | | Compound | ¹H-NMR/LC-MS, |
|---|---|---|---|---|---|---|---|
| Ex. | R³ | R⁴ | R⁵ | R⁶ | R⁷ | Name | m/z |
| 017 | ⁱPr | H | H | H | H | N′-hydroxy-3- | LC-MS, m/z; |
| | | | | | | (propan-2-yl)- | 219 [M + H]+ |
| | | | | | | 1H-indazole-1- | |
| | | | | | | carboximidamide | |
| 018 | Et | H | H | H | H | 3-ethyl-N′- | |
| | | | | | | hydroxy-1H- | LC-MS, m/z; |
| | | | | | | indazole-1- | 205 [M + H]+ |
| | | | | | H | carboximidamide | |
| 019 | 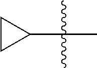 [SMILES: *C1CC1] | H | H | H | | 3-cyclopropyl- N′-hydroxy-1H- indazole-1- carboximidamide | LC-MS, m/z; 217 [M + H]+ |
| 020 | Et | F | H | H | H | 3-ethyl-4- | LC-MS, m/z; |
| | | | | | | fluoro-N′- | 223 [M + H]+ |
| | | | | | | hydroxy-1H- | |
| | | | | | | indazole-1- | |
| | | | | | | carboximidamide | |
| 021 | Et | H | F | H | H | 3-ethyl-5- | LC-MS, m/z; |
| | | | | | | fluoro-N′- | 223 [M + H]+ |
| | | | | | | hydroxy-1H- | |
| | | | | | | indazole-1- | |
| | | | | | | carboximidamide | |
| 022 | Et | H | H | H | F | 3-ethyl-7- | LC-MS, m/z; |
| | | | | | | fluoro-N′- | 223 [M + H]+ |
| | | | | | | hydroxy-1H- | |
| | | | | | | indazole-1- | |
| | | | | | | carboximidamide | |
| 023 | 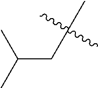 [SMILES: *CC(C)C] | H | H | H | H | N′-hydroxy-3-(2- methylpropyl)- 1H-indazole-1- carboximidamide | LC-MS, m/z; 233 [M + H]+ |
| 024 | Me | H | H | H | H | N′-hydroxy-3- | LC-MS, m/z; |
| | | | | | | methyl-1H- | 191 [M + H]+ |
| | | | | | | indazole-1- | |
| | | | | | | carboximidamide | |
| 025 | 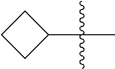 [SMILES: *C1CCC1] | H | H | H | H | 3-cyclobutyl-N′- hydroxy-1H- indazole-1- carboximidamide | ^{l}H-NMR (400 MHz, CDCl₃): (δ 1.88-2.01 (m, 1H), 2.02-2.18 (m, 1H), 2.36- 2.51 (m, 4H), 3.80-3.91 (m, 1H), 5.57 (s, 1H), 7.06 (t, J = 7.5 Hz, 1H), 7.38 (t, J = 7.6 Hz, 1H), 7.60 (d, J = 8.0 Hz, 1H), 8.13 (d, J = 8.4 Hz, 1H). |
| 026 | ⁱPr | H | H | H | F | 7-fluoro-N′- | LC-MS, m/z; |
| | | | | | | hydroxy-3- | 237 [M + H]+ |
| | | | | | | (propan-2-yl)- | |
| | | | | | | 1H-indazole-1- | |
| | | | | | | carboximidamide | |
| 027 | Et | H | H | MeO | H | 3-ethyl-N′- | LC-MS, m/z; |
| | | | | | | hydroxy-6- | 235 [M + H]+ |
| | | | | | | methoxy-1H- | |
| | | | | | | indazole-1- | |
| | | | | | | carboximidamide | |
| 028 | Et | H | H | H | Cl | 7-chloro-3- | LC-MS, m/z; |
| | | | | | | ethyl-N′- | 239 [M + H]+ |
| | | | | | | hydroxy-1H- | |
| | | | | | | indazole-1- | |
| | | | | | | carboximidamide | |
| 029 | 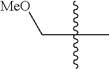 [SMILES: *COC] | H | H | H | H | N′-hydroxy-3- (methoxymethyl)- 1H-indazole-1- carboximidamide | LC-MS, m/z; 221 [M + H]+ |
| 030 | 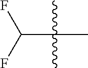 [SMILES: *C(F)F] | H | H | H | H | 3- (difluoromethyl)- N′-hydroxy-1H- indazole-1- carboximidamide | LC-MS, m/z; 227 [M + H]+ |
| 031 | Cl | H | H | H | H | 3-chloro-N′- | LC-MS, m/z; |
| | | | | | | hydroxy-1H- | 211 [M + H]+ |
| | | | | | | indazole-1- | |
| | | | | | | carboximidamide | |
| 032 | Br | H | H | H | H | 3-bromo-N′- | LC-MS, m/z; |
| | | | | | | hydroxy-1H- | 255 [M + H]+ |
| | | | | | | indazole-1- | |
| | | | | | | carboximidamide | |

### Reference Example 033

### Preparation of tert-butyl 4-[3-(3-ethyl-6-fluoro-1H-indazol-1-yl)-1,2,4-oxadiazol-5-yl]piperidine-1-carboxylate

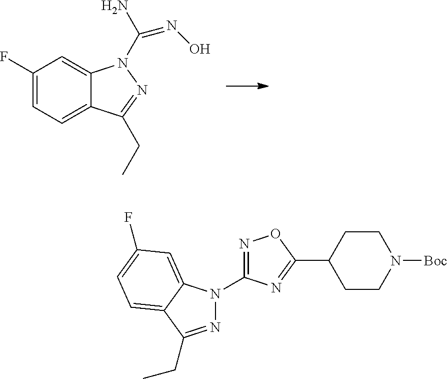

1-(Tert-butoxycarbonyl)piperidine-4-carboxylic acid (1.46 g) was dissolved in THF (10 ml). To the solution was added N,N′-carbonylimidazole (1.03 g), and the mixed solution was stirred at room temperature for 1 hour. To the reaction solution was added dropwise 3-ethyl-6-fluoro-N′-hydroxy-1H-indazole-1-carboximidamide (1.29 g) in THF (10 ml), and the mixture was stirred at room temperature overnight. To the mixture was added 1M tetrabutylammonium fluoride in THF (6.95 ml), and the mixture was stirred at 50° C. for 1.5 hours. The reaction solution was concentrated under reduced pressure, and the residue was purified by silica-gel chromatography (column; Hi-Flash™, developing solvent: hexane/ethyl acetate) to give the title compound (1.66 g).

LC-MS, m/z; 460 [M+HCOO]−

The compounds in the following table (i.e. Reference Examples 034 to 043) were prepared in the same manner as in Reference Example 033 except that the 3-ethyl-6-fluoro-N′-hydroxy-1,4-indazole-1-carboximidamide was replaced with the corresponding starting compound (which is described in Reference Examples 016 to 032).

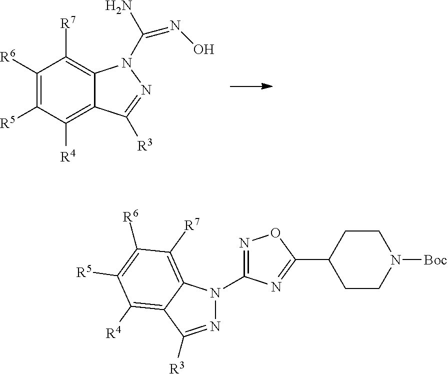

TABLE 3

| Ref. | | | | | | Compound | |
|---|---|---|---|---|---|---|---|
| Ex. | R³ | R⁴ | R⁵ | R⁶ | R⁷ | Name | LC-MS, m/z |
| 034 | Et | F | H | H | H | tert-butyl 4-[3- | LC-MS, m/z; |
| | | | | | | (3-ethyl-4- | 460 [M + HCOO]− |
| | | | | | | fluoro-1H- | |
| | | | | | | indazol-1-yl)- | |
| | | | | | | 1,2,4-oxadiazol- | |
| | | | | | | 5-yl]piperidine- | |
| | | | | | | 1-carboxylate | |
| 035 | Et | H | F | H | H | tert-butyl 4-[3- | LC-MS, m/z; |
| | | | | | | (3-ethyl-5- | 460 [M + HCOO]− |
| | | | | | | fluoro-1H- | |
| | | | | | | indazol-1-yl)- | |
| | | | | | | 1,2,4-oxadiazol- | |
| | | | | | | 5-yl]piperidine- | |
| | | | | | | 1-carboxylate | |
| 036 | Et | H | H | H | F | tert-butyl 4-[3- | LC-MS, m/z; |
| | | | | | | (3-ethyl-7- | 460 [M + HCOO]− |
| | | | | | | fluoro-1H- | |
| | | | | | | indazol-1-yl)- | |
| | | | | | | 1,2,4-oxadiazol- | |
| | | | | | | 5-yl]piperidine- | |
| | | | | | | 1-carboxylate | |
| 037 | 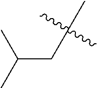 [SMILES: *CC(C)C] | H | H | H | H | tert-butyl 4-{3- [3-(2- methylpropyl)-1H- indazol-1-yl]- 1,2,4-oxadiazol- 5-yl}piperidine- 1-carboxylate | LC-MS, m/z; 470 [M + HCOO]− |
| 038 | ⁱPr | H | H | H | F | tert-butyl 4-{3- | LC-MS, m/z; |
| | | | | | | [7-fluoro-3- | 474 [M + HCOO]− |
| | | | | | | (propan-2-yl)-1H- | |
| | | | | | | indazol-1-yl]- | |
| | | | | | | 1,2,4-oxadiazol- | |
| | | | | | | 5-yl}piperidine- | |
| | | | | | | 1-carboxylate | |
| 039 | Et | H | H | MeO | H | tert-butyl 4-[3- | LC-MS, m/z; |
| | | | | | | (3-ethyl-6- | 472 [M + HCOO]− |
| | | | | | | methoxy-1H- | |
| | | | | | | indazol-1-yl)- | |
| | | | | | | 1,2,4-oxadiazol- | |
| | | | | | | 5-yl]piperidine- | |
| | | | | | | 1-carboxylate | |
| 040 | Et | H | H | H | Cl | tert-butyl 4-[3- | LC-MS, m/z; |
| | | | | | | (7-chloro-3- | 432 [M + H]+ |
| | | | | | | ethyl-1H-indazol- | |
| | | | | | | 1-yl)-1,2,4- | |
| | | | | | | oxadiazo1-5- | |
| | | | | | | yl]piperidine-1- | |
| | | | | | | carboxylate | |
| 041 | 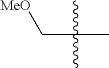 [SMILES: *COC] | H | H | H | H | tert-butyl 4-{3- [3- (methoxymethyl)- 1H-indazol-1-yl]- 1,2,4-oxadiazol- 5-yl}piperidine- 1-carboxylate | No data |
| 042 | 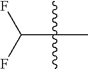 [SMILES: *C(F)F] | H | H | H | H | tert-butyl 4-{3- [3- (difluoromethyl)- 1H-indazol-1-yl]- 1,2,4-oxadiazol- 5-yl}piperidine- 1-carboxylate | LC-MS, m/z; 420 [M + H]+ |
| 043 | Br | H | H | H | H | tert-butyl 4-[3- | LC-MS, m/z; |
| | | | | | | (3-bromo-1H- | 448 [M + H]+ |
| | | | | | | indazol-1-yl)- | |
| | | | | | | 1,2,4-oxadiazol- | |
| | | | | | | 5-yl]piperidine- | |
| | | | | | | 1-carboxylate | |

### Reference Example 044

### Preparation of tert-butyl 4-{3-[3-(propan-2-yl)-1H-indazol-1-yl]-1,2,4-oxadiazol-5-yl}piperidine-1-carboxylate

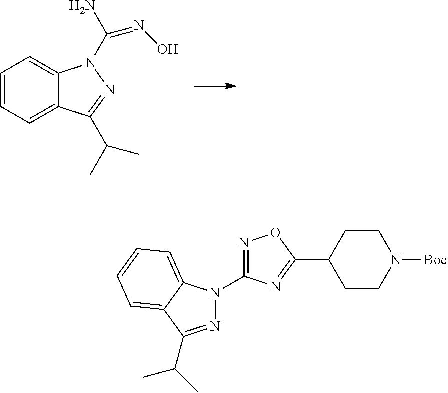 [SMILES: CC(C)c1nn(/C(N)=N\O)c2ccccc12 ; CC(C)c1nn(-c2noc(C3CCN(C(=O)OC(C)(C)C)CC3)n2)c2ccccc12]

1-(Tert-butoxycarbonyl)piperidine-4-carboxylic acid (0.54 g) was dissolved in DMF (5 ml). To the solution was added N,N′-carbonylimidazole (0.38 g), and the mixed solution was stirred at room temperature for 2 hours. To the reaction solution was added N′-hydroxy-3-(propan-2-yl)-1H-indazole-1-carboximidamide (0.49 g), and the mixed solution was stirred at 110° C. for 20 hours and then cooled to room temperature. To the reaction solution was added water, and the mixture was extracted with ethyl acetate. The organic layer was washed with water and brine, dried over sodium sulfate and filtered, and the filtrate was concentrated under reduced pressure. The residue was purified by silica-gel chromatography (developing solvent: hexane/ethyl acetate=6/1) to give the title compound (0.64 g).

LC-MS, m/z; 412 [M+H]+

The compounds in the following table (i.e. Reference Examples 045 to 049) were prepared in the same manner as in Reference Example 044 except that the NT-hydroxy-3-(propan-2-yl)-1H-indazole-1-carboximidamide was replaced with the corresponding starting compound (which is described in Reference Examples 016 to 032).

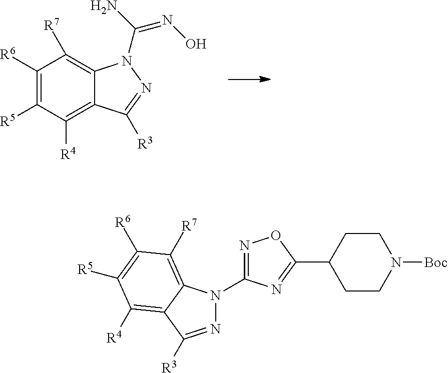

TABLE 4

| | | | | | | Com- | |
|---|---|---|---|---|---|---|---|
| Ref. | | | | | | pound | LC-MS, |
| Ex. | R³ | R⁴ | R⁵ | R⁶ | R⁷ | Name | m/z |
| 045 | Et | H | H | H | H | tert-butyl 4- | LC-MS, |
| | | | | | | [3- | m/z; |
| | | | | | | (3-ethyl-1H- | 398 |
| | | | | | | indazol-1- | [M + H]+ |
| | | | | | | yl)-1,2,4- | |
| | | | | | | oxadiazol- | |
| | | | | | | 5- | |
| | | | | | | yl]piperidine- | |
| | | | | | | 1-carboxylate | |
| 046 | 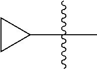 [SMILES: *C1CC1] | H | H | H | H | tert-butyl 4- [3-(3- cyclopropyl- 1H-indazol- 1-yl)-1,2,4- oxadiazol- 5-yl] piperidine- 1-carboxylate | LC-MS, m/z; 432 [M + Na]+ sodium adduct |
| 047 | Me | H | H | H | H | tert-butyl 4- | LC-MS, |
| | | | | | | [3- | m/z; |
| | | | | | | (3-methyl- | 384 |
| | | | | | | 1H- | [M + H]+ |
| | | | | | | indazol-1- | |
| | | | | | | yl)- | |
| | | | | | | 1,2,4- | |
| | | | | | | oxadiazol- | |
| | | | | | | 5- | |
| | | | | | | yl]piperidine- | |
| | | | | | | 1-carboxylate | |
| 048 | 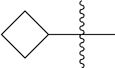 [SMILES: *C1CCC1] | H | H | H | H | tert-butyl 4- [3-(3- cyclobutyl- 1H- indazol-1- yl)-1,2,4- oxadiazol- 5- yl]piperidine- 1-carboxylate | LC-MS, m/z; 446 [M + Na]+ sodium adduct |
| 049 | Cl | H | H | H | H | tert-butyl 4- | LC-MS, |
| | | | | | | [3- | m/z; |
| | | | | | | (3-chloro- | 404 |
| | | | | | | 1H- | [M + H]+ |
| | | | | | | indazol-1- | |
| | | | | | | yl)- | |
| | | | | | | 1,2,4- | |
| | | | | | | oxadiazol- | |
| | | | | | | 5-yl] | |
| | | | | | | piperidine- | |
| | | | | | | 1-carboxylate | |

The compounds in the following table (i.e. Reference Examples 050 to 052) were prepared in the same manner as in Reference Example 044 except that the N′-hydroxy-3-(propan-2-yl)-1H-indazole-1-carboximidamide and the 1-(tert-butoxycarbonyl)piperidine-4-carboxylic acid were replaced with the corresponding starting compound and 1-(tert-butoxycarbonyl)azetidine-3-carboxylic acid, respectively.

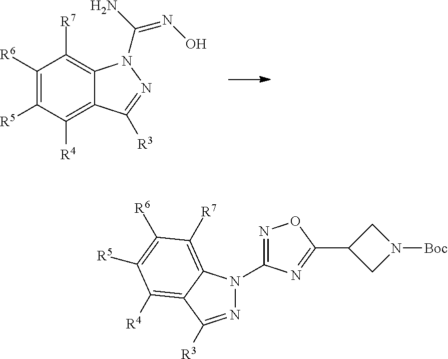

TABLE 5

| Ref. | | | | | | | |
|---|---|---|---|---|---|---|---|
| Ex. | R³ | R⁴ | R⁵ | R⁶ | R⁷ | Compound Name | LC-MS, m/z |
| 050 | Et | H | H | H | H | tert-butyl 3-[3- | LC-MS, m/z; |
| | | | | | | (3-ethyl-1H- | 313 [M − tBu + H]+ |
| | | | | | | indazol-1-yl)- | des tBu |
| | | | | | | 1,2,4-oxadiazol- | |
| | | | | | | 5-yl]azetidine-1- | |
| | | | | | | carboxylate | |
| 051 | ⁱPr | H | H | H | H | tert-butyl 3-{3- | LC-MS, m/z; |
| | | | | | | [3-(propan-2-yl)- | 344 [M − tBu + |
| | | | | | | 1H-indazol-1-yl]- | NH4]+ des tBu, |
| | | | | | | 1,2,4-oxadiazol- | ammonia |
| | | | | | | 5-yl}azetidine-1- | adduct |
| | | | | | | carboxylate | |
| 052 | Et | H | H | F | H | tert-butyl 3-[3- | LC-MS, m/z; |
| | | | | | | (3-ethyl-6- | 331 [M − tBu + H]+ |
| | | | | | | fluoro-1H- | |
| | | | | | | indazol-1-yl)- | |
| | | | | | | 1,2,4-oxadiazol- | |
| | | | | | | 5-yl]azetidine-1- | |
| | | | | | | carboxylate | |

### Reference Example 053

### Preparation of tert-butyl 4-(2-iodoethyl)piperidine-1-carboxylate

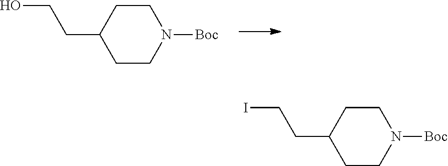 [SMILES: CC(C)(C)OC(=O)N1CCC(CCO)CC1 ; CC(C)(C)OC(=O)N1CCC(CCI)CC1]

Tert-butyl 4-(2-hydroxyethyl)piperidine-1-carboxylate (2.29 g) was dissolved in methylene chloride (40 ml). To the solution were added iodine (3.05 g), triphenylphosphine (3.41 g) and imidazole (1.02 g), and the mixture was stirred at room temperature overnight. The reaction solution was concentrated under reduced pressure. To the residue were added methylene chloride (3 ml) and diethyl ether (3 ml), and the precipitated insoluble-matter was removed by filtration. The filtrate was concentrated under reduced pressure, and the residue was purified by silica-gel chromatography (developing solvent: hexane/ethyl acetate=5/1) to give tert-butyl 4-(2-iodoethyl)piperidine-1-carboxylate (3.00 g) as a colorless oil.

LC-MS, m/z; 340 [M+H]+

### Reference Example 054

### Preparation of tert-butyl 3-({[(4-methylphenyl)sulfonyl]oxy}methyl)piperidine-1-carboxylate

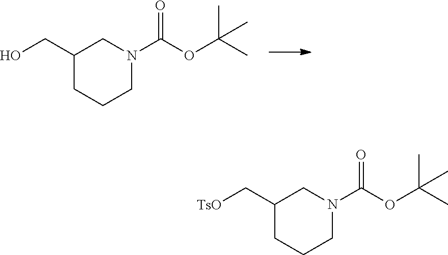 [SMILES: CC(C)(C)OC(=O)N1CCCC(CO)C1 ; Cc1ccc(S(=O)(=O)OCC2CCCN(C(=O)OC(C)(C)C)C2)cc1]

Tert-butyl 3-(hydroxymethyl)piperidine-1-carboxylate (166 g) was dissolved in toluene (1.2 L). To the solution were added trimethylamine hydrochloride (7.37 g) and triethylamine (161 ml). To the mixture was slowly added 4-methylbenzenesulfonyl chloride (176 g) at 0° C. with stirring, and then the resultant mixture was stirred at room temperature for 6 hours. The reaction solution was washed sequentially with 30% citric acid aqueous solution, water, and brine. The organic layer was dried over sodium sulfate and filtered, and the filtrate was concentrated under reduced pressure. To the residue were added tert-butyl methyl ether (5 ml) and hexane (800 ml), and the mixture was stirred at room temperature for 2 hours. The resulting crystal was collected on a filter to give tert-butyl 3-({[(4-methylphenyl)sulfonyl]oxy}methyl)piperidine-1-carboxylate (234.5 g) as a white solid.

LC-MS, m/z; 370 [M+H]+.

### Reference Example 055

### Preparation of tert-butyl (3S)-3-(iodomethyl)pyrrolidine-1-carboxylate

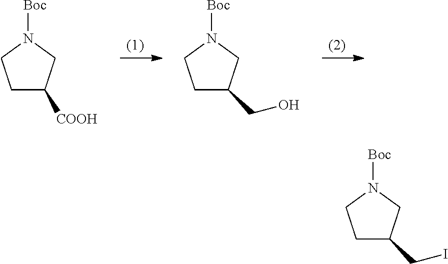 [SMILES: CC(C)(C)OC(=O)N1CC[C@H](C(=O)O)C1 ; CC(C)(C)OC(=O)N1CC[C@H](CO)C1 ; CC(C)(C)OC(=O)N1CC[C@H](CI)C1]

(1) (3S)-1-(Tert-butoxycarbonyl)pyrrolidine-3-carboxylic acid (10 g) was dissolved in tetrahydrofuran (100 ml). To the solution was added dropwise dimethyl sulfide-borane tetrahydrofuran solution (54 ml) at 0° C. with stirring, and then the mixture was warmed to room temperature and stirred for 3 hours. To the reaction solution was added dropwise methanol (100 ml) at 0° C. with stirring, and then the reaction solution was concentrated under reduced pressure. The residue was purified by silica-gel chromatography (developing solvent: hexane/ethyl acetate=1:9) to give tert-butyl(3S)-3-(hydroxymethyl)pyrrolidine-1-carboxylate (7.8 g) as a colorless oil.

LC-MS, m/z; 202 [M+H]+.

(2) The above-prepared compound (7.8 g) was dissolved in dichloromethane (150 ml). To the solution were added triphenylphosphine (13.3 g), imidazole (3.96 g) and iodine (11.8 g), and the mixture was stirred at 70° C. for 3 hours. To the reaction solution was added saturated sodium thiosulfate aqueous solution. The mixed solution was extracted with chloroform. The organic layer was washed with brine, dried over sodium sulfate and filtered, and the filtrate was concentrated under reduced pressure. The residue was purified by silica-gel chromatography (developing solvent: hexane/ethyl acetate=11:1) to give the title compound (11.5 g) as a white solid.

LC-MS, m/z; 312 [M+H]+

### Reference Example 056

### Preparation of tert-butyl (3R)-3-(iodomethyl)pyrrolidine-1-carboxylate

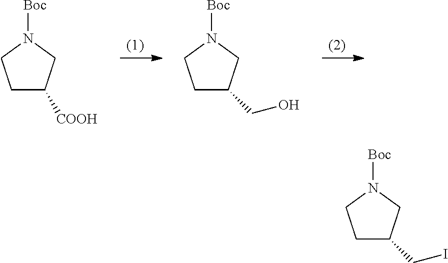 [SMILES: CC(C)(C)OC(=O)N1CC[C@@H](C(=O)O)C1 ; CC(C)(C)OC(=O)N1CC[C@@H](CO)C1 ; CC(C)(C)OC(=O)N1CC[C@@H](CI)C1]

The title compound was prepared in the same manner as in Reference Example 055 except that the (3S)-1-(tert-butoxycarbonyl)pyrrolidine-3-carboxylic acid was replaced with (3R)-1-(tert-butoxycarbonyl)pyrrolidine-3-carboxylic acid.

LC-MS, m/z; 312 [M+H]+

### Reference Example 057

### Preparation of methyl(3-chloropropyl)methylcarbamate

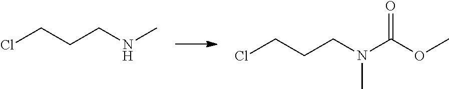

3-Chloro-N-methylpropane-1-amine (0.576 g) was dissolved in dichloromethane (9 ml). To the solution was added triethylamine (1.4 ml) at room temperature with stirring. To the reaction solution was added dropwise methyl chlorocarbonate (0.454 g), and the mixed solution was stirred at room temperature for 4 hours. To the reaction solution was added water. The mixture was extracted with ethyl acetate, the organic layer was washed with brine, dried over sodium sulfate and filtered, and the filtrate was concentrated under reduced pressure to give methyl(3-chloropropyl)methylcarbamate.

LC-MS, m/z; 166 [M+H]+

### Reference Example 058

### Preparation of methyl(2-bromoethyl)carbamate

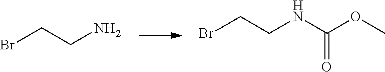

The title compound was prepared in the same manner as in Reference Example 057 except that the 3-chloro-N-methylpropane-1-amine was replaced with 2-bromoethaneamine.

¹H-NMR (400 MHz, CDCl₃): δ 3.45 (t, J=5.6 Hz, 2H), 3.56 (t, J=5.72 Hz, 2H), 3.66 (s, 3H), 5.24 (s, 1H).

### Reference Example 059

### Preparation of 4-[3-(3-ethyl-1H-indazol-1-yl)-1,2,4-oxadiazol-5-yl]cyclohexanone

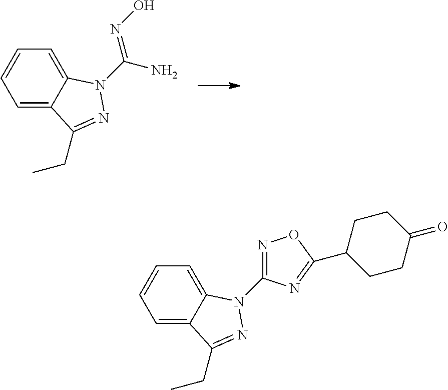 [SMILES: CCc1nn(/C(N)=N/O)c2ccccc12 ; CCc1nn(-c2noc(C3CCC(=O)CC3)n2)c2ccccc12]

The title compound was prepared in the same manner as in Reference Example 033 except that the 1-(tert-butoxycarbonyl)piperidine-4-carboxylic acid and the 3-ethyl-6-fluoro-N′-hydroxy-1H-indazole-1-carboximidamide were replaced with 4-oxocyclohexane carboxylic acid and 3-ethyl-N′-hydroxy-1H-indazole-1-carboximidamide, respectively.

LC-MS, m/z; 311 [M+H]+

### Reference Example 060

### Preparation of 3-[3-(3-ethyl-6-fluoro-1H-indazol-1-yl)-1,2,4-oxadiazol-5-yl]cyclobutanone

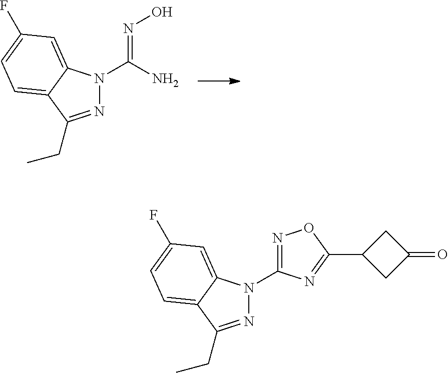 [SMILES: CCc1nn(/C(N)=N/O)c2cc(F)ccc12 ; CCc1nn(-c2noc(C3CC(=O)C3)n2)c2cc(F)ccc12]

3-Oxocyclobutanecarboxylic acid (2.48 g) was dissolved in THF (36 ml). To the solution was added N,N′-carbonylimidazole (3.53 g), and the mixed solution was stirred at room temperature for 1 hour. To the reaction solution was added 3-ethyl-6-fluoro-N-hydroxy-1H-indazole-1-carboximidamide (4.03 g), and the mixed solution was stirred at 50° C. for 4 hours. The reaction solution was cooled to room temperature and then concentrated under reduced pressure, and water was added thereto. The mixture was extracted with chloroform. The organic layer was washed with brine, dried over sodium sulfate and filtered, and the filtrate was concentrated under reduced pressure to give a quantitative amount of 3-ethyl-6-fluoro-N-{[(3-oxocyclobutyl)carbonyl]oxy}-1H-indazole-1-carboximidamide. Then, the 3-ethyl-6-fluoro-N-{[(3-oxocyclobutyl)carbonyl]oxy}-1H-indazole-1-carboximidamide (4.85 g) was dissolved in acetic acid (76 ml), and the solution was stirred at 90° C. for 6 hours. The reaction solution was concentrated under reduced pressure, saturated sodium carbonate aqueous solution was added thereto, and the mixture was extracted with chloroform. The organic layer was washed with brine, dried over sodium sulfate and filtered, and the filtrate was concentrated under reduced pressure. The residue was purified by silica-gel chromatography (developing solvent: hexane/ethyl acetate) to give the title compound (2.69 g).

LC-MS, m/z; 301 [M+H]+

### Reference Example 061

### Preparation of 3-chloro-1-[5-(piperidin-4-yl)-1,3,4-oxadiazol-2-yl]-1H-indazole hydrochloride

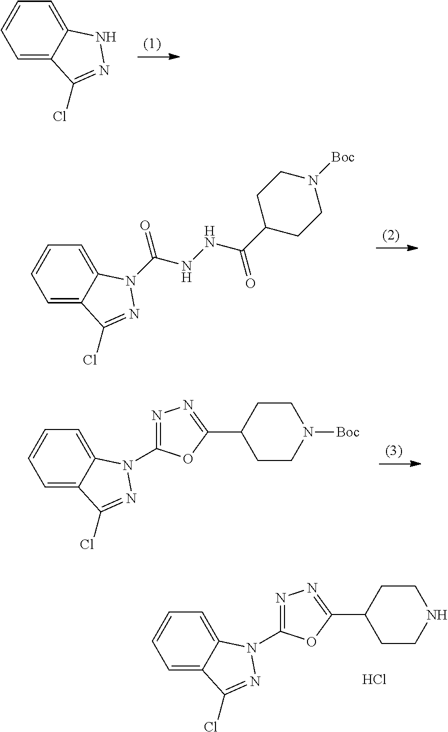 [SMILES: Clc1n[nH]c2ccccc12 ; CC(C)(C)OC(=O)N1CCC(C(=O)NNC(=O)n2nc(Cl)c3ccccc32)CC1 ; CC(C)(C)OC(=O)N1CCC(c2nnc(-n3nc(Cl)c4ccccc43)o2)CC1 ; Clc1nn(-c2nnc(C3CCNCC3)o2)c2ccccc12 ; Cl]

(1) To triphosgene (355 mg) was added methylene chloride (3 ml). To the mixture were added dropwise 3-chloroindazole (458 mg) and triethylamine (1.95 ml) dissolved in methylene chloride (3 ml) with stirring at 0° C. The reaction solution was warmed to room temperature, stirred for 30 minutes, and then cooled to 0° C. again. To the reaction solution were added dropwise tert-butyl 4-(hydrazinecarbonyl)piperidine-1-carboxylate (730 mg) and triethylamine (0.63 ml) dissolved in methylene chloride (3 ml), and the mixture was stirred at room temperature for 4 hours. To the reaction solution was added saturated sodium carbonate aqueous solution, and the resultant solution was extracted with chloroform. The organic layer was dried over sodium sulfate and filtered, and the filtrate was concentrated under reduced pressure. The residue was purified by silica-gel chromatography (developing solvent: chloroform/methanol=95/5) to give tert-butyl 4-({2-[(3-chloro-1H-indazol-1-yl)carbonyl]hydrazinyl}carbonyl)piperidine-1-carboxylate (519 mg).

LC-MS, m/z; 422 [M+H]+

(2) The above-prepared compound (519 mg) was dissolved in methylene chloride (12 ml). To the solution was added triphenylphosphine (645 mg), carbon tetrachloride (0.24 ml) and triethylamine (0.7 ml), and the mixed solution was refluxed overnight. The reaction solution was cooled to room temperature and concentrated under reduced pressure. The residue was purified by silica-gel chromatography (developing solvent: hexane/ethyl acetate

=3/1) to give tert-butyl 4-[5-(3-chloro-1H-indazol-1-yl)-1,3,4-oxadiazol-2-yl]piperidine-1-carboxylate (355 mg) as an oil.

LC-MS, m/z; 404 [M+H]+

(3) To the above-prepared compound (355 mg) was added 4 N HCl/dioxane (10 ml) and methanol (5 ml), and the mixed solution was stirred at room temperature for 5 hours. The reaction solution was concentrated under reduced pressure. To the residue was added ethyl acetate (10 ml), and the crystallized white solid was collected on a filter to give the title compound (229 mg).

LC-MS, m/z; 304 [M+H]+

### Reference Example 062

### Preparation of 1-[3-(piperidin-4-yl)isoxazol-5-yl]-3-(propan-2-yl)-1H-indazole hydrochloride

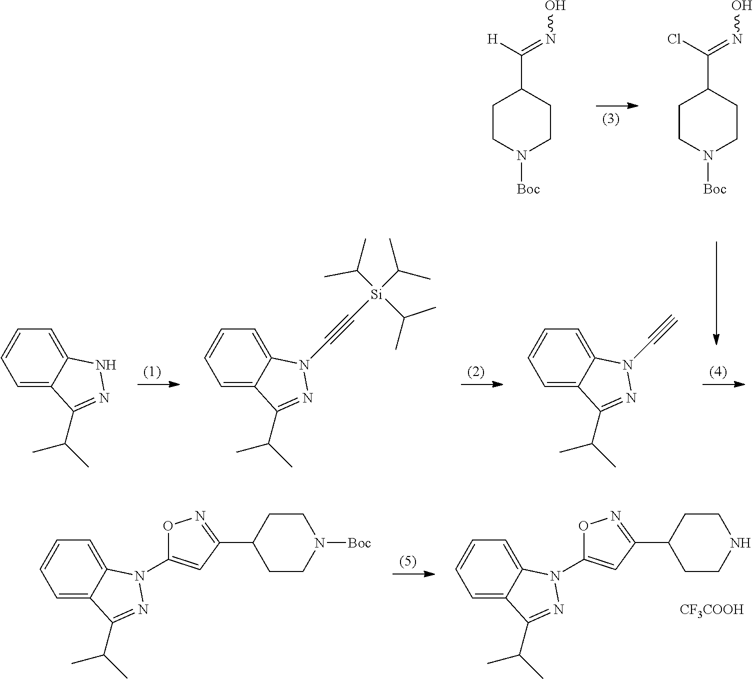

(1) To 3-(propan-2-yl)-1H-indazole (801 mg), copper (II) chloride (297 mg) and sodium carbonate (1.06 g) was added dropwise pyridine (791 mg) in toluene (5 ml) under oxygen atmosphere, and the mixture was stirred at 70° C. for 30 minutes. To the mixture was added dropwise (triisopropylsilyl)acetylene (912 mg) in toluene (5 ml), and the mixture was stirred at 70° C. for 4 hours. The reaction solution was concentrated under reduced pressure, and the residue was purified by silica-gel chromatography (developing solvent: hexane/ethyl acetate=7:3) to give 3-(propan-2-yl)-1-[(tripropan-2-ylsilyl)ethynyl]-1H-indazole (260 mg) as a colorless oil.

(2) The above-prepared compound (260 mg) was dissolved in tetrahydrofuran (14 ml). To the solution was added 1N tetrabutylammonium fluoride/tetrahydrofuran solution (0.9 ml), and the mixed solution was stirred at room temperature for 1 hour. The reaction solution was concentrated under reduced pressure, and the residue was purified by silica-gel chromatography (developing solvent: hexane/ethyl acetate=7:3) to give 1-ethynyl-3-(propan-2-yl)-1H-indazole (82 mg) as a colorless oil.

LC-MS, m/z; 185 [M+H]+

(3) Text-butyl 4-[(hydroxylimino)methyl]piperidine-1-carboxylate (2.59 g) was dissolved in DMF (25 ml). To the solution was added N-chlorosuccinimide (1.47 g), and the mixture was stirred at room temperature for 2 hours and then water (40 ml) was slowly added dropwise thereto with stirring. The crystallized solid was collected on a filter and washed with water. The resultant solid was dried under reduced pressure at 50° C. to give tert-butyl [chloro(hydroxylimino)methyl]piperidine-1-carboxylate (2.31 g) as a white crystal.

(4) 1-Ethynyl-3-(propan-2-yl)-1H-indazole (82 mg), tert-butyl 4-[chloro(hydroxylimino)methyl]piperidine-1-carboxylate (117 mg) and sodium bicarbonate (243 mg) were suspended in toluene (5 ml), and the suspension was stirred at room temperature overnight. To the reaction solution was added water. The resultant solution was extracted with ethyl acetate. The organic layer was washed with brine, dried over sodium sulfate and filtered, and the filtrate was concentrated under reduced pressure. The residue was purified by silica-gel chromatography (developing solvent: hexane/ethyl acetate=7:3) to give tert-butyl 4-{5-[3-(propan-2-yl)-1H-indazol-1-yl]isoxazol-3-yl}piperidine-1-carboxylate (100 mg) as a white solid.

LC-MS, m/z; 411 [M+H]+

(5) Tert-butyl 4-{5-[3-(propan-2-yl)-1H-indazol-1-yl]isoxazol-3-yl}piperidine-1-carboxylate (100 mg) was dissolved in dichloromethane (3 ml). To the solution was added trifluoroacetic acid (3 ml) at 0° C. with stirring, and then the mixture was reacted at room temperature for 3 hours. The reaction solution was concentrated under reduced pressure, toluene (5 ml) was added thereto, and the solution was concentrated under reduced pressure (×3). To the residue was added ethyl acetate to precipitate a crystal, and the resultant was concentrated under reduced pressure to give the title compound (130 mg) as a colorless crystal.

### Reference Example 063

### Preparation of 3-chloro-1-[3-(piperidin-4-yl)-1,2,4-oxadiazol-5-yl]-1H-indazole hydrochloride

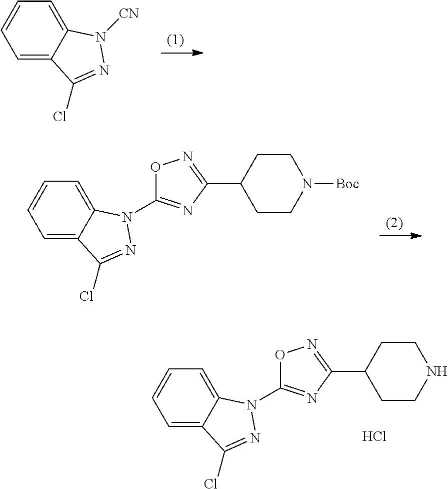 [SMILES: N#Cn1nc(Cl)c2ccccc21 ; CC(C)(C)OC(=O)N1CCC(c2noc(-n3nc(Cl)c4ccccc43)n2)CC1 ; Clc1nn(-c2nc(C3CCNCC3)no2)c2ccccc12 ; Cl]

(1) 3-Chloro-1H-indazole-1-carbonitrile (355 mg), tert-butyl 4-[chloro(hydroxylimino)methyl]piperidine-1-carboxylate (525 mg) and sodium bicarbonate (672 mg) were suspended in toluene (10 ml), and the suspension was stirred at 60° C. overnight. The reaction solution was cooled, and water was added thereto. The mixture was extracted with ethyl acetate. The organic layer was dried over sodium sulfate and filtered, and the filtrate was concentrated under reduced pressure. The residue was purified by silica-gel chromatography (developing solvent: hexane/ethyl acetate=4:1) to give tert-butyl 4-[5-(3-chloro-1H-indazol-1-yl)-1,2,4-oxadiazol-3-yl]piperidine-1-carboxylate (605 mg).

LC-MS, m/z; 404 [M+H]+

(2) To the above-prepared compound (605 mg) were added 4 N HCl/dioxane (15 ml) and methanol (2 ml), and the mixture was stirred at room temperature for 3 hours. The reaction solution was concentrated under reduced pressure, and the crystallized white solid was collected on a filter to give the title compound (360 mg).

LC-MS, m/z; 304 [M+H]+

### Reference Example 064

### Preparation of 1-[5-(piperidin-4-yl)-1,2,4-oxadiazol-3-yl]-1H-pyrrolo[2,3-b]pyridine trifluoroacetate

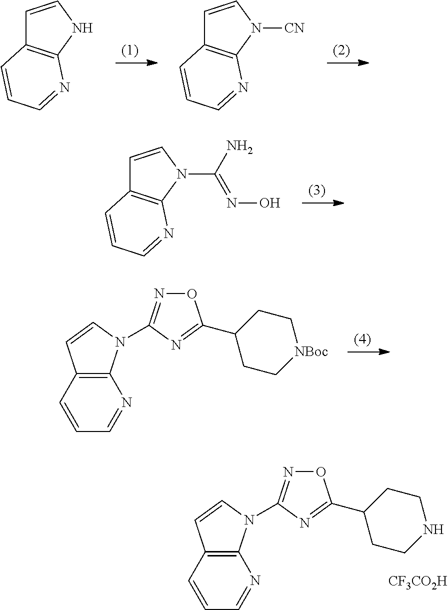

(1) 1H-Pyrrolo[2,3-b]pyridine (1.54 g) was dissolved in dichloromethane (130 ml). To the solution were added triethylamine (3.6 ml), N,N-dimethyl-4-aminopyridine (0.53 g) and cyanogen bromide (4.13 g), and the mixture was stirred at room temperature for 3 hours. To the reaction solution was added water. The resultant solution was extracted with dichloromethane. The organic layer was washed with water and brine, dried over sodium sulfate, and filtered. The filtrate was concentrated under reduced pressure. The residue was purified by silica-gel chromatography (developing solvent: hexane/ethyl acetate) to give 1H-pyrrolo[2,3-b]pyridine-1-carbonitrile (2.12 g).

LC-MS, m/z; 144 [M+H]⁺.

(2) The above-prepared compound (1.98 g) was dissolved in a mixed solvent of ethanol (68 ml) and water (14 ml). To the solution was added hydroxylamine hydrochloride (2.88 g) and potassium carbonate (3.06 g), and the mixture was refluxed. The reaction solution was cooled to room temperature and then concentrated under reduced pressure, water was added thereto, and the mixture was extracted with ethyl acetate. The organic layer was washed with brine, dried over sodium sulfate and filtered, and the filtrate was concentrated under reduced pressure to give N-hydroxy-1,1-pyrrolo[2,3-b]pyridine-1-carboximidamide (1.23 g).

LC-MS, m/z; 177 [M+H]+

(3) The above-prepared compound (1.03 g) was dissolved in DMF (28 ml). To the solution was added 60% sodium hydride (269 mg) at ice temperature, and the mixture was stirred for 1 hour. To the reaction solution was added 1-tert-butyl 4-ethyl piperidine-1,4-dicarboxylate (1.50 g) in DMF (8.5 ml), and the mixture was further stirred at room temperature for 3 hours. To the reaction solution was added water, and the crystallized precipitate was collected on a filter to give tert-butyl 4-[3-(1H-pyrrolo[2,3-b]pyridin-1-yl)-1,2,4-oxadiazol-5-yl]piperidine-1-carboxylate (1.10 g).

LC-MS, m/z; 370 [M+H]⁺.

(4) The above-prepared compound (1.10 g) was dissolved in dichloromethane (22 ml). To the solution was added trifluoroacetic acid (2.2 ml), and the mixed solution was stirred at room temperature overnight. The reaction solution was concentrated under reduced pressure to give a quantitative amount of the title compound.

LC-MS, m/z; 270 [M+H]+

### Reference Example 065

### Preparation of 1-[5-(piperidin-4-yl)-1,2,4-oxadiazol-3-yl]-6-(propan-2-yl)-1H-pyrrolo[2,3-b]pyridine hydrochloride

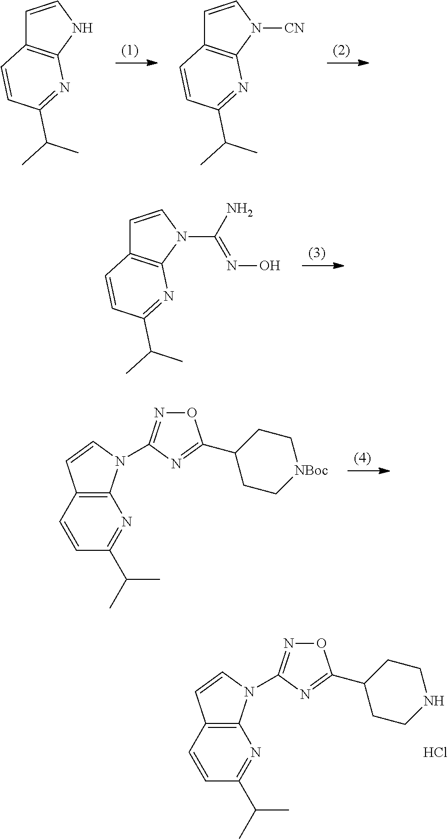 [SMILES: CC(C)c1ccc2cc[nH]c2n1 ; CC(C)c1ccc2ccn(C#N)c2n1 ; CC(C)c1ccc2ccn(/C(N)=N/O)c2n1 ; CC(C)c1ccc2ccn(-c3noc(C4CCN(C(=O)OC(C)(C)C)CC4)n3)c2n1 ; CC(C)c1ccc2ccn(-c3noc(C4CCNCC4)n3)c2n1 ; Cl]

(1) 6-Isopropyl-1H-pyrrolo[2,3-b]pyridine (239 mg) was dissolved in dichloromethane (15 ml). To the solution were added triethylamine (0.42 ml), N,N-dimethyl-4-aminopyridine (61 mg) and cyanogen bromide (474 mg), and the mixture was stirred at room temperature for 24 hours. To the reaction solution was added water, and the resultant solution was extracted with dichloromethane. The organic layer was washed with water and brine, dried over sodium sulfate and filtered, and the filtrate was concentrated under reduced pressure. The residue was purified by silica-gel chromatography (developing solvent: hexane ethyl acetate) to give 6-(propan-2-yl)-1H-pyrrolo[2,3-b]pyridine-1-carbonitrile (128 mg).

LC-MS, m/z; 186 [M+H]+

(2) The above-prepared compound (128 mg) was dissolved in a mixed solvent of THF (3.5 ml) and water (0.35 ml). To the solution were added hydroxylamine hydrochloride (62 mg) and triethylamine (0.19 ml), and the mixture was refluxed for 2 hours. The reaction solution was cooled to room temperature, water was added thereto, and the resultant solution was extracted with ethyl acetate. The organic layer was washed with brine, dried over sodium sulfate and filtered, and the filtrate was concentrated under reduced pressure to give a quantitative amount of N-hydroxy-6-(propan-2-yl)-1H-pyrrolo[2,3-b]pyridine-1-carboximidamide.

LC-MS, m/z; 219 [M+H]+

(3) 1-(Tert-butoxycarbonyl)piperidine-4-carboxylic acid (181 mg) was dissolved in DMF (1.4 ml). To the solution was added N,N′-carbonylimidazole (128 mg), and the mixture was stirred at room temperature for 1 hour. To the reaction solution was added the above-prepared compound (157 mg) in DMF (1.4 ml), and the mixture was stirred at 120° C. for 12 hours. The reaction solution was cooled to room temperature, water was added thereto, and the resultant solution was extracted with ethyl acetate. The organic layer was washed with water several times, dried over sodium sulfate and filtered, and the filtrate was concentrated under reduced pressure to give tert-butyl 4-{3-[6-(propan-2-yl)-1H-pyrrolo[2,3-b]pyridin-1-yl]-1,2,4-oxadiazol-5-yl}piperidine-1-carboxylate (278 mg).

LC-MS, m/z; 412 [M+H]+

(4) To the above-prepared compound (278 mg) was added 4 NHCl/1,4-dioxane (14 ml), and the mixture was stirred at room temperature for 4 hours. The reaction solution was concentrated under reduced pressure to give a quantitative amount of the title compound.

LC-MS, m/z; 312 [M+H]+

### Reference Example 066

### Preparation of 2-(tetrahydrofuran-2-yl)ethanol

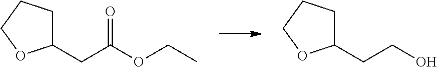

Lithium aluminum hydride (4.20 g) was stirred in THF (100 ml) under nitrogen atmosphere at −40° C. To the mixture was slowly added dropwise ethyl tetrahydrofuran-2-acetate (7.0 g) in THF (63 ml). After the dropping, the mixture was stirred at −40° C. for 1.5 hours. After confirming the completion of the reaction, sodium fluoride (18.6 g) and water (8.0 ml) were added thereto, and the mixture was stirred. The reaction solution was filtered through Celite, and the resultant solution was evaporated under reduced pressure to give 2-(tetrahydrofuran-2-yl) ethanol (4.40 g) as a colorless oil.

LC-MS, m/z; 117 [M+H]+

### Reference Example 067

### Preparation of 2-(tetrahydrofuran-2-yl)ethyl 4 methylbenzenesulfonate

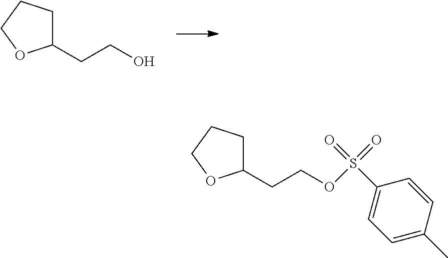 [SMILES: OCCC1CCCO1 ; Cc1ccc(S(=O)(=O)OCCC2CCCO2)cc1]

To 2-(tetrahydrofuran-2-yl)ethanol (4.40 g) in dichloromethane (160 ml) were added triethylamine (10.6 ml), trimethylamine hydrochloride (0.362 g) and p-toluenesulfonic acid chloride (7.94 g), and the mixture was stirred at 0° C. After the reaction was completed, water was added to the reaction solution. The mixture was extracted with chloroform, the organic layer was dried over sodium sulfate and filtered, and the filtrate was concentrated under reduced pressure to give 2-(tetrahydrofuran-2-yl)ethyl 4-methylbenzenesulfonate (10.19 g) as a colorless oil.

LC-MS, m/z; 271 [M+H]+

### Reference Example 068

### Preparation of 2-(tetrahydropyran-2-yl)ethyl 4-methylbenzenesulfonate

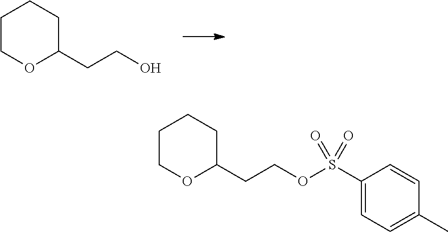 [SMILES: OCCC1CCCCO1 ; Cc1ccc(S(=O)(=O)OCCC2CCCCO2)cc1]

2-(2-Hydroxyethyl)-tetrahydropyrane (0.50 g) was reacted with p-toluenesulfonic acid chloride (0.805 g) in the same manner as in Reference Example 067 to give 2-(tetrahydropyran-2-yl)ethyl 4-methylbenzenesulfonate (1.00 g) as a colorless oil.

LC-MS, m/z; 285 [M+H]+

### Reference Example 069

### Preparation of (tetrahydro-2H-pyran-3-yl)methyl 4-methylbenzenesulfonate

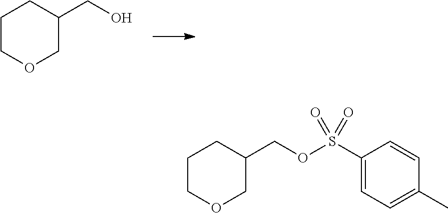 [SMILES: OCC1CCCOC1 ; Cc1ccc(S(=O)(=O)OCC2CCCOC2)cc1]

(Tetrahydro-2H-pyran-3-yl)methanol (0.45 g) was reacted with p-toluenesulfonic acid chloride (0.812 g) in the same manner as in Reference Example 067 to give (tetrahydro-2H-pyran-3-yl)methyl 4-methylbenzenesulfonate (1.21 g) as a colorless oil.

LC-MS, m/z; 271 [M+H]+

### Reference Example 070

### Preparation of 2-(7-fluoro-1H-indazol-3-yl)propan-2-ol

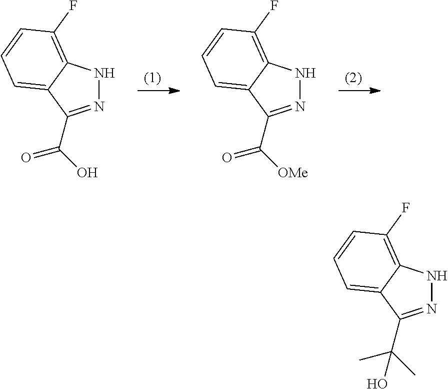

(1) 7-Fluoro-1H-indazole-3-carboxylic acid (8.0 g) was dissolved in methanol (500 ml). To the solution was added concentrated H₂SO₄ (15 ml) at ice temperature, and the mixed solution was stirred under reflux for 7 hours. The solvent was removed under reduced pressure, chloroform was added to the residue, and the resultant was neutralized with saturated sodium bicarbonate aqueous solution. The organic layer was further washed with water, dried, and concentrated under reduced pressure. The residue was purified by silica-gel chromatography (column; Hi-Flash™ developing solvent: hexane/ethyl acetate) and then purified again by silica-gel chromatography (column; Hi-Flash™, developing solvent: chloroform/methanol) to give methyl 7-fluoro-1H-indazole-3-carboxylate (4.15 g) as a white crystal.

(2) Methyl 7-fluoro-1H-indazole-3-carboxylate (2.4 g) was dissolved in THF (70 ml), and the solution was cooled to −70° C. To the solution was added dropwise CH₃MgI/diethyl ether (2.0 M, 21.63 ml) under nitrogen atmosphere.

The reaction solution was stirred overnight with heating at 50° C. After the reaction was completed, saturated ammonium chloride aqueous solution (100 ml) was added dropwise to the mixture at ice temperature. The mixture was extracted with ethyl acetate, the organic layer was further washed with brine, dried and concentrated under reduced pressure, and the residue was purified by silica-gel chromatography (column; Hi-Flash™, developing solvent: hexane/ethyl acetate) to give the title compound (2.06 g) as a white crystal.

LC-MS, m/z; 195 [M+H]+

### Reference Example 071

### Preparation of 2-fluoro-3-methoxyaniline hydrochloride

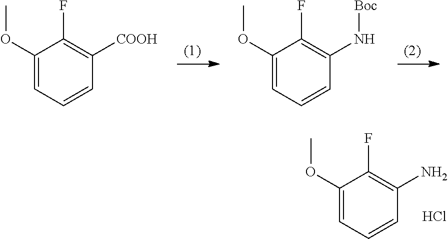

(1) 2-Fluoro-3-methoxybenzoic acid (5.1 g), triethylamine (5.06 ml) and diphenylphosphoryl azide (9.08 g) were added to tert-butyl alcohol (100 ml), and the mixture was refluxed overnight. Then, the reaction solution was cooled and concentrated under reduced pressure, and the residue was purified by silica-gel chromatography (column; Hi-Flash™, developing solvent: hexane/ethyl acetate 10:1) to give tert-butyl (2-fluoro-3-methoxyphenyl)carbamate (5.28 g) as a colorless solid.

(2) Tert-butyl (2-fluoro-3-methoxyphenyl)carbamate (5.28 g) was dissolved in 4N HCl/dioxane (30 ml), and the solution was stirred at room temperature for 3 hours. Then, the reaction solution was evaporated under reduced pressure, toluene (100 ml) was added thereto, the mixture was evaporated again under reduced pressure, and the residue was dried under reduced pressure to give the title compound (3.89 g) as a white solid.

LC-MS, m/z; 142 [M+H]+

### Reference Example 072

### Preparation of 2-fluoro-5-methoxyaniline hydrochloride

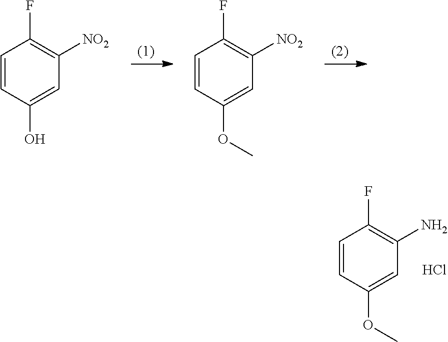

(1) 4-Fluoro-3-nitrophenol (3.14 g) was dissolved in acetone (40 ml). To the solution were added methyl iodide (5.68 g) and potassium carbonate (5.53 g), and the mixture was stirred at 40° C. for 6 hours. Then, methylene chloride (50 ml) was added thereto, the insoluble matter was removed by filtration, and the filtrate was concentrated under reduced pressure. The residue was dissolved in ethyl acetate (50 ml). The organic layer was washed with 1N sodium hydroxide aqueous solution, water and brine, dried over sodium sulfate, and filtered. The filtrate was concentrated under reduced pressure to give 1-fluoro-4-methoxy-2-nitrobenzene (3.47 g) as a brown oil.

(2) 1-Fluoro-4-methoxy-2-nitrobenzene (3.47 g) was dissolved in methanol (30 ml). To the solution was added 10% palladium/carbon (2 g), and the mixed solution was stirred under hydrogen atmosphere for 5 hours. The reaction solution was filtered through Celite, and the filtrate was concentrated under reduced pressure. The residue was dissolved in ethyl acetate (10 ml), and 4 N HCl/ethyl acetate solution was added dropwise thereto. The resulting crystal was collected on a filter, and dried to give the title compound (3.2 g) as a brown solid.

LC-MS, m/z; 142 [M+H]+

The compounds in the following table (i.e. Reference Examples 073 to 075) were prepared in the same manner as in Reference Example 013 except that the aniline and cyclopropylcyanide of (1) in Reference Example 013 were replaced with the corresponding starting compound and isopropylcyanide, respectively.

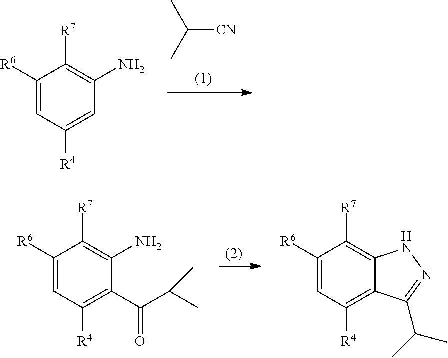

TABLE 6

| Ref. | | | | Compound | |
|---|---|---|---|---|---|
| Ex. | R⁴ | R⁶ | R⁷ | Name | LC-MS, m/z |
| 073 | H | MeO | F | 7-fluoro-6- | LC-MS, m/z; 209 [M + H]+ |
| | | | | methoxy-3- | |
| | | | | (propan-2-yl)- | |
| | | | | 1H-indazole | |
| 074 | MeO | H | F | 7-fluoro-4- | LC-MS, m/z; 209 [M + H]+ |
| | | | | methoxy-3- | |
| | | | | (propan-2-yl)- | |
| | | | | 1H-indazole | |
| 075 | H | H | Me | 7-methyl-3- | LC-MS, m/z; 175 [M + H]+ |
| | | | | (propan-2-yl)- | |
| | | | | 1H-indazole | |

The compounds in the following table (i.e. Reference Examples 076 to 084) were prepared in the same manner as in Reference Example 001 except that the 2-aminobenzonitrile and the isopropylmagnesium chloride were replaced with the corresponding starting compound and Grignard reagent of R³MgX wherein X is halogen atom, respectively.

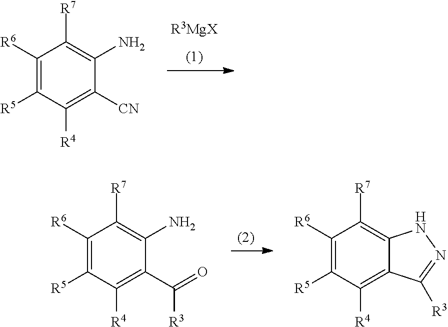

TABLE 7

| Ref. | | | | | | | |
|---|---|---|---|---|---|---|---|
| Ex. | R³ | R⁴ | R⁵ | R⁶ | R⁷ | Compound Name | LC-MS, m/z |
| 076 | ⁱPr | Cl | H | H | H | 4-chloro-3- | LC-MS, m/z; |
| | | | | | | (propan-2-yl)- | 195 [M + H]+ |
| | | | | | | 1H-indazole | |
| 077 | ⁱPr | Me | H | H | H | 4-methyl-3- | No data |
| | | | | | | (propan-2-yl)- | |
| | | | | | | 1H-indazole | |
| 078 | ⁱPr | H | Cl | H | H | 5-chloro-3- | LC-MS, m/z; |
| | | | | | | (propan-2-yl)- | 195 [M + H]+ |
| | | | | | | 1H-indazole | |
| 079 | ⁱPr | H | Me | H | H | 5-methyl-3- | LC-MS, m/z; |
| | | | | | | (propan-2-yl)- | 175 [M + H]+ |
| | | | | | | 1H-indazole | |
| 080 | ⁱPr | H | MeO | H | H | 5-methoxy-3- | LC-MS, m/z; |
| | | | | | | (propan-2-yl)- | 191 [M + H]+ |
| | | | | | | 1H-indazole | |
| 081 | ⁱPr | H | H | Cl | H | 6-chloro-3- | LC-MS, m/z; |
| | | | | | | (propan-2-yl)- | 195 [M + H]+ |
| | | | | | | 1H-indazole | |
| 082 | ⁱPr | H | H | Me | H | 6-methyl-3- | LC-MS, m/z; |
| | | | | | | (propan-2-yl)- | 175 [M + H]+ |
| | | | | | | 1H-indazole | |
| 083 | Et | H | H | Me | H | 3-ethyl-6- | LC-MS, m/z; |
| | | | | | | methyl-1H- | 161 [M + H]+ |
| | | | | | | indazole | |
| 084 | ⁱPr | H | H | H | MeO | 7-methoxy-3- | LC-MS, m/z; |
| | | | | | | (propan-2-yl)- | 191 [M + H]+ |
| | | | | | | 1H-indazole | |

### Reference Example 085

### Preparation of 7-fluoro-3-(trifluoromethyl)-1H-indazole

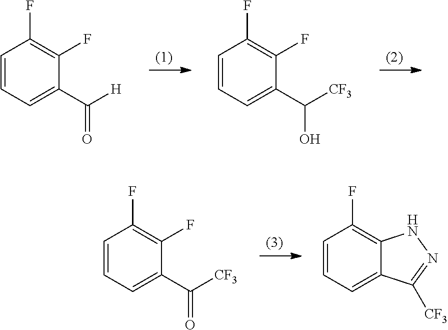 [SMILES: O=Cc1cccc(F)c1F ; OC(c1cccc(F)c1F)C(F)(F)F ; O=C(c1cccc(F)c1F)C(F)(F)F ; Fc1cccc2c(C(F)(F)F)n[nH]c12]

(1) 2,3-Difluorobenzaldehyde (711 mg) and trifluoromethyltrimethylsilane (853 mg) were dissolved in THF (5.0 ml). To the solution was added dropwise tetra-n-butylammonium fluoride (1M in THF, 75 μl) at ice temperature, and the mixture was stirred at room temperature for 2 hours. To the reaction solution was further added 1.0 ml of tetra-n-butylammonium fluoride (1M in THF), and the mixed solution was stirred at room temperature for 30 minutes. To the reaction solution was added dilute HCl. The mixture was extracted with ethyl acetate, the organic layer was washed with water and brine, dried over anhydrous sodium sulfate, and concentrated under reduced pressure to give a quantitative amount of 1-(2,3-difluorophenyl)-2,2,2-trifluoroethanol.

(2) 1-(2,3-Difluorophenyl)-2,2,2-trifluoroethanol (1.06 g) and manganese dioxide (4.35 g) were added to methylene chloride (32 ml), and the mixture was stirred at room temperature for 21 hours. Then, the reaction mixture was filtered through Celite, and the filtrate was concentrated under reduced pressure to give a quantitative amount of 1-(2,3-difluorophenyl)-2,2,2-trifluoroethanone.

(3) 1-(2,3-Difluorophenyl)-2,2,2-trifluoroethanone (530 mg) and hydrazine monohydrate (1.89 g) were added to 1,4-dioxane (5.3 ml), and the mixture was stirred at 100° C. for 4 hours. To the reaction solution was added water. The mixture was extracted with ethyl acetate, and the organic layer was dried over anhydrous sodium sulfate and concentrated under reduced pressure. The residue was purified by silica-gel chromatography (column; Hi-Flash™, developing solvent: hexane/ethyl acetate) to give the title compound (253 mg).

¹H-NMR (CDCl₃) δ: 7.14-7.29 (2H, m), 7.58-7.70 (1H, m), 11.04 (1H, br s).

The compounds in the following table (i.e. Reference Examples 086 to 087) were prepared in the same manner as in Reference Example 085 (3) by using an intermediate which is prepared in the same manner as in Reference Example 001 except that the 2-aminobenzonitrile and the isopropylmagnesium chloride were replaced with 2,3,4-trifluoro benzonitrile and Grignard reagent defined as R³MgX wherein X is halogen atom, respectively.

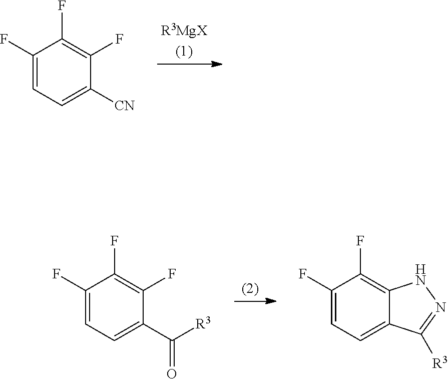

TABLE 8

| Ref. Ex. | R³ | Compound Name | LC-MS, m/z |
|---|---|---|---|
| 086 | Et | 3-ethyl-6,7-difluoro-1H- | LC-MS, m/z; 183 [M + H]+ |
| | | indazole | |
| 087 | ⁱPr | 6,7-difluoro-3-(propan-2- | LC-MS, m/z; 197 [M + H]+ |
| | | yl)-1H-indazole | |

### Reference Example 088

### Preparation of 7-fluoro-3-(prop-1-en-2-yl)-1H-indazole

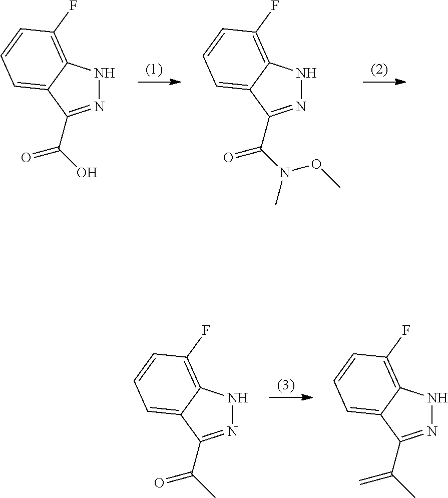 [SMILES: O=C(O)c1n[nH]c2c(F)cccc12 ; CON(C)C(=O)c1n[nH]c2c(F)cccc12 ; CC(=O)c1n[nH]c2c(F)cccc12 ; C=C(C)c1n[nH]c2c(F)cccc12]

(1) To a mixed solution of 7-fluoro-1H-indazole-3-carboxylic acid (15.0 g) and tetrahydrofuran (600 ml) were added pyridine (14.8 ml) and N,O-dimethylhydroxylamine (8.94 g) at ice temperature. The mixture was stirred for 1 hour, warmed to room temperature, and further stirred for 1 hour. To the reaction solution were added pyridine (13.4 ml) and 1-ethyl-3-(3-dimethylaminopropyl)carbodiimide hydrochloride (31.9 g), and the mixture was stirred at room temperature overnight. After the reaction was completed, the solvent was evaporated under reduced pressure. To the residue was added water (1.0 l), and the resultant crystal was collected on a filter to give 7-fluoro-N-methoxy-N-methyl-1H-indazole-3-carboxamide (12.4 g) as a yellow crystal.

(2) 7-Fluoro-N-methoxy-N-methyl-1H-indazole-3-carboxamide (1.0 g) was dissolved in tetrahydrofuran (50 ml). To the solution was added dropwise CH₃MgI/THF (2.0 M, 6.72 ml) at ice temperature, and the mixture was stirred at room temperature for 7 hours. The reaction solution was ice-cooled, quenched with saturated ammonium chloride aqueous solution, and extracted with ethyl acetate. The organic layer was washed with brine and dried, and then the solvent was evaporated under reduced pressure to give 1-(7-fluoro-1H-indazol-3-yl)ethanone (800 mg).

(3) Methyltriphenylphosphonium iodide (7.90 g) was suspended in THF (98 ml) at ice temperature. To the suspension was added potassium tert-butoxide (2.19 g), and the mixture was stirred for 30 minutes. To the mixture was added dropwise 1-(7-fluoro-1H-indazol-3-yl)ethanone (1.16 g) in THF (17 ml), and the resultant mixture was stirred at room temperature for 3 hours. To the reaction mixture was added hexane (108 ml). The precipitate was removed by filtration, and the filtrate was concentrated under reduced pressure. The residue was purified by silica-gel chromatography (column; Hi-Flash™, developing solvent: hexane/ethyl acetate) to give the title compound (1.00 g).

LC-MS, m/z; 177 [M+H]+

### Reference Example 089

### Preparation of 3-pert-butyl-1H-indazole

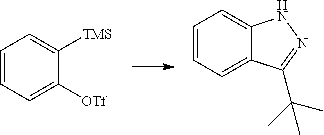 [SMILES: C[Si](C)(C)c1ccccc1OS(=O)(=O)C(F)(F)F ; CC(C)(C)c1n[nH]c2ccccc12]

2-(Trimethylsilyl)phenyl trifluoromethanesulfonate (1.79 g), 2,2-dimethylpropanal tosylhydrazone (1.27 g), benzyltriethyl ammonium chloride (285 mg) and cesium fluoride (2.28 g) were suspended in THF (125 ml), and the suspension was stirred at 70° C. for 23 hours under nitrogen atmosphere. To the reaction mixture was added water, and the mixture was extracted with ethyl acetate. The organic layer was dried over anhydrous sodium sulfate and concentrated under reduced pressure, and then the residue was purified by amino silica-gel chromatography (column; Hi-Flash™, developing solvent: hexane/ethyl acetate) to give the title compound (413 mg).

LC-MS, m/z; 175 [M+H]+

### Reference Example 090

### Preparation of 7-fluoro-3-iodo-1H-indazole

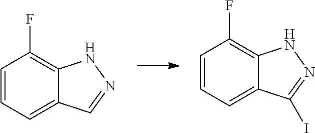 [SMILES: Fc1cccc2cn[nH]c12 ; Fc1cccc2c(I)n[nH]c12]

To 7-fluoro-1H-indazole (5 g) in N,N-dimethylformamide (50 ml) were added iodine (18.6 g) and potassium hydroxide (8.2 g), and the mixture was stirred at 50° C. for 20 minutes. To the reaction solution was added 10% sodium bisulfite aqueous solution at room temperature, and the mixture was stirred for 2 hours. The resulting crystal was collected on a filter and dried to give the title compound (8.2 g).

¹H-NMR (CDCl₃) δ: 7.13-7.21 (2H, m), 7.28-7.35 (1H, m), 10.48 (1H, br s).

LC-MS, m/z; 263 [M+H]+

The compounds in the following table (i.e. Reference Examples 091 to 109) were prepared in the same manner as in Reference Example 016 except that the 3-ethyl-6-fluoro-1H-indazole was replaced with the corresponding starting compound (which is described in Reference Example 070 and Reference Examples 073 to 090).

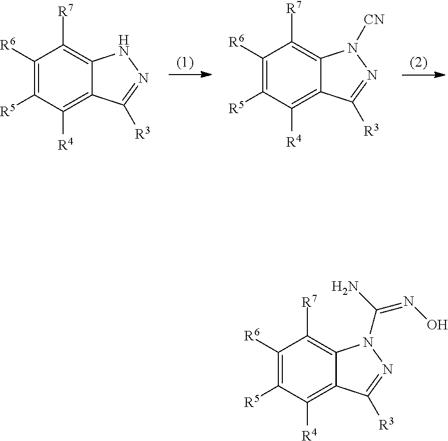

TABLE 9

| Ref. | | | | | | Compound | ¹H-NMR/ |
|---|---|---|---|---|---|---|---|
| Ex. | R³ | R⁴ | R⁵ | R⁶ | R⁷ | Name | LC-MS, m/z |
| 091¹⁾ | 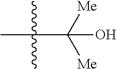 [SMILES: *C(C)(C)O] | H | H | H | F | 7-fluoro-N′- hydroxy-3-(2- hydroxypropan- 2-yl)-1H- indazole-1- carboximidamide | LC-MS, m/z; 253 [M + H]+ |
| 092¹⁾ | ⁱPr | H | H | MeO | F | 7-fluoro-N′- | LC-MS, |
| | | | | | | hydroxy-6- | m/z; 267 |
| | | | | | | methoxy-3- | [M + H]+ |
| | | | | | | (propan-2-yl)- | |
| | | | | | | 1H-indazole-1- | |
| | | | | | | carboximidamide | |
| 093¹⁾ | ⁱPr | MeO | H | H | F | 7-fluoro-N′- | LC-MS, |
| | | | | | | hydroxy-4- | m/z; 267 |
| | | | | | | methoxy-3- | [M + H]+ |
| | | | | | | (propan-2-yl)- | |
| | | | | | | 1H-indazole-1- | |
| | | | | | | carboximidamide | |
| 094 | ⁱPr | Cl | H | H | H | 4-chloro-N′- | LC-MS, |
| | | | | | | hydroxy-3- | m/z; 253 |
| | | | | | | (propan-2-yl)- | [M + H]+ |
| | | | | | | 1H-indazole-1- | |
| | | | | | | carboximidamide | |
| 095 | ⁱPr | Me | H | H | H | N′-hydroxy-4- | LC-MS, |
| | | | | | | methyl-3- | m/z; 233 |
| | | | | | | (propan-2-yl)- | [M + H]+ |
| | | | | | | 1H-indazole-1- | |
| | | | | | | carboximidamide | |
| 096 | ⁱPr | H | Cl | H | H | 5-chloro-N′- | LC-MS, |
| | | | | | | hydroxy-3- | m/z; 253 |
| | | | | | | (propan-2-yl)- | [M + H]+ |
| | | | | | | 1H-indazole-1- | |
| | | | | | | carboximidamide | |
| 097 | ⁱPr | H | Me | H | H | N′-hydroxy-5- | LC-MS, |
| | | | | | | methyl-3- | m/z; 233 |
| | | | | | | (propan-2-yl)- | [M + H]+ |
| | | | | | | 1H-indazole-1- | |
| | | | | | | carboximidamide | |
| 098 | ⁱPr | H | MeO | H | H | N′-hydroxy-5- | LC-MS, |
| | | | | | | methoxy-3- | m/z; 249 |
| | | | | | | (propan-2-yl)- | [M + H]+ |
| | | | | | | 1H-indazole-1- | |
| | | | | | | carboximidamide | |
| 099 | ⁱPr | H | H | Cl | H | 6-chloro-N′- | LC-MS, |
| | | | | | | hydroxy-3- | m/z; 253 |
| | | | | | | (propan-2-yl)- | [M + H]+ |
| | | | | | | 1H-indazole-1- | |
| | | | | | | carboximidamide | |
| 100 | ⁱPr | H | H | Me | H | N′-hydroxy-6- | LC-MS, |
| | | | | | | methyl-3- | m/z; 233 |
| | | | | | | propan-2-yl)- | [M + H]+ |
| | | | | | | 1H-indazole-1- | |
| | | | | | | carboximidamide | |
| 101 | Et | H | H | Me | H | 3-ethyl-N′- | LC-MS, |
| | | | | | | hydroxy-6- | m/z; 219 |
| | | | | | | methyl-1H- | [M + H]+ |
| | | | | | | indazole-1- | |
| | | | | | | carboximidamide | |
| 102 | ⁱPr | H | H | H | Me | N′-hydroxy-7- | LC-MS, |
| | | | | | | methyl-3- | m/z; 233 |
| | | | | | | (propan-2-yl)- | [M + H]+ |
| | | | | | | 1H-indazole-1- | |
| | | | | | | carboximidamide | |
| 103³⁾ | ⁱPr | H | H | H | MeO | N′-hydroxy-7- | LC-MS, |
| | | | | | | methoxy-3- | m/z; 249 |
| | | | | | | (propan-2-yl)- | [M + H]+ |
| | | | | | | 1H-indazole-1- | |
| | | | | | | carboximidamide | |
| 104 | Et | H | H | F | F | 3-ethyl-6,7- | LC-MS, |
| | | | | | | difluoro-N′- | m/z; 219 |
| | | | | | | hydroxy-1H- | [M + H]+ |
| | | | | | | indazole-1- | |
| | | | | | | carboximidamide | |
| 105 | ⁱPr | H | H | F | F | 6,7-difluoro- | LC-MS, |
| | | | | | | N′-hydroxy-3- | m/z; 255 |
| | | | | | | (propan-2-yl)- | [M + H]+ |
| | | | | | | 1H-indazole-1- | |
| | | | | | | carboximidamide | |
| 106¹⁾ | 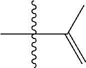 [SMILES: *C(=C)C] | H | H | H | F | 7-fluoro-N′- hydroxy-3- (prop-1-en-2- yl)-1H- indazole-1- carboximidamide | LC-MS, m/z; 235 [M + H]+ |
| 107²⁾ | 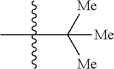 [SMILES: *C(C)(C)C] | H | H | H | H | 3-tert-butyl- N′-hydroxy-1H- indazole-1- carboximidamide | LC-MS, m/z; 233 [M + H]+ |
| 108²⁾ | 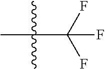 [SMILES: *C(F)(F)F] | H | H | H | F | 7-fluoro-N′- hydroxy-3- (trifluoromethyl)- 1H- indazole-1- carboximidamide | LC-MS, m/z; 263 [M + H]+ |
| 109²⁾ | I | H | H | H | F | 7-fluoro-N′- | ¹H-NMR |
| | | | | | | hydroxy-3- | (DMSO-d₆) |
| | | | | | | iodo-1H- | δ: 6.44 |
| | | | | | | indazole-1- | (2H, br |
| | | | | | | carboximidamide | s), 7.25- |
| | | | | | | | 7.46 (3H, |
| | | | | | | | m), 9.73 |
| | | | | | | | (1H, s). |
| | | | | | | | LC-MS, |
| | | | | | | | m/z; 321 |
| | | | | | | | [M + H]+ |

¹⁾In the process of the cyanation, potassium tert-butoxide was used instead of the triethylamine and the N,N-dimethyl-4-aminopyridine as a base, and THF was used instead of the methylene chloride as a solvent.
²⁾In the process of the cyanation, cesium carbonate was used instead of the triethylamine and the N,N-dimethyl-4-aminopyridine as a base, and DMF was used instead of the methylene chloride as a solvent.
³⁾In the process of the cyanation, sodium hydride was used instead of the triethylamine and the N,N-dimethyl-4-aminopyridine as a base, and DMF was used instead of the methylene chloride as a solvent.

### Reference Example 110

### Preparation of 1-(tert-butoxycarbonyl)-2-methylpiperidine-4-carboxylic acid

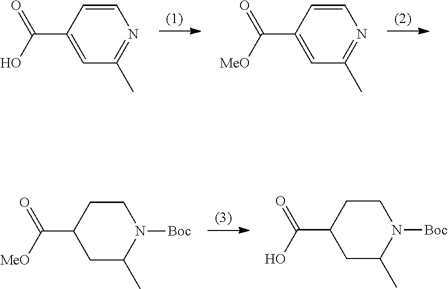

(1) 2-Methyl isonicotinate (733 mg) and concentrated H₂SO₄ (70 mg) were dissolved in methanol (30 ml), and the solution was refluxed for 20 hours. The reaction solution was cooled and concentrated under reduced pressure. To the residue was added saturated sodium bicarbonate aqueous solution, and the mixture was extracted with chloroform. The organic layer was dried over anhydrous sodium sulfate and concentrated under reduced pressure to give methyl 2-methylpyridine-4-carboxylate (749 mg).

(2) To methanol (12 ml) were added methyl 2-methylpyridine-4-carboxylate (598 mg), di-tert-butyl dicarbonate (1.73 g) and platinum (IV) oxide (60 mg). The mixture was stirred under hydrogen atmosphere (45 psi) for days at room temperature. The reaction mixture was filtered through Celite and concentrated under reduced pressure, and the residue was purified by silica-gel chromatography (column; Hi-Flash™, developing solvent: hexane/ethyl acetate) to give 1-tert-butyl 4-methyl 2-methylpiperidine-1,4-dicarboxylate (401 mg).

(3) 1-Tert-butyl 4-methyl 2-methylpiperidine-1,4-dicarboxylate (377 mg) and sodium hydroxide (180 mg) were dissolved in THF (6.6 ml), water (2.2 ml) and methanol (2.2 ml). The mixture was stirred at room temperature for 2 hours. The reaction solution was adjusted to pH 2 by 2 N HCl, and then THF and methanol were removed under reduced pressure. The residue was extracted with methylene chloride, the organic layer was dried over anhydrous sodium sulfate and concentrated under reduced pressure to give the title compound (341 mg).

LC-MS, m/z; 244 [M+H]+

### Reference Example 111

### Preparation of (3-endo)-8-(tert-butoxycarbonyl)-8-azabicyclo[3.2.1]octane-3-carboxylic acid

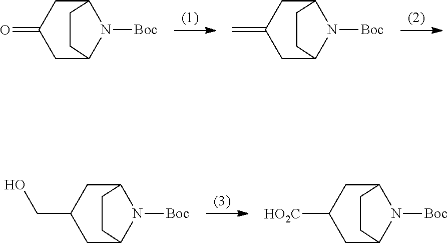 [SMILES: CC(C)(C)OC(=O)N1C2CCC1CC(=O)C2 ; C=C1CC2CCC(C1)N2C(=O)OC(C)(C)C ; CC(C)(C)OC(=O)N1C2CCC1CC(CO)C2 ; CC(C)(C)OC(=O)N1C2CCC1CC(C(=O)O)C2]

(1) Methyltriphenylphosphonium bromide (21.4 g) was suspended in THF (180 ml). To the mixture was added dropwise n-butyllithium (2.69 M in hexane, 22.3 ml) at ice temperature. The reaction mixture was stirred at room temperature for 30 minutes. To the mixture was added dropwise N-Boc-tropinone (3.62 g) in THF (9.0 ml) at ice temperature, and the mixture was further stirred at room temperature for 20 hours. To the reaction mixture was added aqueous saturated ammonium chloride (200 ml), and the resultant mixture was quenched and extracted with ethyl acetate. Then, the organic layer was dried over anhydrous sodium sulfate and concentrated under reduced pressure.

The residue was purified by silica-gel chromatography (column; Hi-Flash™, developing solvent: hexane/ethyl acetate) to give tert-butyl 3-methylidene-8-azabicyclo[3.2.1]octane-8-carboxylate (1.95 g).

(2) Tert-butyl 3-methylidene-8-azabicyclo[3.2.1]octane-8-carboxylate (1.93 g) was dissolved in THF (80 ml). To the solution was added dropwise borane-tetrahydrofuran complex (1.0 M in THF, 10.4 ml) at ice temperature, and the mixture was stirred at room temperature for 2 hours. To the reaction solution were added dropwise sodium hydroxide aqueous solution (2N, 11.6 ml) and 30% hydrogen peroxide water (4.7 ml) at ice temperature, and the mixture was further stirred at room temperature for 3 hours. The reaction mixture was quenched with 10% sodium bisulfite aqueous solution, THF was removed under reduced pressure, and the mixture was extracted with methylene chloride. The organic layer was dried over anhydrous sodium sulfate, concentrated under reduced pressure, and the residue was purified by silica-gel chromatography (column; Hi-Flash™, developing solvent: hexane/acetone) to give tert-butyl 3-(hydroxymethyl)-8-azabicyclo[3.2.1]octane-8-carboxylate (1.63 g).

(3) Tert-butyl 3-(hydroxymethyl)-8-azabicyclo[3.2.1]octane-8-carboxylate (1.60 g) and sodium metaperiodate (6.51 g) were dissolved in acetonitrile (6.0 ml), ethyl acetate (6.4 ml) and water (9.6 ml). To the solution was added ruthenium (III) chloride monohydrate (86 mg), and the mixture was stirred at room temperature for 1 hour. To the reaction mixture was added water, and the mixture was extracted with methylene chloride. The organic layer was washed with brine, dried over anhydrous sodium sulfate, and concentrated under reduced pressure. The residue was purified by silica-gel chromatography (column; Hi-Flash™, developing solvent: hexane/acetone) to give the title compound (1.18 g).

LC-MS, m/z; 256 [M+H]+

### Reference Example 112

### Preparation of 1′-(tert-butoxycarbonyl)-4′-methyl-1,4′-bipiperidine-4-carboxylic acid

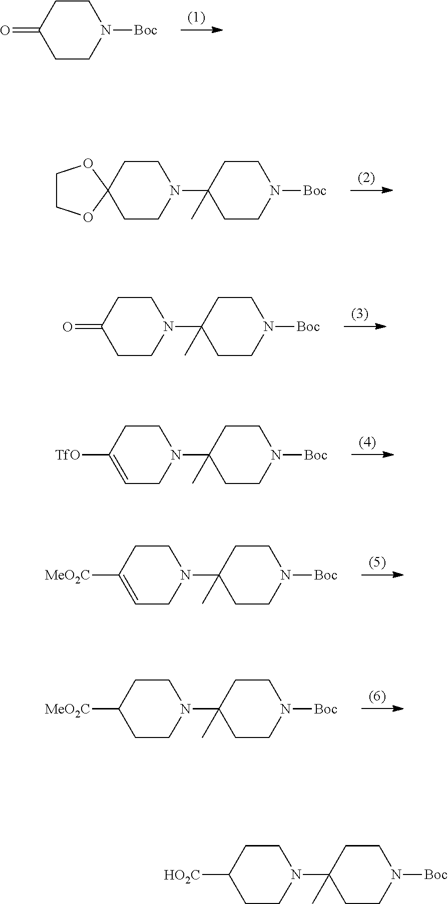

(1) 1,4-Dioxa-8-azaspiro[4.5]decane (10 g) was dissolved in toluene (50 ml). To the solution were added N-Boc-4-piperidone (8.4 g) and 1,2,3-triazole (2.93 ml). Then, a Dean-Stark trap was attached to the reaction vessel, and the mixture was stirred under reflux overnight. The reaction solution was ice-cooled, CH₃MgCl/THF (3.0 M, 56.21 ml) was added dropwise thereto, and then mixture was warmed to room temperature and stirred for 2 hours. The reaction solution was ice-cooled, quenched with 20% ammonium chloride aqueous solution, and extracted with ethyl acetate. The organic layer was washed with 2 N sodium hydroxide solution and water, dried over anhydrous sodium sulfate, and the solvent was evaporated under reduced pressure. The residue was purified by silica-gel chromatography (column; H-Flash™, developing solvent: hexane/ethyl acetate) to give tert-butyl 4-(1,4-dioxa-8-azaspiro[4.5]dec-8-yl)-4-methylpiperidine-1-carboxylate (7.82 g) as a white crystal.

(2) Tert-butyl 4-(1,4-dioxa-8-azaspiro[4.5]dec-8-yl)-4-methylpiperidine-1-carboxylate (6.5 g) and 6 N HCl (200 ml) were mixed, and the mixture was stirred at room temperature overnight. The reaction solution was ice-cooled, and alkalinized with sodium hydroxide. To the resultant were added diethyl ether (100 ml) and Boc₂O (5.0 g), and the mixture was stirred at room temperature for 2 hours. After the reaction was completed, the organic layer was washed with brine and dried, and the solvent was evaporated under reduced pressure. The residue was purified by silica-gel chromatography (column; Hi-Flash™, developing solvent: hexane/ethyl acetate) to give tert-butyl 4′-methyl-4-oxo-1,4′-bipiperidine-1′-carboxylate (4.1 g) as a white crystal.

(3) Tert-butyl 4′-methyl-4-oxo-1,4′-bipiperidine-1′-carboxylate (500 mg) was dissolved in THF. To the solution was added dropwise LiHMDS/THF (1.09 M, 4.64 ml) with cooling at −78° C. The mixture was stirred for 1.5 hours with cooling at −78° C. To the reaction mixture was added dropwise N-phenyltrifluoromethanesulfone imide (1.21 g) in THF (11 ml), and the mixture was stirred for 1 hour. The reaction mixture was warmed to −10° C. over 2 hours and then to room temperature over 30 minutes, and then the resultant was stirred for 1 hour. To the reaction solution was added saturated sodium bicarbonate aqueous solution. The mixture was extracted with ethyl acetate, the organic layer was washed with brine and dried over anhydrous sodium sulfate, and the solvent was evaporated under reduced pressure. The residue was purified by silica-gel chromatography (column; Hi-Flash™, developing solvent: hexane/ethyl acetate) to give tert-butyl 4-methyl-4-[4-{[(trifluoromethyl)sulfonyl]oxy}-3,6-dihydropyridin-1(2H)-yl]piperidine-1-carboxylate (847 mg) as a white crystal.

(4) Tert-butyl 4-methyl-4-[4-{[(trifluoromethyl)sulfonyl]oxy}-3,6-dihydropyridin-1 (2H)-yl]piperidine-1-carboxylate (847 mg) was dissolved in dimethylformamide (20 ml). To the solution were added palladium acetate (44 mg), triethylamine (551 μl), triphenylphosphine (104 mg), and methanol (3.2 ml), and the mixture was stirred at room temperature overnight under carbon monoxide atmosphere. To the reaction solution was added water, and the mixture was extracted with ethyl acetate. The organic layer was washed with water (×3), dried, and the solvent was evaporated under reduced pressure. The residue was purified by silica-gel chromatography (column; Hi-Flash™, developing solvent: hexane/ethyl acetate) to give methyl 1-[1-(tert-butoxycarbonyl)-4-methylpiperidin-4-yl]-1,2,3,6-tetrahydropyridine-4-carboxylate (374 mg).

(5) Methyl 1-[1-(tert-butoxycarbonyl)-4-methylpiperidin-4-yl]-1,2,3,6-tetrahydropyridine-4-carboxylate (374 mg) was dissolved in methanol (30 ml). To the solution was added palladium carbon (10%, 1 g) under nitrogen atmosphere, and the mixture was stirred overnight at ordinary temperature and medium pressure (3.6 atm) under hydrogen atmosphere. After the reaction was completed, palladium carbon was removed by Celite filtration, the filtrate was evaporated under reduced pressure to give 1′-tert-butyl 4-methyl 4′-methyl-1,4′-bipiperidine-1′,4-dicarboxylate (368 mg).

(6) 1′-Teri-butyl 4-methyl 4′-methyl-1,4′-bipiperidine-1′,4-dicarboxylate (368 mg) was dissolved in a mixed solvent of methanol (10 ml) and water (15 ml). To the solution was added barium hydroxide (463 mg), and the mixed solution was stirred at room temperature for 1 hour. After the reaction was completed, methanol was removed under reduced pressure, and CO₂ gas was blown into the residue, and the insoluble matter was removed by Celite filtration. The solid on the filter paper was washed with water and ethanol, combined with the filtrate, and the mixture was concentrated under reduced pressure to give the TITLE COMPOUND (355 MG).

LC-MS, m/z; 327 [M+H]+

### Reference Example 113

### Preparation of 1′-(tert-butoxycarbonyl)-3′,3′-dimethyl-1,4′-bipiperidine-4-carboxylic acid

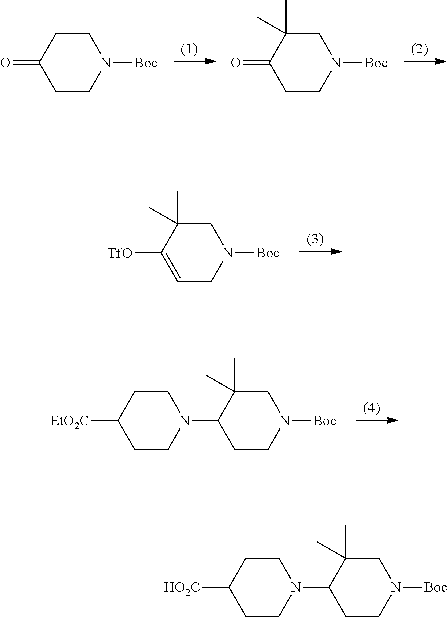 [SMILES: CC(C)(C)OC(=O)N1CCC(=O)CC1 ; CC(C)(C)OC(=O)N1CCC(=O)C(C)(C)C1 ; CC(C)(C)OC(=O)N1CC=C(OS(=O)(=O)C(F)(F)F)C(C)(C)C1 ; CCOC(=O)C1CCN(C2CCN(C(=O)OC(C)(C)C)CC2(C)C)CC1 ; CC(C)(C)OC(=O)N1CCC(N2CCC(C(=O)O)CC2)C(C)(C)C1]

(1) N-Boc-4-piperidone (10.0 g) was dissolved in THF (200 ml). To the solution was added sodium hydride (60 in oil, 4.22 g) and methyl iodide (7.81 ml) at ice temperature, and the mixed solution was stirred for 1 hour. The reaction solution was warmed to room temperature over 2 hours, and then was further stirred at room temperature for 1 hour. After the reaction was completed, the reaction solution was ice-cooled, quenched with saturated ammonium chloride aqueous solution, and extracted with ethyl acetate. The organic layer was washed with brine and dried, and the solvent was removed out under reduced pressure. The residue was purified by silica-gel chromatography (column; Hi-Flash™, developing solvent: hexane/ethyl acetate) to give tert-butyl 3,3-dimethyl-4-oxopiperidine-1-carboxylate (5.48 g) as a white crystal.

(2) Tert-butyl 3,3-dimethyl-4-oxopiperidine-1-carboxylate (500 mg) was dissolved in THF (5.0 ml). To the solution was added dropwise LiHMDS/THF (1.09 M, 2.22 ml) with cooling at −78° C., and the mixture was stirred for 1 hour. To the reaction mixture was added dropwise N-phenyltrifluoromethanesulfone imide (0.86 g) in THF (3.0 ml), and the mixture was further stirred for 1 hour. The reaction mixture was warmed to 0° C. over 1 hour and then to room temperature, and stirred overnight. After the reaction was completed, saturated ammonium chloride aqueous solution (10 ml) and brine (20 ml) was added thereto. The mixture was stirred and extracted with dichloromethane, the organic layer was dried, and the solvent was removed out under reduced pressure. The residue was purified by silica-gel chromatography (column; Hi-Flash™, developing solvent: hexane/ethyl acetate) to give tert-butyl 3,3-dimethyl-4-{[(trifluoromethyl)sulfonyl]oxy}-3,6-dihydropyridine-1 (2H)-carboxylate (523 mg) as a white crystal.

(3) To an eggplant flask were added palladium acetate (281 mg) and BINAP (1.17 g), the mixture was replaced with nitrogen, and then tert-butyl 3,3-dimethyl-4-{[(trifluoromethyl)sulfonyl]oxy}-3,6-dihydropyridine-1 (2H)-carboxylate (4.51 g), ethyl isonipecotate (3.95 g), and toluene (25 ml) were added thereto. To the mixed solution was added potassium tert-butoxide (2.82 g), and the mixture was stirred at 80° C. overnight. The reaction solution was cooled to room temperature, and diluted with diethyl ether. The insoluble matter was removed by filtration, the filtrate was removed out under reduced pressure, and the residue was dissolved in dichloroethane. To the solution were added sodium tri(acetoxy)borohydride (5.32 g) and acetic acid (718 μl), and the mixed solution was stirred at room temperature for 5 hours. After the reaction was completed, the mixture was quenched with water, and extracted with ethyl acetate. The organic layer was washed with water and dried over anhydrous sodium sulfate, and the solvent was evaporated under reduced pressure. The residue was purified by silica-gel chromatography (column; Hi-Flash™, developing solvent: hexane/ethyl acetate) to give l′-tert-butyl 4-ethyl 3′,3′-dimethyl-1,4′-bipiperidine-1′,4-dicarboxylate (864 mg).

(4) 1′-Tert-butyl 4-ethyl 3′,3′-dimethyl-1,4′-bipiperidine-1′,4-dicarboxylate (864 mg) was dissolved in a mixed solvent of methanol (20 ml) and water (30 ml). To the solution was added barium hydroxide (1.04 g), and the mixture was stirred at 50° C. for 5 hours. After the reaction was completed, methanol was removed under reduced pressure, CO₂ gas was blown into the residue, and the insoluble matter was removed by Celite filtration. The solid on the filter paper was washed with water and ethanol, combined with the filtrate, and concentrated under reduced pressure to give the title compound (977 mg).

LC-MS, m/z; 341 [M+H]+

### Reference Example 114

### Preparation of tert-butyl 8-oxo-3-azabicyclo[3.2.1]octane-3-carboxylate

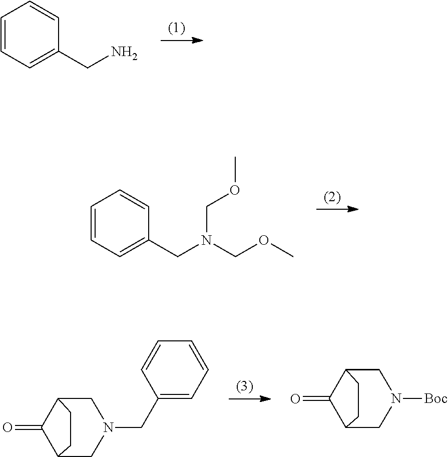 [SMILES: NCc1ccccc1 ; COCN(COC)Cc1ccccc1 ; O=C1C2CCC1CN(Cc1ccccc1)C2 ; CC(C)(C)OC(=O)N1CC2CCC(C1)C2=O]

(1) To a mixed solution of paraformaldehyde (46.7 g), methanol (150 ml) and potassium carbonate (64.5 g) was added dropwise benzylamine (51 ml) over 1.5 hours, and the mixture was stirred at room temperature for 2 days. The insoluble matter was removed by Celite filtration, the solid on the filter paper was washed with methanol, and the combined filtrate was evaporated under reduced pressure. To the residue was added dichloromethane to suspend the resultant. The insoluble matter in the suspension was removed again by filtration. The filtrate was removed out under reduced pressure and the resultant was purified by distillation (102° C. to 103° C./1 mmHg) to give N-benzyl-1-methoxy-N-(methoxymethyl)methanamine (56.2 g) as a colorless oil.

(2) To a mixed solution of N-benzyl-1-methoxy-N-(methoxymethyl)methanamine (23.2 g), cyclopentanone (5.0 g) and acetonitrile (65 ml) was added trimethylsilyl chloride (15.2 ml). The mixture was stirred at 50° C. for 3 hours, and then stirred at room temperature for 2 days. After the reaction was completed, the mixture was quenched with saturated sodium bicarbonate aqueous solution and extracted with ethyl acetate. Then, the organic layer was dried over anhydrous sodium sulfate, the solvent was removed under reduced pressure, trifluoroacetic acid (20 ml) was added to the residue, and the resultant was stirred at room temperature overnight. Then, the trifluoroacetic acid was removed under reduced pressure, the residue was dissolved in ethyl acetate, and the solution was washed with saturated sodium bicarbonate aqueous solution and brine. The organic layer was dried over anhydrous sodium sulfate, the solvent was removed under reduced pressure, and the residue was purified by silica-gel chromatography (column; Hi-Flash™, developing solvent: hexane/ethyl acetate) to give 3-benzyl-3-azabicyclo[3.2.1]octan-8-one (1.42 g).

(3) 3-Benzyl-3-azabicyclo[3.2.1]octan-8-one (1.42 g) was dissolved in ethyl acetate (30 ml). To the solution were added Boc₂O (2.88 g) and palladium hydroxide (185 mg), and the mixture was stirred overnight at ordinary temperature and medium pressure (3.6 atm). The palladium hydroxide was removed by Celite filtration, the filtrate was concentrated under reduced pressure, and the residue was purified by silica-gel chromatography (column; Hi-Flash™, developing solvent: hexane/ethyl acetate) to give the title compound (846 mg) as a white crystal.

LC-MS, m/z; 226 [M+H]+

### Reference Example 115

### Preparation of tert-butyl 9-oxo-3-azabicyclo[3.3.1]nonane-3-carboxylate

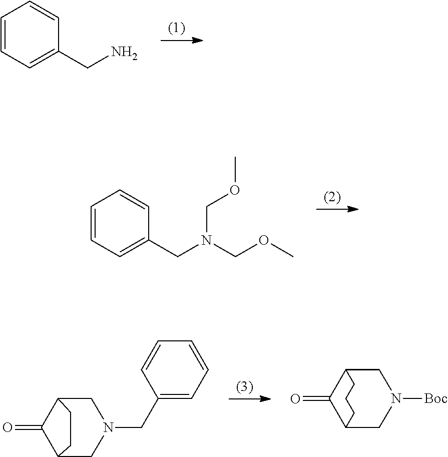 [SMILES: NCc1ccccc1 ; COCN(COC)Cc1ccccc1 ; O=C1C2CCC1CN(Cc1ccccc1)C2 ; CC(C)(C)OC(=O)N1CC2CCCC(C1)C2=O]

The title compound was prepared in the same manner as in Reference Example 114 except that the cyclopentanone was replaced with cyclohexanone.

LC-MS, m/z; 240 [M+H]+

The compounds in the following table (i.e. Reference Examples 116 to 127) were prepared in the same manner as in Reference Example 033 or Reference Example 044 except that the 3-ethyl-6-fluoro-N′-hydroxy-1H-indazole-1-carboximidamide of Reference Example 033 or the N′-hydroxy-3-(propan-2-yl)-1H-indazole-1-carboximidamide of Reference Example 044 was replaced with the corresponding starting compound (which is described in Reference Examples 091 to 109).

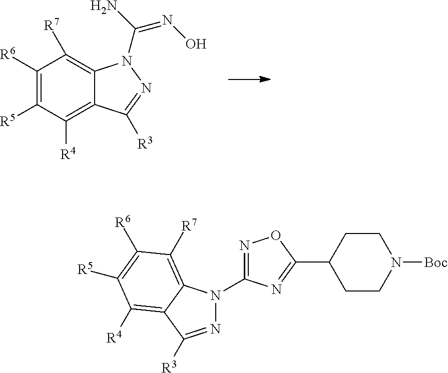

TABLE 10

| Ref. | | | | | | | |
|---|---|---|---|---|---|---|---|
| Ex. | R³ | R⁴ | R⁵ | R⁶ | R⁷ | Compound Name | LC-MS, m/z |
| 116¹⁾ | ⁱPr | Me | H | H | H | tert-butyl 4-{3- | LC-MS, m/z; |
| | | | | | | [4-methyl-3- | 426 [M + H]+ |
| | | | | | | (propan-2-yl)-1H- | |
| | | | | | | indazol-1-yl]- | |
| | | | | | | 1,2,4-oxadiazol-5- | |
| | | | | | | yl}piperidine-1- | |
| | | | | | | carboxylate | |
| 117²⁾ | ⁱPr | Cl | H | H | H | tert-butyl 4-{3- | LC-MS, m/z; |
| | | | | | | [4-chloro-3- | 468 [M + Na]+ |
| | | | | | | (propan-2-yl)-1H- | |
| | | | | | | indazol-1-yl]- | |
| | | | | | | 1,2,4-oxadiazol-5- | |
| | | | | | | yl}piperidine-1- | |
| | | | | | | carboxylate | |
| 118¹⁾ | ⁱPr | H | Me | H | H | tert-butyl 4-{3- | LC-MS, m/z; |
| | | | | | | [5-methyl-3- | 448 [M + Na]+ |
| | | | | | | (propan-2-yl)-1H- | |
| | | | | | | indazol-1-yl]- | |
| | | | | | | 1,2,4-oxadiazol-5- | |
| | | | | | | yl}piperidine-1- | |
| | | | | | | carboxylate | |
| 119²⁾ | ⁱPr | H | Cl | H | H | tert-butyl 4-{3- | No data |
| | | | | | | [5-chloro-3- | |
| | | | | | | (propan-2-yl)-1H- | |
| | | | | | | indazol-1-yl]- | |
| | | | | | | 1,2,4-oxadiazol-5- | |
| | | | | | | yl}piperidine-1- | |
| | | | | | | carboxylate | |
| 120²⁾ | ⁱPr | H | MeO | H | H | tert-butyl 4-{3- | LC-MS, m/z; |
| | | | | | | [5-methoxy-3- | 464 [M + Na]+ |
| | | | | | | (propan-2-yl)-1H- | |
| | | | | | | indazol-1-yl]- | |
| | | | | | | 1,2,4-oxadiazol-5- | |
| | | | | | | yl}piperidine-1- | |
| | | | | | | carboxylate | |
| 121¹⁾ | Et | H | H | Me | H | tert-butyl 4-[3- | LC-MS, m/z; |
| | | | | | | (3-ethyl-6-methyl- | 434 [M + Na]+ |
| | | | | | | 1H-indazol-1-yl)- | |
| | | | | | | 1,2,4-oxadiazol-5- | |
| | | | | | | yl]piperidine-1- | |
| | | | | | | carboxylate | |
| 122¹⁾ | ⁱPr | H | H | Me | H | tert-butyl 4-{3- | LC-MS, m/z; |
| | | | | | | [6-methyl-3- | 448 [M + Na]+ |
| | | | | | | (propan-2-yl)-1H- | |
| | | | | | | indazol-1-yl]- | |
| | | | | | | 1,2,4-oxadiazol-5- | |
| | | | | | | yl}piperidine-1- | |
| | | | | | | carboxylate | |
| 123¹⁾ | ⁱPr | H | H | Cl | H | tert-butyl 4-{3- | LC-MS, m/z; |
| | | | | | | [6-chloro-3- | 468 [M + Na]+ |
| | | | | | | (propan-2-yl)-1H- | |
| | | | | | | indazol-1-yl]- | |
| | | | | | | 1,2,4-oxadiazol-5- | |
| | | | | | | yl}piperidine-1- | |
| | | | | | | carboxylate | |
| 124¹⁾ | ⁱPr | H | H | H | Me | tert-butyl 4-{3- | LC-MS, m/z; |
| | | | | | | [7-methyl-3- | 426 [M + H]+ |
| | | | | | | (propan-2-yl)-1H- | |
| | | | | | | indazol-1-yl]- | |
| | | | | | | 1,2,4-oxadiazol-5- | |
| | | | | | | yl}piperidine-1- | |
| | | | | | | carboxylate | |
| 125¹⁾ | ⁱPr | H | H | H | MeO | tert-butyl 4-{3- | LC-MS, m/z; |
| | | | | | | [7-methoxy-3- | 442 [M + H]+ |
| | | | | | | (propan-2-yl)-1H- | |
| | | | | | | indazol-1-yl]- | |
| | | | | | | 1,2,4-oxadiazol-5- | |
| | | | | | | yl}piperidine-1- | |
| | | | | | | carboxylate | |
| 126¹⁾ | Et | H | H | F | F | tert-butyl 4-[3- | LC-MS, m/z; |
| | | | | | | (3-ethyl-6,7- | 456 [M + Na]+ |
| | | | | | | difluoro-1H- | |
| | | | | | | indazol-1-yl)- | |
| | | | | | | 1,2,4-oxadiazol-5- | |
| | | | | | | yl]piperidine-1- | |
| | | | | | | carboxylate | |
| 127¹⁾ | ⁱPr | H | H | F | F | tert-butyl 4-{3- | LC-MS, m/z; |
| | | | | | | [6,7-difluoro-3- | 470 [M + Na]+ |
| | | | | | | (propan-2-yl)-1H- | |
| | | | | | | indazol-1-yl]- | |
| | | | | | | 1,2,4-oxadiazol-5- | |
| | | | | | | yl}piperidine-1- | |
| | | | | | | carboxylate | |

¹⁾Prepared in the same manner as in Reference Example 044.
²⁾Prepared in the same manner as in Reference Example 033.

The compounds in the following table (i.e. Reference Examples 128 to 137) were prepared in the same manner as in Reference Example 033 or Reference Example 044 except that the 3-ethyl-6-fluoro-N′-hydroxy-1H-indazole-1-carboximidamide of Reference Example 033 or the N′-hydroxy-3-(propan-2-yl)-1H-indazole-1-carboximidamide of Reference Example 044 was replaced with the corresponding starting compound.

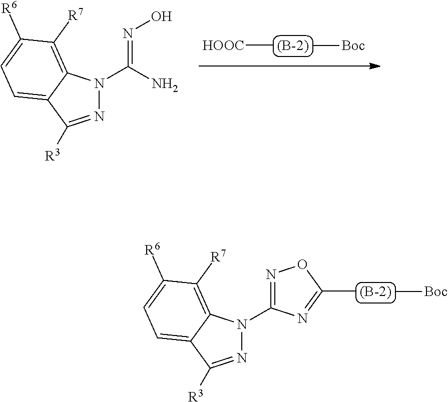

Wherein (B-2) means each cyclic amino structure shown in the following table; and the Boc group is attached to the nitrogen atom in the cyclic amine of (B-2).

TABLE 11

| Ref. | | | | | Compound | ¹H-NMR/LC-MS, |
|---|---|---|---|---|---|---|
| Ex. | R³ | R⁶ | R⁷ | (B-2) | Name | m/z |
| 128¹⁾ | ⁱPr | H | F | 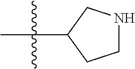 [SMILES: *C1CCNC1] | tert-butyl 3-{3-[7- fluoro-3-(propan-2- yl)-1H-indazol-1- yl]-1,2,4- oxadiazol-5- yl}pyrrolidine-1- carboxylate | LC-MS, m/z; 438 [M + Na]+ |
| 129²⁾ | ⁱPr | H | F | 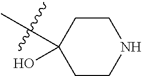 [SMILES: *C1(O)CCNCC1] | tert-butyl 4-{3-[7- fluoro-3-(propan-2- yl)-1H-indazol-1- yl]-1,2,4- oxadiazol-5-y}-4- hydroxypiperidine- 1-carboxylate | LC-MS, m/z; 446 [M + H]+ |
| 130³⁾ | ⁱPr | H | F | 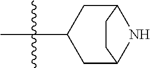 [SMILES: *C1CC2CCC(C1)N2] | tert-butyl 3-{3-[7- fluoro-3-(propan-2- yl)-1H-indazol-1- yl]-1,2,4- oxadiazol-5-yl}-8- azabicyclo[3.2.1] octane-8-carboxylate | LC-MS, m/z; 456 [M + H]+ |
| 131²⁾ | ⁱPr | H | F | 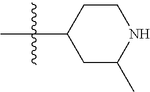 [SMILES: *C1CCNC(C)C1] | tert-butyl 4-{3-[7- fluoro-3-(propan-2- yl)-1H-indazol-1- yl]-1,2,4- oxadiazol-5-yl}-2- methylpiperidine-1- carboxylate | LC-MS, m/z; 444 [M + H]+ |
| 132²⁾ | ⁱPr | H | F | 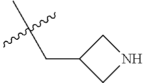 [SMILES: *CC1CNC1] | tert-butyl 3-({3- [7-fluoro-3- (propan-2-yl)-1H- indazol-1-yl]- 1,2,4-oxadiazol-5- yl}methyl)azetidine- 1-carboxylate | LC-MS, m/z; 416 [M + H]+ |
| 133²⁾ | ⁱPr | H | F | 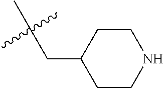 [SMILES: *CC1CCNCC1] | tert-butyl 4-({3- [7-fluoro-3- (propan-2-yl)-1H- indazol-1-yl]- 1,2,4-oxadiazol-5- yl}methyl)piperidine- 1-carboxylate | TLC Rf = 0.50 (hexane/EtOAc = 2/1) |
| 134²⁾ | ⁱPr | H | F | 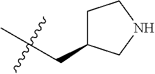 [SMILES: *C[C@H]1CCNC1] | tert-butyl (3R)-3- ({3-[7-fluoro-3- (propan-2-yl)-1H- indazol-1-yl]- 1,2,4-oxadiazol-5- yl}methyl)pyrrolidine- 1-carboxylate | LC-MS, m/z; 430 [M + H]+ |
| 135²⁾ | ⁱPr | H | F | 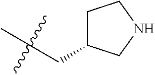 [SMILES: *C[C@@H]1CCNC1] | tert-butyl (3S)-3- ({3-[7-fluoro-3- (propan-2-yl)-1H- indazol-1-yl]- 1,2,4-oxadiazol-5- yl}methyl)pyrrolidine- 1-carboxylate | LC-MS, m/z; 430 [M + H]+ |
| 136²⁾ | Et | H | F | | tert-butyl (3S)-3- | TLC Rf = 0.14 |
| | | | | | {[3-(3-ethyl-7- | (hexane/EtOAc = |
| | | | | | fluoro-1H-indazol- | 4/1) |
| | | | | | 1-yl)-1,2,4- | |
| | | | | | oxadiazol-5- | |
| | | | | | yl]methyl}pyrrolidine- | |
| | | | | | 1-carboxylate | |
| 137²⁾ | ⁱPr | F | H | | tert-butyl (3S)-3- | TLC Rf = 0.17 |
| | | | | | ({3-[6-fluoro-3- | (hexane/EtOAc = |
| | | | | | (propan-2-yl)-1H- | 4/1) |
| | | | | | indazol-1-yl]- | |
| | | | | | 1,2,4-oxadiazol-5- | |
| | | | | | yl}methyl)pyrrolidine- | |
| | | | | | 1-carboxylate | |

¹⁾Prepared in the same manner as in Reference Example 044.
²⁾Prepared in the same manner as in Reference Example 033.
³⁾Prepared by treating isopropyl chloroformate as a condensing agent, and then using the same process as in Reference Example 60.

### Reference Example 138

### Preparation of cis-3-{[(2-nitrophenyl)sulfonyl]amino}cyclobutanecarboxylic acid

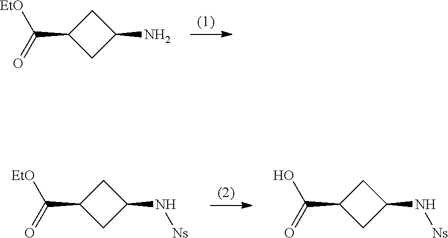

(1) To ethyl cis-3-aminocyclobutanecarboxylate (5 g, prepared according to the method described in WO 2009/060278) and triethylamine (1 ml) in dichloromethane (20 ml) was gradually added 2-nitrobenzenesulfonyl chloride (6.8 g), and the mixture was stirred at room temperature for 1 hour. To the reaction solution was added water (20 ml), and the resultant was extracted with dichloromethane (10 ml, ×2). The organic layer was dried over anhydrous magnesium sulfate and filtered, the filtrate was removed under reduced pressure, and the resultant was recrystallized from a mixture of hexane and ethyl acetate to give ethyl cis-3-{([2-(nitrophenyl)sulfonyl]amino}cyclobutanecarboxylate (8 g).

(2) To ethyl cis-3-{[2-(nitrophenyl)sulfonyl]amino}cyclobutanecarboxylate (5 g) in ethanol (30 ml) was added 2 mol/L sodium hydroxide (20 ml), and the mixture was stirred at room temperature for 3 hours. The reaction solution was adjusted to pH 2 by adding 1 mol/L HCl, and ethanol was removed under reduced pressure. The precipitated solid was collected on a filter, washed with water, and dried under reduced pressure to give the title compound (4.6 g).

¹H-NMR (DMSO-d₆) δ: 1.98-2.10 (2H, m), 2.15-2.27 (2H, m), 2.55-2.69 (1H, m), 3.58-3.74 (1H, m), 7.80-7.90 (2H, m), 7.91-8.01 (2H, m), 8.50 (1H, d, J=8.8 Hz), 12.15 (1H, s).

### Reference Example 139

### Preparation of 3-{3-[7-fluoro-3-(propan-2-yl)-1H-indazol-1-yl]-1,2,4-oxadiazol-5-yl}cyclobutanone

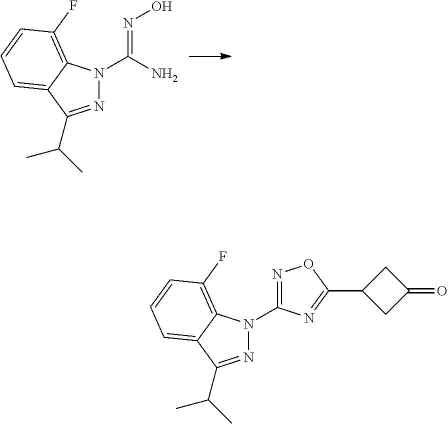 [SMILES: CC(C)c1nn(/C(N)=N/O)c2c(F)cccc12 ; CC(C)c1nn(-c2noc(C3CC(=O)C3)n2)c2c(F)cccc12]

The title compound was prepared in the same manner as in Reference Example 060 except that the 3-ethyl-6-fluoro-N-hydroxy-1H-indazole-1-carboximidamide was replaced with 7-fluoro-N′-hydroxy-3-(propan-2-yl)-1H-indazole-1-carboximidamide.

LC-MS, m/z; 315 [M+H]+

### Reference Example 140

### Preparation of 4-{3-[7-fluoro-3-(propan-2-yl)-1H-indazol-1-yl]-1,2,4-oxadiazol-5-yl}cyclohexanone

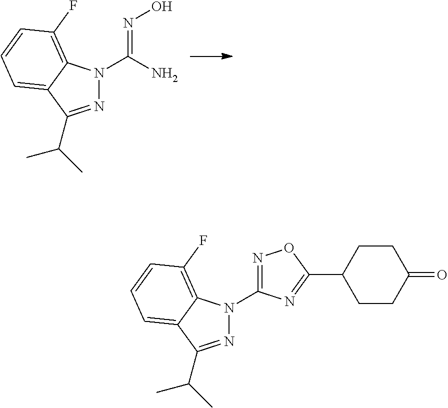 [SMILES: CC(C)c1nn(/C(N)=N/O)c2c(F)cccc12 ; CC(C)c1nn(-c2noc(C3CCC(=O)CC3)n2)c2c(F)cccc12]

The title compound was prepared in the same manner as in Reference Example 060 except that the 3-ethyl-6-fluoro-N-hydroxy-1H-indazole-1-carboximidamide and 3-oxocyclobutanecarboxylic acid were replaced with 7-fluoro-N′-hydroxy-3-(propan-2-yl)-1H-indazole-1-carboximidamide and 4-oxocyclohexane carboxylic acid, respectively.

### Reference Example 141

### Preparation of 7-fluoro-1-[1-(piperidin-4-yl)-1H-1,2,3-triazol-4-yl]-3-(propan-2-yl)-1H-indazole trifluoroacetate

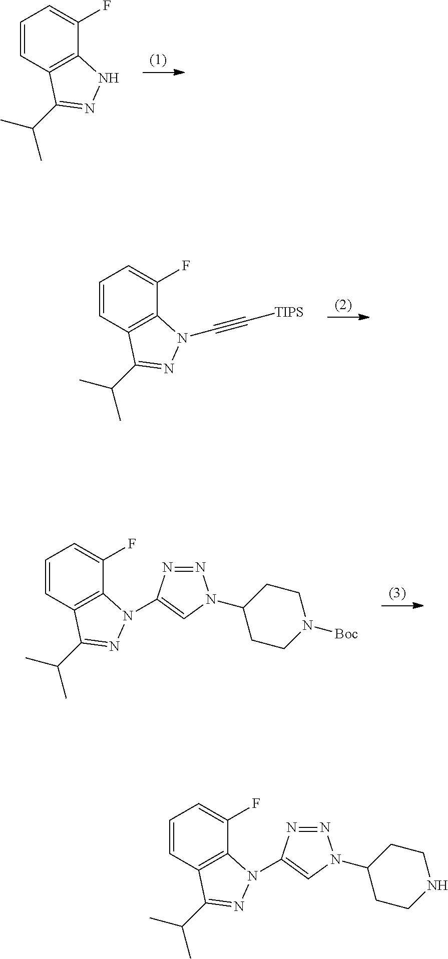

(1) 7-Fluoro-3-isopropyl-1H-indazole (712 mg), sodium carbonate (212 mg), pyridine (158 mg) and copper (II) chloride (59 mg) were suspended in toluene (5.0 ml). To the suspension was added dropwise triisopropylsilylacetylene (182 mg) in toluene (5.0 ml) in air at 70° C. over 3.5 hours, and then the mixture was stirred for 4 hours. The reaction mixture was concentrated under reduced pressure and the residue was purified by silica-gel chromatography (column; Hi-Flash™, developing solvent: hexane/ethyl acetate) to give 7-fluoro-3-(propan-2-yl)-1-[(tripropan-2-ylsilyl)ethynyl]-1H-indazole (53 mg).

(2) 7-Fluoro-3-(propan-2-yl)-1-[(tripropan-2-ylsilyl)ethynyl]-1H-indazole (53 mg) was dissolved in THF (2.6 ml). To the solution was added tetra-n-butylammonium fluoride (1M in THF, 0.18 ml), and the mixed solution was stirred at room temperature for 30 minutes. The reaction solution was concentrated under reduced pressure and the residue was purified by silica-gel chromatography (column; Hi-Flash™, developing solvent: hexane/ethyl acetate) to give 1-ethynyl-7-fluoro-3-isopropyl-1H-indazole (27 mg). Then, the obtained 1-ethynyl-7-fluoro-3-isopropyl-1H-indazole (27 mg) was mixed with tert-butyl 4-azidopiperidine-1-carboxylate (34 mg), copper (1.4 mg), and copper sulfate pentahydrate (1.7 mg) in a mixed solvent of tert-butylalcohol (1.4 ml) and water (1.4 ml). The mixture was stirred at 110° C. for 30 minutes under nitrogen atmosphere. To the reaction mixture was added brine, and the mixture was extracted with chloroform. The organic layer was dried over anhydrous sodium sulfate and concentrated under reduced pressure, and the residue was purified by silica-gel chromatography (column; Hi-Flash™, developing solvent: hexane:acetone) to give tert-butyl 4-{4-[7-fluoro-3-(propan-2-yl)-1H-indazol-1-yl]-1H-1,2,3-triazol-1-yl}piperidine-1-carboxylate (48 mg).

(3) Tert-butyl 4-{4-[7-fluoro-3-(propan-2-yl)-1H-indazol-1-yl]-1H-1,2,3-triazol-1-yl}piperidine-1-carboxylate (48 mg) was dissolved in methylene chloride (4.0 ml). To the solution was added trifluoroacetic acid (1.0 ml), and the mixture was stirred at room temperature for 1 hour.

The reaction solution was concentrated under reduced pressure to give a quantitative amount of the title compound.

LC-MS, m/z; 329 [M+H]+

### Reference Examples 142 to 143

### Preparation of 7-fluoro-1-[5-(piperidin-4-yl)-1,3-thiazol-2-yl]-3-(propan-2-yl)-1H-indazole trifluoroacetate and 7-fluoro-1-[2-(piperidin-4-yl)-1,3-thiazol-5-yl]-3-(propan-2-yl)-1H-indazole trifluoroacetate

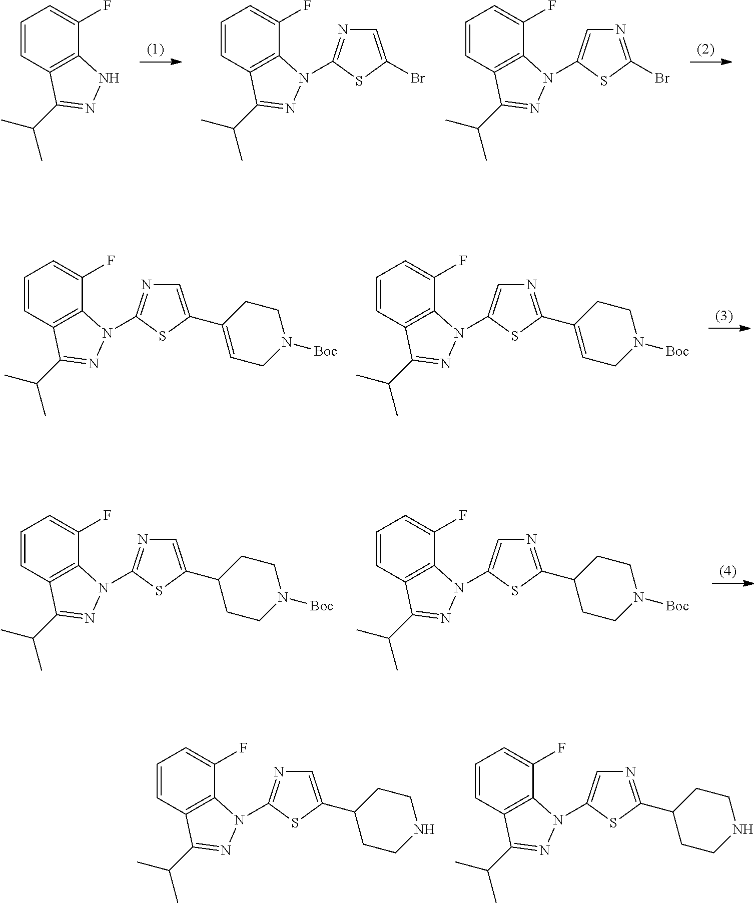

(1) 7-Fluoro-3-isopropyl-1H-indazole was dissolved in DMF (8.9 ml). To the solution was added 55% sodium hydride (262 mg) at ice temperature, and the mixture was stirred at ice temperature for 15 minutes. Then, 2,5-dibromothiazole (1.46 g) was added thereto, and the mixture was heated to 60° C. and stirred for 5 hours. To the reaction solution was added water, and the mixture was extracted with ethyl acetate. The organic layer was washed with water and brine, dried over anhydrous sodium sulfate, and concentrated under reduced pressure. The residue was purified by octadecyl-silica-gel chromatography (column; Hi-Flash™, developing solvent: acetonitrile/water) to give a mixture of 1-(5-bromo-1,3-thiazol-2-yl)-7-fluoro-3-(propan-2-yl)-1H-indazole and 1-(2-bromo-1,3-thiazol-5-yl)-7-fluoro-3-(propan-2-yl)-1H-indazole (743 mg).

(2) The mixture of 1-(5-bromo-1,3-thiazol-2-yl)-7-fluoro-3-(propan-2-yl)-1H-indazole and 1-(2-bromo-1,3-thiazol-5-yl)-7-fluoro-3-(propan-2-yl)-1H-indazole (170 mg), 1-Boc-1,2,5,6-tetrahydropyridine-4-boronic acid pinacol ester (186 mg), tetrakis(triphenylphosphine)palladium (0) (29 mg), and sodium carbonate (106 mg) were mixed in a solvent of water (1 ml) and DMF (4.2 ml). The mixture was stirred at 70° C. for 1 hour under nitrogen atmosphere. To the reaction mixture was added water, and the mixture was extracted with a mixed solvent of ethyl acetate/toluene. The organic layer was washed with water and saline, dried over anhydrous sodium sulfate, concentrated under reduced pressure, and the residue was purified by silica-gel chromatography (column; Hi-Flash™, developing solvent: hexane/ethyl acetate) to give a mixture of tert-butyl 4-{2-[7-fluoro-3-(propan-2-yl)-1H-indazol-1-yl]-1,3-thiazol-5-yl}-3,6-dihydropyridine-1 (2H)-carboxylate and tert-butyl 4-{5-[7-fluoro-3-(propan-2-yl)-1H-indazol-1-yl]-1,3-thiazol-2-yl}-3,6-dihydropyridine-1 (2H)-carboxylate (212 mg).

(3) The mixture of tert-butyl 4-{2-[7-fluoro-3-(propan-2-yl)-1H-indazol-1-yl]-1,3-thiazol-5-yl}-3,6-dihydropyridine-1 (2H)-carboxylate and tert-butyl 4-{5-[7-fluoro-3-(propan-2-yl)-1,3-indazol-1-yl]-1,3-thiazol-2-yl}-3,6-dihydropyridine-1 (2H)-carboxylate (100 mg) and 5% palladium carbon (20 mg) was mixed in ethyl acetate (3.0 ml), and the mixture was stirred at room temperature for 6 hours under hydrogen atmosphere (normal pressure). To the mixture was added 10% palladium carbon (50 mg), and the resultant mixture was further stirred at room temperature for 16 hours under hydrogen atmosphere (normal pressure). Then, the reaction mixture was filtered through Celite, the filtrate was concentrated under reduced pressure, and the residue was purified by silica-gel chromatography (column; Hi-Flash™, developing solvent: hexane/ethyl acetate) to give tert-butyl 4-{2-[7-fluoro-3-(propan-2-yl)-1H-indazol-1-yl]-1,3-thiazol-5-yl}piperidine-1-carboxylate (56 mg) and tert-butyl 4-{5-[7-fluoro-3-(propan-2-yl)-1H-indazol-1-yl]-1,3-thiazol-2-yl}piperidine-1-carboxylate (21 mg).

(4) Each of the tert-butyl 4-{2-[7-fluoro-3-(propan-2-yl)-1H-indazol-1-yl]-1,3-thiazol-5-yl}piperidine-1-carboxylate (56 mg) and the tert-butyl 4-{5-[7-fluoro-3-(propan-2-yl)-1H-indazol-1-yl]-1,3-thiazol-2-yl}piperidine-1-carboxylate (21 mg) was dissolved in methylene chloride, and trifluoroacetic acid was added thereto. Each of the mixed solutions was stirred at room temperature. After confirming the completion of the reactions, each of the reaction mixtures was concentrated to give a quantitative amount of the two title compounds.

LC-MS, m/z; 345 [M+H]+

### Reference Example 144

### Preparation of 7-fluoro-1-[5-(piperidin-4-yl)-1H-imidazol-2-yl]-3-(propan-2-yl)-1H-indazole

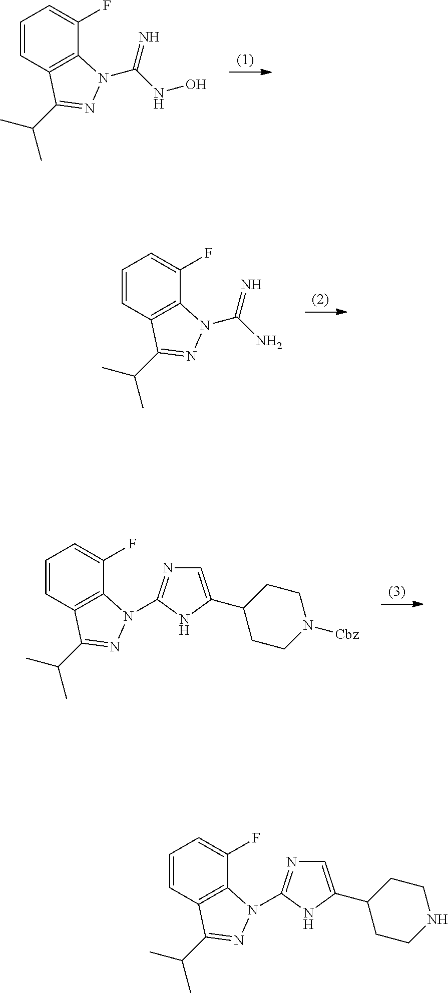 [SMILES: CC(C)c1nn(C(=N)NO)c2c(F)cccc12 ; CC(C)c1nn(C(=N)N)c2c(F)cccc12 ; CC(C)c1nn(-c2ncc(C3CCN(C(=O)OCc4ccccc4)CC3)[nH]2)c2c(F)cccc12 ; CC(C)c1nn(-c2ncc(C3CCNCC3)[nH]2)c2c(F)cccc12]

(1) 7-Fluoro-N′-hydroxy-3-(propan-2-yl)-1H-indazole-1-carboximidamide (236 mg), acetic anhydride (112 mg) and 5% palladium carbon (100 mg) were mixed in acetic acid (23 ml), and the mixture was stirred at room temperature for 5 hours under hydrogen atmosphere (normal pressure). The reaction mixture was filtered through Celite, the filtrate was concentrated under reduced pressure, and the residue was purified by size exclusion column chromatography (Moving bed: chloroform) to give 7-fluoro-3-(propan-2-yl)-1H-indazole-1-carboximidamide (100 mg).

(2) The 7-fluoro-3-(propan-2-yl)-1H-indazole-1-carboximidamide (55 mg), benzyl 4-(2-bromoacetyl)piperidine-1-carboxylate (85 mg) and potassium carbonate (159 mg) were mixed in DMF (1.2 ml), and the mixture was stirred at room temperature for 19 hours. To the reaction mixture was added water, and the resultant was extracted with a mixed solvent of ethyl acetate/toluene. The organic layer was washed with water and brine, dried over anhydrous sodium sulfate, and concentrated. The residue was purified by silica-gel chromatography (column; Hi-Flash™, developing solvent: hexane/ethyl acetate) to give benzyl 4-{2-[7-fluoro-3-(propan-2-yl)-1H-indazol-1-yl]-1H-imidazol-5-yl}piperidine-1-carboxylate (61 mg).

(3) The benzyl 4-{2-[7-fluoro-3-(propan-2-yl)-1H-indazol-1-yl]-1H-imidazol-5-yl}piperidine-1-carboxylate (61 mg) and 5% palladium carbon (13 mg) were mixed in ethyl acetate (6.1 ml), and the mixture was stirred at room temperature for 2 hours under hydrogen atmosphere (normal pressure). To the mixture was further added 10% palladium carbon (30 mg), and the resultant mixture was stirred at room temperature for 19 hours under hydrogen atmosphere (normal pressure). The reaction mixture was filtered through Celite, and the filtrate was concentrated under reduced pressure to give the title compound (30 mg).

LC-MS, m/z; 328 [M+H]+

### Reference Example 145

### Preparation of cis-1-(tert-butoxycarbonyl)-3-methoxypiperidine-4-carboxylic acid

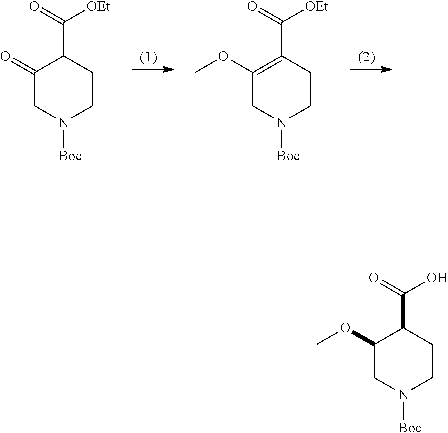 [SMILES: CCOC(=O)C1CCN(C(=O)OC(C)(C)C)CC1=O ; CCOC(=O)C1=C(OC)CN(C(=O)OC(C)(C)C)CC1 ; CO[C@@H]1CN(C(=O)OC(C)(C)C)CC[C@@H]1C(=O)O]

(1) To 1-tert-butyl 4-ethyl 3-oxopiperidine-1,4-dicarboxylate (4.9 g) of tetrahydrofuran (50 ml) was gradually added 60% sodium hydride (1.1 g). The mixture was stirred at room temperature for 1 hour. To the reaction solution was added dimethyl sulfate (2.5 ml), and the mixture was stirred at 60° C. for 3 hours. The reaction solution was cooled to room temperature. To the solution was added saturated sodium hydrogen carbonate aqueous solution (50 ml), and the mixture was extracted with ethyl acetate (20 ml, ×3). The organic layer was dried over anhydrous sodium sulfate, filtered, and concentrated under reduced pressure. The residue was purified by silica-gel chromatography to give 1-tert-butyl 4-ethyl 3-methoxy-5,6-dihydropyridine-1,4(2H-dicarboxylate (2.4 g).

¹H-NMR (CDCl₃) δ: 1.20-1.28 (3H, m), 1.45 (9H, d, J=0.6 Hz), 2.34-2.44 (2H, m), 3.36-3.45 (2H, m), 3.75 (3H, s), 4.02-4.22 (4H, m).

LC-MS, m/z; 286 [M+H]+

(2) To the 1-tert-butyl 4-ethyl 3-methoxy-5,6-dihydropyridine-1,4(2H)-dicarboxylate (2.4 g) in ethanol (20 ml) was added 10% palladium carbon (300 mg). The mixture was stirred at room temperature for 1 hour under hydrogen atmosphere. The reaction solution was filtered through Celite. To the filtrate was added 2 mol/L aqueous sodium hydroxide (15 ml), and the mixture was stirred for 3 hours. The reaction solution was adjusted to pH 2 with 1 mol/L HCl, ethanol was removed under reduced pressure, the aqueous layer was extracted with ethyl acetate (10 m1, x3). The organic layer was dried over anhydrous sodium sulfate, filtered and concentrated under reduced pressure, and the residue was recrystallized from diethylether to give the title compound (830 mg).

¹H-NMR (CDCl₃) δ: 1.44 (9H, s), 1.67 (1H, d, J=14.7 Hz), 2.00 (1H, ddd, J=25.2, 11.7, 4.3 Hz), 2.55-2.64 (1H, m), 2.66-2.94 (2H, m), 3.40 (3H, s), 3.67-3.76 (1H, m), 3.84-4.21 (1H, m), 4.24-4.32 (1H, m).

LC-MS, m/z; 260 [M+H]+

The compounds in the following table (i.e. Reference Examples 146 to 149) were prepared in the same manner as in Reference Example 033 or Reference Example 060 except that the 3-ethyl-6-fluoro-N′-hydroxy-1H-indazole-1-carboximidamide of Reference Example 033 and Reference Example 060 was replaced with the corresponding starting compound and carboxylic acid.

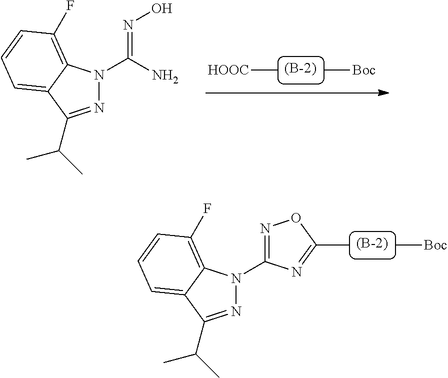

Wherein (B-2) means each cyclic amino structure shown in the following table; and the Boc group is attached to the nitrogen atom in the cyclic amine of (B-2).

TABLE 12

| Ref. | | Compound | ¹H-NMR/LC-MS, |
|---|---|---|---|
| Ex. | (B-2) | Name | m/z |
| 146¹⁾ | 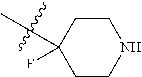 [SMILES: *C1(F)CCNCC1] | tert-butyl 4-fluoro- 4-{3-[7- fluoro-3-(propan-2- yl)-1H- indazol-1-yl]-1,2,4- oxadiazol- 5-yl}piperidine-1- carboxylate | LC-MS, m/z; 448 [M + H]+ |
| 147¹⁾ | 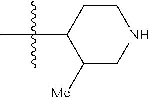 [SMILES: *C1CCNCC1C] | tert-butyl 4-{3-[7- fluoro-3- (propan-2-yl)-1H- indazol-1- yl]-1,2,4-oxadiazol- 5-yl}-3- methylpiperidine-1- carboxylate | LC-MS, m/z; 444 [M + H]+ |
| 148²⁾ | 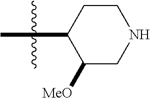 [SMILES: *[C@H]1CCNC[C@H]1OC] | tert-butyl (3S,4S)-4- {3-[7- fluoro-3-(propan-2- yl)-1H- indazol-1-yl]-1,2,4- oxadiazol- 5-yl}-3- methoxypiperidine- 1- carboxylate and tert-butyl (3R,4R)- 4-{3-[7- fluoro-3-(propan-2- yl)-1H- indazol-1-yl]-1,2,4- oxadiazol- 5-yl}-3- methoxypiperidine- 1- carboxylate | LC-MS, m/z; 460 [M + H]+ |
| 149¹⁾ | 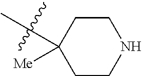 [SMILES: *C1(C)CCNCC1] | tert-butyl 4-{3-[7- fluoro-3- (propan-2-yl)-1H- indazol-1- yl]-1,2,4-oxadiazol- 5-yl}-4- methylpiperidine-1- carboxylate | LC-MS, m/z; 444 [M + H]+ |

¹⁾In the same manner as in Reference Example 033.
²⁾In the same manner as in Reference Example 060.

### Reference Example 150

### Preparation of 7-fluoro-N′-hydroxy-3-methoxy-1H-indazole-1-carboximidamide

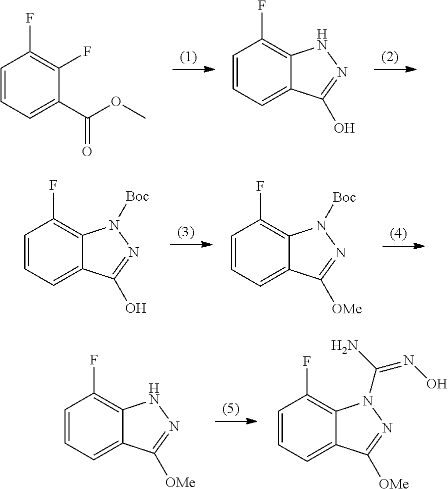

(1) Methyl 2,3-difluorobenzoate (2.00 g) and hydrazine monohydrate (2.91 g) was mixed in 1,4-dioxane (40 ml), and the mixture was heated at 100° C. for 19 hours. To the reaction mixture was added silica-gel, and the mixture was concentrated. The residue was purified by silica-gel chromatography (column; Hi-Flash™, developing solvent: chloroform/methanol) to give 7-fluoro-1H-indazol-3-ol (1.73 g).

(2) The 7-fluoro-1H-indazol-3-ol (1.68 g) and DMAP (67 mg) was mixed in acetonitrile (17 ml), and to the mixture was added dropwise di-tert-butyl dicarbonate (2.53 g) in acetonitrile (17 ml) at room temperature. The reaction mixture was stirred at room temperature for 5 hours, and concentrated under reduced pressure. The residue was purified by silica-gel chromatography (column; Hi-Flash™, developing solvent: chloroform/methanol) and further washed with ethyl acetate to give tert-butyl 7-fluoro-3-hydroxy-1H-indazole-1-carboxylate (1.82 g).

(3) The tert-butyl 7-fluoro-3-hydroxy-1H-indazole-1-carboxylate (126 mg), methyl iodide (255 mg) and silver carbonate (489 mg) were mixed in acetonitrile (2.5 ml), and the mixture was stirred at 80° C. for 4 hours. The residue was purified by silica-gel chromatography (column; Hi-Flash™, developing solvent: hexane/ethyl acetate) to give tert-butyl 7-fluoro-3-methoxy-1H-indazole-1-carboxylate (105 mg).

(4) Tert-butyl 7-fluoro-3-methoxy-1H-indazole-1-carboxylate (105 mg) was added to 4 N HCl (in 1,4-dioxane), and the mixture was stirred at room temperature for 16 hours. The reaction mixture was concentrated under reduced pressure to give a quantitative amount of 7-fluoro-3-methoxy-1H-indazole.

(5) The title compound was prepared in the same manner as in Reference Example 016 except that the 3-ethyl-6-fluoro-1H-indazole was replaced with the above 7-fluoro-3-methoxy-1H-indazole.

TLC Rf=0.52 (CHCl₃/MeOH=20/1)

### Example 001

### Preparation of 3-ethyl-6-fluoro-1-[5-(piperidin-4-yl)-1,2,4-oxadiazol-3-yl]-1H-indazole trifluoroacetate

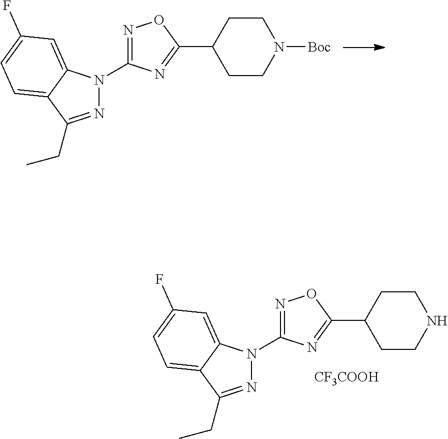 [SMILES: CCc1nn(-c2noc(C3CCN(C(=O)OC(C)(C)C)CC3)n2)c2cc(F)ccc12 ; CCc1nn(-c2noc(C3CCNCC3)n2)c2cc(F)ccc12 ; "CF3COOH"]

Tert-butyl 4-[3-(3-ethyl-6-fluoro-1H-indazol-1-yl)-1,2,4-oxadiazol-5-yl]piperidine-1-carboxylate (1.66 g) was dissolved in dichloromethane (5.0 ml). To the solution was added trifluoroacetic acid (2.0 ml), and the mixture was stirred at room temperature for 30 minutes. The reaction solution was evaporated under reduced pressure, and the residue was crystallized by adding diethyl ether (20 ml) thereto. The resultant crystal was collected on a filter to give the title compound (1.56 g) as a white solid.

LC-MS, m/z; 316 [M+H]+

The compounds in the following table (i.e. Examples 002 to 011) were prepared in the same manner as in Example 001 except that the tert-butyl 4-[3-(3-ethyl-6-fluoro-1H-indazol-1-yl)-1,2,4-oxadiazol-5-yl]piperidine-1-carboxylate was replaced with the corresponding starting compound (which is described in Reference Examples 033 to 049).

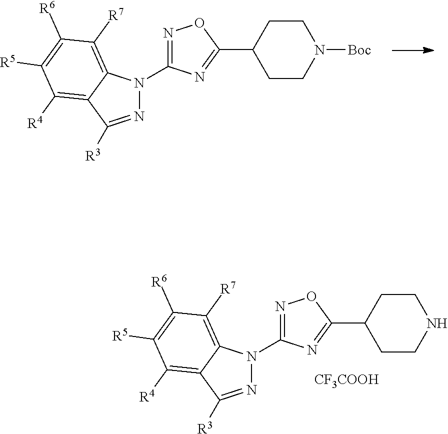

TABLE 13

| | | | | | | Com- | |
|---|---|---|---|---|---|---|---|
| | | | | | | pound | LC-MS, |
| Ex. | R³ | R⁴ | R⁵ | R⁶ | R⁷ | Name | m/z |
| 002 | Et | F | H | H | H | 3-ethyl-4- | LC-MS, |
| | | | | | | fluoro- | m/z; |
| | | | | | | 1-[5- | 316 |
| | | | | | | (piperidin- | [M + H]+ |
| | | | | | | 4- | |
| | | | | | | yl)-1,2,4- | |
| | | | | | | oxadiazol- | |
| | | | | | | 3-yl]- | |
| | | | | | | 1H- | |
| | | | | | | indazole | |
| | | | | | | trifluoroacetate | |
| 003 | Et | H | F | H | H | 3-ethyl-5-fluoro- | LC-MS, |
| | | | | | | 1-[5-(piperidin- | m/z; |
| | | | | | | 4- | 316 |
| | | | | | | yl)-1,2,4- | [M + H]+ |
| | | | | | | oxadiazol-3-yl]- | |
| | | | | | | 1H-indazole | |
| | | | | | | trifluoroacetate | |
| 004 | Et | H | H | H | F | 3-ethyl-7- | LC-MS, |
| | | | | | | fluoro- | m/z; |
| | | | | | | 1-[5-(piperidin- | 316 |
| | | | | | | 4- | [M + H]+ |
| | | | | | | yl)-1,2,4- | |
| | | | | | | oxadiazol-3-yl]- | |
| | | | | | | 1H-indazole | |
| | | | | | | trifluoroacetate | |
| 005 | 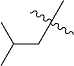 [SMILES: *CC(C)C] | H | H | H | H | 3-(2- methylpropyl)- 1- [5-(piperidin-4- yl)-1,2,4- oxadiazol-3-yl]- 1H-indazole trifluoroacetate | LC-MS, m/z; 326 [M + H]+ |
| 006 | ⁱPr | H | H | H | F | 7-fluoro-1-[5- | LC-MS, |
| | | | | | | (piperidin-4-yl)- | m/z; |
| | | | | | | 1,2,4-oxadiazol- | 330 |
| | | | | | | 3- | [M + H]+ |
| | | | | | | yl]-3-(propan-2- | |
| | | | | | | yl)-1H-indazole | |
| | | | | | | trifluoroacetate | |
| 007 | Et | H | H | MeO | H | 3-ethyl-6- | LC-MS, |
| | | | | | | methoxy- | m/z; |
| | | | | | | 1-[5-(piperidin- | 328 |
| | | | | | | 4- | [M + H]+ |
| | | | | | | yl)-1,2,4- | |
| | | | | | | oxadiazol-3-yl]- | |
| | | | | | | 1H-indazole | |
| | | | | | | trifluoroacetate | |
| 008 | 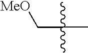 [SMILES: *COC] | H | H | H | H | 3- (methoxymethyl)- 1-[5-(piperidin-4- yl)-1,2,4- oxadiazol-3-yl]- 1H-indazole trifluoroacetate | LC-MS, m/z; 314 [M + H]+ |
| 009 | 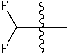 [SMILES: *C(F)F] | H | H | H | H | 3- (difluoromethyl)- 1-[5-(piperidin-4- yl)-1,2,4- oxadiazol-3-yl]- 1H-indazole trifluoroacetate | LC-MS, m/z; 320 [M + H]+ |
| 010 | Br | H | H | H | H | 3-bromo-1-[5- | LC-MS, |
| | | | | | | (piperidin-4-yl)- | m/z; |
| | | | | | | 1,2,4-oxadiazol- | 348 |
| | | | | | | 3- | [M + H]+ |
| | | | | | | yl]-1H-indazole | |
| | | | | | | trifluoroacetate | |
| 011 | Et | H | H | H | H | 3-ethyl-1-[5- | LC-MS, |
| | | | | | | (piperidin-4-yl)- | m/z; |
| | | | | | | 1,2,4-oxadiazol- | 298 |
| | | | | | | 3- | [M + H]+ |
| | | | | | | yl]-1H-indazole | |
| | | | | | | trifluoroacetate | |

### Example 012

### Preparation of 1-[5-(piperidin-4-yl)-1,2,4-oxadiazol-3-yl]-3-(propan-2-yl)-1H-indazole hydrochloride

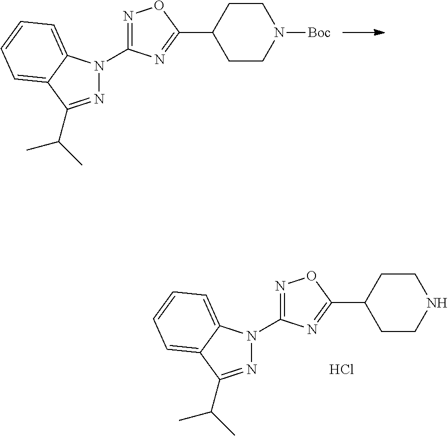 [SMILES: CC(C)c1nn(-c2noc(C3CCN(C(=O)OC(C)(C)C)CC3)n2)c2ccccc12 ; CC(C)c1nn(-c2noc(C3CCNCC3)n2)c2ccccc12 ; Cl]

To tert-butyl 4-{3-[3-(propan-2-yl)-1H-indazol-1-yl]-1,2,4-oxadiazol-5-yl}piperidine-1-carboxylate (0.64 g) was added 4 N HCl/1,4-dioxane (15 ml), and the mixture was stirred at room temperature for 30 minutes. The crystallized solid was collected on a filter, washed with hexane, dried at 60° C. under reduced pressure to give the title compound (0.40 g) as a white solid.

LC-MS, m/z; 312 [M+H]+

The compounds in the following table (i.e. Examples 013 to 019) were prepared in the same manner as in Example 012 except that the tert-butyl 4-{3-[3-(propan-2-yl)-11′-indazol-1-yl]-1,2,4-oxadiazol-5-yl}piperidine-1-carboxylate was replaced with the corresponding starting compound (which is described in Reference Examples 033 to 049).

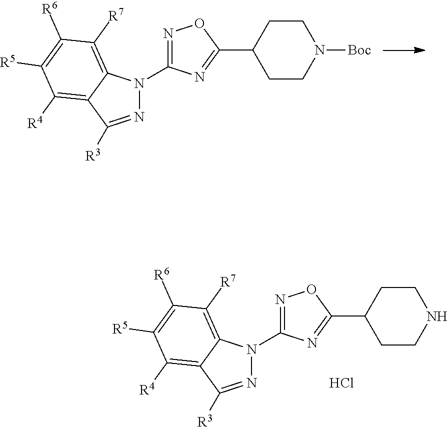

TABLE 14

| | | | | | | Compound | ¹H-NMR/ |
|---|---|---|---|---|---|---|---|
| Ex. | R³ | R⁴ | R⁵ | R⁶ | R⁷ | Name | LC-MS, m/z |
| 013 | Et | H | H | H | H | 3-ethyl-1-[5- | LC-MS, m/z; |
| | | | | | | (piperidin-4- | 298 [M + H]+ |
| | | | | | | yl)-1,2,4- | |
| | | | | | | oxadiazol-3- | |
| | | | | | | yl]-1H-indazole | |
| | | | | | | hydrochloride | |
| 014 | 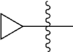 [SMILES: *C1CC1] | H | H | H | H | 3-cyclopropyl- 1-[5- (piperidin-4- yl)-1,2,4- oxadiazol-3- yl]-1H-indazole hydrochloride | ¹H-NMR (400 MHz, CDCl₃): δ 0.85-0.97 (m, 2H), 0.98-1.09 (m, 2H), 2.06-2.35 (m, 5H), 2.89- 3.01 (m, 2H), 3.15-3.28 (m, 2H), 7.11 (t, J = 7.4 Hz, 1H), 7.34 (t, J = 7.6 Hz, 1H), 7.58 (d, J = 8.0 Hz, 1H), 8.20 (d, J = 8.8 Hz, 1H). |
| 015 | Me | H | H | H | H | 3-methyl-1-[5- | LC-MS, m/z; |
| | | | | | | (piperidin-4- | 284 [M + H]+ |
| | | | | | | yl)-1,2,4- | |
| | | | | | | oxadiazol-3- | |
| | | | | | | yl]-1H-indazole | |
| | | | | | | hydrochloride | |
| 016 | 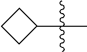 [SMILES: *C1CCC1] | H | H | H | H | 3-cyclobutyl-1- [5-(piperidin- 4-yl)-1,2,4- oxadiazol-3- yl]-1H-indazole hydrochloride | No data |
| 017 | Cl | H | H | H | H | 3-chloro-1-[5- | LC-MS, m/z; |
| | | | | | | (piperidin-4- | 304 [M + H]+ |
| | | | | | | yl)-1,2,4- | |
| | | | | | | oxadiazol-3- | |
| | | | | | | yl]-1H-indazole | |
| | | | | | | hydrochloride | |
| 018 | ⁱPr | H | H | H | F | 7-fluoro-1-[5- | LC-MS, m/z; |
| | | | | | | (piperidin-4- | 330 [M + H]+ |
| | | | | | | yl)-1,2,4- | |
| | | | | | | oxadiazol-3- | |
| | | | | | | yl]-3-(propan- | |
| | | | | | | 2-yl)-1H- | |
| | | | | | | indazole | |
| | | | | | | hydrochloride | |
| 019 | Et | H | H | H | Cl | 7-chloro-3- | LC-MS, m/z; |
| | | | | | | ethyl-1-[5- | 332 [M + H]+ |
| | | | | | | (piperidin-4- | |
| | | | | | | yl)-1,2,4- | |
| | | | | | | oxadiazol-3- | |
| | | | | | | yl]-1H-indazole | |
| | | | | | | hydrochloride | |

The compounds in the following table (i.e. Examples 020 to 022) were prepared in the same manner as in Example 001 or Example 012 except that the corresponding starting compound (which is described in Reference Examples 050 to 052) was used.

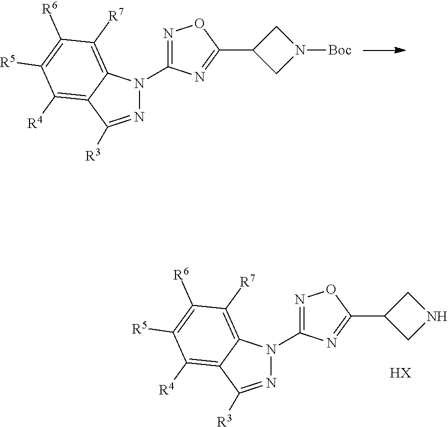

Wherein HX is hydrochloric acid or trifluoroacetic acid.

TABLE 15

| Ex. | R³ | R⁴ | R⁵ | R⁶ | R⁷ | Compound Name | LC-MS, m/z |
|---|---|---|---|---|---|---|---|
| 020 | Et | H | H | H | H | 1-[5-(azetidin-3-yl)- | LC-MS, m/z; |
| | | | | | | 1,2,4-oxadiazol-3-yl]- | 271 [M + H]+ |
| | | | | | | 3-ethyl-1H-indazole | |
| | | | | | | hydrochloride | |
| 021 | ⁱPr | H | H | H | H | 1-[5-(azetidin-3-yl)- | ¹H NMR (400 |
| | | | | | | 1,2,4-oxadiazol-3-yl]- | MHz, CDCl₃) δ |
| | | | | | | 3-(propan-2-yl)-1H- | 1.47 (s, 9 |
| | | | | | | indazole hydrochloride | H), 1.52 (d, |
| | | | | | | | 6 H), |
| | | | | | | | 3.53 (m, 1 H), |
| | | | | | | | 4.12 (dd, J = |
| | | | | | | | 7.6 Hz, |
| | | | | | | | 15.2 Hz, 1 |
| | | | | | | | H), 4.38 (d, |
| | | | | | | | 2 H), |
| | | | | | | | 4.40 (d, 2 H), |
| | | | | | | | 7.32 (t, J = |
| | | | | | | | 7.6 Hz, 1 |
| | | | | | | | H), 7.58 (t, |
| | | | | | | | J = 7.6 Hz, 1 |
| | | | | | | | H), 7.83 (d, |
| | | | | | | | J = 8.0 Hz, 1 |
| | | | | | | | H), 8.28 (d, |
| | | | | | | | J = 8.4 Hz, |
| | | | | | | | 1H). |
| 022 | Et | H | H | F | H | 1-[5-(azetidin-3-yl)- | LC MS, m/z; |
| | | | | | | 1,2,4-oxadiazol-3-yl]- | 288 [M + H]+ |
| | | | | | | 3-ethyl-6-fluoro-1H- | |
| | | | | | | indazole | |
| | | | | | | trifluoroacetate | |

### Example 023

### Preparation of tert-butyl 4-(2-{4-[3-(3-ethyl-1H-indazol-1-yl)-1,2,4-oxadiazol-5-yl]piperidin-1-yl}ethyl)piperidine-1-carboxylate

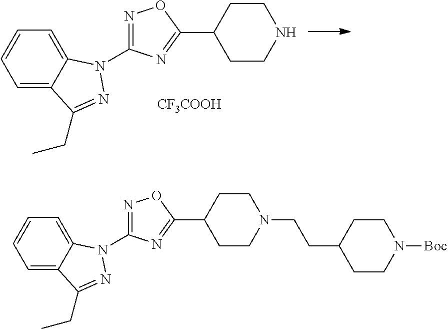 [SMILES: CCc1nn(-c2noc(C3CCNCC3)n2)c2ccccc12 ; "CF3COOH" ; CCc1nn(-c2noc(C3CCN(CCC4CCN(C(=O)OC(C)(C)C)CC4)CC3)n2)c2ccccc12]

3-Ethyl-1-[5-(piperidin-4-yl)-1,2,4-oxadiazol-3-yl]-1H-indazole trifluoroacetate (100 mg) was suspended in N,N-dimethylformamide (3 ml). To the suspension were added tert-butyl 4-(2-iodoethyl)piperidine-1-carboxylate (115 mg) and potassium carbonate (135 mg), and the mixture was refluxed overnight. The reaction solution was cooled to room temperature and water was added thereto. The mixture was extracted with ethyl acetate, the organic layer was washed with water and brine, dried over sodium sulfate and filtered, and the filtrate was concentrated under reduced pressure. The residue was purified by silica-gel chromatography (column; Hi-Flash™ Amino Column, developing solvent: hexane/ethyl acetate=2:1) to give the title compound (58 mg) as a white solid.

LC-MS, m/z; 509 [M+H]+

### Example 024

### Preparation of tert-butyl 4-({4-[3-(3-ethyl-6-fluoro-1H-indazol-1-yl)-1,2,4-oxadiazol-5-yl]piperidin-1-yl}methyl)piperidine-1-carboxylate

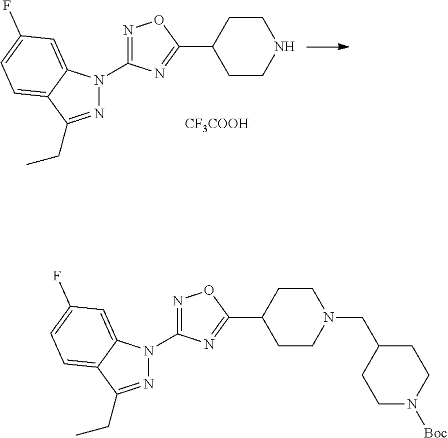 [SMILES: CCc1nn(-c2noc(C3CCNCC3)n2)c2cc(F)ccc12 ; "CF3COOH" ; CCc1nn(-c2noc(C3CCN(CC4CCN(C(=O)OC(C)(C)C)CC4)CC3)n2)c2cc(F)ccc12]

3-Ethyl-6-fluoro-1-[5-(piperidin-4-yl)-1,2,4-oxadiazol-3-yl]-1H-indazole trifluoroacetate (150 mg) was suspended in acetonitrile (4.00 ml). To the suspension were added potassium carbonate (290 mg), tert-butyl 4-(bromomethyl)piperidine-1-carboxylate (194 mg) and sodium iodide (58 mg), and the mixture was stirred under reflux overnight. The reaction solution was cooled to room temperature, water (20 ml) was added thereto, and the mixture was extracted with ethyl acetate (20 ml). The organic layer was washed with water (20 ml×2) again, and dried over sodium sulfate. The organic layer was evaporated under reduced pressure, and the residue was purified by silica-gel chromatography (column; Hi-Flash™ Amino Column, developing solvent: hexane/ethyl acetate=2/1) to give the title compound (160 mg) as a colorless oil.

LC-MS, m/z; 513 [M+H]+

The compounds in the following table (i.e. Examples 025 to 026) were prepared in the same manner as in Example 024 except that the 3-ethyl-6-fluoro-1-[5-(piperidin-4-yl)-1,2,4-oxadiazol-3-yl]-1H-indazole trifluoroacetate was replaced with the corresponding starting compound.

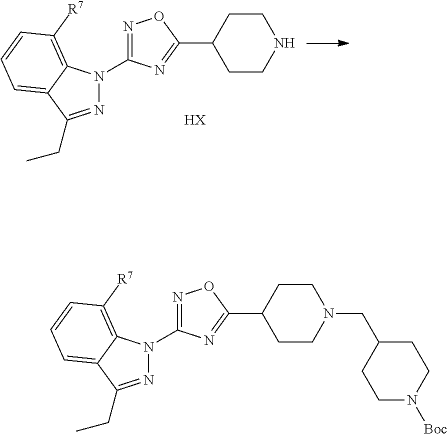

Wherein HX is hydrochloric acid or trifluoroacetic acid.

TABLE 16

| Ex. | R⁷ | Compound Name | LC-MS, m/z |
|---|---|---|---|
| 025 | H | tert-butyl 4-({4-[3-(3-ethyl-1H-indazol- | LC-MS, m/z; |
| | | 1-yl)-1,2,4-oxadiazol-5-yl]piperidin-1- | 495 [M + H]+ |
| | | yl}methyl)piperidine-1-carboxylate | |
| 026 | F | tert-butyl 4-({4-[3-(3-ethyl-7-fluoro-1H- | LC-MS, m/z; |
| | | indazol-1-yl)-1,2,4-oxadiazol-5- | 513 [M + H]+ |
| | | yl]piperidin-1-yl}methyl)piperidine-1- | |
| | | carboxylate | |

### Example 027

### Preparation of tert-butyl 4-[(4-{3-[7-fluoro-3-(propan-2-yl)-1H-indazol-1-yl]-1,2,4-oxadiazol-5-yl}piperidin-1-yl)methyl]piperidine-1-carboxylate

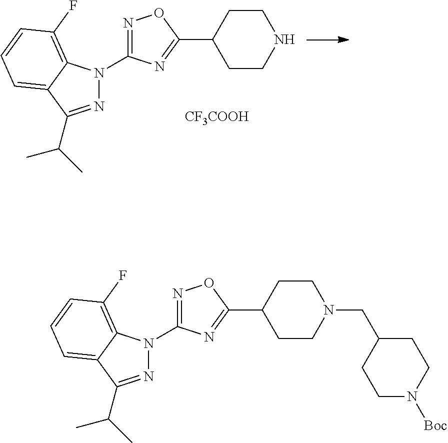 [SMILES: CC(C)c1nn(-c2noc(C3CCNCC3)n2)c2c(F)cccc12 ; "CF3COOH" ; CC(C)c1nn(-c2noc(C3CCN(CC4CCN(C(=O)OC(C)(C)C)CC4)CC3)n2)c2c(F)cccc12]

7-Fluoro-1-[5-(piperidin-4-yl)-1,2,4-oxadiazol-3-yl]-3-(propan-2-yl)-1H-indazole trifluoroacetate (1.43 g) was dissolved in dichloromethane (20 ml). To the solution were added 1-Boc-4-piperidine-carboxaldehyde (1.37 g) and triacetoxysodium borohydride (1.36 g), and the mixture was stirred at room temperature overnight. To the reaction mixture was added saturated sodium bicarbonate aqueous solution, and the mixture was extracted with chloroform. The organic layer was dried over sodium sulfate and filtered, and the filtrate was concentrated under reduced pressure. The residue was purified by silica-gel chromatography (column; Hi-Flash™ Amino Column, developing solvent: hexane/ethyl acetate=1/1) to give tert-butyl 4-[(4-{3-[7-fluoro-3-(propan-2-yl)-1H-indazole-1-yl]-1,2,4-oxadiazol-5-yl}piperidin-1-yl)methyl]piperidine-1-carboxylate (1.69 g) as a colorless oil.

LCMS, m/z; 527 [M+H]+

### Example 028

### Preparation of tert-butyl (2S)-2-({4-[3-(3-ethyl-1H-indazol-1-yl)-1,2,4-oxadiazol-5-yl]piperidin-1-yl}methyl)pyrrolidine-1-carboxylate

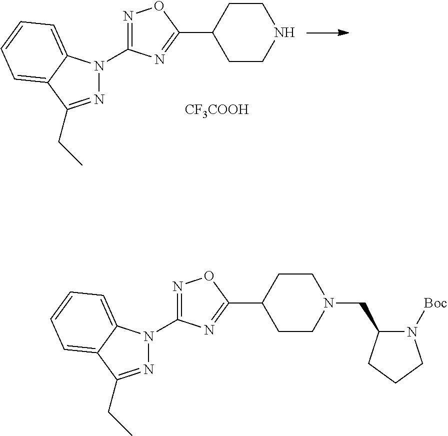 [SMILES: CCc1nn(-c2noc(C3CCNCC3)n2)c2ccccc12 ; "CF3COOH" ; CCc1nn(-c2noc(C3CCN(C[C@@H]4CCCN4C(=O)OC(C)(C)C)CC3)n2)c2ccccc12]

3-Ethyl-1-[5-(piperidin-4-yl)-1,2,4-oxadiazol-3-yl]-1H-indazole trifluoroacetate (100 mg) was dissolved in dichloromethane (5 ml). To the solution were added (S)-(−)-1-tert-butoxycarbonyl-2-pyrrolidinecarbaldehyde (73 mg) and triacetoxysodium borohydride (155 mg) at 0° C. with stirring, and the mixture was stirred at room temperature for 3 hours. To the reaction solution was added saturated sodium bicarbonate aqueous solution, and the mixture was extracted with ethyl acetate. The organic layer was washed with water and brine, dried over sodium sulfate, and concentrated under reduced pressure. The residue was purified by silica-gel chromatography (column; Hi-Flash™ Amino Column, developing solvent: hexane/ethyl acetate=1:1) to give the title compound (108 mg) as a white solid.

LC-MS, m/z; 481 [M+H]+

The compounds in the following table (i.e. Examples 029 to 032) were prepared in the same manner as in Example 028 except that the 3-ethyl-1-[5-(piperidin-4-yl)-1,2,4-oxadiazol-3-yl]-1H-indazole trifluoroacetate was replaced with the corresponding starting compound.

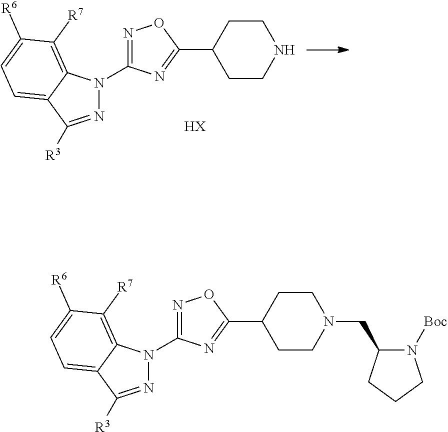

Wherein HX is hydrochloric acid or trifluoroacetic acid.

TABLE 17

| Ex. | R³ | R⁶ | R⁷ | Compound Name | LC-MS, m/z |
|---|---|---|---|---|---|
| 029 | Me | F | H | tert-butyl (2S)-2-({4-[3-(6- | LC-MS, |
| | | | | fluoro-3-methyl-1H-indazol-1- | m/z; |
| | | | | yl)-1,2,4-oxadiazol-5- | 485 [M + H]+ |
| | | | | yl]piperidin-1- | |
| | | | | yl}methyl)pyrrolidine-1- | |
| | | | | carboxylate | |
| 030 | Et | F | H | tert-butyl (2S)-2-({4-[3-(3- | LC-MS, |
| | | | | ethyl-6-fluoro-1H-indazol-1-yl)- | m/z; |
| | | | | 1,2,4-oxadiazol-5-yl]piperidin- | 499 [M + H]+ |
| | | | | 1-yl}methyl)pyrrolidine-1- | |
| | | | | carboxylate | |
| 031 | Et | H | F | tert-butyl (2S)-2-({4-[3-(3- | LC-MS, |
| | | | | ethyl-7-fluoro-1H-indazol-1-yl)- | m/z; |
| | | | | 1,2,4-oxadiazol-5-yl]piperidin- | 499 [M + H]+ |
| | | | | 1-yl}methyl)pyrrolidine-1- | |
| | | | | carboxylate | |
| 032 | ⁱPr | H | F | tert-butyl (2S)-2-[(4-{3-[7- | LC-MS, |
| | | | | fluoro-3-(propan-2-yl)-1H- | m/z; |
| | | | | indazol-1-yl]-1,2,4-oxadiazol-5- | 513 [M + H]+ |
| | | | | yl}piperidin-1- | |
| | | | | yl)methyl]pyrrolidine-1- | |
| | | | | carboxylate | |

The compounds in the following table (i.e. Examples 033 to 034) were prepared in the same manner as in Example 028 except that the 3-ethyl-1-[5-(piperidin-4-yl)-1,2,4-oxadiazol-3-yl]-1H-indazole trifluoroacetate and (S)-(−)-1-tert-butoxycarbonyl-2-pyrrolidinecarbaldehyde were replaced with the corresponding starting compound and (R)-(+)-1-tert-butoxycarbonyl-2-pyrrolidinecarbaldehyde, respectively.

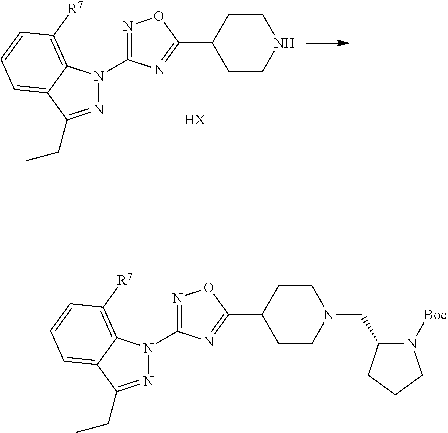

Wherein HX is hydrochloric acid or trifluoroacetic acid.

TABLE 18

| Ex. | R⁷ | Compound Name | LC-MS, m/z |
|---|---|---|---|
| 033 | H | tert-butyl (2R)-2-({4-[3-(3-ethyl-1H- | LC-MS, m/z; |
| | | indazol-1-yl)-1,2,4-oxadiazol-5- | 481 [M + H]+ |
| | | yl]piperidin-1-yl}methyl)pyrrolidine-1- | |
| | | carboxylate | |
| 034 | F | tert-butyl (2R)-2-({4-[3-(3-ethyl-7- | LC-MS, m/z; |
| | | fluoro-1H-indazol-1-yl)-1,2,4- | 499 [M + H]+ |
| | | oxadiazol-5-yl]piperidin-1- | |
| | | yl}methyl)pyrrolidine-1-carboxylate | |

### Example 035

### Preparation of tert-butyl (3S)-3-({4-[3-(3-ethyl-1H-indazol-1-yl)-1,2,4-oxadiazol-5-yl]piperidin-1-yl}methyl)pyrrolidine-1-carboxylate

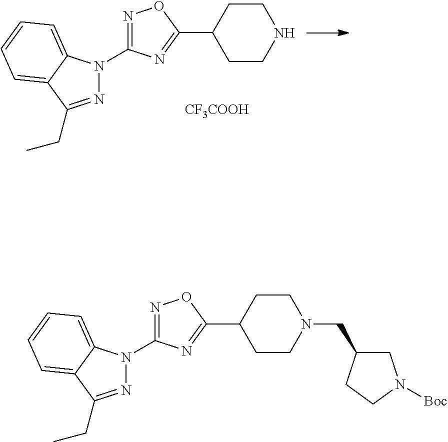 [SMILES: CCc1nn(-c2noc(C3CCNCC3)n2)c2ccccc12 ; "CF3COOH" ; CCc1nn(-c2noc(C3CCN(C[C@@H]4CCN(C(=O)OC(C)(C)C)C4)CC3)n2)c2ccccc12]

3-Ethyl-1-[5-(piperidin-4-yl)-1,2,4-oxadiazol-3-yl]-1H-indazole trifluoroacetate (100 mg) was suspended in N,N-dimethylformamide (3 ml). To the suspension were added tert-butyl (3R)-3-(iodomethyl)pyrrolidine-1-carboxylate (106 mg) and potassium carbonate (135 mg), and the mixture was refluxed overnight. The reaction solution was cooled to room temperature and water was added thereto. The mixture was extracted with ethyl acetate, the organic layer was washed with water and brine, dried over sodium sulfate and filtered, and the filtrate was concentrated under reduced pressure. The residue was purified by silica-gel chromatography (column; Hi-Flash™ Amino Column, developing solvent: hexane/ethyl acetate=1:1) to give the title compound (84 mg) as a white solid.

LC-MS, m/z; 481 [M+H]+

The compounds in the following table (i.e. Examples 036 to 038) were prepared in the same manner as in Example 035 except that the 3-ethyl-1-[5-(piperidin-4-yl)-1,2,4-oxadiazol-3-yl]-1H-indazole trifluoroacetate was replaced with the corresponding starting compound.

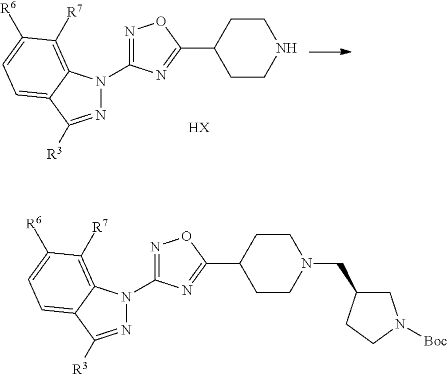

Wherein HX is hydrochloric acid or trifluoroacetic acid.

TABLE 19

| Ex. | R³ | R⁶ | R⁷ | Compound Name | LC-MS, m/z |
|---|---|---|---|---|---|
| 036 | Et | H | F | tert-butyl (3S)-3-({4-[3-(3- | LC-MS, m/z; |
| | | | | ethyl-7-fluoro-1H-indazol-1- | 499 [M + H]+ |
| | | | | yl)-1,2,4-oxadiazol-5- | |
| | | | | yl]piperidin-1- | |
| | | | | yl}methyl)pyrrolidine-1- | |
| | | | | carboxylate | |
| 037 | Et | F | H | tert-butyl (3S)-3-({4-[3-(3- | LC-MS, m/z; |
| | | | | ethyl-6-fluoro-1H-indazol-1- | 499 [M + H]+ |
| | | | | yl)-1,2,4-oxadiazol-5- | |
| | | | | yl]piperidin-1- | |
| | | | | yl}methyl)pyrrolidine-1- | |
| | | | | carboxylate | |
| 038 | ⁱPr | H | F | tert-butyl (3S)-3-[(4-{3-[7- | LCMS, m/z; |
| | | | | fluoro-3-(propan-2-yl)-1H- | 513 [M + H]+ |
| | | | | indazol-1-yl]-1,2,4-oxadiazol- | |
| | | | | 5-yl}piperidin-1- | |
| | | | | yl)methyl]pyrrolidine-1- | |
| | | | | carboxylate | |

The compounds in the following table (i.e. Examples 039 to 042) were prepared in the same manner as in Example 035 except that the 3-ethyl-1-[5-(piperidin-4-yl)-1,2,4-oxadiazol-3-yl]-1H-indazole trifluoroacetate and tert-butyl (3R)-3-(iodomethyl)pyrrolidine-1-carboxylate were replaced with the corresponding starting compound and tert-butyl (3S)-3-(iodomethyl)pyrrolidine-1-carboxylate, respectively.

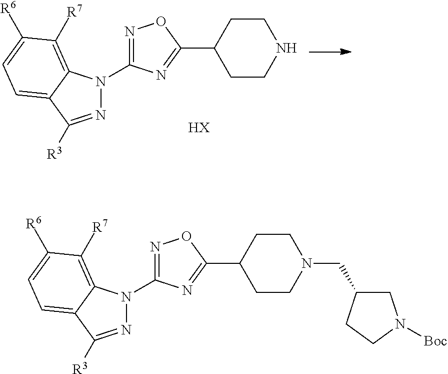

Wherein HX is hydrochloric acid or trifluoroacetic acid.

TABLE 20

| Ex. | R³ | R⁶ | R⁷ | Compound Name | LC-MS, m/z |
|---|---|---|---|---|---|
| 039 | Et | H | H | tert-butyl (3R)-3-({4-[3-(3- | LC-MS, m/z; |
| | | | | ethyl-1H-indazol-1-yl)-1,2,4- | 481 [M + H]+ |
| | | | | oxadiazol-5-yl]piperidin-1- | |
| | | | | yl}methyl)pyrrolidine-1- | |
| | | | | carboxylate | |
| 040 | Et | H | F | tert-butyl (3R)-3-({4-[3-(3- | LC-MS, m/z; |
| | | | | ethyl-7-fluoro-1H-indazol-1- | 499 [M + H]+ |
| | | | | yl)-1,2,4-oxadiazol-5- | |
| | | | | yl]piperidin-1- | |
| | | | | yl}methyl)pyrrolidine-1- | |
| | | | | carboxylate | |
| 041 | Et | F | H | tert-butyl (3R)-3-({4-[3-(3- | LC-MS, m/z; |
| | | | | ethyl-6-fluoro-1H-indazol-1- | 499 [M + H]+ |
| | | | | yl)-1,2,4-oxadiazol-5- | |
| | | | | yl]piperidin-1- | |
| | | | | yl}methyl)pyrrolidine-1- | |
| | | | | carboxylate | |
| 042 | ⁱPr | H | F | tert-butyl (3R)-3-[(4-{3-[7- | LCMS, m/z; |
| | | | | fluoro-3-(propan-2-yl)-1H- | 513 [M + H]+ |
| | | | | indazol-1-yl]-1,2,4-oxadiazol- | |
| | | | | 5-yl}piperidin-1- | |
| | | | | yl)methyl]pyrrolidine-1- | |
| | | | | carboxylate | |

The compounds in the following table (i.e. Examples 043 to 046) were prepared in the same manner as in Example 028 except that the 3-ethyl-1-[5-(piperidin-4-yl)-1,2,4-oxadiazol-3-yl]-1H-indazole trifluoroacetate and (S)-(−)-1-tert-butoxycarbonyl-2-pyrrolidinecarbaldehyde were replaced with the corresponding starting compound and tert-butyl 3-formylazetidine-1-carboxylate, respectively.

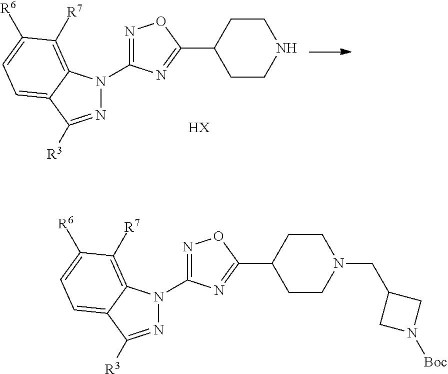

Wherein HX is hydrochloric acid or trifluoroacetic acid.

TABLE 21

| Ex. | R³ | R⁶ | R⁷ | Compound Name | LC-MS, m/z |
|---|---|---|---|---|---|
| 043 | Et | F | H | tert-butyl 3-({4-[3-(3-ethyl- | LC-MS, m/z; |
| | | | | 6-fluoro-1H-indazol-1-yl)- | 485 [M + H]+ |
| | | | | 1,2,4-oxadiazol-5- | |
| | | | | yl]piperidin-1- | |
| | | | | yl}methyl)azetidine-1- | |
| | | | | carboxylate | |
| 045 | Et | H | F | tert-butyl 3-({4-[3-(3-ethyl- | LC-MS, m/z; |
| | | | | 7-fluoro-1H-indazol-1-yl)- | 485 [M + H]+ |
| | | | | 1,2,4-oxadiazol-5- | |
| | | | | yl]piperidin-1- | |
| | | | | yl}methyl)azetidine-1- | |
| | | | | carboxylate | |
| 046 | ⁱPr | H | F | tert-butyl 3-[(4-{3-[7-fluoro- | LC-MS, m/z; |
| | | | | 3-(propan-2-yl)-1H-indazol-1- | 499 [M + H]+ |
| | | | | yl]-1,2,4-oxadiazol-5- | |
| | | | | yl}piperidin-1- | |
| | | | | yl)methyl]azetidine-1- | |
| | | | | carboxylate | |

The compounds in the following table (i.e. Reference Examples 047 to 051) were prepared in the same manner as in Example 028 except that the 3-ethyl-1-[5-(piperidin-4-yl)-1,2,4-oxadiazol-3-yl]-1H-indazole trifluoroacetate and (S)-(−)-1-tert-butoxycarbonyl-2-pyrrolidinecarbaldehyde were replaced with the corresponding starting compound and tert-butyl 4-oxopiperidine-1-carboxylate, respectively.

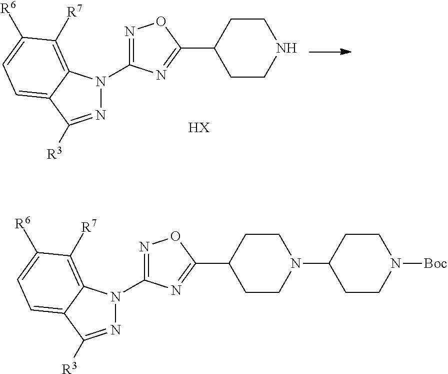

Wherein HX is hydrochloric acid or trifluoroacetic acid.

TABLE 22

| Ex. | R³ | R⁶ | R⁷ | Compound Name | LC-MS, m/z |
|---|---|---|---|---|---|
| 047 | Et | H | H | tert-butyl 4-[3-(3- | LC-MS, m/z; |
| | | | | ethyl-1H-indazol-1- | 481 [M + H]+ |
| | | | | yl)-1,2,4- | |
| | | | | oxadiazol-5-yl]- | |
| | | | | 1,4′-bipiperidine- | |
| | | | | 1′-carboxylate | |
| 048 | Et | F | H | tert-butyl 4-[3-(3- | LC-MS, m/z; |
| | | | | ethyl-6-fluoro-1H- | 499 [M + H]+ |
| | | | | indazol-1-yl)- | |
| | | | | 1,2,4-oxadiazol-5- | |
| | | | | yl]-1,4′- | |
| | | | | bipiperidine-1′- | |
| | | | | carboxylate | |
| 049 | Et | H | F | tert-butyl 4-[3-(3- | LC-MS, m/z; |
| | | | | ethyl-7-fluoro-1H- | 499 [M + H]+ |
| | | | | indazol-1-yl)- | |
| | | | | 1,2,4-oxadiazol-5- | |
| | | | | yl]-1,4′- | |
| | | | | bipiperidine-1′- | |
| | | | | carboxylate | |
| 050 | ⁱPr | H | F | tert-butyl 4-{3-[7- | LC-MS, m/z; |
| | | | | fluoro-3-(propan-2- | 513 [M + H]+ |
| | | | | yl)-1H-indazol-1- | |
| | | | | yl]-1,2,4- | |
| | | | | oxadiazol-5-yl}- | |
| | | | | 1,4′-bipiperidine- | |
| | | | | 1′-carboxylate | |
| 051 | Et | H | Cl | tert-butyl 4-[3-(7- | LC-MS, m/z; |
| | | | | chloro-3-ethyl-1H- | 515 [M + H]+ |
| | | | | indazol-1-yl)- | |
| | | | | 1,2,4-oxadiazol-5- | |
| | | | | yl]-1,4′- | |
| | | | | bipiperidine-1′- | |
| | | | | carboxylate | |

### Example 052

### Preparation of tert-butyl 4-{3-[3-(3-ethyl-1H-indazol-1-yl)-1,2,4-oxadiazol-5-yl]azetidin-1-yl}piperidine-1-carboxylate

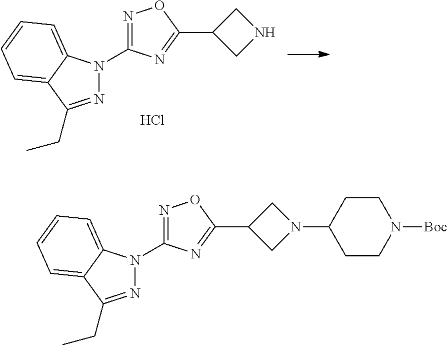 [SMILES: CCc1nn(-c2noc(C3CNC3)n2)c2ccccc12 ; Cl ; CCc1nn(-c2noc(C3CN(C4CCN(C(=O)OC(C)(C)C)CC4)C3)n2)c2ccccc12]

The title compound was prepared in the same manner as in Example 028 except that the 3-ethyl-1-[5-(piperidin-4-yl)-1,2,4-oxadiazol-3-yl]-1H-indazole trifluoroacetate and (S)-(−)-1-tert-butoxycarbonyl-2-pyrrolidinecarbaldehyde were replaced with 1-[5-(azetidin-3-yl)-1,2,4-oxadiazol-3-yl]-3-ethyl-1H-indazole hydrochloride and tert-butyl 4-oxopiperidine-1-carboxylate, respectively.

LC-MS, m/z; 396 [M+H-tBu]+

### Example 053

### Preparation of 7-fluoro-1-{5-[1-(piperidin-4-ylmethyl)piperidin-4-yl]-1,2,4-oxadiazol-3-yl}-3-(propan-2-yl)-1H-indazole dihydrochloride

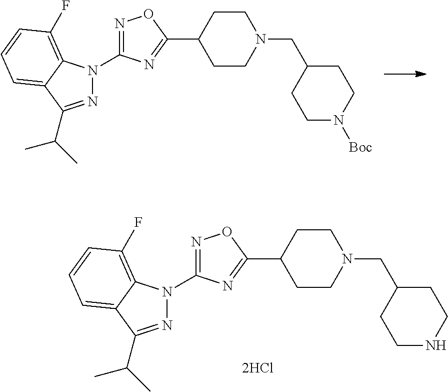 [SMILES: CC(C)c1nn(-c2noc(C3CCN(CC4CCN(C(=O)OC(C)(C)C)CC4)CC3)n2)c2c(F)cccc12 ; CC(C)c1nn(-c2noc(C3CCN(CC4CCNCC4)CC3)n2)c2c(F)cccc12]

To tert-butyl 4-[(4-{3-[7-fluoro-3-(propan-2-yl)-1H-indazol-1-yl]-1,2,4-oxadiazol-5-yl}piperidin-1-yl)methyl]piperidine-1-carboxylate (1.69 g) was added 4N HCl/dioxane (13.5 ml) at 0° C., and the mixture was reacted at room temperature for 3 hours. The reaction solution was concentrated under reduced pressure, toluene (5 ml) was added thereto and the mixture was concentrated under reduced pressure (×3). The residue was crystallized by adding ethyl acetate. Then, the resultant was concentrated under reduced pressure to give 7-fluoro-1-{5-[1-(piperidin-4-ylmethyl)piperidin-4-yl]-1,2,4-oxadiazol-3-yl}-3-(propan-2-yl)-1H-indazole dihydrochloride (1.59 g) as a colorless crystal.

LCMS, m/z; 427[M+H]+

### Example 054

### Preparation of 1-{5-[1-(azetidin-3-ylmethyl)piperidin-4-yl]-1,2,4-oxadiazol-3-yl}-7-fluoro-3-(propan-2-yl)-1H-indazole bis(trifluoroacetate)

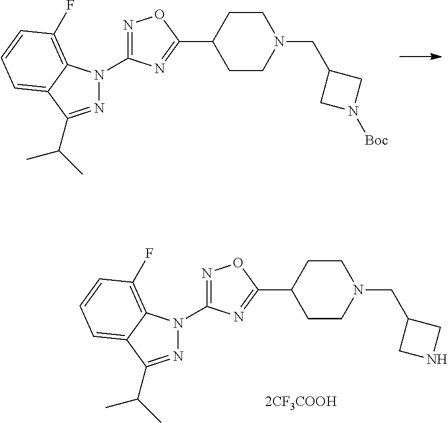 [SMILES: CC(C)c1nn(-c2noc(C3CCN(CC4CN(C(=O)OC(C)(C)C)C4)CC3)n2)c2c(F)cccc12 ; CC(C)c1nn(-c2noc(C3CCN(CC4CNC4)CC3)n2)c2c(F)cccc12]

Tert-butyl 3-[(4-{3-[7-fluoro-3-(propan-2-yl)-1H-indazol-1-yl]-1,2,4-oxadiazol-5-yl}piperidin-1-yl)methyl]azetidine-1-carboxylate (1.65 g) was dissolved in dichloromethane (4.00 ml). To the solution was added trifluoroacetic acid (4.00 ml), and the mixed solution was stirred at room temperature for 1 hour. The reaction solution was evaporated under reduced pressure, and the residue was crystallized from diethyl ether (20 ml). The resultant crystal was collected on a filter to give the title compound (1.96 g) as a white solid.

LC-MS, m/z; 399 [M+H]+

The compounds in the following table (i.e. Examples 055 to 080) were prepared in the same manner as in Example 053 or Example 054 except that the tert-butyl 4-[(4-{3-[7-fluoro-3-(propan-2-yl)-1H-indazol-1-yl]-1,2,4-oxadiazol-5-yl}piperidin-1-yl)methyl]piperidine-1-carboxylate of Example 053 or the tert-butyl 3-[(4-{3-[7-fluoro-3-(propan-2-yl)-1H-indazol-1-yl]-1,2,4-oxadiazol-5-yl}piperidin-1-yl)methyl]azetidine-1-carboxylate of Example 054 was replaced with the corresponding starting compound.

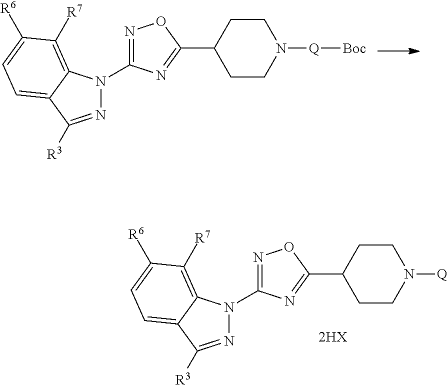

Wherein Q means each cyclic amino structure shown in the following table, HX means hydrochloric acid or trifluoroacetic acid, and the Boc group is attached to the nitrogen atom in the cyclic amine of Q.

TABLE 23

| | | | | | Compound | |
|---|---|---|---|---|---|---|
| Ex. | R³ | R⁶ | R⁷ | Q | Name | LC-MS, m/z |
| 055 | Et | H | H | 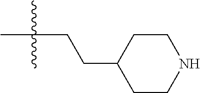 [SMILES: *CCC1CCNCC1] | 3-ethyl-1-(5-{1-[2- (piperidin-4- yl)ethyl]piperidin-4- yl}-1,2,4-oxadiazol- 3-yl)-1H-indazole bis(trifluoroacetate) | LC-MS, m/z; 409 [M + H]+ |
| 056 | Et | H | H | 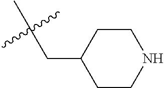 [SMILES: *CC1CCNCC1] | 3-ethyl-1-{5-[1- (piperidin-4- ylmethyl)piperidin-4- yl]-1,2,4-oxadiazol- 3-yl}-1H-indazole bis(trifluoroacetate) | LC-MS, m/z; 395 [M + H]+ |
| 057 | Et | F | H | | 3-ethyl-6-fluoro-1- | LC-MS, m/z; |
| | | | | | {5-[1-(piperidin-4- | 413 [M + H]+ |
| | | | | | ylmethyl)piperidin-4- | |
| | | | | | yl]-1,2,4-oxadiazol- | |
| | | | | | 3-yl}-1H-indazole | |
| | | | | | dihydrochloride | |
| 058 | Et | H | F | 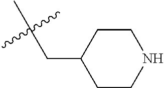 [SMILES: *CC1CCNCC1] | 3-ethyl-7-fluoro-1- {5-[1-(piperidin-4- ylmethyl)piperidin-4- yl]-1,2,4-oxadiazol- 3-yl}-1H-indazole dihydrochloride | LC-MS, m/z; 413 [M + H]+ |
| 059 | Et | H | H | 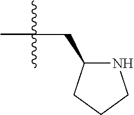 [SMILES: *C[C@@H]1CCCN1] | 3-ethyl-1-(5-{1- [(2S)-pyrrolidin-2- ylmethyl]piperidin-4- yl}-1,2,4-oxadiazol- 3-yl)-1H-indazole bis(trifluoroacetate) | LC-MS, m/z; 381 [M + H]+ |
| 060 | Me | F | H | | 6-fluoro-3-methyl-1- | LC-MS, m/z; |
| | | | | | (5-{1-[(2S)- | 385 [M + H]+ |
| | | | | | pyrrolidin-2- | |
| | | | | | ylmethyl]piperidin-4- | |
| | | | | | yl}-1,2,4-oxadiazol- | |
| | | | | | 3-yl)-1H-indazole | |
| | | | | | bis(trifluoroacetate) | |
| 061 | Et | F | H | | 3-ethyl-6-fluoro-1- | LC-MS, m/z; |
| | | | | | (5-{1-[(2S)- | 399 [M + H]+ |
| | | | | | pyrrolidin-2- | |
| | | | | | ylmethyl]piperidin-4- | |
| | | | | | yl}-1,2,4-oxadiazol- | |
| | | | | | 3-yl)-1H-indazole | |
| | | | | | bis(trifluoroacetate) | |
| 062 | Et | H | F | | 3-ethyl-7-fluoro-1- | LC-MS, m/z; |
| | | | | | (5-{1-[(2S)- | 399 [M + H]+ |
| | | | | | pyrrolidin-2- | |
| | | | | | ylmethyl]piperidin-4- | |
| | | | | | yl}-1,2,4-oxadiazol- | |
| | | | | | 3-yl)-1H-indazole | |
| | | | | | dihydrochloride | |
| 063 | ⁱPr | H | F | | 7-fluoro-3-(propan-2- | LC-MS, m/z; |
| | | | | | yl)-1-(5-{1-[(2S)- | 413 [M + H]+ |
| | | | | | pyrrolidin-2- | |
| | | | | | ylmethyl]piperidin-4- | |
| | | | | | yl}-1,2,4-oxadiazol- | |
| | | | | | 3-yl)-1H-indazole | |
| | | | | | dihydrochloride | |
| 064 | Et | H | H | 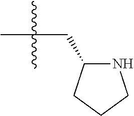 [SMILES: *C[C@H]1CCCN1] | 3-ethyl-1-(5-{1- [(2R)-pyrrolidin-2- ylmethyl]piperidin-4- yl}-1,2,4-oxadiazol- 3-yl)-1H-indazole bis(trifluoroacetate) | LC-MS, m/z; 381 [M + H]+ |
| 065 | Et | H | F | | 3-ethyl-7-fluoro-1- | LC-MS, m/z; |
| | | | | | (5-{1-[(2R)- | 399 [M + H]+ |
| | | | | | pyrrolidin-2- | |
| | | | | | ylmethyl]piperidin-4- | |
| | | | | | yl}-1,2,4-oxadiazol- | |
| | | | | | 3-yl)-1H-indazole | |
| | | | | | dihydrochloride | |
| 066 | Et | H | H | 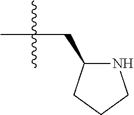 [SMILES: *C[C@@H]1CCCN1] | 3-ethyl-1-(5-{1- [(3R)-pyrrolidin-3- ylmethyl]piperidin-4- yl}-1,2,4-oxadiazol- 3-yl)-1H-indazole bis(trifluoroacetate) | LC-MS, m/z; 381 [M + H]+ |
| 067 | Et | H | F | | 3-ethyl-7-fluoro-1- | LC-MS, m/z; |
| | | | | | (5-{1-[(3R)- | 399 [M + H]+ |
| | | | | | pyrrolidin-3- | |
| | | | | | ylmethyl]piperidin-4- | |
| | | | | | yl}-1,2,4-oxadiazol- | |
| | | | | | 3-yl)-1H-indazole | |
| | | | | | dihydrochloride | |
| 068 | Et | F | H | | 3-ethyl-6-fluoro-1- | LC-MS, m/z; |
| | | | | | (5-{1-[(3R)- | 399 [M + H]+ |
| | | | | | pyrrolidin-3- | |
| | | | | | ylmethyl]piperidin-4- | |
| | | | | | yl}-1,2,4-oxadiazol- | |
| | | | | | 3-yl)-1H-indazole | |
| | | | | | dihydrochloride | |
| 069 | ⁱPr | H | F | | 7-fluoro-3-(propan-2- | LC-MS, m/z; |
| | | | | | yl)-1-(5-{1-[(3R)- | 413 [M + H]+ |
| | | | | | pyrrolidin-3- | |
| | | | | | ylmethyl]piperidin-4- | |
| | | | | | yl}-1,2,4-oxadiazol- | |
| | | | | | 3-yl)-1H-indazole | |
| | | | | | dihydrochloride | |
| 070 | Et | H | H | 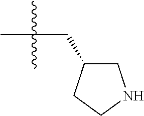 [SMILES: *C[C@H]1CCNC1] | 3-ethyl-1-(5-{1- [(3S)-pyrrolidin-3- ylmethyl]piperidin-4- yl}-1,2,4-oxadiazol- 3-yl)-1H-indazole bis(trifluoroacetate) | LC-MS, m/z; 381 [M + H]+ |
| 071 | Et | H | F | | 3-ethyl-7-fluoro-1- | LC-MS, m/z; |
| | | | | | (5-{1-[(3S)- | 399 [M + H]+ |
| | | | | | pyrrolidin-3- | |
| | | | | | ylmethyl]piperidin-4- | |
| | | | | | yl}-1,2,4-oxadiazol- | |
| | | | | | 3-yl)-1H-indazole | |
| | | | | | dihydrochloride | |
| 072 | Et | F | H | | 3-ethyl-6-fluoro-1- | LC-MS, m/z; |
| | | | | | (5-{1-[(3S)- | 399 [M + H]+ |
| | | | | | pyrrolidin-3- | |
| | | | | | ylmethyl]piperidin-4- | |
| | | | | | yl}-1,2,4-oxadiazol- | |
| | | | | | 3-yl)-1H-indazole | |
| | | | | | dihydrochloride | |
| 073 | ⁱPr | H | F | | 7-fluoro-3-(propan-2- | LC-MS, m/z; |
| | | | | | yl)-1-(5-{1-[(3S)- | 413 [M + H]+ |
| | | | | | pyrrolidin-3- | |
| | | | | | ylmethyl]piperidin-4- | |
| | | | | | yl}-1,2,4-oxadiazol- | |
| | | | | | 3-yl)-1H-indazole | |
| | | | | | dihydrochloride | |
| 074 | Et | F | H | 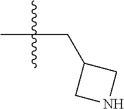 [SMILES: *CC1CNC1] | 1-{5-[1-(azetidin-3- ylmethyl)piperidin-4- yl]-1,2,4-oxadiazol- 3-yl}-3-ethyl-6- fluoro-1H-indazole bis(trifluoroacetate) | LC-MS, m/z; 385 [M + H]+ |
| 075 | Et | H | F | | 1-{5-[1-(azetidin-3- | LC-MS, m/z; |
| | | | | | ylmethyl)piperidin-4- | 385 [M + H]+ |
| | | | | | yl]-1,2,4-oxadiazol- | |
| | | | | | 3-yl}-3-ethyl-7- | |
| | | | | | fluoro-1H-indazole | |
| | | | | | bis(trifluoroacetate) | |
| 076 | Et | H | H | 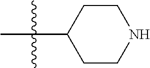 [SMILES: *C1CCNCC1] | 4-[3-(3-ethyl-1H- indazol-1-yl)-1,2,4- oxadiazol-5-yl]-1,4′- bipiperidine bis(trifluoroacetate) | LC-MS, m/z; 381 [M + H]+ |
| 077 | Et | F | H | | 4-[3-(3-ethyl-6- | LC-MS, m/z; |
| | | | | | fluoro-1H-indazol-1- | 399 [M + H]+ |
| | | | | | yl)-1,2,4-oxadiazol- | |
| | | | | | 5-yl]-1,4′- | |
| | | | | | bipiperidine | |
| | | | | | dihydrochloride | |
| 078 | Et | H | F | | 4-[3-(3-ethyl-7- | LC-MS, m/z; |
| | | | | | fluoro-1H-indazol-1- | 399 [M + H]+ |
| | | | | | yl)-1,2,4-oxadiazol- | |
| | | | | | 5-yl]-1,4′- | |
| | | | | | bipiperidine | |
| | | | | | bis(trifluoroacetate) | |
| 079 | ⁱPr | H | F | | 4-{3-[7-fluoro-3- | LC-MS, m/z; |
| | | | | | (propan-2-yl)-1H- | 413 [M + H]+ |
| | | | | | indazol-1-yl]-1,2,4- | |
| | | | | | oxadiazol-5-yl}-1,4′- | |
| | | | | | bipiperidine | |
| | | | | | dihydrochloride | |
| 080 | Et | H | Cl | | 4-[3-(7-chloro-3- | LC-MS, m/z; |
| | | | | | ethyl-1H-indazol-1- | 415 [M + H]+ |
| | | | | | yl)-1,2,4-oxadiazol- | |
| | | | | | 5-yl]-1,4′- | |
| | | | | | bipiperidine | |
| | | | | | dihydrochloride | |

### Example 081

### Preparation of 3-ethyl-1-{5-[1-(piperidin-4-yl)azetidin-3-yl]-1,2,4-oxadiazol-3-yl}-1H-indazole bis(trifluoroacetate)

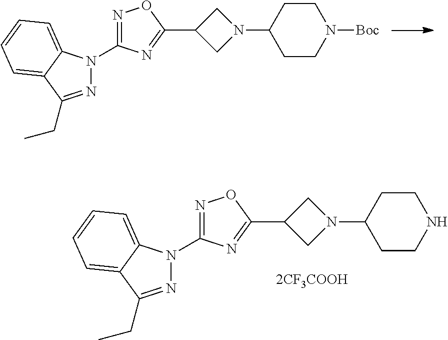 [SMILES: CCc1nn(-c2noc(C3CN(C4CCN(C(=O)OC(C)(C)C)CC4)C3)n2)c2ccccc12 ; CCc1nn(-c2noc(C3CN(C4CCNCC4)C3)n2)c2ccccc12]

The title compound was prepared in the same manner as in Example 054 except that the tert-butyl 3-[(4-{3-[7-fluoro-3-(propan-2-yl)-1H-indazol-1-yl]-1,2,4-oxadiazol-5-yl}piperidin-1-yl)methyl]azetidine-1-carboxylate was replaced with tert-butyl 4-{3-[3-(3-ethyl-1H-indazol-1-yl)-1,2,4-oxadiazol-5-yl]azetidin-1-yl}piperidine-1-carboxylate.

LC-MS, m/z; 353 [M+H]+

### Example 082

### Preparation of 3-{4-[3-(3-methyl-1H-indazol-1-yl)-1,2,4-oxadiazol-5-yl]piperidin-1-yl}propan-1-amine bis(trifluoroacetate)

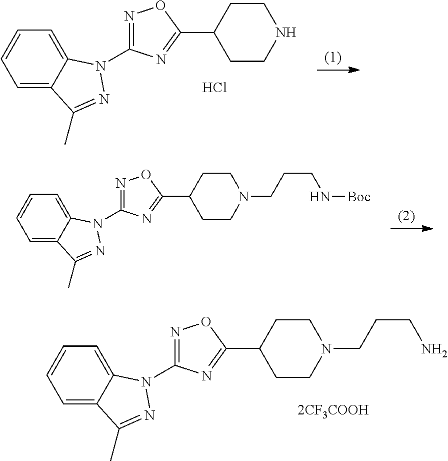 [SMILES: Cc1nn(-c2noc(C3CCNCC3)n2)c2ccccc12 ; Cl ; Cc1nn(-c2noc(C3CCN(CCCNC(=O)OC(C)(C)C)CC3)n2)c2ccccc12 ; Cc1nn(-c2noc(C3CCN(CCCN)CC3)n2)c2ccccc12]

(1) Tert-butyl(3-{4-[3-(3-methyl-1H-indazol-1-yl)-1,2,4-oxadiazol-5-yl]piperidin-1-yl}propyl)carbamate was prepared in the same manner as in Example 023 except that the 3-ethyl-1-[5-(piperidin-4-yl)-1,2,4-oxadiazol-3-yl]-1H-indazole trifluoroacetate and tert-butyl 4-(2-iodoethyl)piperidine-1-carboxylate were replaced with 3-methyl-1-[5-(piperidin-4-yl)-1,2,4-oxadiazol-3-yl]-1H-indazole hydrochloride and tert-butyl(3-bromopropyl)carbamate, respectively.

LC-MS, m/z; 441 [M+H]+.

(2) The title compound was prepared in the same manner as in Example 054 except that the tert-butyl 3-[(4-{3-[7-fluoro-3-(propan-2-yl)-1H-indazol-1-yl]-1,2,4-oxadiazol-5-yl}piperidin-1-yl)methyl]azetidine-1-carboxylate was replaced with the above compound.

LC-MS, m/z; 341 [M+H]+

The following compounds in the table (i.e. Examples 083 to 084) were prepared in the same manner as in Example 082 (or replacing the trifluoroacetic acid with 4 N HCl/dioxane) except that the 3-methyl-1-[5-(piperidin-4-yl)-1,2,4-oxadiazol-3-yl]-1H-indazole hydrochloride and tert-butyl(3-bromopropyl)carbamate were replaced with the corresponding starting compound and tert-butyl 2-bromoethylcarbamate, respectively.

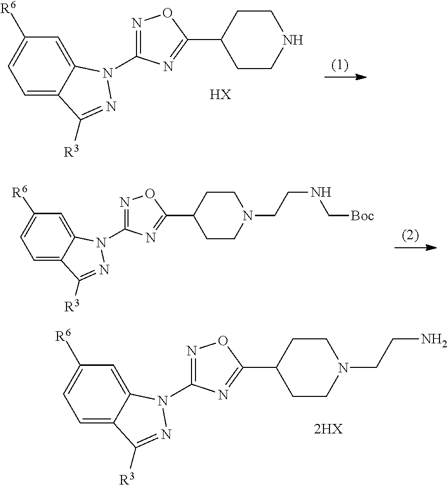

Wherein HX is hydrochloric acid or trifluoroacetic acid.

TABLE 24

| Ex. | R³ | R⁶ | Compound Name | LC-MS, m/z |
|---|---|---|---|---|
| 083 | Me | H | 2-{4-[3-(3-methyl-1H-indazol-1- | LC-MS, m/z; |
| | | | yl)-1,2,4-oxadiazol-5- | 327 [M + H]+ |
| | | | yl]piperidin-1-yl}ethanamine | |
| | | | bis(trifluoroacetate) | |
| 084 | Et | F | 2-{4-[3-(3-ethyl-6-fluoro-1H- | LC-MS, m/z; |
| | | | indazol-1-yl)-1,2,4-oxadiazol-5- | 359 [M + H]+ |
| | | | yl]piperidin-1-yl}ethanamine | |
| | | | dihydrochloride | |

### Example 085

### Preparation of 1-{5-[1-(3-methoxypropyl)piperidin-4-yl]-1,2,4-oxadiazol-3-yl}-3-(propan-2-yl)-1H-indazole

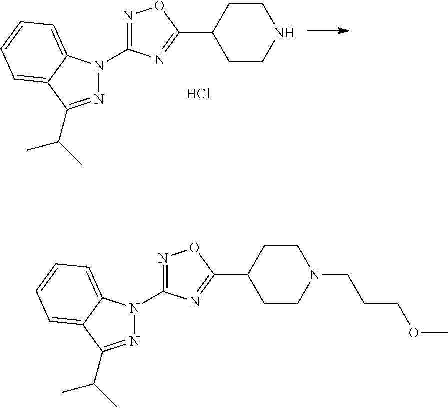 [SMILES: CC(C)c1nn(-c2noc(C3CCNCC3)n2)c2ccccc12 ; Cl ; COCCCN1CCC(c2nc(-n3nc(C(C)C)c4ccccc43)no2)CC1]

1-[5-(Piperidin-4-yl)-1,2,4-oxadiazol-3-yl]-3-(propan-2-yl)-1H-indazole hydrochloride (174 mg) was suspended in DMF (3 ml). To the suspension were added 1-bromo-3-methoxy propane (92 mg), potassium carbonate (138 mg) and sodium iodide (15 mg), and the mixture was stirred at 60° C. for 1.5 hours and then cooled to room temperature. To the reaction mixture was added water, and the mixture was extracted with chloroform. The organic layer was dried over sodium sulfate and filtered, and the filtrate was concentrated under reduced pressure. The residue was purified by silica-gel chromatography (column; Hi-Flash™ Column, developing solvent: hexane/ethyl acetate=1/1 then chloroform/methanol=9/1) to give the title compound (145 mg) as a colorless solid.

¹H-NMR (CDCl₃) δ: 1.52 (6H, d, J=7.0 Hz), 1.74-1.85 (2H, m), 2.03-2.22 (6H, m), 2.42-2.49 (2H, m), 2.96-3.10 (3H, m), 3.35 (3H, s), 3.44 (2H, t, J=6.4 Hz), 3.47-3.57 (1H, m), 7.28-7.34 (1H, m), 7.52-7.58 (1H, m), 7.80-7.85 (1H, m), 8.27-8.32 (1H, m).

LC-MS, m/z; 384 [M+H]+.

The compounds in the following table (i.e. Examples 086 to 095) were prepared in the same manner as in Example 085 except that the 1-[5-(piperidin-4-yl)-1,2,4-oxadiazol-3-yl]-3-(propan-2-yl)-1H-indazole hydrochloride was replaced with the corresponding starting compound. In order to obtain each of the trifluoroacetates in the following table, the crude product was isolated/purified by reverse phase HPLC.

TABLE 25

| | Chemical | | |
|---|---|---|---|
| Ex. | structure | Compound Name | ¹H-NMR/LC-MS, m/z |
| 086 | 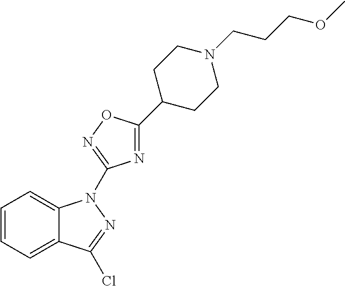 [SMILES: COCCCN1CCC(c2nc(-n3nc(Cl)c4ccccc43)no2)CC1] | 3-chloro-1-{5-[1- (3-methoxy- propyl)piper- idin-4-yl]-1,2,4- oxadiazol-3-yl}- 1H-indazole | ¹H-NMR (CDCl₃) δ: 1.74- 1.85 (2H, m), 2.02-2.24 (6H, m), 2.42-2.50 (2H, m), 2.95-3.11 (3H, m), 3.35 (3H, s), 3.44 (2H, t, J = 6.4 Hz), 7.39- 7.45 (1H, m), 7.62-7.68 (1H, m), 7.75-7.80 (1H, m), 8.28-8.32 (1H, m). LC-MS, m/z; 376 [M + H] + |
| 087 | 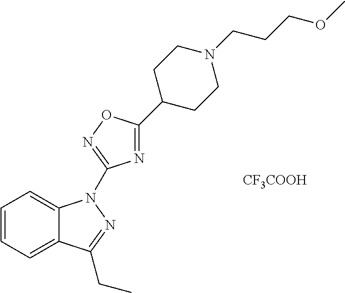 [SMILES: CCc1nn(-c2noc(C3CCN(CCCOC)CC3)n2)c2ccccc12 ; "CF3COOH"] | 3-ethyl-1-{5-[1- (3-methoxy- propyl)piper- idin-4-yl]-1,2,4- oxadiazol-3-yl}- 1H-indazole trifluoroacetate | LC-MS, m/z; 370 [M + H] + |
| 088 | 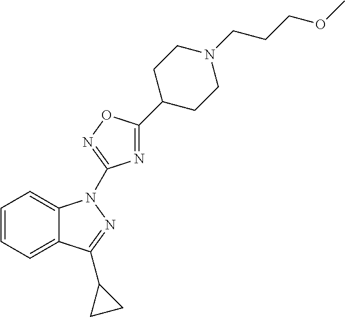 [SMILES: COCCCN1CCC(c2nc(-n3nc(C4CC4)c4ccccc43)no2)CC1] | 3-cyclopropyl-1- {5-[1-(3-methoxy- propyl)piper- idin-4-yl]-1,2,4- oxadiazol-3-yl}- 1H-indazole trifluoroacetate | LC-MS, m/z; 382 [M + H] + |
| 089 | 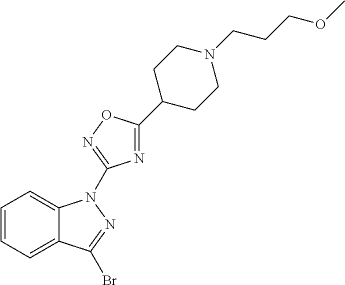 [SMILES: COCCCN1CCC(c2nc(-n3nc(Br)c4ccccc43)no2)CC1] | 3-bromo-1-{5-[1- (3-methoxy- propyl)piper- idin-4-yl]-1,2,4- oxadiazol-3-yl}- 1H-indazole | LC-MS, m/z; 420 [M + H] + |
| 090 | 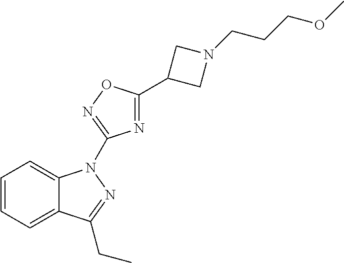 [SMILES: CCc1nn(-c2noc(C3CN(CCCOC)C3)n2)c2ccccc12] | 3-ethyl-1-{5-[1- (3-methoxy- propyl)azeti- din-3-yl]-1,2,4- oxadiazol-3-yl}- 1H-indazole | LC-MS, m/z; 342 [M + H] + |
| 091 | 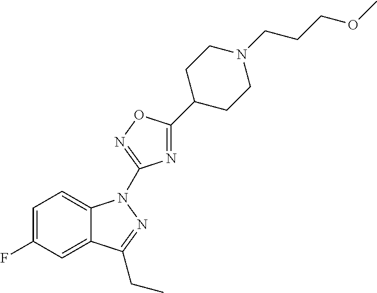 [SMILES: CCc1nn(-c2noc(C3CCN(CCCOC)CC3)n2)c2ccc(F)cc12] | 3-ethyl-5-fluoro- 1-{5-[1-(3-methoxy- propyl)piper- idin-4-yl]-1,2,4- oxadiazol-3-yl}- 1H-indazole | ¹H-NMR (CDCl₃) δ: 1.43 (3H, t, J = 8.0 Hz), 1.75-1.84 (2H, m), 1.99- 2.23 (6H, m), 2.46 (2H, t, J = 7.4 Hz), 2.96- 3.10 (5H, m), 3.34 (3H, s), 3.44 (2H, t, J = 6.3 Hz), 7.31 (1H, td, J = 8.9, 2.4 Hz), 7.38 (1H, dd, J = 8.0, 2.4 Hz), 8.24 (1H, dd, J = 9.1,4.3 Hz). LC-MS, m/z; 388 [M + H] + |
| 092 | 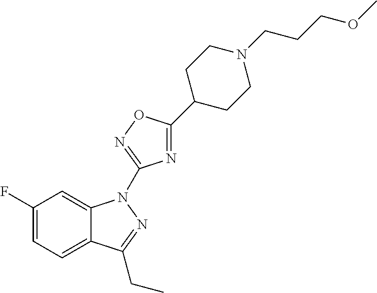 [SMILES: CCc1nn(-c2noc(C3CCN(CCCOC)CC3)n2)c2cc(F)ccc12] | 3-ethyl-6-fluoro- 1-{5-[1-(3- methoxypropyl)piper- idin-4-yl]-1,2,4- oxadiazol-3-yl}- 1H-indazole | ¹H-NMR (CDCl₃) δ: 1.44 (3H, t, J = 7.7 Hz), 1.70-1.85 (2H, m), 1.98- 2.22 (6H, m), 2.40-2.49 (2H, m), 2.93-3.10 (5H, m), 3.35 (3H, s), 3.44 (2H, t, J = 6.3 Hz), 7.08 (1H, td, J = 8.8, 2.2 Hz), 7.69 (1H, dd, J = 8.7, 5.0 Hz), 7.98 (1H, dd, J = 9.3, 1.7 Hz). LC-MS, m/z; 388 [M + H] + |
| 093 | 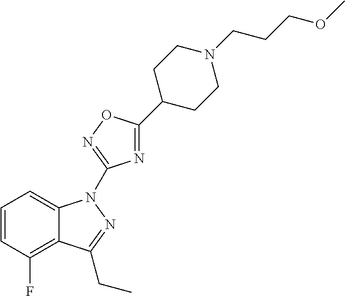 [SMILES: CCc1nn(-c2noc(C3CCN(CCCOC)CC3)n2)c2cccc(F)c12] | 3-ethyl-4-fluoro- 1-{5-[1-(3- methoxypropyl)piper- idin-4-yl]-1,2,4- oxadiazol-3-yl}- 1H-indazole | ¹H-NMR (CDCl₃) δ: 1.43 (3H, t, J = 7.6 Hz), 1.73-1.84 (2H, m), 1.98- 2.22 (6H, m), 2.46 (2H, t, J = 7.4 Hz), 2.95- 3.09 (3H, m), 3.15 (2H, q, J = 7.6 Hz), 3.34 (3H, s), 3.44 (2H, t, J = 6.5 Hz), 6.95 (1H, dd, J = 10.0, 7.8 Hz), 7.49 (1H, td, J = 8.2, 5.0 Hz), 8.06 (1H, d, J = 8.5 Hz). LC-MS, m/z; 388 [M + H] + |
| 094 | 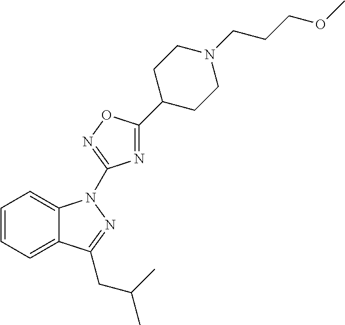 [SMILES: COCCCN1CCC(c2nc(-n3nc(CC(C)C)c4ccccc43)no2)CC1] | 1-{5-[1-(3- methoxypropyl)piper- idin-4-yl]-1,2,4- oxadiazol-3-yl}-3- (2-methylpropyl)- 1H-indazole | ¹H-NMR (CDCl₃) δ: 1.01 (6H, d, J = 6.6 Hz), 1.70-1.85 (2H, m), 2.00- 2.33 (7H, m), 2.46 (2H, t, J = 7.6 Hz), 2.90- 3.11 (5H, m), 3.35 (3H, s), 3.44 (2H, t, J = 6.5 Hz), 7.29-7.36 (1H, m), 7.51-7.60 (1H, m), 7.74 (1H, d, J = 8.0 Hz), 8.29 (1H, d, J = 8.5 Hz). LC-MS, m/z; 398 [M + H] + |
| 095 | 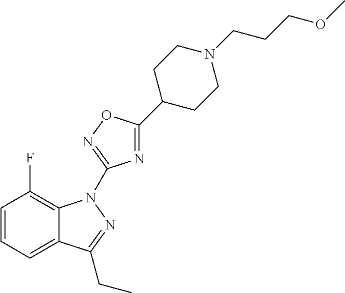 [SMILES: CCc1nn(-c2noc(C3CCN(CCCOC)CC3)n2)c2c(F)cccc12] | 3-ethyl-7-fluoro- 1-{5-[1-(3- methoxypropyl)piper- idin-4-yl]-1,2,4- oxadiazol-3-yl}- 1H-indazole | ¹H-NMR (CDCl₃) δ: 1.44 (3H, t, J = 7.6 Hz), 1.71-1.85 (2H, m), 1.99- 2.23 (6H, m), 2.45 (2H, t, J = 7.6 Hz), 2.93- 3.13 (5H, m), 3.34 (3H, s), 3.44 (2H, t, J = 6.5 Hz), 7.19-7.30 (2H, m), 7.50-7.56 (1H, m). LC-MS, m/z; 388 [M + H] + |

### Example 096

### Preparation of 3-(cyclohex-1-en-1-yl)-1-{5-[1-(3-methoxypropyl)piperidin-4-yl]-1,2,4-oxadiazol-3-yl}-1H-indazole

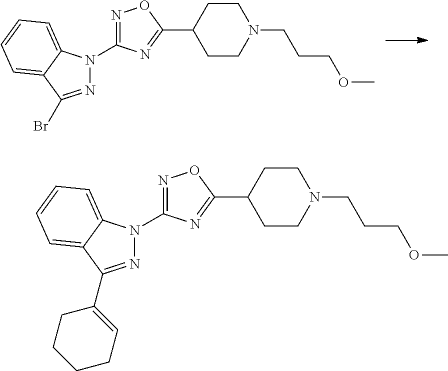 [SMILES: COCCCN1CCC(c2nc(-n3nc(Br)c4ccccc43)no2)CC1 ; COCCCN1CCC(c2nc(-n3nc(C4=CCCCC4)c4ccccc43)no2)CC1]

3-Bromo-1-{5-[1-(3-methoxypropyl)piperidin-4-yl]-1,2,4-oxadiazol-3-yl}-1H-indazole (80 mg) was suspended in 1,4-dioxane (4 ml) and water (0.5 ml). To the suspension were added 2-(1-cyclohexenyl)-4,4,5,5-tetramethyl-1,3,2-dioxaborolane (52 mg), tetrakistriphenylphosphinepalladium (11 mg) and potassium carbonate (79 mg), and the mixture was refluxed overnight. Then, the mixture was cooled to room temperature, and water was added thereto. The mixture was extracted with ethyl acetate. The organic layer was washed with water and brine, dried over sodium sulfate, filtered, and concentrated under reduced pressure. The residue was purified by silica-gel chromatography (column; Hi-Flash™ Amino Column, developing solvent: hexane/ethyl acetate) to give the title compound (19 mg) as a white solid.

LC-MS, m/z; 422 [M+H]+

### Example 097

### Preparation of 3-ethyl-1-[5-(1-ethylpiperidin-4-yl)-1,2,4-oxadiazol-3-yl]-6-fluoro-1H-indazole hydrochloride

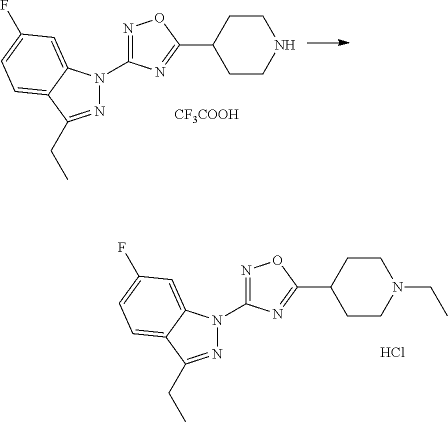 [SMILES: CCc1nn(-c2noc(C3CCNCC3)n2)c2cc(F)ccc12 ; "CF3COOH" ; CCc1nn(-c2noc(C3CCN(CC)CC3)n2)c2cc(F)ccc12 ; Cl]

3-Ethyl-6-fluoro-1-[5-(piperidin-4-yl)-1,2,4-oxadiazol-3-yl]-1H-indazole trifluoroacetate (100 mg) was suspended in N,N-dimethylformamide (2 ml). To the suspension were added ethyl iodide (45 mg) and potassium carbonate (133 mg), and the mixture was refluxed overnight. The reaction solution was cooled to room temperature and water was added thereto. The mixture was extracted with ethyl acetate. The organic layer was washed with water and brine, dried over sodium sulfate and filtered, and the filtrate was concentrated under reduced pressure. The residue was purified by silica-gel chromatography (column; Hi-Flash™ Amino Column, developing solvent: hexane/ethyl acetate). The resultant compound was dissolved in methylene chloride and treated with 1N HCl/diethyl ether to give the title compound (35 mg) as a white solid.

¹H-NMR (DMSO-d₆) δ: 1.20-1.30 (3H, m), 1.34 (3H, t, J=7.4 Hz), 2.10-2.49 (5H, m), 2.98-3.16 (6H, m), 3.60 (2H, d, J=11.7 Hz), 7.26-7.32 (1H, m), 7.87-7.95 (1H, m), 7.99-8.05 (1H, m), 10.17 (1H, s).

LC-MS, m/z; 344 [M+H]+

The compounds in the following table (i.e. Examples 098 to 0133) were prepared in the same manner as in Example 097 except that the 3-ethyl-6-fluoro-1-[5-(piperidin-4-yl)-1,2,4-oxadiazol-3-yl]-1H-indazole trifluoroacetate and ethyl iodide were replaced with the corresponding starting compound and R—X which means an alkylating agent, respectively. In order to obtain each of the trifluoroacetates in the following table, the residue was isolated/purified by reverse phase HPLC, and each free form of the compounds in the following table was obtained by omitting the conversion step into hydrochloride in Example 097.

TABLE 26

| | | | | ¹H-NMR/ |
|---|---|---|---|---|
| | | | Compound | LC-MS, |
| Ex. | R—X | Chemical structure | Name | m/z |
| 098 | 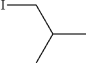 | 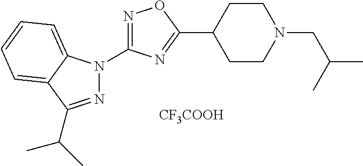 [SMILES: CC(C)CN1CCC(c2nc(-n3nc(C(C)C)c4ccccc43)no2)CC1 ; "CF3COOH"] | 1-{5-[1-(2- methylpropyl)pi- peridin-4-yl]- 1,2,4- oxadiazol-3- yl}-3-(propan- 2-yl)-1H- indazole trifluoroacetate | LC-MS, m/z; 368 [M + H] + |
| 099 | 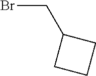 | 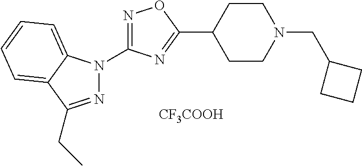 [SMILES: CCc1nn(-c2noc(C3CCN(CC4CCC4)CC3)n2)c2ccccc12 ; "CF3COOH"] | 1-{5-[1- (cyclobutylmeth- yl)piperidin-4- yl]-1,2,4- oxadiazol-3- yl}-3-ethyl-1H- indazole trifluoroacetate | LC-MS, m/z; 366 [M + H] + |
| 100 | 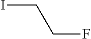 | 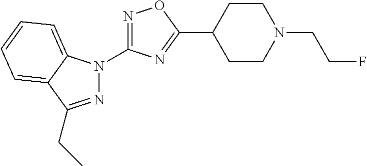 [SMILES: CCc1nn(-c2noc(C3CCN(CCF)CC3)n2)c2ccccc12] | 3-ethyl-1-{5- [1-(2- fluoroethyl)pi- peridin-4-yl]- 1,2,4- oxadiazol-3- yl}-1H-indazole | LC-MS, m/z; 344 [M + H] + |
| 101 | 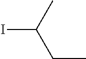 | 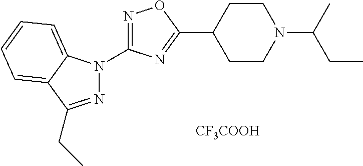 [SMILES: CCc1nn(-c2noc(C3CCN(C(C)CC)CC3)n2)c2ccccc12 ; "CF3COOH"] | 1-{5-[1-(butan- 2-yl)piperidin- 4-yl]-1,2,4- oxadiazol-3- yl}-3-ethyl-1H- indazole trifluoroacetate | LC-MS, m/z; 354 [M + H] + |
| 102 | 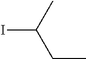 | 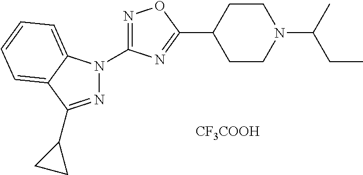 [SMILES: CCC(C)N1CCC(c2nc(-n3nc(C4CC4)c4ccccc43)no2)CC1 ; "CF3COOH"] | 1-{5-[1-(butan- 2-yl)piperidin- 4-yl]-1,2,4- oxadiazol-3- yl}-3- cyclopropyl-1H- indazole trifluoroacetate | LC-MS, m/z; 366 [M + H] + |
| 103 | 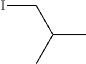 | 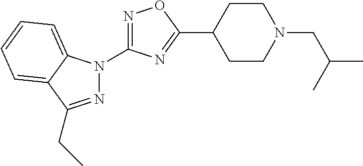 [SMILES: CCc1nn(-c2noc(C3CCN(CC(C)C)CC3)n2)c2ccccc12] | 3-ethyl-1-(5- [1-(2- methylpropyl)pi- peridin-4-yl]- 1,2,4- oxadiazol-3- yl}-1H-indazole | LC-MS, m/z; 354 [M + H] + |
| 104 | 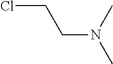 | 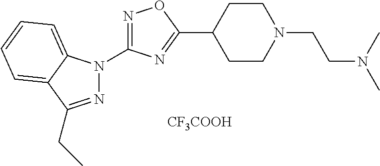 [SMILES: CCc1nn(-c2noc(C3CCN(CCN(C)C)CC3)n2)c2ccccc12 ; "CF3COOH"] | 2-{4-[3-(3- ethyl-1H- indazol-1-yl)- 1,2,4- oxadiazol-5- yl]piperidin-1- yl}-N,N- dimethylethanamine trifluoroacetate | LC-MS, m/z; 369 [M + H] + |
| 105 | 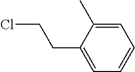 | 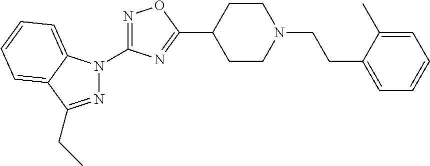 [SMILES: CCc1nn(-c2noc(C3CCN(CCc4ccccc4C)CC3)n2)c2ccccc12] | 3-ethyl-1-(5- {1-[2-(2- methylphenyl)eth- yl]piperidin- 4-yl}-1,2,4- oxadiazol-3- yl)-1H-indazole | LC-MS, m/z; 416 [M + H] + |
| 106 | 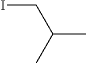 | 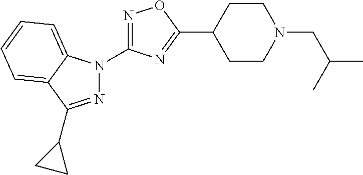 [SMILES: CC(C)CN1CCC(c2nc(-n3nc(C4CC4)c4ccccc43)no2)CC1] | 3-cyclopropyl- 1-{5-[1-(2- methylpropyl)pi- peridin-4-yl]- 1,2,4- oxadiazol-3- yl}-1H-indazole | LC-MS, m/z; 366 [M + H] + |
| 107 | 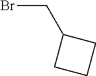 | 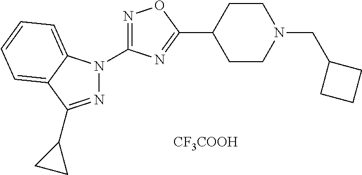 [SMILES: c1ccc2c(c1)c(C1CC1)nn2-c1noc(C2CCN(CC3CCC3)CC2)n1 ; "CF3COOH"] | 1-{5-[1- (cyclobutylmeth- yl)piperidin-4- yl]-1,2,4- oxadiazol-3- yl}-3- cyclopropyl-1H- indazole trifluoroacetate | LC-MS, m/z; 378 [M + H] + |
| 108 | 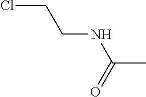 | 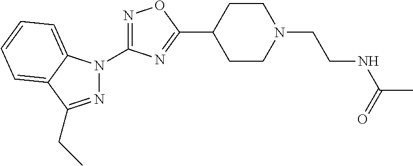 [SMILES: CCc1nn(-c2noc(C3CCN(CCNC(C)=O)CC3)n2)c2ccccc12] | N-(2-{4-[3-(3- ethyl-1H- indazol-1-yl)- 1,2,4- oxadiazol-5- yl]piperidin-1- yl}ethyl)acetamide | LC-MS, m/z; 383 [M + H] + |
| 109 | 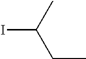 | 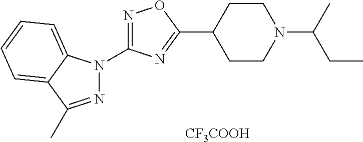 [SMILES: CCC(C)N1CCC(c2nc(-n3nc(C)c4ccccc43)no2)CC1 ; "CF3COOH"] | 1-{5-[1-(butan- 2-yl)piperidin- 4-yl]-1,2,4- oxadiazol-3- yl}-3-methyl- 1H-indazole trifluoroacetate | LC-MS, m/z; 340 [M + H] + |
| 110 | 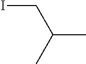 | 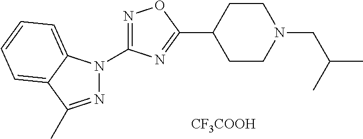 [SMILES: Cc1nn(-c2noc(C3CCN(CC(C)C)CC3)n2)c2ccccc12 ; "CF3COOH"] | 3-methyl-1-{5- [1-(2- methylpropyl)pi- peridin-4-yl]- 1,2,4- oxadiazol-3- yl}-1H-indazole trifluoroacetate | LC-MS, m/z; 340 [M + H] + |
| 111 | 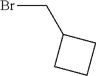 | 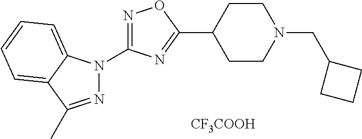 [SMILES: Cc1nn(-c2noc(C3CCN(CC4CCC4)CC3)n2)c2ccccc12 ; "CF3COOH"] | 1-{5-[1- (cyclobutylmeth- yl)piperidin-4- yl]-1,2,4- oxadiazol-3- yl}-3-methyl- 1H-indazole trifluoroacetate | LC-MS, m/z; 352 [M + H] + |
| 112 | 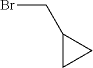 | 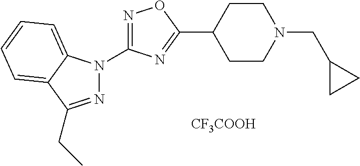 [SMILES: CCc1nn(-c2noc(C3CCN(CC4CC4)CC3)n2)c2ccccc12 ; "CF3COOH"] | 1-{5-[1- (cyclopropylmeth- yl)piperidin- 4-yl]-1,2,4- oxadiazol-3- yl}-3-ethyl-1H- indazole trifluoroacetate | LC-MS, m/z; 352 [M + H] + |
| 113 | 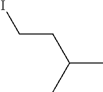 | 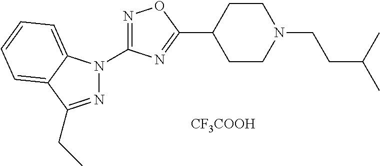 [SMILES: CCc1nn(-c2noc(C3CCN(CCC(C)C)CC3)n2)c2ccccc12 ; "CF3COOH"] | 3-ethyl-1-{5- [1-(3- methylbutyl)piper- idin-4-yl]- 1,2,4- oxadiazol-3- yl}-1H-indazole trifluoroacetate | LC-MS, m/z; 368 [M + H] + |
| 114 | 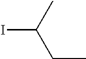 | 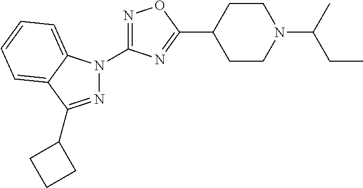 [SMILES: CCC(C)N1CCC(c2nc(-n3nc(C4CCC4)c4ccccc43)no2)CC1] | 1-{5-[1-(butan- 2-yl)piperidin- 4-yl]-1,2,4- oxadiazol-3- yl}-3- cyclobutyl-1H- indazole trifluoroacetate | LC-MS, m/z; 380 [M + H] + |
| 115 | 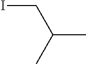 | 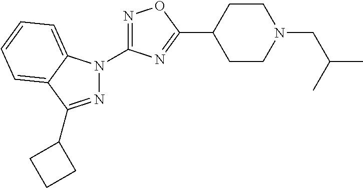 [SMILES: CC(C)CN1CCC(c2nc(-n3nc(C4CCC4)c4ccccc43)no2)CC1] | 3-cyclobutyl-1- {5-[1-{2- methylpropyl)pi- peridin-4-yl]- 1,2,4- oxadiazol-3- yl}-1H-indazole trifluoroacetate | LC-MS, m/z; 380 [M + H] + |
| 116 | 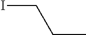 | 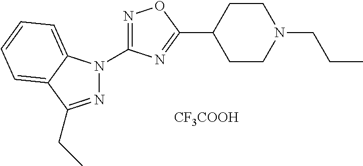 [SMILES: CCCN1CCC(c2nc(-n3nc(CC)c4ccccc43)no2)CC1 ; "CF3COOH"] | 3-ethyl-1-[5-(1- propylpiperidin- 4-yl)-1,2,4- oxadiazol-3- yl]-1H-indazole trifluoroacetate | LC-MS, m/z; 340 [M + H] + |
| 117 | 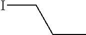 | 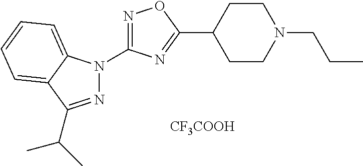 [SMILES: CCCN1CCC(c2nc(-n3nc(C(C)C)c4ccccc43)no2)CC1 ; "CF3COOH"] | 3-(propan-2- yl)-1-[5-(1- propylpiperidin- 4-yl)-1,2,4- oxadiazol-3- yl]-1H-indazole trifluoroacetate | LC-MS, m/z; 354 [M + H] + |
| 118 | 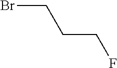 | 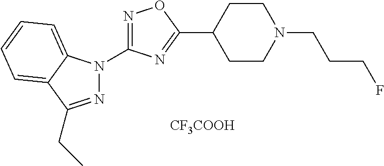 [SMILES: CCc1nn(-c2noc(C3CCN(CCCF)CC3)n2)c2ccccc12 ; "CF3COOH"] | 3-ethyl-1-{5- [1-(3- fluoropropyl)pi- peridin-4-yl]- 1,2,4- oxadiazol-3- yl}-1H-indazole trifluoroacetate | LC-MS, m/z; 358 [M + H] + |
| 119 | 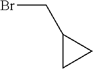 | 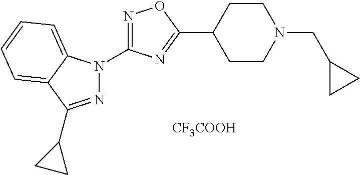 [SMILES: c1ccc2c(c1)c(C1CC1)nn2-c1noc(C2CCN(CC3CC3)CC2)n1 ; "CF3COOH"] | 3-cyclopropyl- 1-{5-[1- (cyclopropylmeth- yl)piperidin- 4-yl]-1,2,4- oxadiazol-3- yl}-1H-indazole trifluoroacetate | LC-MS, m/z; 364 [M + H] + |
| 120 | 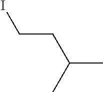 | 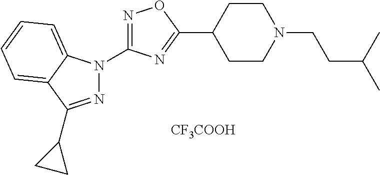 [SMILES: CC(C)CCN1CCC(c2nc(-n3nc(C4CC4)c4ccccc43)no2)CC1 ; "CF3COOH"] | 3-cyclopropyl- 1-{5-[1-(3- methylbutyl)piper- idin-4-yl]- 1,2,4- oxadiazol-3- yl}-1H-indazole trifluoroacetate | LC-MS, m/z; 380 [M + H] + |
| 121 | 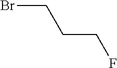 | 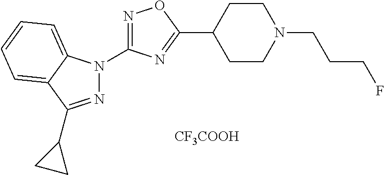 [SMILES: FCCCN1CCC(c2nc(-n3nc(C4CC4)c4ccccc43)no2)CC1 ; "CF3COOH"] | 3-cyclopropyl- 1-{5-[1-(3- fluoropropyl)pi- peridin-4-yl]- 1,2,4- oxadiazol-3- yl}-1H-indazole trifluoroacetate | LC-MS, m/z; 370 [M + H] + |
| 122 | 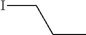 | 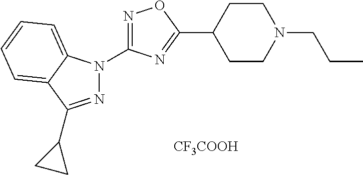 [SMILES: CCCN1CCC(c2nc(-n3nc(C4CC4)c4ccccc43)no2)CC1 ; "CF3COOH"] | 3-cyclopropyl- 1-[5-(1- propylpiperidin- 4-yl)-1,2,4- oxadiazol-3- yl]-1H-indazole trifluoroacetate | LC-MS, m/z; 352 [M + H] + |
| 123 | 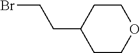 | 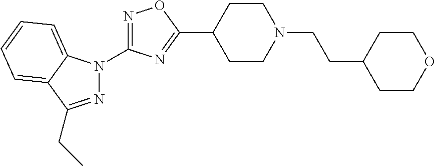 [SMILES: CCc1nn(-c2noc(C3CCN(CCC4CCOCC4)CC3)n2)c2ccccc12] | 3-ethyl-1-(5- {1-[2- (tetrahydro-2H- pyran-4- yl)ethyl]piper- idin-4-yl}- 1,2,4- oxadiazol-3- yl)-1H-indazole | LC-MS, m/z; 410 [M + H] + |
| 124 | 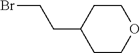 | 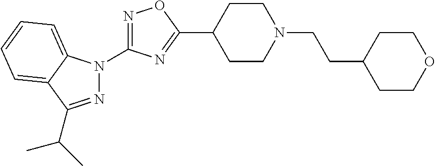 [SMILES: CC(C)c1nn(-c2noc(C3CCN(CCC4CCOCC4)CC3)n2)c2ccccc12] | 3-(propan-2- yl)-1-(5-{1-[2- (tetrahydro-2H- pyran-4- yl)ethyl]piper- idin-4-yl}- 1,2,4- oxadiazol-3- yl)-1H-indazole | LC-MS, m/z; 424 [M + H] + |
| 125 | 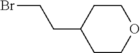 | 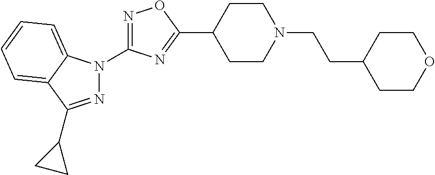 [SMILES: c1ccc2c(c1)c(C1CC1)nn2-c1noc(C2CCN(CCC3CCOCC3)CC2)n1] | 3-cyclopropyl- 1-(5-{1-[2- (tetrahydro-2H- pyran-4- yl)ethyl]piper- idin-4-yl}- 1,2,4- oxadiazol-3- yl)-1H-indazole | LC-MS, m/z; 422 [M + H] + |
| 126 | 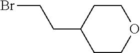 | 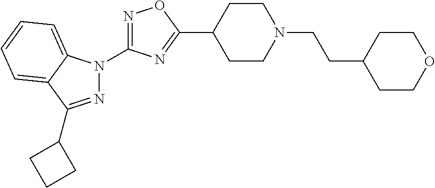 [SMILES: c1ccc2c(c1)c(C1CCC1)nn2-c1noc(C2CCN(CCC3CCOCC3)CC2)n1] | 3-cyclobutyl-1- (5-{1-[2- (tetrahydro-2H- pyran-4- yl)ethyl]piper- idin-4-yl}- 1,2,4- oxadiazol-3- yl)-1H-indazole | LC-MS, m/z; 436 [M + H] + |
| 127 | 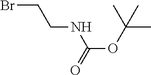 | 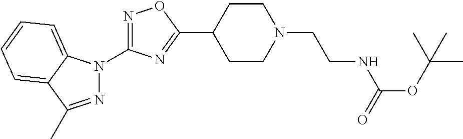 [SMILES: Cc1nn(-c2noc(C3CCN(CCNC(=O)OC(C)(C)C)CC3)n2)c2ccccc12] | tert-butyl (2- {4-[3-(3- methyl-1H- indazol-1-yl)- 1,2,4- oxadiazol-5- yl]piperidin-1- yl}ethyl)carbamate | ¹H-NMR (DMSO-d₆) δ: 1.37 (9H, s), 1.83 (2H, q, J = 10.7 Hz), 2.05-2.17 (4H, m), 2.34 (2H, t, J = 7.0 Hz), 2.60 (3H, s), 2.84- 2.91 (2H, m), 3.01- 3.15 (3H, m), 6.66 (1H, t, J = 5.0 Hz), 7.39 = (1H, t, J 7.6 Hz), 7.64 (1H, t, J = 7.7 Hz), 7.91 (1H, d, J = 8.0 Hz), 8.20 (1H, d, J = 8.5 Hz). LC-MS, m/z; 427 [M + H] + |
| 128 | 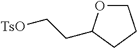 [SMILES: Cc1ccc(S(=O)(=O)OCCC2CCCO2)cc1] | 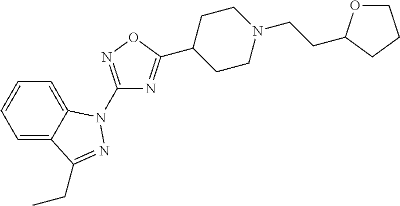 [SMILES: CCc1nn(-c2noc(C3CCN(CCC4CCCO4)CC3)n2)c2ccccc12] | 3-ethyl-1-(5- {1-[2- (tetrahydrofuran-2- yl)ethyl]piper- idin-4-yl}- 1,2,4- oxadiazol-3- yl)-1H-indazole | ¹H-NMR (DMSO-d₆) δ: 1.33- 1.43 (4H, m), 1.62 (2H, q, J = 7.0 Hz), 1.74-1.98 (5.0H, m), 2.12 (3H, d, J = 11.7 Hz), 2.91-2.96 (1H, m), 3.03 (2H, q, J = 7.6 Hz), 3.12-3.20 (1H, m), 3.31-3.34 (2H, m), 3.43-3.50 (2H, m), 3.53-3.59 (1H, m), 3.69-3.76 (2H, m), 7.38 (1H, m), 7.64 (1H, m), 7.94 (1H, d, J = 8.0 Hz), 8.20 (1H, d, J = 8.5 Hz). LC-MS, m/z; 396 [M + H] + |
| 129 | 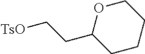 [SMILES: Cc1ccc(S(=O)(=O)OCCC2CCCCO2)cc1] | 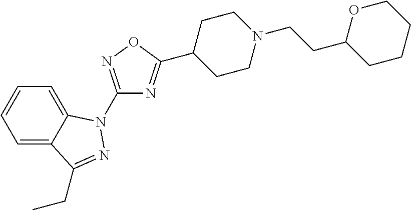 [SMILES: CCc1nn(-c2noc(C3CCN(CCC4CCCCO4)CC3)n2)c2ccccc12] | 3-ethyl-1-(5- {1-[2- (tetrahydro-2H- pyran-2- yl)ethyl]piper- idin-4-yl}- 1,2,4- oxadiazol-3- yl)-1H-indazole | LC-MS, m/z; 410 [M + H] + |
| 130 | 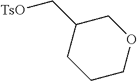 [SMILES: Cc1ccc(S(=O)(=O)OCC2CCCOC2)cc1] | 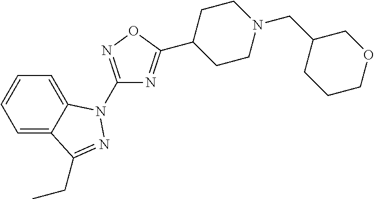 [SMILES: CCc1nn(-c2noc(C3CCN(CC4CCCOC4)CC3)n2)c2ccccc12] | 3-ethyl-1-{5- [1-(tetrahydro- 2H-pyran-3- ylmethyl)piper- idin-4-yl]- 1,2,4- oxadiazol-3- yl}-1H-indazole | ¹H-NMR (DMSO-d₆) δ: 1.10- 1.21 (1H, m), 1.35 (3H, t, J = 7.6 Hz), 1.40- 1.58 (2H, m), 1.71-1.94 (4H, m), 2.07-2.38 (4H, m), 2.78-3.20 (6H, m), 3.25-3.31 (1H, m), 3.69-3.82 (4H, m), 7.36-7.40 (1H, m), 7.61-7.66 (1H, m), 7.93 (1H, d, J = 8.0 Hz), 8.19 (1H, d, J = 8.5 Hz). LC-MS, m/z; 396 [M + H] + |
| 131 | 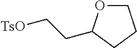 [SMILES: Cc1ccc(S(=O)(=O)OCCC2CCCO2)cc1] | 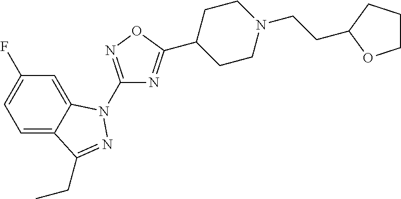 [SMILES: CCc1nn(-c2noc(C3CCN(CCC4CCCO4)CC3)n2)c2cc(F)ccc12] | 3-ethyl-6- fluoro-1-(5-{1-[2- (tetrahydrofuran-2- yl)ethyl]piper- idin-4-yl}- 1,2,4- oxadiazol-3- yl)-1H-indazole | ¹H-NMR (CDCl₃) δ: 1.36-1.57 (4H, m), 1.64-2.25 (11H, m), 2.39-2.60 (2H, m), 2.95-3.16 (5H, m), 3.67-3.94 (3H, m), 7.08 (1H, td, J = 8.8, 2.2 Hz), 7.69 (1H, dd, J = 8.7, 5.0 Hz), 7.98 (1H, dd, J = 9.5, 2.2 Hz). LC-MS, m/z; 414 [M + H] + |
| 132 | 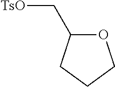 [SMILES: Cc1ccc(S(=O)(=O)OCC2CCCO2)cc1] | 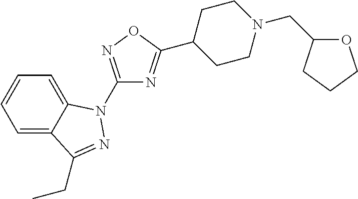 [SMILES: CCc1nn(-c2noc(C3CCN(CC4CCCO4)CC3)n2)c2ccccc12] | 3-ethyl-1-{5- [1-(tetrahydrofuran- 2-ylmethyl)piper- idin-4-yl]- 1,2,4- oxadiazol-3- yl}-1H-indazole | LC-MS, m/z; 382 [M + H] + |
| 133 | 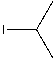 | 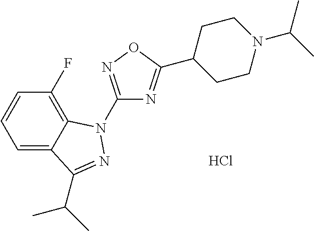 [SMILES: CC(C)c1nn(-c2noc(C3CCN(C(C)C)CC3)n2)c2c(F)cccc12 ; Cl] | 7-fluoro-3- (propan-2-yl)- 1-{5-[1- (propan-2- yl)piperidin-4- yl]-1,2,4- oxadiazol-3- yl}-1H-indazole hydrochloride | ¹H-NMR (DMSO-d₆) δ: 1.31 (6H, d, J = 6.6Hz), 1.41 (6H, d, J = 7.1 Hz), 2.24-2.43 (4H, m), 3.08-3.21 (2H, m), 3.42-3.59 (5H, m), 7.35-7.43 (1H, m), 7.44-7.52 (1H, m), 7.81-7.89 (1H, m), 10.60- 10.85 (1H, m). LC-MS, m/z; 372 [M + H] + |

### Example 134

### Preparation of 3-ethyl-6-fluoro-1-{5-[1-(tetrahydro-2H-pyran-4-ylmethyl)piperidin-4-yl]-1,2,4-oxadiazol-3-yl}-1H-indazole

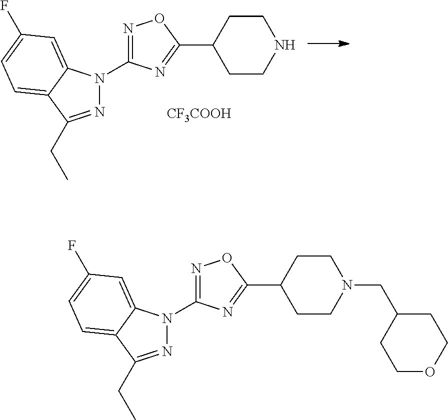 [SMILES: CCc1nn(-c2noc(C3CCNCC3)n2)c2cc(F)ccc12 ; "CF3COOH" ; CCc1nn(-c2noc(C3CCN(CC4CCOCC4)CC3)n2)c2cc(F)ccc12]

3-Ethyl-6-fluoro-1-[5-(piperidin-4-yl)-1,2,4-oxadiazol-3-yl]-1H-indazole trifluoroacetate (150 mg) was dissolved in dichloromethane (3 ml), and to the solution was added tetrahydropyrane-4-carboaldehyde (60 mg) and triacetoxysodium borohydride (222 mg). The mixed solution was stirred at room temperature for 3 hours. To the reaction solution was added saturated sodium bicarbonate aqueous solution (10 ml). The mixture was extracted with ethyl acetate (20 ml), and the organic layer was again washed with water (10 ml×2). The organic layer was dried over sodium sulfate and filtered, and the filtrate was concentrated under reduced pressure. The residue was purified by silica-gel chromatography (column; Hi-Flash™ Amino Column, developing solvent: hexane/ethyl acetate=2:1) to give the title compound (84 mg) as a white solid.

¹H-NMR (CDCl₃) δ: 1.27 (2H, ddd, J=24.9, 11.9, 4.2 Hz), 1.44 (3H, t, J=8.0 Hz), 1.63-1.83 (3H, m), 1.99-2.18 (6H, m), 2.22 (2H, d, J=7.1 Hz), 2.89-3.11 (5H, m), 3.40 (2H, t, J=10.9 Hz), 3.98 (2H, dd, J=11.3, 3.5 Hz), 7.08 (1H, td, J=8.8, 2.3 Hz), 7.70 (1H, dd, J=8.7, 5.0 Hz), 7.98 (1H, dd, J=9.4, 2.1 Hz).

LC-MS, m/z; 414 [M+H]⁺.

The compounds in the following table (i.e. Examples 135 to 159) were prepared in the same manner as in Example 134 except that the 3-ethyl-6-fluoro-1-[5-(piperidin-4-yl)-1,2,4-oxadiazol-3-yl]-1H-indazole trifluoroacetate and tetrahydropyrane-4-carboaldehyde were replaced with the corresponding starting compound and aldehyde or ketone, respectively. Each of the hydrochloride compounds in the following table was obtained by dissolving the resultant compound in methylene chloride and treating with 1N HCl/diethyl ether solution.

TABLE 27

| | Chemical | | ¹H-NMR/ |
|---|---|---|---|
| Ex. | structure | Compound Name | LC-MS, m/z |
| 135¹⁾ | 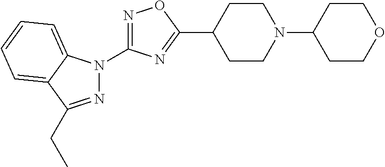 [SMILES: CCc1nn(-c2noc(C3CCN(C4CCOCC4)CC3)n2)c2ccccc12] | 3-ethyl-1-{5-[1- (tetrahydro-2H- pyran-4- yl)piperidin-4-yl]- 1,2,4-oxadiazol-3- yl}-1H-indazole | LC-MS, m/z; 382 [M + H] + |
| 136 | 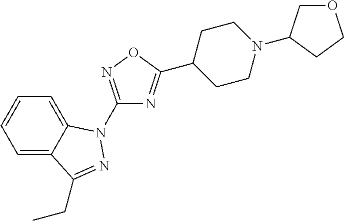 [SMILES: CCc1nn(-c2noc(C3CCN(C4CCOC4)CC3)n2)c2ccccc12] | 3-ethyl-1-{5-[1- (tetrahydrofuran-3- yl)piperidin-4-yl]- 1,2,4-oxadiazol-3- yl}-1H-indazole | LC-MS, m/z; 368 [M + H] + |
| 137 | 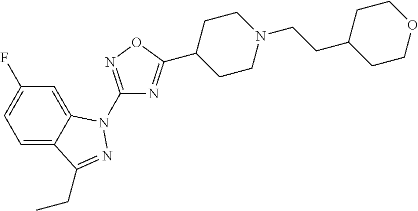 [SMILES: CCc1nn(-c2noc(C3CCN(CCC4CCOCC4)CC3)n2)c2cc(F)ccc12] | 3-ethyl-6-fluoro-1- (5-{1-[2- (tetrahydro-2H- pyran-4- yl)ethyl]piperidin- 4-yl}-1,2,4- oxadiazol-3-yl)-1H- indazole | ¹H-NMR (CDCl₃) δ: 1.25-1.68 (10H, m), 2.01-2.25 (6H, m), 2.41 (2H, t, J = 7.7 Hz), 2.93-3.14 (5H, m), 3.34- 3.46 (2H, m), 3.96 (2H, dd, J = 11.1, 4.0 Hz), 7.08 (1H, td, J = 8.8, 2.2 Hz), 7.70 (1H, dd, J = 8.8, 5.1 Hz), 7.98 (1H, dd, J = 9.3, 2.2 Hz). LC-MS, m/z; 428 [M + H] + |
| 138 | 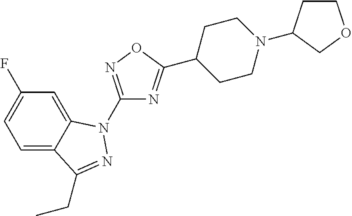 [SMILES: CCc1nn(-c2noc(C3CCN(C4CCOC4)CC3)n2)c2cc(F)ccc12] | 3-ethyl-6-fluoro-1- {5-[1- (tetrahydrofuran-3- yl)piperidin-4-yl]- 1,2,4-oxadiazol-3- yl}-1H-indazole | ¹H-NMR (CDCl₃) δ: 1.44 (3H, t, J = 7.6 Hz), 1.83- 1.95 (1H, m), 2.01-2.36 (7H, m), 2.81-2.91 (1H, m), 2.98- 3.15 (5H, m), 3.67 (1H, dd, J = 8.7, 7.0 Hz), 3.81 (1H, dd, J = 15.9, 8.3 Hz), 3.88-4.02 (2H, m), 7.08 (1H, td, J = 8.9, 2.2 Hz), 7.70 (1H, dd, J = 8.8, 5.1 Hz), 7.97 (1H, dd, J = 9.3, 2.2 Hz). LC-MS, m/z; 386 [M + H] + |
| 139 | 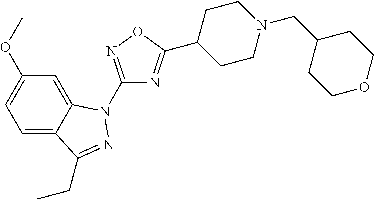 [SMILES: CCc1nn(-c2noc(C3CCN(CC4CCOCC4)CC3)n2)c2cc(OC)ccc12] | 3-ethyl-6-methoxy- 1-{5-[1- (tetrahydro-2H- pyran-4- ylmethyl)piperidin- 4-yl]-1,2,4- oxadiazol-3-yl}-1H- indazole | ¹H-NMR (CDCl₃) δ: 1.27 (2H, ddd, J = 24.9, 12.0, 4.4 Hz), 1.42 (3H, t, J = 7.7 Hz), 1.62-1.82 (3H, m), 1.98- 2.25 (8H, m), 2.89-2.98 (2H, m), 3.04 (3H, q, J = 7.6 Hz), 3.34-3.45 (2H, m), 3.91-4.02 (5H, m), 6.94 (1H, dd, J = 8.8, 2.2 Hz), 7.60 (1H, d, J = 8.8 Hz), 7.71 (1H, d, J = 2.0 Hz). LC-MS, m/z; 426 [M + H] + |
| 140 | 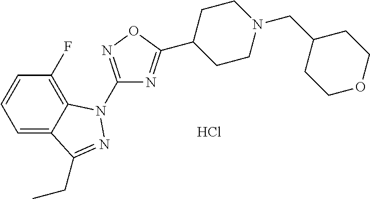 [SMILES: CCc1nn(-c2noc(C3CCN(CC4CCOCC4)CC3)n2)c2c(F)cccc12 ; Cl] | 3-ethyl-7-fluoro-1- {5-[1-{tetrahydro- 2H-pyran-4- ylmethyl)piperidin- 4-yl]-1,2,4- oxadiazol-3-yl}-1H- indazole hydrochloride | ¹H-NMR (DMSO-d₆) δ: 1.15-1.32 (2H, m), 1.36 (3H, t, J = 7.4 Hz), 1.71-1.83 (2H, m), 2.01- 2.20 (1H, m), 2.26-2.54 (4H, m), 2.93-3.19 (6H, m), 3.25- 3.55 (3H, m), 3.59-3.74 (2H, m), 3.79-3.91 (2H, m), 7.35- 7.53 (2H, m), 7.76-7.84 (1H; m), 10.40-10.65 (1H, m). LC-MS, m/z; 414 [M + H] + |
| 141 | 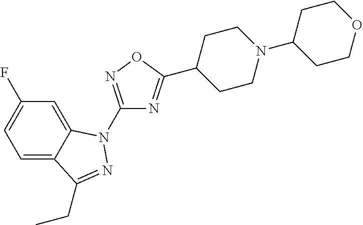 [SMILES: CCc1nn(-c2noc(C3CCN(C4CCOCC4)CC3)n2)c2cc(F)ccc12] | 3-ethyl-6-fluoro-1- {5-[1-(tetrahydro- 2H-pyran-4- yl)piperidin-4-yl]- 1,2,4-oxadiazol-3- yl}-1H-indazole | LC-MS, m/z; 400 [M + H] + |
| 142 | 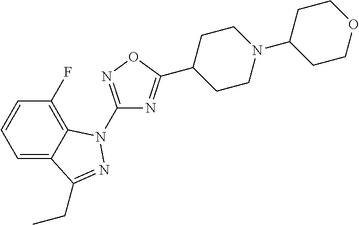 [SMILES: CCc1nn(-c2noc(C3CCN(C4CCOCC4)CC3)n2)c2c(F)cccc12] | 3-ethyl-7-fluoro-1- {5-[1-(tetrahydro- 2H-pyran-4- yl)piperidin-4-yl]- 1,2,4-oxadiazol-3- yl}-1H-indazole | LC-MS, m/z; 400 [M + H] + |
| 143 | 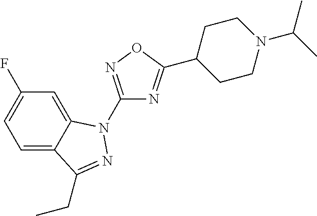 [SMILES: CCc1nn(-c2noc(C3CCN(C(C)C)CC3)n2)c2cc(F)ccc12] | 3-ethyl-6-fluoro-1- {5-[1-(propan-2- yl)piperidin-4-yl]- 1,2,4-oxadiazol-3- yl}-1H-indazole | ¹H-NMR (DMSO-d₆) δ: 0.97 (6H, d, J = 6.6 Hz), 1.34 (3H, t, J = 7.5 Hz), 1.72- 1.86 (2H, m), 2.05-2.15 (2H, m), 2.29 (2H, t, J = 10.5 Hz), 2.68-2.87 (3H, m), 2.97-3.13 (3H, m), 7.22- 7.30 (1H, m), 7.85-7.90 (1H, m), 7.95-8.02 (1H, m). LC-MS, m/z; 358 [M + H] + |
| 144 | 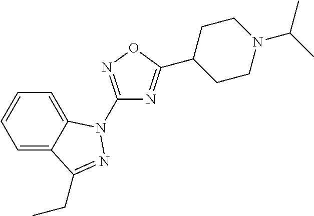 [SMILES: CCc1nn(-c2noc(C3CCN(C(C)C)CC3)n2)c2ccccc12] | 3-ethyl-1-{5-[1- (propan-2- yl)piperidin-4-yl]- 1,2,4-oxadiazol-3- yl}-1H-indazole | ¹H-NMR (DMSO-d₆) δ: 0.97 (6H, d, J = 6.6 Hz), 1.35 (3H, t, J = 7.5 Hz), 1.72- 1.87 (2H, m), 2.06 -2.14 (2H, m), 2.28 (2H, t, J = 10.5 Hz), 2.65-2.78 (1H, m), 2.83 (2H, d, J = 11.6 Hz), 2.98-3.14 (3H, m), 7.37 (1H, t, J = 7.5 Hz), 7.63 (1H, t, J = 7.7 Hz), 7.92 (1H, d, J = 7.9 Hz), 8.19 (1H, d, J = 8.4 Hz). LC-MS, m/z; 340 [M + H] + |
| 145 | 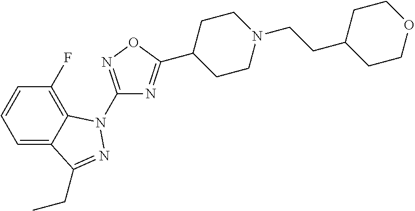 [SMILES: CCc1nn(-c2noc(C3CCN(CCC4CCOCC4)CC3)n2)c2c(F)cccc12] | 3-ethyl-7-fluoro-1- (5-{1-[2- (tetrahydro-2H- pyran-4- yl)ethyl]piperidin- 4-yl}-1,2,4- oxadiazol-3-yl)-1H- indazole | ¹H-NMR (CDCl₃) δ: 1.24-1.68 (10H, m), 2.00-2.25 (6H, m), 2.40 (2H, t, J = 7.7 Hz), 2.91-3.16 (5H, m), 3.32- 3.47 (2H, m), 3.95 (2H, dd, J = 11.3, 3.8 Hz), 7.19-7.31 (2H, m), 7.50-7.57 (1H, m). LC-MS, m/z; 428 [M + H] + |
| 146 | 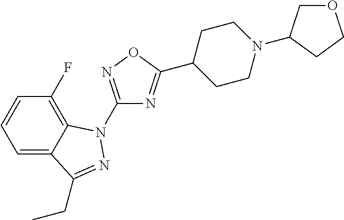 [SMILES: CCc1nn(-c2noc(C3CCN(C4CCOC4)CC3)n2)c2c(F)cccc12] | 3-ethyl-7-fluoro-1- {5-[1- (tetrahydrofuran-3- yl)piperidin-4-yl]- 1,2,4-oxadiazol-3- yl}-1H-indazole | ¹H-NMR (CDCl₃) δ: 1.44 (3H, t, J = 7.6 Hz), 1.81- 1.96 (1H, m), 1.99-2.36 (7H, m), 2.80-2.90 (1H, m), 2.97- 3.16 (5H, m), 3.66 (1H, t, J = 7.8 Hz), 3.80 (1H, dd, J = 16.1, 8.0 Hz), 3.88-4.02 (2H, m), 7.20-7.33 (2H, m), 7.50- 7.59 (1H, m). LC-MS, m/z; 386 [M + H] + |
| 147 | 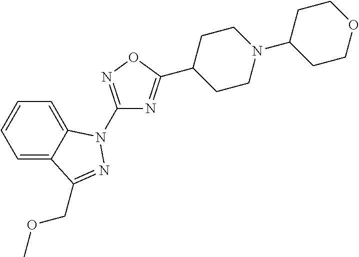 [SMILES: COCc1nn(-c2noc(C3CCN(C4CCOCC4)CC3)n2)c2ccccc12] | 3-(methoxymethyl)- 1-{5-[1- (tetrahydro-2H- pyran-4- yl)piperidin-4-yl]- 1,2,4-oxadiazol-3- yl}-1H-indazole | ¹H-NMR (CDCl₃) δ: 1.55-1.82 (6H, m), 1.99-2.14 (2H, m), 2.17- 2.27 (2H, m), 2.32-2.43 (2H, m), 2.47-2.59 (1H, m), 2.99- 3.12 (3H, m), 3.34-3.49 (5H, m), 4.05 (2H, dd, J = 11.1, 4.3 Hz), 7.36 (1H, t, J = 7.6 Hz), 7.59 (1H, t, J = 7.8 Hz), 7.96 (1H, d, J = 8.0 Hz), 8.30 (1H, d, J = 8.5 Hz). LC-MS, m/z; 398 [M + H] + |
| 148 | 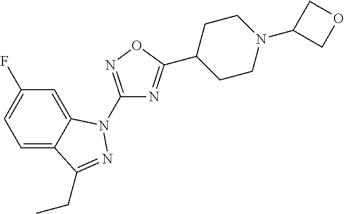 [SMILES: CCc1nn(-c2noc(C3CCN(C4COC4)CC3)n2)c2cc(F)ccc12] | 3-ethyl-6-fluoro-1- {5-[1-(oxetan-3- yl)piperidin-4-yl]- 1,2,4-oxadiazol-3- yl}-1H-indazole | LC-MS, m/z; 372 [M + H] + |
| 149 | 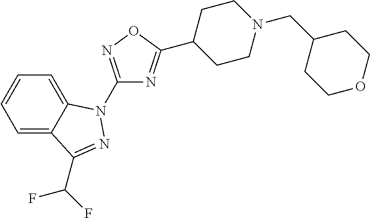 [SMILES: FC(F)c1nn(-c2noc(C3CCN(CC4CCOCC4)CC3)n2)c2ccccc12] | 3-(difluoromethyl)- 1-{5-[1- (tetrahydro-2H- pyran-4- ylmethyl)piperidin- 4-yl]-1,2,4- oxadiazol-3-yl}-1H- indazole | ¹H-NMR (CDCl₃) δ: 1.27 (2H, ddd, J = 24.4, 12.3, 4.0 Hz), 1.56- 1.86 (4H, m), 1.98-2.32 (8H, m), 2.87-3.13 (3H, m), 3.40 (2H, t, J = 11.7 Hz), 3.98 (2H, dd, J = 11.6, 3.3 Hz), 7.45 (1H, t, J = 7.5 Hz), 7.66 (1H, dd, J = 8.4, 7.3 Hz), 8.03 (1H, d, J = 8.1 Hz), 8.36 (1H, d, J = 4.3 Hz). LC-MS, m/z; 418 [M + H] +. |
| 150 | 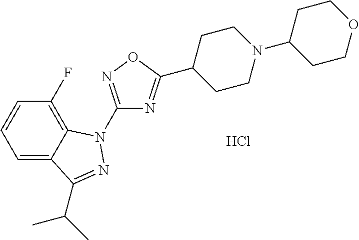 [SMILES: CC(C)c1nn(-c2noc(C3CCN(C4CCOCC4)CC3)n2)c2c(F)cccc12 ; Cl] | 7-fluoro-3-(propan- 2-yl)-1-{5-[1- (tetrahydro-2H- pyran-4- yl)piperidin-4-yl]- 1,2,4-oxadiazol-3- yl}-1H-indazole hydrochloride | ¹H-NMR (DMSO-d₆) δ: 1.41 (6H, d, J = 6.8 Hz), 1.67-1.84 (2H, m), 1.97-2.11 (2H, m), 2.23- 2.47 (4H, m), 3.05-3.68 (9H, m), 3.92-4.06 (2H, m), 7.34- 7.53 (2H, m), 7.80-7.88 (1H, m), 10.89-11.09 (1H, m). LC-MS, m/z; 414 [M + H] + . |
| 151 | 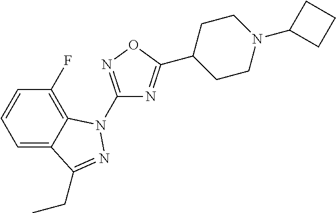 [SMILES: CCc1nn(-c2noc(C3CCN(C4CCC4)CC3)n2)c2c(F)cccc12] | 1-[5-(1-cyclobutyl piperidin-4-yl)- 1,2,4-oxadiazol-3- yl]-3-ethyl-7- fluoro-1H-indazole | ¹H-NMR (DMSO-d₆) δ: 1.34 (3H, t, J = 7.6 Hz), 1.57-1.65 (2H, m), 1.72-1.81 (4H, m), 1.87- 1.99 (4H, m), 2.08 (2H, d, J = 12.0 Hz), 2.69- 2.69 (1H, m), 2.76 (2H, d, J = 11.3 Hz), 3.03 (2H, q, J = 7.6 Hz), 3.11-3.13 (1H, m), 7.34- 7.39 (1H, m), 7.41-7.48 (1H, m), 7.77 (1H, d, J = 8.0 Hz). LC-MS, m/z; 370 [M + H] + |
| 152 | 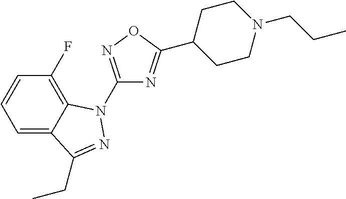 [SMILES: CCCN1CCC(c2nc(-n3nc(CC)c4cccc(F)c43)no2)CC1] | 3-ethyl-7-fluoro-1- [5-(1- propylpiperidin-4- yl)-1,2,4- oxadiazol-3-yl]-1H- indazole | ¹H-NMR (DMSO-d₆) δ: 0.85 (3H, t, J = 7.4 Hz), 1.34 (3H, t, J = 7.6 Hz), 1.44 (2H, m), 1.74- 1.85 (2H, m), 2.03-2.11 (4H, m), 2.24 (2H, t, J = 7.4 Hz), 2.81-2.88 (2H, m), 3.03 (2H, q, J = 7.5 Hz), 3.08-3.17 (1H, m), 7.34-7.39 (1H, m), 7.42- 7.48 (1H, m), 7.77 (1H, d, J = 8.0 Hz). LC-MS, m/z; 358 [M + H] + |
| 153 | 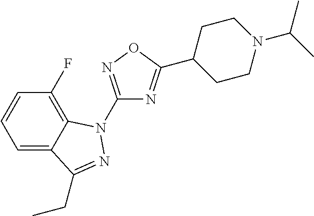 [SMILES: CCc1nn(-c2noc(C3CCN(C(C)C)CC3)n2)c2c(F)cccc12] | 3-ethyl-7-fluoro-1- {5-[1-(propan-2- yl)piperidin-4-yl]- 1,2,4-oxadiazol-3- yl}-1H-indazole | ¹H-NMR (DMSO-d₆) δ: 0.97 (6H d, J = 6.4 Hz), 1.34 (3H, t, J = 7.5 Hz), 1.70- 1.84 (2H, m), 2.04-2.14 (2H, m), 2.27 (2H, t, J = 10.3 Hz), 2.66-2.86 (3H, m), 2.99-3.14 (3H, m), 7.33- 7.48 (2H, m), 7.77 (1H, d, J = 7.5 Hz). LC-MS, m/z; 358 [M + H] + |
| 154 | 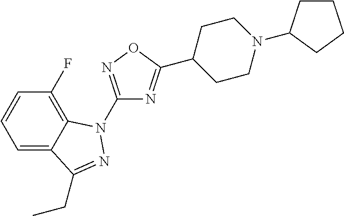 [SMILES: CCc1nn(-c2noc(C3CCN(C4CCCC4)CC3)n2)c2c(F)cccc12] | 1-[5-(1-cyclopentyl piperidin-4-yl)- 1,2,4-oxadiazol-3- yl]-3-ethyl-7- fluoro-1H-indazole | ¹H-NMR (DMSO-d₆) δ: 1.30-1.37 (6H, m), 1.45- 1.62 (4H, m), 1.74-1.85 (4H, m), 2.05-2.15 (4H, m), 2.89- 2.96 (2H, m), 3.03 (2H, q, J = 7.6 Hz), 3.08- 3.17 (1H, m), 7.34-7.39 (1H, m), 7.42-7.47 (1H, m), 7.77 (1H, d, J = 7.8 Hz). LC-MS, m/z; 384 [M + H] + |
| 155 | 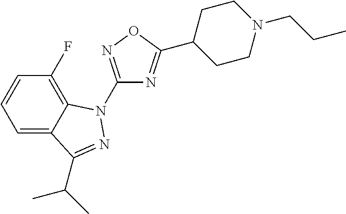 [SMILES: CCCN1CCC(c2nc(-n3nc(C(C)C)c4cccc(F)c43)no2)CC1] | 7-fluoro-3-(propan- 2-yl)-1-[5-(1- propylpiperidin-4- yl)-1,2,4- oxadiazol-3-yl]-1H- indazole | ¹H-NMR (CDCl₃) δ: 0.91 (3H, t, J = 7.3 Hz), 1.45- 1.60 (8H, m), 1.99-2.23 (6H, m), 2.29-2.39 (2H, m), 2.93- 3.12 (3H, m), 3 .41-3.55 (1H, m), 7.17-7.29 (2H, m), 7.55- 7.63 (1H, m). LC-MS, m/z; 372 [M + H] + |
| 156 | 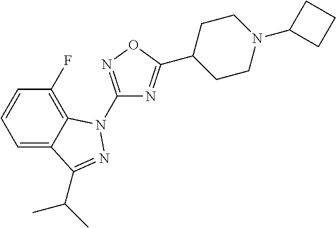 [SMILES: CC(C)c1nn(-c2noc(C3CCN(C4CCC4)CC3)n2)c2c(F)cccc12] | 1-[5-(1-cyclobutyl piperidin-4-yl)- 1,2,4-oxadiazol-3- yl]-7-fluoro-3- (propan-2-yl)-1H- indazole | ¹H-NMR (CDCl₃) δ: 1.50 (6H, d, J = 7.0 Hz), 1.61- 2.25 (12H, m), 2.67-2.81 (1H, m), 2.86-3.12 (3H, m), 3.40- 3.56 (1H, m), 7.17-7.29 (2H, m), 7.54-7.62 (1H, m). LC-MS, m/z; 384 [M + H] + |
| 157 | 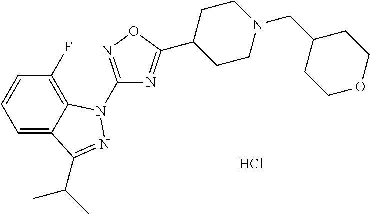 [SMILES: CC(C)c1nn(-c2noc(C3CCN(CC4CCOCC4)CC3)n2)c2c(F)cccc12 ; Cl] | 7-fluoro-3-(propan- 2-yl)-1-{5-[1- (tetrahydro-2H- pyran-4- ylmethyl)piperidin- 4-yl]-1,2,4- oxadiazol-3-yl}-1H- indazole hydrochloride | ¹H-NMR (DMSO-d₆) δ: 1.15-1.33 (2H, m), 1.41 (6H, d, J = 6.8 Hz), 1.69-1.83 (2H, m), 2.00- 2.20 (1H, m), 2.26-2.55 (4H, m), 2.93-3.19 (4H, m), 3.25- 3.56 (5H, m), 3.59-3.76 (1H, m), 3.78-3.93 (2H, m), 7.35- 7.52 (2H, m), 7.81-7.89 (1H, m), 10.34-10.60 (1H, m). LC-MS, m/z; 428 [M + H] + |
| 158 | 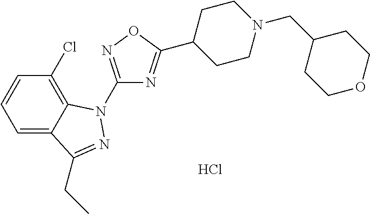 [SMILES: CCc1nn(-c2noc(C3CCN(CC4CCOCC4)CC3)n2)c2c(Cl)cccc12 ; Cl] | 7-chloro-3-ethyl-1- {5-[1-(tetrahydro- 2H-pyran-4- ylmethyl)piperidin- 4-yl]-1,2,4- oxadiazol-3-yl}-1H- indazole hydrochloride | ¹H-NMR (DMSO-d₆) δ: 1.13-1.43 (5H, m), 1.71- 1.87 (2H, m), 2.02-2.19 (1H, m), 2.26-2.53 (4H, m), 2.93- 3.18 (6H, m), 3.25-3.95 (7H, m), 7.39 (1H, t, J = 7.8 Hz), 7.65-7.71 (1H, m), 7.93-8.03 (1H, m), 10.47- 10.74 (1H, m). LC-MS, m/z; 430 [M + H] + |

¹⁾Titanium tetraisopropoxide was added to the reaction system.

### Example 159

### Preparation of 3-ethyl-1-[5-(1-propylazetidin-3-yl)-1,2,4-oxadiazol-3-yl]-1H-indazole trifluoroacetate

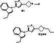 [SMILES: CCc1nn(-c2noc(C3CNC3)n2)c2ccccc12 ; Cl ; CCCN1CC(c2nc(-n3nc(CC)c4ccccc43)no2)C1 ; "CF3COOH"]

1-[5-(Azetidin-3-yl)-1,2,4-oxadiazol-3-yl]-3-ethyl-1H-indazole hydrochloride (100 mg) was suspended in acetonitrile (4 ml). To the suspension were added propyl bromide (48 mg), potassium carbonate (272 mg) and sodium iodide (10 mg), and the mixture was stirred at room temperature overnight. After the reaction was completed, water was added to the reaction solution, and the mixture was extracted with ethyl acetate. The organic layer was washed with water and brine, dried over sodium sulfate and filtered, and the filtrate was concentrated under reduced pressure. The residue was purified by reverse phase HPLC to give the title compound (20 mg) as a pale-yellow oil.

LC-MS, m/z; 312 [M+H]+

The compounds in the following table (i.e. Examples 160 to 165) were prepared in the same manner as in Example 159 except that the 1-[5-(azetidin-3-yl)-1,2,4-oxadiazol-3-yl]-3-ethyl-1H-indazole hydrochloride and propyl bromide were replaced with the corresponding starting compound. In the following table, R—X means an alkylating agent.

TABLE 28

| | | Chemical | | ¹H-NMR/ |
|---|---|---|---|---|
| Ex. | R—X | structure | Compound Name | LC-MS, m/z |
| 160 | 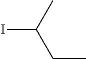 | 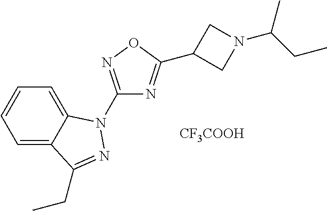 | 1-{5-[1-(butan-2- yl)azetidin-3- yl]-1,2,4- oxadiazol-3-yl}- 3-ethyl-1H- indazole trifluoroacetate | LC-MS, m/z; 326 [M + H] + |
| 161 | 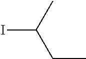 | 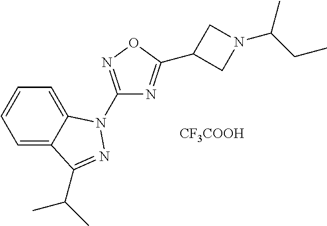 | 1-{5-[1-(butan-2- yl)azetidin-3- yl]-1,2,4- oxadiazol-3-yl}- 3-(propan-2-yl)- 1H-indazole trifluoroacetate | LC-MS, m/z; 340 [M + H] + |
| 162 | 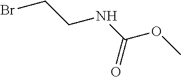 | 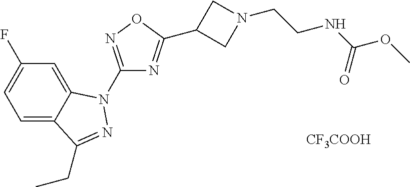 [SMILES: CCc1nn(-c2noc(C3CN(CCNC(=O)OC)C3)n2)c2cc(F)ccc12 ; "CF3COOH"] | methyl (2-{3-[3- (3-ethyl-6- fluoro-1H- indazol-1-yl)- 1,2,4-oxadiazol- 5-yl]azetidin-1- yl}ethyl)carbamate trifluoroacetate | LC-MS, m/z; 389 [M + H] + |
| 163 | 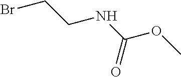 | 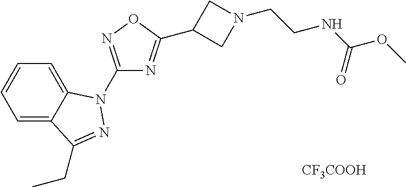 [SMILES: CCc1nn(-c2noc(C3CN(CCNC(=O)OC)C3)n2)c2ccccc12 ; "CF3COOH"] | methyl (2-{3-[3- (3-ethyl-1H- indazol-1-yl)- 1,2,4-oxadiazol- 5-yl]azetidin-1- yl}ethyl)carbamate trifluoroacetate | LC-MS, m/z; 371 [M + H] + |
| 164 | 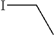 | 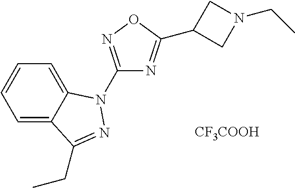 [SMILES: CCc1nn(-c2noc(C3CN(CC)C3)n2)c2ccccc12 ; "CF3COOH"] | 3-ethyl-1-[5-(1- ethylazetidin-3- yl)-1,2,4- oxadiazol-3-yl]- 1H-indazole trifluoroacetate | LC-MS, m/z; 298 [M + H] + |
| 165 | 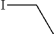 | 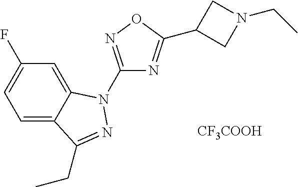 [SMILES: CCc1nn(-c2noc(C3CN(CC)C3)n2)c2cc(F)ccc12 ; "CF3COOH"] | 3-ethyl-1-[5-(1- ethylazetidin-3- yl)-1,2,4- oxadiazol-3-yl]- 6-fluoro-1H- indazole trifluoroacetate | LC-MS, m/z; 316 [M + H] + |

### Preparations of Examples 166 to 167

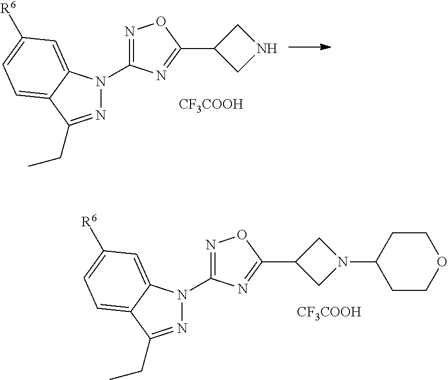

The compounds in the following table (i.e. Examples 166 to 167) were prepared in the same manner as in Example 134 except that the 3-ethyl-6-fluoro-1-[5-(piperidin-4-yl)-1,2,4-oxadiazol-3-yl]-1H-indazole trifluoroacetate was replaced with the corresponding starting compound, the triacetoxysodium borohydride was replaced with sodium cyanoborohydride, and the obtained crude-product was isolated/purified by reverse phase HPLC.

TABLE 29

| | | Chemical | | ¹H-NMR/ |
|---|---|---|---|---|
| Ex. | R⁶ | structure | Compound Name | LC-MS, m/z |
| 166 | H | 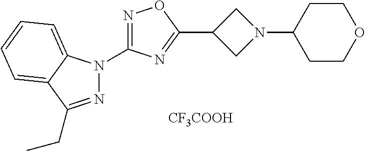 [SMILES: CCc1nn(-c2noc(C3CN(C4CCOCC4)C3)n2)c2ccccc12 ; "CF3COOH"] | 3-ethyl-1-{5-[1- (tetrahydro-2H-pyran-4- yl)azetidin-3-yl]- 1,2,4-oxadiazol-3-yl}- 1H-indazole trifluoroacetate | LC-MS, m/z; 354 [M + H] + |
| 167 | F | 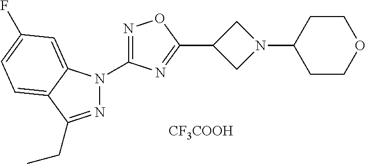 [SMILES: CCc1nn(-c2noc(C3CN(C4CCOCC4)C3)n2)c2cc(F)ccc12 ; "CF3COOH"] | 3-ethyl-6-fluoro-1-{5- [1-(tetrahydro-2H- pyran-4-yl)azetidin-3- yl]-1,2,4-oxadiazol-3- yl}-1H-indazole trifluoroacetate | LC-MS, m/z; 372 [M + H] + |

### Example 168

### Preparation of methyl 4-(2-{4-[3-(3-ethyl-1H-indazol-1-yl)-1,2,4-oxadiazol-5-yl]piperidin-1-yl}ethyl)piperidine-carboxylate hydrochloride

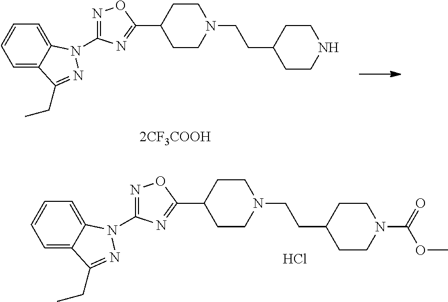 [SMILES: CCc1nn(-c2noc(C3CCN(CCC4CCNCC4)CC3)n2)c2ccccc12 ; CCc1nn(-c2noc(C3CCN(CCC4CCN(C(=O)OC)CC4)CC3)n2)c2ccccc12 ; Cl]

3-Ethyl-1-(5-{1-[2-(piperidin-4-yl)ethyl]piperidin-4-yl}-1,2,4-oxadiazol-3-yl)-1H-indazole bis(trifluoroacetate) (100 mg) was suspended in dichloromethane (4 ml). To the suspension was added triethylamine (38 mg), and the mixture was stirred for 5 minutes. To the reaction mixture was added methyl chloroformate (18 mg), and the mixed solution was stirred at room temperature overnight. After the reaction was completed, water was added to the reaction solution, and the mixture was extracted with ethyl acetate. The organic layer was washed with water and brine, dried over sodium sulfate and filtered, and the filtrate was concentrated under reduced pressure. The residue was purified by silica-gel chromatography (column; Hi-Flash™ Amino Column, developing solvent: hexane/ethyl acetate). The resultant compound was dissolved in methylene chloride and treated with 1N HCl/diethyl ether to give the title compound (33 mg) as a white solid.

LC-MS, m/z; 467 [M+H]+

### Example 169

### Preparation of 3-ethyl-1-[5-(1-{[1-(methylsulfonyl)piperidin-3-yl]methyl}piperidin-4-yl)-1,2,4-oxadiazol-3-yl]-1H-indazole trifluoroacetate

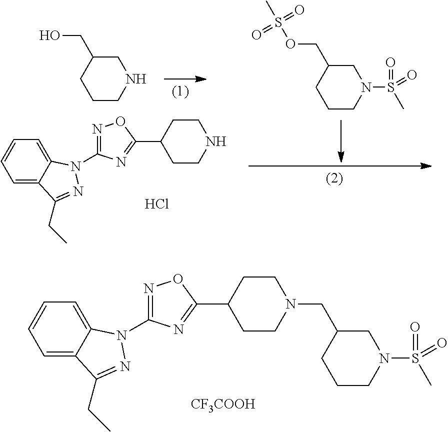

(1) Piperidin-3-ylmethanol (5.0 g) was dissolved in dichloromethane (40 ml). To the solution was added triethylamine (13.2 g), and the mixed solution was stirred at 0° C. To the reaction solution was added dropwise methanesulfonyl chloride (5.97 g) dissolved in dichloromethane (15 ml) at 0° C. with stirring, and the mixture was warmed to room temperature and stirred for 6 hours. Water (30 ml) was added to the reaction solution, and the mixture was extracted with dichloromethane. The organic layer was dried over sodium sulfate and filtered, and the filtrate was concentrated under reduced pressure to give [1-(methylsulfonyl)piperidin-3-yl]methyl methanesulfonate.

(2) 3-Ethyl-1-[5-(piperidin-4-yl)-1,2,4-oxadiazol-3-yl]-1H-indazole hydrochloride (150 mg) was suspended in dichloromethane (4 ml). To the suspension was added triethylamine (58 mg), and the mixture was stirred for 5 minutes. Then the above-prepared [1-(methylsulfonyl)piperidin-3-yl]methyl methanesulfonate (159 mg) was added thereto, and the mixture was stirred at room temperature overnight. After the reaction was completed, water was added to the reaction solution, and the mixture was extracted with ethyl acetate. The organic layer was washed with water and brine, dried over sodium sulfate and filtered, and the filtrate was concentrated under reduced pressure. The residue was purified by reverse phase. HPLC to give the title compound (95 mg) as a pale-yellow oil.

LC-MS, m/z; 473 [M+H]+

### Preparations of Examples 170 to 177

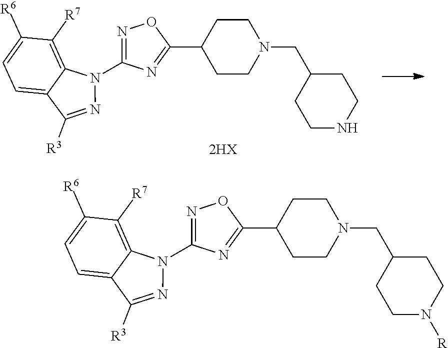

Wherein HX is hydrochloric acid or trifluoroacetic acid.

The compounds in the following table (i.e. Examples 170 to 177) were prepared in the same manner as in Example 168 except that the 3-ethyl-1-(5-{1-[2-(piperidin-4-yl)ethyl]piperidin-4-yl}-1,2,4-oxadiazol-3-yl)-1H-indazole bis(trifluoroacetate) and methyl chloroformate were replaced with the corresponding starting compound and acid chloride (defined as R—Cl), respectively. In order to obtain each of the trifluoroacetates in the following table, the residue was isolated/purified by reverse phase HPLC. Each free form of the compounds in the following table was obtained by omitting the conversion step into hydrochloride in Example 168.

TABLE 30

| | | | | | | ¹H-NMR/ |
|---|---|---|---|---|---|---|
| Ex. | R³ | R⁶ | R⁷ | R | Compound Name | LC-MS, m/z |
| 170 | Et | H | H | —Ms | 3-ethyl-1- | LC-MS, m/z; |
| | | | | | [5-(1-{[1- | 473 [M + H] + |
| | | | | | (methylsulfon- | |
| | | | | | yl)piperidin-4- | |
| | | | | | yl]methyl}piper- | |
| | | | | | idin-4-yl)-1,2,4- | |
| | | | | | oxadiazol-3-yl]- | |
| | | | | | 1H-indazole | |
| | | | | | trifluoroacetate | |
| 171 | Et | F | H | —CO₂Me | methyl 4-({4-[3- | ¹H-NMR |
| | | | | | (3-ethyl-6- | (CDCl₃) |
| | | | | | fluoro-1H- | δ: 1.01-1.16 |
| | | | | | indazol-1-yl)- | (2H, m), 1.44 |
| | | | | | 1,2,4-oxadiazol- | (3H, t, J = 7.6 |
| | | | | | 5-yl]piperidin-1- | Hz), 1.58-1.83 |
| | | | | | yl}methyl)piper- | (3H, m), 1.98- |
| | | | | | idine-1- | 2.24 (8H, m), |
| | | | | | carboxylate | 2.68-3.13 (7H, |
| | | | | | | m), 3.69 (3H, |
| | | | | | | s), 3.99-4.30 |
| | | | | | | (2H, m), 7.08 |
| | | | | | | (1H, td, J = |
| | | | | | | 8.9, 2.2 Hz), |
| | | | | | | 7.70 (1H, dd, J = |
| | | | | | | 8.8, 5.1 Hz), |
| | | | | | | 7.98 (1H, dd, J = |
| | | | | | | 9.3, 2.2 Hz). |
| | | | | | | LC-MS, m/z; 471 |
| | | | | | | [M + H] + |
| 172 | Et | H | F | —CO₂Me | methyl 4-({4-[3- | ¹H-NMR |
| | | | | | (3-ethyl-7- | (CDCl₃) |
| | | | | | fluoro-1H- | δ: 1.00-1.15 |
| | | | | | indazol-1-yl)- | (2H, m), 1.44 |
| | | | | | 1,2,4-oxadiazol- | (3H, t, J = 7.6 |
| | | | | | 5-yl]piperidin-1- | Hz), 1.59-1.81 |
| | | | | | yl}methyl)piper- | (3H, m), 1.96- |
| | | | | | idine-1- | 2.24 (8H, m), |
| | | | | | carboxylate | 2.68-2.82 (2H, |
| | | | | | | m), 2.86-3.13 |
| | | | | | | (5H m), 3.69 |
| | | | | | | (3H, s), 4.00- |
| | | | | | | 4.27 (2H, m), |
| | | | | | | 7.20-7.30 (2H, |
| | | | | | | m), 7.50-7.57 |
| | | | | | | (1H, m). |
| | | | | | | LC-MS, m/z; 471 |
| | | | | | | [M + H] + |
| 173 | ⁱPr | H | F | —CO₂Me | methyl 4-[(4-{3- | ¹H-NMR |
| | | | | | [7-fluoro-3- | (CD₃OD) |
| | | | | | (propan-2-yl)-1H- | δ: 1.16-1.29(3H, |
| | | | | | indazol-1-yl]- | m), 1.48 (6H, d, |
| | | | | | 1,2,4-oxadiazol- | J = 7.0Hz), 1.25- |
| | | | | | 5-yl}piperidin-1- | 3.25 (8H, m), |
| | | | | | yl)methyl]piper- | 3.34-4.22 (10H, |
| | | | | | idine-1- | m), 3.68 (3H, |
| | | | | | carboxylate | s), 7.31-7.36 |
| | | | | | hydrochloride | (2H, m), 7.72- |
| | | | | | | 7.75 (1H, m). |
| | | | | | | LC-MS, m/z; 485 |
| | | | | | | [M + H] + |
| 174 | ⁱPr | H | F | 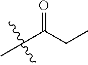 [SMILES: *C(=O)CC] | 1-{4-[(4-{3-[7- fluoro-3- (propan- 2-yl)-1H- indazol- 1-yl]-1,2,4- oxadiazol-5- yl}piperidin-1- yl)methyl] piperidin-1- yl}propan-1- one | ¹H-NMR (CDCl₃) δ: 1.06-1.10 (3H, m), 1.15 (3H, t, J = 7.4 Hz), 1.50 (6H, d, J = 7.0Hz), 1.7-1.9(3H, m), 2.0-2.30 (7H, m), 2.25-2.40 (2H, m), 2.50- 2.65(1H, m), 2.80-3.10(4H, m), 3.40-3.50 (1H, m), 3.80- 3.98 (1H, m), 4.50-4.70(1H, m), 7.14-7.30 (2H, m), 7.58- 7.61 (1H, m). LC-MS, m/z; 483 [M + H] + |
| 175 | Et | H | F | —Ms | 3-ethyl-7-fluoro- 1-[5-(1-{[1- (methylsulfon- yl)piperidin-4- yl]methyl}piper- idin-4-yl)-1,2,4- oxadiazol-3-yl]- 1H-indazole | ¹H-NMR (DMSO-d₆) δ: 1.06-1.16 (2H, m), 1.34 (3H, t, J = 7.6 Hz), 1.58-1.66 (1H, m), 1.75- 1.86 (4H, m), 2.04-2.13 (4H, m), 2.17 (2H, d, J = 7.3 Hz), 2.63-2.71 (2H, m), 2.82-2.86 (2H, m), 2.82 (3H, s), 3.03 (2H, q, J = 7.6 Hz), 3.10-3.18 (1H, m), 3.53 (2H, d, J = 11.7 Hz), 7.34-7.40 (1H, m), 7.42- 7.48 (1H, m), 7.77 (1H, d, J = 7.3 Hz). LC-MS, m/z; 491 [M + H] + |
| 176 | Et | H | F | 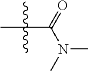 [SMILES: *C(=O)N(C)C] | 4-({4-[3-(3- ethyl-7-fluoro- 1H-indazol-1-yl)- 1,2,4-oxadiazol- 5-yl]piperidin-1- yl}methyl)-N,N- dimethyl- piperidine- 1-carboxamide | ¹H-NMR (DMSO-d₆) δ: 0.97-1.06 (2H, m), 1.34 (3H, t, J = 7.6 Hz), 1.67 (3H, d, J = 10.5 Hz), 1.75-1.85 (2H, m), 2.04-2.16 (6H, m), 2.63- 2.70 (8H, m), 2.80-2.85 (2H, m), 3.03 (2H, q, J = 7.6 Hz), 3.10-3.20 (1H, m), 3.51 (2H, d, J = 12.4 Hz), 7.34-7.40 (1H, m), 7.41-7.48 (1H, m), 7.77 (1H, d, J = 7.3 Hz). LC-MS, m/z; 484 [M + H] + |
| 177 | Et | H | F | —CO₂Et | ethyl 4-({4-[3- (3-ethyl-7- fluoro-1H- indazol-1-yl)- 1,2,4-oxadiazol- 5-yl]piperidin-1- yl}methyl)piper- idine-1- carboxylate | ¹H-NMR (DMSO-d₆) δ: 0.94 (2H, q, J = 11.5 Hz), 1.15 (3H, t, J = 7.1 Hz), 1.34 (3H, t, J = 7.6 Hz), 1.65-1.85 (5H, m), 2.05- 2.14 (6H, m), 2.73 (4H, d, J = 60.7 Hz), 3.03 (2H, q, J = 7.6 Hz), 3.09-3.18 (1H, m), 3.92- 4.03 (4H, m), 7.34-7.39 (1H, m), 7.37 (1H, td, J = 7.8, 4.1 Hz), 7.77 (1H, d, J = 7.8 Hz). LC-MS, m/z; 485 [M + H] + |

### Preparations of Examples 178 to 185

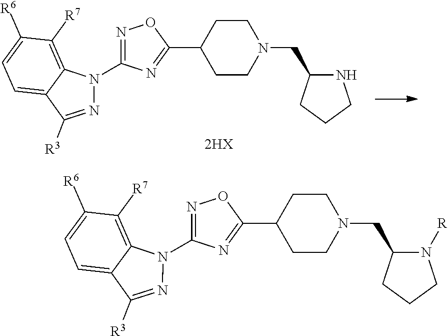

Wherein HX is hydrochloric acid or trifluoroacetic acid.

The compounds in the following table (i.e. Examples 178 to 185) were prepared in the same manner as in Example 168 except that the 3-ethyl-1-(5-{1-[2-(piperidin-4-yl)ethyl]piperidin-4-yl}-1,2,4-oxadiazol-3-yl)-1H-indazole bis(trifluoroacetate) and methyl chloroformate were replaced with the corresponding starting compound and acid chloride (defined as R—Cl) or acetic anhydride, respectively. Each free form of the compounds in the following table was obtained by omitting the conversion step into hydrochloride in Example 168.

TABLE 31

| | | | | | | ¹H-NMR/ |
|---|---|---|---|---|---|---|
| Ex. | R³ | R⁶ | R⁷ | R | Compound Name | LC-MS, m/z |
| 178 | Et | H | H | —CO₂Me | methyl (2S)-2- ({4-[3-(3-ethyl- 1H-indazol-1-yl)- 1,2,4-oxadiazol- 5-yl]piperidin-1- yl}methyl)pyrrolidine- 1-carboxylate | LC-MS, m/z; 439 [M + H]+ |
| 179 | Me | F | H | —CO₂Me | methyl (2S)-2- ({4-[3-(6-fluoro- 3-methyl-1H- indazol-1-yl)- 1,2,4-oxadiazol- 5-yl]piperidin-1- yl}methyl)pyrrolidine- 1-carboxylate hydrochloride | LC-MS, m/z; 443 [M + H]+ |
| 180 | Et | F | H | —CO₂Me | methyl (2S)-2- ({4-[3-(3-ethyl- 6-fluoro-1H- indazol-1-yl)- 1,2,4-oxadiazol- 5-yl]piperidin-1- yl}methyl)pyrrolidine- 1-carboxylate | LC-MS, m/z; 457 [M + H]+ |
| 181 | Et | H | F | —Ac | 1-[(2S)-2-({4-[3- (3-ethyl-7- fluoro-1H- indazol-1-yl)- 1,2,4-oxadiazol- 5-yl]piperidin-1- yl}methyl)pyrrolidin- 1-yl]ethanone | LC-MS, m/z; 441 [M + H]+ |
| 182 | Et | H | F | 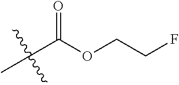 [SMILES: *C(=O)OCCF] | 2-fluoroethyl (2S)-2-({4-[3-(3- ethyl-7-fluoro- 1H-indazol-1-yl)- 1,2,4-oxadiazol- 5-yl]piperidin-1- yl}methyl)pyrrolidine- 1-carboxylate | ¹H-NMR (DMSO- d₆) δ: 1.34 (3H, t, J = 7.6 Hz), 1.72-1.91 (6H, m), 2.01-2.17 (3H, m), 2.18-2.31 (2H, m), 2.41 (1H, t, J = 13.0 Hz), 2.81 (1H, d, J = 10.8 Hz), 2.96 (1H, d, J = 10.8 Hz), 3.02 (2H, q, J = 7.8 Hz), 3.13 (1H, m), 3.21-3.31 (2H, brm), 3.87 (1H, s), 4.20 (2H, d, J = 29.5 Hz), 4.58 (2H, d, J = 47.8 Hz), 7.36 (1H, m), 7.44 (1H, m), 7.76 (1H, d, J = 8.0 Hz). LC-MS, m/z; 489 [M + H]+ |
| 183 | Et | H | F | —CO₂Me | methyl (2S)-2- ({4-[3-(3-ethyl- 7-fluoro-1H- indazol-1-yl)- 1,2,4-oxadiazol- 5-yl]piperidin-1- yl}methyl)pyrrolidine- 1-carboxylate | ¹H-NMR (CDCl₃) δ: 1.44 (3H, t, J = 7.6 Hz), 1.80-2.40 (12H, m), 2.88 (1H, d, J = 10.6 Hz), 2.95-3.22 (4H, m), 3.26-3.46 (2H, m), 3.69 (3H, s), 3.97 (1H, m), 7.19-7.32 (2H, m), 7.53 (1H, m). LC-MS, m/z; 457 [M + H]+ |
| 184 | ⁱPr | H | F | —CO₂Me | methyl (2S)-2- [(4-{3-[7-fluoro- 3-(propan-2-yl)- 1H-indazol-1-yl]- 1,2,4-oxadiazol- 5-yl}piperidin-1- yl)methyl]pyrrolidine- 1-carboxylate | ¹H-NMR (CDCl₃) δ: 1.50 (6H, d, J = 7.0), 1.45- 1.55 (3H, m), 1.80-3.10 (12H, m), 3.28-3.43 (2H, m), 3.46-3.51 (1H, m), 3.70 (3H, s), 3.91-4.03 (1H, m), 7.18-7.32 (2H, m), 7.57-7.61 (1H, m). LC-MS, m/z; 471 [M + H]+ |
| 185 | Et | H | F | —Ms | 3-ethyl-7-fluoro- 1-[5-(1-{[(2S)-1- (methylsulfonyl) pyrrolidin-2- yl]methyl}piperidin- 4-yl)-1,2,4- oxadiazol-3-yl]- 1H-indazole | ¹H-NMR (CDCl₃) δ: 1.44 (3H, t, J = 7.6 Hz), 1.88-2.38 (11H, m), 2.59 (1H, m), 2.93 (3H, s), 2.96-3.14 (5H, m), 3.33-3.43 (2H, m), 3.91 (1H, m), 7.19-7.31 (2H, m), 7.53 (1H, m). LC-MS, m/z; 477 [M + H]+ |

### Preparations of Examples 186 to 190

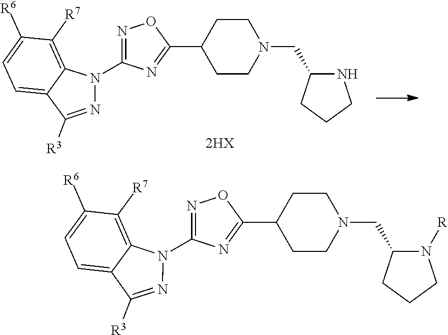

Wherein HX is hydrochloric acid or trifluoroacetic acid.

The compounds in the following table (i.e. Examples 186 to 190) were prepared in the same manner as in Example 168 except that the 3-ethyl-1-(5-{1-[2-(piperidin-4-yl)ethyl]piperidin-4-yl}-1,2,4-oxadiazol-3-yl)-1H-indazole bis(trifluoroacetate) and methyl chloroformate were replaced with the corresponding starting compound and acid chloride (defined as R—Cl) or acetic anhydride, respectively. Each free form of the compounds in the following table was obtained by omitting the conversion step into hydrochloride in Example 168.

TABLE 32

| Ex. | R³ | R⁶ | R⁷ | R | Compound Name | ¹H-NMR/LC-MS, m/z |
|---|---|---|---|---|---|---|
| 186 | Et | H | H | —CO₂Me | methyl (2R)-2- ({4-[3-(3-ethyl- 1H-indazol-1-yl)- 1,2,4-oxadiazol- 5-yl]piperidin-1- yl}methyl)pyrrolidine- 1-carboxylate | ¹H-NMR (DMSO-d₆) δ: 1.35 (3H, t, J = 7.6 Hz), 1.75-1.90 (6H, m), 2.05-2.19 (3H, m), 2.20-2.35 (2H, m), 2.80-2.85 (1H, m), 2.93-3.06 (3H, m), 3.08-3.16 (1H, m), 3.21-3.30 (2H, m), 3.44-3.50 (1H, m), 3.56 (3H, s), 3.87 (1H, s), 7.38 (1H, m), 7.64- 7.64 (1H, m), 7.94 (1H, d, J = 8.1 Hz), 8.20 (1H, d, J = 8.5 Hz). LC-MS, m/z; 439 [M + H]+ |
| 187 | Et | H | F | —Ac | 1-[(2R)-2-({4-[3- (3-ethyl-7- fluoro-1H- indazol-1-yl)- 1,2,4-oxadiazol- 5-yl]piperidin-1- yl}methyl)pyrrolidin- 1-yl]ethanone | ¹H-NMR (CDCl₃) δ: 1.44 (3H, t, J = 7.6 Hz), 1.95-2.59 (13H, m), 2.85-3.24 (6H, m), 3.34-3.75 (4H, m), 7.20-7.31 (2H, m), 7.54 (1H, m). LC-MS, m/z; 441 [M + H]+ |
| 188 | Et | H | F | —CO₂Me | methyl (2R)-2- ({4-[3-(3-ethyl- 7-fluoro-1H- indazol-1-yl)- 1,2,4-oxadiazol- 5-yl]piperidin-1- yl}methyl)pyrrolidine- 1-carboxylate hydrochloride | LC-MS, m/z; 457 [M + H]+ |
| 189 | Et | H | F | 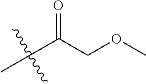 [SMILES: *C(=O)COC] | l-[(2R)-2-({4-[3- (3-ethyl-7- fluoro-1H- indazol-1-yl)- 1,2,4-oxadiazol- 5-yl]piperidin-1- yl}methyl)pyrrolidin- 1-yl]-2- methoxyethanone | LC-MS, m/z; 471 [M + H]+ |
| 190 | Et | H | F | —Ms | 3-ethyl-7-fluoro- 1-[5-(1-{[(2R)-1- (methylsulfonyl) pyrrolidin-2- yl]methyl}piperidin- 4-yl)-1,2,4- oxadiazol-3-yl]- 1H-indazole | ¹H-NMR (CDCl₃) δ: 1.44 (3H, t, J = 7.6 Hz), 1. 88-2.38 (11H, m), 2.59 (1H, m), 2.93 (3H, s), 2.96-3.14 (5H, m), 3.33-3.43 (2H, m), 3.91 (1H, m), 7.19- 7.31 (2H, m), 7.53 (1H, m). LC-MS, m/z; 477 [M + H]+ |

### Preparations of Examples 191 to 203

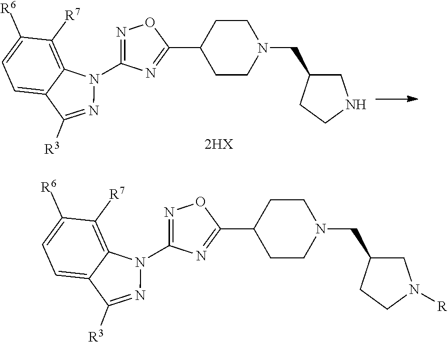

Wherein HX is hydrochloric acid or trifluoroacetic acid.

The compounds in the following table (i.e. Examples 191 to 203) were prepared in the same manner as in Example 168 except that the 3-ethyl-1-(5-{1-[2-(piperidin-4-yl)ethyl]piperidin-4-yl}-1,2,4-oxadiazol-3-yl)-1H-indazole bis(trifluoroacetate) and methyl chloroformate were replaced with the corresponding starting compound and acid chloride (defined as R—Cl) or acetic anhydride, respectively. Each free form of the compounds in the following table was obtained by omitting the conversion step into hydrochloride in Example 168.

TABLE 33

| | | | | | | ¹H-NMR/ |
|---|---|---|---|---|---|---|
| Ex. | R³ | R⁶ | R⁷ | R | Compound Name | LC-MS, m/z |
| 191 | Et | H | H | —Ac | 1-[(3S)-3-({4-[3-(3- ethyl-1H-indazol-1- yl)-1,2,4-oxadiazol- 5-yl]piperidin-1- yl}methyl)pyrrolidin- 1-yl]ethanone | LC-MS, m/z; 423 [M + H]+ |
| 192 | Et | H | F | —CO₂Me | methyl (3S)-3-({4-[3- (3-ethyl-7-fluoro-1H- indazol-1-yl)-1,2,4- oxadiazol-5- yl]piperidin-1- yl}methyl)pyrrolidine- 1-carboxylate | ¹H-NMR (CDCl₃) δ: 1.44 (3H, t, J = 7.7 Hz), 1.55-1.80 (1H, m), 1.93-2.24 (7H, m), 2.28-2.51 (3H, m), 2.87-3.17 (6H, m), 3.27-3.65 (3H, m), 3.70 (3H, s), 7.20-7.32 (2H, m), 7.50-7.58 (1H, m). LC-MS, m/z; 457 [M + H]+ |
| 193 | Et | F | H | —Ac | 1-[(3S)-3-({4-[3-(3- ethyl-6-fluoro-1H- indazol-1-yl)-1,2,4- oxadiazol-5- yl]piperidin-1- yl}methyl)pyrrolidin- 1-yl]ethanone hydrochloride | ¹H-NMR (DMSO- d₆) δ: 1.36 (3H, t, J = 7.6 Hz), 1.57-1.85 (1H, m), 1.88-2.00 (3H, m), 2.02-2.24 (1H, m), 2.26-2.55 (4H, m), 2.58-2.84 (1H, m), 2.92-3.25 (7H, m), 3.27-3.82 (6H, m), 7.26-7.36 (1H, m), 7.86-7.97 (1H, m), 7.99-8.07 (1H, m), 10.62-11.10 (1H, m). LC-MS, m/z; 441 [M + H]+ |
| 194 | Et | F | H | —CO₂Me | methyl (3S)-3-({4-[3- (3-ethyl-6-fluoro-1H- indazol-1-yl)-1,2,4- oxadiazol-5- yl]piperidin-1- yl}methyl)pyrrolidine- 1-carboxylate hydrochloride | ¹H-NMR (DMSO- d₆) δ: 1.36 (3H, t, J = 7.6 Hz), 1.60-1.81 (1H, m), 2.01-2.21 (1H, m), 2.26-2.54 (4H, m), 2.59-2.80 (1H, m), 2.96-3.31 (8H, m), 3.35-3.77 (8H, m), 7.26-7.36 (1H, m), 7.87-8.10 (2H, m), 10.60-10.88 (1H, m). LC-MS, m/z; 457 [M + H]+ |
| 195 | Et | H | H | 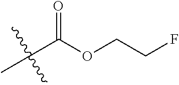 [SMILES: *C(=O)OCCF] | 2-fluoroethyl (3S)-3- ({4-[3-(3-ethyl-1H- indazol-1-yl)-1,2,4- oxadiazol-5- yl]piperidin-1- yl}methyl)pyrrolidine- 1-carboxylate | LC-MS, m/z; 471 [M + H]+ |
| 196 | ⁱPr | H | F | —Ac | l-{(3S)-3-[(4-{3-[7- fluoro-3-(propan-2- yl)-1H-indazol-1-yl]- 1,2,4-oxadiazol-5- yl}piperidin-1- yl)methyl]pyrrolidin- 1-yl}ethanone hydrochloride | ¹H-NMR (CDCl₃) δ: 1.50 (6H, d, J = 7.0 Hz), 2.0-2.60 (13H, m), 2.90-3.75 (9H, m), 7.20-7.28 (2H, m), 7.58-7.61 (1H, m). LC-MS, m/z; 455 [M + H]+ |
| 197 | ⁱPr | H | F | —CO₂Me | methyl (3S)-3-[(4-{3- [7-fluoro-3-(propan- 2-yl)-1H-indazol-1- yl]-1,2,4-oxadiazol- 5-yl}piperidin-1- yl)methyl]pyrrolidine- 1-carboxylate hydrochloride | ¹H-NMR (CD₃OD) δ: 1.47 (6H, d, J = 7.0 Hz), 2.15-3.10 (8H, m), 3.29-3.81 (11H, m), 3.69 (3H, s), 7.30-7.36 (2H, m), 7.71-7.74 (1H, m). LC-MS, m/z; 471 [M + H]+ |
| 198 | ⁱPr | H | F | 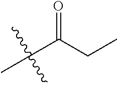 [SMILES: *C(=O)CC] | 1-{(3S)-3-[(4-{3-[7- fluoro-3-(propan-2- yl)-1H-indazol-1-yl]- 1,2,4-oxadiazol-5- yl}piperidin-1- yl)methyl]pyrrolidin- 1-yl}propan-1-one hydrochloride | ¹H-NMR (CD₃OD) δ: 1.09-1.15 (4H, m), 1.48 (6H, d, J = 7.0 Hz), 2.10- 2.70 (7H, m), 3.10-3.35 (5H, m), 3.47-3.54 (3H, m), 3.60-3.75 (2H, m), 3.78- 3.90 (3H, m), 7.30-7.36 (2H, m), 7.70-7.77 (1H, m). LC-MS, m/z; 469 [M + H]+ |
| 199 | ⁱPr | H | F | 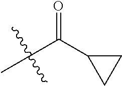 [SMILES: *C(=O)C1CC1] | cyclopropyl{(3S)-3- [(4-{3-[7-fluoro-3- (propan-2-yl)-1H- indazol-1-yl]-1,2,4- oxadiazol-5- yl}piperidin-1- yl)methyl]pyrrolidin- 1-yl}methanone hydrochloride | ¹H-NMR (CD₃OD) δ: 0.80-0.90 (4H, m), 1.47 (6H, d, J = 7.0 Hz), 1.80-2.58 (9H, m), 3.26-3.85 (11H, m), 7.30-7.36 (2H, m), 7.71-7.74 (1H, m). LC-MS, m/z; 481 [M + H]+ |
| 200 | ⁱPr | H | F | 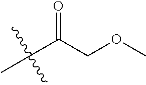 [SMILES: *C(=O)COC] | 1-{(3S)-3-[(4-{3-[7- fluoro-3-(propan-2- yl)-1H-indazol-1-yl]- 1,2,4-oxadiazol-5- yl}piperidin-1- yl)methyl]pyrrolidin- 1-yl}-2- methoxyethanone | ¹H-NMR (CDCl₃) δ: 1.30-1.48 (4H, m), 1.50 (6H, d, J = 6.8 Hz), 1.93-2.55 (6H, m), 3.20-4.00 (11H, m), 4.06 (3H, s), 7.25-7.31 (2H, m), 7.61-7.63 (1H, m). LC-MS, m/z; 485 [M + H]+ |
| 201 | Et | H | F | —Ac | 1-[(3S)-3-({4-[3-(3- ethyl-7-fluoro-1H- indazol-1-yl)-1,2,4- oxadiazol-5- yl]piperidin-1- yl}methyl)pyrrolidin- 1-yl]ethanone | ¹H-NMR (CDCl₃) δ: 1.44 (3H, t, J = 7.6 Hz), 1.95-2.59 (13H, m), 2.85-3.24 (6H, m), 3.34-3.75 (4H, m), 7.20-7.31 (2H, m), 7.54 (1H, m). LC-MS, m/z; 441 [M + H]+ |
| 202 | Et | H | F | 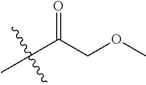 [SMILES: *C(=O)COC] | 1-[(3S)-3-({4-[3-(3- ethyl-7-fluoro-1H- indazol-1-yl)-1,2,4- oxadiazol-5- yl]piperidin-1- yl}methyl)pyrrolidin- 1-yl]-2- methoxyethanone | ¹H-NMR (CDCl3) δ: 1.36-1.83 (4H, m), 1.92-2.61 (10H, m), 2.85-3.81 (12H, m), 4.04 (2H, d, J = 3.5 Hz), 7.19-7.34 (2H, m), 7.50-7.59 (1H, m). LC-MS, m/z; 471 [M + H]+. |
| 203 | Et | H | F | —Ms | 3-ethyl-7-fluoro-1- [5-(1-{[(3S)-1- (methylsulfonyl) pyrrolidin-3- yl]methyl}piperidin- 4-yl)-1,2,4- oxadiazol-3-yl]-1H- indazole | ¹H-NMR (CDCl₃) δ: 1.44 (3H, t, J = 7.6 Hz), 1.71 (1H, m), 1.96-2.58 (10H, m), 2.84 (3H, s), 2.86-3.15 (6H, m) 3.27- 3.56 (3H, m), 7.20-7.31 (2H, m), 7.54 (1H, m). LC-MS, m/z; 477 [M + H]+ |

### Preparations of Examples 204 to 216

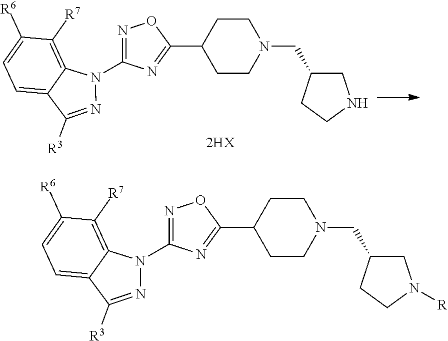

Wherein HX is hydrochloric acid or trifluoroacetic acid.

The compounds in the following table (i.e. Examples 204 to 216) were prepared in the same manner as in Example 168 except that the 3-ethyl-1-(5-{1-[2-(piperidin-4-yl)ethyl]piperidin-4-yl}-1,2,4-oxadiazol-3-yl)-1H-indazole bis(trifluoroacetate) and methyl chloroformate were replaced with the corresponding starting compound and acid chloride (defined as R—Cl) or acetic anhydride, respectively. Each free form of the compounds in the following table was obtained by omitting the conversion step into hydrochloride in Example 168.

TABLE 34

| | | | | | | ¹H-NMR/ |
|---|---|---|---|---|---|---|
| Ex. | R³ | R⁶ | R⁷ | R | Compound Name | LC-MS, m/z |
| 204 | Et | H | F | —CO₂Me | methyl (3R)-3-({4- [3-(3-ethyl-7- fluoro-1H-indazol- 1-yl)-1,2,4- oxadiazol-5- yl]piperidin-1- yl}methyl)pyrrolidine- 1-carboxylate | ¹H-NMR (CDCl₃) δ: 1.44 (3H, t, J = 7.7 Hz), 1.54-1.73 (1H, m), 1.92-2.25 (7H, m), 2.27-2.50 (3H, m), 2.87-3.17 (6H, m), 3.27-3.66 (3H, m), 3.70 (3H, s), 7.20-7.32 (2H, m), 7.50-7.58 (1H, m). LC-MS, m/z; 457 [M + H]+ |
| 205 | Et | F | H | —Ac | 1-[(3R)-3-({4-[3- (3-ethyl-6-fluoro- 1H-indazol-1-yl)- 1,2,4-oxadiazol-5- yl]piperidin-1- yl}methyl)pyrrolidin- 1-yl]ethanone hydrochloride | ¹H-NMR (DMSO-d₆) δ: 1.36 (3H, t, J = 7.6 Hz), 1.57- 1.84 (1H, m), 1.87-1.99 (3H, m), 2.02-2.54 (5H, m), 2.58- 2.86 (1H, m), 2.91-3.26 (7H, m), 3.27-3.82 (6H, m), 7.23- 7.37 (1H, m), 7.83-8.10 (2H, m), 10.58-11.07 (1H, m). LC-MS, m/z; 441 [M + H]+ |
| 206 | Et | F | H | —CO₂Me | methyl (3R)-3-({4- [3-(3-ethyl-6- fluoro-1H-indazol- 1-yl)-1,2,4- oxadiazol-5- yl]piperidin-1- yl}methyl)pyrrolidine- 1-carboxylate hydrochloride | ¹H-NMR (DMSO-d₆) δ: 1.36 (3H, t, J = 7.6 Hz), 1.59- 1.82 (1H, m), 2.04-2.19 (1H, m), 2.26-2.48 (4H, m), 2.61- 2.82 (1H, m), 2.98-3.29 (8H, m,) 3.33-3.81 (8H, m), 7.26- 7.36 (1H, m), 7.86-8.11 (2H, m), 10.57-10.88 (1H, m). LC-MS, m/z; 457 [M + H]+ |
| 207 | Et | H | H | 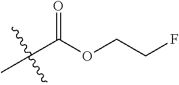 [SMILES: *C(=O)OCCF] | 2-fluoroethyl (3R)-3-({4-[3-(3- ethyl-1H-indazol- 1-yl)-1,2,4- oxadiazol-5- yl]piperidin-1- yl}methyl)pyrrolidine- 1-carboxylate | LC-MS, m/z; 471 [M + H]+ |
| 208 | Et | H | H | —Ac | 1-[(3R)-3-({4-[3- (3-ethyl-1H- indazol-1-yl)- 1,2,4-oxadiazol-5- yl]piperidin-1- yl}methyl)pyrrolidin- 1-yl]ethanone | LC-MS, m/z; 423 [M + H]+ |
| 209 | Et | H | F | —Ac | 1-[(3R)-3-({4-[3- (3-ethyl-7-fluoro- 1H-indazol-1-yl)- 1,2,4-oxadiazol-5- yl]piperidin-1- yl}methyl)pyrrolidin- 1-yl]ethanone hydrochloride | ¹H-NMR (DMSO-d₆) δ: 1.36 (3H, t, J = 7.5 Hz), 1.57- 1.87 (1H, m), 1.89-2.01 (3H, m), 2.02-2.55 (5H, m), 2.58- 2.88 (1H, m), 2.91-3.27 (7H, m), 3.28-3.83 (6H, m), 7.34- 7.54 (2H, m), 7.76-7.84 (1H, m), 10.71-11.26 (1H, br m). LC-MS, m/z; 441 [M + H]+ |
| 210 | Et | H | F | 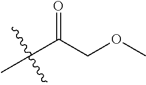 [SMILES: *C(=O)COC] | 1-[(3R)-3-({4-[3- (3-ethyl-7-fluoro- 1H-indazol-1-yl)- 1,2,4-oxadiazol-5- yl]piperidin-1- yl}methyl)pyrrolidin- 1-yl]-2- methoxyethanone hydrochloride | ¹H-NMR (CDCl₃) δ: 1.32-1.52 (3H, m), 1.67-4.29 (25H, m), 7.18-7.34 (2H, m), 7.47-7.61 (1H, m), 11.82-12.56 (1H, br m). LC-MS, m/z; 471 [M + H]+ |
| 211 | ⁱPr | H | F | —Ac | 1-{(3R)-3-[(4-{3- [7-fluoro-3- (propan-2-yl)-1H- indazol-1-yl]- 1,2,4-oxadiazol-5- yl}piperidin-1- yl)methyl]pyrrolidin- 1-yl}ethanone hydrochloride | ¹H-NMR (CD₃OD) δ: 1.47 (6H, d, J = 7.0 Hz), 1.83-2.93 (12H, m), 3.35-4.72 (10H, m), 7.30-7.35 (2H, m), 7.72-7.75 (1H, m). LC-MS, m/z; 455 [M + H]+ |
| 212 | ⁱPr | H | F | —CO₂Me | methyl (3R)-3-[(4- {3-[7-fluoro-3- (propan-2-yl)-1H- indazol-1-yl]- 1,2,4-oxadiazol-5- yl}piperidin-1- yl)methyl]pyrrolidine- 1-carboxylate hydrochloride | ¹H-NMR (CD₃OD) δ: 1.46 (6H, d, J = 7.0 Hz), 2.15-2.86 (6H, m), 3.09-3.80 (13H, m), 3.68 (3H, s), 7.29-7.35 (2H, m), 7.70-7.73 (1H, m). LC-MS, m/z; 471 [M + H]+ |
| 213 | ⁱPr | H | F | 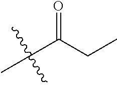 [SMILES: *C(=O)CC] | 1-{(3R)-3-[(4-{3- [7-fluoro-3- (propan-2-yl)-1H- indazol-1-yl]- 1,2,4-oxadiazol-5- yl}piperidin-1- yl)methyl]pyrrolidin- 1-yl}propan-1- one | ¹H-NMR (CDCl₃) δ: 1.16 (3H, t, J = 7.4 Hz), 1.50 (6H, d, J = 7.0 Hz), 2.05-2.36 (12H, m), 2.92-3.20 (4H, m), 3.38-3.70 (5H, m), 7.20- 7.25 (2H, m), 7.57-7.61 (1H, m). LC-MS, m/z; 469 [M + H]+ |
| 214 | ⁱPr | H | F | 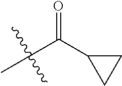 [SMILES: *C(=O)C1CC1] | cyclopropyl{(3R)- 3-[(4-{3-[7- fluoro-3-(propan- 2-yl)-1H-indazol- 1-yl]-1,2,4- oxadiazol-5- yl}piperidin-1- yl)methyl]pyrrolidin- 1-yl}methanone | ¹H-NMR (CDCl₃) δ: 0.74-0.78 (2H, m), 0.94-1.07 (2H, m), 1.50 (6H, d, J = 7.0 Hz), 2.02-2.22 (8H, m), 2.38-3.41 (2H, m), 2.87-3.19 (4H, m), 3.36-3.62 (6H, m), 7.20-7.25 (2H, m), 7.58-7.61 (1H, m). LC-MS, m/z; 481 [M + H]+ |
| 215 | ⁱPr | H | F | 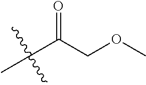 [SMILES: *C(=O)COC] | 1-{(3R)-3-[(4-{3- [7-fluoro-3- (propan-2-yl)-1H- indazol-1-yl]- 1,2,4-oxadiazol-5- yl}piperidin-1- yl)methyl]pyrrolidin- 1-yl}-2- methoxyethanone | ¹H-NMR (CDCl₃) δ: 1.50 (6H, d, J = 7.0 Hz), 2.05-2.55 (9H, m), 2.92-3.22 (5H, m), 3.42- 3.79 (8H, m), 4.04-4.06 (2H, m), 7.20-7.25 (2H, m), 7.58-7.60 (1H, m). LC-MS, m/z; 485 [M + H]+ |
| 216 | Et | H | F | —Ms | 3-ethyl-7-fluoro- 1-[5-(1-{[(3R)-1- (methylsulfonyl) pyrrolidin-3- yl]methyl}piperidin- 4-yl)-1,2,4- oxadiazol-3-yl]- 1H-indazole | ¹H-NMR (CDCl₃) δ: 1.44 (3H, t, J = 7.6 Hz), 1.71 (1H, m), 1.96-2.58 (10H, m), 2.84 (3H, s), 2.86-3.15 (6H, m) 3.27-3.56 (3H, m), 7.20-7.31 (2H, m), 7.54 (1H, m). LC-MS, m/z; 477 [M + H]+ |

### Preparations of Examples 217 to 226

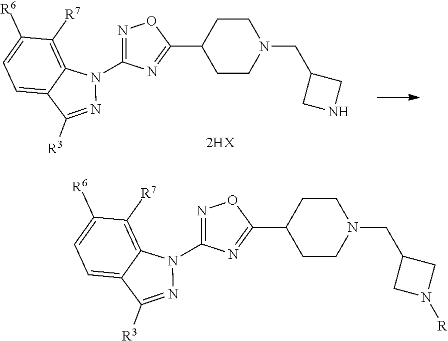

Wherein HX is hydrochloric acid or trifluoroacetic acid.

The compounds in the following table (i.e. Examples 217 to 226) were prepared in the same manner as in Example 168 except that the 3-ethyl-1-(5-{1-[2-(piperidin-4-yl)ethyl]piperidin-4-yl}-1,2,4-oxadiazol-3-yl)-1H-indazole bis(trifluoroacetate) and methyl chloroformate were replaced with the corresponding starting compound and acid chloride (defined as R—Cl) or acetic anhydride, respectively. Each free form of the compounds in the following table was obtained by omitting the conversion step into hydrochloride in Example 168.

TABLE 35

| Ex. | R³ | R⁶ | R⁷ | R | Compound Name | ¹H-NM/LC-MS, m/z |
|---|---|---|---|---|---|---|
| 217 | Et | F | H | —CO₂Me | methyl 3-({4- [3-(3-ethyl-6- fluoro-1H- indazol-1-yl)- 1,2,4- oxadiazol-5- yl]piperidin- 1-yl}methyl) azetidine-1- carboxylate | ¹H-NMR (CDCl₃) δ: 1.44 (3H, t, J = 7.6 Hz), 1.95-2.28 (6H, m), 2.54-2.69 (2H, m), 2.71-3.15 (6H, m), 3.60-3.74 (5H, m), 4.09 (2H, t, J = 8.4 Hz), 7.08 (1H, td, J = 8.8, 2.2 Hz), 7.70 (1H, dd, J = 8.7, 5.0 Hz), 7.97 (1H, dd, J = 9.4, 1.7 Hz). LC-MS, m/z; 443 [M + H]+ |
| 218 | Et | H | F | —CO₂Me | methyl 3-({4- [3-(3-ethyl-7- fluoro-1H- indazol-1-yl)- 1,2,4- oxadiazol-5- yl]piperidin- 1-yl}methyl) azetidine-1- carboxylate | ¹H-NMR (CDCl₃) δ: 1.44 (3H, t, J = 7.6 Hz), 1.96-2.26 (6H, m), 2.61 (2H, d, J = 7.6 Hz), 2.72-2.94 (3H, m), 2.98-3.13 (3H, m), 3.62-3.74 (5H, m), 4.08 (2H, t, J = 8.3 Hz), 7.20-7.30 (2H, m), 7.50-7.56 (1H, m). |
| 219 | ⁱPr | H | F | —Ac | 1-{3-[(4-{3- [7-fluoro-3- (propan-2-yl)- 1H-indazol-1- yl]-1,2,4- oxadiazol-5- yl}piperidin- 1-yl)methyl] azetidin-1- yl}ethanone hydrochloride | ¹H-NMR (DMSO-d₆) δ: 1.41 (6H, d, J = 7.1 Hz), 1.69-1.77 (3H, m), 2.18-2.42 (3H, m), 3.02-3.20 (3H, m), 3.31-3.59 (7H, m), 3.62-3.76 (1H, m), 3.89-4.03 (2H, m), 4.20-4.31 (1H, m), 7.33-7.52 (2H, m), 7.80-7.88 (1H, m), 10.89- 11.11 (1H, m). LC-MS, m/z; 441 [M + H]+ |
| 220 | ⁱPr | H | F | —CO₂Me | methyl 3-[(4 {3-[7-fluoro- 3-(propan-2- yl)-1H- indazol-1-yl]- 1,2,4- oxadiazol-5- yl}piperidin- 1-yl)methyl] azetidine-1- carboxylate hydrochloride | ¹H-NMR (DMSO-d₆) δ: 1.41 (6H, d, J = 6.8 Hz), 2.13-2.41 (4H, m), 3.00-3.20 (3H, m), 3.30-3.59 (9H, m), 3.68-3.86 (2H, m), 3.94-4.13 (2H, m), 7.31-7.53 (2H, m), 7.78-7.89 (1H, m), 10.74- 10.98 (1H, m). LC-MS, m/z; 457 [M + H]+ |
| 221 | ⁱPr | H | F | 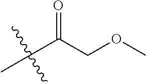 [SMILES: *C(=O)COC] | 1-{3-[(4-{3- [7-fluoro-3- (propan-2-yl)- 1H-indazol-1- yl]-1,2,4- oxadiazol-5- yl}piperidin- 1-yl)methyl] azetidin-1-yl}-2- methoxyethanone hydrochloride | ¹H-NMR (DMSO-d₆) δ: 1.41 (6H, d, J = 6.8 Hz), 2.17-2.45 (4H, m), 3.03-3.60 (12H, m), 3.69- 3.80 (1H, m), 3.85-4.12 (4H, m), 4.26-4.38 (1H, m), 7.35-7.54 (2H, m), 7.82-7.90 (1H, m), 10.86-11.11 (1H, m). LC-MS, m/z; 471 [M + H]+ |
| 222 | ⁱPr | H | F | 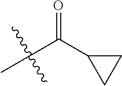 [SMILES: *C(=O)C1CC1] | cyclopropyl{3- [(4-{3-[7- fluoro-3- (propan-2-yl)- 1H-indazol-1- yl]-1,2,4- oxadiazol-5- yl}piperidin- 1-yl)methyl] azetidin-1- yl}methanone hydrochloride | ¹H-NMR (DMSO-d₆) δ: 0.62-0.78 (4.0H, m), 1.36-1.54 (7H, m), 2.17-2.45 (4H, m), 3.04-3.25 (3H, m), 3.35-3.62 (6H, m), 3.64-3.78 (1H, m), 3.93-4.16 (2H, m), 4.34-4.47 (1H, m), 7.35-7.55 (2H, m), 7.81-7.90 (1H, m), 10.82-11.07 (1H, m). LC-MS, m/z; 467 [M + H]+ |
| 223 | Et | H | F | —Ac | 1-[3-({4-[3-(3- ethyl-7-fluoro- 1H-indazol-1- yl)-1,2,4- oxadiazol-5- yl]piperidin-1- yl}methyl) azetidin-1- yl]ethanone hydrochloride | ¹H-NMR (DMSO-d₆) δ: 1.35 (3H, t, J = 7.5 Hz), 1.73 (3H, s), 2.10-2.21 (2H, m), 2.31-2.40 (3H, m), 3.00-3.10 (4H, m), 3.36-3.40 (3H, m), 3.51-3.55 (2H, m), 3.64-3.69 (1H, m), 3.91-4.00 (2H, m), 4.22-4.27 (1H, m), 7.37-7.41 (1H, m), 7.44-7.49 (1H, m), 7.78 (1H, d, J = 7.7 Hz). LC-MS, m/z; 427 [M + H]+ |
| 224 | Et | H | F | 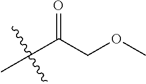 [SMILES: *C(=O)COC] | 1-[3-({4-[3-(3- ethyl-7-fluoro- 1H-indazol-1- yl)-1,2,4- oxadiazol-5- yl]piperidin-1- yl}methyl) azetidin-1-yl]-2- methoxyethanone hydrochloride | ¹H-NMR (CDCl₃) δ: 1.36 (3H, t, J = 7.6 Hz), 2.17-2.46 (4H, m), 3.00-3.24 (5H, m), 3.28 (3H, s), 3.36-3.59 (5H, m), 3.69-3.80 (1H, m), 3.89 (2H, s), 3.95-4.10 (2H, m), 4.27-4.37 (1H, m), 7.36-7.52 (2H, m), 7.77-7.82 (1H, m), 10.85-11.16 (1H, br m). LC-MS, m/z; 457 [M + H]+ |
| 225 | Et | H | F | 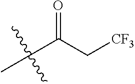 [SMILES: *C(=O)CC(F)(F)F] | 1-[3-({4-[3-(3- ethyl-7-fluoro- 1H-indazol-1- yl)-1,2,4- oxadiazol-5- yl]piperidin-1- yl}methyl) azetidin-1-yl]- 3,3,3- trifluoropropan- 1-one hydrochloride | LC-MS, m/z; 495 [M + H]+ |
| 226 | Et | H | F | —Ms | 3-ethyl-7- fluoro-1-[5-(1- {[1- (methylsulfonyl) azetidin-3- yl]methyl} piperidin-4-yl)- 1,2,4- oxadiazol-3- yl]-1H-indazole | ¹H-NMR (DMSO-d₆) δ: 1.34 (3H, t, J = 7.6 Hz), 1.79 (2H, q, J = 11.0 Hz), 2.05-2.17 (4H, m), 2.54 (2H, d, J = 7.3 Hz), 2.77-2.84 (3H, m), 2.98-3.05 (5H, m), 3.14 (1H, m), 3.55 (2H, t, J = 7.1 Hz), 3.89 (2H, t, J = 8.0 Hz), 7.34-7.40 (1H, m), 7.42-7.48 (1H, m), 7.77 (1H, d, J = 8.0 Hz). LC-MS, m/z; 463 [M + H]+ |

### Preparations of Examples 227 to 241

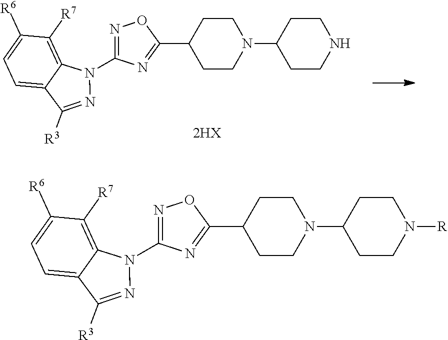

Wherein HX is hydrochloric acid or trifluoroacetic acid.

The compounds in the following table (i.e. Examples 227 to 241) were prepared in the same manner as in Example 168 except that the 3-ethyl-1-(5-{1-[2-(piperidin-4-yl)ethyl]piperidin-4-yl}-1,2,4-oxadiazol-3-yl)-1H-indazole bis(trifluoroacetate) and methyl chloroformate were replaced with the corresponding starting compound and acid chloride (defined as R—Cl) or acetic anhydride, respectively. Each free form of the compounds in the following table was obtained by omitting the conversion step into hydrochloride in Example 168.

TABLE 36

| | | | | | | ¹H-NMR/LC-MS, |
|---|---|---|---|---|---|---|
| Ex. | R³ | R⁶ | R⁷ | R | Compound Name | m/z |
| 227 | Et | H | H | —CO₂Me | methyl 4-[3-(3- ethyl-1H- indazol-1-yl)- 1,2,4-oxadiazol- 5-yl]-1,4′- bipiperidine-1′- carboxylate | LC-MS, m/z; 439 [M + H]+ |
| 228 | Et | F | H | —Ac | 1-{4-[3-(3- ethyl-6-fluoro- 1H-indazol-1- yl)-1,2,4- oxadiazol-5-yl]- 1,4′-bipiperidin-1′- yl}ethanone hydrochloride | ¹H-NMR (DMSO-d₆) δ: 1.36 (3H, t, J = 7.4 Hz), 1.44-1.77 (2H, m), 1.97-2.21 (5H, m), 2.27-2.59 (5H, m), 2.95-3.27 (5H, m), 3.39-3.63 (4H, m), 3.88-4.04 (1H, m), 4.44-4.60 (1H, m), 7.25-7.37 (1H, m), 7.85-8.10 (2H, m), 10.86-11.09 (1H, m). LC-MS, m/z; 441 [M + H]+ |
| 229 | Et | H | F | —Ac | 1-{4-[3-(3- ethyl-7-fluoro- 1H-indazol-1- yl)-1,2,4- oxadiazol-5-yl]- 1,4′-bipiperidin-1′- yl}ethanone hydrochloride | ¹H-NMR (DMSO-d₆) δ: 1.36 (3H, t, J = 7.6 Hz), 1.43-1.76 (2H, m), 1.96-2.20 (5H, m), 2.23-2.57 (5H, m), 2.94-3.27 (5H, m), 3.31-3.61 (4H, m), 3.89-4.04 (1H, m), 4.43-4.59 (1H, m), 7.34-7.53 (2H, m), 7.76-7.83 (1H, m), 10.81-11.00 (1H, m). LC-MS, m/z; 441 [M + H]+ |
| 230 | Et | F | H | —CO₂Me | methyl 4-[3-(3- ethyl-6-fluoro- 1H-indazol-1- yl)-1,2,4- oxadiazol-5-yl]- 1,4′-bipiperidine-1′- carboxylate | ¹H-NMR (CDCl₃) δ: 1.38-1.57 (5H, m), 1.75- 1.87 (2H, m), 1.95-2.26 (4H, m), 2.33-2.58 (3H, m), 2.69- 2.87 (2H, m), 2.95-3.13 (5H, m), 3.70 (3H, s), 4.05-4.36 (2H, m), 7.08 (1H, td, J = 8.8, 2.2 Hz), 7.70 (1H, dd, J = 8.7, 5.0 Hz), 7.98 (1H, dd, J = 9.5, 2.2 Hz). |
| 231 | Et | H | F | —CO₂Me | methyl 4-[3-(3- ethyl-7-fluoro- 1H-indazol-1- yl)-1,2,4- oxadiazol-5-yl]- 1,4′-bipiperidine-1′- carboxylate | ¹H-NMR (CDCl₃) δ: 1.36-1.54 (5H, m), 1.75-1.87 (2H, m), 1.94-2.24 (4H, m), 2.32-2.54 (3H, m), 2.69-2.85 (2H, m), 2.92-3.14 (5H, m), 3.69 (3H, s), 4.04-4.34 (2H, m), 7.19-7.30 (2H, m), 7.49- 7.57 (1H, m). |
| 232 | ⁱPr | H | F | —Ac | 1-(4-{3-[7- fluoro-3- (propan-2-yl)- 1H-indazol-1- yl]-1,2,4- oxadiazol-5-yl}- 1,4′-bipiperidin-1′- yl)ethanone hydrochloride | ¹H-NMR (DMSO-d₆) δ: 1.41 (6H, d, J = 6.8 Hz), 1.45-1.76 (2H, m), 1.96-2.21 (5H, m), 2.24-2.58 (5H, m), 2.96-3.27 (3H, m), 3.34-3.61 (5H, m), 3.89-4.04 (1H, m), 4.44-4.59 (1H, m), 7.34-7.53 (2H, m), 7.81-7.91 (1H, m), 10.86-11.12 (1H, m). LC-MS, m/z; 455 [M + H]+ |
| 233 | ⁱPr | H | F | 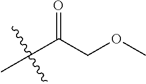 [SMILES: *C(=O)COC] | 1-(4-{3-[7- fluoro-3- (propan-2-yl)- 1H-indazol-1- yl]-1,2,4- oxadiazol-5-yl}- 1,4′-bipiperidin-1′-yl)- 2-methoxyethanone hydrochloride | ¹H-NMR (DMSO-d₆) δ: 1.41 (6H, d, J = 6.8 Hz), 1.48-1.77 (2H, m), 2.07-2.66 (7H, m), 2.91-3.06 (1H, m), 3.10-3.63 (10H, m), 3.83-4.19 (3H, m), 4.41-4.57 (1H, m), 7.34-7.53 (2H, m), 7.80-7.90 (1H, m), 10.67-10.86 (1H, m). LC-MS, m/z; 485 [M + H]+ |
| 234 | ⁱPr | H | F | —CO₂Me | methyl 4-{3-[7- fluoro-3- (propan-2-yl)- 1H-indazol-1- yl]-1,2,4- oxadiazol-5-yl}- 1,4′-bipiperidine-1′- carboxylate hydrochloride | ¹H-NMR (DMSO-d₆) δ: 1.41 (6H, d, J = 6.8 Hz), 1.50-1.70 (2H, m), 2.05-2.19 (2H, m), 2.23-2.53 (4H, m), 2.68-2.93 (2H, m), 3.07-3.27 (2H, m), 3.39-3.63 (8H, m), 4.00-4.20 (2H, m), 7.33-7.52 (2H, m), 7.79-7.88 (1H, m), 10.95-11.12 (1H, m). LC-MS, m/z; 471 [M + H]+ |
| 235 | ⁱPr | H | F | 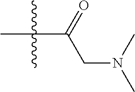 [SMILES: *C(=O)CN(C)C] | 2-(dimethylamino)- 1-(4-{3-[7- fluoro-3-(propan-2-yl)- 1H-indazol-1-yl]-1,2,4- oxadiazol-5-yl}- 1,4′-bipiperidin-1′- yl)ethanone | LC-MS, m/z; 498 [M + H]+ |
| 236 | ⁱPr | H | F | 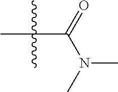 [SMILES: *C(=O)N(C)C] | 4-{3-[7-fluoro- 3-(propan-2-yl)- 1H-indazol-1- yl]-1,2,4- oxadiazol-5-yl}- N,N-dimethyl- 1,4′-bipiperidine-1′- carboxamide hydrochloride | ¹H-NMR (DMSO-d₆) δ: 1.41 (6H, d, J = 7.1 Hz), 1.54-1.75 (2H, m), 2.02-2.16 (2H, m), 2.23-2.48 (4H, m), 2.63-2.84 (8H, m), 3.09-3.27 (2H, m), 3.33-3.72 (7H, m), 7.33-7.54 (2H, m), 7.80-7.89 (1H, m), 10.80-10.95 (1H, m). LC-MS, m/z; 484 [M + H]+ |
| 237 | Et | H | F | 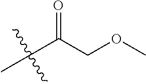 [SMILES: *C(=O)COC] | 1-{4-[3-(3- ethyl-7-fluoro- 1H-indazol-1- yl)-1,2,4- oxadiazol-5-yl]- 1,4′-bipiperidin-1′- yl}-2-methoxyethanone hydrochloride | ¹H-NMR (DMSO-d₆) δ: 1.36 (3H, t, J = 7.5 Hz), 1.43-1.77 (2H, m), 2.04-2.66 (7H, m), 2.91-3.65 (12H, m), 3.84-4.22 (3H, m), 4.41-4.56 (1H, m), 7.33-7.53 (2H, m), 7.76-7.84 (1H, m), 10.43 (1H, br s). LC-MS, m/z; 471 [M + H]+ |
| 238 | Et | H | F | —CO₂Et | ethyl 4-[3-(3- ethyl-7-fluoro- 1H-indazol-1- yl)-1,2,4- oxadiazol-5-yl]- 1,4′-bipiperidine-1′- carboxylate | ¹H-NMR (DMSO-d₆) δ: 1.16 (3H, t, J = 7.1 Hz), 1.34 (5H, t, J = 7.6 Hz), 1.69- 1.82 (4H, m), 2.08 (2H, d, J = 11.0 Hz), 2.28-2.34 (2H, m), 2.67-2.90 (4H, m), 3.03 (2H, q, J = 7.6 Hz), 3.06-3.17 (2H, m), 3.97-4.03 (4H, m), 7.34- 7.40 (1H, m), 7.41-7.48 (1H, m), 7.77 (1H, d, J = 7.3 Hz). LC-MS, m/z; 471 [M + H]+ |
| 239 | Et | F | H | 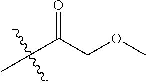 [SMILES: *C(=O)COC] | 1-{4-[3-(3- ethyl-6-fluoro- 1H-indazol-1- yl)-1,2,4- oxadiazol-5-yl]- 1,4′-bipiperidin-1′- yl}-2-methoxyethanone | ¹H-NMR (DMSO-d₆) δ: 1.22-1.43 (5H, m), 1.72- 1.84 (4H, m), 2.10 (2H, d, J = 10.7 Hz), 2.34 (2H, t, J = 10.5 Hz), 2.55 (2H, d, J = 12.4 Hz), 2.87-3.05 (5H, m), 3.09-3.15 (1H, m), 3.26 (3H, s), 3.78 (1H, d, J = 13.9 Hz), 4.05 (2H, dd, J = 29.4, 14.0 Hz), 4.35 (1H, d, J = 12.2 Hz), 7.25-7.31 (1H, m), 7.88-7.92 (1H, m), 7.98-8.03 (1H, m). LC-MS, m/z; 471 [M + H]+ |
| 240 | Et | H | Cl | —Ac | 1-{4-[3-(7- chloro-3-ethyl- 1H-indazol-1- yl)-1,2,4- oxadiazol-5-yl]- 1,4′-bipiperidin-1′- yl}ethanone hydrochloride | ¹H-NMR (DMSO-d₆) δ: 1.35 (3H, t, J = 7.6 Hz), 1.42-1.75 (2H, m), 1.95-2.20 (5H, m), 2.23-2.57 (5H, m), 2.95-3.23 (5H, m), 3.39-3.63 (4H, m), 3.89-4.02 (1H, m), 4.44-4.58 (1H, m), 7.39 (1H, t, J = 7.8 Hz), 7.64-7.71 (1H, m), 7.93-8.01 (1H, m), 10.90- 11.11 (1H, m). LC-MS, m/z; 457 [M + H]+ |
| 241 | Et | H | F | —Ms | 4-[3-(3-ethyl-7- fluoro-1H- indazol-1-yl)- 1,2,4-oxadiazol- 5-yl]-1′- (methylsulfonyl)- 1,4′-bipiperidine | ¹H-NMR (DMSO-d₆) δ: 1.34 (3H, t, J = 7.6 Hz), 1.49 (2H, m), 1.75-1.84 (4H, m), 2.05-2.13 (2H, m), 2.31- 2.43 (3H, m), 2.70 (2H, t, J = 11.1 Hz), 2.84 (3H, s), 2.89 (2H, d, J = 11.1 Hz), 3.03 (2H, q, J = 7.5 Hz), 3.08-3.17 (1H, m), 3.58 (2H, d, J = 12.0 Hz), 7.34-7.39 (1H, m), 7.42-7.48 (1H, m), 7.77 (1H, d, J = 7.7 Hz). LC-MS, m/z; 477 [M + H]+ |

### Example 242

### Preparation of 1-(4-{3-[7-fluoro-3-(propan-2-yl)-1H-indazol-1-yl]-1,2,4-oxadiazol-5-yl}-1,4′-bipiperidin-1′-yl)-2-hydroxyethanone

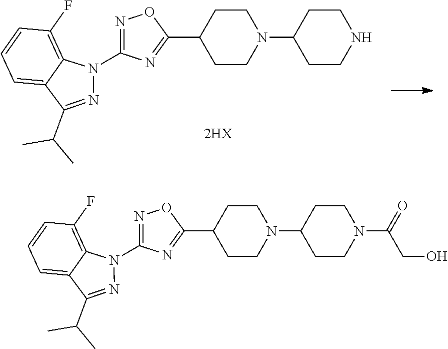 [SMILES: CC(C)c1nn(-c2noc(C3CCN(C4CCNCC4)CC3)n2)c2c(F)cccc12 ; CC(C)c1nn(-c2noc(C3CCN(C4CCN(C(=O)CO)CC4)CC3)n2)c2c(F)cccc12]

4-{3-[7-Fluoro-3-(propan-2-yl)-11′-indazol-1-yl]-1,2,4-oxadiazol-5-yl}-1,4′-bipiperidine dihydrochloride (130 mg) was dissolved in dichloromethane (4 ml). To the solution were added triethylamine (186 μl) and acetoxyacetyl chloride (43 μl), and the mixture was stirred at room temperature for 20 minutes. To the reaction solution was added saturated sodium bicarbonate (10 ml), and the mixture was extracted with ethyl acetate (20 ml). The organic layer was washed with water (10 ml), dried over sodium sulfate and filtered. The filtrate was concentrated under reduced pressure. The residue was suspended in methanol (2 ml), 2 N sodium hydroxide (15 μl) was added thereto, and the mixture was stirred at room temperature for 20 minutes. To the reaction mixture was added ethyl acetate (20 ml), and the mixture was washed with water (10 ml×2). The organic layer was dried over sodium sulfate and concentrated under reduced pressure, and the residue was purified by silica-gel chromatography (column; Hi-Flash™ Amino Column, developing solvent: ethyl acetate) to give the title compound (93 mg) as a white solid.

### Free Form

¹H-NMR (DMSO-d₆) δ: 1.24-1.48 (8H, m), 1.68-1.86 (4H, m), 2.04-2.16 (2H, m), 2.27-2.41 (2H, m), 2.49-2.66 (2H, m), 2.83-2.99 (3H, m), 3.08-3.20 (1H, m), 3.45-3.54 (1H, m), 3.65-3.76 (1H, m), 4.00-4.15 (2H, m), 4.32-4.42 (1H, m), 4.47 (1H, t, J=5.4 Hz), 7.33-7.50 (2H, m), 7.83 (1H, d, J=7.7 Hz).

HCl salt was obtained by treatment with 1N HCl/diethyl ether.

¹H-NMR (DMSO-d₆) δ: 1.41 (6H, d, J=6.8 Hz), 1.49-1.78 (2H, m), 2.02-2.69 (7H, m), 2.82-3.61 (8H, m), 3.68-3.94 (1H, m), 4.00-4.19 (2H, m), 4.36-4.78 (2H, m), 7.33-7.53 (2H, m), 7.79-7.88 (1H, m), 10.84-11.13 (1H, m).

LC-MS, m/z; 471 [M+H]+

### Preparations of Examples 243 to 244

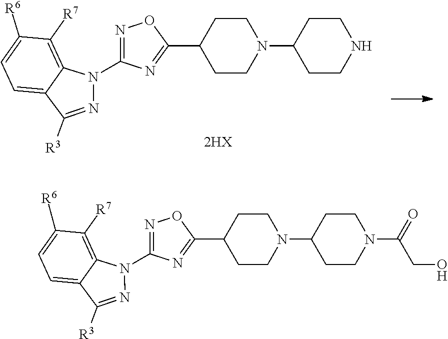

Wherein HX is hydrochloric acid or trifluoroacetic acid.

The compounds in the following table (i.e. Examples 243 to 244) were prepared in the same manner as in Example 242 except that the 4-{3-[7-fluoro-3-(propan-2-yl)-1H-indazol-1-yl]-1,2,4-oxadiazol-5-yl}-1,4′-bipiperidine dihydrochloride was replaced with the corresponding starting compound. Each of the hydrochlorides in the following table was obtained by dissolving the resultant compound in methylene chloride and then treating with 1N HCl/diethyl ether solution.

TABLE 37

| Ex. | R³ | R⁶ | R⁷ | Compound Name | ¹H-NMR/LC-MS, m/z |
|---|---|---|---|---|---|
| 243 | Et | F | H | 1-{4-[3-(3- | ¹H-NMR (DMSO-d₆) δ: |
| | | | | ethyl-6- | 1.34 (3H, t, J = 7.6 Hz), |
| | | | | fluoro-1H- | 1.50-1.72 (2H, |
| | | | | indazol-1- | m), 2.13 (2H, d, J = 11.0 Hz), |
| | | | | yl)-1,2,4- | 2.22-2.42 (4H, m), |
| | | | | oxadiazol- | 2.55-2.65 (1H, m), |
| | | | | 5-yl]-1,4′- | 2.99-3.06 (3H, m), |
| | | | | bipiperidin-1′-yl}- | 3.12-3.23 (2H, m), |
| | | | | 2- | 3.44-3.57 (4H, m), 3.86 (1H, d, |
| | | | | hydroxyethanone | J = 14.1 Hz), |
| | | | | hydrochloride | 4.04-4.17 (2H, m), |
| | | | | | 4.50 (1H, d, J = 13.4 Hz), |
| | | | | | 7.26-7.33 (1H, m), |
| | | | | | 7.87-7.93 (1H, m), |
| | | | | | 7.99-8.04 (1H, m), |
| | | | | | 10.73 (1H, s). |
| | | | | | LC-MS, m/z; |
| | | | | | 457 [M + H]+ |
| 244 | Et | H | F | 1-{4-[3-(3- | ¹H-NMR (DMSO-d₆) δ: |
| | | | | ethyl-7- | 1.42 (3H, t, J = 7.6 Hz), |
| | | | | fluoro-1H- | 1.56-1.80 (2H, |
| | | | | indazol-1- | m), 2.22 (2H, d, J = 11.0 Hz), |
| | | | | yl)-1,2,4- | 2.35-2.45 (4H, m), |
| | | | | oxadiazol- | 2.68 (1H, t, |
| | | | | 5-yl]-1,4′- | J = 12.2 Hz), |
| | | | | bipiperidin-1′-yl}- | 3.01-3.14 (3H, m), |
| | | | | 2- | 3.18-3.28 (2H, m), |
| | | | | hydroxyethanone | 3.50-3.64 (4H, m), |
| | | | | hydrochloride | 3.93 (1H, d, J = 12.2 Hz), |
| | | | | | 4.13-4.23 (2H, m), |
| | | | | | 4.57 (1H, d, J = 12.0 Hz), |
| | | | | | 7.42-7.49 (1H, |
| | | | | | m), 7.50-7.58 (1H, |
| | | | | | m), 7.85 (1H, d, J = 7.8 Hz), |
| | | | | | 11.15 (1H, |
| | | | | | s). |
| | | | | | LC-MS, m/z; |
| | | | | | 457 [M + H]+ |

### Preparations of Examples 245 to 246

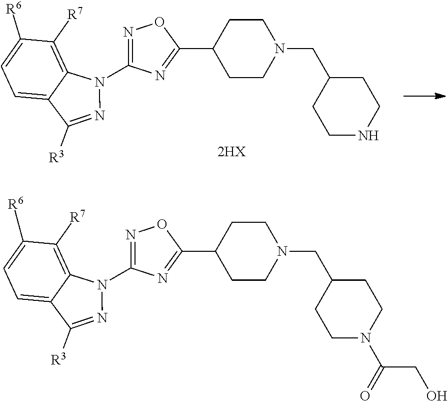

Wherein HX is hydrochloric acid or trifluoroacetic acid.

The compounds in the following table (i.e. Examples 245 to 246) were prepared in the same manner as in Example 242 except that the 4-{3-[7-fluoro-3-(propan-2-yl)-1H-indazol-1-yl]-1,2,4-oxadiazol-5-yl}-1,4′-bipiperidine dihydrochloride was replaced with the corresponding starting compound. The hydrochloride in the following table was obtained by dissolving the resultant compound in methylene chloride and then treating with 1N HCl/diethyl ether solution.

TABLE 38

| Ex. | R³ | R⁶ | R⁷ | Compound Name | ¹H-NMR/LC-MS, m/z |
|---|---|---|---|---|---|
| 245 | ⁱPr | H | F | 1-{4-[(4-{3-[7- | ¹H-NMR (CD₃OD) δ: |
| | | | | fluoro-3-(propan-2- | 1.17-1.39 (3H, m), |
| | | | | yl)-1H- | 1.48 (6H, d, J = 7.1 Hz), |
| | | | | indazol-1-yl]- | 1.80-3.22 (12H, m), |
| | | | | 1,2,4- | 3.48-3.62 (9H, m), |
| | | | | oxadiazol-5- | 7.29-7.40 (2H, m), |
| | | | | yl}piperidin-1- | 7.71-7.78 (1H, m). |
| | | | | yl)methyl]piperidin- | LC-MS, m/z; |
| | | | | 1-yl}-2- | 485 [M + H]+ |
| | | | | hydroxyethanone | |
| | | | | hydrochloride | |
| 246 | Et | H | F | 1-[4-({4-[3-(3-ethyl- | ¹H-NMR (DMSO-d₆) δ: |
| | | | | 7-fluoro-1H- | 0.89-1.04 (2H, m), |
| | | | | indazol- | 1.34 (3H, t, J = 7.5 Hz), |
| | | | | 1-yl)-1,2,4- | 1.68-1.83 (5H, m), |
| | | | | oxadiazol-5- | 2.06-2.15 (6H, m), |
| | | | | yl]piperidin-1- | 2.58-2.66 (1H, m), |
| | | | | yl}methyl)piperidin- | 2.81-2.95 (3H, m), 3.03 (2H, |
| | | | | 1-yl]-2- | q, J = 7.6 Hz), |
| | | | | hydroxyethanone | 3.10-3.18 (1H, m), |
| | | | | | 3.62 (1H, d, J = 11.1 Hz), |
| | | | | | 4.04 (2H, |
| | | | | | t, J = 5.4 Hz), |
| | | | | | 4.32 (1H, d, J = 11.1 Hz), |
| | | | | | 4.41 (1H, |
| | | | | | t, J = 5.4 Hz), |
| | | | | | 7.34-7.40 (1H, m), |
| | | | | | 7.42-7.48 (1H, m), |
| | | | | | 7.75-7.79 (1H, m). |
| | | | | | LC-MS, m/z; |
| | | | | | 471 [M + H]+ |

### Example 247

### Preparation of 1-[(3R)-3-({4-[3-(3-ethyl-7-fluoro-1H-indazol-1-yl)-1,2,4-oxadiazol-5-yl]piperidin-1-yl}methyl)pyrrolidin-1-yl]-2-hydroxyethanone

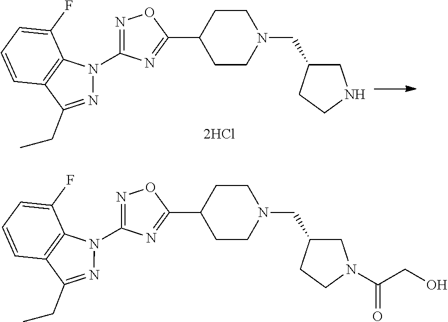 [SMILES: CCc1nn(-c2noc(C3CCN(C[C@H]4CCNC4)CC3)n2)c2c(F)cccc12 ; CCc1nn(-c2noc(C3CCN(C[C@H]4CCN(C(=O)CO)C4)CC3)n2)c2c(F)cccc12]

The title compound was prepared in the same manner as in Example 242 except that the 4-[(3-[7-fluoro-3-(propan-2-yl)-1H-indazol-1-yl]-1,2,4-oxadiazol-5-yl]-1,4′-bipiperidine dihydrochloride was replaced with 3-ethyl-7-fluoro-1-(5-{1-[(3S)-pyrrolidin-3-ylmethyl]piperidin-4-yl}-1,2,4-oxadiazol-3-yl)-1H-indazole dihydrochloride.

¹H-NMR (CDCl₃) δ: 1.44 (3H, t, J=7.6 Hz), 1.56-1.90 (2H, m), 1.96-2.29 (7H, m), 2.29-2.62 (3H, m), 2.84-3.17 (5H, m), 3.18-3.61 (3H, m), 3.73 (1H, m), 4.09 (2H, d, J=3.7 Hz), 7.20-7.33 (2H, m), 7.54 (1H, m).

LC-MS, m/z; 457 [M+H]+

### Example 248

### Preparation of 1-{(3R)-3-[(4-{3-[7-fluoro-3-(propan-1) 1H-indazol-1-yl]-1,2,4-oxadiazol-5-yl}piperidin-1-yl)methyl]pyrrolidin-1-yl}-2-hydroxyethanone hydrochloride

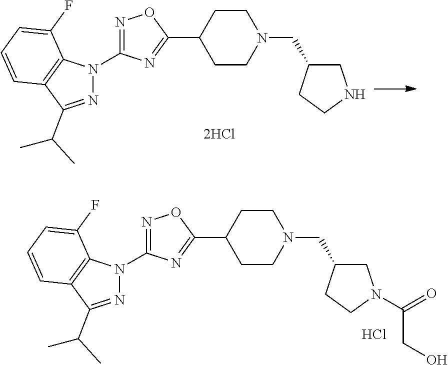 [SMILES: CC(C)c1nn(-c2noc(C3CCN(C[C@H]4CCNC4)CC3)n2)c2c(F)cccc12 ; CC(C)c1nn(-c2noc(C3CCN(C[C@H]4CCN(C(=O)CO)C4)CC3)n2)c2c(F)cccc12 ; Cl]

An intermediate was prepared in the same manner as in Example 242 except that the 4-{3-[7-fluoro-3-(propan-2-yl)-1H-indazol-1-yl]-1,2,4-oxadiazol-5-yl}-1,4′-bipiperidine dihydrochloride was replaced with 7-fluoro-3-(propan-2-yl)-1-(5-{1-[(3S)-pyrrolidin-3-ylmethyl]piperidin-4-yl}-1,2,4-oxadiazol-3-yl)-1H-indazole dihydrochloride, and then the intermediate was dissolved in methylene chloride and treated with 1N HCl/diethyl ether solution to give the title compound of hydrochloride.

¹H-NMR (CD₃OD) δ: 1.48 (6H, d, J=7.0 Hz), 1.62-2.90 (3H, m), 3.08-4.17 (19H, m), 7.32-7.36 (2H, m), 7.72-7.75 (1H, m).

LC-MS, m/z; 471[M+H]+

### Preparations of Examples 249 to 250

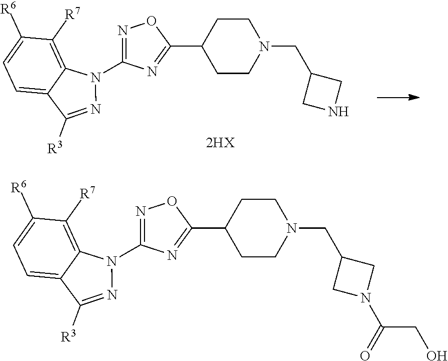

Wherein HX is hydrochloric acid or trifluoroacetic acid.

The compounds in the following table (i.e. Examples 249 to 250) were prepared in the same manner as in Example 242 except that the 4-{3-[7-fluoro-3-(propan-2-yl)-1H-indazol-1-yl]-1,2,4-oxadiazol-5-yl}-1,4′-bipiperidine dihydrochloride was replaced with the corresponding starting compound. The hydrochloride in the following table was obtained by dissolving the resultant compound in methylene chloride and then treating with 1N HCl/diethyl ether solution.

TABLE 39

| Ex. | R³ | R⁶ | R⁷ | Compound Name | ¹H-NMR/LC-MS, m/z |
|---|---|---|---|---|---|
| 249 | ⁱPr | H | F | 1-{3-[(4-{3-[7- | ¹H-NMR (CD₃OD) δ: |
| | | | | fluoro-3-(propan- | 1.47 (6H, d, J = 7.0 Hz), |
| | | | | 2-yl)-1H-indazol- | 1.73-3.13 (4H, m), |
| | | | | 1-yl]-1,2,4- | 3.24-4.59 (16H m), |
| | | | | oxadiazol-5- | 7.32-7.36 (2H, m), |
| | | | | yl}piperidin-1- | 7.72-7.75 (1H, m). |
| | | | | yl)methyl]azetidin | LC-MS, m/z; 457 [M + H]+ |
| | | | | -1-yl}-2- | |
| | | | | hydroxyethanone | |
| | | | | hydrochloride | |
| 250 | Et | H | F | 1-[3-({4-[3- | ¹H-NMR (DMSO-d₆) δ: |
| | | | | (3-ethyl- | 1.34 (3H, t, J = 7.6 Hz), |
| | | | | 7-fluoro-1H- | 1.78 (2H, q, J = 11.0 Hz), |
| | | | | indazol- | 2.04-2.17 (4H, m), 2.53 (2H, |
| | | | | 1-yl)-1,2,4- | d, J = 6.3 Hz), |
| | | | | oxadiazol-5- | 2.81 (3H, br s), 3.02 (2H, |
| | | | | yl]piperidin-1- | q, J = 7.6 Hz), |
| | | | | yl}methyl)azetidin- | 3.13-3.13 (1H, m), |
| | | | | 1-yl]-2- | 3.49 (1H, dd, J = 9.5, |
| | | | | hydroxyethanone | 5.6 Hz), 3.78 (1H, dd, |
| | | | | | J = 9.0, 5.6 Hz), |
| | | | | | 3.86 (2H, d, J = 6.1 Hz), |
| | | | | | 3.93 (1H, t, J = 9.0 Hz), |
| | | | | | 4.20 (1H, t, J = 8.5 Hz), |
| | | | | | 4.83 (1H, t, |
| | | | | | J = 6.1 Hz), |
| | | | | | 7.33-7.39 (1H, m), |
| | | | | | 7.42-7.47 (1H, m), |
| | | | | | 7.77 (1H, d, J = 8.0 Hz). |
| | | | | | LC-MS, m/z; |
| | | | | | 443 [M + H]+ |

### Example 251

### Preparation of 1′-ethyl-4-[3-(3-ethyl-7-fluoro-1H-indazol-1-yl)-1,2,4-oxadiazol-5-yl]-1,4′-bipiperidine dihydrochloride

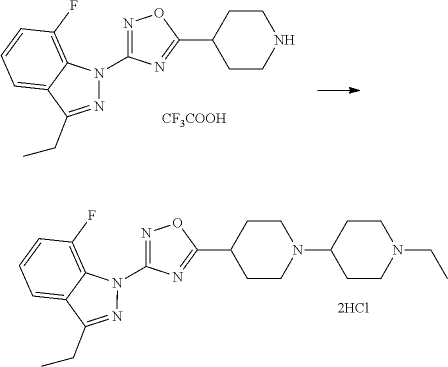 [SMILES: CCc1nn(-c2noc(C3CCNCC3)n2)c2c(F)cccc12 ; "CF3COOH" ; CCc1nn(-c2noc(C3CCN(C4CCN(CC)CC4)CC3)n2)c2c(F)cccc12]

An intermediate was prepared in the same manner as in Example 134 except that the 3-ethyl-6-fluoro-1-[5-(piperidin-4-yl)-1,2,4-oxadiazol-3-yl]-1H-indazole trifluoroacetate and tetrahydropyrane-4-carboaldehyde were replaced with the 3-ethyl-7-fluoro-1-[5-(piperidin-4-yl)-1,2,4-oxadiazol-3-yl]-1H-indazole trifluoroacetate and 1-ethyl-4-piperidinone, respectively, and then the intermediate was dissolved in methylene chloride and then treated with 1N HCl/diethyl ether to give the title compound (60 mg) as a white solid.

LC-MS, m/z; 427 [M+H]+

### Example 252

### Preparation of (4-{3-[7-fluoro-3-(propan-2-yl)-1H-indazol-1-yl]-1,2,4-oxadiazol-5-yl}-1,4′-bipiperidin-1′-yl)(oxetan-3-yl)methanone

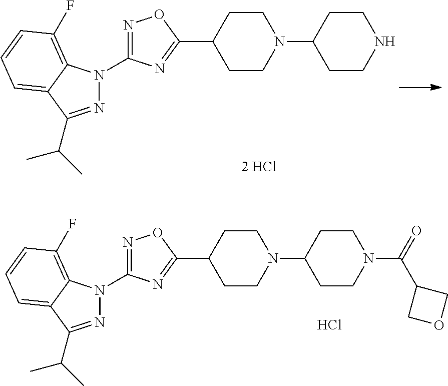 [SMILES: CC(C)c1nn(-c2noc(C3CCN(C4CCNCC4)CC3)n2)c2c(F)cccc12 ; "2 HCl" ; CC(C)c1nn(-c2noc(C3CCN(C4CCN(C(=O)C5COC5)CC4)CC3)n2)c2c(F)cccc12 ; Cl]

4-{3-[7-Fluoro-3-(propan-2-yl)-1H-indazol-1-yl]-1,2,4-oxadiazol-5-yl}-1,4′-bipiperidine dihydrochloride (120 mg) was dissolved in dimethylformamide (4 ml). To the solution were added triethylamine (276 μl), 3-oxetanecarboxylic acid (56 mg), 1-ethyl-3-(3-dimethylaminopropyl)carbodiimide hydrochloride (114 mg) and 1-hydroxybenzotriazole (34 mg), and the mixture was stirred at room temperature overnight. To the reaction solution was added ethyl acetate (20 ml), and the mixture was washed with water (10 ml×2). The organic layer was dried over sodium sulfate and concentrated under reduced pressure. The residue was purified by silica-gel chromatography (column; Hi-Flash™ Amino Column, developing solvent: ethyl acetate) to give the title compound (15 mg) as a colorless oil.

LC-MS, m/z; 497 [M+H]+

### Example 253

### Preparation of 2,2-difluoro-1-(4-{3-[7-fluoro-3-(propan-2-yl)-1H-indazol-1-yl]-1,2,4-oxadiazol-5-yl}-1,4′-bipiperidin-1′-yl)ethanone

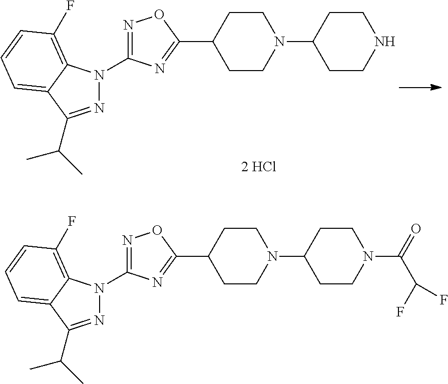 [SMILES: CC(C)c1nn(-c2noc(C3CCN(C4CCNCC4)CC3)n2)c2c(F)cccc12 ; "2 HCl" ; CC(C)c1nn(-c2noc(C3CCN(C4CCN(C(=O)C(F)F)CC4)CC3)n2)c2c(F)cccc12]

4-{3-[7-Fluoro-3-(propan-2-yl)-1H-indazol-1-yl]-1,2,4-oxadiazol-5-yl}-1,4′-bipiperidine dihydrochloride (120 mg) was dissolved in dimethylformamide (4 ml). To the solution were added triethylamine (276 μl), 2,2-difluoroacetic acid (52 mg), 1-ethyl-3-(3-dimethylaminopropyl)carbodiimide hydrochloride (114 mg) and 1-hydroxybenzotriazole (34 mg), and the mixture was stirred at room temperature overnight. To the reaction solution was added ethyl acetate (20 ml), and the mixture was washed with water (10 ml×2). The organic layer was dried over sodium sulfate and concentrated under reduced pressure. The residue was purified by silica-gel chromatography (column; Hi-Flash™ Amino Column, developing solvent: ethyl acetate) to give the title compound (72 mg) as a colorless oil.

¹H-NMR (CDCl₃) δ: 1.44-1.69 (9H, m), 1.85-2.11 (4H, m), 2.13-2.25 (2H, m), 2.32-2.47 (2H, m), 2.55-2.81 (2H, m), 2.91-3.16 (4H, m), 3.39-3.56 (1H, m), 4.07-4.22 (1H, m), 4.48-4.61 (1H, m), 7.17-7.29 (2H, m), 7.54-7.63 (1H, m).

LC-MS, m/z; 491 [M+H]+

### Example 254

### Preparation of 4-{3-[7-fluoro-3-(propan-2-yl)-1,4-indazol-1-yl]-1,2,4-oxadiazol-5-yl}-N-methyl-1,4′-bipiperidine-1′-carboxamide

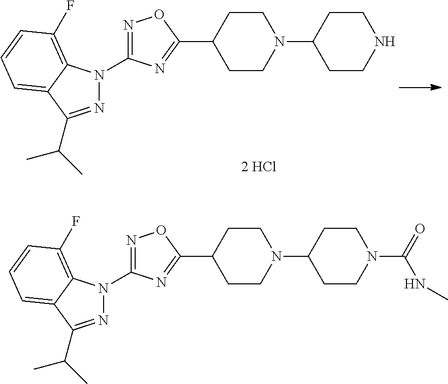 [SMILES: CC(C)c1nn(-c2noc(C3CCN(C4CCNCC4)CC3)n2)c2c(F)cccc12 ; "2 HCl" ; CNC(=O)N1CCC(N2CCC(c3nc(-n4nc(C(C)C)c5cccc(F)c54)no3)CC2)CC1]

2.0 M Methylamine/THF (247 μl) and carbodiimidazole (88 mg) were dissolved in THF (1.0 ml), and the solution was stirred at room temperature for 1 hour. To the reaction solution were added dropwise 4-{3-[7-fluoro-3-(propan-2-yl)-1H-indazol-1-yl]-1,2,4-oxadiazol-5-yl}-1,4′-bipiperidine dihydrochloride (120 mg) and triethylamine (103 μl) in THF (2 ml), and the mixture was stirred at room temperature overnight. To the reaction solution was added saturated sodium bicarbonate aqueous solution (10 ml), and the mixture was extracted with ethyl acetate (20 ml). The organic layer was washed with brine, dried over sodium sulfate, and concentrated under reduced pressure. The residue was purified by silica-gel chromatography (column; Hi-Flash™ Amino Column, developing solvent: ethyl acetate) to give the title compound (104 mg) as a colorless oil.

¹H-NMR (CDCl₃) δ: 1.37-1.59 (8H, m), 1.76-1.90 (2H, m), 1.92-2.59 (7H, m), 2.67-2.86 (5H, m), 2.93-3.12 (3H, m), 3.40-3.53 (1H, m), 3.91-4.07 (2H, m), 4.44-4.58 (1H, m), 7.17-7.30 (2H, m), 7.54-7.63 (1H, m).

LC-MS, m/z; 470 [M+H]+

### Example 255

### Preparation of (2R)-2-({4-[3-(3-ethyl-7-fluoro-1H-indazol-1-yl)-1,2,4-oxadiazol-5-yl]piperidin-1-yl}methyl)-N-methylpyrrolidine-1-carboxamide

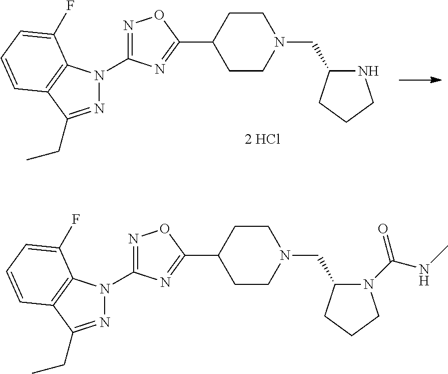 [SMILES: CCc1nn(-c2noc(C3CCN(C[C@H]4CCCN4)CC3)n2)c2c(F)cccc12 ; "2 HCl" ; CCc1nn(-c2noc(C3CCN(C[C@H]4CCCN4C(=O)NC)CC3)n2)c2c(F)cccc12]

The title compound was prepared in the same manner as in Example 254 except that the 4-{3-[7-fluoro-3-(propan-2-yl)-1H-indazol-1-yl]-1,2,4-oxadiazol-5-yl}-1,4′-bipiperidine dihydrochloride was replaced with 3-ethyl-7-fluoro-1-(5-{1-[(2R)-pyrrolidin-2-ylmethyl]piperidin-4-yl}-1,2,4-oxadiazol-3-yl)-1H-indazole dihydrochloride.

¹H-NMR (CDCl₃) δ: 1.44 (3H, t, J=7.6 Hz), 1.59 (1H, m), 1.77 (2H, m), 1.92-2.11 (3H, m), 2.15-2.31 (3H, m), 2.32-2.45 (2H, m), 2.54 (1H, dd, J=13.4, 8.6 Hz), 2.78 (3H, d, J=3.7 Hz), 2.97 (1H, m), 3.02-3.20 (4H, m), 3.28 (1H, m), 3.66-3.86 (2H, m), 7.20-7.33 (2H, m), 7.54 (1H, s), 7.77 (1H, bs).

LC-MS, m/z; 456 [M+H]+

### Example 256

### Preparation of (3R)-3-({4-[3-(3-ethyl-7-fluoro-1H-indazol-1-yl)-1,2,4-oxadiazol-5-yl]piperidin-1-yl}methyl)-N-methylpyrrolidine-1-carboxamide

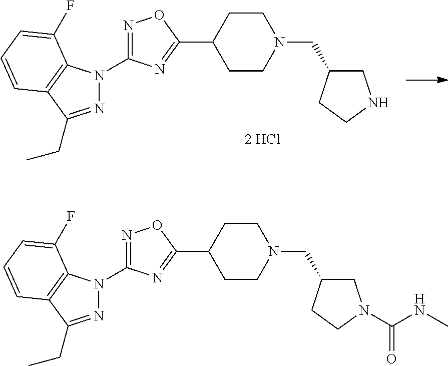 [SMILES: CCc1nn(-c2noc(C3CCN(C[C@H]4CCNC4)CC3)n2)c2c(F)cccc12 ; "2 HCl" ; CCc1nn(-c2noc(C3CCN(C[C@H]4CCN(C(=O)NC)C4)CC3)n2)c2c(F)cccc12]

The title compound was prepared in the same manner as in Example 254 except that the 4-{3-[7-fluoro-3-(propan-2-yl)-1H-indazol-1-yl]-1,2,4-oxadiazol-5-yl}-1,4′-bipiperidine dihydrochloride was replaced with 3-ethyl-7-fluoro-1-(5-{1-[(3S)-pyrrolidin-3-ylmethyl]piperidin-4-yl}-1,2,4-oxadiazol-3-yl)-1H-indazole dihydrochloride.

LC-MS, m/z; 456 [M+H]+

### Example 257

### Preparation of methyl 4-{3-[3-(3-ethyl-1H-indazol-1-yl)-1,2,4-oxadiazol-5-yl]azetidin-1-yl}piperidine-1-carboxylate

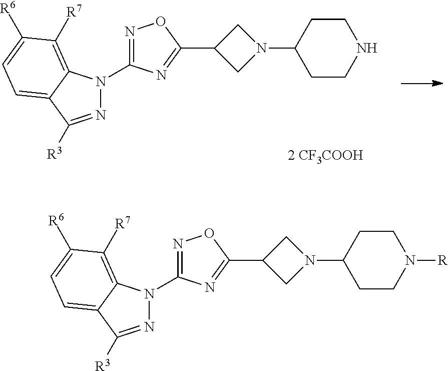

The compound in the following table (Example 257) was prepared in the same manner as in Example 168 except that the 3-ethyl-1-(5-{1-[2-(piperidin-4-yl)ethyl]piperidin-4-yl}-1,2,4-oxadiazol-3-yl)-1H-indazole bis(trifluoroacetate) was replaced with 3-ethyl-1-{5-[1-(piperidin-4-yl)azetidin-3-yl]-1,2,4-oxadiazol-3-yl}-1H-indazole bis(trifluoroacetate). The free form of the compound in the following table was obtained by omitting the conversion step into hydrochloride in Example 168.

TABLE 40

| | | | | | | ¹H NMR/ |
|---|---|---|---|---|---|---|
| Ex. | R³ | R⁶ | R⁷ | R | Compound Name | LC-MS, m/z |
| 257 | Et | H | H | —CO₂Me | methyl 4-{3-[3-(3- | LC-MS, m/z; |
| | | | | | ethyl-1H-indazol-1- | 411 [M + H]+ |
| | | | | | yl)-1,2,4-oxadiazol-5- | |
| | | | | | yl]azetidin-1- | |
| | | | | | yl}piperidine-1- | |
| | | | | | carboxylate | |

### Example 258

### Preparation of 1-(4-{3-[3-(3-ethyl-6-fluoro-1H-indazol-1-yl)-1,2,4-oxadiazol-5-yl]azetidin-1-yl}piperidin-1-yl)ethanone trifluoroacetate

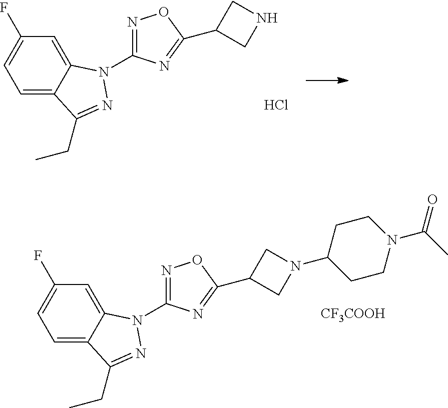

1-[5-(Azetidin-3-yl)-1,2,4-oxadiazol-3-yl]-3-ethyl-6-fluoro-1H-indazole hydrochloride (100 mg) was dissolved in methanol (10 ml). To the solution were added 1-acetylpiperidin-4-one (56 mg), acetic acid (24 mg) and sodium cyanoborohydride (41 mg), and the mixture was stirred at room temperature overnight. The reaction solution was filtered, the filtrate was concentrated, water was added thereto, and the mixture was extracted with dichloromethane. The organic layer was dried over sodium sulfate and concentrated under reduced pressure. The residue was purified by reverse phase HPLC to give the title compound (29 mg) as a white solid.

LC-MS, m/z; 413 [M+H]+

### Example 259

### Preparation of methyl 4-{3-[3-(3-ethyl-6-fluoro-1H-indazol-1-yl)-1,2,4-oxadiazol-5-yl]azetidin-1-yl}piperidine-1-carboxylate trifluoroacetate

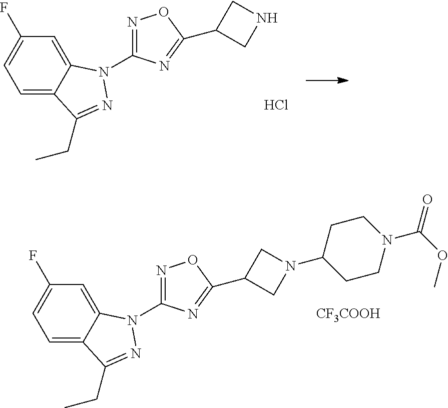

The title compound was prepared in the same manner as in Example 258 except that 1-acetylpiperidin-4-one was replaced with methyl 4-oxopiperidine-1-carboxylate.

LC-MS, m/z; 429 [M+H]+

### Example 260

### Preparation of methyl 3-{3-[3-(3-ethyl-6-fluoro-1H-indazol-1-yl)-1,2,4-oxadiazol-5-yl]azetidin-1-yl}pyrrolidine-1-carboxylate trifluoroacetate

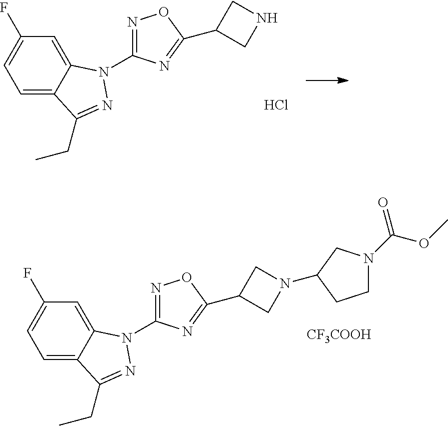

The title compound was prepared in the same manner as in Example 258 except that the 1-acetylpiperidin-4-one was replaced with methyl 3-oxopyrrolidine-1-carboxylate.

LC-MS, m/z; 415 [M+H]+

### Example 261

### Preparation of 3-{4-[3-(3-ethyl-1H-indazol-1-yl)-1,2,4-oxadiazol-5-yl]piperidin-1-yl}propan-1-ol

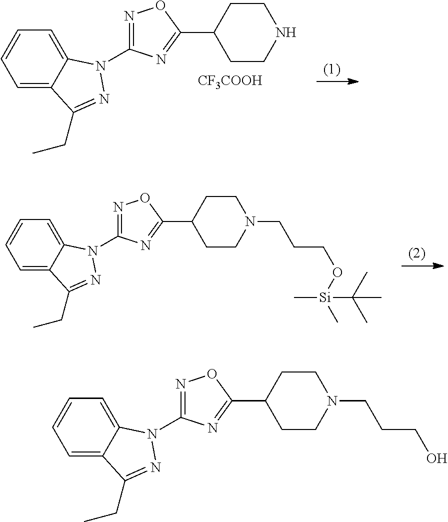

(1) 3-Ethyl-1-[5-(piperidin-4-yl)-1,2,4-oxadiazol-3-yl]-1H-indazole trifluoroacetate (120 mg) was suspended in N,N-dimethylformamide (3 ml). To the suspension were added (3-bromopropoxy)(tert-butyl)dimethylsilane (103 mg) and potassium carbonate (161 mg), and the mixture was refluxed overnight. The reaction solution was cooled to room temperature and water was added thereto. The mixture was extracted with ethyl acetate. The organic layer was washed with water and brine, dried over sodium sulfate, and filtered. The filtrate was concentrated under reduced pressure, and the residue was purified by silica-gel chromatography (column; Hi-Flash™ Amino column, developing solvent: hexane/ethyl acetate=2:1) to give 1-{5-[1-(3-{[tert-butyl(dimethyl)silyl]oxy}propyl)piperidin-4-yl]-1,2,4-oxadiazol-3-yl}-3-ethyl-1H-indazole (103 mg) as a white solid.

LC-MS, m/z; 470 [M+H]+

(2) 1-{5-[1-(3-{[Tert-butyl(dimethyl)silyl]oxy}propyl)piperidin-4-yl]-1,2,4-oxadiazol-3-yl}-3-ethyl-1H-indazole (100 mg) was dissolved in dichloromethane (5 ml). To the solution was added 1N tetrabutylammonium fluoride in tetrahydrofuran (0.3 ml), and the mixture was stirred at 70° C. for 4 hours. The reaction solution was cooled to room temperature, water was added to the reaction solution, and the mixture was extracted with ethyl acetate. The organic layer was washed with brine, dried over sodium sulfate and filtered, and the filtrate was concentrated under reduced pressure. The residue was purified by silica-gel chromatography (column; Hi-Flash™ Amino Column, developing solvent: chloroform/methanol) to give the title compound (58 mg) as a colorless oil.

LC-MS, m/z; 356 [M+H]+

### Example 262

### Preparation of 7-fluoro-1-{5-[1-(4-methoxycyclohexyl)piperidin-4-yl]-1,2,4-oxadiazol-3-yl}-3-(propan-2-yl)-1H-indazole

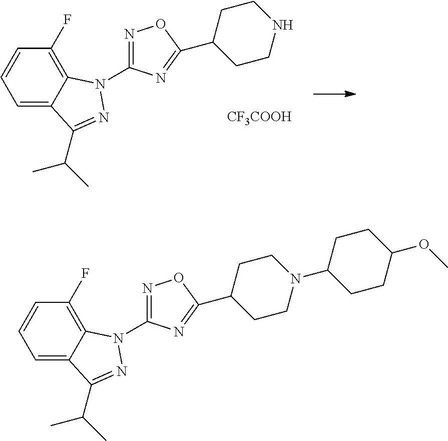 [SMILES: CC(C)c1nn(-c2noc(C3CCNCC3)n2)c2c(F)cccc12 ; "CF3COOH" ; COC1CCC(N2CCC(c3nc(-n4nc(C(C)C)c5cccc(F)c54)no3)CC2)CC1]

7-Fluoro-1-[5-(piperidin-4-yl)-1,2,4-oxadiazol-3-yl]-3-(propan-2-yl)-1H-indazole trifluoroacetate (250 mg) was dissolved in dichloromethane (5 ml). To the solution were added 4-methoxycyclohexanone (145 mg) and sodium triacetoxyborohydride (358 mg), and the mixture was stirred at room temperature for 2 days. To the reaction solution was added saturated sodium bicarbonate (10 ml), and the mixture was extracted with ethyl acetate (20 ml). The organic layer was washed with water (10 ml×2), dried over sodium sulfate, and concentrated under reduced pressure. The residue was purified by silica-gel chromatography (column; Hi-Flash™ Amino Column, developing solvent: hexane/ethyl acetate=2:1) to give the title compound (184 mg) as a colorless oil.

¹H-NMR (CDCl₃) δ: 1.14-1.72 (11H, m), 1.76-2.61 (10H, m), 2.75-3.61 (8H, m), 7.15-7.30 (2H, m), 7.51-7.64 (1H, m).

LC-MS, m/z; 442 [M+H]+

### Example 263

Preparations of (1S,2S) and (1R,2R)-2-(4-{3-[7-fluoro-3-(propan-2-yl)-1H-indazol-1-yl]-1,2,4-oxadiazol-5-yl}piperidin-1-yl)cyclohexanol

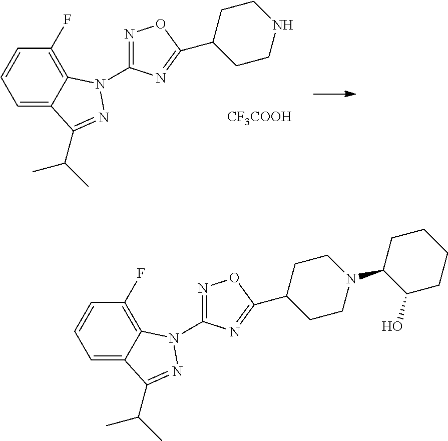 [SMILES: CC(C)c1nn(-c2noc(C3CCNCC3)n2)c2c(F)cccc12 ; "CF3COOH" ; CC(C)c1nn(-c2noc(C3CCN([C@H]4CCCC[C@@H]4O)CC3)n2)c2c(F)cccc12]

To a mixture of 7-fluoro-1-[5-(piperidin-4-yl)-1,2,4-oxadiazol-3-yl]-3-(propan-2-yl)-1H-indazole trifluoroacetate (120 mg) and ethanol (2.0 ml) were added N,N-diisopropylethylamine (174 μl) and 7-oxabicyclo[4.1.0]heptane (133 mg), and the mixture was stirred under reflux for 2 days. The reaction solution was concentrated and the residue was purified by silica-gel chromatography (column; Hi-Flash™ Amino Column, developing solvent: hexane/ethyl acetate=1:1) to give the title compound (106 mg) as a colorless oil.

¹H-NMR (CDCl₃) δ: 1.09-1.36 (4H, m), 1.42-1.58 (7H, m), 1.65-1.86 (3H, m), 1.90-2.35 (7H, m), 2.70-2.83 (2H, m), 2.96-3.11 (2H, m), 3.37-3.56 (2H, m), 7.18-7.30 (2H, m), 7.55-7.63 (1H, m). LC-MS, m/z; 428 [M+H]+

### Example 264

Preparations of (1S,2S) and (1R,2R)-2-(4-{3-[7-fluoro-3-(propan-2-yl)-1H-indazol-1-yl]-1,2,4-oxadiazol-5-yl}piperidin-1-yl)cyclopentanol

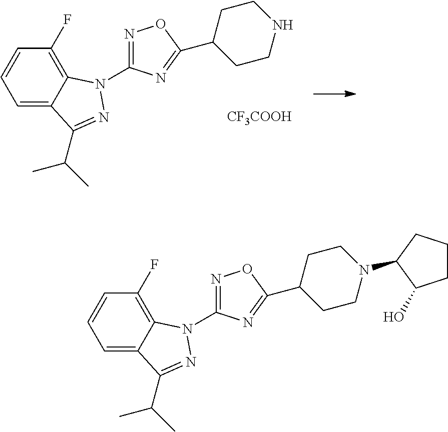 [SMILES: CC(C)c1nn(-c2noc(C3CCNCC3)n2)c2c(F)cccc12 ; "CF3COOH" ; CC(C)c1nn(-c2noc(C3CCN([C@H]4CCC[C@@H]4O)CC3)n2)c2c(F)cccc12]

The title compound was prepared in the same manner as in Example 263 except that the 7-oxabicyclo[4.1.0]heptane was replaced with 6-oxabicyclo[3.1.0]hexane.

¹H-NMR (CDCl₃) δ: 1.50 (6H, d, J=7.1 Hz), 1.53-1.75 (4H, m), 1.84-2.39 (9H, m), 2.53-2.63 (1H, m), 2.98-3.22 (3H, m), 3.43-3.54 (1H, m), 4.14 (1H, dd, J=13.0, 5.7 Hz), 7.18-7.29 (2H, m), 7.54-7.62 (1H, m).

LC-MS, m/z; 414 [M+H]+

### Example 265

### Preparation of N-(2-{4-[3-(3-methyl-1H-indazol-1-yl)-1,2,4-oxadiazol-5-yl]piperidin-1-yl}ethyl)benzamide

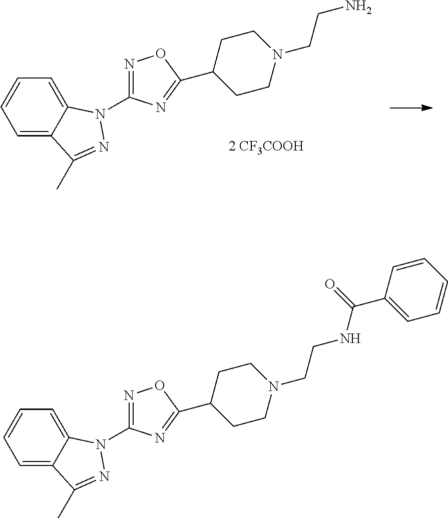 [SMILES: Cc1nn(-c2noc(C3CCN(CCN)CC3)n2)c2ccccc12 ; "2 CF3COOH" ; Cc1nn(-c2noc(C3CCN(CCNC(=O)c4ccccc4)CC3)n2)c2ccccc12]

The title compound (13 mg) as a white solid was prepared in the same manner as in Example 168 except that the 3-ethyl-1-(5-{1-[2-(piperidin-4-yl)ethyl]piperidin-4-yl}-1,2,4-oxadiazol-3-yl)-1H-indazole bis(trifluoroacetate) and methyl chloroformate were replaced with 2-{4-[3-(3-methyl-1H-indazol-1-yl)-1,2,4-oxadiazol-5-yl]piperidin-1-yl}ethanamine bis(trifluoroacetate) and benzoyl chloride, respectively, and the conversion step into hydrochloride was omitted.

¹H-NMR (DMSO-d₆) δ: 1.81-1.90 (2H, m), 2.11 (2H, d, J=10.7 Hz), 2.20 (2H, t, J=10.7 Hz), 2.51-2.54 (2H, m), 2.60 (3H, s), 2.95 (2H, d, J=11.8 Hz), 3.10-3.17 (1H, m), 3.40 (2H, q, J=6.6 Hz), 7.37-7.53 (4H, m), 7.65 (1H, m), 7.83 (2H, d, J=6.8 Hz), 7.91 (1H, d, J=7.8 Hz), 8.20 (1H, d, J=8.5 Hz), 8.41 (1H, t, J=5.6 Hz).

LC-MS, m/z; 431 [M+H]+

### Preparations of Examples 266 to 268

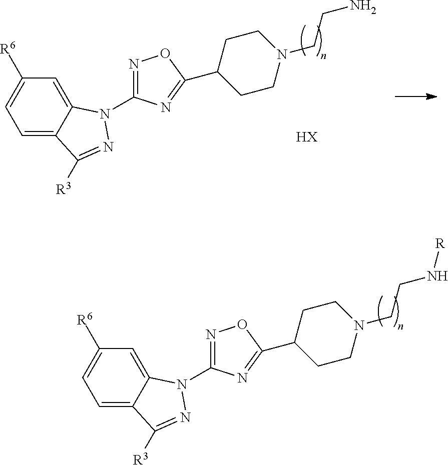

Wherein HX is hydrochloric acid or trifluoroacetic acid.

The compounds in the following table (i.e. Examples 266 to 268) were prepared in the same manner as in Example 265 except that the 2-{4-[3-(3-methyl-1H-indazol-1-yl)-1,2,4-oxadiazol-5-yl]piperidin-1-yl}ethanamine bis(trifluoroacetate) and benzoyl chloride were replaced with the corresponding starting compound and acid chloride (defined as R—Cl), respectively.

TABLE 41

| Ex. | R³ | R⁶ | n | R | Compound Name | ¹H-NMR/LC-MS, m/z |
|---|---|---|---|---|---|---|
| 266 | Me | H | 1 | —Ms | N-(2-{4-[3-(3- | ¹H-NMR (DMSO-d₆) δ: |
| | | | | | methyl-1H- | 1.80-1.90 (2H, m), |
| | | | | | indazol-1-yl)- | 2.08-2.20 (4H, m), |
| | | | | | 1,2,4-oxadiazol- | 2.44 (2H, t, J = 6.7 Hz), |
| | | | | | 5-yl]piperidin-1- | 2.60 (3H, |
| | | | | | yl}ethyl)methanesulfonamide | s), 2.87-2.93 (5H, |
| | | | | | | m), 3.05-3.17 (3H, |
| | | | | | | m), 6.88 (1H, s), |
| | | | | | | 7.39 (1H, m), |
| | | | | | | 7.65 (1H, m), 7.91 (1H, |
| | | | | | | d, J = 7.8 Hz), |
| | | | | | | 8.20 (1H, d, J = 8.5 Hz). |
| | | | | | | LC-MS, |
| | | | | | | m/z; 405 [M + H]+ |
| 267 | Me | H | 2 | —CO₂Me | methyl (3-{4-[3- | LC-MS, m/z; |
| | | | | | (3-methyl-1H- | 399 [M + H]+ |
| | | | | | indazol-1-yl)- | |
| | | | | | 1,2,4-oxadiazol- | |
| | | | | | 5-yl]piperidin-1- | |
| | | | | | yl}propyl)carbamate | |
| 268 | Et | F | 1 | —CO₂Me | methyl (2-{4-[3- | ¹H-NMR (CDCl₃) δ: |
| | | | | | (3-ethyl-6- | 1.44 (3H, t, J = 7.6 Hz), |
| | | | | | fluoro-1H- | 1.97-2.27 (6H, m), 2.52 (2H, |
| | | | | | indazol-1-yl)- | t, J = 5.9 Hz), |
| | | | | | 1,2,4-oxadiazol- | 2.89-3.13 (5H, m), |
| | | | | | 5-yl]piperidin-1- | 3.24-3.39 (2H, m), |
| | | | | | yl}ethyl)carbamate | 3.69 (3H, s), |
| | | | | | | 5.05-5.29 (1H, m), |
| | | | | | | 7.08 (1H, td, J = 8.9, |
| | | | | | | 2.2 Hz), |
| | | | | | | 7.70 (1H, dd, J = 8.7, |
| | | | | | | 5.0 Hz), 7.98 (1H, |
| | | | | | | dd, J = 9.3, 2.2 Hz). |
| | | | | | | LC-MS, m/z; |
| | | | | | | 417 [M + H]+ |

### Example 269

### Preparation of methyl methyl(3-{4-[3-(3-methyl-1H-indazol-1-yl)-1,2,4-oxadiazol-5-yl]piperidin-1-yl}propyl)carbamate

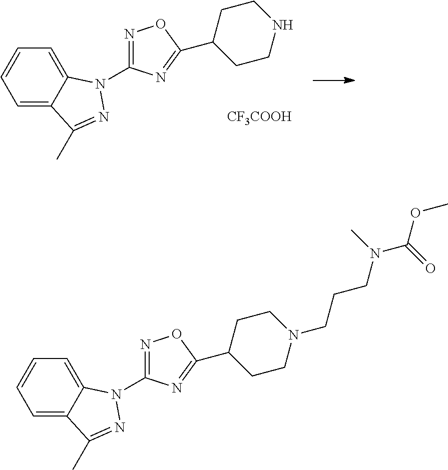 [SMILES: Cc1nn(-c2noc(C3CCNCC3)n2)c2ccccc12 ; "CF3COOH" ; COC(=O)N(C)CCCN1CCC(c2nc(-n3nc(C)c4ccccc43)no2)CC1]

The title compound (57 mg) as a white solid was prepared in the same manner as in Example 097 except that the 3-ethyl-6-fluoro-1-[5-(piperidin-4-yl)-1,2,4-oxadiazol-3-yl]-1H-indazole trifluoroacetate and ethyl iodide were replaced with 3-methyl-1-[5-(piperidin-4-yl)-1,2,4-oxadiazol-3-yl]-1,4-indazole trifluoroacetate and methyl(3-chloropropyl)methylcarbamate, respectively, and the conversion step into hydrochloride was omitted.

LC-MS, m/z; 413 [M+H]+

### Preparations of Examples 270 to 271

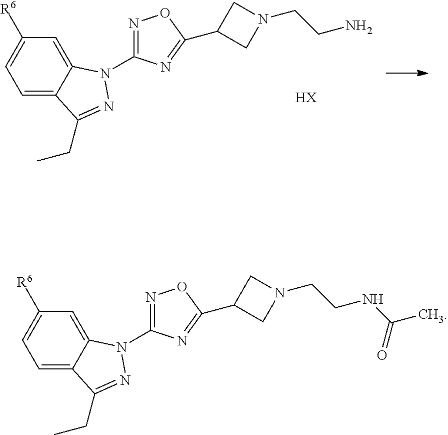

Wherein HX is hydrochloric acid or trifluoroacetic acid.

The compounds in the following table (i.e. Examples 270 to 271) were prepared in the same manner as in Example 265 except that the 2-{4-[3-(3-methyl-1H-indazol-1-yl)-1,2,4-oxadiazol-5-yl]piperidin-1-yl}ethanamine bis(trifluoroacetate) and benzoyl chloride were replaced with the corresponding starting compound and acetic anhydride, respectively.

TABLE 42

| | | | ¹H NMR/LC- |
|---|---|---|---|
| Ex. | R⁶ | Compound Name | MS, m/z |
| 270 | H | N-(2-{3-[3-(3-ethyl-1H-indazol-1- | LC-MS, m/z; |
| | | yl)-1,2,4-oxadiazol-5-yl]azetidin- | 355 [M + H]+ |
| | | 1-yl}ethyl)acetamide | |
| 271 | F | N-(2-{3-[3-(3-ethyl-6-fluoro-1H- | LC-MS, m/z; |
| | | indazol-1-yl)-1,2,4-oxadiazol-5- | 373 [M + H]+ |
| | | yl]azetidin-1-yl}ethyl)acetamide | |

### Examples 272 to 273

### Preparations of 3-ethyl-1-{5-[cis-4-(morpholin-4-yl)cyclohexyl]-1,2,4-oxadiazol-3-yl}-1H-indazole and 3-ethyl-1-{5-[trans-4-(morpholin-4-yl)cyclohexyl]-1,2,4-oxadiazol-3-yl}-1H-indazole

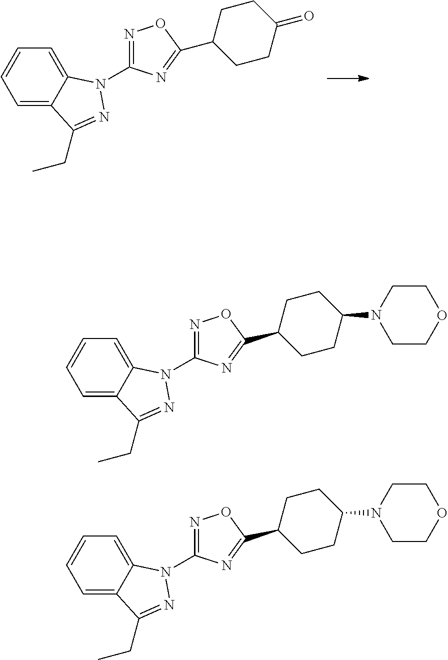 [SMILES: CCc1nn(-c2noc(C3CCC(=O)CC3)n2)c2ccccc12 ; CCc1nn(-c2noc([C@H]3CC[C@@H](N4CCOCC4)CC3)n2)c2ccccc12 ; CCc1nn(-c2noc([C@H]3CC[C@H](N4CCOCC4)CC3)n2)c2ccccc12]

4-[3-(3-Ethyl-1H-indazol-1-yl)-1,2,4-oxadiazol-5-yl]cyclohexanone (160 mg) was dissolved in dichloromethane (10 ml). To the solution was added morpholine (46 mg), and the mixture was stirred for 10 minutes. To the reaction mixture was further added acetic acid (40 mg), and the mixture was stirred for 30 minutes. To the resultant mixture was added triacetoxysodium borohydride (164 mg), and the mixture was stirred at room temperature overnight. After the completion of the reaction, 1N potassium hydroxide aqueous solution was added to the reaction mixture, and the mixture was extracted with ethyl acetate. The organic layer was washed with brine, dried over sodium sulfate and filtered, and the filtrate was concentrated under reduced pressure. The residue was purified by silica-gel chromatography (column; Hi-Flash™ Amino Column, developing solvent: hexane/ethyl acetate) to give the title compounds as a colorless oil of cis form: 75 mg and trans form: 30 mg, respectively.

cis form: ¹H-NMR (DMSO-d₆) δ: 1.36 (3H, t, J=7.6 Hz), 1.60-1.86 (6H, m), 2.10-2.30 (3H, m), 2.42 (2H, s), 3.03 (2H, q, J=7.6 Hz), 3.33-3.41 (3H, m), 3.55 (4H, m), 7.36-7.41 (1H, m), 7.62 (1H, m), 7.94 (1H, d, J=7.7 Hz), 8.20 (1H, d, J=8.3 Hz).

LC-MS, m/z; 382 [M+H]+

trans form: LC-MS, m/z; 382 [M+H]+

### Example 274

### Preparation of 3-ethyl-6-fluoro-1-{5-[cis-4-(pyrrolidin-1-yl)cyclohexyl]-1,2,4-oxadiazol-3-yl}-1H-indazole

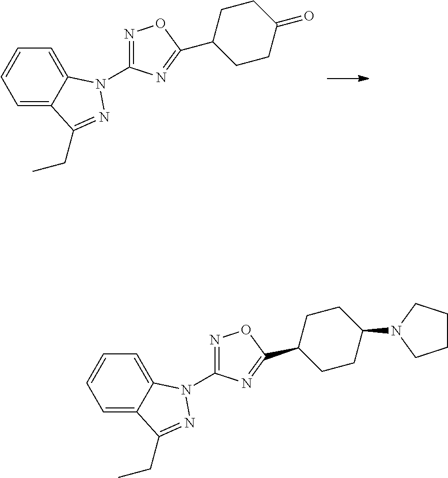 [SMILES: CCc1nn(-c2noc(C3CCC(=O)CC3)n2)c2ccccc12 ; CCc1nn(-c2noc([C@H]3CC[C@@H](N4CCCC4)CC3)n2)c2ccccc12]

The title compound was prepared in the same manner as in Example 272 except that the morpholine was replaced with pyrrolidine.

¹H-NMR (CDCl₃) δ: 1.44 (3H, t, J=7.7 Hz), 1.67-1.96 (10H, m), 2.13-2.23 (1H, m), 2.31-2.44 (2H, m), 2.48-2.61 (4H, m), 3.07 (2H, q, J=7.6 Hz), 3.16-3.26 (1H, m), 7.07 (1H, td, J=8.9, 2.2 Hz), 7.69 (1H, dd, J=8.7, 5.0 Hz), 7.98 (1H, dd, J=9.4, 2.3 Hz). LC-MS, m/z; 384 [M+H]+

### Preparations of Examples 275 to 278

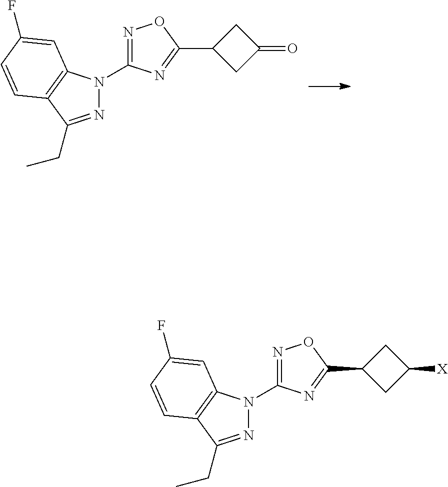

The compounds in the following table (i.e. Examples 275 to 278) were prepared in the same manner as in Example 272 except that the 4-[3-(3-ethyl-1H-indazol-1-yl)-1,2,4-oxadiazol-5-yl]cyclohexanone and morpholine were replaced with the corresponding starting compound “3-[3-(3-ethyl-6-fluoro-1H-indazol-1-yl)-1,2,4-oxadiazol-5-yl]cyclobutanone” and amine, respectively.

TABLE 43

| Ex. | X | Compound Name | ¹H-NMR/LC-MS, m/z |
|---|---|---|---|
| 275 | 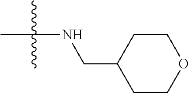 [SMILES: *NCC1CCOCC1] | cis-3-[3-(3-ethyl- 6-fluoro-1H- indazol-1-yl)- 1,2,4-oxadiazol-5- yl]-N-(tetrahydro- 2H-pyran-4-ylmethyl) cyclobutanamine | ¹H-NMR (CDCl₃) δ: 1.21- 1.58 (6H, m), 1.60-1.77 (3H, m), 2.28 (2H, m), 2.49 (2H, d, J = 6.2 Hz), 2.84 (2H, m), 3.07 (2H, q, J = 7.6 Hz), 3.32-3.55 (4H, m), 3.99 (2H, dd, J = 11.0, 3.9 Hz), 7.09 (1H, m), 7.71 (1H, m), 7.98 (1H, m). LC-MS, m/z; 400 [M + H]+ |
| 276 | 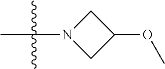 [SMILES: *N1CC(OC)C1] | 3-ethyl-6-fluoro- 1-{5-[3-(3- methoxyazetidin-1- yl)cyclobutyl]- 1,2,4-oxadiazol-3- yl}-1H-indazole | ¹H-NMR (CDCl₃) δ: 1.44 (3H, t, J = 7.6 Hz), 2.28- 2.67 (4H, m), 2.95-3.14 (4H, m), 3.25-3.67 (7H, m), 4.07 (1H, s), 7.08 (1H, m), 7.70 (1H, m), 7.98 (1H, m). LC-MS, m/z; 372 [M + H]+ |
| 277 | 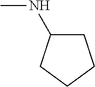 | N-{cis-3-[3-(3- ethyl-6-fluoro-1H- indazol-1-yl)- 1,2,4-oxadiazol-5- yl]cyclobutyl} cyclopentaneamine | ¹H-NMR (CDCl₃) δ: 1.38- 1.84 (9H, m), 2.13-2.40 (4H, m), 2.79 (2H, m), 3.07 (2H, q, J = 7.6 Hz), 3.23-3.54 (3H, m), 7.09 (1H, m), 7.70 (1H, m), 7.98 (1H, m). LC-MS, m/z; 370 [M + H]+ |
| 278 | 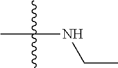 [SMILES: *NCC] | cis-N-ethyl-3-[3- (3-ethyl-6-fluoro- 1H-indazol-1-yl)- 1,2,4-oxadiazol-5- yl]cyclobutanamine | ¹H-NMR (CDCl₃) δ: 1.14 (3H, t, J = 7.2 Hz), 1.44 (3H, t, J = 7.6 Hz), 2.30 (2H, s), 2.67 (2H, q, J = 7.2 Hz), 2.85 (2H, s), 3.08 (2H, q, J = 7.6 Hz), 3.45 (2H, s), 7.09 (1H, m), 7.71 (1H, m), 7.98 (1H, m). LC-MS, m/z; 330 [M + H]+ |

### Preparations of Examples 279 to 281

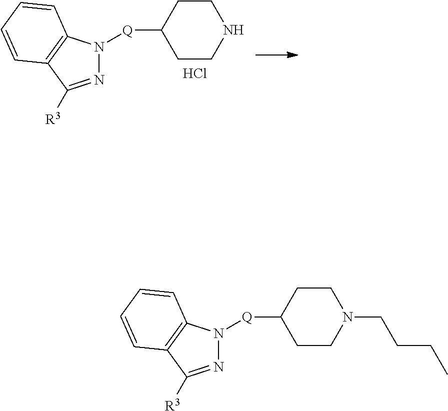

The compounds in the following table (i.e. Examples 279 to 281) were prepared in the same manner as in Example 097 except that the 3-ethyl-6-fluoro-1-[5-(piperidin-4-yl)-1,2,4-oxadiazol-3-yl]-1H-indazole trifluoroacetate and ethyl iodide were replaced with the corresponding starting compound and butyl bromide, respectively. Each free form of the compounds in the following table was obtained by omitting the conversion step into hydrochloride in Example 097.

TABLE 44

| | | | | ¹H-NMR/ |
|---|---|---|---|---|
| Ex. | R³ | Q | Compound Name | LC-MS, m/z |
| 279 | Cl | 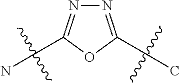 | 1-[5-(1-butylpiperidin-4- yl)-1,3,4-oxadiazol-2- yl]-3-chloro-1H-indazole | LC-MS, m/z; 360 [M + H]+ |
| 280 | Cl | 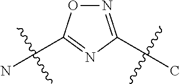 | 1-[3-(1-butylpiperidin-4- yl)-1,2,4-oxadiazol-5- yl]-3-chloro-1H-indazole hydrochloride | LC-MS, m/z; 360 [M + H]+ |
| 281 | ⁱPr | 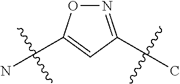 | 1-[3-(1-butylpiperidin-4- yl)isoxazol-5-yl]-3- (propan-2-yl)-1H-indazole | ¹H-NMR (DMSO-d₆) δ: 0.88 (3H, t, J = 7.3 Hz), 1.24-1.33 (2H, m), 1.37-1.45 (8H, m), 1.68-1.77 (2H, m), 1.89-2.01 (4H, m), 2.27 (2H, t, J = 7.3 Hz), 2.65-2.74 (1H, m), 2.91 (2H, d, J = 11.5 Hz), 3.43-3.50 (1H, m), 6.52 (1H, s), 7.37 (1H, t, J = 7.6 Hz), 7.60-7.64 (1H, m), 7.98-8.01 (2H, m). LC-MS, m/z; 367 [M + H]+ |

### Example 282

### Preparation of 1-{5-[1-(2-phenylethyl)piperidin-4-yl]-1,2,4-oxadiazol-3-yl}-1H-pyrrolo[2,3-b]pyridine

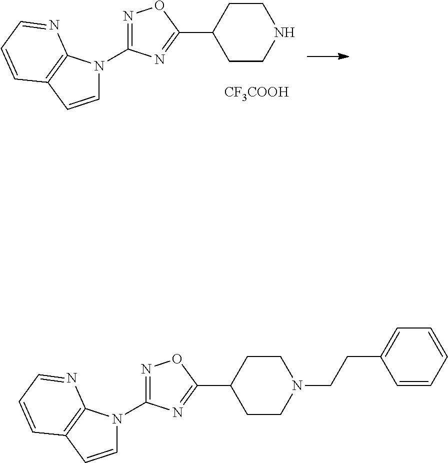 [SMILES: c1cnc2c(c1)ccn2-c1noc(C2CCNCC2)n1 ; "CF3COOH" ; c1ccc(CCN2CCC(c3nc(-n4ccc5cccnc54)no3)CC2)cc1]

The title compound was prepared in the same manner as in Example 097 except that the 3-ethyl-6-fluoro-1-[5-(piperidin-4-yl)-1,2,4-oxadiazol-3-yl]-1H-indazole trifluoroacetate and ethyl iodide were replaced with 1-[5-(piperidin-4-yl)-1,2,4-oxadiazol-3-yl]-1H-pyrrolo[2,3-b]pyridine trifluoroacetate and phenethyl bromide, respectively, and the conversion step into hydrochloride was omitted.

¹H-NMR (CDCl₃) δ: 1.99-2.33 (6H, m), 2.59-2.70 (2H, m), 2.79-2.89 (2H, m), 2.99-3.13 (3H, m), 6.69 (1H, d, J=3.9 Hz), 7.17-7.25 (4H, m), 7.26-7.33 (2H, m), 7.88 (1H, d, J=3.9 Hz), 7.96 (1H, m), 8.57 (1H, m).

LC-MS, m/z; 374 [M+H]+

### Preparations of Examples 283 to 284

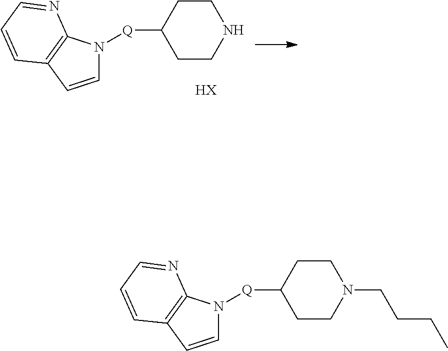

Wherein HX is hydrochloric acid or trifluoroacetic acid.

The compounds in the following table (i.e. Examples 283 to 284) were prepared in the same manner as in Example 097 except that the 3-ethyl-6-fluoro-1-[5-(piperidin-4-yl)-1,2,4-oxadiazol-3-yl]-1H-indazole trifluoroacetate and ethyl iodide were replaced with the corresponding starting compound and butyl bromide, respectively, and the conversion step into hydrochloride was omitted.

TABLE 45

| | | Compound | ¹H NMR/ |
|---|---|---|---|
| Ex. | Q | Name | LC-MS, m/z |
| 283 | 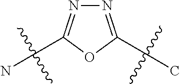 | 1-[3-(1- butylpiperidin- 4-yl)-1,2,4- oxadiazol-5-yl]- 1H-pyrrolo [2,3-b]pyridine | LC-MS, m/z; 326 [M + H]+ |
| 284 | 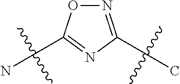 | 1-[5-(1- butylpiperidin- 4-yl)-1,3,4- oxadiazol-2-yl]- 1H-pyrrolo [2,3-b]pyridine | LC-MS, m/z; 326 [M + H]+ |

### Example 285

### Preparation of 1-{5-[1-(3-methoxypropyl)piperidin-4-yl]-1,2,4-oxadiazol-3-yl}-6-(propan-2-yl)-1H-pyrrolo[2,3-b]pyridine

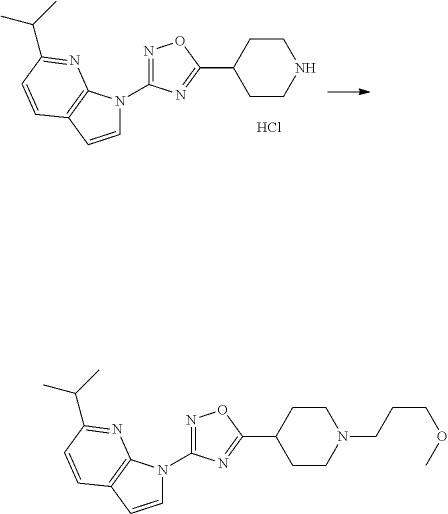 [SMILES: CC(C)c1ccc2ccn(-c3noc(C4CCNCC4)n3)c2n1 ; Cl ; COCCCN1CCC(c2nc(-n3ccc4ccc(C(C)C)nc43)no2)CC1]

The title compound was prepared in the same manner as in Example 085 except that the 1-[5-(piperidin-4-yl)-1,2,4-oxadiazol-3-yl]-3-(propan-2-yl)-1H-indazole hydrochloride was replaced with 1-[5-(piperidin-4-yl)-1,2,4-oxadiazol-3-yl]-6-(propan-2-yl)-1H-pyrrolo[2,3-b]pyridine hydrochloride.

¹H-NMR (CDCl₃) δ: 1.39 (6H, d, J=6.8 Hz), 1.79 (2H, m), 1.95-2.21 (6H, m), 2.45 (2H, dd, J=8.4, 6.8 Hz), 2.91-3.06 (3H, m), 3.27 (1H, m), 3.34 (3H, s), 3.43 (2H, t, J=6.4 Hz), 6.62 (1H, d, J=3.9 Hz), 7.14 (1H, d, J=8.1 Hz), 7.78 (1H, d, J=4.0 Hz), 7.86 (1H, d, J=8.1 Hz).

LC-MS, m/z; 384 [M+H]+

The compounds in the following table (i.e. Examples 286 to 297) were prepared in the same manner as in Example 001 or Example 012 except that the corresponding starting compound (which is described in Reference Examples 116 to 127) was used.

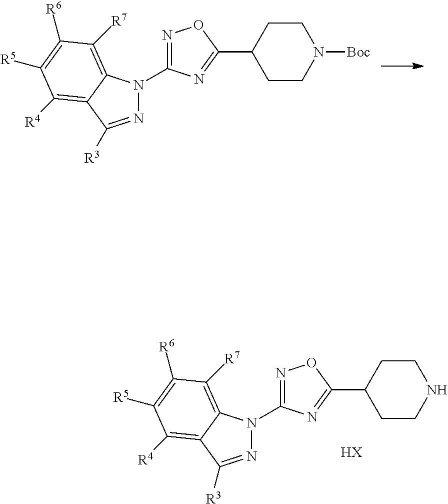

Wherein HX is hydrochloric acid or trifluoroacetic acid.

TABLE 46

| | | | | | | | ¹H-NMR/LC-MS, |
|---|---|---|---|---|---|---|---|
| Ex. | R³ | R⁴ | R⁵ | R⁶ | R⁷ | Compound Name | m/z |
| 286 | ⁱPr | Me | H | H | H | 4-methyl-1-[5- | LC-MS, m/z; |
| | | | | | | (piperidin-4- | 326 [M + H]+ |
| | | | | | | yl)-1,2,4- | |
| | | | | | | oxadiazol-3-yl]- | |
| | | | | | | 3-(propan-2-yl)- | |
| | | | | | | 1H-indazole | |
| | | | | | | hydrochloride | |
| 287 | ⁱPr | Cl | H | H | H | 4-chloro-1-[5- | LC-MS, m/z; |
| | | | | | | (piperidin-4- | 346 [M + H]+ |
| | | | | | | yl)-1,2,4- | |
| | | | | | | oxadiazol-3-yl]- | |
| | | | | | | 3-(propan-2-yl)- | |
| | | | | | | 1H-indazole | |
| | | | | | | hydrochloride | |
| 288 | ⁱPr | H | Me | H | H | 5-methyl-1-[5- | LC-MS, m/z; |
| | | | | | | (piperidin-4- | 326 [M + H]+ |
| | | | | | | yl)-1,2,4- | |
| | | | | | | oxadiazol-3-yl]- | |
| | | | | | | 3-(propan-2-yl)- | |
| | | | | | | 1H-indazole | |
| | | | | | | hydrochloride | |
| 289 | ⁱPr | H | Cl | H | H | 5-chloro-1-[5- | LC-MS, m/z; |
| | | | | | | (piperidin-4- | 346 [M + H]+ |
| | | | | | | yl)-1,2,4- | |
| | | | | | | oxadiazol-3-yl]- | |
| | | | | | | 3-(propan-2-yl)- | |
| | | | | | | 1H-indazole | |
| | | | | | | hydrochloride | |
| 290 | ⁱPr | H | MeO | H | H | 5-methoxy-1-[5- | LC-MS, m/z; |
| | | | | | | (piperidin-4- | 342 [M + H]+ |
| | | | | | | yl)-1,2,4- | |
| | | | | | | oxadiazol-3-yl]- | |
| | | | | | | 3-(propan-2-yl)- | |
| | | | | | | 1H-indazole | |
| | | | | | | hydrochloride | |
| 291 | Et | H | H | Me | H | 3-ethyl-6- | LC-MS, m/z; |
| | | | | | | methyl-1-[5- | 312 [M + H]+ |
| | | | | | | (piperidin-4- | |
| | | | | | | yl)-1,2,4- | |
| | | | | | | oxadiazol-3-yl]- | |
| | | | | | | 1H-indazole | |
| | | | | | | hydrochloride | |
| 292 | ⁱPr | H | H | Me | H | 6-methyl-1-[5- | LC-MS, m/z; |
| | | | | | | (piperidin-4- | 326 [M + H]+ |
| | | | | | | yl)-1,2,4- | |
| | | | | | | oxadiazol-3-yl]- | |
| | | | | | | 3-(propan-2-yl)- | |
| | | | | | | 1H-indazole | |
| | | | | | | hydrochloride | |
| 293 | ⁱPr | H | H | Cl | H | 6-chloro-1-[5- | LC-MS, m/z; |
| | | | | | | (piperidin-4- | 346 [M + H]+ |
| | | | | | | yl)-1,2,4- | |
| | | | | | | oxadiazol-3-yl]- | |
| | | | | | | 3-(propan-2-yl)- | |
| | | | | | | 1H-indazole | |
| | | | | | | hydrochloride | |
| 294 | ⁱPr | H | H | H | Me | 7-methyl-1-[5- | LC-MS, m/z; |
| | | | | | | (piperidin-4- | 326 [M + H]+ |
| | | | | | | yl)-1,2,4- | |
| | | | | | | oxadiazol-3-yl]- | |
| | | | | | | 3-(propan-2-yl)- | |
| | | | | | | 1H-indazole | |
| | | | | | | hydrochloride | |
| 295 | ⁱPr | H | H | H | MeO | 7-methoxy-1-[5- | LC-MS, m/z; |
| | | | | | | (piperidin-4- | 342 [M + H]+ |
| | | | | | | yl)-1,2,4- | |
| | | | | | | oxadiazol-3-yl]- | |
| | | | | | | 3-(propan-2-yl)- | |
| | | | | | | 1H-indazole | |
| | | | | | | hydrochloride | |
| 296 | Et | H | H | F | F | 3-ethyl-6,7- | LC-MS, m/z; |
| | | | | | | difluoro-1-[5- | 334 [M + H]+ |
| | | | | | | (piperidin-4- | |
| | | | | | | yl)-1,2,4- | |
| | | | | | | oxadiazol-3-yl]- | |
| | | | | | | 1H-indazole | |
| | | | | | | hydrochloride | |
| 297 | ⁱPr | H | H | F | F | 6,7-difluoro-1- | LC-MS, m/z; |
| | | | | | | [5-(piperidin-4- | 348 [M + H]+ |
| | | | | | | yl)-1,2,4- | |
| | | | | | | oxadiazol-3-yl]- | |
| | | | | | | 3-(propan-2-yl)- | |
| | | | | | | 1H-indazole | |
| | | | | | | hydrochloride | |

The compounds in the following table (i.e. Examples 298 to 307) were prepared in the same manner as in Example 001 or Example 012 except that the tert-butyl 4-[3-(3-ethyl-6-fluoro-1H-indazol-1-yl)-1,2,4-oxadiazol-5-yl]piperidine-1-carboxylate of Example 001 or the tert-butyl 4-{3-[3-(propan-2-yl)-1H-indazol-1-yl]-1,2,4-oxadiazol-5-yl}piperidine-1-carboxylate of Example 012 was replaced with the corresponding starting compound.

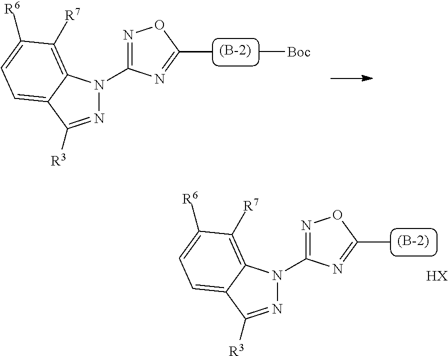

Wherein (B-2) means each cyclic amino structure shown in the following table, HX is hydrochloric acid or trifluoroacetic acid, and the Boc group is attached to the nitrogen atom in the cyclic amine of (B-2).

TABLE 47

| | | | | | | ¹H-NMR/LC-MS, |
|---|---|---|---|---|---|---|
| Ex. | R³ | R⁶ | R⁷ | (B-2) | Compound Name | m/z |
| 298 | ⁱPr | H | F | 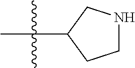 [SMILES: *C1CCNC1] | 7-fluoro-3-(propan- 2-yl)-1-[5- (pyrrolidin-3-yl)- 1,2,4-oxadiazol-3- yl]-1H-indazole hydrochloride | LC-MS, m/z; 316 [M + H]+ |
| 299 | ⁱPr | H | F | 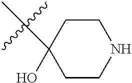 [SMILES: *C1(O)CCNCC1] | 4-{3-[7-fluoro-3- (propan-2-yl)-1H- indazol-1-yl]- 1,2,4-oxadiazol-5- yl}piperidin-4-ol hydrochloride | LC-MS, m/z; 346 [M + H]+ |
| 300 | ⁱPr | H | F | 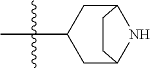 [SMILES: *C1CC2CCC(C1)N2] | 1-[5-(8- azabicyclo[3.2.1]oct- 3-yl)-1,2,4- oxadiazol-3-yl]-7- fluoro-3-(propan-2- yl)-1H-indazole trifluoroacetate | LC-MS, m/z; 356 [M + H]+ |
| 301 | ⁱPr | H | F | 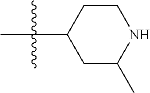 [SMILES: *C1CCNC(C)C1] | 7-fluoro-1-[5-(2- methylpiperidin-4- yl)-1,2,4- oxadiazol-3-yl]-3- (propan-2-yl)-1H- indazole trifluoroacetate | LC-MS, m/z; 344 [M + H]+ |
| 302 | ⁱPr | H | F | 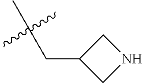 [SMILES: *CC1CNC1] | 1-[5-(azetidin-3- ylmethyl)-1,2,4- oxadiazol-3-yl]-7- fluoro-3-(propan-2- yl)-1H-indazole trifluoroacetate | LC-MS, m/z; 316 [M + H]+ |
| 303 | ⁱPr | H | F | 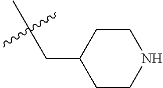 [SMILES: *CC1CCNCC1] | 7-fluoro-1-[5- (piperidin-4- ylmethyl)-1,2,4- oxadiazol-3-yl]-3- (propan-2-yl)-1H- indazole trifluoroacetate | LC-MS, m/z; 344 [M + H]+ |
| 304 | ⁱPr | H | F | 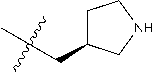 [SMILES: *C[C@H]1CCNC1] | 7-fluoro-3-(propan- 2-yl)-1-{5-[(3R)- pyrrolidin-3- ylmethyl]-1,2,4- oxadiazol-3-yl}-1H- indazole trifluoroacetate | LC-MS, m/z; 330 [M + H]+ |
| 305 | ⁱPr | H | F | 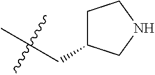 [SMILES: *C[C@@H]1CCNC1] | 7-fluoro-3-(propan- 2-yl)-1-{5-[(3S)- pyrrolidin-3- ylmethyl]-1,2,4- oxadiazol-3-yl}-1H- indazole trifluoroacetate | LC-MS, m/z; 330 [M + H]+ |
| 306 | Et | H | F | | 3-ethyl-7-fluoro-1- {5-[(3S)- pyrrolidin-3- ylmethyl]-1,2,4- oxadiazol-3-yl}-1H- indazole trifluoroacetate | No data |
| 307 | ⁱPr | F | H | | 6-fluoro-3-(propan- 2-yl)-1-{5-[(3S)- pyrrolidin-3- ylmethyl]-1,2,4- oxadiazol-3-yl}-1H- indazole trifluoroacetate | No data |

The compounds in the following table (i.e. Reference Examples 308 to 311) were prepared in the same manner as in Example 028 except that the 3-ethyl-1-[5-(piperidin-4-yl)-1,2,4-oxadiazol-3-yl]-1H-indazole trifluoroacetate and (S)-(−)-1-tert-butoxycarbonyl-2-pyrrolidinecarbaldehyde were replaced with the corresponding starting compound and tert-butyl 4-oxopiperidine-1-carboxylate, respectively.

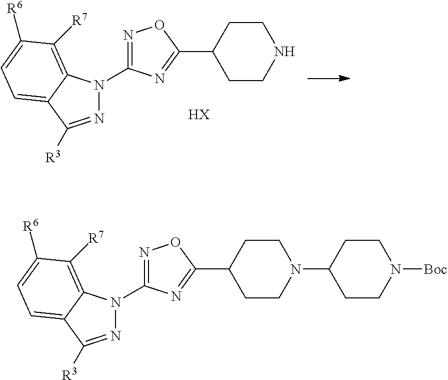

Wherein HX is hydrochloric acid or trifluoroacetic acid.

TABLE 48

| Ex. | R³ | R⁶ | R⁷ | Compound Name | LC-MS, m/z |
|---|---|---|---|---|---|
| 308 | ⁱPr | H | H | tert-butyl 4-{3-[3-(propan-2- | LC-MS, m/z; |
| | | | | yl)-1H-indazol-1-yl]-1,2,4- | 495 [M + H]+ |
| | | | | oxadiazol-5-yl}-1,4′- | |
| | | | | bipiperidine-1′-carboxylate | |
| 309 | ⁱPr | Me | H | tert-butyl 4-{3-[6-methyl-3- | LC-MS, m/z; |
| | | | | (propan-2-yl)-1H-indazol-1- | 509 [M + H]+ |
| | | | | yl]-1,2,4-oxadiazol-5-yl}- | |
| | | | | 1,4′-bipiperidine-1′- | |
| | | | | carboxylate | |
| 310¹⁾ | Et | F | F | tert-butyl 4-[3-(3-ethyl-6,7- | LC-MS, m/z; |
| | | | | difluoro-1H-indazol-1-yl)- | 517 [M + H]+ |
| | | | | 1,2,4-oxadiazol-5-yl]-1,4′- | |
| | | | | bipiperidine-1′-carboxylate | |
| 311¹⁾ | ⁱPr | F | F | tert-butyl 4-{3-[6,7-difluoro- | LC-MS, m/z; |
| | | | | 3-(propan-2-yl)-1H-indazol-1- | 531 [M + H]+ |
| | | | | yl]-1,2,4-oxadiazol-5-yl}- | |
| | | | | 1,4′-bipiperidine-1′- | |
| | | | | carboxylate | |

¹⁾Titanium tetraisopropoxide was added to the reaction system.

The compounds in the following table (i.e. Examples 312 to 315) were prepared in the same manner as in Example 028 except that the 3-ethyl-1-[5-(piperidin-4-yl)-1,2,4-oxadiazol-3-yl]-1H-indazole trifluoroacetate and (S)-(−)-1-tert-butoxycarbonyl-2-pyrrolidinecarbaldehyde were replaced with the corresponding starting compound and tert-butyl 3-formylazetidine-1-carboxylate, respectively.

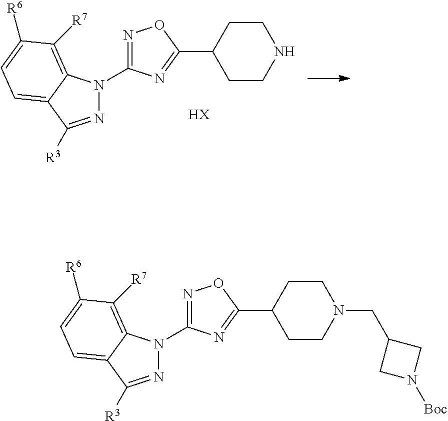

Wherein HX is hydrochloric acid or trifluoroacetic acid.

TABLE 49

| Ex. | R³ | R⁶ | R⁷ | Compound Name | LC-MS, m/z |
|---|---|---|---|---|---|
| 312 | ⁱPr | H | H | tert-butyl 3-[(4-{3-[3- | LC-MS, m/z; |
| | | | | (propan-2-yl)-1H-indazol-1- | 481 [M + H]+ |
| | | | | yl]-1,2,4-oxadiazol-5- | |
| | | | | yl}piperidin-1- | |
| | | | | yl)methyl]azetidine-1- | |
| | | | | carboxylate | |
| 313 | ⁱPr | Me | H | tert-butyl 3-[(4-{3-[6- | LC-MS, m/z; |
| | | | | methyl-3-(propan-2-yl)-1H- | 395 [M − tBu + H]+ |
| | | | | indazol-1-yl]-1,2,4- | |
| | | | | oxadiazol-5-yl}piperidin-1- | |
| | | | | yl)methyl]azetidine-1- | |
| | | | | carboxylate | |
| 314 | Et | F | F | tert-butyl 3-({4-[3-(3- | LC-MS, m/z; |
| | | | | ethyl-6,7-difluoro-1H- | 525 [M + Na]+ |
| | | | | indazol-1-yl)-1,2,4- | |
| | | | | oxadiazol-5-yl]piperidin-1- | |
| | | | | yl}methyl)azetidine-1- | |
| | | | | carboxylate | |
| 315 | ⁱPr | F | F | tert-butyl 3-[(4-{3-[6,7- | LC-MS, m/z; |
| | | | | difluoro-3-(propan-2-yl)- | 539 [M + Na]+ |
| | | | | 1H-indazol-1-yl]-1,2,4- | |
| | | | | oxadiazol-5-yl}piperidin-1- | |
| | | | | yl)methyl]azetidine-1- | |
| | | | | carboxylate | |

The compounds in the following table (i.e. Examples 316 to 319) were prepared in the same manner as in Example 028 except that the 3-ethyl-1-[5-(piperidin-4-yl)-1,2,4-oxadiazol-3-yl]-1H-indazole trifluoroacetate and (S)-(−)-1-tert-butoxycarbonyl-2-pyrrolidinecarbaldehyde were replaced with the corresponding starting compound and tert-butyl 4-formylpiperidine-1-carboxylate, respectively.

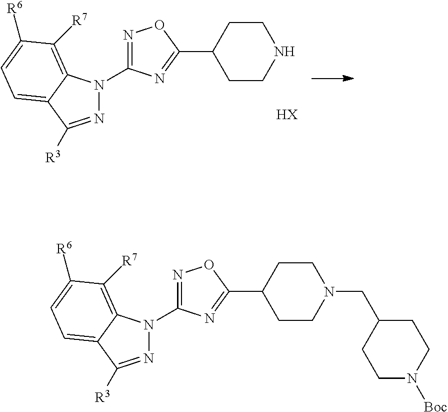

Wherein HX is hydrochloric acid or trifluoroacetic acid.

TABLE 50

| Ex. | R³ | R⁶ | R⁷ | Compound Name | LC-MS, m/z |
|---|---|---|---|---|---|
| 316 | ⁱPr | H | H | tert-butyl 4-[(4-{3-[3- | LC-MS, m/z; |
| | | | | (propan-2-yl)-1H-indazol-1- | 509 [M + H]+ |
| | | | | yl]-1,2,4-oxadiazol-5- | |
| | | | | yl}piperidin-1- | |
| | | | | yl)methyl]piperidine-1- | |
| | | | | carboxylate | |
| 317 | ⁱPr | Me | H | tert-butyl 4-[(4-{3-[6-methyl- | LC-MS, m/z; |
| | | | | 3-(propan-2-yl)-1H-indazol-1- | 523 [M + H]+ |
| | | | | yl]-1,2,4-oxadiazol-5- | |
| | | | | yl}piperidin-1- | |
| | | | | yl)methyl]piperidine-1- | |
| | | | | carboxylate | |
| 318 | Et | F | F | tert-butyl 4-({4-[3-(3-ethyl- | LC-MS, m/z; |
| | | | | 6,7-difluoro-1H-indazol-1-yl)- | 531 [M + H]+ |
| | | | | 1,2,4-oxadiazol-5- | |
| | | | | yl]piperidin-1- | |
| | | | | yl}methyl)piperidine-1- | |
| | | | | carboxylate | |
| 319 | ⁱPr | F | F | tert-butyl 4-[(4-{3-[6,7- | LC-MS, m/z; |
| | | | | difluoro-3-(propan-2-yl)-1H- | 545 [M + H]+ |
| | | | | indazol-1-yl]-1,2,4-oxadiazol- | |
| | | | | 5-yl}piperidin-1- | |
| | | | | yl)methyl]piperidine-1- | |
| | | | | carboxylate | |

### Example 320

### Preparation of tert-butyl 4-{3-[7-fluoro-3-(propan-2-yl)-1H-indazol-1-yl]-1,2,4-oxadiazol-5-yl}-4-hydroxy-1,4′-bipiperidine-1′-carboxylate

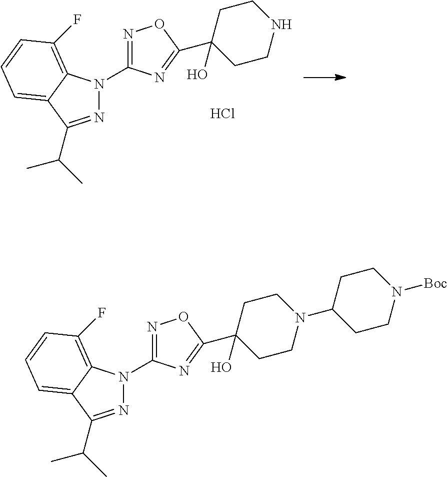 [SMILES: CC(C)c1nn(-c2noc(C3(O)CCNCC3)n2)c2c(F)cccc12 ; Cl ; CC(C)c1nn(-c2noc(C3(O)CCN(C4CCN(C(=O)OC(C)(C)C)CC4)CC3)n2)c2c(F)cccc12]

The title compound was prepared in the same manner as in Example 028 except that the 3-ethyl-1-[5-(piperidin-4-yl)-1,2,4-oxadiazol-3-yl]-1H-indazole trifluoroacetate and (S)-(−)-1-tert-butoxycarbonyl-2-pyrrolidinecarbaldehyde were replaced with 4-{3-[7-fluoro-3-(propan-2-yl)-1H-indazol-1-yl]-1,2,4-oxadiazol-5-yl}piperidin-4-ol hydrochloride (Example 299) and tert-butyl 4-oxopiperidine-1-carboxylate, respectively.

LC-MS, m/z; 529 [M+H]+

The compounds in the following table (i.e. Examples 321 to 325) were prepared in the same manner as in Example 028 except that the 3-ethyl-1-[5-(piperidin-4-yl)-1,2,4-oxadiazol-3-yl]-1H-indazole trifluoroacetate and (S)-(−)-1-tert-butoxycarbonyl-2-pyrrolidinecarbaldehyde were replaced with the corresponding starting compound and aldehyde or ketone, respectively.

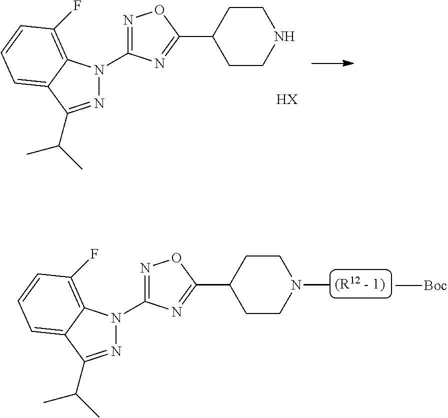

Wherein (R¹²-1) means each cyclic amino structure shown in the following table, HX is hydrochloric acid or trifluoroacetic acid, and the Boc group is attached to the nitrogen atom in the cyclic amine of (R¹²-1).

TABLE 51

| Ex. | (R¹²-1) | Compound Name | ¹H-NMR/LC-MS, m/z |
|---|---|---|---|
| 321 | 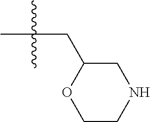 [SMILES: *CC1CNCCO1] | tert-butyl 2-[(4-{3-[7-fluoro-3- (propan-2-yl)-1H-indazol-1-yl]- 1,2,4-oxadiazol-5-yl}piperidin 1-yl)methyl]morphline-4- carboxylate | LC-MS, m/z; 529 [M + H]+ |
| 322¹⁾ | 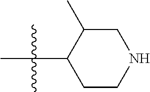 [SMILES: *C1CCNCC1C] | tert-butyl 4-{3-[7-fluoro-3- (propan-2-yl)-1H-indazol-1-yl]- 1,2,4-oxadiazol-5-yl}-3′-methyl- 1,4′-bipiperidine-1′-carboxylate | LC-MS, m/z; 527 [M + H]+ |
| 323¹⁾ | 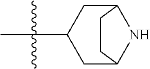 [SMILES: *C1CC2CCC(C1)N2] | tert-butyl 3-(4-{3-[7-fluoro-3- (propan-2-yl)-1H-indazol-1-yl]- 1,2,4-oxadiazol-5-yl}piperidin- 1-yl)-8-azabicyclo[3.2.1]octane- 8-carboxylate | LC-MS, m/z; 539 [M + H]+ |
| 324¹⁾ | 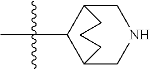 [SMILES: *C1C2CCCC1CNC2] | tert-butyl 9-(4-{3-[7-fluoro-3- (propan-2-yl)-1H-indazol-1-yl]- 1,2,4-oxadiazol-5-yl}piperidin- 1-yl)-3-azabicyclo[3.3.1]nonane- 3-carboxylate | LC-MS, m/z; 553 [M + H]+ |
| 325¹⁾ | 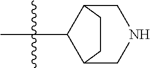 [SMILES: *C1C2CCC1CNC2] | tert-butyl 8-(4-{3-[7-fluoro-3- (propan-2-yl)-1H-indazol-1-yl]- 1,2,4-oxadiazol-5-yl}piperidin- 1-yl)-3-azabicyclo[3.2.1]octane- 3-carboxylate | LC-MS, m/z; 539 [M + H]+ |

¹⁾Titanium tetraisopropoxide was added to the reaction system.

The compounds in the following table (i.e. Examples 326 to 329) were prepared in the same manner as in Example 028 except that the 3-ethyl-1-[5-(piperidin-4-yl)-1,2,4-oxadiazol-3-yl]-1H-indazole trifluoroacetate and (S)-(−)-1-tert-butoxycarbonyl-2-pyrrolidinecarbaldehyde were replaced with 7-fluoro-3-(propan-2-yl)-1-{5-[(3R)-pyrrolidin-3-ylmethyl]-1,2,4-oxadiazol-3-yl}-1H-indazole trifluoroacetate (Example 304) and aldehyde or ketone, respectively.

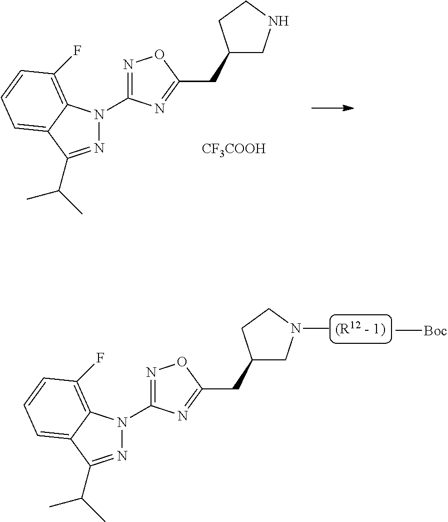

Wherein (R¹²-1) means each cyclic amino structure shown in the following table, and the Boc group is attached to the nitrogen atom in the cyclic amine of (R¹²-1).

TABLE 52

| Ex. | (R¹²-1) | Compound Name | ¹H-NMR/LC-MS, m/z |
|---|---|---|---|
| 326 | 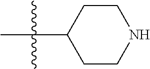 [SMILES: *C1CCNCC1] | tert-butyl 4-[(3R)-3-({3-[7- fluoro-3-(propan-2-yl)-1H- indazol-1-yl]-1,2,4-oxadiazol-5- yl}methyl)pyrrolidin-1- yl]piperidine-1-carboxylate | LC-MS, m/z; 513 [M + H]+ |
| 327 | 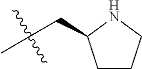 [SMILES: *C[C@@H]1CCCN1] | tert-butyl (2S)-2-{[(3R)-3-({3- [7-fluoro-3-(propan-2-yl)-1H- indazol-1-yl]-1,2,4-oxadiazol-5- yl}methyl)pyrrolidin-1- yl]methyl}pyrrolidine-1- carboxylate | LC-MS, m/z; 513 [M + H]+ |
| 328 | 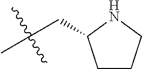 [SMILES: *C[C@H]1CCCN1] | tert-butyl (2R)-2-{[(3R)-3-({3- [7-fluoro-3-(propan-2-yl)-1H- indazol-1-yl]-1,2,4-oxadiazol-5- yl}methyl)pyrrolidin-1- yl]methyl}pyrrolidine-1- carboxylate | LC-MS, m/z; 513 [M + H]+ |
| 329 | 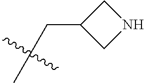 [SMILES: *CC1CNC1] | tert-butyl 3-{[(3R)-3-({3-[7- fluoro-3-(propan-2-yl)-1H- indazol-1-yl]-1,2,4-oxadiazol-5- yl}methyl)pyrrolidin-1- yl]methyl}azetidine-1-carboxylate | LC-MS, m/z; 499 [M + H]+ |

The compounds in the following table (i.e. Examples 330 to 333) were prepared in the same manner as in Example 028 except that the 3-ethyl-1-[5-(piperidin-4-yl)-1,2,4-oxadiazol-3-yl]-1H-indazole trifluoroacetate and (S)-(−)-1-tert-butoxycarbonyl-2-pyrrolidinecarbaldehyde were replaced with 7-fluoro-3-(propan-2-yl)-1-{5-[(3S)-pyrrolidin-3-ylmethyl]-1,2,4-oxadiazol-3-yl}-1H-indazole trifluoroacetate (Example 305) and aldehyde or ketone, respectively.

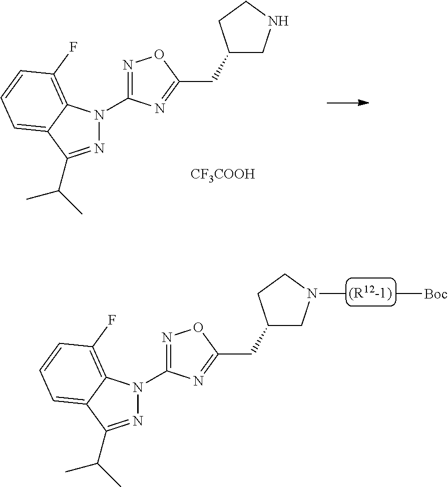

Wherein (R¹²-1) means each cyclic amino structure shown in the following table, and the Boc group is attached to the nitrogen atom in the cyclic amine of (R¹²-1).

TABLE 53

| | | | ¹H-NMR/ |
|---|---|---|---|
| | | | LC-MS, |
| Ex. | (R¹²-1) | Compound Name | m/z |
| 330 | 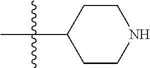 [SMILES: *C1CCNCC1] | tert-butyl 4- [(3S)-3-({3-[7- fluoro-3-(propan- 2-yl)-1H- indazol-1- yl]-1,2,4- oxadiazol- 5-yl}methyl) pyrrolidin- 1-yl]piperidine- 1-carboxylate | LC-MS, m/z; 513 [M + H]+ |
| 331 | 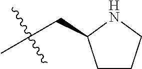 [SMILES: *C[C@@H]1CCCN1] | tert-butyl (2S)- 2-{[(3S)-3- ({3-[7-fluoro- 3-(propan- 2-yl)-1H- indazol-1- yl]-1,2,4- oxadiazol- 5-yl}methyl) pyrrolidin- 1-yl]methyl} pyrrolidine- 1-carboxylate | LC-MS, m/z; 513 [M + H]+ |
| 332 | 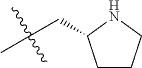 [SMILES: *C[C@H]1CCCN1] | tert-butyl (2R)- 2-{[(3S)-3- ({3-[7-fluoro- 3-(propan- 2-yl)-1H- indazol-1- yl]-1,2,4- oxadiazol- 5-yl}methyl) pyrrolidin- 1-yl]methyl} pyrrolidine- 1-carboxylate | LC-MS,m/z; 513 [M + H]+ |
| 333 | 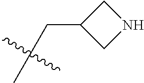 [SMILES: *CC1CNC1] | tert-butyl 3- {[(3S)-3-({3-[7- fluoro-3-(propan- 2-yl)-1H- indazol-1-yl]- 1,2,4-oxadiazol- 5-yl}methyl) pyrrolidin-1- yl]methyl} azetidine-1- carboxylate | LC-MS, m/z; 499 [M + H]+ |

### Example 334

### Preparation of tert-butyl 4-{3-[7-fluoro-3-(propan-2-yl)-1H-indazol-1-yl]-1,2,4-oxadiazol-5-yl}-1,4′-bipiperidine-1′-carboxylate

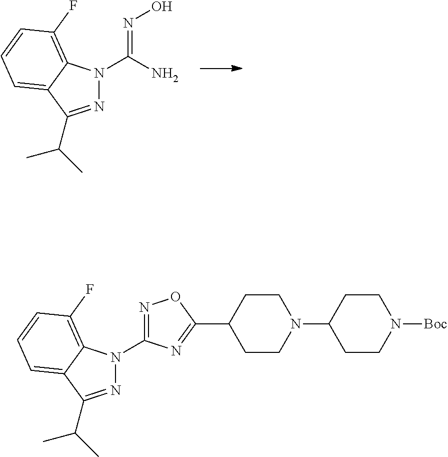 [SMILES: CC(C)c1nn(/C(N)=N/O)c2c(F)cccc12 ; CC(C)c1nn(-c2noc(C3CCN(C4CCN(C(=O)OC(C)(C)C)CC4)CC3)n2)c2c(F)cccc12]

1′-(Tert-butoxycarbonyl)-1,4′-bipiperidine-4-carboxylic acid (120 g) and triethylamine (124 ml) were suspended in THF (1000 ml). To the suspension was added dropwise isopropyl chlorocarbonate (47.2 g) at ice temperature, and the mixture was stirred at 40° C. for 1.5 hours. To the reaction mixture was added 7-fluoro-N′-hydroxy-3-(propan-2-yl)-1H-indazole-1-carboximidamide (70.0 g), and the mixture was stirred at 40° C. for 8 hours and further stirred at room temperature for 15 hours. To the reaction mixture was added saturated sodium bicarbonate (500 ml), and the mixture was stirred at room temperature for 30 minutes. To the mixture was further added saturated sodium bicarbonate (400 ml), and the resultant mixture was extracted with ethyl acetate (1500 ml). The organic layer was washed with saturated sodium bicarbonate (900 ml) and brine (900 ml), dried over anhydrous sodium sulfate, and concentrated under reduced pressure. The residue (149 g) was dissolved in toluene (1490 ml), 25% tetramethylammonium hydroxide aqueous solution (10.1 ml) was added thereto, and the mixture was stirred at 60° C. for 30 minutes. Then the reaction mixture was washed with water (1500 ml) and brine (1500 ml). The organic layer was dried over anhydrous sodium sulfate and concentrated under reduced pressure to give a quantitative amount of the title compound.

LC-MS, m/z; 513 [M+H]+

The compounds in the following table (i.e. Examples 335 to 341) were prepared in the same manner as in Example 334 except that the 7-fluoro-N′-hydroxy-3-(propan-2-yl)-1H-indazole-1-carboximidamide was replaced with the corresponding starting compound.

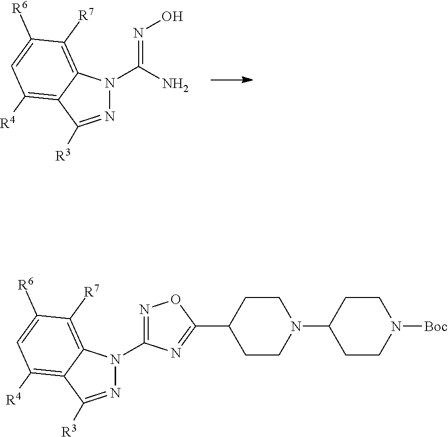

TABLE 54

| | | | | | | ¹H-NMR/ |
|---|---|---|---|---|---|---|
| Ex. | R³ | R⁴ | R⁶ | R⁷ | Compound Name | LC-MS, m/z |
| 335¹⁾ | 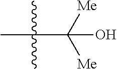 [SMILES: *C(C)(C)O] | H | H | F | tert-butyl 4-{3- [7-fluoro-3-(2- hydroxypropan-2- yl)-1H-indazol-1- yl]-1,2,4- oxadiazol-5-yl}- 1,4′-bipiperidine- 1′-carboxylate | LC-MS, m/z; 529 [M + H]+ |
| 336¹⁾ | ⁱPr | H | MeO | F | tert-butyl 4-{3- [7-fluoro-6- methoxy-3-(propan- 2-yl)-1H-indazol- 1-yl]-1,2,4- oxadiazol-5-yl}- 1,4′-bipiperidine- 1′-carboxylate | LC-MS, m/z; 543 [M + H]+ |
| 337¹⁾ | ⁱPr | MeO | H | F | tert-butyl 4-{3- [7-fluoro-4- methoxy-3-(propan- 2-yl)-1H-indazol- 1-yl]-1,2,4- oxadiazol-5-yl}- 1,4'-bipiperidine- 1′-carboxylate | LC-MS, m/z; 543 [M + H]+ |
| 338 | 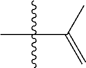 [SMILES: *C(=C)C] | H | H | F | tert-butyl 4-{3- [7-fluoro-3-(prop- 1-en-2-yl)-1H- indazol-1-yl]- 1,2,4-oxadiazol-5- yl}-1,4′- bipiperidine-1′- carboxylate | TLC Rf = 0.25 (hexane/ EtOAc = 3/1) |

¹⁾The cyclization reaction was carried out in the same manner as in Reference Example 33 except that the tetramethylammonium hydroxide aqueous solution was replaced with 1M tetra-butylammonium fluoride/THF.

TABLE 55

| | | | | | Compound | ¹H-NMR/ |
|---|---|---|---|---|---|---|
| Ex. | R³ | R⁴ | R⁶ | R⁷ | Name | LC-MS, m/z |
| 339 | 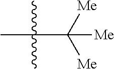 [SMILES: *C(C)(C)C] | H | H | H | tert-butyl 4- [3-(3-tert- butyl-1H- indazol-1-yl)- 1,2,4-oxadiazol- 5-yl]- 1,4′- bipiperidine-1′- carboxylate | LC-MS, m/z; 509 [M + H]+ |
| 340 | 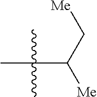 [SMILES: *C(C)CC] | H | H | F | tert-butyl 4- {3-[3- (butan-2-yl)- 7-fluoro- 1H-indazol- 1-yl]-1,2,4- oxadiazol- 5-yl}-1,4′- bipiperidine-1′- carboxylate | LC-MS, m/z; 527 [M + H]+ |
| 341 | I | H | H | F | tert-butyl 4- [3-(7- fluoro-3- iodo-1H- indazol-1- yl)-1,2,4- oxadiazol- 5-yl]-1,4′- bipiperidine-1′- carboxylate | LC-MS, m/z; 597 [M + H]+ |

The compounds in the following table (i.e. Examples 342 to 344) were prepared in the same manner as in Example 334 except that the 1′-(tert-butoxycarbonyl)-1,4′-bipiperidine-4-carboxylic acid was replaced with the corresponding carboxylic acid.

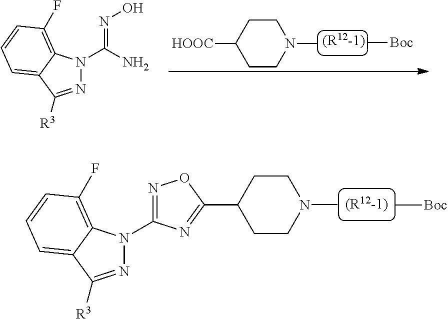

Wherein (R¹²-1) means each cyclic amino structure shown in the following table, and the Boc group is attached to the nitrogen atom in the cyclic amine of (R¹²-1).

TABLE 56

| | | | Compound | ¹H-NMR/ |
|---|---|---|---|---|
| Ex. | R³ | (R¹²-1) | Name | LC-MS, m/z |
| 342 | ⁱPr | 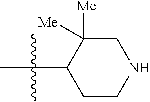 [SMILES: *C1CCNCC1(C)C] | tert-butyl 4- {3-[7-fluoro-3- (propan-2-yl)- 1H-indazol-1- yl]-1,2,4- oxadiazol-5-yl}- 3′,3′-dimethyl-1,4′- bipiperidine- 1′-carboxylate | No data |
| 343 | ⁱPr | 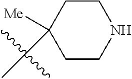 [SMILES: *C1(C)CCNCC1] | tert-butyl 4- {3-[7-fluoro-3- (propan-2-yl)- 1H-indazol-1- yl]-1,2,4- oxadiazol-5-yl}- 4′-methyl-1,4′- bipiperidine- 1′-carboxylate | LC-MS, m/z; 527 [M + H]+ |
| 344 | Et | | tert-butyl 4-[3- (3-ethyl-7- fluoro-1H- indazol-1-yl)- 1,2,4-oxadiazol- 5-yl]-4′- methyl-1,4′- bipiperidine-1′- carboxylate | LC-MS, m/z; 513 [M + H]+ |

### Example 345

### Preparation of tert-butyl 4-[3-(3-cyclopropyl-7-fluoro-1H-indazol-1-yl)-1,2,4-oxadiazol-5-yl]-1,4′-bipiperidine-1′-carboxylate

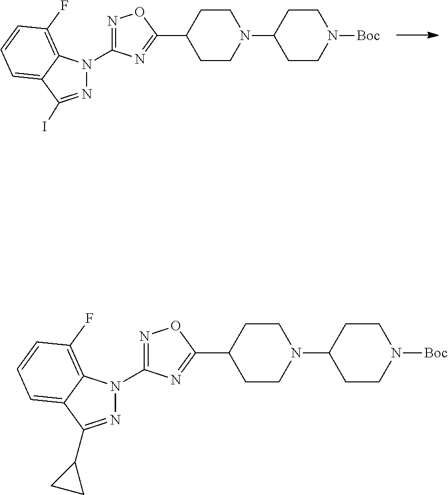 [SMILES: CC(C)(C)OC(=O)N1CCC(N2CCC(c3nc(-n4nc(I)c5cccc(F)c54)no3)CC2)CC1 ; CC(C)(C)OC(=O)N1CCC(N2CCC(c3nc(-n4nc(C5CC5)c5cccc(F)c54)no3)CC2)CC1]

Under nitrogen atmosphere, tert-butyl 4-[3-(7-fluoro-3-iodo-1H-indazol-1-yl)-1,2,4-oxadiazol-5-yl]-1,4′-bipiperidine-1′-carboxylate (100 mg), cyclopropylboronic acid (29 mg), potassium phosphate (107 mg), 1,1′-bis(diphenylphosphino)ferrocenepalladium dichloride (12 mg), water (0.3 ml) and toluene (2 ml) were mixed, and the mixture was stirred at 110° C. for 2.5 hours. The reaction solution was purified by amino column chromatography (eluate: hexane/ethyl acetate=100/0-0/100) to give the title compound (49 mg).

LC-MS, m/z; 511 [M+H]+

### Example 346

### Preparation of tert-butyl 4-[3-(7-fluoro-3-methyl-1H-indazol-1-yl)-1,2,4-oxadiazol-5-yl]-1,4′-bipiperidine-1′-carboxylate

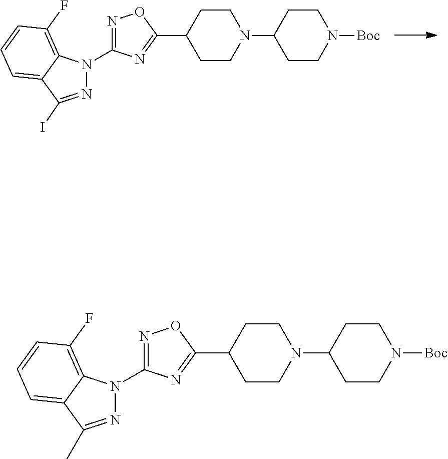 [SMILES: CC(C)(C)OC(=O)N1CCC(N2CCC(c3nc(-n4nc(I)c5cccc(F)c54)no3)CC2)CC1 ; Cc1nn(-c2noc(C3CCN(C4CCN(C(=O)OC(C)(C)C)CC4)CC3)n2)c2c(F)cccc12]

Under nitrogen atmosphere, tert-butyl 4-[3-(7-fluoro-3-iodo-1H-indazol-1-yl)-1,2,4-oxadiazol-5-yl]-1,4′-bipiperidine-1′-carboxylate (150 mg), 2 mol/L methyl zinc chloride/tetrahydrofuran (0.4 ml), bis(tri-tert-butylphosphine)palladium (26 mg) and tetrahydrofuran (1 ml) were mixed, and the mixture was stirred at room temperature for 3 hours. The reaction solution was purified by amino column chromatography (eluate: hexane/ethyl acetate=100/0-0/100) to give the title compound (64 mg).

LC-MS, m/z; 485 [M+H]+

The compounds in the following table (i.e. Examples 347 to 349) were prepared in the same manner as in Example 346 except that the methyl zinc chloride was replaced with the corresponding zinc reagent.

TABLE 57

| | | | ¹H-NMR/ |
|---|---|---|---|
| Ex. | R³ | Compound Name | LC-MS, m/z |
| 347 | ⁿPr | tert-butyl 4-[3-(7-fluoro-3- propyl-1H-indazol-1-yl)-1,2,4- oxadiazol-5-yl]-1,4′- bipiperidine-1′-carboxylate | LC-MS, m/z; 513 [M + H]+ |
| 348 | ⁱBu | tert-butyl 4-{3-[7-fluoro-3-(2- methylpropyl)-1H-indazol-1-yl]- 1,2,4-oxadiazol-5-yl}-1,4′- bipiperidine-1′-carboxylate | LC-MS, m/z; 527 [M + H]+ |
| 349 | 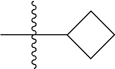 [SMILES: *C1CCC1] | tert-butyl 4-[3-(3-cyclobutyl-7- fluoro-1H-indazol-1-yl)-1,2,4- oxadiazol-5-yl]-1,4'- bipiperidine-1'-carboxylate | LC-MS, m/z; 525 [M + H]+ |

The compounds in the following table (i.e. Examples 350 to 351) were prepared in the same manner as in Example 035 except that the 3-ethyl-1-[5-(piperidin-4-yl)-1,2,4-oxadiazol-3-yl]-1H-indazole trifluoroacetate and tert-butyl (3R)-3-(iodomethyl)pyrrolidine-1-carboxylate were replaced with 7-fluoro-3-(propan-2-yl)-1-{5-[(3R)-pyrrolidin-3-ylmethyl]-1,2,4-oxadiazol-3-yl}-1H-indazole trifluoroacetate (Example 304) and tert-butyl (3R)-3-(iodomethyl)pyrrolidine-1-carboxylate or tert-butyl (3S)-3-(iodomethyl)pyrrolidine-1-carboxylate, respectively.

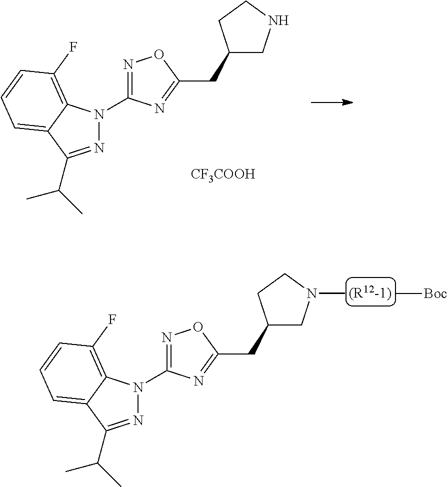

Wherein (R¹²-1) means each cyclic amino structure shown in the following table, and the Boc group is attached to the nitrogen atom in the cyclic amine of (R¹²-1).

TABLE 58

| | | Compound | ¹H-NMR/ |
|---|---|---|---|
| Ex. | (R¹²-1) | Name | LC-MS, m/z |
| 350 | 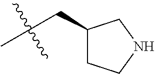 [SMILES: *C[C@@H]1CCNC1] | tert-butyl (3S)- 3-{[(3R)-3-({3- [7-fluoro-3- (propan-2-yl)-1H- indazol-1-yl]- 1,2,4-oxadiazol- 5-yl}methyl) pyrrolidin-1- yl]methyl} pyrrolidine-1- carboxylate | LC-MS, m/z; 513 [M + H]+ |
| 351 | 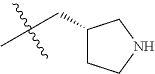 [SMILES: *C[C@H]1CCNC1] | tert-butyl (3R)- 3-{[(3R)-3-({3- [7-fluoro-3- (propan-2-yl)-1H- indazol-1-yl]- 1,2,4-oxadiazol- 5-yl}methyl) pyrrolidin-1- yl]methyl} pyrrolidine-1- carboxylate | LC-MS, m/z; 513 [M + H]+ |

The compounds in the following table (i.e. Examples 352 to 353) were prepared in the same manner as in Example 035 except that the 3-ethyl-1-[5-(piperidin-4-yl)-1,2,4-oxadiazol-3-yl]-1H-indazole trifluoroacetate and tert-butyl (3R)-3-(iodomethyl)pyrrolidine-1-carboxylate were replaced with 7-fluoro-3-(propan-2-yl)-1-{5-[(3S)-pyrrolidin-3-ylmethyl]-1,2,4-oxadiazol-3-yl}-1H-indazole trifluoroacetate and tert-butyl (3R)-3-(iodomethyl)pyrrolidine-1-carboxylate or tert-butyl (3S)-3-(iodomethyl)pyrrolidine-1-carboxylate, respectively.

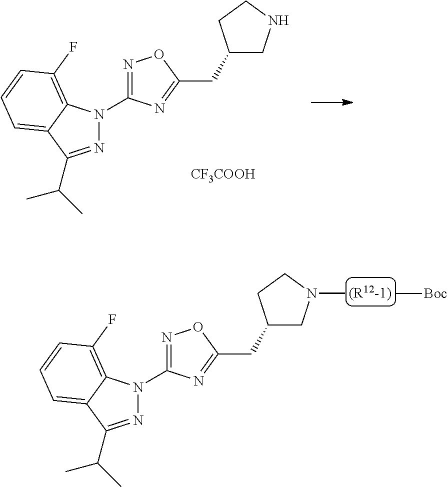

Wherein (R¹²-1) means each cyclic amino structure shown in the following table, and the Boc group is attached to the nitrogen atom in the cyclic amine of (R¹²-1).

TABLE 59

| | | | ¹H-NMR/ |
|---|---|---|---|
| Ex. | (R¹²-1) | Compound Name | LC-MS, m/z |
| 352 | 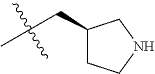 [SMILES: *C[C@@H]1CCNC1] | tert-butyl (3S)-3-{[(3S)- 3-({3-[7-fluoro-3-(propan- 2-yl)-1H-indazol-1-yl]- 1,2,4-oxadiazol-5- yl}methyl)pyrrolidin-1- yl]methyl}pyrrolidine-1- carboxylate | LC-MS, m/z; 513 [M + H]+ |
| 353 | 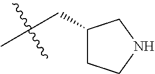 [SMILES: *C[C@H]1CCNC1] | tert-butyl (3R)-3-{[(3S)- 3-({3-[7-fluoro-3-(propan- 2-yl)-1H-indazol-1-yl]- 1,2,4-oxadiazol-5- yl}methyl)pyrrolidin-1- yl]methyl}pyrrolidine-1- carboxylate | LC-MS, m/z; 513 [M + H]+ |

The compounds in the following table (i.e. Examples 354 to 367) were prepared in the same manner as in Example 053 or Example 054 except that the tert-butyl 4-[(4-{3-[7-fluoro-3-(propan-2-yl)-1H-indazol-1-yl]-1,2,4-oxadiazol-5-yl}piperidin-1-yl)methyl]piperidine-1-carboxylate of Example 053 or the tert-butyl 3-[(4-{3-[7-fluoro-3-(propan-2-yl)-1H-indazol-1-yl]-1,2,4-oxadiazol-5-yl}piperidin-1-yl)methyl]azetidine-1-carboxylate of Example 054 was replaced with the corresponding starting compound.

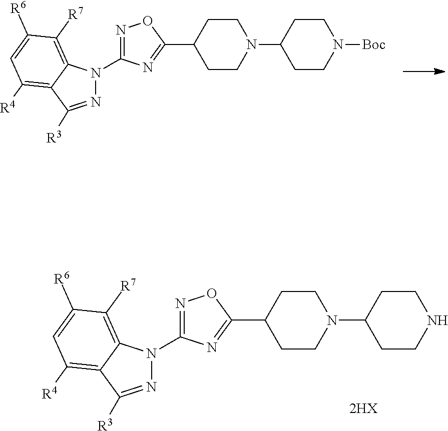

herein HX is hydrochloric acid or trifluoroacetic acid.

TABLE 60

| | | | | | | ¹H-NMR/ |
|---|---|---|---|---|---|---|
| Ex. | R³ | R⁴ | R⁶ | R⁷ | Compound Name | LC-MS, m/z |
| 354 | ⁱPr | H | H | H | 4-{3-[3-(propan-2- yl)-1H-indazol-1- yl]-1,2,4-oxadiazol- 5-yl}-1,4′- bipiperidine dihydrochloride | LC-MS, m/z; 395 [M + H]+ |
| 355 | 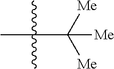 [SMILES: *C(C)(C)C] | H | H | H | 4-[3-(3-tert-butyl- 1H-indazol-1-yl)- 1,2,4-oxadiazol-5- yl]-1,4′- bipiperidine bis(trifluoroacetate) | LC-MS, m/z; 409 [M + H]+ |
| 356 | ⁱPr | H | Me | H | 4-{3-[6-methyl-3- (propan-2-yl)-1H- indazol-1-yl]-1,2,4- oxadiazol-5-yl}- 1,4′-bipiperidine dihydrochloride | LC-MS, m/z; 409 [M + H]+ |
| 357 | Et | H | F | F | 4-[3-(3-ethyl-6,7- difluoro-1H-indazol- 1-yl)-1,2,4- oxadiazol-5-yl]- 1,4′-bipiperidine dihydrochloride | LC-MS, m/z; 417 [M + H]+ |
| 358 | ⁱPr | H | F | F | 4-{3-[6,7-difluoro- 3-(propan-2-yl)-1H- indazol-1-yl]-1,2,4- oxadiazol-5-yl}- 1,4′-bipiperidine dihydrochloride | LC-MS,m/z; 431 [M + H]+ |
| 359 | ⁱPr | H | MeO | F | 4-{3-[7-fluoro-6- methoxy-3-(propan-2- yl)-1H-indazol-1- yl]-1,2,4-oxadiazol- 5-yl}-1,4′- bipiperidine bis(trifluoroacetate) | LC-MS, m/z; 443 [M + H]+ |
| 360 | ⁱPr | MeO | H | F | 4-{3-[7-fluoro-4- methoxy-3-(propan-2- yl)-1H-indazol-1- yl]-1,2,4-oxadiazol- 5-yl}-1,4′- bipiperidine bis(trifluoroacetate) | LC-MS, m/z; 443 [M + H]+ |
| 361 | 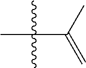 [SMILES: *C(=C)C] | H | H | F | 4-{3-[7-fluoro-3- (prop-1-en-2-yl)-1H- indazol-1-yl]-1,2,4- oxadiazol-5-yl}- 1,4′-bipiperidine bis(trifluoroacetate) | LC-MS, m/z; 411 [M + H]+ |
| 362 | 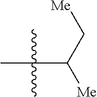 [SMILES: *C(C)CC] | H | H | F | 4-{3-[3-(butan-2- yl)-7-fluoro-1H- indazol-1-yl]-1,2,4- oxadiazol-5-yl}- 1,4′-bipiperidine dihydrochloride | LC-MS, m/z; 427 [M + H]+ |
| 363 | 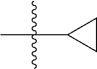 [SMILES: *C1CC1] | H | H | F | 4-[3-(3-cyclopropyl- 7-fluoro-1H-indazol- 1-yl)-1,2,4- oxadiazol-5-yl]- 1,4′-bipiperidine dihydrochloride | LC-MS, m/z; 411 [M + H]+ |
| 364 | ⁿPr | H | H | F | 4-[3-(7-fluoro-3- propyl-1H-indazol-1- yl)-1,2,4-oxadiazol- 5-yl]-1,4′- bipiperidine dihydrochloride | LC-MS, m/z; 413 [M + H]+ |
| 365 | Me | H | H | F | 4-[3-(7-fluoro-3- methyl-1H-indazol-1- yl)-1,2,4-oxadiazol- 5-yl]-1,4′- bipiperidine dihydrochloride | LC-MS, m/z; 385 [M + H]+ |
| 366 | ⁱBu | H | H | F | 4-{3-[7-fluoro-3-(2- methylpropyl)-1H- indazol-1-yl]-1,2,4- oxadiazol-5-yl}- 1,4′-bipiperidine dihydrochloride | LC-MS, m/z; 427 [M + H]+ |
| 367 | 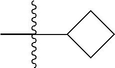 [SMILES: *C1CCC1] | H | H | F | 4-[3-(3-cyclobutyl- 7-fluoro-1H-indazol- 1-yl)-1,2,4- oxadiazol-5-yl]- 1,4′-bipiperidine dihydrochloride | LC-MS, m/z; 425 [M + H]+ |

The compounds in the following table (i.e. Examples 368 to 383) were prepared in the same manner as in Example 053 or Example 054 except that the tert-butyl 4-[(4-{3-[7-fluoro-3-(propan-2-yl)-1H-indazol-1-yl]-1,2,4-oxadiazol-5-yl}piperidin-1-yl)methyl]piperidine-1-carboxylate of Example 053 or the tert-butyl 3-[(4-{3-[7-fluoro-3-(propan-2-yl)-1H-indazol-1-yl]-1,2,4-oxadiazol-5-yl}piperidin-1-yl)methyl]azetidine-1-carboxylate of Example 054 was replaced with the corresponding starting compound.

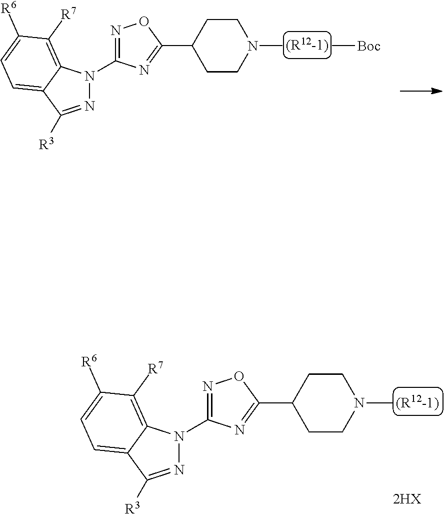

Wherein (R¹²-1) means each cyclic amino structure shown in the following table, the Hoc group is attached to the nitrogen atom in the cyclic amine of (R¹²-1), and HX is hydrochloric acid or trifluoroacetic acid.

TABLE 61

| Ex. | R³ | R⁶ | R⁷ | (R¹²-1) | Compound Name | LC-MS, m/z |
|---|---|---|---|---|---|---|
| 368 | ⁱPr | H | H | 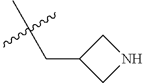 [SMILES: *CC1CNC1] | 1-{5-[1- (azetidin-3- ylmethyl)piperidin- 4-yl]-1,2,4- oxadiazol-3-yl}- 3-(propan-2-yl)- 1H-indazole bis(trifluoroacetate) | LC-MS, m/z; 381 [M + H]+ |
| 369 | ⁱPr | Me | H | | 1-{5-[1- (azetidin-3- ylmethyl)piperidin- 4-yl]-1,2,4- oxadiazol-3-yl}- 6-methyl-3- (propan-2-yl)- 1H-indazole bis(trifluoroacetate) | LC-MS, m/z; 395 [M + H]+ |
| 370 | Et | F | F | 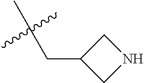 [SMILES: *CC1CNC1] | 1-{5-[1- (azetidin-3- ylmethyl)piperidin- 4-yl]-1,2,4- oxadiazol-3-yl}- 3-ethyl-6,7- difluoro-1H- indazole bis(trifluoroacetate) | No data |
| 371 | ⁱPr | F | F | | 1-{5-[1- (azetidin-3- ylmethyl)piperidin- 4-yl]-1,2,4- oxadiazol-3-yl}- 6,7-difluoro-3- (propan-2-yl)- 1H-indazole bis(trifluoroacetate) | No data |
| 372 | ⁱPr | H | H | 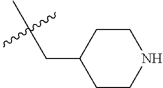 [SMILES: *CC1CCNCC1] | 1-{5-[1- (piperidin-4- ylmethyl)piperidin- 4-yl]-1,2,4- oxadiazol-3-yl}- 3-(propan-2-yl)- 1H-indazole bis(trifluoroacetate) | LC-MS, m/z; 409 [M + H]+ |
| 373 | ⁱPr | Me | H | | 6-methyl-1-{5- [1-(piperidin-4- ylmethyl)piperidin- 4-yl]-1,2,4- oxadiazol-3-yl}- 3-(propan-2-yl)- 1H-indazole bis(trifluoroacetate) | LC-MS, m/z; 423 [M + H]+ |
| 374 | Et | F | F | | 3-ethyl-6,7- difluoro-1-{5- [1-(piperidin-4- ylmethyl)piperidin- 4-yl]-1,2,4- oxadiazol-3-yl}- 1H-indazole dihydrochloride | LC-MS, m/z; 431 [M + H]+ |
| 375 | ⁱPr | F | F | | 6,7-difluoro-1- {5-[1- (piperidin-4- ylmethyl)piperidin- 4-yl]-1,2,4- oxadiazol-3-yl}- 3-(propan-2-yl)- 1H-indazole dihydrochloride | LC-MS, m/z; 445 [M + H]+ |
| 376 | ⁱPr | H | F | 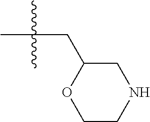 [SMILES: *CC1CNCCO1] | 7-fluoro-1-{5- [1-(morpholin-2- ylmethyl)piperidin- 4-yl]-1,2,4- oxadiazol-3-yl}- 3-(propan-2-yl)- 1H-indazole dihydrochloride | No data |
| 377 | ⁱPr | H | F | 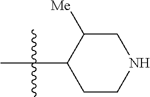 [SMILES: *C1CCNCC1C] | 4-{3-[7-fluoro- 3-(propan-2-yl)- 1H-indazol-1- yl]-1,2,4- oxadiazol-5-yl}- 3′-methyl-1,4′- bipiperidine bis(trifluoroacetate) | LC-MS, m/z; 427 [M + H]+ |
| 378 | ⁱPr | H | F | 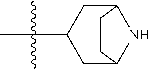 [SMILES: *C1CC2CCC(C1)N2] | 1-{5-[1-(8- azabicyclo[3.2.1] oct-3-yl)piperidin-4- yl]-1,2,4- oxadiazol-3-yl}- 7-fluoro-3- (propan-2-yl)- 1H-indazole bis(trifluoroacetate) | LC-MS, m/z; 439 [M + H]+ |
| 379 | ⁱPr | H | F | 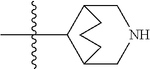 [SMILES: *C1C2CCCC1CNC2] | 1-{5-[1-(3- azabicyclo[3.3.1] non-9- yl)piperidin-4- yl]-1,2,4- oxadiazol-3-yl}- 7-fluoro-3- (propan-2-yl)- 1H-indazole bis(trifluoroacetate) | LC-MS, m/z; 453 [M + H]+ |
| 380 | ⁱPr | H | F | 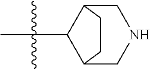 [SMILES: *C1C2CCC1CNC2] | 1-{5-[1-(3- azabicyclo[3.2.1] oct-8- yl)piperidin-4- yl]-1,2,4- oxadiazol-3-yl}- 7-fluoro-3- (propan-2-yl)- 1H-indazole bis(trifluoroacetate) | LC-MS, m/z; 439 [M + H]+ |
| 381 | ⁱPr | H | F | 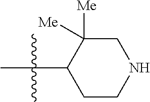 [SMILES: *C1CCNCC1(C)C] | 4-{3-[7-fluoro- 3-(propan-2-yl)- 1H-indazol-1- yl]-1,2,4- oxadiazol-5-yl}- 3′,3′-dimethyl- 1,4′- bipiperidine bis(trifluoroacetate) | No data |
| 382 | ⁱPr | H | F | 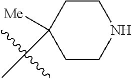 [SMILES: *C1(C)CCNCC1] | 4-{3-[7-fluoro- 3-(propan-2-yl)- 1H-indazol-1- yl]-1,2,4- oxadiazol-5-yl}- 4′-methyl-1,4′- bipiperidine bis(trifluoroacetate) | LC-MS, m/z; 427 [M + H]+ |
| 383 | Et | H | F | | 4-[3-(3-ethyl-7- fluoro-1H- indazol-1-yl)- 1,2,4-oxadiazol- 5-yl]-4′-methyl- 1,4′- bipiperidine bis(trifluoroacetate) | LC-MS, m/z; 413 [M + H]+ |

### Example 384

### Preparation of 4-{3-[7-fluoro-3-(propan-2-yl)-1H-indazol-1-yl]-1,2,4-oxadiazol-5-yl}-1,4′-bipiperidin-4-ol dihydrochloride

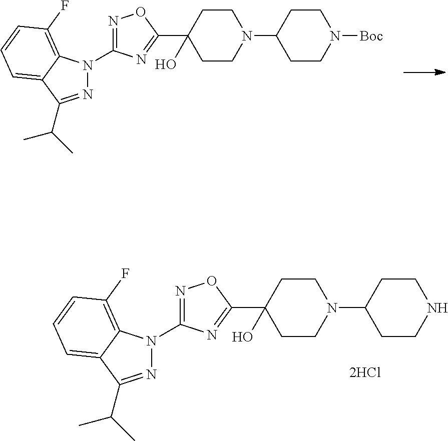 [SMILES: CC(C)c1nn(-c2noc(C3(O)CCN(C4CCN(C(=O)OC(C)(C)C)CC4)CC3)n2)c2c(F)cccc12 ; CC(C)c1nn(-c2noc(C3(O)CCN(C4CCNCC4)CC3)n2)c2c(F)cccc12]

The title compound was prepared in the same manner as in Example 053 except that the tert-butyl 4-[(4-{3-[7-fluoro-3-(propan-2-yl)-1H-indazol-1-yl]-1,2,4-oxadiazol-5-yl}piperidin-1-yl)methyl]piperidine-1-carboxylate was replaced with tert-butyl 4-{3-[7-fluoro-3-(propan-2-yl)-1H-indazol-1-yl]-1,2,4-oxadiazol-5-yl}-4-hydroxy-1,4′-bipiperidine-1′-carboxylate (Example 320).

LC-MS, m/z; 429 [M+H]+

The compounds in the following table (i.e. Examples 385 to 388) were prepared in the same manner as in Example 054 except that the tert-butyl 3-[(4-{3-[7-fluoro-3-(propan-2-yl)-1H-indazol-1-yl]-1,2,4-oxadiazol-5-yl}piperidin-1-yl)methyl]azetidine-1-carboxylate was replaced with the corresponding starting compound.

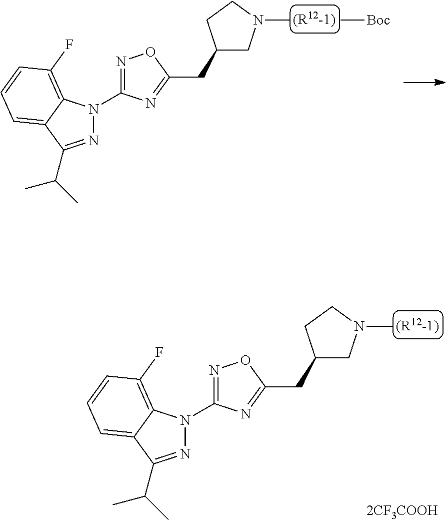

Wherein (R¹²-1) means each cyclic amino structure shown in the following table, and the Boc group is attached to the nitrogen atom in the cyclic amine of (R¹²-1).

TABLE 62

| Ex. | (R¹²-1) | Compound Name | LC-MS, m/z |
|---|---|---|---|
| 385 | 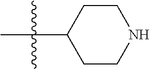 [SMILES: *C1CCNCC1] | 7-fluoro-1-(5-{[(3R)-1- (piperidin-4-yl)pyrrolidin- 3-yl]methyl}-1,2,4-oxa- diazol-3-yl)-3-(propan- 2-yl)-1H-indazole bis(trifluoroacetate) | LC-MS, m/z; 413 [M + H]+ |
| 386 | 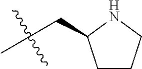 [SMILES: *C[C@@H]1CCCN1] | 7-fluoro-3-(propan-2-yl)- 1-[5-({(3R)-1-[(2S)- pyrrolidin-2-ylmethyl] pyrrolidin-3-yl}methyl)- 1,2,4-oxadiazol-3-yl]- 1H-indazole bis(trifluoro- acetate) | LC-MS, m/z; 413 [M + H]+ |
| 387 | 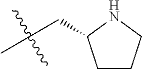 [SMILES: *C[C@H]1CCCN1] | 7-fluoro-3-(propan-2-yl)- 1-[5-({(3R)-1-[(2R)- pyrrolidin-2-ylmethyl] pyrrolidin-3-yl}methyl)- 1,2,4-oxadiazol-3-yl]- 1H-indazole bis(trifluoro- acetate) | LC-MS, m/z; 413 [M + H]+ |
| 388 | 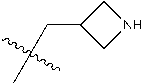 [SMILES: *CC1CNC1] | 1-(5-{[(3R)-1-(azetidin- 3-ylmethyl)pyrrolidin-3- yl]methyl}-1,2,4-oxa- diazol-3-yl)-7-fluoro-3- (propan-2-yl)-1H- indazole bis(trifluoro- acetate) | LC-MS, m/z; 399 [M + H]+ |

The compounds in the following table (i.e. Examples 389 to 392) were prepared in the same manner as in Example 054 except that the tert-butyl 3-[(4-{3-[7-fluoro-3-(propan-2-yl)-1H-indazol-1-yl]-1,2,4-oxadiazol-5-yl}piperidin-1-yl)methyl]azetidine-1-carboxylate was replaced with the corresponding starting compound.

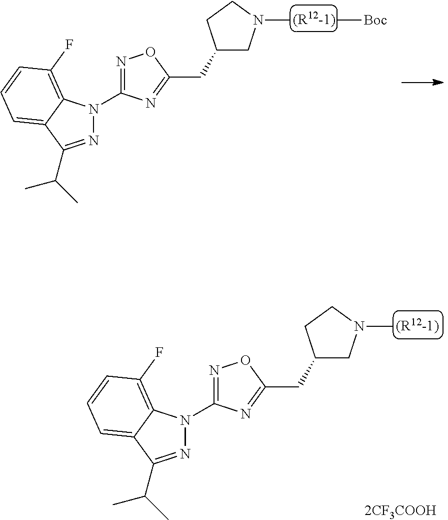

Wherein (R¹²-1) means each cyclic amino structure shown in the following table, and the Boc group is attached to the nitrogen atom in the cyclic amine of (R¹²-1).

TABLE 63

| Ex. | (R¹²-1) | Compound Name | LC-MS, m/z |
|---|---|---|---|
| 389 | 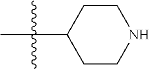 [SMILES: *C1CCNCC1] | 7-fluoro-1-(5-{[(3S)-1- (piperidin-4-yl)pyrrolidin- 3-yl]methyl}-1,2,4- oxadiazol-3-yl)-3-(propan- 2-yl)-1H-indazole bis(trifluoroacetate) | LC-MS, m/z; 413 [M + H]+ |
| 390 | 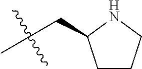 [SMILES: *C[C@@H]1CCCN1] | 7-fluoro-3-(propan-2-yl)-1- [5-({(3S)-1-[(2S)- pyrrolidin-2- ylmethyl]pyrrolidin-3- yl}methyl)-1,2,4-oxadiazol- 3-yl]-1H-indazole bis(trifluoroacetate) | LC-MS, m/z; 413 [M + H]+ |
| 391 | 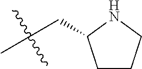 [SMILES: *C[C@H]1CCCN1] | 7-fluoro-3-(propan-2-yl)-1- [5-({(3S)-1-[(2R)- pyrrolidin-2- ylmethyl]pyrrolidin-3- yl}methyl)-1,2,4-oxadiazol- 3-yl]-1H-indazole bis(trifluoroacetate) | LC-MS, m/z; 413 [M + H]+ |
| 392 | 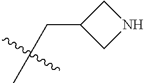 [SMILES: *CC1CNC1] | 1-(5-{[(3S)-1-(azetidin-3- ylmethyl)pyrrolidin-3- yl]methyl}-1,2,4-oxadiazol- 3-yl)-7-fluoro-3-(propan-2- yl)-1H-indazole bis(trifluoroacetate) | LC-MS, m/z; 399 [M + H]+ |

### Example 393

### Preparation of 1-(4-{3-[7-fluoro-3-(2-hydroxypropan-2-yl)-1H-indazol-1-yl]-1,2,4-oxadiazol-5-yl}-1,4′-bipiperidin-1′-yl)-2-hydroxyethanone

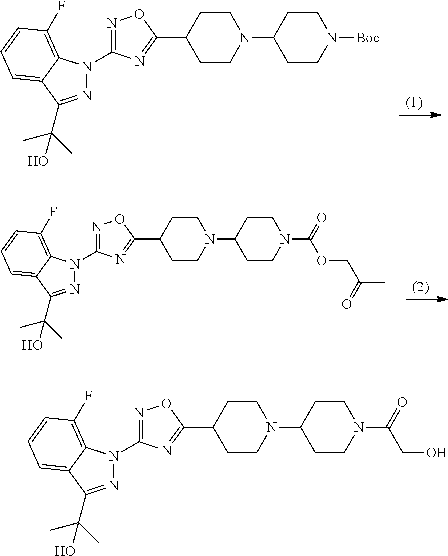 [SMILES: CC(C)(C)OC(=O)N1CCC(N2CCC(c3nc(-n4nc(C(C)(C)O)c5cccc(F)c54)no3)CC2)CC1 ; CC(=O)COC(=O)N1CCC(N2CCC(c3nc(-n4nc(C(C)(C)O)c5cccc(F)c54)no3)CC2)CC1 ; CC(C)(O)c1nn(-c2noc(C3CCN(C4CCN(C(=O)CO)CC4)CC3)n2)c2c(F)cccc12]

(1) Tert-butyl 4-{3-[7-fluoro-3-(2-hydroxypropan-2-yl)-1H-indazol-1-yl]-1,2,4-oxadiazol-5-yl}-1,4′-bipiperidine-1′-carboxylate (2.57 g) was dissolved in acetonitrile (125 ml). To the solution were added sodium iodide (2.33 g) and trimethylsilyl chloride (1.86 ml) under nitrogen atmosphere, and the mixture was stirred at room temperature for 2 hours. The reaction solution was cooled to −10° C. To the resultant were added sodium bicarbonate (4.09 g), water (75 ml), dichloromethane (115 ml) and acetoxyacetyl chloride (784 μl), and the mixture was stirred for 15 minutes. The organic layer was separated, washed with brine, dried, and the solvent was removed out. The residue was purified by silica-gel chromatography (column; Hi-Flash™, developing solvent: chloroform/methanol=10:1) to give 2-(4-{3-[7-fluoro-3-(2-hydroxypropan-2-yl)-1H-indazol-1-yl]-1,2,4-oxadiazol-5-yl}-1,4′-bipiperidin-1′-yl)-2-oxoethyl acetate (2.24 g).

(2) 2-(4-{3-[7-Fluoro-3-(2-hydroxypropan-2-yl)-1H-indazol-1-yl]-1,2,4-oxadiazol-5-yl}-1,4′-bipiperidin-1′-yl)-2-oxoethyl acetate (2.24 g) was dissolved in methanol (50 ml). To the solution was added methylamine (in 40% methanol, 1.72 ml), and the mixture was stirred at room temperature for 3 hours. The solvent was removed under reduced pressure, and the residue was recrystallized from 2-propanol (22 ml) to give the title compound (1.64 g) as a white crystal.

¹H-NMR (DMSO-d₆) δ: 1.20-1.48 (2H, m), 1.59-1.87 (11H, m), 2.10 (2H, d, J=10.5 Hz), 2.34 (2H, t, J=10.2 Hz), 2.49-2.67 (2H, m), 2.84-3.00 (3H, m), 3.09-3.22 (1H, m), 3.70 (1H, d, J=12.9 Hz), 4.07 (2H, t, J=6.1 Hz), 4.33-4.52 (2H, m), 7.32-7.51 (2H, m), 8.02 (1H, d, J=8.0 Hz).

LC-MS, m/z; 487 [M+H]+

### Example 394

### Preparation of 7-fluoro-1-{5-[3-(piperidin-1-yl)propyl]-1,2,4-oxadiazol-3-yl}-3-(propan-2-yl)-1H-indazole

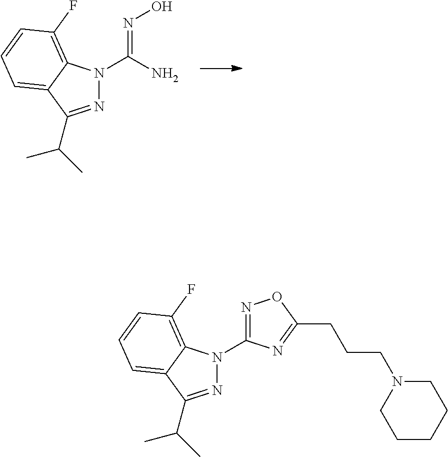 [SMILES: CC(C)c1nn(/C(N)=N/O)c2c(F)cccc12 ; CC(C)c1nn(-c2noc(CCCN3CCCCC3)n2)c2c(F)cccc12]

The title compound was prepared in the same manner as in Reference Example 044 except that the N′-hydroxy-3-(propan-2-yl)-1H-indazole-1-carboximidamide and 1-(tert-butoxycarbonyl)piperidine-4-carboxylic acid were replaced with 7-fluoro-N′-hydroxy-3-(propan-2-yl)-1H-indazole-1-carboximidamide and 4-(piperidin-1-yl)butanoic acid, respectively.

LC-MS, m/z; 372 [M+H]+

The compounds in the following table (i.e. Examples 395 to 400) were prepared in the same manner as in Example 097 except that the 3-ethyl-6-fluoro-1-[5-(piperidin-4-yl)-1,2,4-oxadiazol-3-yl]-1H-indazole trifluoroacetate and ethyl iodide were replaced with 7-fluoro-1-[5-(piperidin-4-yl)-1,2,4-oxadiazol-3-yl]-3-(propan-2-yl)-1H-indazole hydrochloride and R—X (which is an alkylating agent), respectively.

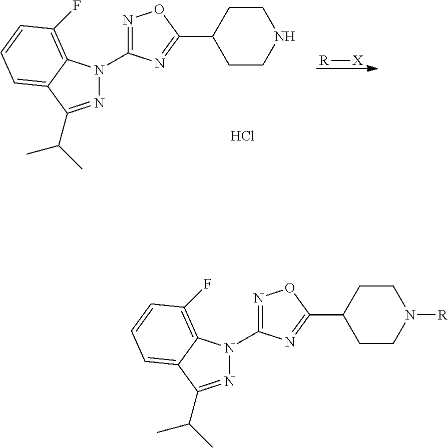

Each free form of the compounds in the following table was obtained by omitting the conversion step into hydrochloride in Example 097.

TABLE 64

| Ex. | R | Compound Name | LC-MS, m/z |
|---|---|---|---|
| 395 | 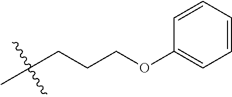 [SMILES: *CCCOc1ccccc1] | 7-fluoro-1-{5-[1-(3- phenoxypropyl)piperidin-4-yl]- 1,2,4-oxadiazol-3-yl}-3-(propan-2- yl)-1H-indazole | LC-MS, m/z; 464 [M + H]+ |
| 396 | 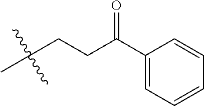 [SMILES: *CCC(=O)c1ccccc1] | 3-(4-{3-[7-fluoro-3-(propan-2-yl)- 1H-indazol-1-yl]-1,2,4-oxadiazol- 5-yl}piperidin-1-yl)-1- phenylpropan-1-one | LC-MS, m/z; 462 [M + H]+ |
| 397 | 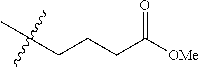 [SMILES: *CCCC(=O)OC] | methyl 4-(4-{3-[7-fluoro-3- (propan-2-yl)-1H-indazol-1-yl]- 1,2,4-oxadiazol-5-yl}piperidin-1- yl)butanoate | LC-MS, m/z; 430 [M + H]+ |
| 398 | 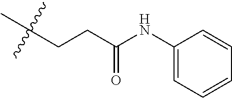 [SMILES: *CCC(=O)Nc1ccccc1] | 3-(4-{3-[7-fluoro-3-(propan-2-yl)- 1H-indazol-1-yl]-1,2,4-oxadiazol- 5-yl}piperidin-1-yl)-N- phenylpropanamide | LC-MS, m/z; 477 [M + H]+ |
| 399 | 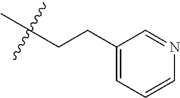 [SMILES: *CCc1cccnc1] | 7-fluoro-3-(propan-2-yl)-1-(5-{1- [2-(pyridin-3-yl)ethyl]piperidin- 4-yl}-1,2,4-oxadiazol-3-yl)-1H- indazole | LC-MS, m/z; 435 [M + H]+ |
| 400 | 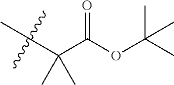 [SMILES: *C(C)(C)C(=O)OC(C)(C)C] | tert-butyl 2-(4-{3-[7-fluoro-3- (propan-2-yl)-1H-indazol-1-yl]- 1,2,4-oxadiazol-5-yl}piperidin-1- yl)-2-methylpropanoate | LC-MS, m/z; 472 [M + H]+ |

### Example 401

### Preparation of 2-(4-{3-[7-fluoro-3-(propan-2-yl)-1H-indazol-1-yl]-1,2,4-oxadiazol-5-yl}piperidin-1-yl)-2-methylpropanoic acid hydrochloride

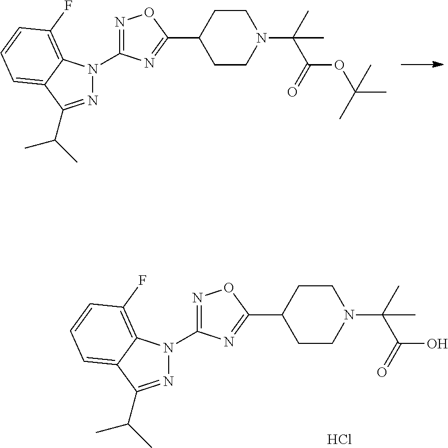 [SMILES: CC(C)c1nn(-c2noc(C3CCN(C(C)(C)C(=O)OC(C)(C)C)CC3)n2)c2c(F)cccc12 ; CC(C)c1nn(-c2noc(C3CCN(C(C)(C)C(=O)O)CC3)n2)c2c(F)cccc12 ; Cl]

A solution of tert-butyl 2-(4-{3-[7-fluoro-3-(propan-2-yl)-1H-indazol-1-yl]-1,2,4-oxadiazol-5-yl}piperidin-1-yl)-2-methylpropanoate (280 mg, 0.59 mmol) in 30 mL of 4 N HCl-dioxane was stirred at 60° C. for 2 hours. The solvent was removed in vacuo to give the crude, which was purified with preparative HPLC to give the pure product (220 mg, 89.1%) as a white solid as HCl salt.

LC-MS, m/z; 416 [M+H]+

### Example 402

### Preparation of 2-(4-{3-[7-fluoro-3-(propan-2-yl)-1-indazol-1-yl]-1,2,4-oxadiazol-5-yl}piperidin-1-yl)-2-methylpropan-1-ol

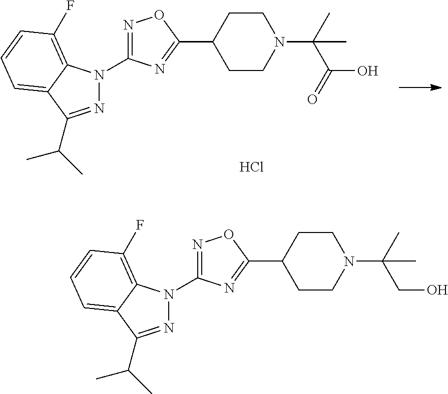 [SMILES: CC(C)c1nn(-c2noc(C3CCN(C(C)(C)C(=O)O)CC3)n2)c2c(F)cccc12 ; Cl ; CC(C)c1nn(-c2noc(C3CCN(C(C)(C)CO)CC3)n2)c2c(F)cccc12]

To a solution of 2-(4-{3-[7-fluoro-3-(propan-2-yl)-1H-indazol-1-yl]-1,2,4-oxadiazol-5-yl}piperidin-1-yl)-2-methylpropanoic acid hydrochloride (110 mg, 0.24 mmol) and triethylamine (0.07 mL, 0.48 mmol) in 5 mL of THF was added isobutyl chloroformate (0.03 mL, 0.26 mmol). The mixture was stirred at room temperature for 90 minutes. The white precipitation was filtered and to the filtrate was added dropwise a solution of sodium borohydride (46 mg, 1.2 mmol) in water (5 mL). The mixture was washed with aq. saturated NaHCO₃ (300 mL) and the organic layer was washed with brine, dried over Na₂SO₄ and evaporated in vacuo, which was purified with preparative HPLC to give the pure product (40.3 mg, 40.3%) as a light-yellow solid as free base.

LC-MS, m/z; 402 [M+H]+

The compounds in the following table (i.e. Examples 403 to 406) were prepared in the same manner as in Example 334 or the tetramethylammonium hydroxide aqueous solution was replaced with 1M tetrabutylammonium fluoride/THF solution, provided that that the 7-fluoro-N′-hydroxy-3-(propan-2-yl)-1H-indazole-1-carboximidamide and 1′-(tert-butoxycarbonyl)-1,4′-bipiperidine-4-carboxylic acid of Example 334 were replaced with the corresponding starting compound and 1′-acetyl-1,4′-bipiperidine-4-carboxylic acid, respectively.

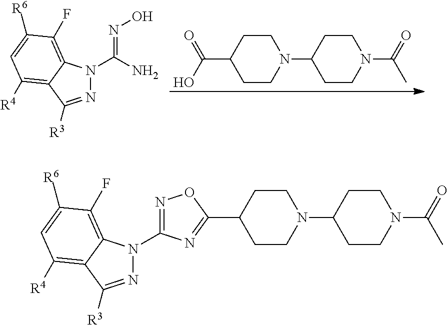

TABLE 65

| | | | | | ¹H-NMR/ |
|---|---|---|---|---|---|
| Ex. | R³ | R⁴ | R⁶ | Compound Name | LC-MS, m/z |
| 403 | 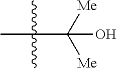 [SMILES: *C(C)(C)O] | H | H | 1-(4-{3-[7-fluoro- 3-(2-hydroxypro- pan-2-yl)-1H- indazol-1-yl]- 1,2,4-oxadiazol- 5-yl}-1,4′-bi- piperidin-1′- yl)ethanone | LC-MS, m/z; 471 [M + H]+ |
| 404 | ⁱPr | H | MeO | 1-(4-{3-[7-fluoro- 6-methoxy-3- (propan-2-yl)- 1H-indazol-1-yl]- 1,2,4-oxadiazol- 5-yl}-1,4′-bi- piperidin-1′-yl) ethanone | LC-MS, m/z; 485 [M + H]+ |
| 405 | ⁱPr | MeO | H | 1-(4-{3-[7- fluoro-4-methoxy- 3-(propan-2-yl)- 1H-indazol-1- yl]-1,2,4- oxadiazol-5-yl}- 1,4′-bipiperidin- 1′-yl)ethanone | LC-MS, m/z; 485 [M + H]+ |
| 406 | 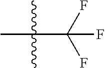 [SMILES: *C(F)(F)F] | H | H | 1-(4-{3-[7- fluoro-3-(trifluoro- methyl)-1H- indazol-1-yl]- 1,2,4-oxadiazol- 5-yl}-1,4′-bipiper- idin-1′-yl)ethanone | LC-MS, m/z; 481 [M + H]+ |

The compounds in the following table (i.e. Examples 407 to 414) were prepared in the same manner as in Example 028 except that the 3-ethyl-1-[5-(piperidin-4-yl)-1,2,4-oxadiazol-3-yl]-1H-indazole trifluoroacetate and (S)-(−)-1-tert-butoxycarbonyl-2-pyrrolidinecarbaldehyde were replaced with the corresponding starting compound and 1-acetylpiperidin-4-one, respectively.

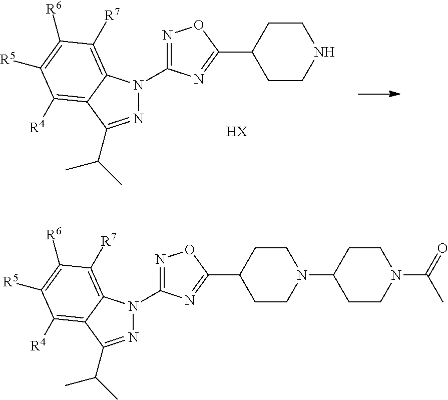

Wherein HX is hydrochloric acid or trifluoroacetic acid.

TABLE 66

| | | | | | | ¹H-NMR/ |
|---|---|---|---|---|---|---|
| Ex. | R⁴ | R⁵ | R⁶ | R⁷ | Compound Name | LC-MS, m/z |
| 407¹⁾ | Cl | H | H | H | 1-(4-{3-[4-chloro- | LC-MS, m/z; |
| | | | | | 3-(propan-2-yl)- | 493 [M + Na]+ |
| | | | | | 1H-indazol-1-yl]- | |
| | | | | | 1,2,4-oxadiazol-5- | |
| | | | | | yl}-1,4′- | |
| | | | | | bipiperidin-1′- | |
| | | | | | yl)ethanone | |
| 408¹⁾ | Me | H | H | H | 1-(4-{3-[4-methyl- | LC-MS, m/z; |
| | | | | | 3-(propan-2-yl)- | 473 [M + Na]+ |
| | | | | | 1H-indazol-1-yl]- | |
| | | | | | 1,2,4-oxadiazol-5- | |
| | | | | | yl}-1,4′- | |
| | | | | | bipiperidin-1′- | |
| | | | | | yl)ethanone | |
| 409¹⁾ | H | Cl | H | H | 1-(4-{3-[5-chloro- | LC-MS, m/z; |
| | | | | | 3-(propan-2-yl)- | 493 [M + Na]+ |
| | | | | | 1H-indazol-1-yl]- | |
| | | | | | 1,2,4-oxadiazol-5- | |
| | | | | | yl}-1,4′- | |
| | | | | | bipiperidin-1′- | |
| | | | | | yl)ethanone | |
| 410¹⁾ | H | Me | H | H | 1-(4-{3-[5-methyl- | LC-MS, m/z; |
| | | | | | 3-(propan-2-yl)- | 473 [M + Na]+ |
| | | | | | 1H-indazol-1-yl]- | |
| | | | | | 1,2,4-oxadiazol-5- | |
| | | | | | yl}-1,4′- | |
| | | | | | bipiperidin-1′- | |
| | | | | | yl)ethanone | |
| 411¹⁾ | H | MeO | H | H | 1-(4-{3-[5- | LC-MS, m/z; |
| | | | | | methoxy-3-(propan- | 489 [M + Na]+ |
| | | | | | 2-yl)-1H-indazol- | |
| | | | | | 1-yl]-1,2,4- | |
| | | | | | oxadiazol-5-yl}- | |
| | | | | | 1,4′-bipiperidin- | |
| | | | | | 1′-yl)ethanone | |
| 412¹⁾ | H | H | Cl | H | 1-(4-{3-[6-chloro- | LC-MS, m/z; |
| | | | | | 3-(propan-2-yl)- | 493 [M + Na]+ |
| | | | | | 1H-indazol-1-yl]- | |
| | | | | | 1,2,4-oxadiazol-5- | |
| | | | | | yl}-1,4′- | |
| | | | | | bipiperidin-1′- | |
| | | | | | yl)ethanone | |
| 413¹⁾ | H | H | H | Me | 1-(4-{3-[7-methyl- | LC-MS, m/z; |
| | | | | | 3-(propan-2-yl)- | 473 [M + Na]+ |
| | | | | | 1H-indazol-1-yl]- | |
| | | | | | 1,2,4-oxadiazol-5- | |
| | | | | | yl}-1,4′- | |
| | | | | | bipiperidin-1′- | |
| | | | | | yl)ethanone | |
| 414¹⁾ | H | H | H | MeO | 1-(4-{3-[7- | LC-MS, m/z; |
| | | | | | methoxy-3-(propan- | 467 [M + H]+ |
| | | | | | 2-yl)-1H-indazol- | |
| | | | | | 1-yl]-1,2,4- | |
| | | | | | oxadiazol-5-yl}- | |
| | | | | | 1,4′-bipiperidin- | |
| | | | | | 1′-yl)ethanone | |

¹⁾Titanium tetraisopropoxide was added to the reaction system.

The compounds in the following table (i.e. Examples 415 to 419) were prepared in the same manner as in Example 028 except that the 3-ethyl-1-[5-(piperidin-4-yl)-1,2,4-oxadiazol-3-yl]-1H-indazole trifluoroacetate and (S)-(−)-1-tert-butoxycarbonyl-2-pyrrolidinecarbaldehyde were replaced with the corresponding starting compound and 1-acetylpiperidin-4-one, respectively.

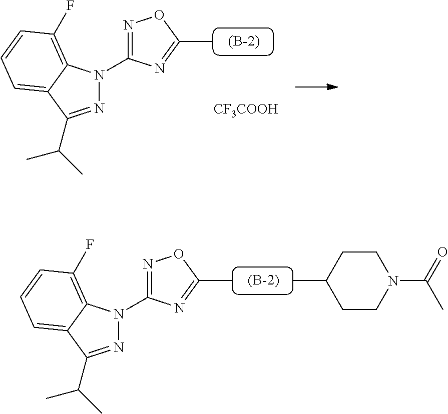

Wherein (B-2) means each cyclic amino structure shown in the following table, and the N-acetylpiperidine is attached to the nitrogen atom in the cyclic amine of (B-2).

TABLE 67

| Ex. | (B-2) | Compound Name | ¹H-NMR/LC-MS, m/z |
|---|---|---|---|
| 415¹⁾ | 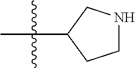 [SMILES: *C1CCNC1] | 1-[4-(3-{3-[7- fluoro-3-(propan- 2-yl)-1H-indazol- 1-yl]-1,2,4- oxadiazol-5- yl}pyrrolidin-1- yl)piperidin-1- yl]ethanone | LC-MS, m/z; 441 [M + H]+ |
| 416 | 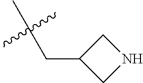 [SMILES: *CC1CNC1] | 1-{4-[3-([{3-[7- fluoro-3-(propan- 2-yl)-1H-indazol- 1-yl]-1,2,4- oxadiazol-5- yl}methyl)azetidin- 1-yl]piperidin-1- yl}ethanone | ¹H-NMR (CDCl₃) δ: 1.15-1.33 (2H, m), 1.50 (6H, d, J = 7.0 Hz), 1.57-1.73 (2H, m), 2.07 (3H, s), 2.20-2.31 (1H, m), 2.92-3.17 (5H, m), 3.27 (2H, d, J = 6.6 Hz), 3.42-3.53 (3H, m), 3.62- 3.74 (1H, m), 4.08-4.19 (1H, m), 7.18-7.29 (2H, m), 7.56-7.62 (1H, m). LC-MS, m/z; 441[M + H]+ |
| 417 | 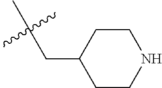 [SMILES: *CC1CCNCC1] | 1-[4-({3-[7- fluoro-3-(propan- 2-yl)-1H-indazol- 1-yl]-1,2,4- oxadiazol-5- yl}methyl)-1,4′- bipiperidin-1′- yl]ethanone | ¹H-NMR (CDCl₃) δ: 1.33-1.56 (10H, m), 1.77-2.04 (5H, m), 2.09 (3H, s), 2.15-2.30 (2H, m), 2.42-2.59 (2H, m), 2.85-3.10 (5H, m), 3.42- 3.54 (1H, m), 3.85 (1H, d, J = 12.1 Hz), 4.66 (1H, d, J = 12.1 Hz), 7.18-7.29 (2H, m), 7.56-7.62 (1H, m). LC-MS, m/z; 469 [M + H]+ |
| 418¹⁾ | 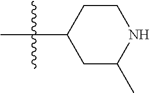 [SMILES: *C1CCNC(C)C1] | 1-(4-{3-[7-fluoro- 3-(propan-2-yl)- 1H-indazol-1-yl]- 1,2,4-oxadiazol-5- yl}-2-methyl-1,4′- bipiperidin-1′- yl}ethanone | ¹H-NMR (CDCl₃) δ: 1.09-1.15 (3H, m), 1.32-1.56 (7H, m), 1.74-2.00 (3H, m), 2.01- 2.29 (7H, m), 2.49-2.84 (4H, m), 2.96-3.28 (2H, m), 3.31-3.55 (2H, m), 3.76- 3.99 (1H, m), 4.48-4.65 (1H, m), 7.19-7.26 (2H, m), 7.56-7.62 (1H, m). LC-MS, m/z; 469 [M + H]+ |
| 419¹⁾ | 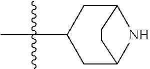 [SMILES: *C1CC2CCC(C1)N2] | 1-{4-[3-{3-[7- fluoro-3-(propan- 2-yl)-1H-indazol- 1-yl]-1,2,4- oxadiazol-5-yl}-8- azabicyclo[3.2.1] oct-8-yl]piperidin- 1-yl}ethanone | ¹H-NMR (CDCl₃) δ: 1.34-1.54 (8H, m), 1.56-1.97 (7H, m), 2.10 (3H, s), 2.27-2.45 (3H, m), 2.57-2.66 (1H, m), 2.77-2.87 (1H, m), 3.09- 3.18 (1H, m), 3.33-3.40 (1H, m), 3.44-3.56 (3H, m), 3.77-3.86 (1H, m), 4.39- 4.48 (1H, m), 7.18-7.26 (2H, m), 7.56-7.63 (1H, m). LC-MS, m/z; 481 [M + H]+ |

¹⁾Titanium tetraisopropoxide was added to the reaction system.

The compounds in the following table (i.e. Examples 420 to 454) were prepared in the same manner as in Example 134 except that the 3-ethyl-6-fluoro-1-[5-(piperidin-4-yl)-1,2,4-oxadiazol-3-yl]-1H-indazole trifluoroacetate and tetrahydropyrane-4-carboaldehyde were replaced with the corresponding starting compound and aldehyde or ketone, respectively.

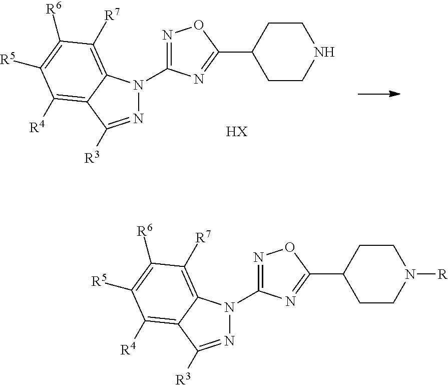

Wherein HX is hydrochloric acid or trifluoroacetic acid, and the structure of R is defined in the following table. In order to obtain each of the trifluoroacetates in the following table, the residue was isolated/purified by reverse phase HPLC.

TABLE 68

| | | | | | | | | ¹H-NMR/ |
|---|---|---|---|---|---|---|---|---|
| | | | | | | | | LC-MS, |
| Ex. | R³ | R⁴ | R⁵ | R⁶ | R⁷ | R | Compound Name | m/z |
| 420 | ⁱPr | H | H | H | H | 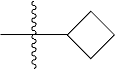 [SMILES: *C1CCC1] | 1-[5-(1- cyclobutyl- piperidin-4-yl)- 1,2,4- oxadiazol-3- yl]-3-(propan- 2-yl)-1H- indazole trifluoro- acetate | LC-MS, m/z; 366 [M + H]+ |
| 421 | ⁱPr | Cl | H | H | H | 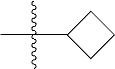 [SMILES: *C1CCC1] | 4-chloro-1-[5- (1- cyclobutyl- piperidin-4-yl)- 1,2,4- oxadiazol-3- yl]-3-(propan- 2-yl)-1H- indazole | LC-MS, m/z; 400 [M + H]+ |
| 422 | ⁱPr | Cl | H | H | H | 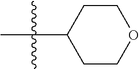 [SMILES: *C1CCOCC1] | 4-chloro-3- (propan-2-yl)- 1-{5-[1- (tetrahydro- 2H-pyran-4- yl)piperidin- 4-yl]-1,2,4- oxadiazol-3- yl}-1H- indazole | LC-MS, m/z; 430 [M + H]+ |
| 423 | ⁱPr | Me | H | H | H | 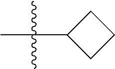 [SMILES: *C1CCC1] | 1-[5-(1- cyclobutyl- piperidin-4-yl)- 1,2,4- oxadiazol-3- yl]-4-methyl- 3-(propan-2- yl)-1H- indazole | LC-MS, m/z; 380 [M + H]+ |
| 424 | ⁱPr | Me | H | H | H | 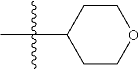 [SMILES: *C1CCOCC1] | 4-methyl-3- (propan-2-yl)- 1-{5-[1- (tetrahydro- 2H-pyran-4- yl)piperidin- 4-yl]-1,2,4- oxadiazol-3- yl}-1H- indazole | LC-MS, m/z; 410 [M + H]+ |
| 425 | ⁱPr | H | Cl | H | H | 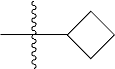 [SMILES: *C1CCC1] | 5-chloro-1-[5- (1- cyclobutyl- piperidin-4-yl)- 1,2,4- oxadiazol-3- yl]-3-(propan- 2-yl)-1H- indazole | LC-MS, m/z; 400 [M + H]+ |
| 426¹⁾ | ⁱPr | H | Cl | H | H | 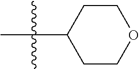 [SMILES: *C1CCOCC1] | 5-chloro-3- (propan-2-yl)- 1-{5-[1- (tetrahydro- 2H-pyran-4- yl)piperidin- 4-yl]-1,2,4- oxadiazol-3- yl}-1H- indazole | LC-MS, m/z; 430 [M + H]+ |
| 427 | ⁱPr | H | Me | H | H | 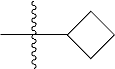 [SMILES: *C1CCC1] | 1-[5-(1- cyclobutyl- piperidin-4-yl)- 1,2,4- oxadiazol-3- yl]-5-methyl- 3-(propan-2- yl}-1H- indazole | LC-MS, m/z; 380 [M + H]+ |
| 428 | ⁱPr | H | Me | H | H | 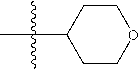 [SMILES: *C1CCOCC1] | 5-methyl-3- (propan-2-yl)- 1-{5-[1- (tetrahydro- 2H-pyran-4- yl)piperidin- 5-yl]-1,2,4- oxadiazol-3- yl}-1H- indazole | LC-MS, m/z; 410 [M + H]+ |
| 429 | ⁱPr | H | MeO | H | H | 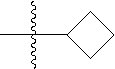 [SMILES: *C1CCC1] | 1-[5-(1- cyclobutyl- piperidin-4-yl)- 1,2,4- oxadiazol-3- yl]-5-methoxy- 3-(propan-2- yl)-1H- indazole | LC-MS, m/z; 396 [M + H]+ |
| 430 | ⁱPr | H | MeO | H | H | 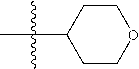 [SMILES: *C1CCOCC1] | 5-methoxy-3- (propan-2-yl)- 1-{5-[1- (tetrahydro- 2H-pyran-4- yl)piperidin- 4-yl]-1,2,4- oxadiazol-3- yl}-1H- indazole | LC-MS, m/z; 448 [M + Na]+ |
| 431 | ⁱPr | H | H | Cl | H | 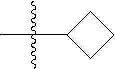 [SMILES: *C1CCC1] | 6-chloro-1-[5- (1- cyclobutyl- piperidin-4-yl)- 1,2,4- oxadiazol-3- yl]-3-(propan- 2-yl)-1H- indazole | LC-MS, m/z; 400 [M + H]+ |
| 432¹⁾ | ⁱPr | H | H | Cl | H | 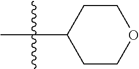 [SMILES: *C1CCOCC1] | 6-chloro-3- (propan-2-yl)- 1-{5-[1- (tetrahydro- 2H-pyran-4- yl)piperidin- 4-yl]-1,2,4- oxadiazol-3- yl}-1H- indazole | LC-MS, m/z; 430 [M + H]+ |
| 433 | ⁱPr | H | H | Me | H | 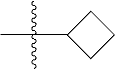 [SMILES: *C1CCC1] | 1-[5-(1- cyclobutyl- piperidin-4-yl)- 1,2,4- oxadiazol-3- yl]-6-methyl- 3-(propan-2- yl)-1H- indazole | LC-MS, m/z; 380 [M + H]+ |
| 434 | Et | H | H | Me | H | 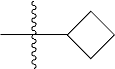 [SMILES: *C1CCC1] | 1-[5-(1- cyclobutyl- piperidin-4-yl)- 1,2,4- oxadiazol-3- yl]-3-ethyl-6- methyl-1H- indazole trifluoro- acetate | LC-MS, m/z; 366 [M + H]+ |
| 435 | Et | H | H | Me | H | 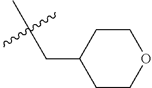 [SMILES: *CC1CCOCC1] | 3-ethyl-6- methyl-1-{5- [1- (tetrahydro- 2H-pyran-4- ylmethyl)piper- idin-4-yl]- 1,2,4- oxadiazol-3- yl}-1H- indazole trifluoro- acetate | LC-MS, m/z; 432 [M + Na]+ |
| 436 | ⁱPr | H | H | Me | H | 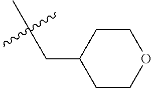 [SMILES: *CC1CCOCC1] | 6-methyl-3- (propan-2-yl)- 1-{5-[1- (tetrahydro- 2H-pyran-4- ylmethyl)piper- idin-4-yl]- 1,2,4- oxadiazol-3- yl}-1H- indazole trifluoro- acetate | LC-MS, m/z; 446 [M + Na]+ |
| 437 | Et | H | H | Me | H | 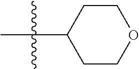 [SMILES: *C1CCOCC1] | 3-ethyl-6- methyl-1-{5- [1- (tetrahydro- 2H-pyran-4- yl)piperidin- 4-yl)-1,2,4- oxadiazol-3- yl}-1H- indazole trifluoro- acetate | LC-MS, m/z; 396 [M + H]+ |
| 438 | ⁱPr | H | H | H | Me | 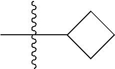 [SMILES: *C1CCC1] | 1-[5-(1- cyclobutyl- piperidin-4-yl)- 1,2,4- oxadiazol-3- yl]-7-methyl- 3-(propan-2- yl)-1H- indazole | LC-MS, m/z; 380 [M + H]+ |
| 439 | ⁱPr | H | H | H | Me | 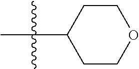 [SMILES: *C1CCOCC1] | 7-methyl-3- (propan-2-yl)- 1-{5-[1- (tetrahydro- 2H-pyran-4- yl)piperidin- 4-yl]-1,2,4- oxadiazol-3- yl}-1H- indazole | LC-MS, m/z; 432 [M + Na]+ |
| 440 | ⁱPr | H | H | H | MeO | 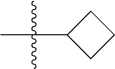 [SMILES: *C1CCC1] | 1-[5-(1- cyclobutyl- piperidin-4-yl)- 1,2,4- oxadiazol-3- yl]-7-methoxy- 3-(propan-2- yl)-1H- indazole | LC-MS, m/z; 396 [M + H]+ |
| 441¹⁾ | ⁱPr | H | H | H | MeO | 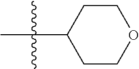 [SMILES: *C1CCOCC1] | 7-methoxy-3- (propan-2-yl)- 1-{5-[1- (tetrahydro- 2H-pyran-4- yl)piperidin- 4-yl]-1,2,4- oxadiazol-3- yl}-1H- indazole | LC-MS, m/z; 426 [M + H]+ |
| 442¹⁾ | ⁱPr | H | H | H | F | 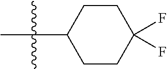 [SMILES: *C1CCC(F)(F)CC1] | 1-{5-[1-(4,4- difluorocyclo- hexyl)piperidin- 4-yl]-1,2,4- oxadiazol-3- yl}-7-fluoro- 3-(propan-2- yl)-1H- indazole | LC-MS, m/z; 448 [M + H]+ |
| 443¹⁾ | ⁱPr | H | H | H | F | 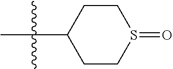 [SMILES: *C1CCS(=O)CC1] | 7-fluoro-1-{5- [1-(1- oxidotetra- hydro-2H- thiopyran-4- yl)piperidin- 4-yl]-1,2,4- oxadiazol-3- yl}-3-(propan- 2-yl)-1H- indazole | LC-MS, m/z; 446 [M + H]+ |
| 444¹⁾ | ⁱPr | H | H | H | F | 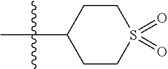 [SMILES: *C1CCS(=O)(=O)CC1] | 1-{5-[1-(1,1- dioxidotetra- hydro-2H- thiopyran-4- yl)piperidin- 4-yl]-1,2,4- oxadiazol-3- yl}-7-fluoro- 3-(propan-2- yl)-1H- indazole | LC-MS, m/z; 462 [M + H]+ |
| 445 | Et | H | H | F | F | ⁱPr | 3-ethyl-6,7- difluoro-1-{5- [1-(propan-2- yl)piperidin- 4-yl]-1,2,4- oxadiazol-3- yl}-1H- indazole trifluoro- acetate | ¹H-NMR (CDCl₃) δ: 1.30- 1.50 (9H, m), 2.34- 2.47 (1H, m), 2.48- 2.60 (1H, m), 2.61- 2.79 (1H, m), 2.95- 3.12 (2H, m), 3.12- 3.34 (2H, m), 3.42- 3.80 (5H, m), 7.12- 7.30 (1H, m), 7.40- 7.57 (1H, m). LC-MS, m/z; 376 [M + H]+ |
| 446 | Et | H | H | F | F | 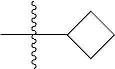 [SMILES: *C1CCC1] | 1-[5-(1- cyclobutyl- piperidin-4-yl)- 1,.2,4- oxadiazol-3- yl]-3-ethyl- 6,7-difluoro- 1H-indazole- trifluoro- acetate | ¹H-NMR (CDCl₃) δ: 1.43 (3H, t, J = 7.6 Hz), 1.65-1.93 (2H, m), 2.07-2.70 (10H, m), 2.92-3.12 (5H, m), 3.20-3.38 (1H, m), 7.12-7.24 (1H, m), 7.40-7.52 (1H, m). LC-MS, m/z; 388 [M + H]+ |
| 447 | Et | H | H | F | F | 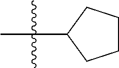 [SMILES: *C1CCCC1] | 1-[5-(1- cyclopentyl- piperidin-4-yl)- 1,2,4- oxadiazol-3- yl]-3-ethyl- 6,7-difluoro- 1H-indazole- trifluoro- acetate | ¹H-NMR (CDCl₃) δ: 1.15- 2.44 (17H, m), 2.50-2.70 (1H, m), 2.90-3.28 (5H, m), 7.07-7.23 (1H, m), 7.36-7.53 (1H, m). LC-MS, m/z; 402 [M + H]+ |
| 448 | Et | H | H | F | F | 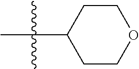 [SMILES: *C1CCOCC1] | 3-ethyl-6,7- difluoro-1-{5- [1- (tetrahydro- 2H-pyran-4- yl)piperidin- 4-yl]-1,2,4- oxadiazol-3- yl}-1H- indazole trifluoro- acetate | ¹H-NMR (CDCl₃) δ: 1.44 (3H, t, J = 7.5 Hz), 1.80-2.08 (4H, m), 2.31-2.98 (5H, m), 3.05 (2H, q, J = 7.5 Hz), 3.12-3.83 (7H, m), 4.04-4.20 (2H, m), 7.12-7.30 (1H, m), 7.40-7.55 (1H, m). LC-MS, m/z; 418 [M + H]+ |
| 449 | Et | H | H | F | F | 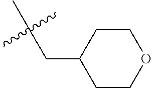 [SMILES: *CC1CCOCC1] | 3-ethyl-6,7- difluoro-1-{5- [1- (tetrahydro- 2H-pyran-4- ylmethyl)piper- idin-4-yl]- 1,2,4- oxadiazol-3- yl}-1H- indazole trifluoro- acetate | ¹H-NMR (CDCl₃) δ: 1.30 1.54 (5H, m), 1.69- 1.85 (2H, m), 2.07- 2.23 (1H, m), 2.30- 2.78 (4H, m), 2.78- 3.11 (5H, m), 3.11- 3.49 (4H, m), 3.59- 3.79 (2H, m), 3.88- 4.10 (2H, m), 7.12- 7.30 (1H, m), 7.40- 7.56 (1H, m). LC-MS, m/z; 432 [M + H]+ |
| 450 | ⁱPr | H | H | F | F | ⁱPr | 6,7-difluoro- 3-(propan-2- yl)-1-{5-[1- (propan-2- yl)piperidin- 4-yl]-1,2,4- oxadiazol-3- yl}-1H- indazole trifluoro- acetate | ¹H-NMR (CDCl₃) δ: 1.38- 1.51 (12H, m), 1.38-2.52 (2H, m), 2.52-2.70 (2H, m), 2.92-3.80 (7H, m), 7.12-7.25 (1H, m), 7.45-7.60 (1H, m). LC-MS, m/z; 390 [M + H]+ |
| 451 | ⁱPr | H | H | F | F | 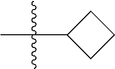 [SMILES: *C1CCC1] | 1-[5-(1- cyclobutyl- piperidin-4-yl)- 1,2,4- oxadiazol-3- yl]-6,7- difluoro-3- (propan-2-yl)- 1H-indazole- trifluoro- acetate | ¹H-NMR (CDCl₃) δ: 1.35- 1.52 (6H, m), 1.72- 2.05 (2H, m), 2.24- 2.63 (8H, m), 2.76- 3.15 (2H, m), 3.30- 3.75 (5H, m), 7.09- 7.25 (1H, m), 7.43- 7.59 (1H, m). LC-MS, m/z; 402 [M + H]+ |
| 452 | ⁱPr | H | H | F | F | 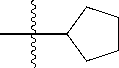 [SMILES: *C1CCCC1] | 1-[5-(1- cyclopentyl- piperidin-4-yl)- 1,2,4- oxadiazol-3- yl]-6,7- difluoro-3- (propan-2-yl)- 1H-indazole trifluoro- acetate | ¹H-NMR (CDCl₃) δ: 1.45 (6H, d, J = 6.8 Hz), 1.49-1.65 (2H, m), 1.65-1.85 (4H, m), 1.85-2.02 (2H, m), 2.18-2.30 (2H, m), 2.40-2.95 (5H, m), 3.10-3.32 (3H, m), 3.42 (1H, m, J = 6.8 Hz), 7.10-7.20 (1H, m), 7.45-7.55 (1H, m). LC-MS, m/z; 416 [M + H]+ |
| 453 | ⁱPr | H | H | F | F | 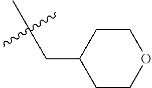 [SMILES: *CC1CCOCC1] | 6,7-difluoro- 3-(propan-2- yl)-1-{5-[1- (tetrahydro- 2H-pyran-4- ylmethyl)piper- idin-4-yl]- 1,2,4- oxadiazol-3- yl}-1H- indazole trifluoro- acetate | LC-MS, m/z; 446 [M + H]+ |
| 454 | ⁱPr | H | H | F | F | 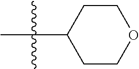 [SMILES: *C1CCOCC1] | 6,7-difluoro- 3-(propan-2- yl)-1-{5-[1- (tetrahydro- 2H-pyran-4- yl)piperidin- 4-yl]-1,2,4- oxadiazol-3- yl}-1H- indazole trifluoro- acetate | ¹H-NMR (CDCl₃) δ: 1.47 (6H, d, J = 6.8 Hz), 1.65-1.80 (2H, m), 1.86-1.98 (2H, m), 2.12-2.28 (2H, m), 2.37-2.53 (2H, m), 2.63-2.91 (3H, m), 3.09-3.30 (3H, m), 3.34-3.50 (3H, m), 4.00-4.10 (2H, m), 7.09-7.20 (1H, m), 7.45-7.55 (1H, m). LC-MS, m/z; 432 [M + H]+ |

¹⁾Titanium tetraisopropoxide was added to the reaction system.

The compounds in the following table (i.e. Examples 455 to 456) were prepared in the same manner as in Example 134 except that the 3-ethyl-6-fluoro-1-[5-(piperidin-4-yl)-1,2,4-oxadiazol-3-yl]-1H-indazole trifluoroacetate and tetrahydropyrane-4-carboaldehyde were replaced with 7-fluoro-3-(propan-2-yl)-1-[5-(pyrrolidin-3-yl)-1,2,4-oxadiazol-3-yl]-1H-indazole hydrochloride and aldehyde or ketone, respectively.

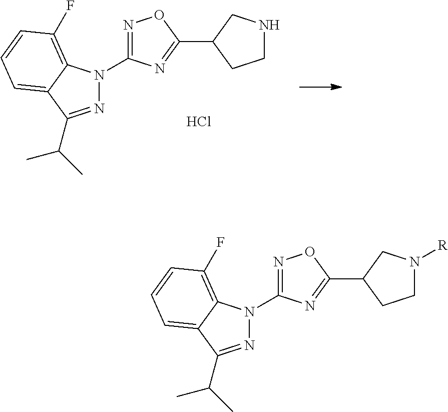

Wherein the structure of R is defined in the following table.

TABLE 69

| | | | ¹H-NMR/ |
|---|---|---|---|
| Ex. | R | Compound Name | LC-MS, m/z |
| 455 | 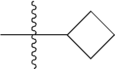 [SMILES: *C1CCC1] | 1-[5-(1-cyclobutyl- pyrrolidin-3-yl)- 1,2,4-oxadiazol-3-yl]- 7-fluoro-3-(propan-2- yl)-1H-indazole | LC-MS, m/z; 370 [M + H]+ |
| 456 | 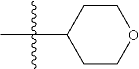 [SMILES: *C1CCOCC1] | 7-fluoro-3-(propan-2- yl)-1-{5-[1-(tetrahydro- 2H-pyran-4-yl)pyrrolidin- 3-yl]-1,2,4-oxadiazol- 3-yl}-1H-indazole | LC-MS, m/z; 400 [M + H]+ |

### Example 457

### Preparation of 1-(4-{3-[7-fluoro-3-(propan-2-yl)-1H-indazol-1-yl]-1,2,4-oxadiazol-5-yl}-4-hydroxy-1,4′-bipiperidin-1′-yl)ethanone

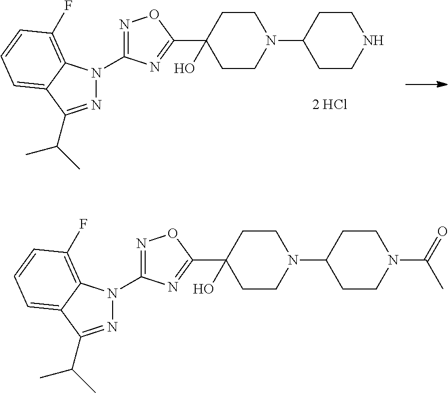 [SMILES: CC(C)c1nn(-c2noc(C3(O)CCN(C4CCNCC4)CC3)n2)c2c(F)cccc12 ; "2 HCl" ; CC(=O)N1CCC(N2CCC(O)(c3nc(-n4nc(C(C)C)c5cccc(F)c54)no3)CC2)CC1]

The title compound was prepared in the same manner as in Example 168 except that the 3-ethyl-1-(5-{1-[2-(piperidin-4-yl)ethyl]piperidin-4-yl}-1,2,4-oxadiazol-3-yl)-1H-indazole bis(trifluoroacetate) and methyl chloroformate were replaced with 4-{3-[7-fluoro-3-(propan-2-yl)-1H-indazol-1-yl]-1,2,4-oxadiazol-5-yl}-1,4′-bipiperidin-4-ol dihydrochloride and acetyl chloride, respectively.

LC-MS, m/z; 471 [M+H]+

### Preparations of Examples 458 to 466

The compounds in the following table (i.e. Examples 458 to 466) were prepared in the same manner as in Example 168 except that the 3-ethyl-1-(5-{1-[2-(piperidin-4-yl)ethyl]piperidin-4-yl}-1,2,4-oxadiazol-3-yl)-1H-indazole bis(trifluoroacetate) and methyl chloroformate were replaced with the corresponding starting compound and acid chloride (defined as R—Cl) or acetic anhydride, respectively.

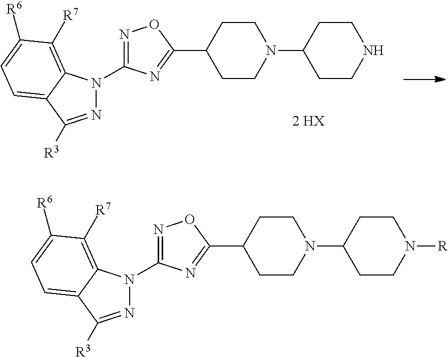

Wherein HX is hydrochloric acid or trifluoroacetic acid, and the structure of R is defined in the following table. Each free form of the compounds in the following table was obtained by omitting the conversion step into hydrochloride in Example 168. In order to obtain each of the trifluoroacetates, the residue was isolated/purified by reverse phase HPLC.

TABLE 70

| Ex. | R³ | R⁶ | R⁷ | R | Compound Name | ¹H-NMR/LC-MS, m/z |
|---|---|---|---|---|---|---|
| 458 | ⁱPr | H | H | —Ac | 1-(4-{3-[3- | LC-MS, m/z; |
| | | | | | (propan-2- | 437 [M + H]+ |
| | | | | | yl)-1H- | |
| | | | | | indazol-1- | |
| | | | | | yl]-1,2,4- | |
| | | | | | oxadiazol-5- | |
| | | | | | yl}-1,4′- | |
| | | | | | bipiperidin- | |
| | | | | | 1′- | |
| | | | | | yl)ethanone | |
| | | | | | trifluoro- | |
| | | | | | acetate | |
| 459 | Et | F | F | —CO₂Me | methyl 4-[3- | ¹H-NMR (CDCl₃) δ: |
| | | | | | (3-ethyl-6,7- | 1.45 (3H, t, J = 7.4 |
| | | | | | difluoro-1H- | Hz), 1.62-1.80 (2H, |
| | | | | | indazol-1- | m), 2.03-2.19 (2H, |
| | | | | | yl)-1,2,4- | m), 2.32-3.00 (7H, |
| | | | | | oxadiazol-5- | m), 3.00-3.11 (2H, |
| | | | | | yl]-1,4′- | m), 3.11-3.60 (4H, |
| | | | | | bipiperidine- | m), 3.63-3.81 (4H, |
| | | | | | 1′- | m), 4.18-4.53 (2H, |
| | | | | | carboxylate | m), 7.13-7.31 (1H, |
| | | | | | trifluoro- | m), 7.40-7.59 (1H, |
| | | | | | acetate | m). |
| | | | | | | LC-MS, m/z; 475 |
| | | | | | | [M + H]+ |
| 460 | Et | F | F | —Ac | 1-{4-[3-(3- | ¹H-NMR (CDCl₃) δ: |
| | | | | | ethyl-6,7- | 1.44 (3H, t, J = 7.4 |
| | | | | | difluoro-1H- | Hz), 1.52-1.88 (2H, |
| | | | | | indazol-1- | m), 2.14 (3H, s), |
| | | | | | yl)-1,2,4- | 2.20-2.76 (7H, m), |
| | | | | | oxadiazol-5- | 2.85-3.90 (9H, m), |
| | | | | | yl]-1,4′- | 3.90-4.10 (1H, m), |
| | | | | | bipiperidin- | 4.75-4.96 (1H, m), |
| | | | | | 1′- | 7.10-7.30 (1H, m), |
| | | | | | yl}ethanone | 7.40-7.60 (1H, m), |
| | | | | | trifluoro- | LC-MS, m/z; 481 |
| | | | | | acetate | [M + Na]+ |
| 461 | Et | F | F | 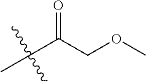 [SMILES: *C(=O)COC] | 1-{4-[3-(3- ethyl-6,7- difluoro-1H- indazol-1- yl)-1,2,4- oxadiazol-5- yl]-1,4′- bipiperidin- 1′-yl}-2- methoxy- ethanone trifluoro- acetate | ¹H-NMR (CDCl₃) δ: 1.46 (3H, t, J = 7.6 Hz), 1.60-1.83 (2H, m), 2.09-2.30 (2H, m), 2.35-2.80 (5H, m), 3.00-3.15 (3H, m), 3.20-3.82 (9H, m), 4.00-4.23 (3H, m), 4.73-4.92 (1H, m), 7.15-7.33 (1H, m), 7.42-7.58 (1H, m). LC-MS, m/z; 489 [M + H]+ |
| 462 | ⁱPr | Me | H | —Ac | 1-(4-{3-[6- | LC-MS, m/z; |
| | | | | | methyl-3- | 451 [M + H]+ |
| | | | | | (propan-2- | |
| | | | | | yl)-1H- | |
| | | | | | indazol-1- | |
| | | | | | yl]-1,2,4- | |
| | | | | | oxadiazol-5- | |
| | | | | | yl}-1,4′- | |
| | | | | | bipiperidin- | |
| | | | | | 1′- | |
| | | | | | yl}ethanone | |
| | | | | | trifluoro- | |
| | | | | | acetate | |
| 463 | ⁱPr | F | F | —CO₂Me | methyl 4-{3- | LC-MS, m/z; |
| | | | | | [6,7- | 489 [M + H]+ |
| | | | | | difluoro-3- | |
| | | | | | (propan-2- | |
| | | | | | yl)-1H- | |
| | | | | | indazol-1- | |
| | | | | | yl]-1,2,4- | |
| | | | | | oxadiazol-5- | |
| | | | | | yl}-1,4′- | |
| | | | | | bipiperidine | |
| | | | | | 1′- | |
| | | | | | carboxylate | |
| | | | | | trifluoro- | |
| | | | | | acetate | |
| 464 | ⁱPr | F | F | —Ac | 1-(4-{3-[6, | ¹H-NMR (CD₃OD) δ: |
| | | | | | 7-difluoro-3- | 1.48 (6H, d, J = |
| | | | | | (propan-2- | 7.2 Hz), 1.60-1.89 |
| | | | | | yl)-1H- | (2H, m), 2.10-2.35 |
| | | | | | indazol-1- | (7H, m), 2.35-2.78 |
| | | | | | yl]-1,2,4- | (3H, m), 3.12-3.30 |
| | | | | | oxadiazol-5- | (2H, m), 3.40-3.69 |
| | | | | | yl}-1,4′- | (4H, m), 3.69-3.85 |
| | | | | | bipiperidin- | (2H, m), 4.04-4.20 |
| | | | | | 1′- | (1H, m), 4.65-4.80 |
| | | | | | yl}ethanone | (1H, m), 7.25-7.40 |
| | | | | | trifluoro- | (1H, m), 7.69-7.80 |
| | | | | | acetate | (1H, m). |
| 465 | ⁱPr | F | F | 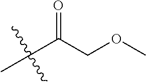 [SMILES: *C(=O)COC] | 1-(4-{3-[6, 7-difluoro-3- (propan-2- yl)-1H- indazol-1- yl]-1,2,4- oxadiazol-5- yl}-1,4′- bipiperidin- 1′-yl)-2- methoxy- ethanone trifluoro- acetate | ¹H-NMR (CDCl₃) δ: 1.48 (6H, t, J = 7.2 Hz), 1.54-1.82 (2H, m), 2.04-2.27 (2H, m), 2.27-2.79 (5H, m), 2.98-3.15 (1H, m), 3.15-3.80 (10H, m), 3.98-4.20 (3H, m), 4.70-4.88 (1H, m), 7.10-7.25 (1H, m), 7.47-7.60 (1H, m). LC-MS, m/z; 525 [M + Na]+ |
| 466 | ⁱPr | F | F | -Ms | 4-{3-[7- | ¹H-NMR (CDCl₃) δ: |
| | | | | | fluoro-3- | 1.50 (6H, d, J = 7.0 |
| | | | | | (propan-2- | Hz), 1.63-1.77 (2H, |
| | | | | | yl)-1H- | m), 1.84-1.96 (2H, |
| | | | | | indazol-1- | m), 1.99-2.11 (2H, |
| | | | | | yl]-1,2,4- | m), 2.15-2.25 (2H, |
| | | | | | oxadiazol-5- | m), 2.33-2.51 (3H, |
| | | | | | yl}-1′- | m), 2.66-2.77 (2H, |
| | | | | | (methyl- | m), 2.79 (3H, s), |
| | | | | | sulfonyl)-1,4′- | 2.93-3.12 (3H, m), |
| | | | | | bipiperidine | 3.43-3.53 (1H, m), |
| | | | | | trifluoro- | 3.85 (2H, d, J = 12.3 |
| | | | | | acetate | Hz), 7.18-7.29 (2H, |
| | | | | | | m), 7.56-7.63 (1H, |
| | | | | | | m). |
| | | | | | | LC-MS, m/z; 491 |
| | | | | | | [M + H]+ |

### Preparations of Examples 467 to 494

The compounds in the following table (i.e. Examples 467 to 494) were prepared in the same manner as in Example 168 except that the 3-ethyl-1-(5-{1-[2-(piperidin-4-yl)ethyl]piperidin-4-yl}-1,2,4-oxadiazol-3-yl)-1H-indazole bis(trifluoroacetate) and methyl chloroformate were replaced with the corresponding starting compound and acid chloride (defined as R—Cl) or acetic anhydride, respectively.

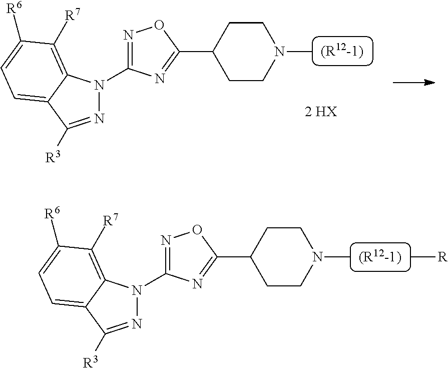

Wherein HX is hydrochloric acid or trifluoroacetic acid, (R¹²-1) means each cyclic amino structure shown in the following table, and the structure of R is defined in the following table. R is attached to the nitrogen atom in the cyclic amine of (R¹²-1). Each free form of the compounds in the following table was obtained by omitting the conversion step into hydrochloride in Example 168. In order to obtain each of the trifluoroacetates, the residue was isolated/purified by reverse phase HPLC.

TABLE 71

| | | | | | | Compound | ¹H-NMR/ |
|---|---|---|---|---|---|---|---|
| Ex. | R³ | R⁶ | R⁷ | (R¹²-1) | R | Name | LC-MS, m/z |
| 467 | ⁱPr | H | H | 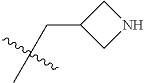 [SMILES: *CC1CNC1] | —Ac | 1-{3-[(4- {3-[3- (propan-2- yl)-1H- indazol-1- yl]-1,2,4- oxadiazol- 5- yl}piper- idin-1- yl)methyl] azetidin-1- yl}ethanone trifluoro- acetate | LC-MS, m/z; 445 [M + Na]+ |
| 468 469 | Et Et | F F | F F | 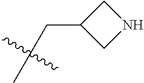 [SMILES: *CC1CNC1] | —Ac 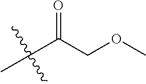 [SMILES: *C(=O)COC] | 1-[3-({4- [3-(3- ethyl-6,7- difluoro- 1H-indazol- 1-yl)- 1,2,4- oxadiazol- 5- yl]piper- idin-1- yl}methyl) azetidin-1- yl]ethanone trifluoro- acetate 1-[3-({4- [3-(3- ethyl-6,7- difluoro- 1H-indazol- 1-yl)- 1,2,4- oxadiazol- 5- yl]piper- idin-1- yl}methyl) azetidin-1- yl]-2- methoxy- ethanone trifluoro- acetate | ¹H-NMR (CDCl₃) δ: 1.43 (3H, t, J = 7.6 Hz), 1.86 (3H, s), 2.30-2.68 (4H, m), 3.04 (2H, q, J = 7.6 Hz), 3.10- 3.90 (9H, m), 3.93- 4.03 (1H, m), 4.18- 4.30 (1H, m), 4.30- 4.43 (1H, m), 7.12- 7.26 (1H, m), 7.40- 7.55 (1H, m). LC-MS, m/z; 445 [M + H]+ LC-MS, m/z; 475 [M + H]+ |
| 470 471 472 | Et ⁱPr ⁱPr | F Me F | F H F | 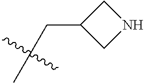 [SMILES: *CC1CNC1] | —CO₂Me —Ac —Ac | methyl 3- ({4-[3-(3- ethyl-6,7- difluoro- 1H-indazol- 1-yl)- 1,2,4- oxadiazol- 5- yl]piper- idin-1- yl}methyl) azetidine-1- carboxylate trifluoro- acetate 1-{3-[(4- {3-[6- methyl-3- (propan-2- yl)-1H- indazol-1- yl]-1,2,4- oxadiazol- 5- yl}piper- idin-1- yl)methyl] azetidin-1- yl}ethanone trifluoro- acetate 1-{3-[(4- {3-[6,7- difluoro-3- (propan-2- yl)-1H- indazol-1- yl]-1,2,4- oxadiazol- 5- yl}piper- idin-1- yl)methyl] azetidin-1- yl}ethanone trifluoro- acetate | LC-MS, m/z; 461 [M + H]+ LC-MS, m/z; 437 [M + H]+ ¹H-NMR (CD₃OD) δ: 1.46 (6H, d, J = 7.2 Hz), 1.87 (3H, s), 2.18-2.59 (4H, m), 3.15-3.37 (3H, m), 3.41-3.84 (7H, m), 4.02-4.1 (1H, m), , 4.16-4.28 (1H, m), 4.40-4.45 (1H, m), 7.25-7.40 (1H, m), 7.69-7.80 (1H, m). |
| 473 474 | ⁱPr ⁱPr | F F | F F | 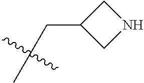 [SMILES: *CC1CNC1] | 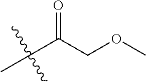 [SMILES: *C(=O)COC] —CO₂Me | 1-{3-[(4- {3-[6,7- difluoro-3- (propan-2- yl)-1H- indazol-1- yl]-1,2,4- oxadiazol- 5- yl}piper- idin-1- yl)methyl] azetidin-1- yl}-2- methoxy- ethanone trifluoro- acetate methyl 3- [(4-{3-[6, 7-difluoro- 3-(propan- 2-yl)-1H- indazol-1- yl]-1,2,4- oxadiazol- 5- yl}piper- idin-1- yl)methyl] azetidine-1- carboxylate trifluoro- acetate | ¹H-NMR (CD₃OD) δ: 1.46 (6H, d, J = 7.2 Hz), 2.18- 2.59 (4H, m), 3.19- 3.37 (3H, m), 3.38 (3H, s), 3.41-3.84 (7H, m), 4.02-4.1 (1H, m), , 4.16-4.28 (1H, m), 4.40-4.5 (1H, m), 7.25-7.40 (1H, m), 7.69-7.80 (1H, m). ¹H-NMR (CD₃OD) δ: 1.46 (6H, d, J = 7.2 Hz), 2.18- 2.59 (4H, m), 3.15- 3.37 (3H, m), 3.4- 3.6 (4H, m), 3.61- 3.9 (7H, m), 4.15- 4.38 (2H, m), 7.25- 7.40 (1H, m), 7.69- 7.80 (1H, m). |
| 475 | ⁱPr | H | H | 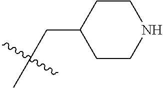 [SMILES: *CC1CCNCC1] | —Ac | 1-{4-[(4- {3-[3- (propan-2- yl)-1H- indazol-1- yl]-1,2,4- oxadiazol 5- yl}piper- idin-1- yl)methyl] piperidin-1- yl}ethanone trifluoro- acetate | LC-MS, m/z; 451 [M + H]+ |
| 476 477 | Et Et | F F | F F | 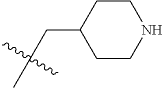 [SMILES: *CC1CCNCC1] | —CO₂Me 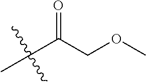 [SMILES: *C(=O)COC] | methyl 4- ({4-[3-(3- ethyl-6,7- difluoro- 1H-indazol- 1-yl)- 1,2,4- oxadiazol- 5- yl]piper- idin-1- yl}methyl) piperidin- 1- carboxylate trifluoro- acetate 1-[4-({4- [3-(3- ethyl-6,7- difluoro- 1H-indazol- 1-yl)- 1,2,4- oxadiazol- 5- yl]piper- idin-1- yl}methyl) piperidin-1- yl]-2- methoxy- ethanone trifluoro- acetate | ¹H-NMR (CDCl₃) δ: 1.00-1.18 (2H, m), 1.42 (3H, t, J = 7.6 Hz), 1.55- 1.85 (4H, m), 1.94- 2.30 (8H, m), 2.68- 2.81 (2H, m), 2.82- 3.00 (2H, m), 3.04 (2H, q, J = 7.6 Hz), 3.68 (3H, s), 3.95- 4.30 (2H, br), 7.10- 7.21 (1H, m), 7.40- 7.50 (1H, m). LC-MS, m/z; 489 [M + H]+ ¹H-NMR (CDCl₃) δ: 1.17-1.38 (2H, m), 1.43 (3H, t, J = 7.6 Hz), 1.76- 2.25 (3H, m), 2.30- 2.80 (5H, m), 2.80- 3.38 (7H, m), 3.40 (3H, s), 3.50-3.92 (4H, m), 4.00-4.17 (2H, m), 4.50-4.69 (1H, m), 7.10-7.25 (1H, m), 7.40-7.56 (1H, m). LC-MS, m/z; 503 [M + H]+ |
| 478 | Et | F | F | 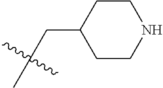 [SMILES: *CC1CCNCC1] | —Ac | 1-[4-({4- [3-(3- ethyl-6,7- difluoro- 1H-indazol- 1-yl)- 1,2,4- oxadiazol- 5- yl]piper- idin-1- yl}methyl) piperidin-1- yl]ethanone trifluoro- acetate | LC-MS, m/z; 473 [M + H]+ |
| 479 480 | ⁱPr ⁱPr | Me F | H F | 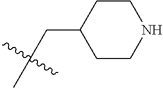 [SMILES: *CC1CCNCC1] | —Ac —Ac | 1-{4-[(4- {3-[6- methyl-3- (propan-2- yl)-1H- indazol-1- yl]-1,2,4- oxadiazol- 5- yl}piper- idin-1- yl)methyl] piperidin-1- yl}ethanone trifluoro- acetate 1-{4-[(4- {3-[6,7- difluoro-3- (propan-2- yl)-1H- indazol-1- yl]-1,2,4- oxadiazol- 5- yl}piper- idin-1- yl)methyl] piperidin-1- yl}ethanone trifluoro- acetate | LC-MS, m/z; 487 [M + Na]+ LC-MS, m/z; 487 [M + H]+ |
| 481 | ⁱPr | F | F | 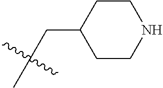 [SMILES: *CC1CCNCC1] | 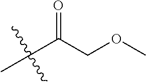 [SMILES: *C(=O)COC] | 1-{4-[(4- {3-[6,7- difluoro-3- (propan-2- yl)-1H- indazol-1- yl]-1,2,4- oxadiazol- 5- yl}piper- idin-1- yl)methyl] piperidin-1- yl}-2- methoxy- ethanone trifluoro- acetate | ¹H-NMR (CDCl₃) δ: 1.12-1.35 (2H, m), 1.35-1.54 (6H, m), 1.77-2.03 (2H, m), 2.07-2.25 (1H, m), 2.31-2.71 (5H, m), 2.90-3.14 (4H, m), 3.17-3.50 (2H, m), 3.38 (3H, s), 3.60- 3.90 (4H, m), 4.00- 4.20 (2H, m), 4.45- 4.64 (1H, m), 7.09- 7.21 (1H, m), 7.41- 7.58 (1H, m). LC-MS, m/z; 539 [M + Na]+ |
| 482 | ⁱPr | F | F | 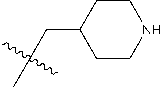 [SMILES: *CC1CCNCC1] | —CO₂Me | methyl 4- [(4-{3- [6,7- difluoro-3- (propan-2- yl)-1H- indazol-1- yl]-1,2,4- oxadiazol- 5- yl}piper- idin-1- yl)methyl] piperidine- 1- carboxylate trifluoro- acetate | LC-MS, m/z; 525 [M + Na]+ |
| 483 484 | ⁱPr ⁱPr | H H | F F | 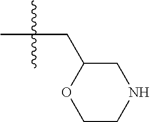 [SMILES: *CC1CNCCO1] | —Ac —CO₂Me | 1-{2-[(4- {3-[7- fluoro-3- (propan-2- yl)-1H- indazol-1- yl]-1,2,4- oxadiazol- 5- yl}piper- idin-1- yl)methyl] morpholin-4- yl}ethanone methyl 2- [(4-{3-[7- fluoro-3- (propan-2- yl)-1H- indazol-1- yl]-1,2,4- oxadiazol- 5- yl}piper- idin-1- yl)methyl] morpholine- 4- carboxylate | LC-MS, m/z; 471 [M + H]+ LC-MS, m/z; 487 [M + H]+ |
| 485 486 | ⁱPr ⁱPr | H H | F F | 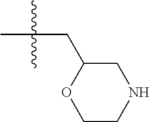 [SMILES: *CC1CNCCO1] | 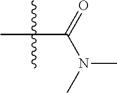 [SMILES: *C(=O)N(C)C] -Ms | 2-[(4-{3- [7-fluoro- 3-(propan- 2-yl)-1H- indazol-1- yl]-1,2,4- oxadiazol- 5- yl}piper- idin-1- yl)methyl]- N,N- dimethyl- morpholine-4- carboxamide 7-fluoro-1- [5-(1-{[4- (methyl- sulfonyl)mor- pholin-2- yl]methyl} piperidin-4- yl)-1,2,4- oxadiazol- 3-yl]-3- (propan-2- yl)-1H- indazole | LC-MS, m/z; 500 [M + H]+ LC-MS, m/z; 507 [M + H]+ |
| 487 | ⁱPr | H | F | 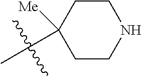 [SMILES: *C1(C)CCNCC1] | —Ac | 1-(4-{3-[7- fluoro-3- (propan-2- yl)-1H- indazol-1- yl]-1,2,4- oxadiazol- 5-yl}-4′- methyl- 1,4′- bipiper- idin-1′- yl)ethanone | ¹H-NMR (CDCl₃) δ: 0.95 (3H, s), 1.24- 1.47 (2H, m), 1.50 (6H, dd, J = 7.0, 0.6 Hz), 1.82- 2.38 (11H, m), 2.92- 3.11 (3H, m), 3.19- 3.59 (4H, m), 3.86- 3.99 (1H, m), 7.16- 7.29 (2H, m), 7.55- 7.63 (1H, m). LC-MS, m/z; 469 [M + H]+ |
| 488 | ⁱPr | H | F | 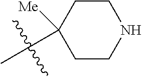 [SMILES: *C1(C)CCNCC1] | -Ms | 4-{3-[7- fluoro-3- (propan-2- yl)-1H- indazol-1- yl]-1,2,4- oxadiazol- 5-yl}-4′- methyl-1′- (methyl- sulfonyl)- 1,4′- bipiperidine | ¹H-NMR (CDCl₃) δ: 0.96 (3H, s), 1.47- 1.61 (8H, m), 1.90- 2.08 (4H, m), 2.15- 2.35 (4H, m), 2.80 (3H, s), 2.94-3.16 (5H, m), 3.36-3.55 (3H, m), 7.18-7.29 (2H, m), 7.56-7.62 (1H, m). LC-MS, m/z; 505 [M + H]+ |
| 489 | ⁱPr | H | F | 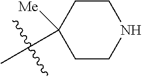 [SMILES: *C1(C)CCNCC1] | 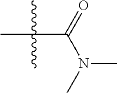 [SMILES: *C(=O)N(C)C] | 4-{3-[7- fluoro-3- (propan-2- yl)-1H- indazol-1- yl]-1,2,4- oxadiazol- 5-yl}- N,N,4′- trimethyl- 1,4′- bipiper- idine-1′- carboxamide | ¹H-NMR (CDCl₃) δ: 0.94 (3H, s), 1.39- 1.57 (8H, m), 1.77- 1.87 (2H, m), 1.92- 2.08 (2H, m), 2.11- 2.35 (4H, m), 2.81 (6H, s), 2.97-3.10 (3H, m), 3.15-3.37 (4H, m), 3.44-3.53 (1H, m), 7.17-7.29 (2H, m), 7.55-7.63 (1H, m). LC-MS, m/z; 498 [M + H]+ |
| 490 | ⁱPr | H | F | 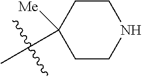 [SMILES: *C1(C)CCNCC1] | 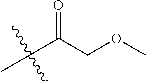 [SMILES: *C(=O)COC] | 1-(4-{3-[7- fluoro-3- (propan-2- yl)-1H- indazol-1- yl]-1,2,4- oxadiazol- 5-yl}-4′- methyl- 1,4′- bipiper- idin-1′-yl)-2- methoxy- ethanone | ¹H-NMR (CDCl₃) δ: 0.93 (3H, s), 1.28- 1.43 (2H, m), 1.47 (6H, d, J = 7.0 Hz), 1.83-2.07 (4H, m), 2.10-2.36 (4H, m), 2.90-3.09 (3H, m), 3.17-3.32 (1H, m), 3.36-3.53 (6H, m), 3.88-3.99 (1H, m), 4.08 (2H, dd, J = 17.5, 13.3 Hz), 7.15- 7.25 (2H, m), 7.53- 7.60 (1H, m). LC-MS, m/z; 499 [M + H]+ |
| 491 | Et | H | F | 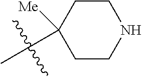 [SMILES: *C1(C)CCNCC1] | —Ac | 1-{4-[3-(3- ethyl-7- fluoro-1H- indazol-1- yl)-1,2,4- oxadiazol- 5-yl]-4′- methyl- 1,4′- bipiper- idin-1′- yl}ethanone | ¹H-NMR (CDCl₃) δ: 0.93 (3H, s), 1.25- 1.46 (5H, m), 1.81- 2.08 (7H, m), 2.11- 2.34 (4H, m), 2.91- 3.10 (5H, m), 3.14- 3.55 (3H, m), 3.84- 3.99 (1H, m), 7.17- 7.27 (2H, m), 7.48- 7.53 (1H, m). LC-MS, m/z; 455 [M + H]+ |
| 492 493 | Et Et | H H | F F | 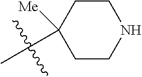 [SMILES: *C1(C)CCNCC1] | -Ms 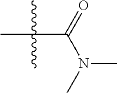 [SMILES: *C(=O)N(C)C] | 4-[3-(3- ethyl-7- fluoro-1H- indazol-1- yl)-1,2,4- oxadiazol- 5-yl]-4′- methyl-1′- (methyl- sulfonyl)-1,4′- bipiperidine 4-[3-(3- ethyl-7- fluoro-1H- indazol-1- yl)-1,2,4- oxadiazol- 5-yl]- N,N,4′- trimethyl- 1,4′- bipiper- idine-1′- carboxamide | ¹H-NMR (CDCl₃) δ: 0.93 (3H, s), 1.38- 1.53 (5H, m), 1.89- 2.05 (4H, m), 2.13- 2.31 (4H, m), 2.78 (3H, s), 2.89-3.13 (7H, m), 3.33-3.45 (2H, m), 7.18-7.29 (2H, m), 7.49-7.54 (1H, m). LC-MS, m/z; 491 [M + H]+ ¹H-NMR (CDCl₃) δ: 0.91 (3H, s), 1.31- 1.51 (5H, m), 1.73- 1.85 (2H, m), 1.89- 2.05 (2H, m), 2.08- 2.32 (4H, m), 2.79 (6H, s), 2.92-3.10 (5H, m), 3.12-3.34 (4H, m), 7.18-7.26 (2H, m), 7.48-7.53 (1H, m). LC-MS, m/z; 483 [M + H]+ |
| 494 | Et | H | F | 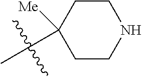 [SMILES: *C1(C)CCNCC1] | 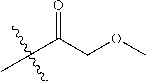 [SMILES: *C(=O)COC] | 1-{4-[3-(3- ethyl-7- fluoro-1H- indazol-1- yl)-1,2,4- oxadiazol- 5-yl]-4′- methyl- 1,4′- bipiper- idin-1′-yl}-2- methoxy- ethanone | ¹H-NMR (CDCl₃) δ: 0.93 (3H, s), 1.28- 1.47 (5H, m), 1.81- 2.07 (4H, m), 2.12- 2.35 (4H, m), 2.91- 3.11 (5H, m), 3.17- 3.31 (1H, m), 3.34- 3.52 (5H, m), 3.88- 4.00 (1H, m), 4.00- 4.17 (2H, m), 7.16- 7.27 (2H, m), 7.48- 7.54 (1H, m). LC-MS, m/z; 485 [M + H]+ |

### Preparations of Examples 495 to 506

The compounds in the following table (i.e. Examples 495 to 506) were prepared in the same manner as in Example 168 except that the 3-ethyl-1-(5-{1-[2-(piperidin-4-yl)ethyl]piperidin-4-yl}-1,2,4-oxadiazol-3-yl)-1H-indazole bis(trifluoroacetate) and methyl chloroformate were replaced with the corresponding starting compound and acid chloride (defined as R—Cl) or acetic anhydride, respectively.

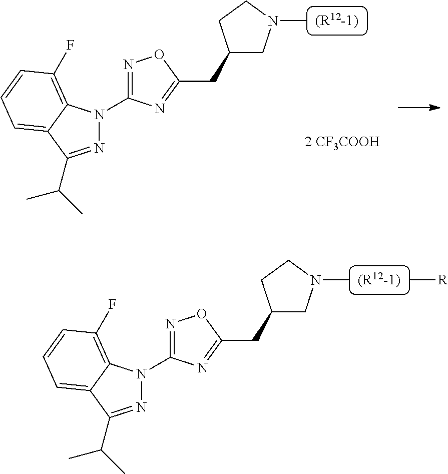

Wherein (R¹²-1) means each cyclic amino structure shown in the following table, and the structure of R is defined in the following table. R is attached to the nitrogen atom in the cyclic amine of (R¹²-1). Each free form of the compounds in the following table was obtained by omitting the conversion step into hydrochloride in Example 168.

TABLE 72

| Ex. | (R¹²-1) | R | Compound Name | ¹H-NMR/LC-MS, m/z |
|---|---|---|---|---|
| 495 | 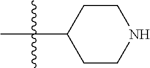 [SMILES: *C1CCNCC1] | —Ac | 1-{4-[(3R)-3-({3- [7-fluoro-3- (propan-2-yl)-1H- indazol-1-yl]- 1,2,4-oxadiazol-5- yl}methyl)pyrrolidin- 1-yl]piperidin-1- yl}ethanone | ¹H-NMR (CDCl₃) δ: 1.39-1.99 (12H, m), 2.08 (3H, s), 2.12- 3.17 (10H, m), 3.41- 3.55 (1H, m), 3.78 (1H, d, J = 13.6 Hz), 4.43 (1H, d, J = 10.5 Hz), 7.18-7.30 (2H, m), 7.56-7.63 (1H, m). LC-MS, m/z; 455 [M + H]+ |
| 496 | | —Ms | 7-fluoro-1-[5- ({(3R)-1-[1- (methylsulfonyl) piperidin-4- yl]pyrrolidin-3- yl}methyl)-1,2,4- oxadiazol-3-yl]-3- (propan-2-yl)-1H- indazole | ¹H-NMR (CDCl₃) δ: 1.50 (6H, d, J = 7.0 Hz), 1.59-1.74 (3H, m), 1.87-1.99 (2H, m), 2.09-2.27 (2H, m), 2.42-2.51 (1H, m), 2.57-2.96 (9H, m), 3.08 (2H, d, J = 6.8 Hz), 3.42-3.54 (1H, m), 3.56-3.68 (2H, m), 7.18-7.30 (2H, m), 7.56-7.63 (1H, m). LC-MS, m/z; 491 [M + H]+ |
| 497 | 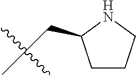 [SMILES: *C[C@@H]1CCCN1] | —Ac | 1-[(2S)-2-{[(3R)-3- ({3-[7-fluoro-3- (propan-2-yl)-1H- indazol-1-yl]- 1,2,4-oxadiazol-5- yl}methyl)pyrrolidin- 1-yl]methyl}pyrrolidin- 1-yl]ethanone | ¹H-NMR (CDCl₃) δ: 1.50 (6H, d, J = 7.0 Hz), 1.55-1.71 (1H, m), 1.80-2.20 (8H, m), 2.32-2.88 (7H, m), 3.01-3.11 (2H, m), 3.31-3.57 (3H, m), 3.82-3.93 (0.4H, m, rotamer), 4.14- 4.27 (0.6H, m, rotamer), 7.18-7.27 (2H, m), 7.55-7.63 (1H, m). LC-MS, m/z; 455 [M + H]+ |
| 498 | | —Ms | 7-fluoro-1-(5- {[(3R)-1-{[(2S)-1- (methylsulfonyl) pyrrolidin-2- yl]methyl}pyrrolidin- 3-yl]methyl}- 1,2,4-oxadiazol-3- yl)-3-(propan-2- yl)-1H-indazole | ¹H-NMR (CDCl₃) δ: 1.50 (6H, d, J = 7.1 Hz), 1.57-1.67 (1H, m), 1.84-2.00 (4H, m), 2.06-2.17 (1H, m), 2.46-2.68 (4H, m), 2.72-2.83 (3H, m), 2.88 (3H, s), 3.02-3.08 (2H, m), 3.29-3.41 (2H, m), 3.44-3.53 (1H, m), 3.75-3.85 (1H, m), 7.19-7.27 (2H, m), 7.56-7.62 (1H, m). LC-MS, m/z; 491 [M + H]+ |
| 499 | 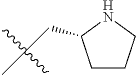 [SMILES: *C[C@H]1CCCN1] | —Ac | 1-[(2R)-2-{[(3R)-3- ({3-[7-fluoro-3- (propan-2-yl)-1H- indazol-1-yl]- 1,2,4-oxadiazol-5- yl}methyl)pyrrolidin- 1-yl]methyl}pyrrolidin- 1-yl]ethanone | ¹H-NMR (CDCl₃) δ: 1.50 (6H, d, J = 7.0 Hz), 1.55-1.70 (1H, m), 1.77-2.20 (8H, m), 2.32-2.89 (7H, m), 2.98-3.14 (2H, m), 3.31-3.56 (3H, m), 3.80-3.95 (0.4H, m, rotamer), 4.13- 4.26 (0.6H, m, rotamer), 7.18-7.29 (2H, m), 7.55-7.63 (1H, m). LC-MS, m/z; 455 [M + H]+ |
| 500 | 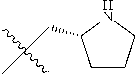 [SMILES: *C[C@H]1CCCN1] | —Ms | 7-fluoro-1-(5- {[(3R)-1-{[(2R)-1- (methylsulfonyl) pyrrolidin-2- yl]methyl}pyrrolidin- 3-yl]methyl}- 1,2,4-oxadiazol-3- yl)-3-(propan-2- yl)-1H-indazole | ¹H-NMR (CDCl₃) δ: 1.50 (6H, d, J = 7.0 Hz), 1.54-1.69 (1H, m), 1.82-2.00 (4H, m), 2.05-2.21 (1H, m), 2.43-2.86 (7H, m), 2.88 (3H, s), 3.02-3.10 (2H, m), 3.29-3.41 (2H, m), 3.43-3.54 (1H, m), 3.75-3.87 (1H, m), 7.18-7.27 (2H, m), 7.56-7.63 (1H, m). LC-MS, m/z; 491 [M + H]+ |
| 501 | 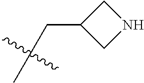 [SMILES: *CC1CNC1] | —Ac | 1-(3-{[(3R)-3-({3- [7-fluoro-3- (propan-2-yl)-1H- indazol-1-yl]- 1,2,4-oxadiazol-5- yl}methyl)pyrrolidin- 1-yl]methyl}azetidin- 1-yl)ethanone | ¹H-NMR (CDCl₃) δ: 1.50 (6H, d, J = 7.2 Hz), 1.56-1.72 (1H, m), 1.83 (3H, d, J = 1.7 Hz), 2.06-2.24 (1H, m), 2.37-2.85 (8H, m), 3.06 (2H, d, J = 6.4 Hz), 3.41- 3.55 (1H, m), 3.59- 3.68 (1H, m), 3.75- 3.82 (1H, m), 4.02- 4.23 (2H, m), 7.18- 7.30 (2H, m), 7.55- 7.63 (1H, m). LC-MS, m/z; 441 [M + H]+ |
| 502 | | —Ms | 7-fluoro-1-(5- {[(3R)-1-{[1- (methylsulfonyl) azetidin-3- yl]methyl}pyrrolidin- 3-yl]methyl}- 1,2,4-oxadiazol-3- yl)-3-(propan-2- yl)-1H-indazole | ¹H-NMR (CDCl₃) δ: 1.50 (6H, d, J = 7.0 Hz), 1.57-1.71 (1H, m), 2.07-2.22 (1H, m), 2.36-2.82 (8H, m), 2.84 (3H, s), 3.05 (2H, d, J = 7.2 Hz), 3.41-3.54 (1H, m), 3.66 (2H, dd, J = 7.8, 5.8 Hz), 3.93- 4.03 (2H, m), 7.19- 7.29 (2H, m), 7.56- 7.63 (1H, m). LC-MS, m/z; 477 [M + H]+ |
| 503 | 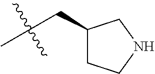 [SMILES: *C[C@@H]1CCNC1] | —Ac | 1-[(3S)-3-{[(3R)-3- ({3-[7-fluoro-3- (propan-2-yl)-1H- indazol-1-yl]- 1,2,4-oxadiazol-5- yl}methyl)pyrrolidin- 1-yl]methyl}pyrrolidin- 1-yl]ethanone | ¹H-NMR (CDCl₃) δ: 1.44-1.80 (8H, m), 1.95-2.23 (5H, m), 2.29-2.86 (8H, m), 3.02-3.21 (3H, m), 3.31-3.74 (4H, m), 7.18-7.28 (2H, m), 7.56-7.62 (1H, m). LC-MS, m/z; 455 [M + H]+ |
| 504 | 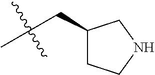 [SMILES: *C[C@@H]1CCNC1] | —Ms | 7-fluoro-1-(5- {[(3R)-1-{[(3S)-1- (methylsulfonyl) pyrrolidin-3- yl]methyl}pyrrolidin- 3-yl]methyl}- 1,2,4-oxadiazol-3- yl)-3-(propan-2- yl)-1H-indazole | ¹H-NMR (CDCl₃) δ: 1.50 (6H, d, J = 7.0 Hz), 1.55-1.77 (2H, m), 1.99-2.24 (2H, m), 2.36-2.80 (8H, m), 2.82 (3H, s), 3.01-3.13 (3H, m), 3.24-3.56 (4H, m), 7.18-7.30 (2H, m), 7.56-7.63 (1H, m). LC-MS, m/z; 491 [M + H]+ |
| 505 | 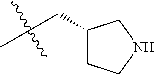 [SMILES: *C[C@H]1CCNC1] | —Ac | 1-[(3R)-3-{[(3R)-3- ({3-(7-fluoro-3- (propan-2-yl)-1H- indazol-1-yl]- 1,2,4-oxadiazol-5- yl}methyl)pyrrolidin- 1-yl]methyl}pyrrolidin- 1-yl]ethanone | ¹H-NMR (CDCl₃) δ: 1.49 (6H, d, J = 7.1 Hz), 1.52-1.78 (2H, m), 1.92-2.21 (5H, m), 2.29-2.83 (8H, m), 3.01-3.19 (3H, m), 3.30-3.73 (4H, m), 7.17-7.27 (2H, m), 7.55-7.61 (1H, m). LC-MS, m/z; 455 [M + H]+ |
| 506 | | —Ms | 7-fluoro-1-(5- {[(3R)-1-{[(3R)-1- (methylsulfonyl) pyrrolidin-3- yl]methyl}pyrrolidin- 3-yl]methyl}- 1,2,4-oxadiazol-3- yl)-3-(propan-2- yl)-1H-indazole | ¹H-NMR (CDCl₃) δ: 1.50 (6H, d, J = 7.1 Hz), 1.57-1.75 (3H, m), 2.03-2.20 (2H, m), 2.38-2.85 (10H, m), 3.03-3.11 (3H, m), 3.27-3.54 (4H, m), 7.19-7.29 (2H, m), 7.56-7.63 (1H, m). LC-MS, m/z; 491 [M + H]+ |

### Preparations of Examples 507 to 518

The compounds in the following table (i.e. Examples 507 to 518) were prepared in the same manner as in Example 168 except that the 3-ethyl-1-(5-{1-[2-(piperidin-4-yl)ethyl]piperidin-4-yl}-1,2,4-oxadiazol-3-yl)-1H-indazole bis(trifluoroacetate) and methyl chloroformate were replaced with the corresponding starting compound and acid chloride (defined as R—Cl) or acetic anhydride, respectively.

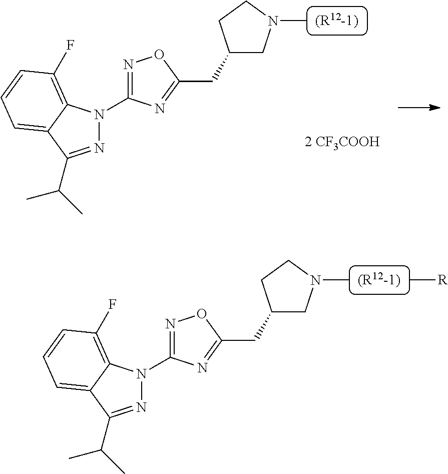

Wherein (R¹²-1) means each cyclic amino structure shown in the following table, and the structure of R is defined in the following table. R is attached to the nitrogen atom in the cyclic amine of (R¹²-1). Each free form of the compounds in the following table was obtained by omitting the conversion step into hydrochloride in Example 168.

TABLE 73

| Ex. | (R¹²-1) | R | Compound Name | ¹H-NMR/LC-MS, m/z |
|---|---|---|---|---|
| 507 | 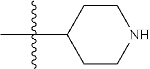 [SMILES: *C1CCNCC1] | —Ac | 1-{4-[(3S)-3-({3- [7-fluoro-3- (propan-2-yl)-1H- indazol-1-yl]- 1,2,4-oxadiazol-5- yl}methyl)pyrrolidin- 1-yl]piperidin- 1-yl}ethanone | ¹H-NMR (CDCl₃) δ: 1.39- 1.99 (12H, m), 2.08 (3H, s), 2.12-3.17 (10H, m), 3.41-3.55 (1H, m), 3.78 (1H, d, J = 13.6 Hz), 4.43 (1H, d, J = 10.5 Hz, 7.18- 7.30 (2H, m), 7.56-7.63 (1H, m). LC-MS, m/z; 455 [M + H]+ |
| 508 | | —Ms | 7-fluoro-1-[5- ({(3S)-1-[1- (methylsulfonyl) piperidin-4- yl]pyrrolidin-3- yl}methyl)-1,2,4- oxadiazol-3-yl]-3- (propan-2-yl)-1H- indazole | ¹H-NMR (CDCl₃) δ: 1.50 (6H, d, J = 7.0 Hz), 1.59-1.74 (3H, m), 1.87-1.99 (2H, m), 2.09-2.27 (2H, m), 2.42-2.51 (1H, m), 2.57-2.96 (9H, m), 3.08 (2H, d, J = 6.8 Hz), 3.42-3.54 (1H, m), 3.56-3.68 (2H, m), 7.18-7.30 (2H, m), 7.56-7.63 (1H, m). LC-MS, m/z; 491[M + H]+ |
| 509 | 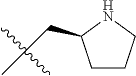 [SMILES: *C[C@@H]1CCCN1] | —Ac | 1-[(2S)-2-{[(3S)- 3-({3-[7-fluoro-3- (propan-2-yl)-1H- indazol-1-yl]- 1,2,4-oxadiazol-5- yl}methyl)pyrrolidin- 1-yl]methyl)pyrrolidin- 1-yl]ethanone | ¹H-NMR (CDCl₃) δ: 1.50 (6H, d, J = 7.0 Hz), 1.55-1.70 (1H, m), 1.77-2.20 (8H, m), 2.32-2.89 (7H, m), 2.98-3.14 (2H, m), 3.31-3.56 (3H, m), 3.80-3.95 (0.4H, m, rotamer), 4.13-4.26 (0.6H, m, rotamer), 7.18-7.29 (2H, m), 7.55-7.63 (1H, m). LC-MS, m/z; 455 [M + H]+ |
| 510 | | —Ms | 7-fluoro-1-(5- {[(3S)-1-{[(2S)-1- (methylsulfonyl) pyrrolidin-2- yl]methyl}pyrrolidin- 3-yl]methyl}- 1,2,4-oxadiazol-3- yl)-3-(propan-2- yl)-1H-indazole | ¹H-NMR (CDCl₃) δ: 1.50 (6H, d, J = 7.0 Hz), 1.54-1.69 (1H, m), 1.82-2.00 (4H, m), 2.05-2.21 (1H, m), 2.43-2.86 (7H, m), 2.88 (3H, s), 3.02-3.10 (2H, m), 3.29-3.41 (2H, m), 3.43-3.54 (1H, m), 3.75-3.87 (1H, m), 7.18-7.27 (2H, m), 7.56-7.63 (1H, m). LC-MS, m/z; 491 [M + H]+ |
| 511 | 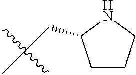 [SMILES: *C[C@H]1CCCN1] | —Ac | 1-[(2R)-2-{[(3S)- 3-({3-[7-fluoro-3- (propan-2-yl)-1H- indazol-1-yl]- 1,2,4-oxadiazol-5- yl}methyl)pyrrolidin- 1-yl]methyl}pyrrolidin- 1-yl]ethanone | ¹H-NMR (CDCl₃) δ: 1.50 (6H, d, J = 7.0 Hz), 1.55-1.71 (1H, m), 1.80-2.20 (8H, m), 2.32-2.88 (7H, m), 3.01-3.11 (2H, m), 3.31-3.57 (3H, m), 3.82-3.93 (0.4H, m, rotamer), 4.14-4.27 (0.6H, m, rotamer), 7.18-7.27 (2H, m), 7.55-7.63 (1H, m). LC-MS, m/z; 455 [M + H]+ |
| 512 | | —Ms | 7-fluoro-1-(5- {[(3S)-1-{[(2R)-1- (methylsulfonyl) pyrrolidin-2- yl]methyl}pyrrolidin- 3-yl]methyl}- 1,2,4-oxadiazol-3- yl)-3-(propan-2- yl)-1H-indazole | ¹H-NMR (CDCl₃) δ: 1.50 (6H, d, J = 7.1 Hz), 1.57-1.67 (1H, m), 1.84-2.00 (4H, m), 2.06-2.17 (1H, m), 2.46-2.68 (4H, m), 2.72-2.83 (3H, m), 2.88 (3H, s), 3.02-3.08 (2H, m), 3.29-3.41 (2H, m), 3.44-3.53 (1H, m), 3.75-3.85 (1H, m), 7.19-7.27 (2H, m), 7.56-7.62 (1H, m). LC-MS, m/z; 491 [M + H]+ |
| 513 | 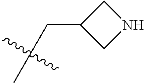 [SMILES: *CC1CNC1] | —Ac | 1-(3-{[(3S)-3-({3- [7-fluoro-3- (propan-2-yl)-1H- indazol-1-yl]- 1,2,4-oxadiazol-5- yl}methyl)pyrrolidin- 1-yl]methyl}azetidin- 1-yl)ethanone | ¹H-NMR (CDCl₃) δ: 1.50 (6H, d, J = 7.2 Hz), 1.56-1.72 (1H, m), 1.83 (3H, d, J = 1.7 Hz), 2.06-2.24 (1H, m), 2.37-2.85 (8H, m), 3.06 (2H, d, J = 6.4 Hz), 3.41-3.55 (1H, m), 3.59-3.68 (1H, m), 3.75-3.82 (1H, m), 4.02-4.23 (2H, m), 7.18-7.30 (2H, m), 7.55-7.63 (1H, m). LC-MS, m/z; 441 [M + H]+ |
| 514 | | —Ms | 7-fluoro-1-(5- {[(3S)-1-{[1- (methylsulfonyl) azetidin-3- yl]methyl}pyrrolidin- 3-yl]methyl}- 1,2,4-oxadiazol-3- yl)-3-(propan-2- yl)-1H-indazole | ¹H-NMR (CDCl₃) δ: 1.50 (6H, d, J = 7.0 Hz), 1.57-1.71 (1H, m), 2.07-2.22 (1H, m), 2.36-2.82 (8H, m), 2.84 (3H, s), 3.05 (2H, d, J = 7.2 Hz), 3.41-3.54 (1H, m), 3.66 (2H, dd, J = 7.8, 5.8 Hz), 3.93- 4.03 (2H, m), 7.19-7.29 (2H, m), 7.56-7.63 (1H, m). LC-MS, m/z; 477 [M + H]+ |
| 515 | 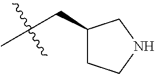 [SMILES: *C[C@@H]1CCNC1] | —Ac | 1-[(3S)-3-{[(3S- 3-({3-[7-fluoro-3- (propan-2-yl)-1H- indazol-1-yl]- 1,2,4-oxadiazol-5- yl}methyl)pyrrolidin- 1-yl]methyl}pyrrolidin- 1-yl]ethanone | ¹H-NMR (CDCl₃) δ: 1.49 (6H, d, J = 7.1 Hz), 1.52-1.78 (2H, m), 1.92-2.21 (5H, m), 2.29-2.83 (8H, m), 3.01-3.19 (3H, m), 3.30-3.73 (4H, m), 7.17-7.27 (2H, m), 7.55-7.61 (1H, m). LC-MS, m/z; 455 [M + H]+ |
| 516 | | —Ms | 7-fluoro-1-(5- {[(3S)-1-{[(3S)-1- (methylsulfonyl) pyrrolidin-3- yl]methyl}pyrrolidin- 3-yl]methyl}- 1,2,4-oxadiazol-3- yl)-3-(propan-2- yl)-1H-indazole | ¹H-NMR (CDCl₃) δ: 1.50 (6H, d, J = 7.1 Hz), 1.57-1.75 (3H, m), 2.03-2.20 (2H, m), 2.38-2.85 (10H, m), 3.03-3.11 (3H, m), 3.27-3.54 (4H, m), 7.19-7.29 (2H, m), 7.56-7.63 (1H, m). LC-MS, m/z; 491 [M + H]+ |
| 517 | 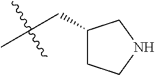 [SMILES: *C[C@H]1CCNC1] | —Ac | 1-[(3R)-3-{[(3S)- 3-({3-[7-fluoro-3- (propan-2-yl)-1H- indazol-1-yl]- 1,2,4-oxadiazol-5- yl}methyl)pyrrolidin- 1-yl]methyl}pyrrolidin- 1-yl]ethanone | ¹H-NMR (CDCl₃) δ: 1.44- 1.80 (8H, m), 1.95-2.23 (5H, m), 2.29-2.86 (8H, m), 3.02-3.21 (3H, m), 3.31-3.74 (4H, m), 7.18-7.28 (2H, m), 7.56-7.62 (1H, m). LC-MS, m/z; 455 [M + H]+ |
| 518 | 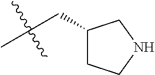 [SMILES: *C[C@H]1CCNC1] | —Ms | 7-fluoro-1-(5- {[(3S)-1-{[(3R)-1- (methylsulfonyl) pyrrolidin-3- yl]methyl}pyrrolidin- 3-yl]methyl}- 1,2,4-oxadiazol-3- yl)-3-(propan-2- yl)-1H-indazole | ¹H-NMR (CDCl₃) δ: 1.50 (6H, d, J = 7.0 Hz), 1.55-1.77 (2H, m), 1.99-2.24 (2H, m), 2.36-2.80 (8H, m), 2.82 (3H, s), 3.01-3.13 (3H, m), 3.24-3.56 (4H, m), 7.18-7.30 (2H, m), 7.56-7.63 (1H, m). LC-MS, m/z; 491 [M + H]+ |

### Preparations of Examples 519 to 528

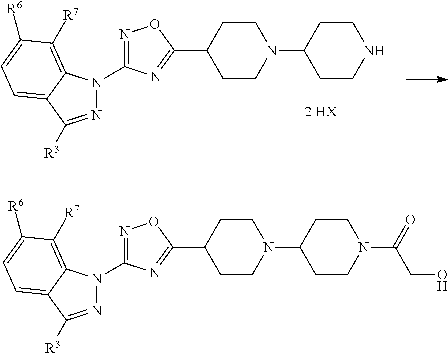

Wherein HX is hydrochloric acid or trifluoroacetic acid.

The compounds in the following table (i.e. Examples 519 to 528) were prepared in the same manner as in Example 242 or the 2 N sodium hydroxide aqueous solution was replaced with methylamine/methanol solution, provided that the 4-{3-[7-fluoro-3-(propan-2-yl)-1H-indazol-1-yl]-1,2,4-oxadiazol-5-yl}-1,4′-bipiperidine dihydrochloride of Example 242 was replaced with the corresponding starting compound. Each of the hydrochlorides in the following table was obtained by dissolving the resultant compound in methylene chloride and then treating with 1N HCl/diethyl ether solution. In order to obtain each of the trifluoroacetates, the residue was isolated/purified by reverse phase HPLC.

TABLE 74

| Ex. | R³ | R⁶ | R⁷ | Compound Name | ¹H-NMR/LC-MS, m/z |
|---|---|---|---|---|---|
| 519 | ⁱPr | H | H | 2-hydroxy-1-(4-{3- 3-(propan-2-yl)- 1H-indazol-1-yl]- 1,2,4-oxadiazol-5- yl}-1,4′- bipiperidin-1′- yl)ethanone | LC-MS, m/z; 453 [M + H]+ |
| 520 | ⁱPr | F | F | 1-(4-{3-[6,7- difluoro-3- (propan-2-yl)-1H- indazol-1-yl]- 1,2,4-oxadiazol-5- yl}-1,4′- bipiperidin-1′- yl)-2- hydroxyethanone | ¹H-NMR (CDCl₃) δ: 1.35- 1.55 (6H, m), 1.55-1.87 (2H, m), 2.00-2.80 (7H, m), 2.90-3.15 (2H, m), 3.15-3.90 (7H, m), 4.00- 4.37 (2H, m), 4.60-4.85 (1H, m), 6.90-7.30 (1H, m), 7.42-7.60 (1H, m). A signal due to OH was not observed. LC-MS, m/z; 489 [M + H]+ |
| 521 | 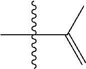 [SMILES: *C(=C)C] | H | F | 1-(4-{3-[7-fluoro- 3-(prop-1-en-2- yl)-1H-indazol-1- yl]-1,2,4- oxadiazol-5-yl}- 1,4′-bipiperidin- 1'-yl)-2- hydroxyethanone hydrochloride | ¹H-NMR (DMSO-d₆) δ: 1.46- 1.80 (2H, m), 2.08-2.70 (9H, m), 2.89-3.67 (8H, m), 3.81-3.95 (1H, m), 4.02-4.21 (2H, m), 4.41- 4.77 (2H, m), 5.67 (1H, s), 6.00 (1H, s), 7.39- 7.59 (2H, m), 7.95-8.04 (1H, m), 11.09-11.28 (1H, bs). LC-MS, m/z; 469 [M + H]+ |
| 522 | 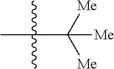 [SMILES: *C(C)(C)C] | H | H | 1-{4-[3-(3-tert- butyl-1H-indazol- 1-yl)-1,2,4- oxadiazol-5-yl]- 1,4′-bipiperidin- 1′-yl}-2- hydroxyethanone | ¹H-NMR (CDCl₃) δ: 1.42- 1.67 (11H, m), 1.82-2.26 (7H, m), 2.33-2.46 (2H, m), 2.53-2.82 (2H, m), 2.91-3.14 (4H, m), 3.57 (1H, d, J = 13.8 Hz), 4.17 (2H, s), 4.63 (1H, d, J = 13.8 Hz), 7.24- 7.35 (1H, m), 7.53 (1H, t, J = 7.8 Hz), 7.94 (1H, d, J = 8.3 Hz), 8.32 (1H, d, J = 8.4 Hz). LC-MS, m/z; 467 [M + H]+ |
| 523 | 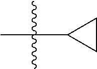 [SMILES: *C1CC1] | H | F | 1-{4-[3-(3- cyclopropyl-7- fluoro-1H-indazol- 1-yl)-1,2,4- oxadiazol-5-yl]- 1,4′-bipiperidin- 1′-yl}-2- hydroxyethanone | ¹H-NMR (CDCl₃) δ: 1.07- 1.18 (2H, m), 1.19-1.27 (2H, m), 1.40-1.58 (2H, m), 1.83-2.11 (4H, m), 2.13-2.47 (5H, m), 2.52- 2.65 (1H, m), 2.67-2.80 (1H, m), 2.91-3.10 (4H, m), 3.50-3.75 (2H, m), 4.16 (2H, s), 4.55-4.69 (1H, m), 7.18-7.29 (2H, m), 7.55-7.62 (1H, m). LC-MS, m/z; 469 [M + H]+ |
| 524 | ⁿPr | H | F | 1-{4-[3-(7-fluoro- 3-propyl-1H- indazol-1-yl)- 1,2,4-oxadiazol-5- yl]-1,4′- bipiperidin-1′- yl}-2- hydroxyethanone | ¹H-NMR (CDCl₃) δ: 1.04 (3H, t, J = 7.3 Hz), 1.41-1.59 (2H, m), 1.81- 1.95 (4H, m), 1.96-2.12 (2H, m), 2.14-2.26 (2H, m), 2.32-2.47 (2H, m), 2.53-2.65 (1H, m), 2.67- 2.80 (1H, m), 2.92-3.11 (6H, m), 3.50-3.76 (2H, m), 4.16 (2H, s), 4.56- 4.68 (1H, m), 7.19-7.30 (2H, m), 7.50-7.57 (1H, m). LC-MS, m/z; 471 [M + H]+ |
| 525 | Me | H | F | 1-{4-[3-(7-fluoro- 3-methyl-1H- indazol-1-yl)- 1,2,4-oxadiazol-5- yl]-1,4′- bipiperidin-1′- yl}-2- hydroxyethanone | ¹H-NMR (CDCl₃) δ: 1.40- 1.59 (2H, m), 1.84-2.12 (4H, m), 2.15-2.26 (2H, m), 2.34-2.47 (2H, m), 2.53-2.80 (5H, m), 2.92- 3.11 (4H, m), 3.50-3.74 (2H, m), 4.16 (2H, s), 4.56-4.68 (1H, m), 7.21- 7.31 (2H, m), 7.48-7.53 (1H, m). LC-MS, m/z; 443 [M + H]+ |
| 526 | 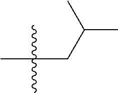 [SMILES: *CC(C)C] | H | F | 1-(4-{3-[7-fluoro- 3-(2- methylpropyl)-1H- indazol-1-yl]- 1,2,4-oxadiazol-5- yl}-1,4′- bipiperidin-1′- yl)-2- hydroxyethanone | ¹H-NMR (CDCl₃) δ: 1.00 (6H, d, J = 6.8 Hz), 1.41-1.59 (2H, m), 1.82- 2.11 (4H, m), 2.14-2.32 (3H, m), 2.33-2.47 (2H, m), 2.53-2.81 (2H, m), 2.87-3.12 (6H, m), 3.50- 3.76 (2H, m), 4.16 (2H, s), 4.57-4.68 (1H, m), 7.19-7.29 (2H, m), 7.49- 7.55 (1H, m). LC-MS, m/z; 485 [M + H]+ |
| 527 | 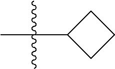 [SMILES: *C1CCC1] | H | F | 1-{4-[3-(3- cyclobutyl-7- fluoro-1H-indazol- 1-yl)-1,2,4- oxadiazol-5-yl]- 1,4′-bipiperidin- 1′-yl}-2- hydroxyethanone | ¹H-NMR (CDCl₃) δ: 1.41- 1.60 (2H, m), 1.85-2.29 (8H, m), 2.34-2.81 (8H, m), 2.91-3.14 (4H, m), 3.43-3.82 (2H, m), 3.91- 4.05 (1H, m), 4.16 (2H, s), 4.57-4.69 (1H, m), 7.17-7.29 (2H, m), 7.49- 7.56 (1H, m). LC-MS, m/z; 483 [M + H]+ |
| 528 | 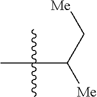 [SMILES: *C(C)CC] | H | F | 1-(4-{3-[3-(butan- 2-yl)-7-fluoro-1H- indazol-1-yl]- 1,2,4-oxadiazol-5- yl}-1,4′- bipiperidin-1′- yl)-2- hydroxyethanone | ¹H-NMR (CDCl₃) δ: 0.93 (3H, t, J = 7.4 Hz), 1.42-1.57 (4H, m), 1.76- 2.10 (6H, m), 2.15-2.24 (2H, m), 2.33-2.44 (2H, m), 2.54-2.64 (1H, m), 2.69-2.82 (2H, m), 2.93- 3.10 (4H, m), 3.19-3.30 (1H, m), 3.51-3.71 (2H, m), 4.16 (2H, s), 4.58- 4.67 (1H, m), 7.18-7.28 (2H, m), 7.55-7.60 (1H, m). LC-MS, m/z; 485 [M + H]+ |

### Preparations of Examples 529 to 538

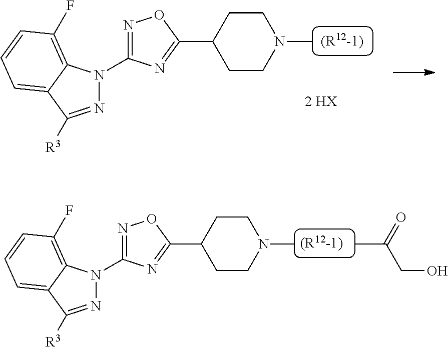

Wherein HX is hydrochloric acid or trifluoroacetic acid, and (R¹²-1) means each cyclic amino structure shown in the following table. The hydroxyacetyl group is attached to the nitrogen atom in the cyclic amine of (R¹²-1).

The compounds in the following table (i.e. Examples 529 to 538) were prepared in the same manner as in Example 242 or the 2 N sodium hydroxide aqueous solution was replaced with methylamine/methanol, provided that the 4-{3-[7-fluoro-3-(propan-2-yl)-1H-indazol-1-yl]-1,2,4-oxadiazol-5-yl}-1,4′-bipiperidine dihydrochloride was replaced with the corresponding starting compound. Each of the hydrochlorides in the following table was obtained by dissolving the resultant compound in methylene chloride and then treating with 1N HCl/diethyl ether solution.

TABLE 75

| Ex. | R³ | (R¹²-1) | Compound Name | ¹H-NMR/LC-MS, m/z |
|---|---|---|---|---|
| 529 | ⁱPr | 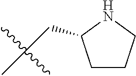 [SMILES: *C[C@H]1CCCN1] | 1-{(2R)-2-[(4-{3-[7- fluoro-3-(propan-2- yl)-1H-indazol-1- yl]-1,2,4-oxadiazol- 5-yl}piperidin-1- yl)methyl]pyrrolidin- 1-yl}-2- hydroxyethanone hydrochloride | ¹H-NMR (DMSO-d₆) δ: 1.41 (6H, d, J = 7.0 Hz), 1.80-2.48 (7H, m), 3.04-4.12 (14H, m), 4.36- 4.58 (1H, m), 7.34-7.55 (2H, m), 7.84 (1H, d, J = 7.7 Hz), 10.14- 10.51 (1H, br m). LC-MS, m/z; 471 [M + H]+ |
| 530 | ⁱPr | 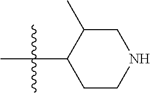 [SMILES: *C1CCNCC1C] | 1-(4-{3-[7-fluoro-3- (propan-2-yl)-1H- indazol-1-yl]-1,2,4- oxadiazol-5-yl}-3′- methyl-1,4′- bipiperidin-1′-yl)- 2-hydroxyethanone | ¹H-NMR (CDCl₃) δ: 0.91 (3H, t, J = 7.4 Hz), 1.44-1.66 (2H, m), 1.51 (6H, d, J = 7.1 Hz), 1.79-1.89 (1H, m), 1.97-2.33 (7H, m), 2.55-3.16 (5H, m), 3.27-3.60 (2H, m), 3.61-3.70 (1H, m), 4.06-4.29 (2H, m), 4.48-4.73 (1H, m), 7.18-7.29 (2H, m), 7.55-7.63 (1H, m). LC-MS, m/z; 485 [M + H]+ |
| 531 | ⁱPr | 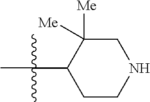 [SMILES: *C1CCNCC1(C)C] | 1-(4-{3-[7-fluoro-3- (propan-2-yl)-1H- indazol-1-yl]-1,2,4- oxadiazol-5-yl}-3′, 3′-dimethyl-1,4′- bipiperidin-1′-yl)- 2-hydroxyethanone | ¹H-NMR (CDCl₃) δ: 0.85-0.92 (3H, m), 0.97-1.06 (3H, m), 1.50 (6H, t, J = 3.6 Hz), 1.68-1.80 (2H, m), 1.90-2.43 (6H, m), 2.52-3.71 (9H, m), 4.04-4.80 (3H, m), 7.16-7.30 (2H, m), 7.55-7.62 (1H, m). LC-MS, m/z; 499 [M + H]+ |
| 532 and 533 | ⁱPr | 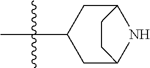 [SMILES: *C1CC2CCC(C1)N2] | 1-[(3-endo)-3-(4-{3- [7-fluoro-3-(propan- 2-yl)-1H-indazol-1- yl]-1,2,4-oxadiazol- 5-yl}piperidin-1- yl)-8- azabicyclo[3.2.1]oct- 8-yl]-2- hydroxyethanone and | ¹H-NMR (CDCl₃) δ: 1.50 (6H, d, J = 7.1 Hz), 1.81-2.27 (14H, m), 2.33- 2.41 (1H, m), 2.98-3.25 (3H, m), 3.42-3.58 (2H, m), 3.88-3.95 (1H, m), 4.11 (2H, dd, J = 28.7, 14.8 Hz), |
| | | | 1-[(3-exo)-3-(4-{3- [7-fluoro-3-(propan- 2-yl)-1H-indazol-1- yl]-1,2,4-oxadiazol- 5-yl}piperidin-1- yl)-8- azabicyclo[3.2.1]oct- 8-yl]-2- hydroxyethanone | 4.62-4.70 (1H, m), 7.18-7.29 (2H, m), 7.56-7.62 (1H, m). LC-MS, m/z; 497 [M + H]+ and ¹H-NMR (CDCl₃) δ: 1.50 (6H, d, J = 6.8 Hz), 1.53-1.67 |
| | | | | (2H, m), 1.67-2.08 (8H, m), 2.13-2.23 (2H, m), 2.26-2.37 (2H, m), 2.83-3.09 (4H, m), 3.42-3.58 (2H, m), 3.99-4.21 (3H, m), 4.75-4.82 (1H, m), 7.18-7.30 (2H, m), 7.56-7.62 (1H, m). LC-MS, m/z; 497 [M + H]+ |
| 534 | ⁱPr | 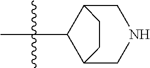 [SMILES: *C1C2CCC1CNC2] | 1-[(8-anti)-8-(4-(3- [7-fluoro-3-(propan- 2-yl)-1H-indazol-1- yl]-1,2,4-oxadiazol- 5-yl}piperidin-1- yl)-3- azabicyclo[3.2.1]oct- 3-yl]-2- hydroxyethanone | ¹H-NMR (CDCl₃) δ: 1.42-1.59 (7H, m), 1.73-1.88 (2H, m), 1.99-2.37 (10H, m), 2.90 (1H, d, J = 11.0 Hz), 2.97- 3.16 (3H, m), 3.26 (1H, d, J = 12.3 Hz), 3.41-3.59 (2H, m), 3.78 (1H, t, J = 4.3 Hz), 3.93-4.10 (2H, m), 4.18-4.29 (1H, m), 7.17-7.30 (2H, m), 7.55-7.63 (1H, m). LC-MS, m/z; 497 [M + H]+ |
| 535 and 536 | ⁱPr | 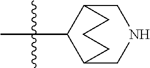 [SMILES: *C1C2CCCC1CNC2] | 1-[(9-anti)-9-(4-{3- [7-fluoro-3-(propan- 2-yl)-1H-indazol-1- yl]-1,2,4-oxadiazol- 5-yl}piperidin-1- yl)-3- azabicyclo[3.3.1]non- 3-yl]-2- hydroxyethanone and 1-[(9-syn)-9-(4-{3- [7-fluoro-3-(propan- 2-yl)-1H-indazol-1- yl]-1,2,4-oxadiazol- 5-yl}piperidin-1- yl)-3- azabicyclo[3.3.1]non- 3-yl]-2- hydroxyethanone | ¹H-NMR (CDCl₃) δ: 1.38-1.61 (9H, m), 1.89-2.28 (12H, m), 2.94-3.31 (5H, m), 3.42-3.62 (2H, m), 3.69-3.80 (1H, m), 4.08-4.29 (2H, m), 4.60-4.69 (1H, m), 7.18-7.30 (2H, m), 7.57-7.63 (1H, m). LC-MS, m/z; 511 [M + H]+ And LC-MS, m/z; 511 [M + H]+ |
| 537 | ⁱPr | 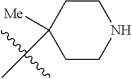 [SMILES: *C1(C)CCNCC1] | 1-(4-{3-[7-fluoro-3- (propan-2-yl)-1H- indazol-1-yl]-1,2,4- oxadiazol-5-yl}-4′- methyl-1,4′- bipiperidin-1′-yl)- 2-hydroxyethanone | ¹H-NMR (CDCl₃) δ: 0.96 (3H, s), 1.32-1.44 (2H, m), 1.47-1.58 (6H, m), 1.89-2.09 (4H, m), 2.13-2.37 (4H, m), 2.93-3.19 (4H, m), 3.22-3.55 (3H, m), 3.73 (1H, t, J = 4.2 Hz), 4.00-4.23 (3H, m), 7.17-7.30 (2H, m), 7.55-7.63 (1H, m). LC-MS, m/z; 485 [M + H]+ |
| 538 | Et | 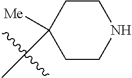 [SMILES: *C1(C)CCNCC1] | 1-{4-[3-(3-ethyl-7- fluoro-1H-indazol-1- yl)-1,2,4-oxadiazol- 5-yl]-4′-methyl- 1,4′-bipiperidin-1′- yl}-2- hydroxyethanone | ¹H-NMR (CDCl₃) δ: 0.96 (3H, s), 1.30-1.51 (5H, m), 1.87-2.08 (4H, m), 2.11-2.37 (4H, m), 2.88-3.15 (6H, m), 3.19-3.41 (2H, m), 3.71 (1H, t, J = 4.2 Hz), 3.98-4.08 (1H, m), 4.10-4.21 (2H, m), 7.18-7.29 (2H, m), 7.48-7.54 (1H, m). LC-MS, m/z; 471 [M + H]+ |

### Preparations of Examples 539 to 544

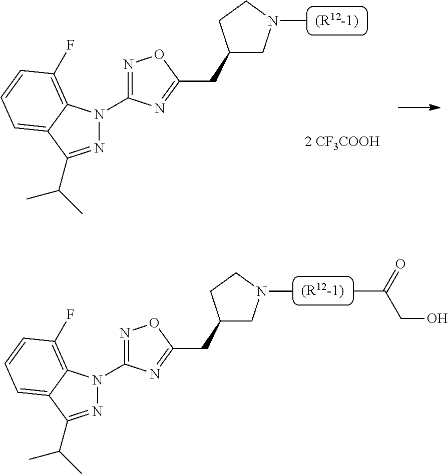

Wherein (R¹²-1) means each cyclic amino structure shown in the following table. The hydroxyacetyl group is attached to the nitrogen atom in the cyclic amine of (R¹²-1).

The compounds in the following table (i.e. Examples 539 to 544) were prepared in the same manner as in Example 242 or the 2 N sodium hydroxide aqueous solution was replaced with methylamine/methanol, provided that the 4-{3-[7-fluoro-3-(propan-2-yl)-1H-indazol-1-yl]-1,2,4-oxadiazol-5-yl}-1,4′-bipiperidine dihydrochloride was replaced with the corresponding starting compound.

TABLE 76

| Ex. | (R¹²-1) | Compound Name | ¹H-NMR/LC-MS, m/z |
|---|---|---|---|
| 539 | 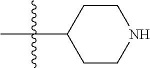 [SMILES: *C1CCNCC1] | 1-{4-[(3R)-3-({3-[7- fluoro-3-(propan-2- yl)-1H-indazol-1-yl]- 1,2,4-oxadiazol-5- yl}methyl)pyrrolidin- 1-yl]piperidin-1-yl}- 2-hydroxyethanone | ¹H-NMR (CDCl₃) δ: 1.40- 1.58 (8H, m), 1.59-1.74 (1H, m), 1.81-1.95 (2H, m), 2.08-3.15 (11H, m), 3.40-3.56 (3H, m), 4.14 (2H, s), 4.20-4.38 (1H, m), 7.18-7.30 (2H, m), 7.55-7.64 (1H, m). LC-MS, m/z; 471 [M + H]+ |
| 540 | 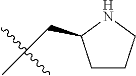 [SMILES: *C[C@@H]1CCCN1] | 1-[(2S)-2-{[(3R)-3- ({3-[7-fluoro-3- (propan-2-yl)-1H- indazol-1-yl]-1,2,4- oxadiazol-5- yl}methyl)pyrrolidin- 1- yl]methyl}pyrrolidin- 1-yl]-2- hydroxyethanone | ¹H-NMR (CDCl₃) δ: 1.50 (6H, d, J = 7.2 Hz), 1.59-1.79 (1H, m), 1.84- 2.26 (5H, m), 2.46-3.56 (14H, m), 3.99-4.35 (2H, m), 7.18-7.30 (2H, m), 7.55-7.63 (1H, m). LC-MS, m/z; 471 [M + H]+ |
| 541 | 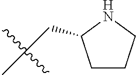 [SMILES: *C[C@H]1CCCN1] | 1-[(2R)-2-{[(3R)-3- ({3-[7-fluoro-3- (propan-2-yl)-1H- indazol-1-yl]-1,2,4- oxadiazol-5- yl}methyl)pyrrolidin- 1- yl]methyl}pyrrolidin- 1-yl]-2- hydroxyethanone | ¹H-NMR (CDCl₃) δ: 1.50 (6H, d, J = 7.0 Hz), 1.56-1.73 (1H, m), 1.81- 2.24 (5H, m), 2.32-3.38 (12H, m), 3.41-3.56 (2H, m), 3.99-4.35 (2H, m), 7.18-7.30 (2H, m), 7.55- 7.63 (1H, m). LC-MS, m/z; 471 [M + H]+ |
| 542 | 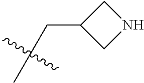 [SMILES: *CC1CNC1] | 1-(3-{[(3R)-3-({3-[7- fluoro-3-(propan-2- yl)-1H-indazol-1-yl]- 1,2,4-oxadiazol-5- yl}methyl)pyrrolidin- 1-yl]methyl}azetidin- 1-yl)-2- hydroxyethanone | ¹H-NMR (CDCl₃) δ: 1.50 (6H, d, J = 7.1 Hz), 1.57-1.75 (2H, m), 2.08- 2.21 (1H, m), 2.38-2.92 (8H, m), 3.06 (2H, d, J = 6.8 Hz), 3.41-3.55 (1H, m), 3.67-3.79 (2H, m), 3.94 (2H, br s), 4.05- 4.22 (2H, m), 7.20-7.29 (2H, m), 7.57-7.63 (1H, m). LC-MS, m/z; 457 [M + H]+ |
| 543 | 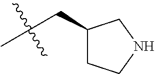 [SMILES: *C[C@@H]1CCNC1] | 1-[(3S)-3-{[(3R)-3- ({3-[7-fluoro-3- (propan-2-yl)-1H- indazol-1-yl]-1,2,4- oxadiazol-5- yl}methyl)pyrrolidin- 1- yl]methyl}pyrrolidin- 1-yl]-2- hydroxyethanone | ¹H-NMR (CDCl₃) δ: 1.50 (6H, d, J = 7.1 Hz), 1.53-1.82 (2H, m), 1.99- 2.21 (2H, m), 2.32-2.85 (9H, m), 3.07 (2H, d, J = 7.1 Hz), 3.16-3.54 (4H, m), 3.63-3.80 (1H, m), 4.07 (2H, br s), 7.18- 7.29 (2H, m), 7.56-7.63 (1H, m). LC-MS, m/z; 471 [M + H]+ |
| 544 | 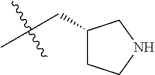 [SMILES: *C[C@H]1CCNC1] | 1-[(3R)-3-{[(3R)-3- ({3-[7-fluoro-3- (propan-2-yl)-1H- indazol-1-yl]-1,2,4- oxadiazol-5- yl}methyl)pyrrolidin- 1- yl]methyl}pyrrolidin- 1-yl]-2- hydroxyethanone | ¹H-NMR (CDCl₃) δ: 1.50 (6H, d, J = 7.1 Hz), 1.54-1.82 (2H, m), 1.99- 2.22 (2H, m), 2.33-2.85 (9H, m), 3.00-3.11 (2H, m), 3.17-3.53 (4H, m), 3.62-3.81 (1H, m), 4.01- 4.13 (2H, m), 7.19-7.29 (2H, m), 7.56-7.62 (1H, m). LC-MS, m/z; 471 [M + H]+ |

### Preparations of Examples 545 to 550

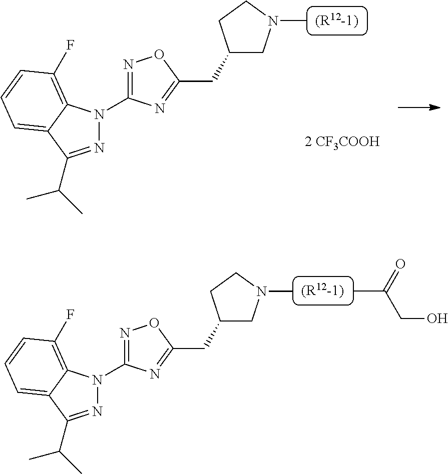

Wherein (R¹²-1) means each cyclic amino structure shown in the following table. The hydroxyacetyl group is attached to the nitrogen atom in the cyclic amine of (R¹²-1).

The compounds in the following table (i.e. Examples 545 to 550) were prepared in the same manner as in Example 242 or the 2 N sodium hydroxide aqueous solution was replaced with methylamine/methanol, provided that the 4-[(3-[7-fluoro-3-(propan-2-yl)-1H-indazol-1-yl]-1,2,4-oxadiazol-5-yl]-1,4′-bipiperidine dihydrochloride was replaced with the corresponding starting compound.

TABLE 77

| Ex. | (R¹²-1) | Compound Name | ¹H-NMR/LC-MS, m/z |
|---|---|---|---|
| 545 | 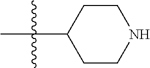 [SMILES: *C1CCNCC1] | 1-{4-[(3S)-3-({3-[7- fluoro-3-(propan-2- yl)-1H-indazol-1- yl]-1,2,4-oxadiazol- 5- yl}methyl)pyrrolidin- 1-yl]piperidin-1- yl}-2- hydroxyethanone | ¹H-NMR (CDCl₃) δ: 1.40- 1.58 (8H, m), 1.59-1.74 (1H, m), 1.81-1.95 (2H, m), 2.08-3.15 (11H, m), 3.40-3.56 (3H, m), 4.14 (2H, s), 4.20-4.38 (1H, m), 7.18-7.30 (2H, m), 7.55-7.64 (1H, m). LC-MS, m/z; 471 [M + H]+ |
| 546 | 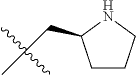 [SMILES: *C[C@@H]1CCCN1] | 1-[(2S)-2-{[(3S)-3- ({3-[7-fluoro-3- (propan-2-yl)-1H- indazol-1-yl]-1,2,4- oxadiazol-5- yl}methyl)pyrrolidin- 1- yl]methyl}pyrrolidin- 1-yl]-2- hydroxyethanone | ¹H-NMR (CDCl₃) δ: 1.50 (6H, d, J = 7.0 Hz), 1.56-1.73 (1H, m), 1.81- 2.24 (5H, m), 2.32-3.38 (12H, m), 3.41-3.56 (2H, m), 3.99-4.35 (2H, m), 7.18-7.30 (2H, m), 7.55- 7.63 (1H, m). LC-MS, m/z; 471 [M + H]+ |
| 547 | 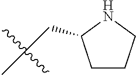 [SMILES: *C[C@H]1CCCN1] | 1-[(2R)-2-{[(3S)-3- ({3-[7-fluoro-3- (propan-2-yl)-1H- indazol-1-yl]-1,2,4- oxadiazol-5- yl}methyl)pyrrolidin- 1- yl]methyl}pyrrolidin- 1-yl]-2- hydroxyethanone | ¹H-NMR (CDCl₃) δ: 1.50 (6H, d, J = 7.2 Hz), 1.59-1.79 (1H, m), 1.84- 2.26 (5H, m), 2.46-3.56 (14H, m), 3.99-4.35 (2H, m), 7.18-7.30 (2H, m), 7.55-7.63 (1H, m). LC-MS, m/z; 471 [M + H]+ |
| 548 | 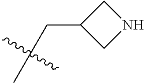 [SMILES: *CC1CNC1] | 1-(3-{[(3S)-3-({3- [7-fluoro-3-(propan- 2-yl)-1H-indazol-1- yl]-1,2,4-oxadiazol- 5- yl}methyl)pyrrolidin- 1- yl]methyl}azetidin- 1-yl)-2- hydroxyethanone | ¹H-NMR (CDCl₃) δ: 1.50 (6H, d, J = 7.1 Hz), 1.57-1.75 (2H, m), 2.08- 2.21 (1H, m), 2.38-2.92 (8H, m), 3.06 (2H, d, J = 6.8 Hz), 3.41-3.55 (1H, m), 3.67-3.79 (2H, m), 3.94 (2H, br s), 4.05- 4.22 (2H, m), 7.20-7.29 (2H, m), 7.57-7.63 (1H, m). LC-MS, m/z; 457 [M + H]+ |
| 549 | 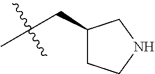 [SMILES: *C[C@@H]1CCNC1] | 1-[(3S)-3-{[(3S)-3- ({3-[7-fluoro-3- (propan-2-yl)-1H- indazol-1-yl]-1,2,4- oxadiazol-5- yl}methyl)pyrrolidin- 1- yl]methyl}pyrrolidin- 1-yl]-2- hydroxyethanone | ¹H-NMR (CDCl₃) δ: 1.50 (6H, d, J = 7.1 Hz), 1.54-1.82 (2H, m), 1.99- 2.22 (2H, m), 2.33-2.85 (9H, m), 3.00-3.11 (2H, m), 3.17-3.53 (4H, m), 3.62-3.81 (1H, m), 4.01- 4.13 (2H, m), 7.19-7.29 (2H, m), 7.56-7.62 (1H, m). LC-MS, m/z; 471 [M + H]+ |
| 550 | 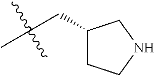 [SMILES: *C[C@H]1CCNC1] | 1-[(3R)-3-{[(3S)-3- ({3-[7-fluoro-3- (propan-2-yl)-1H- indazol-1-yl]-1,2,4- oxadiazol-5- yl}methyl)pyrrolidin- 1- yl]methyl}pyrrolidin- 1-yl]-2- hydroxyethanone | ¹H-NMR (CDCl₃) δ: 1.50 (6H, d, J = 7.1 Hz), 1.53-1.82 (2H, m), 1.99- 2.21 (2H, m), 2.32-2.85 (9H, m), 3.07 (2H, d, J = 7.1 Hz), 3.16-3.54 (4H, m), 3.63-3.80 (1H, m), 4.07 (2H, br s), 7.18- 7.29 (2H, m), 7.56-7.63 (1H, m). LC-MS, m/z; 471 [M + H]+ |

### Example 551

### Preparation of 1-(4-{3-[7-fluoro-3-(propan-2-yl)-1H-indazol-1-yl]-1,2,4-oxadiazol-5-yl}-4-hydroxy-1,4′-bipiperidin-1′-yl)-2-hydroxyethanone

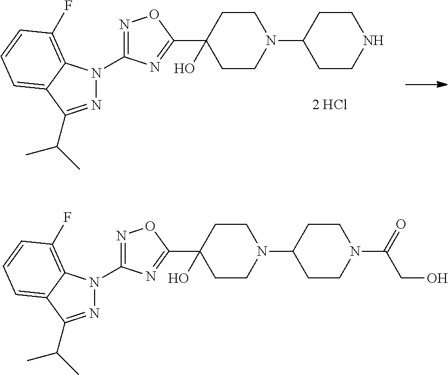 [SMILES: CC(C)c1nn(-c2noc(C3(O)CCN(C4CCNCC4)CC3)n2)c2c(F)cccc12 ; "2 HCl" ; CC(C)c1nn(-c2noc(C3(O)CCN(C4CCN(C(=O)CO)CC4)CC3)n2)c2c(F)cccc12]

The title compound was prepared in the same manner as in Example 242 except that the 4-{3-[7-fluoro-3-(propan-2-yl)-1H-indazol-1-yl]-1,2,4-oxadiazol-5-yl}-1,4′-bipiperidine dihydrochloride was replaced with 4-{3-[fluoro-3-(propan-2-yl)-1H-indazol-1-yl]-1,2,4-oxadiazol-5-yl}-1,4′-bipiperidin-4-ol dihydrochloride and the 2 N sodium hydroxide aqueous solution was replaced with methylamine/methanol.

LC-MS, m/z; 487 [M+H]+

### Example 552

### Preparation of 1-(4-{3-[7-fluoro-6-hydroxy-3-(propan-2-yl)-1H-indazol-1-yl]-1,2,4-oxadiazol-5-yl}-1,4′-bipiperidin-1′-yl)-2-hydroxyethanone

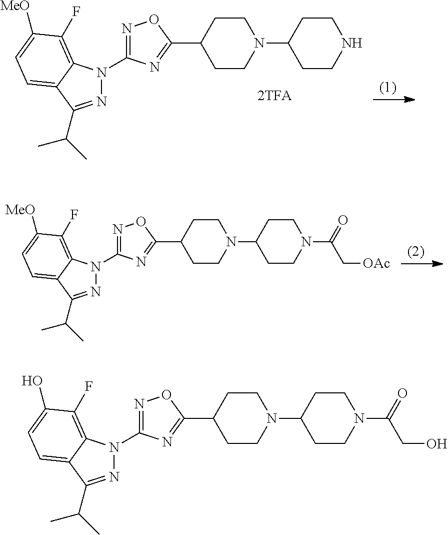 [SMILES: COc1ccc2c(C(C)C)nn(-c3noc(C4CCN(C5CCNCC5)CC4)n3)c2c1F ; COc1ccc2c(C(C)C)nn(-c3noc(C4CCN(C5CCN(C(=O)COC(C)=O)CC5)CC4)n3)c2c1F ; CC(C)c1nn(-c2noc(C3CCN(C4CCN(C(=O)CO)CC4)CC3)n2)c2c(F)c(O)ccc12]

(1) To a mixture of 4-{3-[7-fluoro-6-methoxy-3-(propan-2-yl)-1H-indazol-1-yl]-1,2,4-oxadiazol-5-yl}-1,4′-bipiperidine bis(trifluoroacetate) (250 mg), dichloromethane (5.0 ml) and saturated sodium bicarbonate aqueous solution (5.0 ml) was added dropwise acetoxyacetyl chloride (60 μl) at ice temperature, and the mixture was stirred for 30 minutes. To the solution was added saturated sodium bicarbonate, and the mixture was extracted with ethyl acetate. The organic layer was washed with water, dried, evaporated under reduced pressure, and the residue was purified by silica-gel chromatography (column; Hi-Flash™, developing solvent: chloroform/methanol=20:1) to give 2-(4-{3-[7-fluoro-6-methoxy-3-(propan-2-yl)-1H-indazol-1-yl]-1,2,4-oxadiazol-5-yl}-1,4′-bipiperidin-1′-yl)-2-oxoethyl acetate (207 mg).

(2) The 2-(4-{3-[7-fluoro-6-methoxy-3-(propan-2-yl)-1H-indazol-1-yl]-1,2,4-oxadiazol-5-yl}-1,4′-bipiperidin-1′-yl)-2-oxoethyl acetate (162 mg) was dissolved in dichloromethane (15 ml). To the solution was added 1N BBr₃ (in dichloromethane, 3.59 ml), and the mixed solution was stirred at room temperature overnight and then cooled at ice temperature. To the reaction solution was added saturated sodium bicarbonate aqueous solution, and the mixture was extracted with chloroform. The organic layer was dried, concentrated, and the residue was purified by silica-gel chromatography (column; Hi-Flash™, developing solvent: chloroform/methanol=10:1) to give the title compound (112 mg).

¹H-NMR (CDCl₃) δ: 1.39-1.59 (9H, m), 1.83-2.25 (6H, m), 2.32-2.48 (2H, m), 2.55-2.78 (2H, m), 2.91-3.08 (4H, m), 3.35-3.44 (1H, m), 3.56 (1H, d, J=13.8 Hz), 4.16 (2H, s), 4.63 (1H, d, J=13.0 Hz), 5.30 (1H, s), 6.99 (1H, dd, J=8.6, 6.8 Hz), 7.41 (1H, dd, J=8.6, 0.7 Hz).

LC-MS, m/z; 487 [M+H]+

### Example 553

### Preparation of 1-(4-{3-[7-fluoro-4-hydroxy-3-(propan-2-yl)-1,1′-indazol-1-yl]-1,2,4-oxadiazol-5-yl}-1,4′-bipiperidin-1′-yl)-2-hydroxyethanone

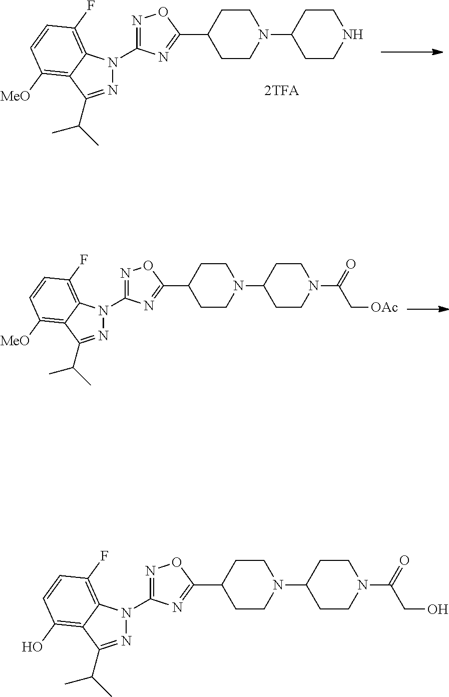 [SMILES: COc1ccc(F)c2c1c(C(C)C)nn2-c1noc(C2CCN(C3CCNCC3)CC2)n1 ; COc1ccc(F)c2c1c(C(C)C)nn2-c1noc(C2CCN(C3CCN(C(=O)COC(C)=O)CC3)CC2)n1 ; CC(C)c1nn(-c2noc(C3CCN(C4CCN(C(=O)CO)CC4)CC3)n2)c2c(F)ccc(O)c12]

The title compound was prepared in the same manner as in Example 552 except that the 4-{3-[7-fluoro-6-methoxy-3-(propan-2-yl)-1H-indazol-1-yl]-1,2,4-oxadiazol-5-yl}-1,4′-bipiperidine bis(trifluoroacetate) was replaced with 4-{3-[7-fluoro-4-methoxy-3-(propan-2-yl)-1H-indazol-1-yl]-1,2,4-oxadiazol-5-yl}-1,4′-bipiperidine bis(trifluoroacetate).

¹H-NMR (CDCl₃) δ: 1.12-1.40 (9H, m), 1.59-1.77 (4H, m), 1.93-2.06 (2H, m), 2.16-2.33 (2H, m), 2.37-2.58 (1H, m), 2.72-2.90 (3H, m), 2.97-3.10 (1H, m), 3.46-3.68 (2H, m), 3.91-4.06 (2H, m), 4.21-4.44 (2H, m), 5.65-5.69 (1H, m), 6.49 (1H, dd, J=8.5, 2.8 Hz), 7.10 (1H, dd, J=11.4, 8.4 Hz).

LC-MS, m/z; 487 [M+H]+

### Example 554

### Preparation of 1-(4-{3-[7-fluoro-6-hydroxy-3-(propan-2-yl)-1H-indazol-1-yl]-1,2,4-oxadiazol-5-yl}-1,4′-bipiperidin-1′-yl)ethanone

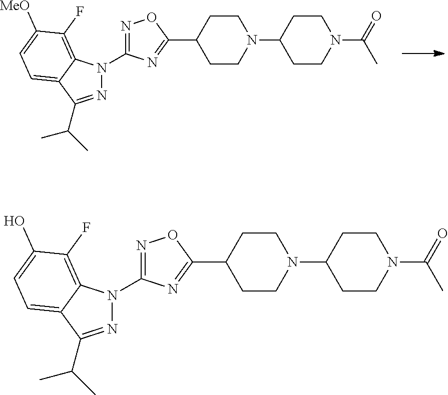 [SMILES: COc1ccc2c(C(C)C)nn(-c3noc(C4CCN(C5CCN(C(C)=O)CC5)CC4)n3)c2c1F ; CC(=O)N1CCC(N2CCC(c3nc(-n4nc(C(C)C)c5ccc(O)c(F)c54)no3)CC2)CC1]

The title compound was prepared in the same manner as in Example 552 (2) except that the intermediate of Example 552 was replaced with 1-(4-{3-[7-fluoro-6-methoxy-3-(propan-2-yl)-1H-indazol-1-yl]-1,2,4-oxadiazol-5-yl}-1,4′-bipiperidin-1′-yl)ethanone.

LC-MS, m/z; 471 [M+H]+

### Example 555

### Preparation of 1-(4-{3-[7-fluoro-4-hydroxy-3-(propan-2-yl)-1H-indazol-1-yl]-1,2,4-oxadiazol-5-yl}-1,4′-bipiperidin-1′-yl)ethanone

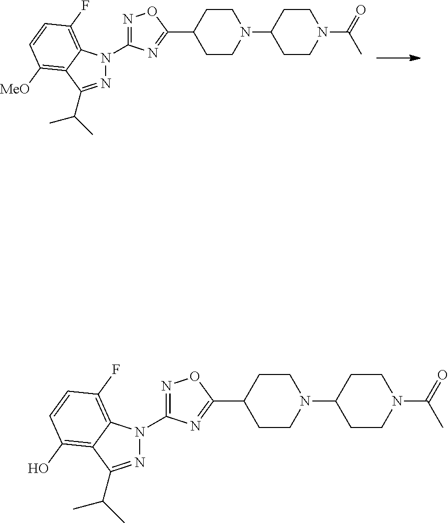 [SMILES: COc1ccc(F)c2c1c(C(C)C)nn2-c1noc(C2CCN(C3CCN(C(C)=O)CC3)CC2)n1 ; CC(=O)N1CCC(N2CCC(c3nc(-n4nc(C(C)C)c5c(O)ccc(F)c54)no3)CC2)CC1]

The title compound was prepared in the same manner as in Example 552 (2) except that the intermediate of Example 552 was replaced with 1-(4-{3-[7-fluoro-4-methoxy-3-(propan-2-yl)-1H-indazol-1-yl]-1,2,4-oxadiazol-5-yl}-1,4′-bipiperidin-1′-yl)ethanone.

LC-MS, m/z; 471 [M+H]+

### Example 556

### Preparation of ethyl 4-{3-[7-fluoro-3-(propan-2-yl)-1H-indazol-1-yl]-1,2,4-oxadiazol-5-yl}-3′-methoxy-1,4′-bipiperidine-1′-carboxylate

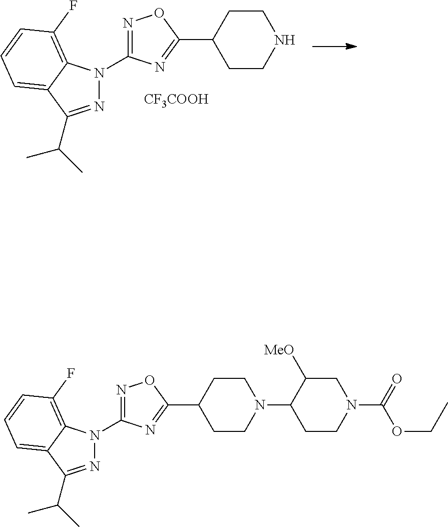 [SMILES: CC(C)c1nn(-c2noc(C3CCNCC3)n2)c2c(F)cccc12 ; "CF3COOH" ; CCOC(=O)N1CCC(N2CCC(c3nc(-n4nc(C(C)C)c5cccc(F)c54)no3)CC2)C(OC)C1]

The title compound was prepared in the same manner as in Example 028 except that the 3-ethyl-1-[5-(piperidin-4-yl)-1,2,4-oxadiazol-3-yl]-1H-indazole trifluoroacetate and (S)-(−)-1-tert-butoxycarbonyl-2-pyrrolidinecarbaldehyde were replaced with 7-fluoro-1-[5-(piperidin-4-yl)-1,2,4-oxadiazol-3-yl]-3-(propan-2-yl)-1H-indazole trifluoroacetate and ethyl 3-methoxy-4-oxopiperidine-1-carboxylate, respectively, and titanium tetraisopropoxide was added to the reaction.

¹H-NMR (CDCl₃) δ: 1.17-1.32 (3H, m), 1.50 (6H, d, J=7.0 Hz), 1.57-1.71 (1H, m), 1.85-2.23 (4H, m), 2.31-3.21 (8H, m), 3.25-3.67 (6H, m), 3.97-4.61 (4H, m), 7.15-7.28 (2H, m), 7.53-7.61 (1H, m).

LC-MS, m/z; 515 [M+H]+

### Example 557

### Preparation of potassium (4-{3-[7-fluoro-3-(propan-2-yl)-1H-indazol-1-yl]-1,2,4-oxadiazol-5-yl}-1,4′-bipiperidin-1′-yl)(oxo)acetate

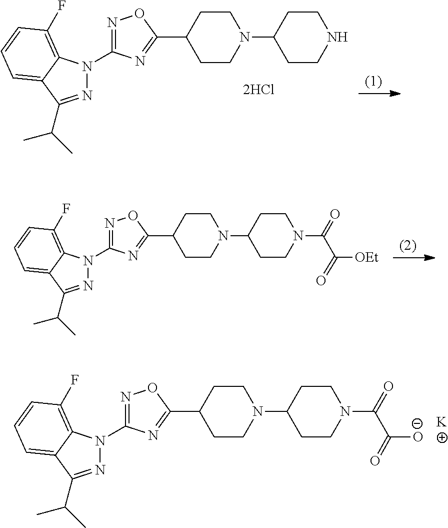 [SMILES: CC(C)c1nn(-c2noc(C3CCN(C4CCNCC4)CC3)n2)c2c(F)cccc12 ; CCOC(=O)C(=O)N1CCC(N2CCC(c3nc(-n4nc(C(C)C)c5cccc(F)c54)no3)CC2)CC1 ; CC(C)c1nn(-c2noc(C3CCN(C4CCN(C(=O)C(=O)[O-])CC4)CC3)n2)c2c(F)cccc12 ; [K+]]

(1) 4-{3-[7-Fluoro-3-(propan-2-yl)-1H-indazol-1-yl]-1,2,4-oxadiazol-5-yl}-1,4′-bipiperidine dihydrochloride (500 mg) was dissolved in dichloromethane (5.0 ml), and the solution was ice-cooled. Diisopropylethylamine (578 μl) and ethyloxalyl chloride (126 μl) were added thereto, and the mixture was stirred at the same temperature for 30 minutes. The reaction solution was diluted with chloroform and washed with water. The organic layer was dried, and the solvent was removed under reduced pressure.

(2) The residue was dissolved in methanol (5.0 ml), potassium hydroxide (58 mg) and water (1.0 ml) were added thereto, and the mixture was stirred at room temperature for 3 hours. The solvent was removed under reduced pressure, and the residue was purified by silica-gel chromatography (column; Hi-Flash™ Octadecyl, developing solvent: acetonitrile/water=1:1) to give the title compound (415 mg).

¹H-NMR (DMSO-d₆) δ: 1.11-1.46 (8H, m), 1.61-1.88 (4H, m), 2.02-2.16 (2H, m), 2.29-2.60 (4H, m), 2.73-2.97 (3H, m), 3.07-3.21 (1H, m), 3.42-3.55 (1H, m), 3.68-3.80 (1H, m), 4.19-4.30 (1H, m), 7.30-7.49 (2H, m), 7.81 (1H, d, J=7.7 Hz).

LC-MS, m/z; 485 [M+H]+

### Preparations of Examples 558 to 559

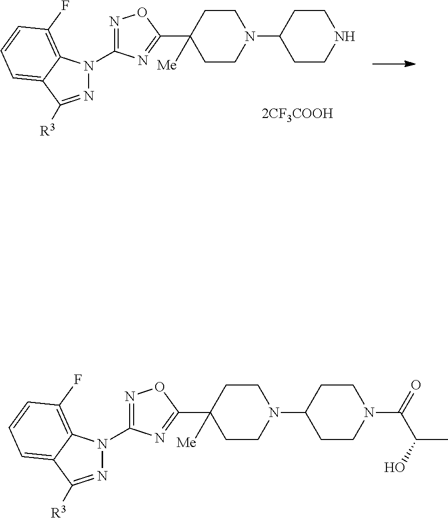

The compounds in the following table (i.e. Examples 558 to 559) were prepared in the same manner as in Example 242 except that the 4-{3-[7-fluoro-3-(propan-2-yl)-1H-indazol-1-yl]-1,2,4-oxadiazol-5-yl}-1,4′-bipiperidine dihydrochloride and acetoxyacetyl chloride were replaced with the corresponding starting compound and (S)-(−)-2-acetoxypropionyl chloride, respectively, and the 2 N sodium hydroxide aqueous solution was replaced with methylamine/methanol.

TABLE 78

| | | | ¹H-NMR/ |
|---|---|---|---|
| Ex. | R³ | Compound Name | LC-MS, m/z |
| 558 | ⁱPr | (2S)-1-(4-{3-[7- | ¹H-NMR (CDCl₃) δ: |
| | | fluoro-3-(propan- | 0.94 (3H, d, J = 5.5 Hz), |
| | | 2-yl)-1H-indazol-1-yl]-1,2,4- | 1.26-1.42 (5H, m), 1.48 (6H, |
| | | oxadiazol-5-yl}-4′- | d, J = 7.0 Hz), |
| | | methyl-1,4′- | 1.85-2.07 (4H, m), |
| | | bipiperidin-1′-yl)-2- | 2.11-2.36 (4H, m), |
| | | hydroxypropan-1-one | 2.90-3.09 (3H, m), |
| | | | 3.17-3.52 (4H, m), |
| | | | 3.89-4.13 (2H, m), |
| | | | 4.43 (1H, dd, J = 13.8, |
| | | | 7.1 Hz), |
| | | | 7.15-7.25 (2H, m), |
| | | | 7.54-7.59 (1H, m). |
| | | | LC-MS, m/z; |
| | | | 499 [M + H]+ |
| 559¹⁾ | Et | (2S)-1-{4-[3-(3- | ¹H-NMR (DMSO-d₆) δ: |
| | | ethyl-7-fluoro- | 1.15 (3H, s), |
| | | 1H-indazol-1-yl)- | 1.28-1.40 (6H, m), |
| | | 1,2,4-oxadiazol- | 1.80-2.03 (3H, m), |
| | | 5-yl]-4′-methyl-1,4′- | 2.23-2.39 (4H, m), |
| | | bipiperidin- | 2.60-2.76 (1H, m), |
| | | 1′-yl}-2-hydroxypropan-1-one | 2.95-3.18 (4H, m), |
| | | hydrochloride | 3.23-3.41 (3H, m), |
| | | | 3.46-3.72 (3H, m), |
| | | | 3.96-4.11 (1H, m), |
| | | | 4.30-4.48 (2H, m), |
| | | | 7.29-7.49 (2H, m), |
| | | | 7.70-7.79 (1H, m), |
| | | | 10.08-10.39 (1H, |
| | | | m). |
| | | | LC-MS, m/z; |
| | | | 485 [M + H]+ |

¹⁾The crude product was treated with 1N HCl/diethyl ether solution to obtain the desired compound.

### Example 560

### Preparation of 7-fluoro-1-[5-(4-fluoropiperidin-4-yl)-1,2,4-oxadiazol-3-yl]-3-(propan-2-yl)-1H-indazole trifluoroacetate

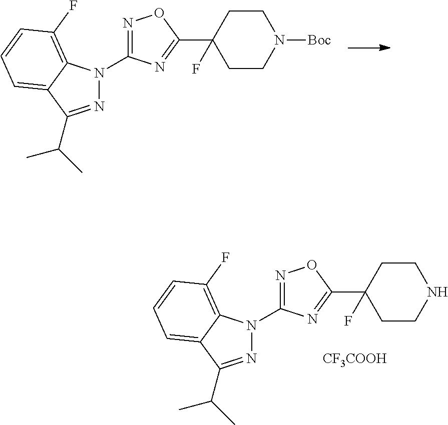 [SMILES: CC(C)c1nn(-c2noc(C3(F)CCN(C(=O)OC(C)(C)C)CC3)n2)c2c(F)cccc12 ; CC(C)c1nn(-c2noc(C3(F)CCNCC3)n2)c2c(F)cccc12 ; "CF3COOH"]

The title compound was prepared in the same manner as in Example 001 except that the tert-butyl 4-[3-(3-ethyl-6-fluoro-1H-indazol-1-yl)-1,2,4-oxadiazol-5-yl]piperidine-1-carboxylate was replaced with tert-butyl 4-fluoro-4-(3-(7-fluoro-3-isopropyl-1H-indazol-1-yl)-1,2,4-oxadiazol-5-yl)piperidine-1-carboxylate.

LC-MS, m/z; 348 [M+H]+

### Example 561

### Preparation of 1-(4-fluoro-4-{3-[7-fluoro-3-(propan-2-yl)-1H-indazol-1-yl]-1,2,4-oxadiazol-5-yl}-1,4′-bipiperidin-1′-yl)ethanone

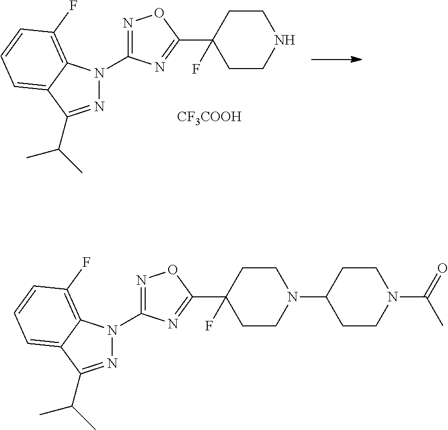 [SMILES: CC(C)c1nn(-c2noc(C3(F)CCNCC3)n2)c2c(F)cccc12 ; "CF3COOH" ; CC(=O)N1CCC(N2CCC(F)(c3nc(-n4nc(C(C)C)c5cccc(F)c54)no3)CC2)CC1]

The title compound was prepared in the same manner as in Example 028 except that the 3-ethyl-1-[5-(piperidin-4-yl)-1,2,4-oxadiazol-3-yl]-1H-indazole trifluoroacetate and (S)-(−)-1-tert-butoxycarbonyl-2-pyrrolidinecarbaldehyde were replaced with 7-fluoro-1-[5-(4-fluoropiperidin-4-yl)-1,2,4-oxadiazol-3-yl]-3-(propan-2-yl)-1H-indazole trifluoroacetate and 1-acetylpiperidin-4-one, respectively.

¹H-NMR (CDCl₃) δ: 1.39-1.57 (8H, m), 1.80-1.93 (2H, m), 2.10 (3H, s), 2.28-2.45 (4H, m), 2.51-2.89 (6H, m), 3.00-3.13 (1H, m), 3.41-3.55 (1H, m), 3.88 (1H, d, J=13.9 Hz), 4.67 (1H, d, J=13.4 Hz), 7.19-7.30 (2H, m), 7.57-7.62 (1H, m).

LC-MS, m/z; 473 [M+H]+

### Preparations of Examples 562 to 565

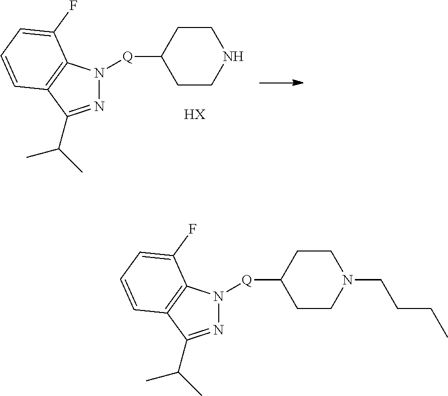

Wherein HX is hydrochloric acid or trifluoroacetic acid. HX is absent in Example 565.

The compounds in the following table (i.e. Examples 562 to 565) were prepared in the same manner as in Example 097 except that the 3-ethyl-6-fluoro-1-[5-(piperidin-4-yl)-1,2,4-oxadiazol-3-yl]-1H-indazole trifluoroacetate and ethyl iodide were replaced with the corresponding starting compound and butyl bromide, respectively. Each free form of the compounds in the following table was obtained by omitting the conversion step into hydrochloride in Example 097.

TABLE 79

| | | | ¹H-NMR/ |
|---|---|---|---|
| Ex. | Q | Compound Name | LC-MS, m/z |
| 562 | 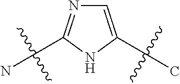 | 1-[5-(1- butylpiperidin- 4-yl)-1H- imidazol-2-yl]- 7-fluoro-3- (propan-2-yl)- 1H-indazole | LC-MS, m/z; 384 [M + H]+ |
| 563 | 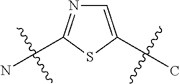 | 1-[5-(1- butylpiperidin- 4-yl)-1,3- thiazol-2-yl]-7- fluoro-3- (propan-2-yl)- 1H-indazole | ¹H-NMR (CDCl₃) δ: 0.93 (3H, t, J = 7.2 Hz), 1.26-1.41 (2H, m), 1.43- 1.58 (8H, m), 1.79-1.92 (2H, m), 1.97- 2.13 (4H, m), 2.30-2.42 (2H, m), 2.76-2.90 (1H, m), 3.03 (2H, d, J = 11.6 Hz), 3.35- 3.49 (1H, m), 7.15-7.24 (2H, m), 7.32-7.35 (1H, m), 7.49- 7.57 (1H, m). LC-MS, m/z; 401 [M + H]+ |
| 564 | 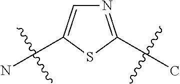 | 1-[2-(1- butylpiperidin- 4-yl)-1,3- thiazol-5-yl]-7- fluoro-3- (propan-2-yl)- 1H-indazole | ¹H-NMR (CDCl₃ ) δ: 0.94 (3H, t, J = 7.3 Hz), 1.28-1.65 (10H, m), 1.92- 2.09 (2H, m), 2.17-2.36 (4H, m), 2.41- 2.56 (2H, m), 3.00-3.20 (3H, m), 3.35- 3.50 (1H, m), 7.07-7.17 (2H, m), 7.51- 7.58 (1H, m), 7.75-7.79 (1H, m). LC-MS, m/z; 401 [M + H]+ |
| 565 | 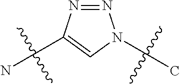 | 1-[1-(1- butylpiperidin- 4-yl)-1H- 1,2,3-triazol-4- yl]-7-fluoro-3- (propan-2-yl)- 1H-indazole | ¹H-NMR (CDCl₃) δ: 0.94 (3H, t, J = 7.2 Hz), 1.26-1.59 (10H, m), 2.04- 2.46 (8H, m), 3.03-3.17 (2H, m), 3.38-3.52 (1H, m), 4.51- 4.63 (1H, m), 7.05-7.16 (2H, m), 7.53- 7.59 (1H, m), 7.81 (1H, d, J = 1.5 Hz). LC-MS, m/z; 385 [M + H]+ |

### Example 566

### Preparation of N-{3-[(cis-3-{3-[7-fluoro-3-(propan-2-yl]-yl}cyclobutyl)amino]propyl}acetamide

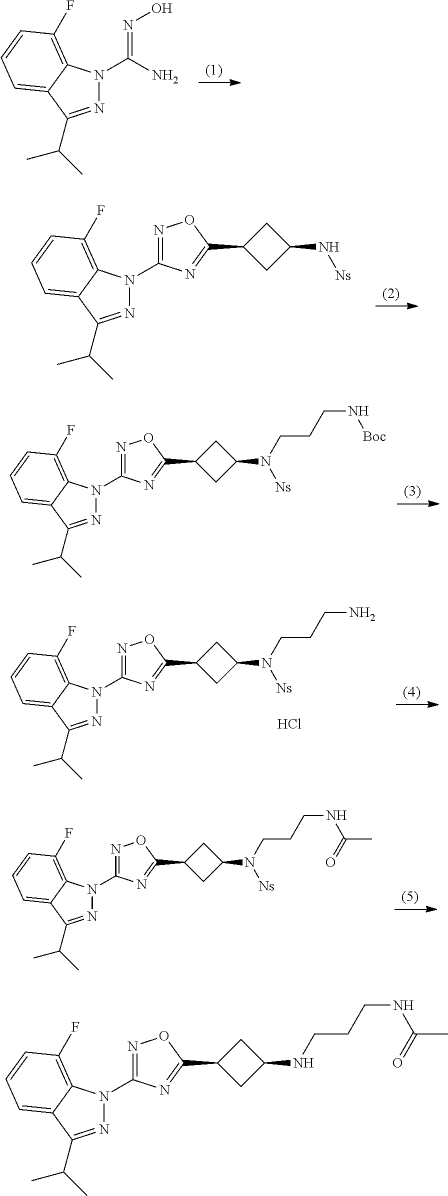

(1) N-(cis-3-{3-[7-fluoro-3-(propan-2-yl)-1H-indazol-1-yl]-1,2,4-oxadiazol-5-yl}cyclobutyl)-2-nitrobenzenesulfonamide was prepared in the same manner as in Reference Example 060 except that the 3-ethyl-6-fluoro-N-hydroxy-1H-indazole-1-carboximidamide and 3-oxocyclobutanecarboxylic acid were replaced with 7-fluoro-N′-hydroxy-3-(propan-2-yl)-1H-indazole-1-carboximidamide and cis-3-{[(2-nitrophenyl)sulfonyl]amino}cyclobutanecarboxylic acid.

(2) To the N-(cis-3-{3-[7-fluoro-3-(propan-2-yl)-1H-indazol-1-yl]-1,2,4-oxadiazol-5-yl}cyclobutyl)-2-nitrobenzenesulfonamide (200 mg), 3-(tert-butoxycarbonylamino)-1-propanol (210 mg) and tributylphosphine (0.3 ml) in THF (1 ml) was added dropwise diethyl azodicarboxylate (0.2 ml), and the mixture was stirred at 60° C. for 5 hours. To the reaction solution was added water (2 ml), and the mixture was extracted with ethyl acetate (2 ml×3). The organic layer was dried over anhydrous magnesium sulfate, filtered, and concentrated under reduced pressure. The residue was purified by silica-gel chromatography to give tert-butyl (3-{(cis-3-{3-[7-fluoro-3-(propan-2-yl)-1H-indazol-1-yl]-1,2,4-oxadiazol-5-yl}cyclobutyl)[(2-nitrophenyl)sulfonyl]amino}propyl)carbamate (170 mg).

LC-MS, m/z; 658 [M+H]+

(3) To the tert-butyl (3-{(cis-3-{3-[7-fluoro-3-(propan-2-yl)-1H-indazol-1-yl]-1,2,4-oxadiazol-5-yl}cyclobutyl) [(2-nitrophenyl)sulfonyl]amino}propyl)carbamate (170 mg) was added 4 mol/L HCl/ethyl acetate (3 ml), and the mixture was stirred at room temperature for 1 hour. The reaction solution was concentrated under reduced pressure to give a quantitative amount of N-(3-aminopropyl)-N-(cis-3-{3-[7-fluoro-3-(propan-2-yl)-1H-indazol-1-yl]-1,2,4-oxadiazol-5-yl}cyclobutyl)-2-nitrobenzenesulfonamide hydrochrolide.

LC-MS, m/z; 558 [M+H]+

(4) To the N-(3-aminopropyl)-N-(cis-3-{3-[7-fluoro-3-(propan-2-yl)-1H-indazol-1-yl]-1,2,4-oxadiazol-5-yl}cyclobutyl)-2-nitrobenzenesulfonamide hydrochrolide (60 mg) and triethylamine (30 μl) in dichloromethane (1 ml) was added acetyl chloride (9 μl), and the mixture was stirred at room temperature for 1 hour. The reaction solution was purified by silica-gel chromatography to give N-(3-{(cis-3-{3-[7-fluoro-3-(propan-2-yl)-1H-indazol-1-yl]-1,2,4-oxadiazol-5-yl}cyclobutyl) [(2-nitrophenyl)sulfonyl]amino}propyl)acetamide (56 mg).

(5) To the N-(3-{(cis-3-{3-[7-fluoro-3-(propan-2-yl)-1,4-indazol-1-yl]-1,2,4-oxadiazol-5-yl}cyclobutyl) [(2-nitrophenyl)sulfonyl]amino}propyl)acetamide (56 mg) and cesium carbonate (120 mg) in acetonitrile (1 ml) was added thioglycolic acid (34 μl), and the mixture was stirred at 60° C. for 4 hours. To the reaction solution was added water, and the mixture was extracted with ethyl acetate (1 ml×3). The organic layer was concentrated under reduced pressure and the residue was purified by silica-gel chromatography to give the title compound (12 mg).

¹H-NMR (CDCl₃) δ: 1.50 (6H, d, J=7.0 Hz), 1.63-1.75 (2H, m), 1.97 (3H, s), 2.24-2.39 (2H, m), 2.69 (2H, t, J=6.3 Hz), 2.78-2.90 (2H, m), 3.27-3.59 (6H, m), 6.33-6.46 (1H, m), 7.18-7.28 (2H, m), 7.56-7.63 (1H, m).

LC-MS, m/z; 415 [M+H]+

The compounds in the following table (i.e. Examples 567 to 568) were prepared in the same manner as in Example 028 except that the 3-ethyl-1-[5-(piperidin-4-yl)-1,2,4-oxadiazol-3-yl]-1,1-indazole trifluoroacetate and (S)-(−)-1-tert-butoxycarbonyl-2-pyrrolidine-carbaldehyde were replaced with the corresponding starting compound and 1-acetylpiperidin-4-one, respectively.

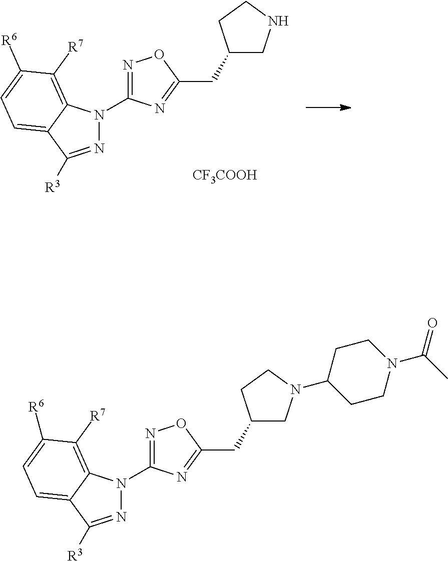

TABLE 80

| Example | R³ | R⁶ | R⁷ | Compound Name | ¹H-NMR/LC-MS, m/z |
|---|---|---|---|---|---|
| 567¹⁾ | Et | H | F | 1-{4-[(3S)-3- | ¹H-NMR (CDCl₃) δ: |
| | | | | {[3-(3-ethyl-7- | 1.34-1.54 (5H, m), 1.59-1.72 (1H, m), |
| | | | | fluoro-1H- | 1.80-1.94 (2H, m), |
| | | | | indazol-1-yl)- | 2.03-2.33 (5H, m), |
| | | | | 1,2,4-oxadiazol- | 2.41-2.52 (1H, m), 2.57-2.97 (5H, m), |
| | | | | 5- | 3.01-3.16 (5H, m), |
| | | | | yl]methyl}pyrrolidin- | 3.70-3.81 (1H, m), |
| | | | | 1- | 4.32-4.44 (1H, m), 7.19-7.31 (2H, m), |
| | | | | yl]piperidin-1- | 7.50-7.57 (1H, m). |
| | | | | yl}ethanone | LC-MS, m/z; 441 [M + H]+ |
| 568¹⁾ | ⁱPr | F | H | 1-{4-[(3S)-3- | ¹H-NMR (CDCl₃) δ: |
| | | | | ({3-[6-fluoro-3- | 1.34-1.56 (8H, m), 1.58-1.73 (1H, m), |
| | | | | (propan-2-yl)- | 1.80-1.95 (2H, m), |
| | | | | 1H-indazol-1- | 2.05-2.51 (6H, m), |
| | | | | yl]-1,2,4- | 2.60-2.96 (5H, m), 3.04-3.16 (3H, m), |
| | | | | oxadiazol-5- | 3.41-3.54 (1H, m), |
| | | | | yl}methyl)pyrrolidin- | 3.77 (1H, d, J = 13.6 Hz), |
| | | | | 1- | 4.38 (1H, d, J = 13.4 Hz), |
| | | | | yl]piperidin-1- | 7.07 (1H, td, J = 8.8, 2.3 Hz), |
| | | | | yl}ethanone | 7.76 (1H, dd, J = 8.8, 5.0 Hz), |
| | | | | | 7.98 (1H, dd, J = 9.4, |
| | | | | | 2.2 Hz). |
| | | | | | LC-MS, m/z; 455 [M + H]+ |

¹⁾Titanium tetraisopropoxide was added to the reaction system.

### Preparations of Examples 569 to 572

The compounds in the following table (i.e. Examples 569 to 572) were prepared in the same manner as in Example 168 except that the 3-ethyl-1-(5-{1-[2-(piperidin-4-yl)ethyl]piperidin-4-yl}-1,2,4-oxadiazol-3-yl)-1H-indazole bis(trifluoroacetate) and methyl chloroformate were replaced with the corresponding starting compound and 2-methoxyacetyl chloride, respectively.

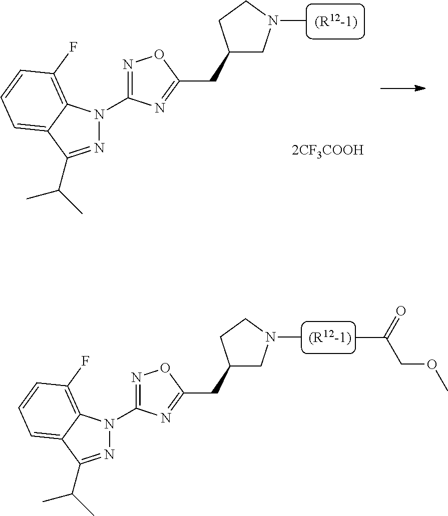

Wherein (R¹²-1) means each cyclic amino structure shown in the following table; and the methoxyacetyl group is attached to the nitrogen atom in the cyclic amine of (R¹²-1). Each free form of the compounds in the following table was obtained by omitting the conversion step into hydrochloride in Example 168.

TABLE 81

| Exam- | | Chemical | ¹H-NMR/ |
|---|---|---|---|
| ple | (R¹²-1) | Name | LC-MS, m/z |
| 569 | 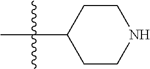 [SMILES: *C1CCNCC1] | 1-{4-[(3R)-3- ({3-[7-fluoro- 3-(propan-2- yl)-1H-indazol- 1-yl]-1,2,4- oxadiazol-5- yl}methyl) pyrrolidin-1- yl]piperidin- 1-yl}-2- methoxy- ethanone | ¹H-NMR (CDCl₃) δ: 1.38-1.56 (8H, m), 1.59-1.73 (1H, m), 1.83-1.95 (2H, m), 2.09-2.34 (2H, m), 2.41-2.52 (1H, m), 2.59-2.95 (5H, m), 3.00-3.14 (3H, m), 3.39-3.55 (4H, m), 3.83 (1H, d, J = 13.2 Hz), 4.01-4.18 (2H, m), 4.36 (1H, d, J = 11.4 Hz), 7.18-7.28 (2H, m), 7.56-7.62 (1H, m). LC-MS, m/z; 485 [M + H]+ |
| 570 | 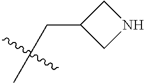 [SMILES: *CC1CNC1] | 1-(3-{[(3R)- 3-({3-[7- fluoro-3- (propan-2- yl)-1H-indazol- 1-yl]-1,2,4- oxadiazol-5- yl}methyl) pyrrolidin-1- yl]methyl} azetidin-1- yl)-2- methoxy- ethanone | ¹H-NMR (CDCl₃) δ: 1.50 (6H, d, J = 7.2 Hz), 1.56- 1.70 (1H, m), 2.07-2.22 (1H, m), 2.36-2.87 (8H, m), 3.06 (2H, d, J = 6.8 Hz), 3.33-3.56 (4H, m), 3.70 (1H, dd, J = 9.9, 5.1 Hz), 3.85-3.99 (3H, m), 4.08-4.19 (1H, m), 4.25-4.36 (1H, m), 7.19-7.29 (2H, m), 7.56-7.62 (1H, m). LC-MS, m/z; 471 [M + H]+ |
| 571 | 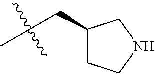 [SMILES: *C[C@@H]1CCNC1] | 1-[(3S)-3- {[(3R)-3- ({3-[7- fluoro-3- (propan-2- yl)-1H-indazol- 1-yl]-1,2,4- oxadiazol- 5-yl}methyl) pyrrolidin-1- yl]methyl} pyrrolidin- 1-yl]-2- methoxy- ethanone | ¹H-NMR (CDCl₃) δ: 1.44-1.79 (8H, m), 1.93-2.37 (10H, m), 3.02- 3.22 (3H, m), 3.35-3.78 (7H, m), 4.03 (2H, s), 7.18-7.29 (2H, m), 7.57-7.63 (1H, m). LC-MS, m/z; 485 [M + H]+ |
| 572 | 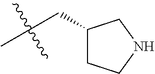 [SMILES: *C[C@H]1CCNC1] | 1-[(3R)-3- {[(3R)-3- ({3-[7- fluoro-3- (propan-2- yl)-1H- indazol-1- yl]-1,2,4- oxadiazol-5- yl}methyl) pyrrolidin-1- yl]methyl} pyrrolidin-1- yl]-2- methoxy- ethanone | ¹H-NMR (CDCl₃) δ: 1.46-1.79 (8H, m), 2.05-2.21 (2H, m), 2.28-2.83 (8H, m), 3.03-3.20 (3H, m), 3.35-3.78 (7H, m), 3.99-4.04 (2H, m), 7.19-7.29 (2H, m), 7.57-7.62 (1H, m). LC-MS, m/z; 485 [M + H]+ |

### Preparations of Examples 573 to 576

The compounds in the following table (i.e. Examples 573 to 576) were prepared in the same manner as in Example 168 except that the 3-ethyl-1-(5-{1-[2-(piperidin-4-yl)ethyl]piperidin-4-yl}-1,2,4-oxadiazol-3-yl)-1H-indazole bis(trifluoroacetate) and methyl chloroformate were replaced with the corresponding starting compound and 2-methoxyacetyl chloride, respectively.

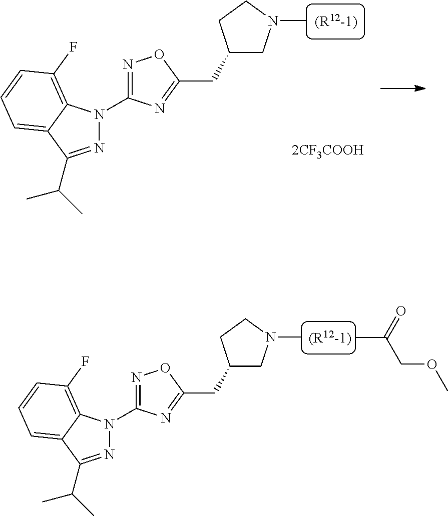

Wherein (R¹²-1) means each cyclic amino structure shown in the following table; and the methoxyacetyl group is attached to the nitrogen atom in the cyclic amine of (R¹²-1). Each free form of the compounds in the following table was obtained by omitting the conversion step into hydrochloride in Example 168.

TABLE 82

| Exam- | | Compound | ¹H-NMR/ |
|---|---|---|---|
| ple | (R¹²-1) | Name | LC-MS, m/z |
| 573 | 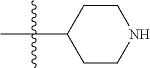 [SMILES: *C1CCNCC1] | 1-{4-[(3S)- 3-({3-[7- fluoro-3- (propan-2- yl)-1H- indazol-1- yl]-1,2,4- oxadiazol-5- yl}methyl) pyrrolidin-1- yl]piperidin- 1-yl}-2- methoxy- ethanone | ¹H-NMR (CDCl₃) δ: 1.38-1.56 (8H, m), 1.59-1.73 (1H, m), 1.83-1.95 (2H, m), 2.09-2.34 (2H, m), 2.41-2.52 (1H, m), 2.59-2.95 (5H, m), 3.00-3.14 (3H, m), 3.39-3.55 (4H, m), 3.83 (1H, d, J = 13.2 Hz), 4.01- 4.18 (2H, m), 4.36 (1H, d, J = 11.4 Hz), 7.18-7.28 (2H, m), 7.56-7.62 (1H, m). LC-MS, m/z; 485 [M + H]+ |
| 574 | 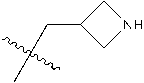 [SMILES: *CC1CNC1] | 1-(3-{[(3S)- 3-({3-[7- fluoro-3- (propan-2- yl)-1H- indazol-1- yl]-1,2,4- oxadiazol-5- yl}methyl) pyrrolidin-1- yl]methyl} azetidin-1- yl)-2- methoxy- ethanone | 1H-NMR (CDCl₃) δ: 1.50 (6H, d, J = 7.2 Hz), 1.56-1.70 (1H, m), 2.07-2.22 (1H, m), 2.36-2.87 (8H, m), 3.06 (2H, d, J = 6.8 Hz), 3.33-3.56 (4H, m), 3.70 (1H, dd, J = 9.9, 5.1 Hz), 3.85- 3.99 (3H, m), 4.08- 4.19 (1H, m), 4.25- 4.36 (1H, m), 7.19- 7.29 (2H, m), 7.56- 7.62 (1H, m). LC-MS, m/z; 471 [M + H]+ |
| 575 | 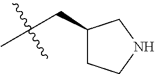 [SMILES: *C[C@@H]1CCNC1] | 1-[(3S)-3- {[(3S)-3- ({3-[7- fluoro-3- (propan-2- yl)-1H- indazol-1- yl]-1,2,4- oxadiazol-5- yl}methyl) pyrrolidin-1- yl]methyl} pyrrolidin-1- yl]-2- methoxy- ethanone | ¹H-NMR (CDCl₃) δ: 1.46-1.79 (8H, m), 2.05-2.21 (2H, m), 2.28-2.83 (8H, m), 3.03-3.20 (3H, m), 3.35-3.78 (7H, m), 3.99-4.04 (2H, m), 7.19-7.29 (2H, m), 7.57-7.62 (1H, m). LC-MS, m/z; 485 [M + H]+ |
| 576 | 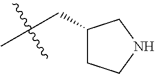 [SMILES: *C[C@H]1CCNC1] | 1-[(3R)-3- {[(3S)-3- ({3-[7- fluoro-3- (propan-2- yl)-1H- indazol-1- yl]-1,2,4- oxadiazol-5- yl}methyl) pyrrolidin-1- yl]methyl} pyrrolidin-1- yl]-2- methoxy- ethanone | ¹H-NMR (CDCl₃) δ: 1.44-1.79 (8H, m), 1.93-2.87 (10H, m), 3.02-3.22 (3H, m), 3.35-3.78 (7H, m), 4.03 (2H, s), 7.18-7.29 (2H, m), 7.57-7.63 (1H, m). LC-MS, m/z; 485 [M + H]+ |

The compounds in the following table (i.e. Examples 577 to 579) were prepared in the same manner as in Example 001 except that the tert-butyl 4-[3-(3-ethyl-6-fluoro-1H-indazol-1-yl)-1,2,4-oxadiazol-5-yl]piperidin-1-carboxylate was replaced with the corresponding starting compound (see, Reference Examples 147 to 149).

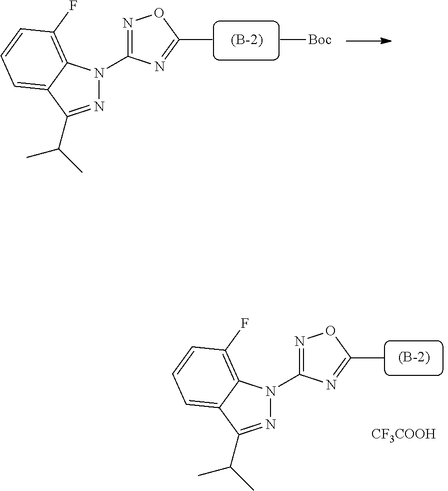

TABLE 83

| Exam- | | Compound | ¹H-NMR/ |
|---|---|---|---|
| ple | (B-2) | Name | LC-MS, m/z |
| 577 | 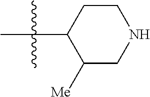 [SMILES: *C1CCNCC1C] | 7-fluoro-1- [5-(3-methyl- piperidin-4- yl)-1,2,4- oxadiazol-3- yl]-3-(propan- 2-yl)-1H- indazole trifluoro- acetate | LC-MS, m/z; 344 [M + H]+ |
| 578 | 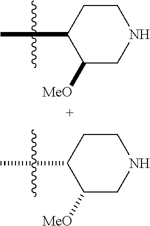 | 7-fluoro-1- {5-[(3S,4S)-3- methoxy- piperidin-4- yl]-1,2,4- oxadiazol-3- yl}-3-(propan- 2-yl)-1H- indazole trifluoro- acetate and 7-fluoro-1- {5-[(3R,4R)- 3-methoxy- piperidin-4- yl]-1,2,4- oxadiazol-3- yl}-3-(propan- 2-yl)-1H- indazole trifluoro- acetate | LC-MS, m/z; 360 [M + H]+ ¹H-NMR (CDCl₃) δ: 1.48 (6H, d, J = 7.0 Hz), 2.31- 2.75 (2H, m), 3.12-3.56 (7H, m), 3.61-3.84 (2H, m), 4.24 (1H, s), 7.18- 7.29 (2H, m), 7.58 (1H, d, J = 7.7 Hz), 9.46-9.95 (1H, m). |
| 579 | 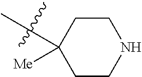 [SMILES: *C1(C)CCNCC1] | 7-fluoro-1- [5-(4-methyl- piperidin-4- yl)-1,2,4- oxadiazol-3- yl]-3-(propan- 2-yl)-1H- indazole trifluoro- acetate | LC-MS, m/z; 344 [M + H]+ |

The compounds in the following table (i.e. Examples 580 to 581) were prepared in the same manner as in Example 028 except that the 3-ethyl-1-[5-(piperidin-4-yl)-1,2,4-oxadiazol-3-yl]-1H-indazole trifluoroacetate and (S)-(−)-1-tert-butoxycarbonyl-2-pyrrolidine-carbaldehyde were replaced with the corresponding starting compound and 1-acetylpiperidin-4-one, respectively.

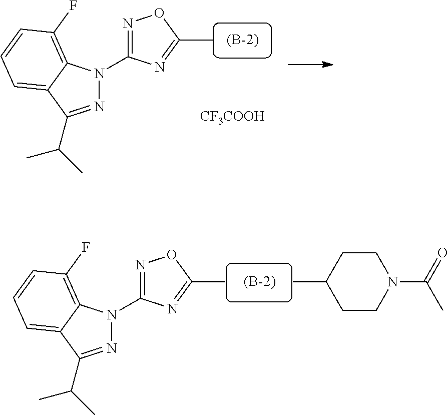

Wherein (B-2) means each cyclic amino structure shown in the following table, and the N-acetylpiperidyl group is attached to the nitrogen atom in the cyclic amine of (B-2).

TABLE 84

| Exam- | | Compound | ¹H-NMR/ |
|---|---|---|---|
| ple | (B-2) | Name | LC-MS, m/z |
| 580¹⁾ | 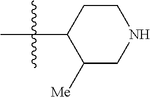 [SMILES: *C1CCNCC1C] | 1-(4-{3- [7-fluoro-3- (propan-2- yl)-1H- indazol-1- yl]-1,2,4- oxadiazol-5- yl}-3- methyl-1,4′- bipiperidin- 1′-yl) ethanone | LC-MS, m/z; 469 [M + H]+ |
| 581¹⁾ | 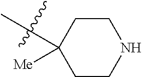 [SMILES: *C1(C)CCNCC1] | 1-(4-{3-[7- fluoro-3- (propan-2- yl)-1H- indazol-1- yl]-1,2,4- oxadiazol-5- yl}-4- methyl-1,4′- bipiperidin- 1′-yl) ethanone | ¹H-NMR (CDCl₃) δ: 1.34-1.57 (11H, m), 1.75-1.91 (4H, m), 2.08 (3H, s), 2.35-2.59 (6H, m), 2.74-2.87 (2H, m), 2.95-3.09 (1H, m), 3.42-3.55 (1H, m), 3.84 (1H, d, J = 11.2 Hz), 4.63 (1H, d, J = 13.2 Hz), 7.16-7.28 (2H, m), 7.56-7.63 (1H, m). LC-MS, m/z; 469 [M + H]+ |

¹⁾Titanium tetraisopropoxide was added to the reaction system.

### Preparations of Examples 582 to 584

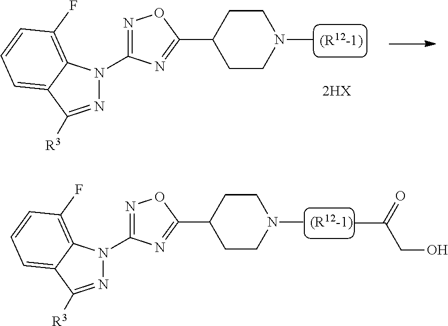

Wherein HX means hydrochloric acid or trifluoroacetic acid; (R¹²-1) means each cyclic amino structure shown in the following table; and the hydroxyacetyl group is attached to the nitrogen atom in the cyclic amine of (R¹²-1).

The compounds in the following table (i.e. Examples 582 to 584) were prepared in the same manner as in Example 242 or the 2 N sodium hydroxide aqueous solution was replaced with methyl amine/methanol, provided that the 4-{3-[7-fluoro-3-(propan-2-yl)-1H-indazol-1-yl]-1,2,4-oxadiazol-5-yl}-1,4′-bipiperidine dihydrochloride was replaced with the corresponding starting compound. Each hydrochloride in the following table was obtained by treating a solution of the prepared compound in dichloromethane with 1N HCl/diethyl ether.

TABLE 85

| Exam- | | | Compound | ¹H-NMR/ |
|---|---|---|---|---|
| ple | R³ | (R¹²-1) | Name | LC-MS, m/z |
| 582 | Et | 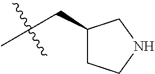 [SMILES: *C[C@@H]1CCNC1] | 1-[(3S)-3- ({4-[3- (3-ethyl- 7-fluoro- 1H-indazol- 1-yl)-1,2,4- oxadiazol-5- yl]piperidin- 1-yl}methyl) pyrrolidin-1- yl]-2- hydroxy- ethanone | ¹H-NMR (CDCl₃) δ: 1.44 (3H, t, J = 7.6 Hz), 1.50-1.85 (2H, m), 1.96-2.62 (10H, m), 2.83-3.13 (3H, m), 3.03 (2H, q, J = 7.6 Hz), 3.18-3.57 (3H, m), 3.63-3.30 (1H, m), 4.02-4.13 (2H, m), 7.20-7.31 (2H, m), 7.50-7.57 (1H, m). LC-MS, m/z; 457 [M + H]+ |
| 583 | Et | 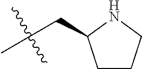 [SMILES: *C[C@@H]1CCCN1] | 1-[(2S)-2- ({4-[3-(3- ethyl-7- f luoro-1H- indazol-1- yl)-1,2,4- oxadiazol-5- yl]piperidin- 1-yl}methyl) pyrrolidin-1- yl]-2- hydroxy- ethanone | ¹H-NMR (CDCl₃) δ: 1.44 (3H, t, J = 7.5 Hz), 1.80- 2.40 (11.3H, m, rotamer), 2.61-2.69 (0.7H, m, rotamer), 2.80-3.29 (6H, m), 3.29-3.38 (0.7H, m, rotamer), 3.45-3.55 (1H, m), 3.60-3.70 (0.3H, m, rotamer), 3.81-3.90 (0.3H, m, rotamer), 4.00-4.11 (1.4H, m, rotamer), 4 .12-4.38 (1.3H, m, rotamer), 7.18-7.30 (2H, m), 7.48-7.56 (1H, m). LC-MS, m/z; 457 [M + H]+ |
| 584 | ⁱPr | 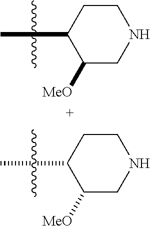 | 1-[(3S,4S)-4- {3-[7-fluoro- 3-(propan-2- yl)-1H- indazol-1- yl]-1,2,4- oxadiazol-5- yl}-3- methoxy-1,4′- bipiperidin- 1′-yl]-2- hydroxy- ethanone hydro- chloride and 1-((3R, 4R)-4-{3- [7-fluoro-3- (propan-2- yl)-1H- indazol-1- yl]-1,2,4- oxadiazol-5- yl}-3- methoxy-1,4′- bipiperidin- 1′-yl]-2- hydroxy- ethanone hydro- chloride | ¹H-NMR (CD₃OD) δ: 1.08-1.23 (2H, m), 1.47 (6H, d, J = 5.9 Hz), 1.60-1.94 (2H, m), 2.07-2.35 (2H, m), 2.38-2.72 (2H, m), 2.72-2.87 (1H, m), 3.08-3.53 (7H, m), 3.56-3.76 (3H, m), 3.84-4.07 (2H, m), 4.14-4.50 (2H, m), 4.63-4.77 (1H, m), 7.26-7.40 (2H, m), 7.67-7.80 (1H, m). LC-MS, m/z; 501 [M + H]+ |

### Preparation of Examples 585-589

The compounds in the following table (i.e. Examples 585 to 589) were prepared in the same manner as in Example 168 except that the 3-ethyl-1-(5-{1-[2-(piperidin-4-yl)ethyl]piperidin-4-yl}-1,2,4-oxadiazol-3-yl)-1H-indazole bis(trifluoroacetate) and methyl chloroformate were replaced with the corresponding starting compound, and acid chloride (R—Cl) or acetic anhydride, respectively.

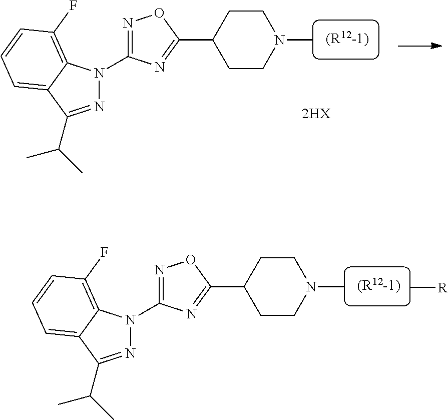

Wherein HX means hydrochloric acid or trifluoroacetic acid; (R¹²-1) means each cyclic amino structure shown in the following table; R means each structure shown in the following table; and R is attached to the nitrogen atom in the cyclic amine of (R¹²-1). Each free form of the compounds in the following table was obtained by omitting the conversion step into hydrochloride in Example 168.

TABLE 86

| Example | (R¹²-1) | R | Compound Name | ¹H-NMR/LC-MS, m/z |
|---|---|---|---|---|
| 585 | 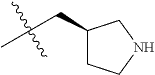 [SMILES: *C[C@@H]1CCNC1] | —Ms | 7-fluoro-1- [5-(1-{[(3S)- 1-(methyl- sulfonyl) pyrrolidin-3- yl]methyl} piperidin-4- yl)-1,2,4- oxadiazol-3- yl]-3- (propan-2- yl)-1H- indazole | ¹H-NMR (CDCl₃) δ: 1.50 (6H, d, J = 6.8 Hz), 1.63-1.77 (1H, m), 1.96- 2.26 (7H, m), 2.29-2.43 (2H, m), 2.43-2.58 (1H, m), 2.84 (3H, s), 2.86- 3.13 (4H, m), 3.28-3.38 (1H, m), 3.39-3.53 (3H, m), 7.18-7.28 (2H, m), 7.55-7.63 (1H, m). LC-MS, m/z; 491 [M + H]+ |
| 586 | 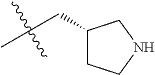 [SMILES: *C[C@H]1CCNC1] | —Ms | 7-fluoro-1- [5-(1-{[(3R)- 1-(methyl- sulfonyl) pyrrolidin-3- yl]methyl} piperidin-4- yl)-1,2,4- oxadiazol-3- yl]-3- (propan-2- yl)-1H- indazole | ¹H-NMR (CDCl₃) δ: 1.50 (6H, d, J = 6.8 Hz), 1.63-1.77 (1H, m), 1.96- 2.26 (7H, m), 2.29-2.43 (2H, m), 2.43-2.58 (1H, m), 2.84 (3H, s), 2.86- 3.13 (4H, m), 3.28-3.38 (1H, m), 3.39-3.53 (3H, m), 7.18-7.28 (2H, m), 7.55-7.63 (1H, m). LC-MS, m/z; 491 [M + H]+ |
| 587 | 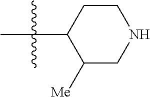 [SMILES: *C1CCNCC1C] | —Ac | 1-(4-{3-[7- fluoro-3- (propan-2- yl)-1H- indazol-1- yl]-1,2,4- oxadiazol-5- yl}-3′- methyl-1,4′- bipiperidin- 1′-yl)ethanone | ¹H-NMR (CDCl₃) δ: 0.88- 0.97 (3H, m), 1.50 (6H, d, J = 7.2 Hz), 1.73-1.89 (2H, m), 1.98-2.30 (11H, m), 2.40-2.65 (1H, m), 2.96-3.21 (4H, m), 3.41- 3.56 (1H, m), 3.62-3.92 (1H, m), 4.51-4.78 (1H, m), 7.18-7.30 (2H, m), 7.56-7.64 (1H, m). LC-MS, m/z; 469 [M + H]+ |
| 588 | 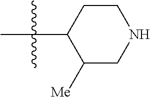 [SMILES: *C1CCNCC1C] | 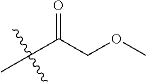 [SMILES: *C(=O)COC] | 1-(4-{3-[7- fluoro-3- (propan-2- yl)-1H- indazol-1- yl]-1,2,4- oxadiazol-5- yl}-3′- methyl-1,4′- bipiperidin- 1′-yl)-2- methoxy- ethanone | ¹H-NMR (CDCl₃) δ: 0.84- 0.99 (3H, m), 1.50 (6H, d, J = 7.0 Hz), 1.75-1.88 (2H, m), 1.93-2.30 (7H, m), 2.42-2.74 (1H, m), 2.87-3.22 (4H, m), 3.38- 3.57 (5H, m), 3.67-4.01 (1H, m), 4.04-4.28 (2H, m), 4.45-4.77 (1H, m), 7.18-7.32 (2H, m), 7.55- 7.63 (1H, m). LC-MS, m/z; 499 [M + H]+ |
| 589 | | —Ms | 4-{3-[7- fluoro-3- (propan-2- yl)-1H- indazol-1- yl]-1,2,4- oxadiazol-5- yl}-3′- methyl-1′- (methyl- sulfonyl)-1,4′- bipiperidine | ¹H-NMR (CDCl₃) δ: 1.04 (3H, d, J = 6.8 Hz), 1.40-1.76 (8H, m), 1.79- 1.91 (1H, m), 1.96-2.30 (8H, m), 2.50-2.88 (5H, m), 2.97-3.17 (2H, m), 3.39-3.54 (1H, m), 3.56- 3.71 (1H, m), 3.80-3.90 (1H, m), 7.15-7.26 (2H, m), 7.54-7.61 (1H, m). LC-MS, m/z; 505 [M + H]+ |

### Preparations of Examples 590-610

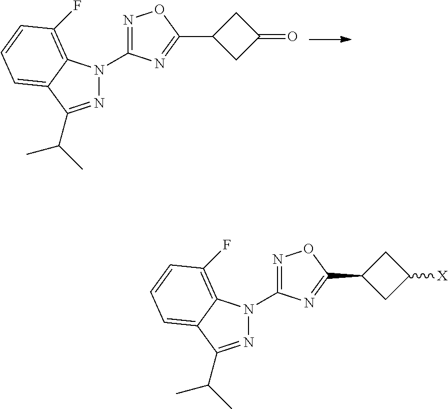

Wherein X means each structure shown in the following table.

The compounds in the following table (i.e. Examples 590 to 610) were prepared in the same manner as in Example 272 except that the 4-[3-(3-ethyl-1H-indazol-1-yl)-1,2,4-oxadiazol-5-yl]cyclohexanone and morpholine were replaced with 3-{3-[7-fluoro-3-(propan-2-yl)-1H-indazol-1-yl]-1,2,4-oxadiazol-5-yl}cyclobutanone and the corresponding amine, respectively.

TABLE 87

| Example | X | Compound Name | ¹H-NMR/LC-MS, m/z |
|---|---|---|---|
| 590 | 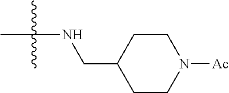 [SMILES: *NCC1CCN(C(C)=O)CC1] | 1-(4-{[(cis-3-{3- [7-fluoro-3- (propan-2-yl)-1H- indazol-1-yl]- 1,2,4-oxadiazol-5- yl}cyclobutyl) amino]methyl] piperidin-1-yl) ethanone | ¹H-NMR (CDCl₃) δ: 0.99-1.23 (2H, m), 1.47 (6H, d, J = 7.0 Hz), 1.56-1.89 (3H, m), 2.06 (3H, s), 2.20-2.33 (2H, m), 2.40-2.59 (3H, m), 2.69-2.88 (2H, m), 2.94-3.10 (1H, m), 3.28-3.55 (3H, m), 3.79 (1H, d, J = 13.9 Hz), 4.60 (1H, d, J = 13.9 Hz), 7.14-7.26 (2H, m), 7.52-7.60 (1H, m). LC-MS, m/z; 455 [M + H]+ |
| 591 | 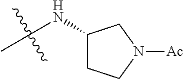 [SMILES: *N[C@H]1CCN(C(C)=O)C1] | 1-{(3S)-3-[(3-{3- [7-fluoro-3- (propan-2-yl)-1H- indazol-1-yl]- 1,2,4-oxadiazol-5- yl}cyclobutyl) amino]pyrrolidin- 1-yl}ethanone | ¹H-NMR (CDCl₃) δ: 1.47 (6H, dd, J = 7.1, 0.8 Hz), 1.65-2.21 (6H, m), 2.27-2.50 (2H, m), 2.78-2.93 (2H, m), 3.16-3.71 (7H, m), 7.16-7.26 (2H, m), 7.53-7.59 (1H, m). LC-MS, m/z; 427 [M + H]+ |
| 592 | 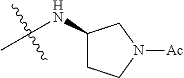 [SMILES: *N[C@@H]1CCN(C(C)=O)C1] | 1-{(3R)-3-[(3-{3- [7-fluoro-3- (propan-2-yl)-1H- indazol-1-yl]- 1,2,4-oxadiazol-5- yl}cyclobutyl) amino]pyrrolidin- 1-yl}ethanone | ¹H-NMR (CDCl₃) δ: 1.47 (6H, dd, J = 7.1, 0.8 Hz), 1.65-2.21 (6H, m), 2.27-2.50 (2H, m), 2.78-2.93 (2H, m), 3.16-3.71 (7H, m), 7.16-7.26 (2H, m), 7.53-7.59 (1H, m). LC-MS, m/z; 427 [M + H]+ |
| 593 | 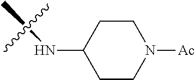 | 1-{4-[(cis-3-{3- [7-fluoro-3- (propan-2-yl)-1H- indazol-1-yl]- 1,2,4-oxadiazol-5- yl}cyclobutyl) amino]piperidin-1- yl}ethanone | ¹H-NMR (CDCl₃) δ: 1.18-1.36 (2H, m), 1.46 (6H, d, J = 7.0 Hz), 1.77-1.92 (2H, m), 2.04-2.07 (3H, m), 2.22-2.37 (2H, m), 2.63-2.91 (4H, m), 3.00-3.15 (1H, m), 3.37-3.55 (3H, m), 3.77 (1H, d, J = 13.8 Hz), 4.43 (1H, d, J = 13.4 Hz), 7.14-7.26 (2H, m), 7.54-7.59 (1H, m). LC-MS, m/z; 441 [M + H]+ |
| 594 | 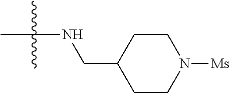 [SMILES: *NCC1CCN(S(C)(=O)=O)CC1] | cis-3-{3-[7- fluoro-3-(propan- 2-yl)-1H-indazol- 1-yl]-1,2,4- oxadiazol-5-yl}-N- {[1- (methylsulfonyl) piperidin-4- yl]methyl} cyclobutanamine | ¹H-NMR (CDCl₃) δ: 1.16- 1.21 (1H, m), 1.30 (2H, ddd, J = 25.1, 12.6, 4.6 Hz), 1.43-1.59 (7H, m), 1.66-1.93 (3H, m), 2.20-2.35 (1H, m), 2.50 (2H, d, J = 6.6 Hz), 2.62 (2H, td, J = 11.9, 2.4 Hz), 2.71-2.88 (4H, m), 3.27-3.54 (3H, m), 3.80 (2H, d, J = 11.7 Hz), 7.15-7.26 (2H, m), 7.54-7.60 (1H, m). LC-MS, m/z; 491 [M + H]+ |
| 595 | 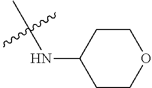 [SMILES: *NC1CCOCC1] | N-(3-{3-[7-fluoro- 3-(propan-2-yl)- 1H-indazol-1-yl]- 1,2,4-oxadiazol-5- yl}cyclobutyl) tetrahydro-2H- pyran-4-amine | LC-MS, m/z; 400 [M + H]+ |
| 596 | 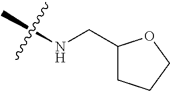 | cis-3-{3-[7- fluoro-3-(propan- 2-yl)-1H-indazol- 1-yl]-1,2,4- oxadiazol-5-yl}-N- (tetrahydrofuran- 2-ylmethyl) cyclobutanamine | LC-MS, m/z; 400 [M + H]+ |
| 597 | 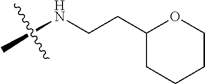 | cis-3-{3-[7- fluoro-3-(propan- 2-yl)-1H-indazol- 1-yl]-1,2,4- oxadiazol-5-yl}-N- [2-(tetrahydro-2H- pyran-2- yl)ethyl] cyclobutanamine | LC-MS, m/z; 428 [M + H]+ |
| 598 | 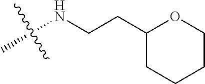 | trans-3-{3-[7- fluoro-3-(propan- 2-yl)-1H-indazol- 1-yl]-1,2,4- oxadiazol-5-yl}-N- [2-(tetrahydro-2H- pyran-2- yl)ethyl] cyclobutanamine | LC-MS, m/z; 428 [M + H]+ |
| 599 | 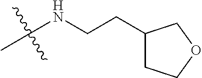 [SMILES: *NCCC1CCOC1] | 3-{3-[7-fluoro-3- (propan-2-yl)-1H- indazol-1-yl]- 1,2,4-oxadiazol-5- yl}-N-[2- (tetrahydrofuran- 3-yl)ethyl] cyclobutanamine | LC-MS, m/z; 414 [M + H]+ |
| 600 | 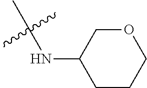 [SMILES: *NC1CCCOC1] | N-(3-{3-[7-fluoro- 3-(propan-2-yl)- 1H-indazol-1-yl]- l,2,4-oxadiazol-5- yl}cyclobutyl) tetrahydro-2H- pyran-3-amine | LC-MS, m/z; 400 [M + H]+ |
| 601 | 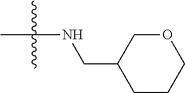 [SMILES: *NCC1CCCOC1] | 3-{3-[7-fluoro-3- (propan-2-yl)-1H- indazol-1-yl]- 1,2,4-oxadiazol-5- yl}-N-{tetrahydro- 2H-pyran-3- ylmethyl) cyclobutanamine | LC-MS, m/z; 414 [M + H]+ |
| 602 | 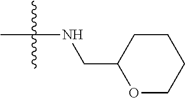 [SMILES: *NCC1CCCCO1] | 3-{3-[7-fluoro-3- (propan-2-yl)-1H- indazol-1-yl]- 1,2,4-oxadiazol-5- yl}-N-(tetrahydro- 2H-pyran-2- ylmethyl) cyclobutanamine | LC-MS, m/z; 414 [M + H]+ |
| 603 | 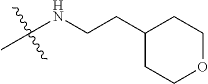 [SMILES: *NCCC1CCOCC1] | 3-{3-[7-fluoro-3- (propan-2-yl)-1H- indazol-1-yl]- 1,2,4-oxadiazol-5- yl}-N-[2- (tetrahydro-2H- pyran-4- yl)ethyl] cyclobutanamine | LC-MS, m/z; 450 [M + Na]+ |
| 604 | 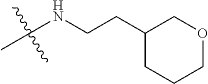 [SMILES: *NCCC1CCCOC1] | 3-{3-[7-fluoro-3- (propan-2-yl)-1H- indazol-1-yl]- 1,2,4-oxadiazol-5- yl}-N-[2- (tetrahydro-2H- pyran-3- yl)ethyl] cyclobutanamine | LC-MS, m/z; 450 [M + Na]+ |
| 605 | 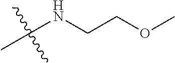 [SMILES: *NCCOC] | 3-{3-[7-fluoro-3- (propan-2-yl)-1H- indazol-1-yl]- 1,2,4-oxadiazol-5- yl}-N-(2- methoxyethyl) cyclobutanamine | LC-MS, m/z; 374 [M + H]+ |
| 606 | 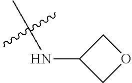 [SMILES: *NC1COC1] | N-(3-{3-[7-fluoro- 3-(propan-2-yl)- 1H-indazol-1-yl]- 1,2,4-oxadiazol-5- yl}cyclobutyl) oxetan-3-amine | LC-MS, m/z; 394 [M + Na]+ |
| 607 | 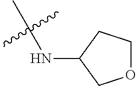 [SMILES: *NC1CCOC1] | N-(3-{3-[7-fluoro- 3-(propan-2-yl)- 1H-indazol-1-yl] - 1,2,4-oxadiazol-5- yl)cyclobutyl) tetrahydrofuran-3- amine | LC-MS, m/z; 386 [M + H]+ |
| 608 | 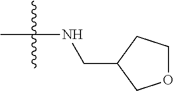 [SMILES: *NCC1CCOC1] | 3-{3-[7-fluoro-3- (propan-2-yl)-1H- indazol-1-yl]- 1,2,4-oxadiazol-5- yl}-N- (tetrahydrofuran- 3-ylmethyl) cyclobutanamine | LC-MS, m/z; 400 [M + H]+ |
| 609 | 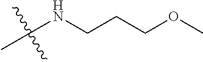 [SMILES: *NCCCOC] | 3-{3-[7-fluoro-3- (propan-2-yl)-1H- indazol-1-yl]- 1,2,4-oxadiazol-5- yl}-N-(3- methoxypropyl) cyclobutanamine | LC-MS, m/z; 388 [M + H]+ |
| 610 | 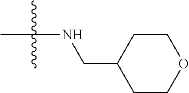 [SMILES: *NCC1CCOCC1] | 3-{3-[7-fluoro-3- (propan-2-yl)-1H- indazol-1-yl]- 1,2,4-oxadiazol-5- yl}-N-(tetrahydro- 2H-pyran-4- ylmethyl) cyclobutanamine | LC-MS, m/z; 414 [M + H]+ |

### Preparations of Examples 611 to 615

The compounds in the following table (i.e. Examples 611 to 615) were prepared in the same manner as in Example 097 except that the 3-ethyl-6-fluoro-1-[5-(piperidin-4-yl)-1,2,4-oxadiazol-3-yl]-1H-indazole trifluoroacetate and ethyl iodide were replaced with the corresponding starting compound (see, Examples 595, 596, and 610) and R₂—X, respectively.

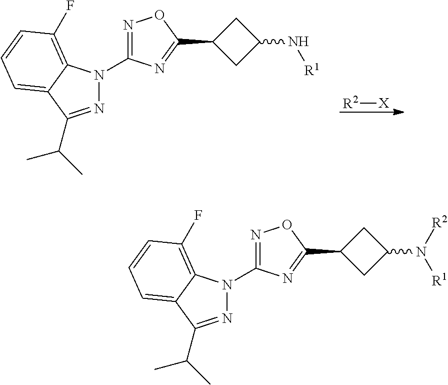

Wherein R₂—X means N-(2-chloroethyl)acetamide or 1-bromo-2-methoxyethane. Each free form of the compounds in the following table was obtained by omitting the conversion step into hydrochloride in Example 097.

TABLE 88

| Example | R¹ | R² | Compound Name | ¹H NMR/LC-MS, m/z |
|---|---|---|---|---|
| 611 | 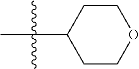 [SMILES: *C1CCOCC1] | 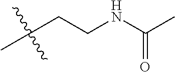 [SMILES: *CCNC(C)=O] | N-{2-[(3-{3-[7- fluoro-3-(propan-2- yl)-1H-indazol-1- yl]-1,2,4-oxadiazol- 5-yl}cyclobutyl) (tetrahydro-2H- pyran-4-yl)amino] ethyl}acetamide | LC-MS, m/z; 507 [M + Na]+ |
| 612 | 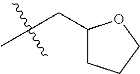 [SMILES: *CC1CCCO1] | 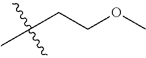 [SMILES: *CCOC] | 3-{3-[7-fluoro-3- (propan-2-yl)-1H- indazol-1-yl]-1,2,4- oxadiazol-5-yl}-N- (2-methoxyethyl)-N- (tetrahydrofuran-2- ylmethyl) cyclobutanamine | LC-MS, m/z; 458 [M + H]+ |
| 613 | 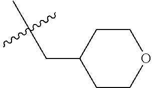 [SMILES: *CC1CCOCC1] | | 3-{3-[7-fluoro-3- (propan-2-yl)-1H- indazol-1-yl]-1,2,4- oxadiazol-5-yl}-N- (2-methoxyethyl)-N- {tetrahydro-2H- pyran-4- ylmethyl) cyclobutanamine | LC-MS, m/z; 494 [M + Na]+ |
| 614 | 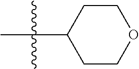 [SMILES: *C1CCOCC1] | | N-(cis-3-{3-[7- fluoro-3-(propan-2- yl)-1H-indazol-1- yl]-1,2,4-oxadiazol- 5-yl}cyclobutyl)-N- (2-methoxyethyl) tetrahydro-2H- pyran-4-amine | LC-MS, m/z; 458 [M + H]+ |
| 615 | 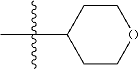 [SMILES: *C1CCOCC1] | 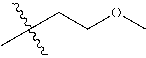 [SMILES: *CCOC] | N-(trans-3-{3-[7- fluoro-3-(propan-2- yl)-1H-indazol-1- yl]-1,2,4-oxadiazol- 5-yl}cyclobutyl)-N- (2-methoxyethyl) tetrahydro-2H- pyran-4-amine | LC-MS, m/z; 458 [M + H]+ |

### Preparations of Examples 616 to 623

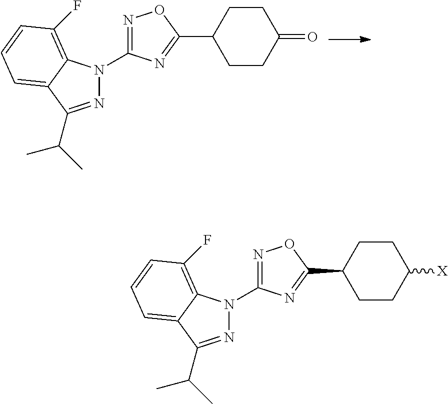

Wherein X means each structure shown in the following table.

The compounds in the following table (i.e. Examples 616 to 623) were prepared in the same manner as in Example 272 except that the 4-[3-(3-ethyl-1H-indazol-1-yl)-1,2,4-oxadiazol-5-yl]cyclohexanone and morpholine were replaced with 4-{3-[7-fluoro-3-(propan-2-yl)-1H-indazol-1-yl]-1,2,4-oxadiazol-5-yl}cyclohexanone and the corresponding amine, respectively.

TABLE 89

| Example | X | Compound Name | ¹H NMR/LC-MS, m/z |
|---|---|---|---|
| 616 | 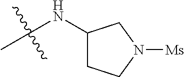 [SMILES: *NC1CCN(S(C)(=O)=O)C1] | N-(cis-4-{3-[7-fluoro-3- (propan-2-yl)-1H-indazol- 1-yl]-1,2,4-oxadiazol-5- yl}cyclohexyl)-1- (methylsulfonyl) pyrrolidin-3-amine | LC-MS, m/z; 491 [M + H]+ |
| 617 | 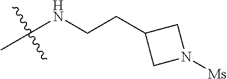 [SMILES: *NCCC1CN(S(C)(=O)=O)C1] | cis-4-{3-[7-fluoro-3- (propan-2-yl)-1H-indazol- 1-yl]-1,2,4-oxadiazol-5- yl}-N-{2- [1- (methylsulfonyl) azetidin-3-yl]ethyl} cyclohexanamine | LC-MS, m/z; 527 [M + Na]+ |
| 618 | 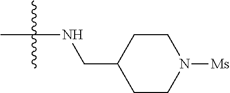 [SMILES: *NCC1CCN(S(C)(=O)=O)CC1] | cis-4-{3-[7-fluoro-3- (propan-2-yl)-1H-indazol- 1-yl]-1,2,4-oxadiazol-5- yl}-N-{[1- (methylsulfonyl) piperidin-4-yl]methyl} cyclohexanamine | LC-MS, m/z; 519 [M + H]+ |
| 619 | 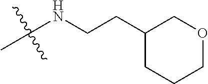 [SMILES: *NCCC1CCCOC1] | 4-{3-[7-fluoro-3-(propan- 2-yl)-1H-indazol-1-yl]- 1,2,4-oxadiazol-5-yl}-N- [2-(tetrahydro-2H-pyran- 3-yl)ethyl] cyclohexanamine | LC-MS, m/z; 456 [M + H]+ |
| 620 | 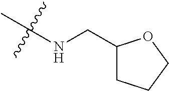 [SMILES: *NCC1CCCO1] | trans-4-{3-[7-fluoro-3- (propan-2-yl)-1H-indazol- 1-yl]-1,2,4-oxadiazol-5- yl}-N-(tetrahydrofuran-2- ylmethyl)cyclohexanamine | LC-MS, m/z; 428 [M + H]+ |
| 621 | 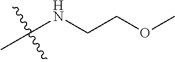 [SMILES: *NCCOC] | 4-{3-[7-fluoro-3-(propan- 2-yl)-1H-indazol-1-yl]- 1,2,4-oxadiazol-5-yl}-N- (2-methoxyethyl) cyclohexanamine | LC-MS, m/z; 402 [M + H]+ |
| 622 | 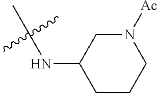 [SMILES: *NC1CCCN(C(C)=O)C1] | 1-{3-[(cis-4-{3-[7- fluoro-3-(propan-2-yl)- 1H-indazol-1-yl]-1,2,4- oxadiazol-5- yl}cyclohexyl)amino] piperidin-1-yl}ethanone | LC-MS, m/z; 469 [M + H]+ |
| 623 | 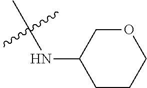 [SMILES: *NC1CCCOC1] | N-(cis-4-{3-[7-fluoro-3- (propan-2-yl)-1H-indazol- 1-yl]-1,2,4-oxadiazol-5- yl}cyclohexyl) tetrahydro-2H- pyran-3-amine | LC-MS, m/z; 428 [M + H]+ |

### Example 624

### Preparation of 1-{4-[3-(7-fluoro-3-methoxy-1H-indazol-1-yl)-1,2,4-oxadiazol-5-yl]-1,4′-bipiperidin-1′-yl}ethanone

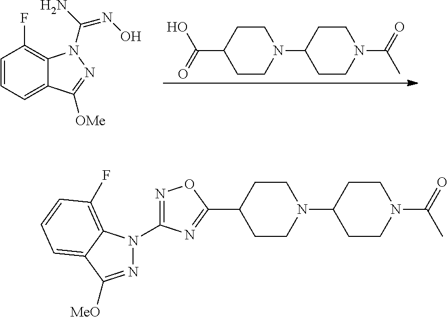 [SMILES: COc1nn(/C(N)=N\O)c2c(F)cccc12 ; CC(=O)N1CCC(N2CCC(C(=O)O)CC2)CC1 ; COc1nn(-c2noc(C3CCN(C4CCN(C(C)=O)CC4)CC3)n2)c2c(F)cccc12]

The title compound was prepared in the same manner as in Example 334 except that the 7-fluoro-N′-hydroxy-3-(propan-2-yl)-1H-indazole-1-carboximidamide and 1′-(tert-butoxycarbonyl)-1,4′-bipiperidine-4-carboxylic acid were replaced with 7-fluoro-N′-hydroxy-3-methoxy-1H-indazole-1-carboximidamide and 1′-acetyl-1,4′-bipiperidine-4-carboxylic acid, respectively, and the tetramethyl ammonium hydroxide aqueous solution was replaced with 1M tetrabutylammonium fluoride/THF.

¹H-NMR (CDCl₃) δ: 1.36-1.57 (2H, m), 1.78-2.25 (9H, m), 2.30-2.45 (2H, m), 2.48-2.63 (2H, m), 2.92-3.11 (4H, m), 3.87 (1H, d, J=13.4 Hz), 4.20 (3H, s), 4.66 (1H, d, J=13.2 Hz), 7.16-7.29 (2H, m), 7.46-7.54 (1H, m).

LC-MS, m/z; 443 [M+H]+

### Preparations of Examples 625 to 626

The compounds in the following table (i.e. Examples 625 to 626) were prepared in the same manner as in Example Example 028 except that the 3-ethyl-1-[5-(piperidin-4-yl)-1,2,4-oxadiazol-3-yl]-1H-indazole trifluoroacetate and (S)-(−)-1-tert-butoxycarbonyl-2-pyrrolidine-carbaldehyde were replaced with the corresponding starting compound and the corresponding ketone compound, respectively.

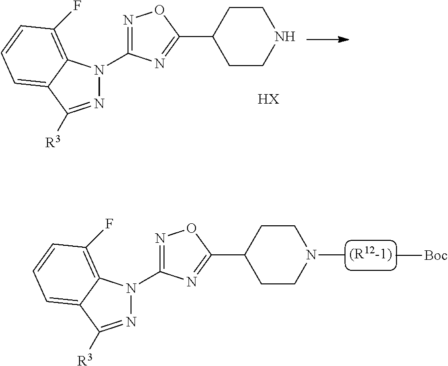

Wherein (R¹²-1) means each cyclic amino structure shown in the following table; HX means hydrochloric acid or trifluoroacetic acid; and Boc group is attached to the nitrogen atom in the cyclic amine of (R¹²-1).

TABLE 90

| Exam- | | | Compound | ¹H-NMR/ |
|---|---|---|---|---|
| ple | R³ | (R¹²-1) | Name | LC-MS, m/z |
| 625 | ⁱPr | 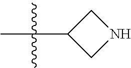 [SMILES: *C1CNC1] | tert-butyl 3-(4- {3-[7-fluoro-3- (propan-2-yl)- 1H-indazol-1- yl]-1,2,4- oxadiazol-5- yl}piperidin-1- yl)azetidine-1- carboxylate | LC-MS, m/z; 485 [M + H]+ |
| 626¹⁾ | Et | 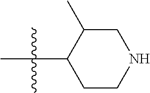 [SMILES: *C1CCNCC1C] | tert-butyl 4-[3- (3-ethyl-7- fluoro-1H- indazol-1-yl)- 1,2,4-oxadiazol- 5-yl]-3′- methyl-1,4′- bipiperidine-1′- carboxylate | LC-MS, m/z; 513 [M + H]+ |

¹⁾Titanium tetraisopropoxide was added to the reaction system.

### Preparations of Examples 627 to 628

The compounds in the following table (i.e. Examples 627 to 628) were prepared in the same manner as in Example 054 except that the tert-butyl 3-[(4-{3-[7-fluoro-3-(propan-2-yl)-1H-indazol-1-yl]-1,2,4-oxadiazol-5-yl}piperidin-1-yl)methyl]azetidine-1-carboxylate was replaced with the corresponding starting compound.

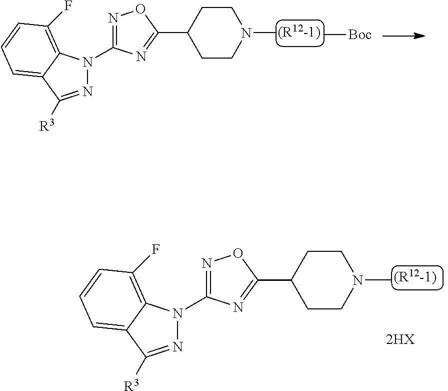

Wherein HX means trifluoroacetic acid.

TABLE 91

| Exam- | | | Compound | |
|---|---|---|---|---|
| ple | R³ | (R¹²-1) | Name | LC-MS, m/z |
| 627 | ⁱPr | 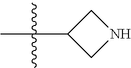 [SMILES: *C1CNC1] | 1-{5-[1- (azetidin-3- yl)piperidin- 4-yl]-1,2,4- oxadiazol-3- yl}-7-fluoro- 3-(propan-2- yl)-1H- indazole bis (trifluoro- acetate) | LC-MS, m/z; 385 [M + H]+ |
| 628 | Et | 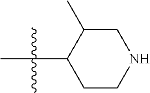 [SMILES: *C1CCNCC1C] | 4-[3-(3-ethyl- 7-fluoro-1H- indazol-1-yl)- 1,2,4-oxadiazol- 5-yl]-3′- methyl-1,4′- bipiperidine bis (trifluoro- acetate) | LC-MS, m/z; 413 [M + H]+ |

### Preparations of Examples 629 to 633

The compounds in the following table (i.e. Examples 629 to 633) were prepared in the same manner as in Example 168 except that the 3-ethyl-1-(5-{1-[2-(piperidin-4-yl)ethyl]piperidin-4-yl}-1,2,4-oxadiazol-3-yl)-1H-indazole bis(trifluoroacetate) and methyl chloroformate were replaced with the corresponding starting compound and an acid chloride (R—Cl) or acetic anhydride, respectively.

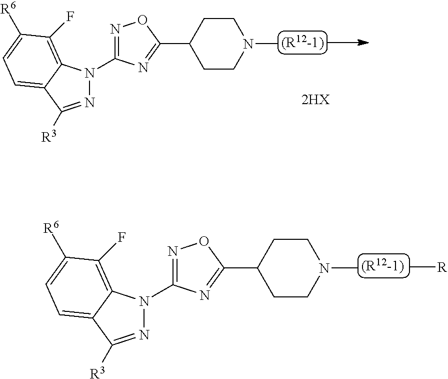

Wherein HX means hydrochloric acid or trifluoroacetic acid; (R¹²-1) means each cyclic amino structure shown in the following table; R means each structure shown in the following table; and R is attached to the nitrogen atom in the cyclic amine of (R¹²-1). Each free form of the compounds in the following table was obtained by omitting the conversion step into hydrochloride in Example 168.

TABLE 92

| Example | R³ | R⁶ | (R¹²-1) | R | Compound Name | ¹H NMR/LC-MS, m/z |
|---|---|---|---|---|---|---|
| 629 | ⁱPr | F | 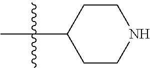 [SMILES: *C1CCNCC1] | Ms | 4-{3-[6,7- difluoro-3- (propan-2-yl)- 1H-indazol-1- yl]-1,2,4- oxadiazol-5- yl}-1′- (methylsulfonyl)- 1,4′-bipiperidine | ¹H-NMR (CDCl₃) δ: 1.48 (6H, d, J = 7.1 Hz), 1.61-1.77 (2H, m), 1.84-1.95 (2H, m), 1.95-2.10 (2H, m), 2.12-2.25 (2H, m), 2.34-2.50 (3H, m), 2.66-2.77 (2H, m), 2.79 (3H, s), 2.91-3.10 (3H, m), 3.37-3.52 (1H, m), 3.78-3.90 (2H, m), 7.10-7.20 (1H, m), 7.46-7.53 (1H, m). LC-MS, m/z; 509 [M + H]+ |
| 630 | Et | F | | | 4-[3-(3-ethyl- 6,7-difluoro- 1H-indazol-1- yl)-1,2,4- oxadiazol-5- yl]-1′- (methylsulfonyl)- 1,4′-bipiperidine | ¹H-NMR (CDCl₃) δ: 1.43 (3H, t, J = 7.6 Hz), 1.61-1.76 (2H, m), 1.85-1.95 (2H, m), 1.97-2.10 (2H, m), 2.14-2.24 (2H, m), 2.35-2.50 (3H, m), 2.66-2.77 (2H, m), 2.79 (3H, s), 2.93-3.10 (5H, m), 3.80-3.90 (2H, m), 7.13-7.22 (1H, m), 7.42-7.48 (1H, m). LC-MS, m/z; 495 [M + H]+ |
| 631 | Et | H | 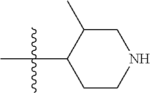 [SMILES: *C1CCNCC1C] | Ac | 1-{4-[3-(3- ethyl-7- fluoro-1H- indazol-1-yl)- 1,2,4- oxadiazol-5- yl]-3′-methyl- 1,4′-bipiperidin- 1′-yl}ethanone | ¹H-NMR (CDCl₃) δ: 0.83-0.98 (3H, m), 1.23-1.51 (4H, m), 1.71-2.29 (12H, m), 2.38-2.63 (1H, m), 2.88-3.22 (6H, m), 3.60-3.89 (1H, m), 4.49-4.78 (1H, m), 7.16-7.32 (2H, m), 7.45-7.59 (1H, m). LC-MS, m/z; 455 [M + H]+ |
| 632 | | | | 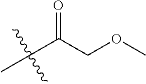 [SMILES: *C(=O)COC] | 1-{4-[3-(3- ethyl-7- fluoro-1H- indazol-1-yl)- 1,2,4- oxadiazol-5- yl]-3′-methyl- 1,4′-bipiperidin- 1′-yl}-2- methoxy- ethanone | ¹H-NMR (CDCl₃) δ: 0.87-0.98 (3H, m), 1.20-1.62 (4H, m), 1.76-1.87 (2H, m), 1.95-2.29 (8H, m), 2.43-2.71 (1H, m), 2.86-3.16 (6H, m), 3.42 (3H, d, J = 2.9 Hz), 3.66-3.99 (1H, m), 4.03-4.24 (2H, m), 4.47-4.73 (1H, m), 7.20-7.30 (2H, m), 7.48-7.58 (1H, m) LC-MS, m/z; 485 [M + H]+ |
| 633 | | | | Ms | 4-[3-(3-ethyl- 7-fluoro-1H- indazol-1-yl)- 1,2,4- oxadiazol-5- yl]-3′-methyl - 1′- (methyl- sulfonyl)-1,4′- bipiperidine | ¹H-NMR (CDCl₃) δ: 1.04 (3H, d, J = 6.8 Hz), 1.42 (3H, t, J = 7.6 Hz), 1.60-1.88 (2H, m), 1.94-2.27 (9H, m), 2.53-2.67 (1H, m), 2.75 (3H, s), 2.97-3.13 (5H, m), 3.56-3.66 (1H, m), 3.78-3.87 (1H, m), 7.16-7.29 (2H, m), 7.47-7.54 (1H, m). LC-MS, m/z; 491 [M + H]+ |

### Preparations of Examples 634 to 636

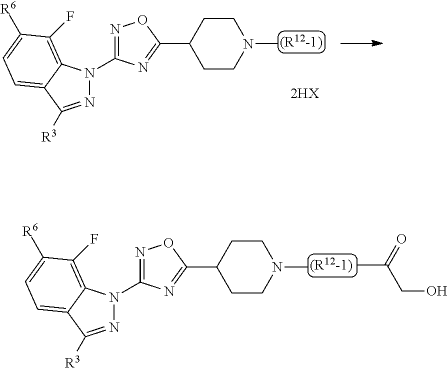

Wherein HX means hydrochloric acid or trifluoroacetic acid; and (R¹²-1) means each cyclic amino structure shown in the following table; and the hydroxyacetyl group is attached to the nitrogen atom in the cyclic amine of (R¹²-1).

The compounds in the following table (i.e. Examples 634 to 636) were prepared in the same manner as in Example 242 except that the 4-{3-[7-fluoro-3-(propan-2-yl)-1H-indazol-1-yl]-1,2,4-oxadiazol-5-yl}-1,4′-bipiperidine dihydrochloride was replaced with the corresponding starting compound, or the 2 N sodium hydroxide aqueous solution was replaced with methyl amine/methanol.

TABLE 93

| Exam- | | | | Compound | ¹H-NMR/ |
|---|---|---|---|---|---|
| ple | R³ | R⁶ | (R¹²-1) | Name | LC-MS, m/z |
| 634 | ⁱPr | H | 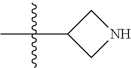 [SMILES: *C1CNC1] | 1-[3-(4-{3- [7-fluoro-3- (propan-2- yl)-1H- indazol-1- yl]-1,2,4- oxadiazol- 5-yl} piperidin- 1-yl) azetidin- 1-yl]-2- hydroxy- ethanone | ¹H-NMR (CDCl₃) δ: 1.50 (6H, d, J = 7.1 Hz), 2.01- 2.29 (6H, m), 2.77-2.93 (2H, m), 3.04-3.17 (2H, m), 3.24- 3.34 (1H, m), 3.41-3.55 (1H, m), 3.91-4.02 (4H, m), 4.04- 4.19 (2H, m), 7.18-7.28 (2H, m), 7.56-7.62 (1H, m). LC-MS, m/z; 443 [M + H]+ |
| 635 | Et | F | 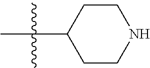 [SMILES: *C1CCNCC1] | 1-{4-[3-(3- ethyl-6,7- difluoro-1H- indazol-1- yl)-1,2,4- oxadiazol- 5-yl]-1,4′- bipiperidin- 1′-yl}-2- hydroxy- ethanone | ¹H-NMR (CDCl₃) δ: 1.40- 1.58 (2H, m), 1.43 (3H, t, J = 7.6 Hz), 1.83-1.95 (2H, m), 1.95-2.10 (2H, m), 2.14- 2.25 (2H, m), 2.34-2.47 (2H, m), 2.53-2.65 (1H, m), 2.68- 2.80 (1H, m), 2.90-3.10 (6H, m), 3.50-3.70 (2H, m), 4.16 (2H, s), 4.55- 4.68 (1H, m), 7.12-7.22 (1H, m), 7.42-7.49 (1H, m). LC-MS, m/z; 475 [M + H]+ |
| 636 | Et | H | 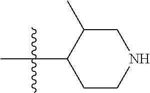 [SMILES: *C1CCNCC1C] | 1-{4-[3-(3- ethyl-7- fluoro-1H- indazol-1- yl)-1,2,4- oxadiazol- 5-yl]-3′- methyl-1,4′- bipiperidin- 1′-yl}-2- hydroxy- ethnone | ¹H-NMR (CDCl₃) δ: 0.86- 0.94 (3H, m), 1.36-1.57 (4H, m), 1.73-1.89 (2H, m), 1.93- 2.30 (7H, m), 2.52-2.82 (1H, m), 2.87-3.16 (6H, m), 3.27- 3.59 (1H, m), 3.66 (1H, br s), 4.01- 4.26 (2H, m), 4.40-4.72 (1H, m), 7.17- 7.29 (2H, m), 7.49-7.56 (1H, m). LC-MS, m/z; 471 [M + H]+ |

### Example 637

### Preparation of 1-(4-{3-[7-fluoro-3-(propan-2-yl)-1H-indazol-1-yl]-1,2,4-oxadiazol-5-yl}-1,4′-bipiperidin-1′-yl)-2-hydroxyethanone (Form A and Form B)

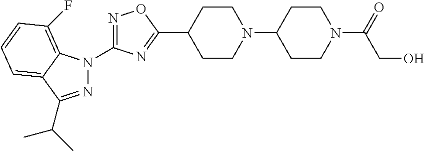 [SMILES: CC(C)c1nn(-c2noc(C3CCN(C4CCN(C(=O)CO)CC4)CC3)n2)c2c(F)cccc12]

Form A: 1-(4-{3-[7-Fluoro-3-(propan-2-yl)-1H-indazol-1-yl]-1,2,4-oxadiazol-5-yl}-1,4′-bipiperidin-1′-yl)-2-hydroxyethanone prepared in Example 242 (33 g) was suspended in 2-propanol (45 mL), and then the suspension was stirred at 85° C. to dissolve the compound. The solution was gradually cooled to room temperature, 2-propanol (9 mL) was added thereto, and then the reaction mixture was stored in a refrigerator for four days. The precipitated crystal was collected on a filter, washed with cold 2-propanol, and then dried in vacuo at 80° C. to give the title compound (30.8 g) as a white crystal which is characterized by the following X-ray diffraction peaks.

### XRD; 2θ=5.22, 10.42, 10.71, 11.16, 11.91, 12.71, 13.98, 14.61, 15.36, 15.64, 15.92, 16.83, 17.47, 18.27, 18.75, 19.46, 20.16, 20.56, 21.43, 21.74

Form B: 1-(4-{3-[7-fluoro-3-(propan-2-yl)-1H-indazol-1-yl]-1,2,4-oxadiazol-5-yl}-1,4′-bipiperidin-1′-yl)-2-hydroxyethanone prepared in Example 242 (116 g) was suspended in 2-propanol (350 mL), and then the suspension was stirred at 85° C. to dissolve the compound. The solution was gradually cooled to room temperature, and after confirming that a crystal was precipitated, the mixture was stirred at −10° C. for two hours. The precipitated crystal was collected on a filter, washed with cold 2-propanol (350 mL), and then dried in vacuo at 60° C. to give the title compound (106 g) as a white crystal which is characterized by the following X-ray diffraction peaks.

### XRD; 2θ=8.00, 8.63, 9.87, 12.50, 13.58, 14.73, 15.07, 15.99, 16.39, 16.73, 17.73, 18.42, 19.38, 20.78, 21.31, 22.08, 22.48, 23.28, 23.63, 23.98

The X-ray diffraction (XRD) measurement was carried out using X-ray diffraction system X′ pert MPD (PANAlytical) under the following condition:

Anode material: Copper,

K-Alpha1: 1.54 Å,

Tension: 45 kV,

Current: 40 mA,

Start angle (2θ): 4°,

End angle (2θ): 40°,

Step size (2θ): 0.017°, and

Time per step: 100 s.

In detail, the measurement was carried out under the above condition, using invisible Si plate as a sample assay plate which was coated with about 5 mg of the sample. The X-ray diffraction measurement of the samples mentioned below was also carried out as the same.

### Example 638

### Preparation of 1-(4-{3-[7-fluoro-3-(propan-2-yl)-1H-indazol-1-yl]-1,2,4-oxadiazol-5-yl}-1,4′-bipiperidin-1′-yl)-2-hydroxyethanone hydrochloride

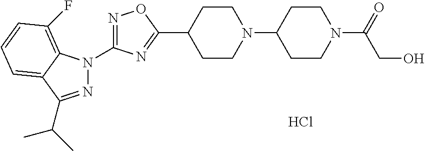 [SMILES: CC(C)c1nn(-c2noc(C3CCN(C4CCN(C(=O)CO)CC4)CC3)n2)c2c(F)cccc12 ; Cl]

1-(4-{3-[7-Fluoro-3-(propan-2-yl)-1H-indazol-1-yl]-1,2,4-oxadiazol-5-yl}-1,4′-bipiperidin-1′-yl)-2-hydroxyethanone prepared in Example 242 (2.44 g) was suspended in 2-propanol (36 ml) and ethanol (27 mL) and heated to 85° C. to be dissolved. Then, 1N HCl/diethyl ether (4.93 mL) was added thereto, and the mixture was stirred at room temperature overnight. The precipitated crystal was collected on a filter and suspended in 2-propanol (7 mL). The suspension was heated at 85° C. to dissolve the crystal, and then the solution was gradually cooled to room temperature for recrystallization. The resulting crystal was collected on a filter to give the title compound (2.09 g) as a white crystal which is characterized by the following X-ray diffraction peaks.

### XRD; 2θ=5.28, 9.75, 10.55, 11.91, 12.47, 13.39, 14.63, 15.31, 15.69, 16.07, 16.37, 17.19, 17.82, 18.25, 18.63, 19.20, 19.51, 19.88, 20.69, 21.18

### Examples 639 to 645

The following table shows the prepared compounds and X-ray diffraction peaks thereof.

TABLE 94

| Example | Compound Name | 2θ |
|---|---|---|
| 639¹⁾ | 1-{5-[1-(3- | 7.97, 9.13, 10.55, 10.91, |
| | methoxypropyl)piperidin-4- | 11.94, 12.32, 12.91, 13.59, |
| | yl]-1,2,4-oxadiazol-3-yl}-3- | 14.90, 15.51, 15.98, 16.93, |
| | (propan-2-yl)-1H-indazole | 17.62, 17.86, 18.17, 18.70, |
| | hydrochloride | 19.67, 20.15, 21.23, 21.86 |
| | (prepared in Example 85) | |
| 640 | 3-{4-[3-(3-ethyl-1H-indazol- | 6.60, 10.99, 11.25, 12.38, |
| | 1-yl)-1,2,4-oxadiazol-5- | 14.42, 15.12, 15.60, 16.34, |
| | yl]piperidin-1-yl}propan-1-ol | 16.84, 17.81, 19.78, 20.45, |
| | (prepared in Example 261) | 21.29, 21.88, 22.79, 23.19, |
| | | 23.76, 24.64, 26.14 |
| 641 | 1-{4-[3-(3-ethyl-6-fluoro-1H | 5.13, 9.93, 13.34, 13.68, |
| | indazol-1-yl)-1,2,4- | 15.50, 17.08, 17.62, 18.45, |
| | oxadiazol-5-yl]-1,4′- | 19.80, 19.96, 20.66, 21.14, |
| | bipiperidin-1′-yl}ethanone | 21.92, 22.29, 22.50, 23.15, |
| | hydrochloride | 24.28, 24.66, 26.25, 26.41 |
| | (prepared in Example 228) | |
| 642 | 1-{4-[3-(3-ethyl-7-fluoro-1H- | 5.34, 8.57, 9.71, 10.58, |
| | indazol-1-yl)-1,2,4- | 11.36, 13.22, 13.66, 14.40, |
| | oxadiazol-5-yl]-1,4′- | 15.00, 15.58, 15.86, 16.30, |
| | bipiperidin-1′-yl}ethanone | 16.52, 16.96, 17.22, 17.84, |
| | hydrochloride | 18.20, 19.46, 20.28, 21.18, |
| | (prepared in Example 229) | 21.62 |
| 643 | methyl 4-[3-(3-ethyl-6- | 5.90, 6.62, 9.93, 10.99, |
| | fluoro-1H-indazol-1-yl)- | 11.80, 12.06, 13.24, 15.18, |
| | 1,2,4-oxadiazol-5-yl]-1,4′- | 16.76, 17.42, 18.11, 19.33, |
| | bipiperidin-1′-carboxylate | 20.16, 20.83, 21.14, 21.45, |
| | (prepared in Example 230) | 22.06, 22.36, 22.61, 23.68 |
| 644 | 1-(4-{3-[7-fluoro-3-(propan- | 5.15, 9.54, 10.24, 13.02, |
| | 2-yl)-1H-indazol-1-yl]-1,2,4- | 13.81, 15.11, 15.60, 16.10, |
| | oxadiazol-5-yl}-1,4′- | 16.74, 17.35, 18.32, 18.68, |
| | bipiperidin-1′-yl)ethanone | 19.10, 19.65, 19.90, 20.49, |
| | hydrochloride | 21.08, 22.04, 22.52, 23.35 |
| | (prepared in Example 232) | |
| 645²⁾ | 1-(4-{3-[7-fluoro-3-(propan- | 5.11, 10.20, 11.24, 11.78, |
| | 2-yl)-1H-indazol-1-yl]-1,2,4- | 13.66, 14.10, 14.94, 15.30, |
| | oxadiazol-5-yl}-1,4′- | 15.62, 15.94, 16.32, 17.07, |
| | bipiperidin-1′-yl)ethanone | 17.38, 18.07, 18.74, 19.56, |
| | | 20.34, 20.65, 21.27, 21.59, |
| | | 22.65 |

¹⁾prepared by treating the compound prepared in Example 85 with 1N HCl/diethyl ether.
²⁾prepared in the same manner as in Example 637 from the free base compound prepared in Example 232 provided that the conversion step into hydrochloride was omitted.

### Examples 646 to 650

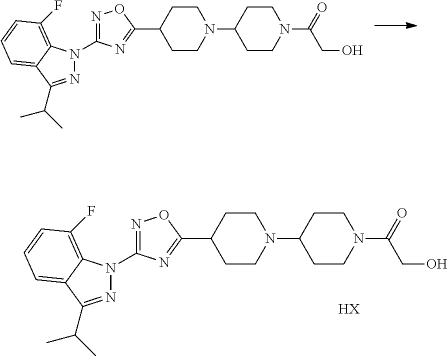

Wherein HX means the acid shown in the following table.

The salt compounds in the following table (i.e. Examples 646 to 650) were prepared in the same manner as in Example 638, provided using the corresponding acid (HX) instead of HCl, solvent and procedure shown in the following table. The equivalent of HX shown in the following table means an equivalent thereof for the free base compound which was used in the preparation process.

XRPD analyses were performed using a Rigaku MiniFlex II Desktop X-Ray diffractometer using Cu radiation. The tube voltage and amperage were set to 30 kV and 15 mA, respectively. The scattering slit was fixed at 1.25° and the receiving slit was fixed at 0.3 mm. Diffracted radiation was detected by a NaI scintillation detector. A 6-26 continuous scan at 1.0°/min with a step size of 0.02-0.05° from 3 to 45° 26 was used. Data were collected and analyzed using Jade 8.5.4. Each sample was prepared for analysis by placing it in a low background, round, 0.1 mm indent sample holder

TABLE 95

| Example | Salt (HX) | Solvent(s) | Procedure | 2θ |
|---|---|---|---|---|
| 646 | Besylate | Ethyl | The freebase | 4.30, 8.62, |
| | (1.05 | acetate | (22.3 mg), | 8.90, 9.92, |
| | eq.) | | acid, and | 11.52, 12.70, |
| | | | solvent were | 13.74, 16.00, |
| | | | heated to | 17.15, 17.92, |
| | | | 70° C., held | 22.28, 23.86, |
| | | | for 1 hour, | 25.26, 26.88 |
| | | | and cooled | |
| | | | to room temperature | |
| 647 | Besylate | Ethyl | Acid solution in IPA | 4.32, 8.96, |
| | (1.35 | acetate/ | was added to | 9.96, 11.56, |
| | eq.) | isopropanol | freebase (387.8 mg) | 12.76, 13.76, |
| | | | solution in | 14.14, 14.98, |
| | | | IPA at 70° C. then | 15.76, 16.42, |
| | | | cooled to 0° C. over | 17.20, 17.96, |
| | | | 3 hours and held | 18.46, 19.86, |
| | | | overnight (product | 21.30, 21.74, |
| | | | oiled out). | 22.30, 22.80, |
| | | | System was | 23.12, 23.86 |
| | | | reheated to 60° C., | |
| | | | ethyl acetate added, | |
| | | | then cooled to 0° C. | |
| | | | over 2 hours. | |
| | | | 425.8 mg | |
| | | | of salt was obtained. | |
| | | | (0.8 mol counter | |
| | | | acid: 1 mol free | |
| | | | base) | |
| 648 | Besylate | 28 wt % | Acid solution | 4.44, 8.92, |
| | (0.97 | methanol in | in ethyl acetate | 9.14, 9.96, |
| | eq.) | ethyl | was added to | 11.76, 13.34, |
| | | acetate | freebase (350.9 mg) | 14.16, 16.10, |
| | | | solution in ethyl | 16.39, 17.88, |
| | | | acetate at | 18.44, 19.42, |
| | | | 60° C. then | 20.56, 21.04, |
| | | | cooled to 0° C. over | 21.42, 22.38, |
| | | | 80 minutes. | 22.90, 23.70, |
| | | | Heat this | 25.34, 26.20, |
| | | | besylate salt (363.6 | 27.46, 28.50, |
| | | | mg) in ethyl acetate | 29.32, 30.32, |
| | | | to 67° C. and | 31.30, 32.60, |
| | | | add enough | 33.60, 34.51, |
| | | | methanol to | 35.46 |
| | | | dissolve. | |
| | | | Add acid solution in | |
| | | | ethyl acetate | |
| | | | and cool | |
| | | | to R.T. over 1 hour. | |
| | | | 241.9 mg | |
| | | | of salt was | |
| | | | obtained. (0.9 mol | |
| | | | counter acid: 1 mol | |
| | | | free base) | |
| 649 | L- | 10 vol % | Acid solution in | 7.18, 10.84, |
| | tartrate | methanol in | methanol/ | 11.52, 12.96, |
| | (1.09 | ethyl | ethyl acetate | 14.36, 15.42, |
| | eq.) | acetate | was added | 16.12, 17.26, |
| | | | to freebase | 17.62, 18.06, |
| | | | (387.5 mg) | 18.96, 19.59, |
| | | | solution in | 21.04, 21.88, |
| | | | methanol/ | 24.52, 25.04, |
| | | | ethyl acetate | 26.16, 27.32, |
| | | | at 70° C. | 28.78, 29.71, |
| | | | then cooled to | 30.56 |
| | | | 20° C. over | |
| | | | 60 minutes. | |
| | | | 483.5 mg | |
| | | | of salt was | |
| | | | obtained. (1 mol | |
| | | | counter acid: 1 mol | |
| | | | free base) | |
| 650 | Fumarate | 15 vol % | Salt solution in | 7.14, 7.68, |
| | (1.07 | methanol in | methanol/ | 9.90, 12.74, |
| | eq.) | ethyl | ethyl acetate | 13.61, 14.36, |
| | | acetate | was added | 14.65, 15.72, |
| | | | to freebase | 17.36, 17.86, |
| | | | (351.3 mg) | 19.74, 21.18, |
| | | | solution in | 21.62, 23.20, |
| | | | methanol/ | 24.24, 25.08, |
| | | | ethyl acetate | 26.20, 27.50, |
| | | | at 60° C. then | 28.90, 29.30, |
| | | | cooled to | 31.18 |
| | | | 0° C. over | |
| | | | 60 minutes. | |
| | | | 4325.0 mg of | |
| | | | salt was | |
| | | | obtained. (1 mol | |
| | | | counter acid: 1 mol | |
| | | | free base) | |

### (Pharmacological Test Result)

Hereinafter, some methods and results of the pharmacological tests for the representative compounds of the present invention are shown, but the present invention should not be construed as limited to these pharmacological tests.

### Test Example 1

### Serotonin 4 (5-HT₄) Receptor Binding Assay

The 5-HT₄ receptor binding assay and preparations of receptor membrane preparations herein were carried out according to a method of Grossman et al. [see, British J. Pharmacol., (1993) 109, 618].

Slc-Hartley guinea pigs (body weight 300 g to 400 g) were decapitated to remove brain rapidly, and striatum was isolated which was cryopreserved at −80° C. until use. To the obtained tissues were added fifteen fold of Hepes buffer (50 mM, pH 7.4, 4° C.), and the mixture was homogenized by Teflon (trademark) homogenizer and centrifuged at 48,000×g (4° C.) for 15 minutes. To the resulting precipitate was added Hepes buffer (1 ml) to 30 mg by wet weight of tissues, and the mixture was suspended to give receptor membrane preparations.

To an assay tube were added 0.1 nM [³H]-GR113808 {chemical name: [1-[2-(methylsulfonylamino)ethyl]-4-piperidinyl]methyl 1-methylindole-3-carboxylate}, receptor membrane preparations, and Hepes buffer (50 mM, pH 7.4, 4° C., 1 ml) containing test compounds or 30 μM serotonin; and the mixture was incubated at 37° C. for 30 minutes. On quenching the reaction, the mixture was rapidly filtered on whatman GF/B filter, which was presoaked in 0.1% polyethyleneimine for 1 hour, by using Brandel cell Harvester, and washed with ice-cooled 50 mM Tris-HCl (pH 7.7, 4 ml) three times. To the filter after filtration was added a liquid scintillator (Ecoscint), and then a radioactivity was determined by a liquid scintillation counter.

50% Inhibition concentrations (IC₅₀) were determined from inhibition rates of test compounds to specific bindings which were obtained by subtracting nonspecific bindings from total binding amounts of [³H]-GR113808.

Table 96 shows results of serotonin 4 (5-HT₄) receptor binding assay. In the following table, the compounds used in the test are shown in numbers which correspond to the Example numbers above where the preparations of the compounds are described. Each IC₅₀ shows the mean value of each group.

TABLE 96
Guinea pig 5-HT₄ receptor binding assay

| Example Number | IC₅₀ (nM) |
|---|---|
| 85 | 31.1 |
| 87 | 21.0 |
| 88 | 33.5 |
| 92 | <20 |
| 95 | <20 |
| 98 | 32.1 |
| 101 | <20 |
| 102 | <20 |
| 103 | <20 |
| 112 | 26.4 |
| 114 | 26.2 |
| 115 | 40.2 |
| 117 | 25.9 |
| 131 | <20 |
| 132 | <20 |
| 134 | 24.2 |
| 137 | <20 |
| 138 | <20 |
| 143 | <20 |
| 171 | 31.9 |
| 178 | <20 |
| 182 | <20 |
| 195 | <20 |
| 224 | <20 |
| 228 | 28.7 |
| 229 | <20 |
| 230 | 25.1 |
| 232 | <20 |
| 242 | <20 |
| 257 | <20 |
| 261 | <20 |
| 278 | <20 |

### Test Example 2

### Serotonin 4 (5-HT₄) Receptor Agonist Activity Assay

The cAMP measurement test used herein was carried out by using CISBIO HTRF (trademark) cAMP Hirange kit according to the manufacturer's instructions attached therewith.

CHO cells expressing human 5-HT_{4b} receptor were incubated in Medium 1 [DMEM/1% NEAA, 1% penicillin/streptomycin (P/S), 0.2 mg/mL GENETICIN (G418), 10% FBS] at 37° C. under 5% CO₂ condition. Then, the cells were put into Medium 2 (DMEM/10000 cut FBS, G-418, P/S, NEAA) for 1 to 2 hours, and collected by treating with trypsin containing EDTA. The collected cells were suspended in Assay Buffer 1 [100 mM Hanks/HEPES buffer (pH 7.4)], the suspended cells were mixed with the testing compound on 384-well plates, and the cells were incubated at 31° C. for 15 minutes. To the cells were added cAMP-cryptate solution and cAMP-d₂ solution, and they were incubated at room temperature for 1 hour. Then, time-resolved fluorescence was measured by En Vision (excitation wavelength: 330 nm, fluorescence wavelength: 620/665 nm). An intrinsic activity of the compound [IA (%)] and a concentration showing 50% of IA [EC₅₀ (nM)] were calculated from the obtained results. In particular, the intrinsic activity (IA) was calculated on the basis of the maximal activity of 5-HT (measured from 10⁻¹¹ M to 10⁻⁷ M) defined as 100%.

Table 97 shows results of serotonin 4 (5-HT₄) receptor agonist activity assay. In the following table, the compounds used in the test are shown in numbers which correspond to the Example numbers above where the preparations of the compounds are described. Each IA and EC₅₀ shows the mean value of each group.

TABLE 97

| Example | | |
|---|---|---|
| Number | IA (%) | EC₅₀ (nM) |
| 85 | 125 | 8.3 |
| 87 | 80 | 9.4 |
| 88 | 37 | 16.4 |
| 92 | 92 | 6.5 |
| 95 | 43 | 3.7 |
| 98 | 110 | 15.5 |
| 101 | 70 | 8.9 |
| 102 | 26 | 8.0 |
| 103 | 59 | 11.0 |
| 112 | 73 | 24.9 |
| 114 | 68 | 53.0 |
| 115 | 58 | 153 |
| 117 | 83 | 29.5 |
| 131 | 77 | 9.3 |
| 132 | 29 | 14.7 |
| 134 | 81 | 12.1 |
| 137 | 66 | 8.6 |
| 138 | 67 | 22.4 |
| 143 | 81 | 6.0 |
| 171 | 51 | 30.8 |
| 178 | 38 | 10.9 |
| 182 | 38 | 4.0 |
| 195 | 49 | 31.4 |
| 228 | 66 | 8.2 |
| 229 | 55 | 2.9 |
| 230 | 65 | 17.9 |
| 232 | 95 | 2.6 |
| 242 | 88 | 2.2 |
| 257 | 48 | 60.7 |
| 261 | 84 | 6.2 |

### Test Example 3

### Serotonin 4 (5-HT₄) Receptor Binding Assay

The guinea pig 5-HT₄ receptor binding assay and preparations of receptor membrane preparations herein were carried out according to a method of Grossman et al. [see, British J. Pharmacol., (1993) 109, 618].

Slc-Hartley guinea pigs (body weight 300 g to 400 g) were decapitated to remove brain rapidly, and striatum was isolated which was cryopreserved at −80° C. until use. To the obtained tissues were added fifteen fold of Hepes buffer (50 mM, pH 7.4, 4° C.), and the mixture was homogenized by Teflon (trademark) homogenizer and centrifuged at 48,000×g (4° C.) for 15 minutes. To the resulting precipitate was added Hepes buffer (1 ml) to 30 mg by wet weight of tissues, and the mixture was suspended to give receptor membrane preparations.

To an assay tube were added 0.1 nM [³H]-GR113808 {chemical name: [1-[2-(methylsulfonylamino) ethyl]-4-piperidinyl]methyl 1-methylindole-3-carboxylate}, receptor membrane preparations, and Hepes buffer (50 mM, pH 7.4, 4° C., 1 ml) containing test compounds or 30 μM serotonin; and the mixture was incubated at 37° C. for 30 minutes. On quenching the reaction, the mixture was rapidly filtered on whatman GF/B filter, which was presoaked in 0.1% polyethyleneimine for 1 hour, by using Brandel cell Harvester, and washed with ice-cooled 50 mM Tris-HCl (pH 7.7, 4 ml) three times. To the filter after filtration was added a liquid scintillator (Ecoscint), and then a radioactivity was determined by a liquid scintillation counter.

50% Inhibition concentrations (IC₅₀) were determined from inhibition rates of test compounds to specific bindings which were obtained by subtracting nonspecific bindings from total binding amounts of [³H]-GR113808.

Human 5-HT₄ receptor membrane preparations were prepared from CHO-K¹ cells which stably express 5-HT4b receptors, and human 5-HT₄ receptor binding assay was carried out in a similar manner as in the guinea pig 5-HT₄ receptor binding assay. The following tables show results of serotonin 4 (5-HT₄) receptor binding assay. In the following tables, the compounds used in the test are shown in numbers which correspond to the Example numbers above where the preparations of the compounds are described. Each IC₅₀ shows the mean value of each group.

TABLE 98
Guinea pig 5-HT₄ receptor binding assay

| Ex. | IC₅₀ |
|---|---|
| No. | (nM) |
| 201 | <20 |
| 202 | <20 |
| 203 | <20 |
| 209 | <20 |
| 210 | <20 |
| 216 | <20 |
| 219 | <20 |
| 220 | <20 |
| 221 | <20 |
| 223 | <20 |
| 224 | <20 |
| 233 | <20 |
| 237 | <20 |
| 241 | <20 |
| 244 | <20 |
| 246 | <20 |
| 247 | <20 |
| 250 | <20 |
| 394 | <20 |
| 395 | 26.9 |
| 396 | <20 |
| 397 | <20 |
| 398 | <20 |
| 402 | <20 |
| 403 | 36.1 |
| 404 | 100 |
| 405 | 65.4 |
| 406 | 68.7 |
| 407 | 101 |
| 408 | 29.5 |
| 409 | 383 |
| 410 | 323 |
| 412 | 140 |
| 413 | <20 |
| 414 | 86.9 |
| 415 | <20 |
| 417 | 36 |
| 420 | 73.9 |
| 421 | 129 |
| 422 | 55 |
| 427 | 339 |
| 428 | 248 |
| 431 | 130 |
| 433 | 65 |
| 434 | 20.8 |
| 435 | 44.7 |
| 436 | 179 |
| 437 | 113 |
| 440 | 104 |
| 441 | 78.2 |
| 442 | <20 |
| 443 | 18.1 |
| 445 | <20 |
| 446 | <20 |
| 447 | <20 |
| 448 | <20 |
| 449 | <20 |
| 450 | <20 |
| 451 | <20 |
| 452 | <20 |
| 453 | 28.1 |
| 454 | <20 |
| 455 | <20 |
| 456 | <20 |
| 457 | 85.3 |
| 458 | 66 |
| 459 | <20 |
| 460 | <20 |
| 461 | <20 |
| 462 | 109 |
| 463 | <20 |
| 464 | <20 |
| 465 | <20 |
| 466 | <20 |
| 467 | 81.1 |
| 468 | <20 |
| 469 | 20.4 |
| 470 | 30 |
| 471 | 173 |
| 472 | 29.7 |
| 473 | 23.8 |
| 474 | 20.2 |
| 475 | 67.9 |
| 476 | <20 |
| 477 | <20 |
| 478 | <20 |
| 479 | 94.6 |
| 480 | 27.5 |
| 481 | <20 |
| 482 | 23.6 |
| 483 | 35.7 |
| 484 | 28.1 |
| 485 | 21.2 |
| 486 | <20 |
| 495 | <20 |
| 507 | <20 |
| 519 | 27.1 |
| 520 | <20 |
| 521 | 93.7 |
| 522 | 222 |
| 529 | <20 |
| 530 | <20 |
| 532 | <20 |
| 533 | <20 |
| 551 | 81.9 |
| 552 | 179 |
| 553 | 18.7 |
| 554 | 190 |
| 555 | 24.9 |
| 557 | 127 |
| 562 | 478 |
| 563 | 222 |
| 564 | 653 |

TABLE 99
Human 5-HT₄ receptor binding assay

| Ex. | IC₅₀ |
|---|---|
| No. | (nM) |
| 232 | <20 |
| 242 | <20 |
| 399 | 20.2 |
| 400 | 225 |
| 401 | 296 |
| 416 | 27.4 |
| 418 | 442 |
| 419 | 63.8 |
| 423 | 136 |
| 424 | 55 |
| 444 | 96.8 |
| 487 | <20 |
| 488 | <20 |
| 489 | <20 |
| 490 | <20 |
| 491 | <20 |
| 492 | <20 |
| 493 | <20 |
| 494 | <20 |
| 496 | <20 |
| 497 | 76.1 |
| 498 | <20 |
| 499 | 48.6 |
| 500 | <20 |
| 501 | <20 |
| 502 | <20 |
| 503 | <20 |
| 504 | <20 |
| 505 | <20 |
| 506 | <20 |
| 508 | <20 |
| 509 | 27.4 |
| 510 | <20 |
| 511 | <20 |
| 512 | <20 |
| 513 | <20 |
| 514 | <20 |
| 515 | <20 |
| 516 | <20 |
| 517 | <20 |
| 518 | <20 |
| 523 | <20 |
| 524 | <20 |
| 525 | <20 |
| 526 | 25.2 |
| 527 | <20 |
| 528 | <20 |
| 531 | <20 |
| 534 | 42.4 |
| 535 | 29.7 |
| 536 | <20 |
| 537 | <20 |
| 538 | <20 |
| 539 | <20 |
| 540 | 78.1 |
| 541 | 35.8 |
| 542 | <20 |
| 543 | <20 |
| 544 | <20 |
| 545 | <20 |
| 546 | <20 |
| 547 | <20 |
| 548 | <20 |
| 549 | <20 |
| 550 | <20 |
| 556 | 52.1 |
| 558 | <20 |
| 559 | <20 |
| 561 | 33.3 |
| 566 | <20 |
| 582 | <20 |
| 583 | <20 |
| 585 | <20 |
| 586 | <20 |
| 590 | 12.6 |
| 591 | <20 |
| 592 | <20 |
| 593 | <20 |
| 594 | <20 |
| 595 | <20 |
| 596 | <20 |
| 597 | 26.9 |
| 598 | <20 |
| 599 | <20 |
| 600 | <20 |
| 601 | <20 |
| 602 | <20 |
| 603 | 28.6 |
| 604 | <20 |
| 605 | <20 |
| 606 | 86.7 |
| 607 | <20 |
| 608 | <20 |
| 609 | <20 |
| 610 | <20 |
| 611 | 95.9 |
| 612 | 20.5 |
| 613 | <20 |
| 614 | <20 |
| 615 | 27.4 |
| 616 | <20 |
| 617 | 24.3 |
| 618 | <20 |
| 619 | 39.2 |
| 620 | <20 |
| 621 | 69.4 |
| 622 | 123 |
| 623 | 105 |
| 624 | <20 |

### Test Example 4

### Serotonin 4 (5-HT₄) Receptor Agonist Activity Assay

The cAMP measurement test used herein was carried out by using CISBIO HTRF (trademark) cAMP Hirange kit according to the manufacturer's instructions attached therewith.

CHO cells expressing human 5-HT_{4b} receptor were incubated in Medium 1 [DMEM/1% NEAA, 1% penicillin/streptomycin (P/S), 0.2 mg/mL GENETICIN (G418), 10% FBS] at 37° C. under 5% CO₂ condition. Then, the cells were put into Medium 2 (DMEM/10000 cut FBS, G-418, P/S, NEAA) for 1 to 2 hours, and collected by treating with trypsin containing EDTA. The collected cells were suspended in Assay Buffer 1 [100 mM Hanks/HEPES buffer (pH 7.4)], the suspended cells were mixed with the testing compound on 384-well plates, and the cells were incubated at 31° C. for 15 minutes. To the cells were added cAMP-cryptate solution and cAMP-d₂ solution, and they were incubated at room temperature for 1 hour. Then, time-resolved fluorescence was measured by En Vision (excitation wavelength: 330 nm, fluorescence wavelength: 620/665 nm).

An intrinsic activity of the compound [IA (%)] and a concentration showing 50% of IA [EC₅₀ (nM)] were calculated from the obtained results. In particular, the intrinsic activity (IA) was calculated on the basis of the maximal activity of 5-HT (measured from 10⁻¹¹ M to 10⁻⁷ M) defined as 100%.

Table 100 shows results of serotonin 4 (5-HT₄) receptor agonist activity assay. In the following table, the compounds used in the test are shown in numbers which correspond to the Example numbers above where the preparations of the compounds are described. Each IA and EC₅₀ shows the mean value of each group.

TABLE 100

| Ex. | | EC₅₀ |
|---|---|---|
| No. | IA (%) | (nM) |
| 201 | 39.7 | 6.8 |
| 202 | 39.0 | 5.8 |
| 203 | 71.8 | 1.1 |
| 209 | 79.1 | 3.7 |
| 210 | 68.6 | 5.0 |
| 216 | 62.3 | 4.6 |
| 219 | 96.9 | 2.8 |
| 220 | 92.4 | 4.8 |
| 221 | 86.1 | 6.1 |
| 223 | 72.7 | 3.4 |
| 224 | 73.9 | 6.7 |
| 233 | 93.5 | 2.9 |
| 237 | 51.9 | 9.7 |
| 241 | 58.8 | 3.2 |
| 244 | 63.5 | 3.9 |
| 246 | 56.6 | 21.1 |
| 247 | 79.0 | 12.7 |
| 250 | 79.7 | 3.5 |
| 394 | 101.0 | 3.1 |
| 402 | 106.0 | 1.8 |
| 405 | 21.6 | 34.2 |
| 419 | 110.0 | 10.4 |
| 420 | 85.6 | 8.2 |
| 435 | 1.6 | 11.9 |
| 445 | 52.1 | 13.2 |
| 447 | 56.8 | 12.2 |
| 448 | 57.5 | 8.9 |
| 449 | 76.4 | 31.2 |
| 450 | 87.7 | 15.8 |
| 451 | 83.1 | 13.8 |
| 455 | 99.0 | 28.1 |
| 457 | 72.2 | 12.7 |
| 458 | 85.4 | 2.7 |
| 459 | 68.2 | 7.6 |
| 460 | 78.6 | 5.3 |
| 461 | 65.5 | 4.4 |
| 463 | 64.4 | 8.3 |
| 464 | 70.0 | 31.2 |
| 467 | 72.5 | 6.9 |
| 468 | 69.8 | 22.4 |
| 469 | 68.8 | 5.3 |
| 470 | 70.4 | 13.4 |
| 472 | 54.3 | 47.2 |
| 473 | 58.5 | 23.1 |
| 474 | 89.4 | 34.4 |
| 475 | 61.0 | 13.6 |
| 477 | 65.5 | 11.4 |
| 478 | 68.7 | 32.4 |
| 481 | 73.8 | 7.1 |
| 482 | 70.5 | 8.9 |
| 487 | 70.0 | 3.2 |
| 488 | 106.0 | 2.9 |
| 489 | 104.0 | 2.5 |
| 490 | 111.0 | 1.6 |
| 491 | 91.0 | 4.4 |
| 492 | 78.0 | 2.9 |
| 493 | 75.0 | 1.5 |
| 494 | 72.0 | 2.1 |
| 496 | 70.0 | 2.2 |
| 497 | 88.0 | 5.8 |
| 498 | 36.0 | 3.4 |
| 499 | 82.0 | 7.8 |
| 500 | 90.0 | 14.4 |
| 501 | 84.0 | 3.5 |
| 502 | 70.0 | 3.5 |
| 503 | 98.0 | 5.0 |
| 504 | 47.0 | 3.9 |
| 505 | 52.0 | 5.5 |
| 506 | 46.0 | 2.8 |
| 507 | 84.0 | 3.9 |
| 508 | 54.0 | 3.7 |
| 509 | 56.0 | 16.3 |
| 510 | 46.0 | 2.7 |
| 511 | 51.0 | 9.6 |
| 512 | 30.0 | 4.3 |
| 513 | 81.0 | 10.2 |
| 514 | 70.0 | 12.0 |
| 515 | 70.0 | 8.5 |
| 516 | 75.0 | 7.2 |
| 517 | 72.0 | 6.1 |
| 518 | 59.0 | 5.5 |
| 519 | 81.1 | 33.5 |
| 520 | 82.9 | 17.6 |
| 523 | 24.0 | 2.9 |
| 524 | 84.0 | 4.3 |
| 525 | 86.0 | 18.1 |
| 527 | 114.0 | 5.7 |
| 528 | 121.0 | 17.3 |
| 529 | 95.5 | 1.2 |
| 530 | 97.0 | 3.1 |
| 531 | 95.0 | 11.4 |
| 532 | 99.0 | 5.0 |
| 533 | 102.0 | 3.8 |
| 534 | 104.0 | 5.7 |
| 536 | 100.0 | 3.7 |
| 537 | 111.0 | 1.3 |
| 538 | 76.0 | 2.4 |
| 539 | 75.0 | 2.6 |
| 540 | 106.0 | 15.3 |
| 541 | 79.0 | 9.0 |
| 542 | 90.0 | 3.3 |
| 543 | 92.0 | 3.7 |
| 544 | 58.0 | 4.7 |
| 545 | 60.0 | 5.5 |
| 546 | 63.0 | 20.1 |
| 547 | 54.0 | 14.1 |
| 548 | 73.0 | 6.4 |
| 549 | 91.0 | 5.6 |
| 550 | 85.0 | 5.1 |
| 551 | 70.4 | 18.5 |
| 558 | 109.0 | 1.0 |
| 559 | 81.0 | 3.0 |
| 561 | 109.0 | 8.9 |
| 566 | 90.0 | 3.1 |
| 582 | 91.0 | 3.7 |
| 583 | 84.0 | 5.9 |
| 585 | 109.0 | 2.4 |
| 586 | 105.0 | 2.2 |
| 590 | 101.0 | 2.8 |
| 591 | 97.0 | 2.6 |
| 593 | 99.0 | 2.7 |
| 594 | 106.0 | 2.3 |
| 595 | 93.0 | 6.9 |
| 596 | 82.0 | 4.1 |
| 605 | 113.0 | 2.3 |
| 610 | 103.0 | 9.1 |
| 611 | 94.0 | 42.2 |
| 612 | 82.0 | 14.6 |
| 614 | 93.0 | 10.9 |
| 616 | 105.0 | 13.0 |
| 617 | 104.0 | 8.4 |
| 618 | 32.0 | 6.2 |

### Example 5

### The Effect of Compounds on Scopolamine-Treated Cognitive Impairment

Scopolamine, a muscarinic antagonist, significantly impairs cognition by blocking acetylcholine transmission. Thus, scopolamine-induced cognitive impairment model, one of the AD-like model, has been used to predict pharmacodynamic signals of putative procognitive compounds, utilizing the acetylcholinesterase inhibitor donepezil for illustration (See, Citations 1 and 2). The present inventors investigated the effect of each compound on reversal of scopolamine-induced deficits in performance of Y-maze test in mice, and they also investigated adjunctive effect of compounds with donepezil on reversal of deficits induced by scopolamine and MK801 in mice.

Used animal: ddY mouse (SLC)

### Grouping of Animals

In the experiments, the mice were grouped in the same way using Stat Preclinica (Version 1.03295; Takumi Information Technology Inc.). The selected mice were divided into 5 to 7 groups of 7 to 12 mice using the “completely randomization design by the single variable” program (Analytical program version 1.0.7), with body weight on the testing day. After grouping, p values for Bartlett's test and ANOVA across all groups were greater than 0.2, indicating no significant difference in this parameter among the groups.

### Dosing Method and Schedule

The required amount of each compound was weighed and put into a glass homogenizer. The required amount of 0.5% MC (methyl cellulose) solution was added, and each compound was suspended to give a 10 mg/kg dosing suspension.

The required amount of donepezil was weighed and put into a glass homogenizer. The required amount of 0.5% MC solution was added, and donepezil was suspended to give a suspension at concentration of 1 mg/mL (10 mg/kg dosing suspension).

The required amount of scopolamine and MK801 were weighed, and saline was added to it to give 0.3 mg/mL and 0.015 mg/mL solution, respectively.

90 Min before the test, the mice were orally administered each compound, donepezil, and vehicle (0.5% methyl cellulose, 10 mL/kg). After 60 min, memory impairment was induced by administering scopolamine (0.6 mg/kg, s.c.) with (co-administration) or without (mono-administration) MK801 (0.03 mg/kg, s.c.). The control group received saline (2 mL/kg, s.c.) rather than scopolamine and MK801.

Y-maze test

The Y-maze used herein is a three-arm maze with equal angles between all arms. The mazefloor and walls were constructed from black acrylic resin. The mice were initially placed within one arm, and the sequence and number of arm entries were recorded manually for each mouse over an 8-min period.

Data were processed and analyzed with Microsoft® Office Excel 2003. The alternation behavior was defined as entries into all three arms on consecutive occasions. The percent alternation behavior in each animal was calculated using the following formula, and rounded to one decimal place.

Alternation behavior(%)=[actual alternation/(total arm entries−2)]×100

Restoration ratio of alternation behavior (%) in each animal was calculated using the following formula, and rounded to one decimal place.

y=100×(x−B)/(A−B)

Restoration ratio of alternation behavior in each animal
(%)=y
Alternation behavior in each animal (%)=x
Mean of alternation behavior in vehicle-treated group (group No. 1) (%)=A
Mean of alternation behavior in scopolamine-treated group (group No. 2) (%)=B

Data were expressed as the mean of the percent alternation behavior, the number of total arm entries and the restoration ratio of alternation behavior.

### Definition of Cognitive Impairment Induced by Scopolamine and MK801

Values of Y-maze tests are expressed as mean of alternation behavior (n=7−12).

Alternation behaviors in the scopolamine-treated groups were compared to those in the control groups using Wilcoxon rank sum test (Stat preclinical; Version 1.03295; Takumi Information Technology Inc.) with a two-sided significance level of 0.05. Statistical significance in the scopolamine-treated group compared with the control group (*P<0.05) exhibits cognitive impairment.

### The Effect of Each Compound on Scopolamine Induced Cognitive Impairment

Restoration of alternation behavior was analyzed using Stat Preclinica. Alternation behaviors in the vehicle-treated group were compared to those in the test substance-treated groups using non-parametric Dunnett multiple comparison test (Analytical program version 1.0.2) with a two-sided significance level of 0.05. Statistical significance in the test substance-treated group compared with the scopolamine-treated group (#P<0.05) exhibits reversal of scopolamine-induced deficits in cognition.

The Effect of Co-Administration of Compound 232 and Compound 242 Together with the Acetylcholinesterase Inhibitor Donepezil on Scopolamine Induced Cognitive Impairment

Restoration of alternation behavior was analyzed using Stat Preclinica. Alternation behaviors in the Donepezil (10 mg/kg, p.o.)-treated group were compared to those in the co-administrated groups of test compounds with donepezil (10 mg/kg, p.o.) using non-parametric Dunnett multiple comparison test (Analytical program version 1.0.2) with a two-sided significance level of 0.05. Statistical significance in the co-administered group compared with the donepezil-treated group ($P<0.05) shows that the co-administration increases reversal of cognitive impairment compared with the administration of donepezil alone in scopolamine and MK801-induced deficits.

### CITATIONS

(1) Knox L T, Jing Y, Fleete M S, Collie N D, Zhang H, Liu P. Scopolamine impairs behavioural function and arginine metabolism in the rat dentate gyrus. Neuropharmacology 2011; 61: 1452-62.
(2) Ogura H, Kosasa T, Araki S, Yamanishi Y. Pharmacological properties of donepezil hydrochloride (Aricept®), a drug for Alzheimer's disease. Folia Pharmacol Jpn 2000; 115: 45-51.
(3) Kwon S H, Kim H C, Lee S Y, Jang C G. Loganin improves learning and memory impairments induced by scopolamine in mice. Eur J Pharmacol 2009; 619: 44-9.

The effect of each compound on scopolamine-induced cognitive impairment is shown in Table 101.

The effect of co-administration of compound 232 and 242 with donepezil is shown in Table 102.

TABLE 101
Single-agent administration

| | | | compound | | |
|---|---|---|---|---|---|
| | | | (mg/kg, p.o.) | | |
| | normal | control | 1 | 3 | 10 |
| Example 205 | | | | | |
| (n = 9) | | | | | |
| scopolamine | − | + | + | + | + |
| vehicle | + | + | − | − | − |
| Alternation behavior (%) | 77.0 | 49.1* | 66.9^{#} | 54.8 | 66.7^{#} |
| Example 218 | | | | | |
| (n = 9) | | | | | |
| scopolamine | − | + | + | + | + |
| vehicle | + | + | − | − | − |
| Alternation behavior (%) | 77.0 | 49.1* | 59.9 | 53 | 61.3 |
| Example 137 | | | | | |
| (n = 11 to 12) | | | | | |
| scopolamine | − | + | + | + | + |
| vehicle | + | + | − | − | − |
| Alternation behavior (%) | 69.7 | 49.2* | no | 49.3 | 62.6^{#} |
| | | | data | | |
| Example 232 | | | | | |
| (n = 8 to 10) | | | | | |
| scopolamine | − | + | + | + | + |
| vehicle | + | + | − | − | − |
| Alternation behavior (%) | 74.1 | 48.4* | 66.3^{#} | 63.0^{#} | 53.6 |
| Example 242 | | | | | |
| (n = 7 to 10) | | | | | |
| scopolamine | − | + | + | + | + |
| vehicle | + | + | − | − | − |
| Alternation behavior (%) | 68.9 | 44.4* | 63.4^{#} | 62.9^{#} | 57.5 |

*Statistical significance in the scopolamine-treated group compared with the control group using Wilcoxon rank sum test (with a two-sided significance level of 0.05).
^{#}Statistical significance in the test substance-treated group compared with the scopolamine-treated group using non-parametric Dunnett multiple comparison test (Analytical program version 1.0.2) with a two-sided significance level of 0.05.

TABLE 102
Co-administration with donepezil

| Example 232 | nor- | | Donepezil | compound (mg/kg, p.o.) | | | |
|---|---|---|---|---|---|---|---|
| (n = 11 to 12) | mal | control | 10 mg/kg | 0.03 | 0.1 | 0.03 | 0.1 |
| scopolamine + | − | + | + | + | + | + | + |
| MK801 | | | | | | | |
| vehicle | + | + | − | − | − | − | − |
| Donepezil | − | − | + | − | − | + | + |
| (10 mg/kg, | | | | | | | |
| p.o.) | | | | | | | |
| Alternation | 67.4 | 44.1* | 51.5^{#} | 46.7 | 48.0^{#} | 48.5 | 62.4^{$} |
| behavior (%) | | | | | | | |
| Example 242 | nor- | | Donepezil | compound (mg/kg, p.o.) | | | |
| (n = 8) | mal | control | 10 mg/kg | 0.1 | 0.3 | 0.1 | 0.3 |
| scopolamine + | − | + | + | + | + | + | + |
| MK801 | | | | | | | |
| vehicle | + | + | − | − | − | − | − |
| Donepezil | − | − | + | − | − | + | + |
| (10 mg/kg, | | | | | | | |
| p.o.) | | | | | | | |
| Alternation | 69.7 | 38.2* | 49.9^{#} | 51.2^{#} | 57.4^{#} | 58.6^{$} | 62.5^{$} |
| behavior (%) | | | | | | | |

*Statistical significance in the scopolamine-treated group compared with the control group using Wilcoxon rank sum test with a two-sided significance level of 0.05.
^{#}Statistical significance in the test substance-treated group compared with the scopolamine-treated group using non-parametric Dunnett multiple comparison test with a two-sided significance level of 0.05.
^{$}Statistical significance in the co-treated (donepezil and test compounds) group compared with the donepezil (10 mg/kg)-treated group using non-parametric Dunnett multiple comparison test with a two-sided significance level of 0.05.

### INDICATION

The present inventions are applied to, for example, treating or preventing the diseases or symptoms of the following (i) to (v):

(i) neuropsychiatric diseases such as Alzheimer-type dementia, Lewy body dementia, vascular dementia, depression, posttraumatic stress disorder (PTSD), memory impairment, anxiety, and schizophrenia;
(ii) digestive system diseases such as irritable bowel syndrome, atonic constipation, habitual constipation, chronic constipation, constipation induced by drugs (e.g. morphine and antipsychotic drugs), constipation associated with Parkinson's disease, constipation associated with multiple sclerosis, constipation associated with diabetes mellitus, and constipation or dyschezia caused by contrast materials taken as a pretreatment for endoscopic examinations or barium enema X-ray examinations;
(iii) digestive system diseases such as functional dyspepsia, acute/chronic gastritis, reflux esophagitis, gastric ulcer, duodenal ulcer, gastric neurosis, postoperative paralytic ileus, senile ileus, non-erosive reflux disease, NSAID ulcer, diabetic gastroparesis, postgastrectomy syndrome, and intestinal pseudo-obstruction;
(iv) digestive system symptoms such as the digestive system diseases mentioned in the above (ii) and (iii), scleroderma, diabetes mellitus, anorexia in esophageal/biliary-tract diseases, nausea, emesis, bloating, epigastric discomfort, abdominal pain, heartburn, and belching; and
(v) urinary system diseases associated with dysuria such as urinary tract obstruction and prostatic hyperplasia.

Thus, the present compounds can be used for treating and preventing the various diseases mentioned above (in particular, neuropsychiatric diseases) and abnormal-functions of digestive system associated with the treatment of the various diseases mentioned above and the like. In specific, the present compound is useful as a medicament for treating especially the neuropsychiatric diseases such as Alzheimer-type dementia mentioned in the above (i) because the compound shows an excellent 5-HT4 receptor agonist activity and brain penetration.

In addition, the present compound is expected to show further efficacy in treating the various neuropsychiatric diseases mentioned in the above (i), especially Alzheimer-type dementia, by combining at least one of the following medicaments:

acetylcholinesterase inhibitors such as donepezil, galantamine, rivastigmine, SNX-001 and NP-61; cholinesterase inhibitors such as huperzine A; NMDA receptor antagonists such as memantine, dimebon and neramexane; 5-HT6 receptor antagonists such as PF-5212365 (SAM-531), SB-742457, LU-AE58054, AVN-322, PF-05212377 (SAM-760) and AVN101; α7nAChR agonists such as TC-5619, EVP-6124 and GTS-21; α4β2nACh receptor agonists such as AZD-1446 and CHANTIX (varenicline); nAChR agonists such as ABT-089; AMPA receptor agonists such as CX-717 and LY-451395; histamine H3 antagonists such as ABT-288, SAR-110894 and PF-03654746; muscarinic M1 receptor agonists such as MCD-386 and GSK-1034702; PDE4 inhibitors such as etazolate; PDE9 inhibitors such as PF-04447943; histone deacetylase inhibitors such as EVP-0334; σ1 receptor agonists such as Anavex-2-73; γ-secretase inhibitors (GSI) such as BMS-708163, NIC5-15, ELND-006, and MK-0752; γ-secretase modulators (GSM) such as E-2212 and CHF-5074; Aβ human monoclonal antibodies such as bapineuzumab, solanezumab, PF-4360365 (ponezumab), gantenerumab (R-1450), BAN-2401, MABT-5102A, RG-7412 and GSK-933776A; Aβ vaccines such as ACC-001 (PF-05236806), AD-02, CAD-106, V-950, UB-311 and ACI-24; human immunoglobulins such as GAMMAGARD; Ap aggregation inhibitors such as ELND-005 (AZD-103), PBT-2, NRM-8499 and Exebryl-1; tau aggregation inhibitors such as TRx-0014 and LMTX; RACE inhibitors such as ACI-91, posiphen, CTS-21166, HPP-854 and LY-2886721; tyrosine kinase inhibitors such as masitinib; GSK-3β inhibitors/tau kinase inhibitors such as NP-12; RAGE fusion proteins such as TTP-4000; ApoA-I/HDL-C elevations such as RVX-208; other various agents showing neuroprotective action such as SK-PC-B70M, T-817MA, davunetide, HF-0220, PF-4494700, PYM-50028, CERE-110, ASP-0777, TAK-065, and AAD-2004; and other medicaments used for treating various types of dementia.

### INDUSTRIAL APPLICABILITY

The present compound is useful as a medicament for treating or preventing diseases or symptoms associated with serotonin-4 receptor. The diseases or symptoms suggested to be associated with serotonin-4 receptor include the following (i) to (v):

(i) neuropsychiatric diseases such as Alzheimer-type dementia, Lewy body dementia, vascular dementia, depression, posttraumatic stress disorder (PTSD), memory impairment, anxiety, and schizophrenia;
(ii) digestive system diseases such as irritable bowel syndrome, atonic constipation, habitual constipation, chronic constipation, constipation induced by drugs (e.g. morphine and antipsychotic drugs), constipation associated with Parkinson's disease, constipation associated with multiple sclerosis, constipation associated with diabetes mellitus, and constipation or dyschezia caused by contrast materials taken as a pretreatment for endoscopic examinations or barium enema X-ray examinations;
(iii) digestive system diseases such as functional dyspepsia, acute/chronic gastritis, reflux esophagitis, gastric ulcer, duodenal ulcer, gastric neurosis, postoperative paralytic ileus, senile ileus, non-erosive reflux disease, NSAID ulcer, diabetic gastroparesis, postgastrectomy syndrome, and intestinal pseudo-obstruction;
(iv) digestive system symptoms such as the digestive system diseases mentioned in the above (ii) and (iii), scleroderma, diabetes mellitus, anorexia in esophageal/biliary-tract diseases, nausea, emesis, bloating, epigastric discomfort, abdominal pain, heartburn, and belching; and
(v) urinary system diseases associated with dysuria such as urinary tract obstruction and prostatic hyperplasia.

In addition, the present compound is useful as a medicament for treating or preventing especially the neuropsychiatric diseases such as Alzheimer-type dementia mentioned in the above (i) because the compound shows an excellent 5-HT4 receptor agonist activity and brain penetration.

1. A compound of Formula (1): 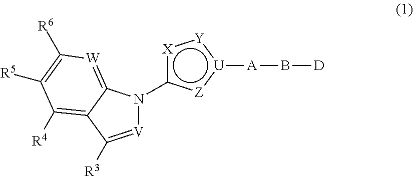
or a pharmaceutically acceptable salt thereof wherein
A is the following Formula (A-1), Formula (A-2), Formula (A-3), or Formula (A-4): 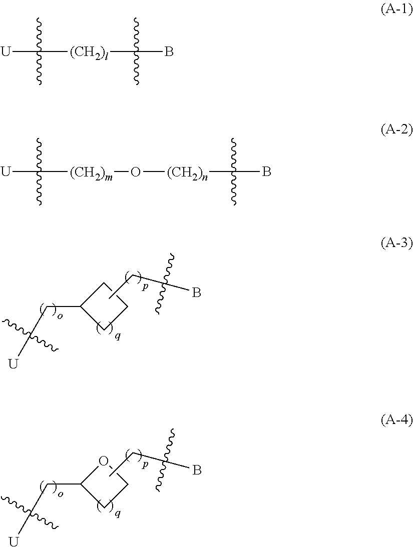
wherein
l is an integer of 0 to 4,
m is an integer of 0 to 2,
n is an integer of 0 to 2,
o and p are independently an integer of 0 or 1,
q is an integer of 0 to 5,
(A-1) to (A-4) may be independently and optionally substituted with one or more substituents independently-selected from the group consisting of C₁₋₆ alkyl group, C₂₋₆ alkenyl group, C₂₋₆ alkynyl group, hydroxy group, C₁₋₆ alkoxy group, and halogen atom at each substitutable position thereof,
B is the following Formula (B-1), Formula (B-2), or Formula (B-3): 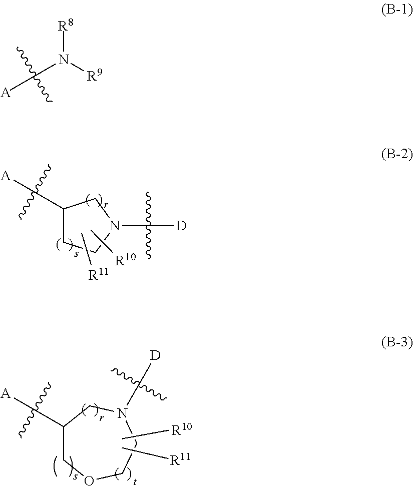
wherein (B-2) and (B-3) may optionally include an unsaturated bond(s) at an acceptable position(s) of the ring, provided that D is absent when B is (B-1),
R⁸, R⁹ and D are independently a group selected from the group consisting of the following (1) and (2):
(1) hydrogen atom, an optionally-substituted C₁₋₆ alkyl group, an optionally-substituted C₃₋₆ alkenyl group, an optionally-substituted C₃₋₆ alkynyl group, an optionally-substituted C₃₋₈ monocyclic, C₇₋₁₀ bicyclic or C₇₋₁₂ tricyclic cycloalkyl group, and an optionally-substituted C₅₋₈ monocyclic or C₇₋₁₀ bicyclic cycloalkenyl group
wherein the C₁₋₆ alkyl group, C₃₋₆ alkenyl group, C₃₋₆ alkynyl group, C₃₋₈ monocyclic, C₇₋₁₀ bicyclic or C₇₋₁₂ tricyclic cycloalkyl group, and C₅₋₈ monocyclic or C₇₋₁₀ bicyclic cycloalkenyl group may be independently and optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, aryl group, heteroaryl group, aryloxy group, C₂₋₆ alkanoyl group, phenacyl group, and halogen atom at each substitutable position thereof;
(2) —(CH₂)ᵤ—R¹²
wherein u is an integer of 0 to 4 provided that when u is an integer of 1 to 4, the alkylene chain may be optionally substituted with one or more substituents independently-selected from the group consisting of C₁₋₆ alkyl group, C₂₋₆ alkenyl group, C₂₋₆ alkynyl group, hydroxy group, C₁₋₆ alkoxy group, oxo group, and halogen atom,
R¹² is the following Formula (R¹²-1), Formula (R¹²-2), Formula (R¹²-3), Formula (R¹²-4), Formula (R¹²-5), Formula (R¹²-6), Formula (R¹²-7), or Formula (R¹²-8): 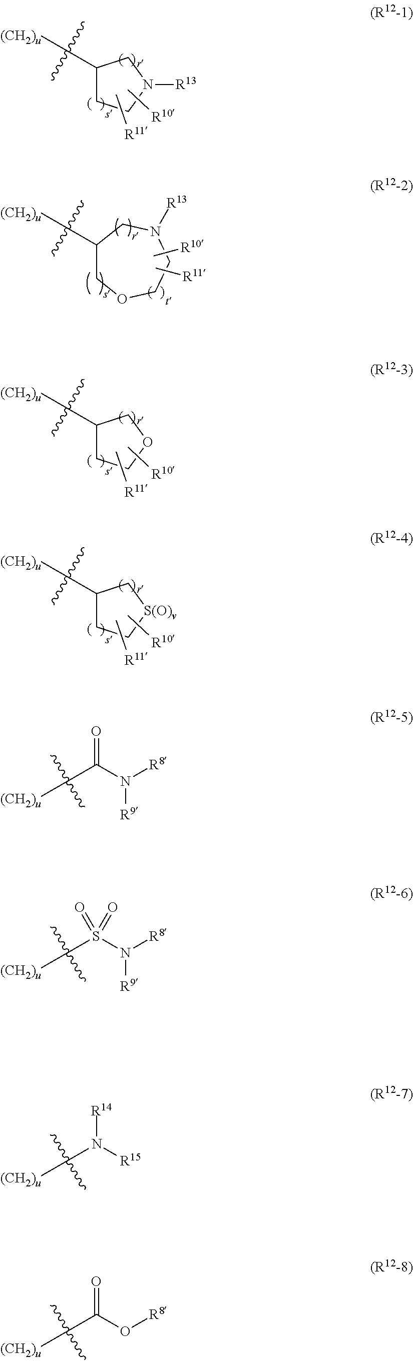
wherein R¹³ is a group selected from the group consisting of the following (1) to (5):
(1) hydrogen atom and formyl group;
(2) an optionally-substituted C₁₋₆ alkyl group, an optionally-substituted C₃₋₆ alkenyl group, an optionally-substituted C₃₋₆ alkynyl group, an optionally-substituted C₃₋₈ cycloalkyl group, and an optionally-substituted C₅₋₈ cycloalkenyl group
wherein the C₁₋₆ alkyl group, C₃₋₆ alkenyl group, C₃₋₆ alkynyl group, C₃₋₈ cycloalkyl group, and C₅₋₈ cycloalkenyl group may be independently and optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, and halogen atom at each substitutable position thereof;
(3) —COR¹⁶, —CSR¹⁶, —SO₂R¹⁶, —CO—COR¹⁶, —COOR¹⁶, and —CO—COOR¹⁶
wherein R¹⁶ is an optionally-substituted C₁₋₆ alkyl group, an optionally-substituted C₃₋₆ alkenyl group, an optionally-substituted C₃₋₆ alkynyl group, an optionally-substituted C₃₋₈ cycloalkyl group, an optionally-substituted C₅₋₈ cycloalkenyl group, an optionally-substituted aryl group, an optionally-substituted heteroaryl group, an optionally-substituted 5- to 9-membered monocyclic or 7- to 10-membered bicyclic non-aromatic unsaturated heterocyclic group (wherein the binding site is any one carbon atom in the heterocyclic ring), or an optionally-substituted 4- to 9-membered monocyclic or 7- to 10-membered bicyclic saturated heterocyclic group (wherein the binding site is any one carbon atom in the heterocyclic ring),
wherein the C₁₋₆ alkyl group, C₃₋₆ alkenyl group, C₃₋₆ alkynyl group, C₃₋₈ cycloalkyl group, C₅₋₈ cycloalkenyl group, 5- to 9-membered monocyclic or 7- to 10-membered bicyclic non-aromatic unsaturated heterocyclic group, and 4- to 9-membered monocyclic or 7- to 10-membered bicyclic saturated heterocyclic group may be independently and optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, aryl group, heteroaryl group, and halogen atom at each substitutable position thereof; and the aryl group and heteroaryl group may be independently and optionally substituted with one or more substituents independently-selected from the group consisting of halogen atom, hydroxy group, C₁₋₄ alkyl group, C₁₋₄ alkoxy group, C₁₋₄ haloalkyl group, C₁₋₄ haloalkoxy group, cyano group, nitro group, C₂₋₆ alkanoyl group, and an optionally-substituted amino group at each substitutable position thereof;
(4) —CONR¹⁷—OR¹⁸
wherein R¹⁷ and R¹⁸ are independently hydrogen atom, C₁₋₆ alkyl group, C₃₋₆ alkenyl group or C₃₋₆ alkynyl group;
(5) —CONR¹⁹R²⁰, —CSNR¹⁹R²⁰ and —SO₂NR¹⁹R²⁰
wherein R¹⁹ and R²⁰ are independently hydrogen atom or any group defined in the said R¹⁶, or
R¹⁹ and R²⁰ may be taken together with the adjacent nitrogen atom to form a saturated or unsaturated 4- to 8-membered monocyclic nitrogen-containing heterocyclic group comprising additional 0 to 2 heteroatoms independently-selected from the group consisting of 1 to 2 nitrogen atoms, 1 oxygen atom and 1 sulfur atom wherein the heterocyclic group may be optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, and halogen atom at each substitutable position thereof,
R¹⁴ and R¹⁵ are independently hydrogen atom, an optionally-substituted C₁₋₆ alkyl group, an optionally-substituted C₃₋₆ alkenyl group, an optionally-substituted C₃₋₆ alkynyl group, an optionally-substituted C₃₋₈ cycloalkyl group, an optionally-substituted C₅₋₈ cycloalkenyl group, an optionally-substituted aryl group, an optionally-substituted heteroaryl group, an optionally-substituted 5- to 9-membered monocyclic or 7- to 10-membered bicyclic non-aromatic unsaturated heterocyclic group (which is attached to the adjacent nitrogen atom via any one carbon atom in the heterocyclic group), an optionally-substituted 4- to 9-membered monocyclic or 7- to 10-membered bicyclic saturated heterocyclic group (which is attached to the adjacent nitrogen atom via any one carbon atom in the heterocyclic group), C₂₋₆ alkanoyl group, C₁₋₆ alkoxycarbonyl group, carbamoyl group, sulfamoyl group, or C₁₋₆ alkylsulfonyl group,
wherein the C₁₋₆ alkyl group, C₃₋₆ alkenyl group, C₃₋₆ alkynyl group, C₃₋₈ cycloalkyl group, C₅₋₈ cycloalkenyl group, 5- to 9-membered monocyclic or 7- to 10-membered bicyclic non-aromatic unsaturated heterocyclic group, 4- to 9-membered monocyclic or 7- to 10-membered bicyclic saturated heterocyclic group, C₂₋₆ alkanoyl group, C₁₋₆ alkoxycarbonyl group, and C₁₋₆ alkylsulfonyl group may be independently and optionally substituted with one or more substituents independently-selected from the group consisting of C₁₋₄ alkyl group, hydroxy group, C₁₋₄ alkoxy group, cyano group, oxo group, aryl group, heteroaryl group, and halogen atom at each substitutable position thereof; and the aryl group and heteroaryl group may be independently and optionally substituted with one or more substituents independently-selected from the group consisting of halogen atom, hydroxy group, C₁₋₄ alkyl group, C₁₋₄ alkoxy group, cyano group, nitro group, C₂₋₆ alkanoyl group, and an optionally-substituted amino group at each substitutable position thereof, or
R¹⁴ and R¹⁵ may be taken together with the adjacent nitrogen atom to form a saturated or unsaturated 4- to 9-membered monocyclic or 7- to 10-membered bicyclic nitrogen-containing heterocyclic group comprising additional 0 to 2 heteroatoms independently-selected from the group consisting of 1 to 2 nitrogen atoms, 1 oxygen atom and 1 sulfur atom wherein the heterocyclic group may be optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, and halogen atom at each substitutable position thereof,
(R¹²-1) to (R¹²-4) may optionally include an unsaturated bond(s) at an acceptable position(s) of the ring,
R⁸′ and R⁹′ are independently hydrogen atom, an optionally-substituted C₁₋₆ alkyl group, an optionally-substituted C₃₋₆ alkenyl group, an optionally-substituted C₃₋₆ alkynyl group, an optionally-substituted C₃₋₈ cycloalkyl group, an optionally-substituted C₅₋₈ cycloalkenyl group, an optionally-substituted aryl group, an optionally-substituted heteroaryl group, an optionally-substituted 5- to 9-membered monocyclic or 7- to 10-membered bicyclic non-aromatic unsaturated heterocyclic group (which is attached to the adjacent nitrogen atom via any one carbon atom in the heterocyclic group), or an optionally-substituted 4- to 9-membered monocyclic or 7- to 10-membered bicyclic saturated heterocyclic group (which is attached to the adjacent nitrogen atom via any one carbon atom in the heterocyclic group),
wherein the C₁₋₆ alkyl group, C₃₋₆ alkenyl group, C₃₋₆ alkynyl group, C₃₋₈ cycloalkyl group, C₅₋₈ cycloalkenyl group, 5- to 9-membered monocyclic or 7- to 10-membered bicyclic non-aromatic unsaturated heterocyclic group, and 4- to 9-membered monocyclic or 7- to 10-membered bicyclic saturated heterocyclic group may be independently and optionally substituted with one or more substituents independently-selected from the group consisting of C₁₋₄ alkyl group, hydroxy group, C₁₋₄ alkoxy group, C₁₋₄ haloalkoxy group, cyano group, oxo group, aryl group, heteroaryl group, aryloxy group, C₂₋₆ alkanoyl group, phenacyl group, and halogen atom at each substitutable position thereof; and the aryl group and heteroaryl group may be independently and optionally substituted with one or more substituents independently-selected from the group consisting of halogen atom, hydroxy group, C₁₋₄ alkyl group, C₁₋₄ alkoxy group, C₁₋₄ haloalkyl group, C₁₋₄ haloalkoxy group, cyano group, nitro group, C₂₋₆ alkanoyl group, and an optionally-substituted amino group at each substitutable position thereof, or a pair of R⁸ and R⁹, and a pair of R⁸ and R⁹′ may be independently taken together with the adjacent nitrogen atom to form a saturated or unsaturated 4- to 9-membered monocyclic or 7- to 10-membered bicyclic nitrogen-containing heterocyclic group comprising additional 0 to 2 heteroatoms independently-selected from the group consisting of 1 to 2 nitrogen atoms, 1 oxygen atom and 1 sulfur atom wherein the nitrogen-containing heterocyclic group may be optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, and halogen atom at each substitutable position thereof,
R¹⁰, R¹⁰′, R¹¹ and R¹¹′ are independently hydrogen atom, halogen atom, hydroxy group, an optionally-substituted C₁₋₆ alkyl group, an optionally-substituted C₂₋₆ alkenyl group, an optionally-substituted C₂₋₆ alkynyl group, an optionally-substituted C₁₋₆ alkoxy group, cyano group, or an oxo group,
wherein the C₁₋₆ alkyl group, C₂₋₆ alkenyl group, C₂₋₆ alkynyl group, and C₁₋₆ alkoxy group may be independently and optionally substituted with one or more substituents independently-selected from the group consisting of C₁₋₄ alkyl group, hydroxy group, C₁₋₄ alkoxy group, C₁₋₄ haloalkoxy group, cyano group, oxo group, aryl group, heteroaryl group, aryloxy group, C₂₋₆ alkanoyl group, phenacyl group, and halogen atom at each substitutable position thereof, or
a pair of R¹⁰ and R¹¹, and a pair of R¹⁰′ and R¹¹′ may be independently taken together to form an optionally-substituted saturated or unsaturated 3- to 8-membered ring that may comprise 1 oxygen atom, which may be a bicyclic or a spiro compound with the ring to which the pair of R¹⁰ and R¹¹, or R¹⁰′ and R¹¹′ is attached,
wherein the saturated or unsaturated 3- to 8-membered ring may be optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, aryl group, heteroaryl group, aryloxy group, C₂₋₆ alkanoyl group, phenacyl group, and halogen atom at each substitutable position thereof,
r and r′ are independently an integer of 0 to 3,
s and s′ are independently an integer of 0 to 3,
t and t′ are independently 1 or 2,
v is an integer of 0 to 2,
provided that not both r and s are 0,
V is nitrogen atom or C—R¹ wherein R¹ is hydrogen atom, halogen atom, an optionally-substituted C₁₋₆ alkyl group, an optionally-substituted C₂₋₆ alkenyl group, an optionally-substituted C₂₋₆ alkynyl group, an optionally-substituted C₃₋₈ cycloalkyl group, an optionally-substituted C₅₋₈ cycloalkenyl group, an optionally-substituted aryl group, or an optionally-substituted heteroaryl group,
wherein the C₁₋₆ alkyl group, C₂₋₆ alkenyl group, C₂₋₆ alkynyl group, C₃₋₈ cycloalkyl group, and C₅₋₈ cycloalkenyl group may be independently and optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, aryl group, heteroaryl group, aryloxy group, C₂₋₆ alkanoyl group, phenacyl group, and halogen atom at each substitutable position thereof; and the aryl group and heteroaryl group may be independently and optionally substituted with one or more substituents independently-selected from the group consisting of halogen atom, hydroxy group, C₁₋₄ alkyl group, C₁₋₄ alkoxy group, C₁₋₄ haloalkyl group, C₁₋₄ haloalkoxy group, cyano group, nitro group, C₂₋₆ alkanoyl group, and an optionally-substituted amino group at each substitutable position thereof,
W is nitrogen atom or C—R² wherein R² is hydrogen atom, halogen atom, hydroxy group, an optionally-substituted C₁₋₆ alkyl group, an optionally-substituted C₂₋₆ alkenyl group, an optionally-substituted C₂₋₆ alkynyl group, an optionally-substituted C₃₋₈ cycloalkyl group, an optionally-substituted C₅₋₈ cycloalkenyl group, an optionally-substituted C₁₋₆ alkoxy group, an optionally-substituted C₁₋₄ haloalkyl group, an optionally-substituted C₁₋₄ haloalkoxy group, cyano group, nitro group, an optionally-substituted aryl group, an optionally-substituted heteroaryl group, or an optionally-substituted amino group,
wherein the C₁₋₆ alkyl group, C₂₋₆ alkenyl group, C₂₋₆ alkynyl group, C₃₋₈ cycloalkyl group, C₅₋₈ cycloalkenyl group, C₁₋₆ alkoxy group, C₁₋₄ haloalkyl group, and C₁₋₄ haloalkoxy group may be independently and optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, aryl group, heteroaryl group, aryloxy group, C₂₋₆ alkanoyl group, phenacyl group, and halogen atom at each substitutable position thereof; and the aryl group and heteroaryl group may be independently and optionally substituted with one or more substituents independently-selected from the group consisting of halogen atom, hydroxy group, C₁₋₄ alkyl group, C₁₋₄ alkoxy group, C₁₋₄ haloalkyl group, C₁₋₄ haloalkoxy group, cyano group, nitro group, C₂₋₆ alkanoyl group, and an optionally-substituted amino group at each substitutable position thereof,
provided that when V is C—R¹, W is nitrogen atom, and when V is nitrogen atom, W is C—R²,
U is carbon atom or nitrogen atom,
X, Y and Z are independently selected from the group consisting of oxygen atom, nitrogen atom, sulfur atom and carbon atom, provided that at least one of X, Y and Z is oxygen atom, sulfur atom, or nitrogen atom,
provided that thiazole and oxazole rings are excluded from the 5-membered heteroaryl ring comprising X, Y and Z,
R³ is hydrogen atom, halogen atom, an optionally-substituted C₁₋₆ alkyl group, an optionally-substituted C₂₋₆ alkenyl group, an optionally-substituted C₂₋₆ alkynyl group, an optionally-substituted C₃₋₈ cycloalkyl group, an optionally-substituted C₅₋₈ cycloalkenyl group, an optionally-substituted C₁₋₆ alkoxy group, an optionally-substituted C₁₋₄ haloalkyl group, an optionally-substituted C₁₋₄ haloalkoxy group, cyano group, nitro group, an optionally-substituted aryl group, an optionally-substituted heteroaryl group, an optionally-substituted 5- to 9-membered monocyclic or 7- to 10-membered bicyclic non-aromatic unsaturated heterocyclic group, or an optionally-substituted 4- to 9-membered monocyclic or 7- to 10-membered bicyclic saturated heterocyclic group,
wherein the C₁₋₆ alkyl group, C₂₋₆ alkenyl group, C₂₋₆ alkynyl group, C₃₋₈ cycloalkyl group, C₅₋₈ cycloalkenyl group, C₁₋₆ alkoxy group, C₁₋₄ haloalkyl group, C₁₋₄ haloalkoxy group, 5- to 9-membered monocyclic or 7- to 10-membered bicyclic non-aromatic unsaturated heterocyclic group, and 4- to 9-membered monocyclic or 7- to 10-membered bicyclic saturated heterocyclic group may be independently and optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, aryl group, heteroaryl group, aryloxy group, C₂₋₆ alkanoyl group, phenacyl group, and halogen atom at each substitutable position thereof; and the aryl group and heteroaryl group may be independently and optionally substituted with one or more substituents independently-selected from the group consisting of halogen atom, hydroxy group, C₁₋₄ alkyl group, C₁₋₄ alkoxy group, C₁₋₄ haloalkyl group, C₁₋₄ haloalkoxy group, cyano group, nitro group, C₂₋₆ alkanoyl group, and an optionally-substituted amino group at each substitutable position thereof,
R⁴ is hydrogen atom, halogen atom, hydroxy group, an optionally-substituted C₁₋₆ alkyl group, an optionally-substituted C₂₋₆ alkenyl group, an optionally-substituted C₂₋₆ alkynyl group, an optionally-substituted C₃₋₈ cycloalkyl group, an optionally-substituted C₅₋₈ cycloalkenyl group, an optionally-substituted C₁₋₆ alkoxy group, an optionally-substituted C₁₋₄ haloalkyl group, an optionally-substituted C₁₋₄ haloalkoxy group, cyano group, nitro group, an optionally-substituted aryl group, an optionally-substituted heteroaryl group, or an optionally-substituted amino group,
wherein the C₁₋₆ alkyl group, C₂₋₆ alkenyl group, C₂₋₆ alkynyl group, C₃₋₈ cycloalkyl group, C₅₋₈ cycloalkenyl group, C₁₋₆ alkoxy group, C₁₋₄ haloalkyl group, and C₁₋₄ haloalkoxy group may be independently and optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, aryl group, heteroaryl group, aryloxy group, C₂₋₆ alkanoyl group, phenacyl group, and halogen atom at each substitutable position thereof; and the aryl group and heteroaryl group may be independently and optionally substituted with one or more substituents independently-selected from the group consisting of halogen atom, hydroxy group, C₁₋₄ alkyl group, C₁₋₄ alkoxy group, C₁₋₄ haloalkyl group, C₁₋₄ haloalkoxy group, cyano group, nitro group, C₂₋₆ alkanoyl group, and an optionally-substituted amino group at each substitutable position thereof, or
R³ and R⁴ may be taken together to form a saturated or unsaturated 6- to 9-membered ring optionally comprising 1 oxygen atom wherein the ring may be optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, and halogen atom at each substitutable position thereof, and
R⁵ and R⁶ are independently hydrogen atom, halogen atom, hydroxy group, an optionally-substituted C₁₋₆ alkyl group, an optionally-substituted C₂₋₆ alkenyl group, an optionally-substituted C₂₋₆ alkynyl group, an optionally-substituted C₃₋₈ cycloalkyl group, an optionally-substituted C₅₋₈ cycloalkenyl group, an optionally-substituted C₁₋₆ alkoxy group, an optionally-substituted C₁₋₄ haloalkyl group, an optionally-substituted C₁₋₄ haloalkoxy group, cyano group, nitro group, an optionally-substituted aryl group, an optionally-substituted heteroaryl group, or an optionally-substituted amino group,
wherein the C₁₋₆ alkyl group, C₂₋₆ alkenyl group, C₂₋₆ alkynyl group, C₃₋₈ cycloalkyl group, C₅₋₈ cycloalkenyl group, C₁₋₆ alkoxy group, C₁₋₄ haloalkyl group, and C₁₋₄ haloalkoxy group may be independently and optionally substituted with one or more substituents independently-selected from the group consisting of C₁₋₄ alkyl group, hydroxy group, C₁₋₄ alkoxy group, C₁₋₄ haloalkyl group, C₁₋₄ haloalkoxy group, cyano group, oxo group, aryl group, heteroaryl group, aryloxy group, C₂₋₆ alkanoyl group, phenacyl group, and halogen atom at each substitutable position thereof; and the aryl group and heteroaryl group may be independently and optionally substituted with one or more substituents independently-selected from the group consisting of halogen atom, hydroxy group, C₁₋₄ alkyl group, C₁₋₄ alkoxy group, C₁₋₄ haloalkyl group, C₁₋₄ haloalkoxy group, cyano group, nitro group, C₂₋₆ alkanoyl group, and an optionally-substituted amino group at each substitutable position thereof.

2. The compound of claim 1 or a pharmaceutically acceptable salt thereof wherein V is nitrogen atom and W is C—R².

3. The compound of claim 1 or a pharmaceutically acceptable salt thereof wherein R³ is hydrogen atom, halogen atom, an optionally-substituted C₁₋₆ alkyl group, an optionally-substituted C₂₋₆ alkenyl group, an optionally-substituted C₂₋₆ alkynyl group, an optionally-substituted C₃₋₈ cycloalkyl group, or an optionally-substituted C₅₋₈ cycloalkenyl group.

4. The compound of claim 1 or a pharmaceutically acceptable salt thereof wherein R⁴ and R⁵ are hydrogen atom, and R² and R⁶ are independently hydrogen atom, halogen atom, an optionally-substituted C₁₋₆ alkyl group, an optionally-substituted C₁₋₆ alkoxy group, an optionally-substituted C₁₋₄ haloalkyl group, an optionally-substituted C₁₋₄ haloalkoxy group, or cyano group.

5. The compound of claim 1 or a pharmaceutically acceptable salt thereof wherein U is carbon atom.

6. The compound of claim 1 or a pharmaceutically acceptable salt thereof wherein X is nitrogen atom, Y is oxygen atom, and Z is nitrogen atom.

7. The compound of claim 1 or a pharmaceutically acceptable salt thereof wherein A is (A-1), and 1 is an integer of 0 or 1.

8. The compound of claim 1 or a pharmaceutically acceptable salt thereof wherein B is (B-2), s is an integer of 1, and r is an integer of 1 or 2.

9. The compound of claim 1 which has a chemical structure of Formula (12): 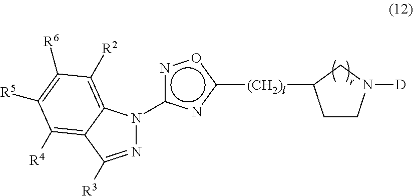
or a pharmaceutically acceptable salt thereof.

10. The compound of claim 1 or a pharmaceutically acceptable salt thereof wherein D is hydrogen atom, an optionally-substituted C₁₋₆ alkyl group, or an optionally-substituted C₃₋₈ monocyclic, C₇₋₁₀ bicyclic or C₇₋₁₂ tricyclic cycloalkyl group.

11. The compound of claim 1 or a pharmaceutically acceptable salt thereof wherein D is —(CH₂)ᵤ—R¹², and R¹² is Formula (R¹²-3).

12. The compound of claim 1 or a pharmaceutically acceptable salt thereof wherein D is —(CH₂)ᵤ—R¹², and R¹² is Formula (R¹²-1).

13. The compound of claim 1 or a pharmaceutically acceptable salt thereof wherein A is (A-3), o is an integer of 0, p is an integer of 0, q is an integer of either 1 or 3, and B is (B-1).

14. The compound of claim 1 which has a chemical structure of Formula (13): 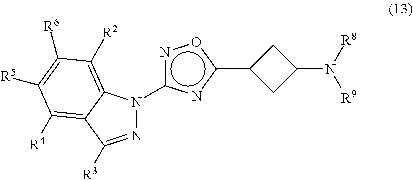
or a pharmaceutically acceptable salt thereof.

15. The compound of claim 1 which is selected from the group consisting of the following compounds or a pharmaceutically acceptable salt thereof:
(01) 1-{5-[1-(3-methoxypropyl)piperidin-4-yl]-1,2,4-oxadiazol-3-yl}-3-(propan-2-yl)-1H-indazole,
(02) 3-ethyl-1-{5-[1-(3-methoxypropyl)piperidin-4-yl]-1,2,4-oxadiazol-3-yl}-1H-indazole,
(03) 3-cyclopropyl-1-{5-[1-(3-methoxypropyl)piperidin-4-yl]-1,2,4-oxadiazol-3-yl}-1H-indazole,
(04) 3-ethyl-6-fluoro-1-{5-[1-(3-methoxypropyl)piperidin-4-yl]-1,2,4-oxadiazol-3-yl}-1H-indazole,
(05) 3-ethyl-7-fluoro-1-{5-[1-(3-methoxypropyl)piperidin-4-yl]-1,2,4-oxadiazol-3-yl}-1H-indazole,
(06) 1-{5-[1-(2-methylpropyl)piperidin-4-yl]-1,2,4-oxadiazol-3-yl}-3-(propan-2-yl)-1H-indazole,
(07) 1-{5-[1-(butan-2-yl)piperidin-4-yl]-1,2,4-oxadiazol-3-yl}-3-ethyl-1H-indazole,
(08) 1-{5-[1-(butan-2-yl)piperidin-4-yl]-1,2,4-oxadiazol-3-yl}-3-cyclopropyl-1H-indazole,
(09) 3-ethyl-1-{5-[1-(2-methylpropyl)piperidin-4-yl]-1,2,4-oxadiazol-3-yl}-1H-indazole,
(10) 1-{5-[1-(cyclopropylmethyl)piperidin-4-yl]-1,2,4-oxadiazol-3-yl}-3-ethyl-1H-indazole,
(11) 1-{5-[1-(butan-2-yl)piperidin-4-yl]-1,2,4-oxadiazol-3-yl}-3-cyclobutyl-1H-indazole,
(12) 3-cyclobutyl-1-{5-[1-(2-methylpropyl)piperidin-4-yl]-1,2,4-oxadiazol-3-yl}-1H-indazole,
(13) 3-(propan-2-yl)-1-[5-(1-propylpiperidin-4-yl)-1,2,4-oxadiazol-3-yl]-1H-indazole,
(14) 3-ethyl-6-fluoro-1-(5-{1-[2-(tetrahydrofuran-2-yl)ethyl]piperidin-4-yl}-1,2,4-oxadiazol-3-yl)-1H-indazole,
(15) 3-ethyl-1-{5-[1-(tetrahydrofuran-2-ylmethyl)piperidin-4-yl]-1,2,4-oxadiazol-3-yl}-1H-indazole,
(16) 3-ethyl-6-fluoro-1-{5-[1-(tetrahydro-2H-pyran-4-ylmethyl)piperidin-4-yl]-1,2,4-oxadiazol-3-yl}-1H-indazole,
(17) 3-ethyl-6-fluoro-1-(5-{1-[2-(tetrahydro-2H-pyran-4-yl)ethyl]piperidin-4-yl}-1,2,4-oxadiazol-3-yl)-1H-indazole,
(18) 3-ethyl-6-fluoro-1-{5-[1-(tetrahydrofuran-3-yl)piperidin-4-yl]-1,2,4-oxadiazol-3-yl}-1H-indazole,
(19) 3-ethyl-6-fluoro-1-{5-[1-(propan-2-yl)piperidin-4-yl]-1,2,4-oxadiazol-3-yl}-1H-indazole,
(20) methyl 4-({4-[3-(3-ethyl-6-fluoro-1H-indazol-1-yl)-1,2,4-oxadiazol-5-yl]piperidin-1-yl}methyl)piperidine-1-carboxylate,
(21) methyl (2S)-2-({4-[3-(3-ethyl-1H-indazol-1-yl)-1,2,4-oxadiazol-5-yl]piperidin-1-yl}methyl)pyrrolidine-1-carboxylate,
(22) 2-fluoroethyl (2S)-2-({4-[3-(3-ethyl-7-fluoro-1H-indazol-1-yl)-1,2,4-oxadiazol-5-yl]piperidin-1-yl}methyl)pyrrolidine-1-carboxylate,
(23) 2-fluoroethyl (3S)-3-({4-[3-(3-ethyl-1H-indazol-1-yl)-1,2,4-oxadiazol-5-yl]piperidin-1-yl}methyl)pyrrolidine-1-carboxylate,
(24) 1-[3-({4-[3-(3-ethyl-7-fluoro-1H-indazol-1-yl)-1,2,4-oxadiazol-5-yl]piperidin-1-yl}methyl)azetidin-1-yl]-2-methoxyethanone,
(25) 1-{4-[3-(3-ethyl-6-fluoro-1H-indazol-1-yl)-1,2,4-oxadiazol-5-yl]-1,4′-bipiperidin-1′-yl}ethanone,
(26) 1-{4-[3-(3-ethyl-7-fluoro-1H-indazol-1-yl)-1,2,4-oxadiazol-5-yl]-1,4′-bipiperidin-1′-yl}ethanone,
(27) methyl 4-[3-(3-ethyl-6-fluoro-1H-indazol-1-yl)-1,2,4-oxadiazol-5-yl]-1,4′-bipiperidine-1′-carboxylate,
(28) 1-(4-{3-[7-fluoro-3-(propan-2-yl)-1H-indazol-1-yl]-1,2,4-oxadiazol-5-yl}-1,4′-bipiperidin-1′-yl)ethanone,
(29) 1-(4-{3-[7-fluoro-3-(propan-2-yl)-1H-indazol-1-yl]-1,2,4-oxadiazol-5-yl}-1,4′-bipiperidin-1′-yl)-2-hydroxyethanone,
(30) methyl 4-{3-[3-(3-ethyl-1H-indazol-1-yl)-1,2,4-oxadiazol-5-yl]azetidin-1-yl}piperidine-1-carboxylate,
(31) 3-{4-[3-(3-ethyl-1H-indazol-1-yl)-1,2,4-oxadiazol-5-yl]piperidin-1-yl}propan-1-ol,
(32) cis-N-ethyl-3-[3-(3-ethyl-6-fluoro-1H-indazol-1-yl)-1,2,4-oxadiazol-5-yl]cyclobutanamine,
(33) 1-[(3R)-3-({4-[3-(3-ethyl-7-fluoro-1H-indazol-1-yl)-1,2,4-oxadiazol-5-yl]piperidin-1-yl}methyl)pyrrolidin-1-yl]ethanone,
(34) 1-[(3R)-3-({4-[3-(3-ethyl-7-fluoro-1H-indazol-1-yl)-1,2,4-oxadiazol-5-yl]piperidin-1-yl}methyl)pyrrolidin-1-yl]-2-methoxyethanone,
(35) 1-[(3R)-3-({4-[3-(3-ethyl-7-fluoro-1H-indazol-1-yl)-1,2,4-oxadiazol-5-yl]piperidin-1-yl}methyl)pyrrolidin-1-yl]-2-hydroxyethanone,
(36) 1-{4-[3-(3-ethyl-7-fluoro-1H-indazol-1-yl)-1,2,4-oxadiazol-5-yl]-1,4′-bipiperidin-1′-yl}-2-hydroxyethanone,
(37) 1-{4-[3-(3-ethyl-7-fluoro-1H-indazol-1-yl)-1,2,4-oxadiazol-5-yl]-1,4′-bipiperidin-1′-yl}-2-methoxyethanone,
(38) 4-[3-(3-ethyl-7-fluoro-1H-indazol-1-yl)-1,2,4-oxadiazol-5-yl]-1′-(methylsulfonyl)-1,4′-bipiperidine,
(39) 1-(4-{3-[7-fluoro-3-(propan-2-yl)-1H-indazol-1-yl]-1,2,4-oxadiazol-5-yl}-1,4′-bipiperidin-1′-yl)-2-methoxyethanone,
(40) 1-[(3S)-3-({4-[3-(3-ethyl-7-fluoro-1H-indazol-1-yl)-1,2,4-oxadiazol-5-yl]piperidin-1-yl}methyl)pyrrolidin-1-yl]ethanone,
(41) 1-[(3S)-3-({4-[3-(3-ethyl-7-fluoro-1H-indazol-1-yl)-1,2,4-oxadiazol-5-yl]piperidin-1-yl}methyl)pyrrolidin-1-yl]-2-methoxyethanone,
(42) 3-ethyl-7-fluoro-1-[5-(1-{[(3S)-1-(methylsulfonyl)pyrrolidin-3-yl]methyl}piperidin-4-yl)-1,2,4-oxadiazol-3-yl]-1H-indazole,
(43) 3-ethyl-7-fluoro-1-[5-(1-{[(3R)-1-(methylsulfonyl)pyrrolidin-3-yl]methyl}piperidin-4-yl)-1,2,4-oxadiazol-3-yl]-1H-indazole,
(44) 1-[4-({4-[3-(3-ethyl-7-fluoro-1H-indazol-1-yl)-1,2,4-oxadiazol-5-yl]piperidin-1-yl}methyl)piperidin-1-yl]-2-hydroxyethanone,
(45) 1-[3-({4-[3-(3-ethyl-7-fluoro-1H-indazol-1-yl)-1,2,4-oxadiazol-5-yl]piperidin-1-yl}methyl)azetidin-1-yl]-2-hydroxyethanone,
(46) 1-{3-[(4-{3-[7-fluoro-3-(propan-2-yl)-1H-indazol-1-yl]-1,2,4-oxadiazol-5-yl}piperidin-1-yl)methyl]azetidin-1-yl}-2-methoxyethanone,
(47) 1-{3-[(4-{3-[7-fluoro-3-(propan-2-yl)-1H-indazol-1-yl]-1,2,4-oxadiazol-5-yl}piperidin-1-yl)methyl]azetidin-1-yl}ethanone,
(48) methyl 3-[(4-{3-[7-fluoro-3-(propan-2-yl)-1H-indazol-1-yl]-1,2,4-oxadiazol-5-yl}piperidin-1-yl)methyl]azetidine-1-carboxylate,
(49) 1-[3-({4-[3-(3-ethyl-7-fluoro-1H-indazol-1-yl)-1,2,4-oxadiazol-5-yl]piperidin-1-yl}methyl)azetidin-1-yl]ethanone,
(50) 1-{(2R)-2-[(4-{3-[7-fluoro-3-(propan-2-yl)-1H-indazol-1-yl]-1,2,4-oxadiazol-5-yl}piperidin-1-yl)methyl]pyrrolidin-1-yl}-2-hydroxyethanone,
(51) 1-(4-{3-[7-fluoro-3-(propan-2-yl)-1H-indazol-1-yl]-1,2,4-oxadiazol-5-yl}-3′-methyl-1,4′-bipiperidin-1′-yl)-2-hydroxyethanone,
(52) 1-(3-{[(3R)-3-({3-[7-fluoro-3-(propan-2-yl)-1H-indazol-1-yl]-1,2,4-oxadiazol-5-yl}methyl)pyrrolidin-1-yl]methyl}azetidin-1-yl)ethanone,
(53) 1-(3-{[(3R)-3-({3-[7-fluoro-3-(propan-2-yl)-1H-indazol-1-yl]-1,2,4-oxadiazol-5-yl}methyl)pyrrolidin-1-yl]methyl}azetidin-1-yl)-2-hydroxyethanone,
(54) 1-[(3S)-3-{[(3R)-3-({3-[7-fluoro-3-(propan-2-yl)-1H-indazol-1-yl]-1,2,4-oxadiazol-5-yl}methyl)pyrrolidin-1-yl]methyl}pyrrolidin-1-yl]ethanone,
(55) 1-[(3S)-3-{[(3R)-3-({3-[7-fluoro-3-(propan-2-yl)-1H-indazol-1-yl]-1,2,4-oxadiazol-5-yl}methyl)pyrrolidin-1-yl]methyl}pyrrolidin-1-yl]-2-hydroxyethanone,
(56) 1-[(3R)-3-{[(3R)-3-({3-[7-fluoro-3-(propan-2-yl)-1H-indazol-1-yl]-1,2,4-oxadiazol-5-yl}methyl)pyrrolidin-1-yl]methyl}pyrrolidin-1-yl]-2-hydroxyethanone,
(57) 1-[(2S)-2-{[(3S)-3-({3-[7-fluoro-3-(propan-2-yl)-1H-indazol-1-yl]-1,2,4-oxadiazol-5-yl}methyl)pyrrolidin-1-yl]methyl}pyrrolidin-1-yl]-2-hydroxyethanone,
(58) 1-[(2R)-2-{[(3S)-3-({3-[7-fluoro-3-(propan-2-yl)-1H-indazol-1-yl]-1,2,4-oxadiazol-5-yl}methyl)pyrrolidin-1-yl]methyl}pyrrolidin-1-yl]-2-hydroxyethanone,
(59) 1-[(3S)-3-{[(3S)-3-({3-[7-fluoro-3-(propan-2-yl)-1H-indazol-1-yl]-1,2,4-oxadiazol-5-yl}methyl)pyrrolidin-1-yl]methyl}pyrrolidin-1-yl]-2-hydroxyethanone,
(60) 1-[(3R)-3-{[(3S)-3-({3-[7-fluoro-3-(propan-2-yl)-1H-indazol-1-yl]-1,2,4-oxadiazol-5-yl}methyl)pyrrolidin-1-yl]methyl}pyrrolidin-1-yl]-2-hydroxyethanone,
(61) 1-{4-[3-(3-ethyl-7-fluoro-1H-indazol-1-yl)-1,2,4-oxadiazol-5-yl]-4′-methyl-1,4′-bipiperidin-1′-yl}-2-hydroxyethanon,
(62) 1-{4-[3-(3-ethyl-7-fluoro-1H-indazol-1-yl)-1,2,4-oxadiazol-5-yl]-4′-methyl-1,4′-bipiperidin-1′-yl}-2-methoxyethanone,
(63) (2S)-1-{4-[3-(3-ethyl-7-fluoro-1H-indazol-1-yl)-1,2,4-oxadiazol-5-yl]-4′-methyl-1,4′-bipiperidin-1′-yl}-2-hydroxypropan-1-one,
(64) 1-[(3S)-3-({4-[3-(3-ethyl-7-fluoro-1H-indazol-1-yl)-1,2,4-oxadiazol-5-yl]piperidin-1-yl}methyl)pyrrolidin-1-yl]-2-hydroxyethanone,
(65) 1-[(2S)-2-({4-[3-(3-ethyl-7-fluoro-1H-indazol-1-yl)-1,2,4-oxadiazol-5-yl]piperidin-1-yl}methyl)pyrrolidin-1-yl]-2-hydroxyethanone,
(66) 1-{4-[(3S)-3-{[3-(3-ethyl-7-fluoro-1H-indazol-1-yl)-1,2,4-oxadiazol-5-yl]methyl}pyrrolidin-1-yl]piperidin-1-yl}ethanone,
(67) 1-{4-[(3R)-3-({3-[7-fluoro-3-(propan-2-yl)-1H-indazol-1-yl]-1,2,4-oxadiazol-5-yl}methyl)pyrrolidin-1-yl]piperidin-1-yl}-2-methoxyethanone,
(68) 1-(3-{[(3R)-3-({3-[7-fluoro-3-(propan-2-yl)-1H-indazol-1-yl]-1,2,4-oxadiazol-5-yl}methyl)pyrrolidin-1-yl]methyl}azetidin-1-yl)-2-methoxyethanone,
(69) 1-[(3S)-3-{[(3R)-3-({3-[7-fluoro-3-(propan-2-yl)-1H-indazol-1-yl]-1,2,4-oxadiazol-5-yl}methyl)pyrrolidin-1-yl]methyl}pyrrolidin-1-yl]-2-methoxyethanone,
(70) 1-[(3R)-3-{[(3R)-3-({3-[7-fluoro-3-(propan-2-yl)-1H-indazol-1-yl]-1,2,4-oxadiazol-5-yl}methyl)pyrrolidin-1-yl]methyl}pyrrolidin-1-yl]-2-methoxyethanone,
(71) 1-{4-[(3S)-3-({3-[7-fluoro-3-(propan-2-yl)-1H-indazol-1-yl]-1,2,4-oxadiazol-5-yl}methyl)pyrrolidin-1-yl]piperidin-1-yl}-2-methoxyethanone,
(72) 1-(3-{[(3S)-3-({3-[7-fluoro-3-(propan-2-yl)-1H-indazol-1-yl]-1,2,4-oxadiazol-5-yl}methyl)pyrrolidin-1-yl]methyl}azetidin-1-yl)-2-methoxyethanone,
(73) 1-[(3S)-3-{[(3S)-3-({3-[7-fluoro-3-(propan-2-yl)-1H-indazol-1-yl]-1,2,4-oxadiazol-5-yl}methyl)pyrrolidin-1-yl]methyl}pyrrolidin-1-yl]-2-methoxyethanone, and
(74) 1-(4-{3-[7-fluoro-3-(propan-2-yl)-1H-indazol-1-yl]-1,2,4-oxadiazol-5-yl}-3′-methyl-1,4′-bipiperidin-1′-yl)ethanone.

16. A pharmaceutical composition comprising the compound of claim 1 or a pharmaceutically acceptable salt thereof.

17. A serotonin-4 receptor agonist comprising the compound of claim 1 or a pharmaceutically acceptable salt thereof as an active ingredient.

18. A medicament for treating Alzheimer-type dementia comprising the compound of claim 1 or a pharmaceutically acceptable salt thereof as an active ingredient.

19. A method for treating a disease associated with serotonin-4 receptor comprising administering a therapeutically effective amount of the compound of claim 1 or a pharmaceutically acceptable salt thereof to a patient in need thereof.

20. A method for treating Alzheimer-type dementia comprising administering a therapeutically effective amount of the compound of claim 1 or a pharmaceutically acceptable salt thereof to a patient in need thereof.
